(19) United States
(12) Patent Application Publication
(10) Pub. No.: US 2009/0215778 A1
(43) Pub. Date: Aug. 27, 2009

(54) Alpha-substituted Arylmethyl Piperazine Pyrazolo [1,5-alpha]Pyrimidine Amide Derivatives

(75) Inventors: Theodore J. Nitz, Boyds, MD (US); Karl Salzwedel, Olney, MD (US); Catherine Finnegan, Baltimore, MD (US); Shirley Brunton, Windsor (GB); Stuart Flanagan, Wallingford (GB); Christian Montalbetti, Wallingford (GB); Thomas Stephen Coulter, Oxfordshire Wantage (GB)

Correspondence Address: STERNE, KESSLER, GOLDSTEIN & FOX P.L.L.C., 1100 NEW YORK AVENUE, N.W., WASHINGTON, DC, 20005, US

(73) Assignee: Panacos Pharmaceuticals, Inc., Gaithersburg, MD (US)

(21) Appl. No.: 12/149,178

(22) Filed: Apr. 28, 2008

Related U.S. Application Data
(60) Provisional application No. 60/914,509, filed on Apr. 27, 2007.

(51) Int. Cl. C07D 487/04; A61K 31/496; A61K 31/519; A61K 31/5377; A61P 31/18

(52) U.S. Cl. 514/235.8; 544/281; 514/252.16; 514/252.11; 544/121

(57) ABSTRACT

Derivatives of pyrazolopyrimidine compounds represented by Formula I are disclosed:

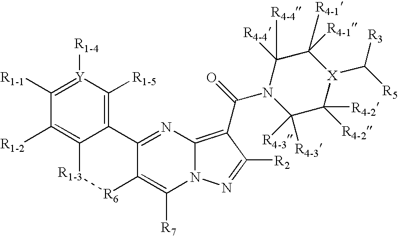

These pyrazolopyrimidine derivatives and pharmaceutical compositions comprising these derivatives are useful in the treatment of HIV mediated diseases and conditions.

This application claims the benefit of U.S. provisional application No. 60/914,509, filed Apr. 27, 2007, the disclosure of which is incorporated by reference herein in its entirety.

### FIELD OF THE INVENTION

The present invention concerns novel pharmaceutically active pyrazolo[1,5-a]pyrimidine derivatives, pharmaceutical compositions containing the same, their use as medicaments, and the use of the compounds for the manufacture of specific medicaments. The present invention also concerns a method of treatment involving administration of the compounds.

The novel compounds are useful as antiretroviral agents. In particular, the novel compounds are useful for the treatment of Human Immunodeficiency Virus (HIV-1).

### BACKGROUND OF THE INVENTION

Human Immunodeficiency Virus (HIV) is a retrovirus that infects and invades cells of the immune system; it breaks down the body's immune system and renders the patient susceptible to opportunistic infections and neoplasms. The immune defect appears to be progressive and irreversible, with a high mortality rate that approaches 100% over several years.

The inherent tendency of HIV to mutate and become resistant to antiretroviral drugs remains a challenge to successful treatment. Patients with drug-resistant strains have an increasing risk of treatment failure with each subsequent treatment regimen. The concurrent use of multiple antiretroviral drugs has been reported to provide an improved virologic response and reduced probability for viral mutations.

Drug-resistant strains of HIV often appear over extended periods of time, even when the patient is on combination therapy. In some cases where an HIV strain is resistant to one drug in a therapeutic class, the strain will sometimes be cross-resistant to similar drugs in the same therapeutic class. Cross-resistance is a particular problem if a patient develops cross-resistance to a whole group of drugs. For example, if a patient has a cross-resistant strain exhibiting resistance to one non-nucleoside reverse transcriptase inhibitor (“NNRTI”), there is a risk that the patient's strain will be resistant to the entire NNRTI class.

Drugs acting by novel mechanisms of action are needed to treat patients harboring viruses that have mutated to develop resistance to currently approved drugs. Early stage events including viral attachment, fusion and entry have emerged as viable targets of potentially significant therapeutic utility.

The HIV-1 envelope is a 160 kDa glycoprotein that is cleaved to form a transmembrane subunit and a surface subunit. The transmembrane subunit is termed “gp41.” The surface subunit is termed “gp120.” Crystallographic analyses of portions of both gp120 and gp41 have contributed to the knowledge base surrounding the biochemistry of HIV-1 envelope. (Kwong, P. D., et al., Nature (London) 393:648-659 (1998); Chan, D. C., et al., Cell 89:263-273 (1997); Weissenhorn, W., et al., Nature 387:426-430 (1997)). It is believed that gp120 and gp41 are held together by van der Waals forces and hydrogen bonding. The gp120/gp41 complex is present as a trimer on the virion surface where it mediates viral attachment, fusion and entry.

HIV-1 infection is initiated by the attachment of gp120 to the CD4 receptor on the cell surface (Salzwedel, K., et al., J. Virol. 74:326-333 (2000)). Studies indicate that upon binding to CD4, a conformational change occurs in gp120, resulting in the repositioning of the V1 and V2 loops of gp120, and exposure of the gp120 bridging sheet domain. The bridging sheet, along with the V3 loop of gp120, is then available for binding to a co-receptor on the cell surface, predominantly either CXCR4 or CCR5, (Kwong et al., 1998, Rizzuto et al., 1998, Zhang et al., 1999). In laboratory-adapted HIV-1 isolates utilizing the CXCR4 co-receptor, CD4 binding also appears to induce conformational changes in gp41 that result in the formation of an extended intermediate structure (Furuta, R. A., et al., Nat. Struct. Biol. 5:276-279 (1998); de Rosny, E., et al., J. Virol. 75:8859-8863 (2001); Kilgore, N. R., et al., J. Virol. 77:7669-7672 (2003)). In this extended intermediate conformation, the N-terminal fusion peptide is believed to project outwards toward the cellular membrane. This conformation appears to be stabilized by the formation of a trimeric coiled-coil structure by the N-terminal heptad repeat regions of the three gp41 subunits within the Env trimer (Chen, D. C. and Kim, P. S., Cell 93:681-684 (1998)). Whether these gp41 conformational changes occur before or after co-receptor binding in primary, CCR5-using HIV-1 isolates is unclear. However, in both cases, binding of the CD4-bound gp120 subunit to co-receptor allows the N-terminal fusion peptide of gp41 to insert into and disrupt the cellular membrane (Salzwedel et al., 2000; Finnegan, C. M., et al., J. Virol. 76:12123-12134 (2002)). The gp120 subunit then undergoes further conformational changes, perhaps induced by interaction with co-receptor, which may result in the dissociation of gp120 from gp41. These gp120 conformational changes, in turn, induce a final conformational change in gp41 in which the protein refolds into a hairpin configuration. This hairpin conformation is stabilized by the binding of the C-terminal heptad repeat regions of gp41 into the outer grooves on the trimeric N-terminal heptad repeat coiled-coil intermediate structure. These conformational changes culminate in the formation of a six-helix bundle structure, which promotes fusion of the viral and cellular membranes by bringing them into closer proximity. Formation of the six-helix bundle is required in order for Env-mediated fusion to occur (Eckert, D. M. and Kim, P. S., Annu. Rev. Biochem. 70:777-810 (2001); Weiss, C. D., AIDS Rev. 5:214-221 (2003)). After a fusion pore is formed, the viral core is released into the cellular cytoplasm, thus initiating infection.

HIV-1 entry inhibitors can generally be divided into three classifications: 1) attachment inhibitors, which inhibit virion attachment to the cell; 2) co-receptor antagonists, which interact with co-receptor to block its binding to gp120, and 3) fusion inhibitors, which interact with Env and disrupt conformational changes that are required for fusion of the viral and cell membranes.

Fusion inhibitors are a relatively new class of antiretrovirals. FUZEON (enfuvirtide) is the first FDA approved drug that acts at a target other than reverse transcriptase or protease. Enfuvirtide inhibits Env-mediated fusion by preventing formation of the gp41 six-helix bundle structure (Matthews, T., et al., Nat. Rev. Drug Discov. 3:215-225 (2004); Kilgore et al., 2003). While the introduction of a new class of antiretrovirals represents an advance in the medicinal arts, enfuvirtide must be injected twice daily with a specialized device. Also, some clinicians have reported non-compliance with treatment regimens due to injection site reactions associated with enfuvirtide treatment. Despite its drawbacks, enfuvirtide has provided proof-of-concept for the validity of viral fusion/entry as a therapeutic target.

As used herein, “fusion inhibitor” refers to inhibition of at least one of the following steps:
1. Gp120 binding to the CD4 receptor of the target cell;
2. Changes in the conformation of gp120 that are induced by binding to the CD4 receptor;
3. Exposure of the co-receptor binding site on gp120;
4. Changes in the conformation of gp41 that are induced by binding of gp120 to the CD4 receptor;
5. Exposure of the N-terminal fusion peptide in gp41;
6. Formation of the N-heptad repeat coiled-coil structure and extended intermediate conformation of gp41;
7. Gp120 binding to a target cell co-receptor protein, for example, the chemokine receptors CCR5 and CXCR4;
8. Changes in the conformation of gp120 that are induced by binding to co-receptor;
9. Insertion of the gp41 amino terminus into the target cell membrane;
10. Release of gp120 from the gp41-gp120 complex;
11. Changes in the conformation of gp41 that are induced by binding of gp120 to co-receptor;
12. Formation of the six-helix bundle (“6HB”) structure in gp41;
13. Fusion of the membranes of the virion particle and target cell;
14. Entry of viral cores into the cellular cytoplasm; and,
15. Non-specific attachment to the target cell via cell surface sugars such as syndecans, C-type lectin receptors (“DC-SIGN” or “CD209”), and heparan sulfate proteoglycans (“HSPG”).

Published PCT patent application no. WO2004/089471 refers to pyrazolo[1,5-a]pyrimidine derivatives and the use thereof as anti-type 2 diabetic agents. WO2004/089471 does not disclose any compounds comprising an alkylaryl group at the 4 position of the piperazine ring.

U.S. Pat. No. 5,602,137 refers to pyrimidine derivatives as angiotensin-II inhibitors (“AII-i”) that are structurally distinct from the compounds of the present invention.

Published U.S. patent application no. 2005/0090522 refers to azaindoleoxoacetic acid derivatives as anti-HIV therapeutics. All compounds mentioned in the 2005/0090522 application comprise: 1) a pyrrolopyridinyl core; and 2) a carbonyl group bonded to both the 1 and 4 position nitrogens of the piperazine ring.

Despite recent progress in the development of HIV therapeutic options, there remains a need for drugs having different or enhanced anti-HIV properties relative to currently marketed pharmaceuticals.

One technical problem underlying the invention relates to the specific need for a small chemical entity fusion inhibitor.

Another technical problem underlying the invention relates to the specific need for an orally bioavailable fusion inhibitor.

Another technical problem underlying the invention relates to the specific need for a compound that inhibits viral entry by mediating the interaction of HIV Env with a cell surface receptor, for example CD-4.

Another technical problem underlying the invention relates to the specific need for a compound that inhibits viral entry by mediating the interaction of HIV Env with a co-receptor, for example either or both of the CXCR4 and CCR5 co-receptors.

### BRIEF SUMMARY OF THE INVENTION

It has been discovered that compounds of Formula I below are unique compositions exhibiting antiretroviral properties.

One aspect of the present invention is directed to compounds of Formula I.

Other aspects of the present invention are directed to sub-genuses of compounds defined by each of Formulae I-a through I-k.

In some embodiments the present invention comprises a compound as defined in any embodiment described herein for use as a medicament.

The compounds of the present invention have utility in antiretroviral applications. Exemplary uses include anti-lentiviral applications, and anti-HIV applications. The treatment of HIV is a preferred use. All forms of HIV-1 are potentially treatable with compounds of the present invention. Compounds of the present invention have utility in treating protease inhibitor resistant HIV, reverse transcriptase inhibitor resistant HIV, and entry/fusion inhibitor resistant HIV. Compounds of the present invention have utility in treating HIV groups M, N, and O. Compounds of the present invention have utility in treating HIV-1, including subtypes A1, A2, B, C, D, F1, F2, G, H, J; and circulating recombinant HIV forms. Compounds of the present invention have utility in treating CCR5 tropic HIV strains as well as CXCR4 tropic HIV strains.

In some embodiments the present invention comprises the use of a compound as defined in any embodiment described herein, for the manufacture of a medicament to treat a disease for which an HIV inhibitor is desired.

In some embodiments the present invention comprises a method of preventing, treating or delaying the onset of AIDS in a subject in need thereof which comprises administering to the subject a therapeutically effective amount of a compound as defined in any embodiment described herein, optionally in combination with a therapeutically effective amount of at least one HIV inhibitor selected from the group consisting of HIV protease inhibitors, non-nucleoside HIV reverse transcriptase inhibitors, nucleoside HIV reverse transcriptase inhibitors, nucleotide HIV reverse transcriptase inhibitors, HIV maturation inhibitors, and HIV fusion inhibitors.

In some embodiments the present invention comprises a pharmaceutical composition which comprises the product prepared by combining an effective amount of (a) a compound as defined in any embodiment described herein, and (b) a pharmaceutically acceptable carrier.

### DETAILED DESCRIPTION OF THE INVENTION

Without wishing to be bound by theory, it is believed that the compounds of the present invention function by inhibiting fusion of the virion and cell membranes or entry of the viral core into the cellular cytoplasm, but not by inhibiting attachment of the virion to the cell.

Compounds of the present invention include compounds of Formula I:

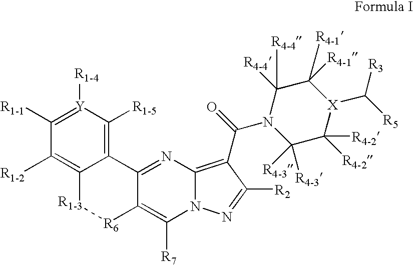

or a pharmaceutically acceptable salt or solvate thereof; wherein:

R₁₋₁ is selected from the group consisting of hydrido, hydroxyl, alkoxy, alkyl, cycloalkyl, dialkylamino, halo, haloalkyl, haloalkoxy, cyanoalkoxy, and nitro;

R₁₋₂ is selected from the group consisting of hydrido, hydroxyl, alkoxy, alkyl, and halo; or

R₁₋₁ and R₁₋₂ may be taken together in conjunction with the ring to which they are attached to form a heterocycle selected from the group consisting of 1,3-dioxolanyl, 1,4-dioxanyl, pyranyl, and 2,3-dihydrofuranyl;

R₁₋₃ is selected from the group consisting of hydrido, hydroxyl, alkoxy, alkyl, and halo when the dashed bond between R₁₋₃ and R₆ is not present or R₁₋₃ and R₆ are taken together to form (CHR₁₈)ₘ where m is 0, 1, or 2 when the dashed bond between R₁₋₃ and R₆ is present;

R₁₋₄ is selected from the group consisting of hydrido, hydroxyl, alkoxy, alkyl, and halo, with the proviso that R₁₋₄ is not present when Y is N;

R₁₋₅ is selected from the group consisting of hydrido, hydroxyl, alkoxy, alkyl, and halo;

R₂ is selected from the group consisting of hydrido, halo, hydroxyl, cyano, alkyl, alkoxy, alkoxyalkyl, alkoxyalkanoyl, alkoxycarbonyl, amido, alkylamino, alkylamido, alkylaminoalkyl, alkylamidoalkyl, cycloalkyl, hydroxyalkyl, aminoalkyl, alkylaminoalkyl, carboxyl, alkoxycarbonyl, aminocarbonyl, and arylalkyl;

R₃ is selected from the group consisting of alkyl, cycloalkyl, cyano, haloalkyl, hydroxyalkyl, alkoxyalkyl, aminoalkyl, alkylaminoalkyl, dialkylaminoalkyl, N-alkyl-N-alkenylaminoalkyl, carboxyl, alkoxycarbonyl, aminocarbonyl, alkylaminocarbonyl, dialkylaminocarbonyl, alkylcarbonylaminoalkyl, and alkylsulfonylaminoalkyl;

R₄₋₂′ and R₄₋₂″ are independently selected from the group consisting of hydrido, alkyl, cycloalkyl, hydroxyalkyl, alkoxyalkyl, aminoalkyl, alkylaminoalkyl, dialkylaminoalkyl, carboxyl, alkoxycarbonyl, aminocarbonyl, alkylaminocarbonyl, dialkylaminocarbonyl, arylalkyl, arylalkoxyalkyl, alkylaryl, haloarylalkyl, and haloalkylaryl; or R₄₋₂′ and R₄₋₂″ may be taken together to form a 3-8 membered carbocycle or a heterocycle;

R₄₋₄′ and R₄₋₄″ are independently selected from the group consisting of hydrido, alkyl, cycloalkyl, hydroxyalkyl, alkoxyalkyl, aminoalkyl, alkylaminoalkyl, dialkylaminoalkyl, carboxyl, alkoxycarbonyl, aminocarbonyl, alkylaminocarbonyl, dialkylaminocarbonyl, arylalkyl, alkylaryl, haloarylalkyl, and haloalkylaryl; or R₄₋₄′ and R₄₋₄″ may be taken together to form a 3-8 membered carbocycle or a heterocycle;

R₄₋₁′, R₄₋₁″, R₄₋₃′ and R₄₋₃″ are independently selected from the group consisting of hydrido, and alkyl, or R₄₋₁′, R₄₋₁″ may be taken together to form an oxo, or R₄₋₃′ and R₄₋₃″ may be taken together to form an oxo; or

any two of R₄₋₁′, R₄₋₁″, R₄₋₂′, R₄₋₂″, R₄₋₃′, R₄₋₃″, R₄₋₄′ and R₄₋₄″ may be taken together to form a 3-8 membered carbocycle or heterocycle;

R₅ is an optionally substituted C₆-C₁₀ aryl, or optionally substituted heteroaryl, wherein said heteroaryl comprises 1, 2, 3, or 4 heteroatoms independently selected from N, O and S;

R₆ is selected from the group consisting of hydrido, halo, alkyl, alkoxy, alkoxyalkyl, alkoxyalkanoyl, alkoxycarbonyl, amido, alkylamino, alkylamido, alkylaminoalkyl, alkylamidoalkyl, cycloalkyl, cyano, hydroxyl, hydroxyalkyl, aminoalkyl, alkylaminoalkyl, dialkylamino, dialkylaminoalkyl, carboxyl, alkoxycarbonyl, aminocarbonyl, arylalkyl, alkylaryl, haloarylalkyl, haloalkylaryl, alkylthio, alkylsulfonyl, and alkylsulfinyl when the dashed bond between R₁₋₃ and R₆ is not present, or R₁₋₃ and R₆ are taken together to form (CHR₁₈)ₘ where m is 0, 1, or 2 when the dashed bond between R₁₋₃ and R₆ is present;

R₇ is selected from the group consisting of hydrido, alkyl, alkoxy, alkoxyalkyl, alkoxyalkanoyl, alkoxycarbonyl, amido, alkylamino, alkylamido, alkylaminoalkyl, alkylamidoalkyl, cycloalkyl, hydroxyl, hydroxyalkyl, haloalkyl, aminoalkyl, alkylaminoalkyl, alkyl ester, carboxamido, dialkylamino, dialkylaminoalkyl, carboxyl, alkoxycarbonyl, aminocarbonyl, arylalkyl, alkylaryl, haloarylalkyl, haloalkylaryl, alkylthio, alkylsulfonyl, and alkylsulfinyl; and

R₁₈, in each instance, is independently selected from the group consisting of hydrido, alkyl, alkoxy, hydroxyl, and halo.

One subgenus of the present invention includes compounds of Formula I-a:

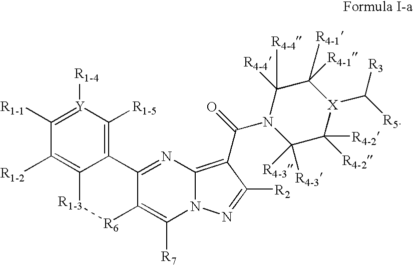

One subgenus of compounds of the present invention includes compounds according to the following formula where variable groups are as defined with respect to Formula I:

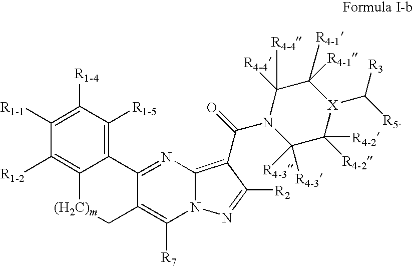

One subgenus of compounds of the present invention includes compounds according to the following formula where variable groups are as defined with respect to Formula I:

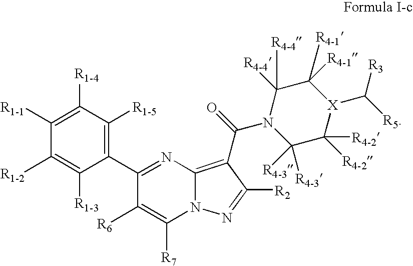

One subgenus of compounds of the present invention includes compounds according to the following formula where variable groups are as defined with respect to Formula I:

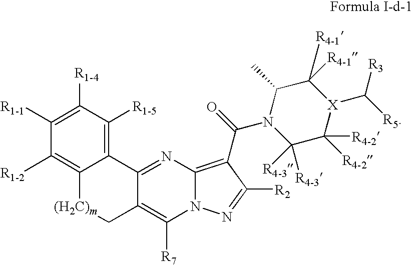

One subgenus of compounds of the present invention includes compounds according to the following formula where variable groups are as defined with respect to Formula I:

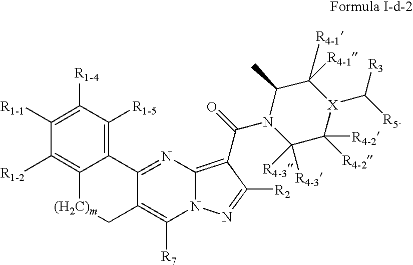

One subgenus of compounds of the present invention includes compounds according to the following formula where variable groups are as defined with respect to Formula I:

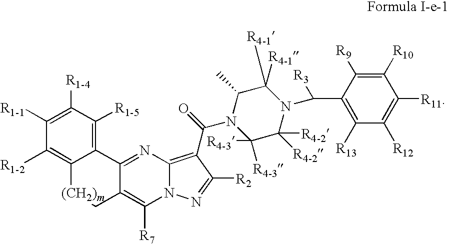

One subgenus of compounds of the present invention includes compounds according to the following formula where variable groups are as defined with respect to Formula I:

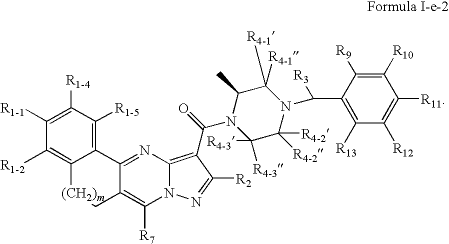

One subgenus of compounds of the present invention includes compounds according to the following formula where variable groups are as defined with respect to Formula I:

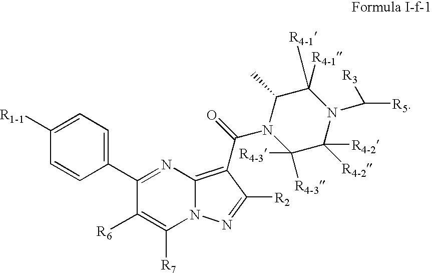

One subgenus of compounds of the present invention includes compounds according to the following formula where variable groups are as defined with respect to Formula I:

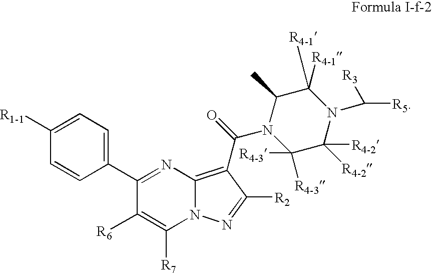

One subgenus of compounds of the present invention includes compounds according to the following formula where variable groups are as defined with respect to Formula I:

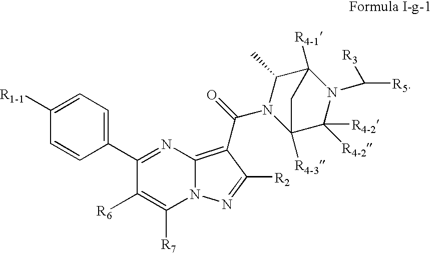

One subgenus of compounds of the present invention includes compounds according to the following formula where variable groups are as defined with respect to Formula I:

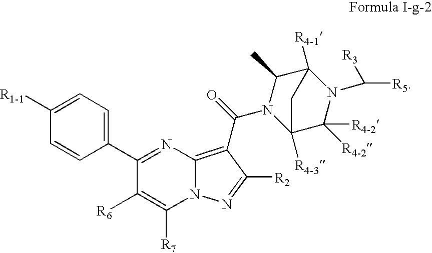

One subgenus of compounds of the present invention includes compounds according to the following formula where variable groups are as defined with respect to Formula I:

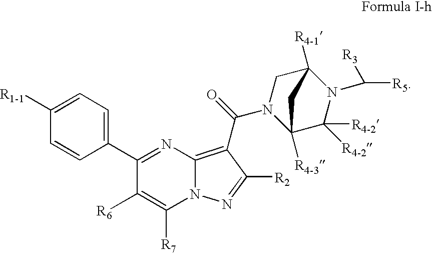

One subgenus of compounds of the present invention includes compounds according to the following formula where variable groups are as defined with respect to Formula I:

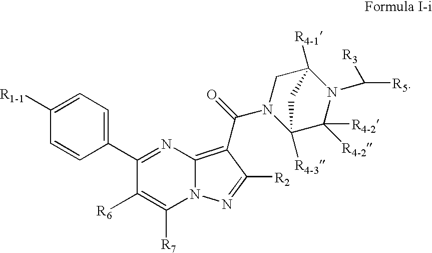

One subgenus of compounds of the present invention includes compounds according to the following formula where variable groups are as defined with respect to Formula I:

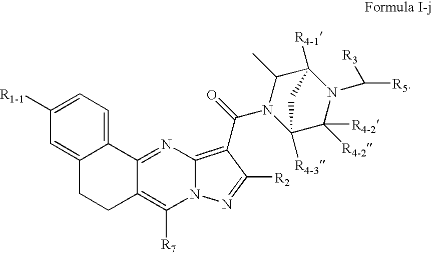

One subgenus of compounds of the present invention includes compounds according to the following formula where variable groups are as defined with respect to Formula I:

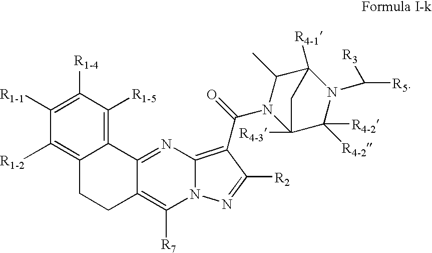

One embodiment is directed to a pharmaceutically acceptable salt or solvate of a compound of any of the above Formulae.

One embodiment is directed to compounds of the above Formulae, or a pharmaceutically acceptable salt or solvate thereof, wherein:

R₁₋₁ is selected from the group consisting of hydrido, hydroxyl, alkoxy, alkyl, cycloalkyl, dialkylamino, dimethylamino, halo, haloalkyl, haloalkoxy, cyanoalkoxy, and nitro;

R₁₋₂ is selected from the group consisting of hydrido, hydroxyl, alkoxy, alkyl, and halo:

R₁₋₃ is selected from the group consisting of hydrido, hydroxyl, alkoxy, alkyl, and halo;

R₁₋₄ is selected from the group consisting of hydrido, hydroxyl, alkoxy, alkyl, and halo;

R₁₋₅ is selected from the group consisting of hydrido, hydroxyl, alkoxy, alkyl, and halo;

R₂ is selected from the group consisting of hydrido, halo, hydroxyl, alkyl, and alkoxy;

R₃ is selected from the group consisting of alkyl, cycloalkyl, cyano, haloalkyl, hydroxyalkyl, alkoxyalkyl, aminoalkyl, alkylaminoalkyl, dialkylaminoalkyl, N-alkyl-N-alkenylaminoalkyl, carboxyl, alkoxycarbonyl, aminocarbonyl, alkylaminocarbonyl, dialkylaminocarbonyl, alkylcarbonylaminoalkyl, and alkylsulfonylaminoalkyl;

R₄₋₁′ and R₄₋₁″ are hydrido;

R₄₋₂′ and R₄₋₂″ are hydrido;

R₄₋₃′ and R₄₋₃″ are hydrido;

R₄₋₄′ and R₄₋₄″ are independently selected from the group consisting of hydrido, C₁-C₄ alkyl, cycloalkyl, C₁-C₄ hydroxyalkyl, or dialkylaminocarbonyl;

R₅ is selected from the group consisting of phenyl, naphthyl, pyridinyl, pyrazinyl, pyrimidinyl, furanyl, pyrrolyl, thienyl, pyrazolyl, imidazolyl, isoxazolyl, oxazolyl, thiazolyl, triazolyl, oxadiazolyl, thiadiazolyl, tetrazolyl, benzofuranyl, benzothienyl, indolyl, indazolyl, benzoxazolyl, benzimidazolyl, benzothiazolyl, benzoxazolyl, and benzodioxolanyl, any of which is optionally substituted with one or more groups independently selected from the group consisting of halo, cyano, nitro, alkyl, cycloalkyl, haloalkyl, hydroxyalkyl, alkoxyalkyl, aminoalkyl, monoalkylaminoalkyl, dialkylaminoalkyl, alkylcarbonylaminoalkyl, alkylsulfonylaminoalkyl, arylalkyl, formyl, alkanoyl, carboxyl, alkoxycarbonyl, alkoxyalkanoyl, aminocarbonyl, alkylaminocarbonyl, dialkylaminocarbonyl, amino, alkylamino, dialkylamino, alkanoylamino, alkylsulfonylamino, alkoxycarbonylamino, aminocarbonylamino, alkylaminocarbonylamino, dialkylaminocarbonylamino, azido, hydroxyl, alkoxy, haloalkoxy, alkylcarbonyloxy, aminocarbonyloxy, alkylaminocarbonyloxy, dialkylaminocarbonyloxy, amido, alkylamido, alkylamidoalkyl, alkylthio, alkylsulfinyl, alkylsulfonyl, aminosulfonyl, alkylaminosulfonyl, dialkylaminosulfonyl, pyrrolidinyl, piperidinyl, morpholinyl, piperazinyl, and 4-alkylpiperazinyl;

X is N;

Y is C;

R₆ is selected from the group consisting of hydrido, halo, such as fluoro, C₁-C₄ alkyl, such as methyl, or C₁-C₄ alkoxy, such as methoxy; and

R₇ is selected from the group consisting of hydrido, alkyl, alkoxy, alkoxyalkyl, alkoxyalkanoyl, alkoxycarbonyl, amido, alkylamino, alkylamido, alkylaminoalkyl, alkylamidoalkyl, cycloalkyl, hydroxyl, hydroxyalkyl, haloalkyl, aminoalkyl, alkylaminoalkyl, alkyl ester, carboxamido, dialkylamino, dialkylaminoalkyl, carboxyl, alkoxycarbonyl, aminocarbonyl, arylalkyl, alkylaryl, haloarylalkyl, haloalkylaryl, alkylthio, alkylsulfonyl, and alkylsulfinyl, preferably haloalkyl, such as difluoromethyl, trifluoromethyl or pentafluoroethyl, or dialkylamino, such as dimethylamino.

One embodiment is directed to compounds of the above Formulae, or a pharmaceutically acceptable salt or solvate thereof, wherein:

wherein R₁₋₁ is selected from the group consisting of hydrido, hydroxyl, alkoxy, alkyl, cycloalkyl, dialkylamino, halo, haloalkyl, haloalkoxy, cyanoalkoxy, and nitro;

R₁₋₂ is selected from the group consisting of hydrido, hydroxyl, alkoxy, alkyl, and halo, with the proviso that R₁₋₂ is hydrido when R₁₋₁ is hydrido;

or R₁₋₁ and R₁₋₂ may be taken together in conjunction with the ring to which they are attached to form a heterocycle selected from the group consisting of 1,3-dioxolanyl, 1,4-dioxanyl, pyranyl, and 2,3-dihydrofuranyl;

R₁₋₃ is selected from the group consisting of hydrido, hydroxyl, alkoxy, alkyl, and halo when the dashed bond between R₁₋₃ and R₆ is not present; or (CHR₁₈)ₘ where m is 0, 1, or 2 when the dashed bond between R₁₋₃ and R₆ is present;

R₁₋₄ is selected from the group consisting of hydrido, hydroxyl, alkoxy, alkyl, and halo, with the proviso that R₁₋₄ is not present when Y is N;

R₅ is selected from the group consisting of hydrido, hydroxyl, alkoxy, alkyl, and halo;

R₂ is selected from the group consisting of hydrido, hydroxyl, alkoxy, alkyl, and halo;

R₃ is selected from the group consisting of alkyl, haloalkyl, hydroxyalkyl, alkoxyalkyl, aminoalkyl, alkylaminoalkyl, dialkylaminoalkyl, carboxyl, alkoxycarbonyl, and aminocarbonyl;

R₄₋₂′ and R₄₋₂″ are independently selected from the group consisting of hydrido, alkyl, cycloalkyl, hydroxyalkyl, alkoxyalkyl, aminoalkyl, alkylaminoalkyl, dialkylaminoalkyl, carboxyl, alkoxycarbonyl, aminocarbonyl, arylalkyl, alkylaryl, haloarylalkyl, and haloalkylaryl, or R₄₋₂′ and R₄₋₂″ may be taken together to form an oxo, a 3-8 membered carbocycle, or a 3-8 membered heterocycle;

R₄₋₄′ and R₄₋₄″ are independently selected from the group consisting of hydrido, alkyl, cycloalkyl, hydroxyalkyl, alkoxyalkyl, aminoalkyl, alkylaminoalkyl, dialkylaminoalkyl, carboxyl, alkoxycarbonyl, aminocarbonyl, arylalkyl, alkylaryl, haloarylalkyl, and haloalkylaryl; or R₄₋₄′ and R₄₋₄″ may be taken together to form an oxo, a 3-8 membered carbocycle, or a 3-8 membered heterocycle;

R₄₋₁′, R₄₋₁″, R₄₋₃′ and R₄₋₃″ are independently selected from the group consisting of hydrido, and alkyl, or R₄₋₁′, R₄₋₁″ may be taken together to form an oxo, or R₄₋₃′ and R₄₋₃″ may be taken together to form an oxo, with the proviso that R₄₋₁′, R₄₋₁″, R₄₋₃′ and R₄₋₃″ may not be oxo when the alkylene bridge between C3 and C6 is present; or

any two of R₄₋₁′, R₄₋₁″, R₄₋₂′, R₄₋₂″, R₄₋₃′, R₄₋₃″, R₄₋₄′ and R₄₋₄″ may be taken together to form a 3-8 membered carbocycle or heterocycle;

R₅ is selected from the group consisting of a 5 membered ring comprising at least one unsaturation and 0, 1, or 2 heteroatoms selected from the group consisting of N, O, and S, isoxazolyl, α-naphthyl, 2-quinolinyl, hydroxyquinolinyl, 1,3-benzodioxolanyl, 1,4-dioxanyl, and pyranyl, wherein any of the above R₅ groups are optionally substituted with one or more moieties independently selected from the group consisting of halo, hydroxyl, alkoxy, and alkyl, or R₅ is

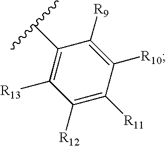

X is C or N;

Y is C or N;

R₆ is selected from the group consisting of hydrido, alkyl, cycloalkyl, halo, and cyano when the dashed bond between R₁₋₃ and R₆ is not present, or CHR₁₈ when dashed bond between R₁₋₃ and R₆ is present;

R₇ is selected from the group consisting of alkoxycarbonyl, alkylcarbonyloxy, alkyl, haloalkyl, amido, alkylamino, dialkylamino, halo, and cyano;

R₉, R₁₀, R₁₁, R₁₂ and R₁₃ are independently selected from the group consisting of hydrido, cyano, halo, alkoxy, alkyl, methylthio, azido, hydroxyl, amino, acetamido, methylsulfonylamino, trifluoromethyl, trifluoromethoxy, 1-pyrrolidinyl, cyclopropylcarbonylamino, acetyl, and methylsulfonyl; and

R₁₈ is hydrogen or alkyl.

The following embodiments apply to each of those formulae above individually that have the particular substitution patters as mentioned below.

In some embodiments R₁₋₁, R₁₋₂ and R₁₋₃ are hydrido.

In some embodiments R₁₋₁ is hydroxyl.

In some embodiments R₁₋₁ is alkoxy.

In some embodiments R₁₋₁ is methoxy.

In some embodiments R₁₋₁ is ethoxy.

In some embodiments R₁₋₁ is alkyl.

In some embodiments R₁₋₁ is methyl.

In some embodiments R₁₋₁ is cycloalkyl.

In some embodiments R₁₋₁ is cyclopropyl.

In some embodiments R₁₋₁ is dialkylamino.

In some embodiments R₁₋₁ is dimethylamino.

In some embodiments R₁₋₁ is halo.

In some embodiments R₁₋₁ is chloro.

In some embodiments R₁₋₁ is fluoro.

In some embodiments R₁₋₁ is haloalkyl.

In some embodiments R₁₋₁ is trifluoromethyl.

In some embodiments R₁₋₁ is nitro.

In some embodiments R₁₋₁ is methoxy and R₁₋₂ is hydroxyl.

In some embodiments R₁₋₁ is alkoxy and R₁₋₂ is alkoxy.

In some embodiments R₁₋₁ is methoxy and R₁₋₂ is methoxy.

In some embodiments R₁₋₁ is methoxy and R₁₋₂ is alkyl.

In some embodiments R₁₋₁ is methoxy and R₁₋₂ is methyl.

In some embodiments R₁₋₁ is methoxy and R₁₋₂ is halo.

In some embodiments R₁₋₁ is alkoxy and R₁₋₂, R₁₋₃, R₁₋₄ and R₁₋₅ are hydrido.

In some embodiments R₁₋₁ is methoxy and R₁₋₂, R₁₋₃, R₁₋₄, and R₁₋₅ are hydrido.

In some embodiments R₁₋₁ is alkoxy, R₁₋₂, R₁₋₄, and R₁₋₅ are hydrido, and R₁₋₃ and R₆ together form an ethano bridge.

In some embodiments R₁₋₁ is methoxy, R₁₋₂, R₁₋₄, and R₁₋₅ are hydrido, and R₁₋₃ and R₆ together form an ethano bridge.

In some embodiments R₁₋₁ is methoxy, R₁₋₂, R₁₋₄, and R₁₋₅ are hydrido, and R₁₋₃ and R₆ together form a propano bridge.

In some embodiments R₁₋₃ is alkoxy.

In some embodiments R₁₋₃ is methoxy.

In some embodiments R₁₋₃ is ethoxy.

In some embodiments R₁₋₃ is methyl.

In some embodiments R₁₋₃ is chloro.

In some embodiments R₁₋₃ is fluoro.

In some embodiments R₁₋₄ is methoxy, and Y is C.

In some embodiments R₁₋₄ is ethoxy, and Y is C.

In some embodiments R₁₋₄ is methyl, and Y is C.

In some embodiments R₁₋₄ is chloro, and Y is C.

In some embodiments R₁₋₄ is fluoro, and Y is C.

In some embodiments R₁₋₅ is methoxy.

In some embodiments R₁₋₅ is ethoxy.

In some embodiments R₁₋₅ is methyl.

In some embodiments R₁₋₅ is chloro.

In some embodiments R₁₋₅ is fluoro.

In some embodiments R₂ is hydrido.

In some embodiments R₂ is hydroxyl.

In some embodiments R₂ is alkoxy.

In some embodiments R₂ is alkyl.

In some embodiments R₂ is halo.

In some embodiments R₃ is alkyl.

In some embodiments R₃ is methyl.

In some embodiments R₃ is ethyl.

In some embodiments R₃ is cyclopropyl

In some embodiments R₃ is hydroxy.

In some embodiments R₃ is halo.

In some embodiments R₃ is haloalkyl, such as a monohaloalkyl, dihaloalkyl or trihaloalkyl, for example fluoromethyl, difluoromethyl or trifluoromethyl.

In some embodiments R₃ is hydroxyalkyl, such as hydroxymethyl, 1-hydroxyethyl, 2-hydroxyethyl or 1-hydroxy-1-methylethyl.

In some embodiments R₃ is alkoxyalkyl, such as methoxymethyl or ethoxymethyl.

In some embodiments R₃ is aminoalkyl, monoalkylaminoalkyl, dialkylaminoalkyl or N-alkyl-N-alkenylaminoalkyl, such as N-methylaminomethyl, N,N-dimethylaminomethyl or N-allyl-N-methylaminomethyl.

In some embodiments R₄₋₄′ is hydrido.

In some embodiments R₄₋₄′ is methyl.

In some embodiments R₄₋₄′ is (R) methyl.

In some embodiments R₄₋₄′ is (S) methyl.

In some embodiments R₄₋₄′ is dimethylaminocarbonyl.

In some embodiments R₄₋₄′ is hydroxymethyl.

In some embodiments R₄₋₄′ is (R) methyl, R₄₋₁ is oxo, and the dashed bond between C3 and C6 of the piperazine ring is not present.

In some embodiments R₄₋₄′ is (S) methyl, R₄₋₁ is oxo, and the dashed bond between C3 and C6 of the piperazine ring is not present.

In some embodiments R₄₋₄′ is (R) methyl, R₄₋₂′ and R₄₋₂″ together form oxo, and the dashed bond between C3 and C6 of the piperazine ring is not present.

In some embodiments R₄₋₄′ is (S) methyl, R₄₋₂′ and R₄₋₂″ together form oxo, and the dashed bond between C3 and C6 of the piperazine ring is not present.

In some embodiments R₄₋₄′ is (R) methyl, R₄₋₂′ and R₄₋₂″ together form oxo, and the dashed bond between C3 and C6 of the piperazine ring is present.

In some embodiments R₄₋₄′ is (S) methyl, R₄₋₂′ and R₄₋₂″ together form oxo, and the dashed bond between C3 and C6 of the piperazine ring is present.

In some embodiments R₅ is a 5 membered ring comprising at least one unsaturation and 1 heteroatom selected from the group consisting of N, O, and S.

Useful R₅ groups include phenyl, pyridinyl, pyrazinyl, pyrimidinyl, pyrimidinyl, furanyl, pyrrolyl, thienyl, pyrazolyl, imidazolyl, isoxazolyl, oxazolyl, thiazolyl, triazolyl, oxadiazolyl, thiadiazolyl, tetrazolyl, benzofuranyl, benzothienyl, indolyl, indazolyl, benzoxazolyl, benzimidazolyl, benzothiazolyl, benzoxazolyl, and benzodioxolanyl, any of which is optionally substituted with one or more groups independently selected from the group consisting of halo, cyano, nitro, alkyl, cycloalkyl, haloalkyl, hydroxyalkyl, alkoxyalkyl, aminoalkyl, alkylaminoalkyl, dialkylaminoalkyl, alkylcarbonylaminoalkyl, alkylsulfonylaminoalkyl, arylalkyl, formyl, alkanoyl, carboxyl, alkoxycarbonyl, alkoxyalkanoyl, aminocarbonyl, alkylaminocarbonyl, dialkylaminocarbonyl, amino, alkylamino, dialkylamino, alkanoylamino, alkylsulfonylamino, alkoxycarbonylamino, aminocarbonylamino, alkylaminocarbonylamino, dialkylaminocarbonylamino, azido, hydroxyl, alkoxy, haloalkoxy, alkylcarbonyloxy, aminocarbonyloxy, alkylaminocarbonyloxy, dialkylaminocarbonyloxy, amido, alkylamido, alkylamidoalkyl, alkylthio, alkylsulfinyl, alkylsulfonyl, aminosulfonyl, alkylaminosulfonyl, dialkylaminosulfonyl, pyrrolidinyl, piperidinyl, morpholinyl, piperazinyl, and 4-alkylpiperazinyl.

In one embodiment, R₅ is selected from the group consisting of a 5 membered ring comprising at least one unsaturation and 0, 1, or 2 heteroatoms selected from the group consisting of N, O, and S, α-naphthyl, 2-quinolinyl, hydroxyquinolinyl, 1,3-benzodioxolanyl, 1,4-benzodioxanyl, and benzopyranyl, where R₅ is optionally substituted with one or more moieties independently selected from the group consisting of halo, hydroxyl, alkoxy, and alkyl.

In some embodiments R₅ is optionally substituted thienyl.

In some embodiments R₅ is alkylthienyl.

In some embodiments R₅ is optionally substituted furanyl.

In some embodiments R₅ is optionally substituted pyrrolyl.

In some embodiments R₅ is α-naphthyl.

In some embodiments R₅ is optionally substituted quinolin-2-yl.

In some embodiments R₅ is hydroxyquinolinyl.

In some embodiments R₅ is 8-hydroxyquinolin-2-yl.

In some embodiments R₅ is 1,3-benzodioxolanyl.

In some embodiments R₅ is optionally substituted pyridyl.

In some embodiments R₅ is optionally substituted pyrimidinyl.

In some embodiments R₅ is optionally substituted pyridazinyl.

In some embodiments R₅ is optionally substituted pyrazolyl.

In some embodiments R₅ is optionally substituted isoxazolyl.

In some embodiments R₅ is optionally substituted oxazolyl.

In some embodiments R₅ is optionally substituted benzofuranyl.

In some embodiments R₅ is optionally substituted indolyl.

In another embodiment, R₅ is Formula R:

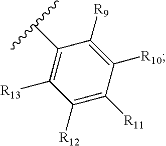

where R₉, R₁₀, R₁₁, R₁₂ and R₁₃ are independently selected from the group consisting of hydrido, cyano, halo, alkoxy, alkyl, methylthio, azido, and hydroxyl. In a preferred embodiment, at least one of R₉, R₁₀, R₁₁, R₁₂ and R₁₃ is not hydrido.

In some embodiments R₅ is Formula R where one of R₉, R₁₀, R₁₁, R₁₂ and R₁₃ is hydroxy.

In some embodiments R₅ is Formula R where one of R₉, R₁₀, R₁₁, R₁₂ and R₁₃ is halo.

In some embodiments R₅ is Formula R where one of R₉, R₁₀, R₁₁, R₁₂ and R₁₃ is fluoro.

In some embodiments R₅ is Formula R where R₉ is fluoro or chloro.

In some embodiments R₅ is Formula R where R₁₀ is fluoro or chloro.

In some embodiments R₅ is Formula R where R₁₁ is fluoro or chloro.

In some embodiments R₅ is Formula R where R₁₂ is fluoro or chloro.

In some embodiments R₅ is Formula R where R₁₃ is fluoro or chloro.

In some embodiments R₅ is Formula R where two of R₉, R₁₀, R₁₁, R₁₂ and R₁₃ are halo.

In some embodiments R₅ is Formula R where two of R₉, R₁₀, R₁₁, R₁₂ and R₁₃ are fluoro.

In some embodiments R₅ is Formula R where R₉ and R₁₀ are fluoro.

In some embodiments R₅ is Formula R where R₉ and R₁₁ are fluoro.

In some embodiments R₅ is Formula R where R₉ and R₁₂ are fluoro.

In some embodiments R₅ is Formula R where R₁₁ and R₁₂ are fluoro.

In some embodiments R₅ is Formula R where three of R₉, R₁₀, R₁₁, R₁₂ and R₁₃ are halo.

In some embodiments R₅ is Formula R where three of R₉, R₁₀, R₁₁, R₁₂ and R₁₃ are fluoro.

In some embodiments R₅ is Formula R where R₉, R₁₀, and R₁₂ are fluoro.

In some embodiments R₅ is Formula R where four of R₉, R₁₀, R₁₁, R₁₂ and R₁₃ are fluoro.

In some embodiments R₅ is Formula R where R₉, R₁₀, R₁₁, R₁₂ and R₁₃ are fluoro.

In some embodiments R₇ is alkoxycarbonyl.

In some embodiments R₇ is methoxycarbonyl.

In some embodiments R₇ is alkyl.

In some embodiments R₇ is methyl.

In some embodiments R₇ is ethyl.

In some embodiments R₇ is cyclopropyl.

In some embodiments R₇ is haloalkyl.

In some embodiments R₇ is trifluoromethyl.

In some embodiments R₇ is difluoromethyl

In some embodiments R₇ is amido.

In some embodiments R₇ is alkylamino.

In some embodiments R₇ is methylamino.

In some embodiments R₇ is dialkylamino.

In some embodiments R₇ is dimethylamino.

In some embodiments R₇ is halo.

In some embodiments R₇ is fluoro.

In some embodiments R₇ is chloro.

In some embodiments R₇ is cyano.

In some embodiments R₆ is hydrido.

In some embodiments R₆ is alkyl.

In some embodiments R₆ is methyl.

In some embodiments R₆ is fluoro.

In some embodiments R₆ is alkoxy.

In some embodiments R₆ is methoxy.

In some embodiments the present invention comprises a compound as defined in any embodiment described herein for use as a medicament.

In some embodiments the present invention comprises the use of a compound as defined in any embodiment described herein, for the manufacture of a medicament to treat a disease for which an HIV inhibitor is desired.

In some embodiments the present invention comprises a method of preventing, treating or delaying the onset of AIDS in a subject in need thereof which comprises administering to the subject a therapeutically effective amount of a compound as defined in any embodiment described herein, optionally in combination with a therapeutically effective amount of at least one HIV inhibitor selected from the group consisting of HIV protease inhibitors, non-nucleoside HIV reverse transcriptase inhibitors, nucleoside HIV reverse transcriptase inhibitors, nucleotide HIV reverse transcriptase inhibitors, HIV maturation inhibitors, and HIV fusion inhibitors. In some embodiments the present invention comprises a pharmaceutical composition which comprises the product prepared by combining an effective amount of (a) a compound as defined in any embodiment described herein, and (b) a pharmaceutically acceptable carrier.

The compounds of the present invention have utility in antiretroviral applications. Exemplary uses include anti-lentiviral applications, and anti-HIV applications. The treatment of HIV is a preferred use. All forms of HIV-1 are potentially treatable with compounds of the present invention. Compounds of the present invention have utility in treating protease inhibitor resistant HIV, reverse transcriptase inhibitor resistant HIV, and entry/fusion inhibitor resistant HIV. Compounds of the present invention have utility in treating HIV groups M, N, and O. Compounds of the present invention have utility in treating HIV-1, including subtypes A1, A2, B, C, D, F1, F2, G, H, J; and circulating recombinant HIV forms. Compounds of the present invention have utility in treating CCR5 tropic HIV strains as well as CXCR4 tropic HIV strains.

The compounds of the present invention differ from the referenced background compounds in structure, pharmacological activity, or pharmacological potency. Some compounds of the invention not only act favorably in terms of their capability to inhibit the replication of HIV-1, but also by their improved ability to inhibit the replication of mutant strains, in particular strains which have become resistant to commercially available drugs.

Some compounds of the present invention have utility in antidiabetic applications. Compounds of the present invention have utility in treating diabetes by mediating 11β-hydroxysteroid dehydrogenase type 1 (“11βHSD-1”).

The term “Ac” means acetyl.

The term “alkyl”, as used alone or within other terms such as “haloalkyl” and “alkylsulfonyl”, means an acyclic alkyl radical, linear or branched, preferably containing from 1 to about 10 carbon atoms and more preferably containing from 1 to about 6 carbon atoms. “Alkyl” also encompasses the sub-genera of alkenes and alkynes, such as ethenyl, ethynyl, propenyl, propynyl, isopropyl, isopropenyl, and other linear, branched or cyclic structures having 2-10 carbon atoms. “Alkyl” also encompasses the sub-genus of cyclic alkyl radicals containing from 3 to about 7 carbon atoms, preferably from 3 to 5 carbon atoms. Said alkyl radicals can be optionally substituted with groups as defined below. Examples of such radicals include methyl, ethyl, chloroethyl, hydroxyethyl, n-propyl, isopropyl, n-butyl, cyanobutyl, isobutyl, sec-butyl, tert-butyl, pentyl, aminopentyl, isoamyl, hexyl, octyl, cyclopropyl, cyclohexyl, cyclohexenyl, and propynyl.

The term “alkoxy” embraces linear or branched oxy-containing radicals each having alkyl portions of 1 to about 6 carbon atoms, preferably 1 to about 3 carbon atoms, such as a methoxy radical. The term “alkoxyalkyl” also embraces alkyl radicals having one or more alkoxy radicals attached to the alkyl radical, that is, to form monoalkoxyalkyl and dialkoxyalkyl radicals. Examples of such radicals include methoxy, ethoxy, propoxy, butoxy and tert-butoxy alkyls. The “alkoxy” radicals may be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylthio” embraces radicals containing a linear or branched alkyl radical, of 1 to about 6 carbon atoms, attached to a divalent sulfur atom. An example of lower alkylthio is methylthio (CH₃S).

The term “alkylthioalkyl” embraces alkylthio radicals, attached to an alkyl group. An example of alkylthioalkyl is methylthiomethyl.

The term “Alloc Cl” refers to allyl chloroformate. The term “Alloc” refers to the allyloxycarbonyl portion of this molecule.

The terms “amido” when used independently or in conjunction with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces carbonylamino radicals including radicals where the nitrogen is covalently bonded to 2 hydrogens, to 1 hydrogen and 1 atom other than hydrogen, and to 2 atoms other than hydrogen.

The term “amu” means atomic mass unit.

The term “app” means apparent in reference to spectral data.

The term “aryl” means a fully unsaturated mono- or multi-ring carbocycle. Examples of such radicals include substituted or unsubstituted phenyls, naphthyls, and anthracenyls. The term “aryl”, as used alone or within other terms, means a mono- or multi-ring aromatic ring structure containing between one and four rings wherein such rings may be attached together in a pendent manner or may be fused. Such an “aryl” group may have 1 or more substituents such as lower alkyl, hydroxy, halo, haloalkyl, nitro, cyano, alkoxy and lower alkylamino. The term “aryl” refers to both cyclic structures consisting only of carbon (carboaryls), and cyclic structures comprising carbon and one or more heteroatoms selected from the group consisting of nitrogen, sulfur and oxygen (heteroaryls).

The terms “t-Boc”, “BOC” and “Boc” means tert-butoxycarbonyl.

The term “Boc-ON” means 2-(tert-butoxycarbonyloxyimino)-2-phenylacetonitrile.

The term “br” in reference to spectral data means broad.

The term “n-Bu” means linear butyl.

The term “t-Bu” means tert-butyl.

The term “Bzl” means benzyl.

The term “C” means degrees Celsius.

The term “cat” means catalytic.

The term “CDI” means 1,1′-carbonyldiimidazole.

The term “conc.” or “concd” means concentrated.

The term “carbocycle” as used alone or within other terms, means a mono- or multi-ring ring structure consisting only of carbon containing between one and four rings wherein such rings may be attached together in a pendent manner or may be fused. The term “carbocycle” refers to fully saturated and unsaturated ring systems as well as partially unsaturated ring systems. The term “carbocycle” additionally encompasses spiro systems wherein one cycloalkyl ring has a carbon ring atom in common with another cycloalkyl ring. The term “carbocycle” additionally encompasses bridged systems. Illustrative examples of monocyclic, bicyclic or tricyclic saturated carbocycles include cyclopropyl, cyclobutyl, cyclopentyl, cyclohexyl, cycloheptyl, cyclooctyl, bicyclo[4.2.0]octanyl, bicyclo[2.2.1]heptanyl, bicyclo[2.2.2]octanyl, cyclononanyl, cyclodecanyl, decahydronapthalenyl, and tetradecahydroanthracenyl. Illustrative examples of monocyclic, bicyclic or tricyclic partially saturated carbocycles include cyclopropenyl, cyclobutenyl, cyclopentenyl, cyclohexenyl, cycloheptenyl, cyclooctenyl, bicyclo[2.2.1]heptenyl, bicyclo[2.2.2]octenyl, bicyclo[4.2.0]octenyl, cyclononenyl, cyclodecenyl, octahydronaphthalenyl, 1,2,3,4-tetrahydronaphthalenyl, and 1,2,3,4,4a,9,9a,10-octahydroanthracenyl. Illustrative examples of monocyclic, bicyclic or tricyclic aromatic carbocycles include phenyl, naphthalenyl, and anthracenyl. Thus, the term “carbocycle” includes the following exemplary structures:

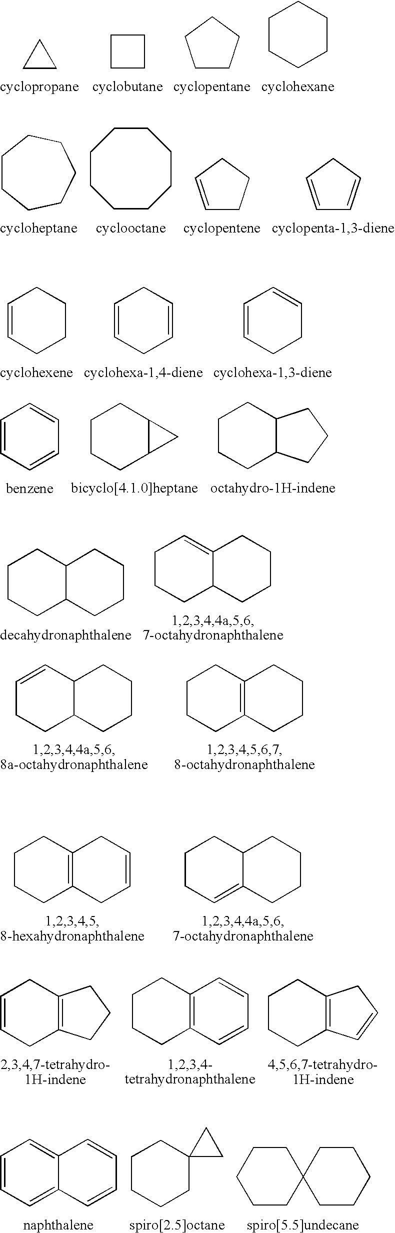

The terms “carboxy” and “carboxyl”, whether used alone or with other terms, such as “carboxyalkyl”, denotes —CO₂H.

The term “combination therapy” refers to the administration of a compound of the present invention and a secondary anti-infective or pharmaceutical agent as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. For example, one combination of the present invention comprises a reverse transcriptase inhibitor and a fusion inhibitor of the present invention administered as separate agents at the same or different times or they can be formulated as a single, co-formulated pharmaceutical composition comprising the two compounds. As another example, a combination of the present invention comprises a reverse transcriptase inhibitor and a fusion inhibitor of the present invention formulated as separate pharmaceutical compositions that can be administered at the same or different time. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, one component of a particular combination may be administered by intravenous injection while the other component(s) of the combination may be administered orally. The components may be administered in any therapeutically effective sequence.

The term “δ” means chemical shift in parts per million downfield from tetramethylsilane.

The term “d” in reference to time means days; the term “d” in reference to spectral data means doublet.

The term “an” means density.

The term “de” means diastereomeric excess.

The term “DCC” means N,N-dicyclohexylcarbodiimide.

The term “DCE” means 1,2-dichloroethane.

The term “DCM” means dichloromethane.

The term “DIAD” means diisopropyl azodicarboxylate.

The term “DIP-Cl” means chlorodiisopinocampheylborane.

The term “DIPEA” means N,N-diisopropylethylamine.

The term “DMAP” means 4-N,N-dimethylaminopyridine.

The term “DME” means 1,2-dimethoxyethane.

The term “DMF” means N,N-dimethylformamide.

The term “DMSO” means dimethyl sulfoxide.

The term “dppf” means 1,1′-bis(diphenylphosphino)ferrocene.

The term “EC₅₀” means the drug concentration that results in a 50% reduction in virus replication.

The term “EDC” means 1-(3-dimethylaminopropyl)-3-ethylcarbodiimide hydrochloride.

The term “ee” means enantiomeric excess.

The term “equiv” or “eq” means equivalents.

The term “ES” means electrospray ionization.

The term “Et” means ethyl.

The term “g” means grams.

The term “h” or “hr” means hours.

The term “halo” means a halogen radical derived from fluorine, chlorine, bromine or iodine. The term “haloalkyl” embraces radicals wherein any one or more of the alkyl carbon atoms is substituted with halo as defined above. Specifically embraced are monohaloalkyl, polyhaloalkyl, and perhalo radicals. A monohaloalkyl radical, for one example, may have one atom selected from the group consisting of iodo, bromo, chloro and fluoro atoms within the radical. Polyhaloalkyl radicals may have two or more of the same halo atoms or a combination of different halo radicals. “Lower haloalkyl” embraces radicals having 1-6 carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perfluoroalkyl” means an alkyl radical having all hydrido radicals replaced with fluorine atoms. Examples include trifluoromethyl and pentafluoroethyl.

The term “HATU” means O-(7-azabenzotriazol-1-yl)-N,N,N′,N′-tetramethyluronium hexafluorophosphate.

The term “heterocyclyl” means a saturated or unsaturated mono- or multi-ring carbocycle wherein one or more carbon atoms are replaced by N, S, P, or O. The term “heterocycle” refers to fully saturated and unsaturated ring systems as well as partially unsaturated ring systems. The term “heterocycle” is intended to include all the possible isomeric forms of the heterocycle, for example, pyrrolyl comprises 1H-pyrrolyl and 2H-pyrrolyl. Illustrative examples of monocyclic, bicyclic or tricyclic saturated heterocycles include tetrahydrofuranyl, pyrrolidinyl, dioxolanyl, imidazolidinyl, thiazolidinyl, tetrahydrothienyl, dihydrooxazolyl, isothiazolidinyl, isoxazolidinyl, oxadiazolidinyl, triazolidinyl, thiadiazolidinyl, pyrazolidinyl, piperidinyl, hexahydropyrimidinyl, hexahydropyrazinyl, dioxanyl, morpholinyl, dithianyl, thiomorpholinyl, piperazinyl, trithianyl, decahydroquinolinyl, and octahydroindolyl. Illustrative examples of monocyclic, bicyclic or tricyclic partially saturated heterocycles include azetyl, pyrrolinyl, imidazolinyl, pyrazolinyl, 2,3-dihydrobenzofuranyl, 1,3-benzodioxolanyl, 2,3-dihydro-1,4-benzodioxinyl, indolinyl and the like. Illustrative examples of monocyclic, bicyclic or tricyclic aromatic heterocycles include pyrrolyl, furanyl, thienyl, imidazolyl, oxazolyl, isoxazolyl, thiazolyl, isothiazolyl, pyrazolyl, triazolyl, thiadiazolyl, oxadiazolyl, tetrazolyl, pyridinyl, pyrimidinyl, pyrazinyl, pyridazinyl, triazinyl, pyranyl, benzofuranyl, isobenzofuranyl, benzothienyl, isobenzothienyl, indolizinyl, indolyl, isoindolyl, benzoxazolyl, benzimidazolyl, indazolyl, benzisoxazolyl, benzisothiazolyl, benzopyrazolyl, benzoxadiazolyl, benzothiadiazolyl, benzotriazolyl, purinyl, quinolinyl, isoquinolinyl, cinnolinyl, quinolizinyl, phthalazinyl, quinoxalinyl, quinazolinyl, naphthyridinyl, pteridinyl, benzopyranyl, pyrrolopyridinyl, thienopyridinyl, furanopyridinyl, isothiazolopyridinyl, thiazolopyridinyl, isoxazolopyridinyl, oxazolopyridinyl, pyrazolopyridinyl, imidazopyridinyl, pyrrolopyrazinyl, thienopyrazinyl, furanopyrazinyl, isothiazolopyrazinyl, thiazolopyrazinyl, isoxazolopyrazinyl, oxazolopyrazinyl, pyrazolopyrazinyl, imidazopyrazinyl, pyrrolopyrimidinyl, thienopyrimidinyl, furanopyrimidinyl, isothiazolopyrimidinyl, thiazolopyrimidinyl, isoxazolopyrimidinyl, oxazolopyrimidinyl, pyrazolopyrimidinyl, imidazopyrimidinyl, pyrrolopyridazinyl, thienopyridazinyl, furanopyridazinyl, isothiazolopyridazinyl, thiazolopyridazinyl, isoxazolopyridazinyl, oxazolopyridazinyl, pyrazolopyridazinyl, imidazopyridazinyl, oxadiazolopyridinyl, thiadiazolopyridinyl, triazolopyridinyl, oxadiazolopyrazinyl, thiadiazolopyrazinyl, triazolopyrazinyl, oxadiazolopyrimidinyl, thiadiazolopyrimidinyl, triazolopyrimidinyl, oxadiazolopyridazinyl, thiadiazolopyridazinyl, triazolopyridazinyl, imidazooxazolyl, imidazothiazolyl, imidazoimidazolyl, isoxazolotriazinyl, isothiazolotriazinyl, pyrazolotriazinyl, oxazolotriazinyl, thiazolotriazinyl, imidazotriazinyl, oxadiazolotriazinyl, thiadiazolotriazinyl, triazolotriazinyl, carbazolyl, acridinyl, phenazinyl, phenothiazinyl, and phenoxazinyl. Thus, the term “heterocycle” includes the following exemplary structures which are not depicted as radicals as each may form be attached through a covalent bond to any atom so long as appropriate valences are maintained:

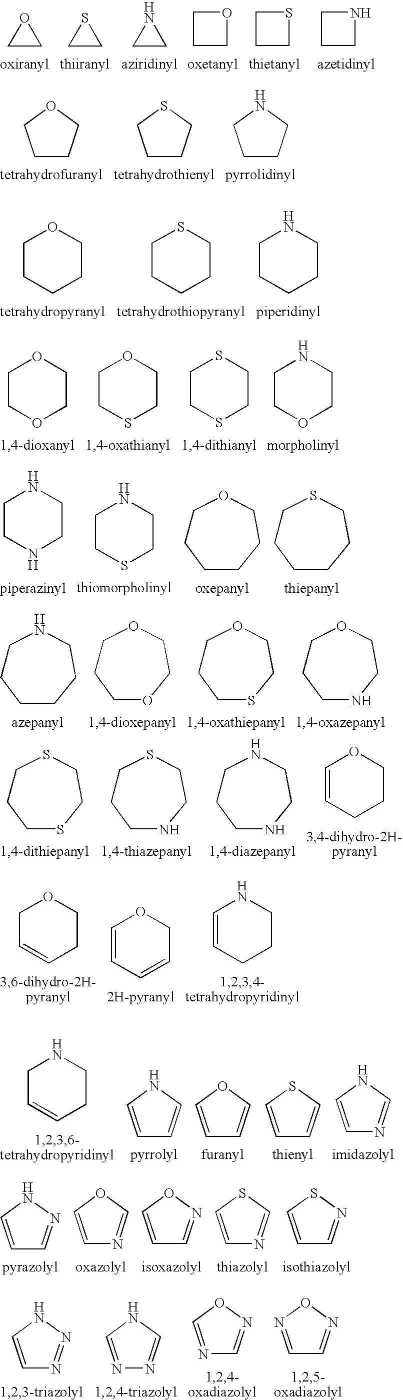

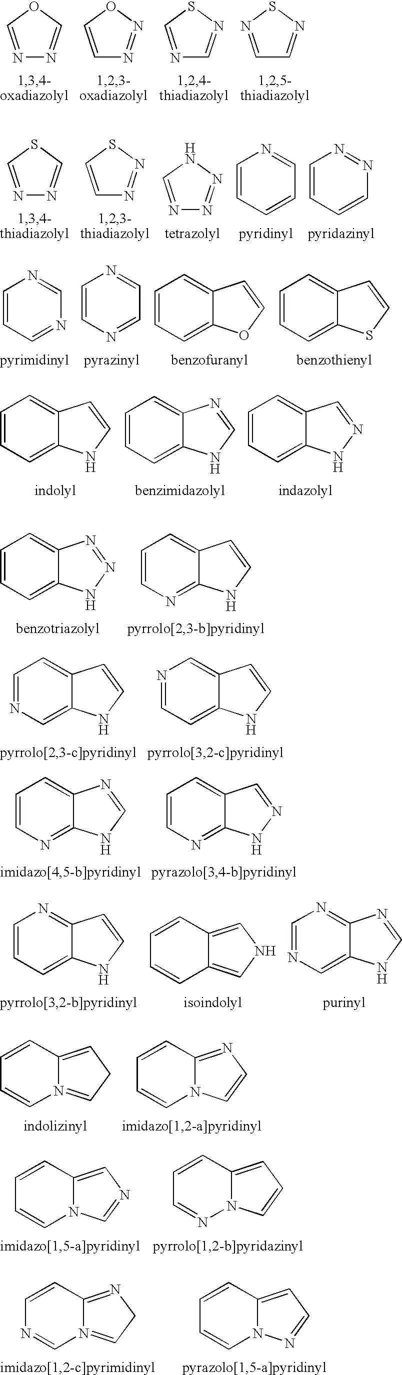

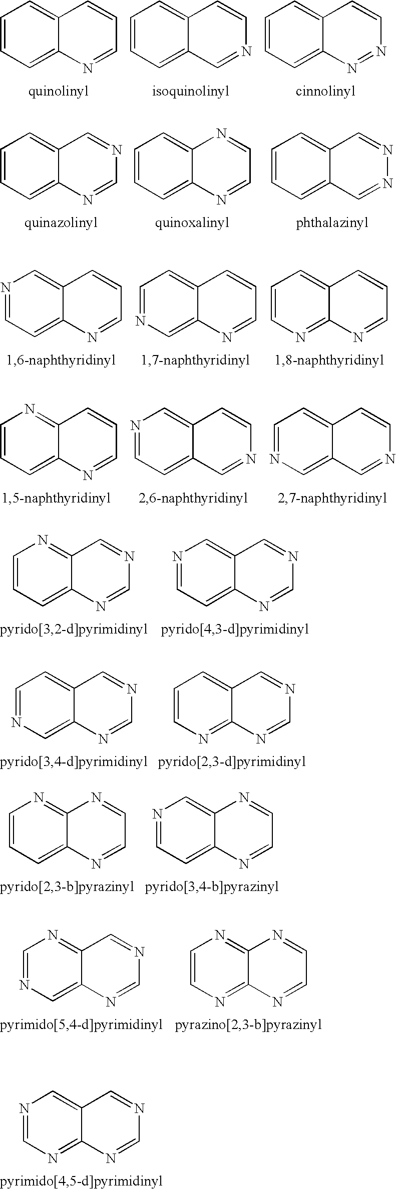

The term “heteroaryl” means a fully unsaturated heterocycle.

With regard to any of “carbocycle,” “aryl,” “heterocycle,” or “heteroaryl”, the point of attachment to the molecule of interest can be at the heteroatom or elsewhere within the ring. For terms such as aralkyl, and heteroarylalkyl, the moiety may be linked through any ring atom or through any atom of the alkyl portion so long as the resultant molecule is chemically stable. The presence of charge, for example when a pyridinyl radical is attached via the ring nitrogen to yield a quaternary nitrogen, does not in and of itself mean that the resultant molecule is not chemically stable. The use of “carbocycle,” “aryl,” “heterocycle,” and “heteroaryl” moieties includes divalent attachment at appropriate substitutable sites.

The term “HOBT” means 1-hydroxybenzotriazole.

The term “HPLC” means high performance liquid chromatography.

The term “hydrido” means a single hydrogen atom (H). This hydrido radical may be attached, for example, to an oxygen atom to form a hydroxyl radical (—OH) or two hydrido radicals may be attached to a carbon atom to form a methylene (—CH₂—) radical.

The term “Hz” means hertz.

Depending on context, the term “IC₅₀” means either the drug concentration that results in inhibition of 50% of virus replication when referring to virus replication assays, or the drug concentration that results in inhibition of 50% of 6HB formation when referring to the 6HB assay.

The term “L” means liters.

The term “LAH” means lithium aluminum hydride.

The term “LC” means liquid chromatography.

The term “LHMDS” means lithium hexamethyldisilazide.

The term “μ” means 10⁻⁶.

The term “m” in reference to an amount means 10⁻³; the term “m” in reference to a spectral data means multiplet.

The term “M” means molar.

The term “Me” means methyl.

The term “min” means minutes.

The term “mol” means moles.

The term “MS” means mass spectrometry.

The term “Ms” means mesyl or methanesulfonyl.

The term “MT-2 cells” refers to human T-cell leukemia cells isolated from cord blood lymphocytes and co-cultured with cells from patients with adult T-cell leukemia. The MT-2 cell line was acquired from the AIDS Research and Reference Reagent Program.

The term “MTBE” means methyl tert-butyl ether.

The term “m/z” means mass-to-charge ratio.

The term “NMP” means N-methylpyrrolidinone.

The term “NMR” means nuclear magnetic resonance.

The term “obs” in reference to spectral data means obscured.

The term “oxo” means a doubly bonded oxygen.

The term “Ph” means phenyl.

The term “prodrug” means a chemical derivative of an active parent drug that requires upon spontaneous or enzymatic biotransformation releasing the active parent drug. The term “prodrug” includes variations or derivatives of the compounds of this invention which have groups cleavable under metabolic conditions including solvolysis or enzymatic degradation. In some embodiments of the present invention the prodrug is either pharmacologically inactive or exhibits reduced activity relevant to its active parent drug.

The term “q” in reference to spectral data means quartet.

The term “rt” means room temperature.

The term “s” in reference to spectral data means singlet.

The term “satd” means saturated.

The term “selective” as referring to a particular event means that the particular event occurs with greater frequency than other potential event(s).

The term “solvate” means a molecular complex comprising a compound of the present invention and a proportional number of solvent molecules. The term “hydrate” means a solvate where the solvent is water. In some embodiments of the present invention the solvate comprises a fractional amount of a solvent molecule per molecule of the present invention, for example, a hemisolvate. In some embodiments of the present invention the solvate comprises one solvent molecule per molecule of the present invention, for example, a monosolvate. In some embodiments of the present invention the solvate comprises two solvent molecules per molecule of the present invention, for example, a disolvate.

The term “STAB” means sodium triacetoxyborohydride.

The term “t” in reference to spectral data means triplet.

The term “Tf” means trifluoromethanesulfonyl.

The term “TBDMS” means t-butyldimethylsilyl.

The term “TEA” means triethylamine.

The term “TEOF” means triethylorthoformate.

The term “TFA” means trifluoroacetic acid.

“Therapeutic effect” as used herein means some extent of relief of one or more of the symptoms of an HIV-related disorder. In reference to the treatment of HIV, a therapeutic effect refers to one or more of the following: 1) reduction in the number of infected cells; 2) reduction in the number of virions present in serum; 3) inhibition (i.e., slowing to some extent, preferably stopping) the rate of HIV replication; 6) relieving or reducing to some extent one or more of the symptoms associated with HIV; and 7) relieving or reducing the side effects associated with the administration of other antiretroviral agents.

“Therapeutically effective amount” as used herein means the amount required to achieve a therapeutic effect.

The term “THF” means tetrahydrofuran.

The term “TI” means the CC₅₀:EC₅₀ ratio of a compound.

The term “TLC” means thin layer chromatography.

The term “TMS” means trimethylsilyl.

The term “t_{R}” in reference to chromatographic analysis means retention time.

The term “Ts” means p-toluenesulfonyl.

“Weight percent” as used herein means the weight percent of a specified ingredient based upon the total weight of all ingredients of the composition.

### EXAMPLES

### Representative Species

The following species comprise some representative species of Formula I. Note that the numbering of the representative species is independent of the numbering used in the working examples. For both 6HB (six-helix bundle formation) and virus entry (infection) assays, activity profiles for compounds having or exhibiting an IC₅₀ less than 1.0 μM are accorded a “+++” designation; activity profiles for compounds having or exhibiting an IC₅₀ between 1.1 μM and 10.0 μM are accorded a “++” designation; and activity profiles for compounds having or exhibiting an IC₅₀ greater than 10.1 μM are accorded a “+” designation. The following embodiments are illustrative of the claimed invention and are not intended to limit the scope of the present invention to the embodiments listed below. “ND” means not determined. A number of the following compounds were found to be extremely potent, exhibiting IC₅₀ values in the picomolar range in both assay formats.

| | |
|---|---|
| Structure | 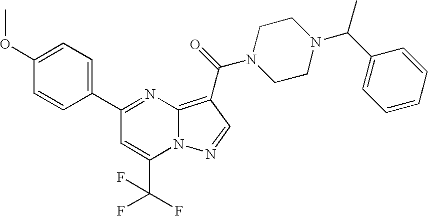 |
| Molecular Weight | 509.52 |
| Name | methanone, [5-(4-methoxyphenyl)-7- |
| | (trifluoromethyl)pyrazolo[1,5-α]pyrimidin-3-yl][4-(1- |
| | phenylethyl)-1-piperazinyl]- |
| 6HB IC50 | ++ |
| Nl4 | ND |
| Structure | 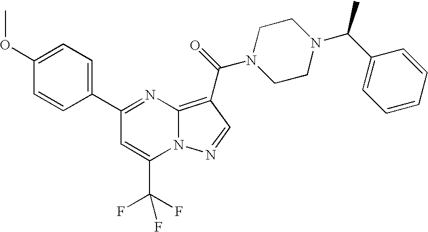 |
| Molecular Weight | 509.52 |
| Name | methanone, [5-(4-methoxyphenyl)-7- |
| | (trifluoromethyl)pyrazolo[1,5-α]pyrimidin-3-yl][4-(1- |
| | phenylethyl)-1 -piperazinyl]- |
| 6HB IC50 | ++ |
| Nl4 | ND |
| Structure | 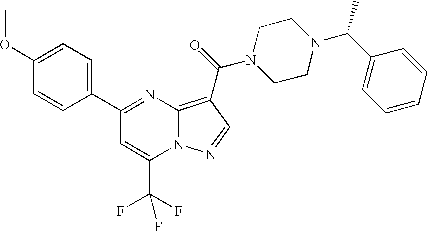 |
| Molecular Weight | 509.52 |
| Name | methanone, [5-(4-methoxyphenyl)-7- |
| | (trifluoromethyl)pyrazolo[1,5-α]pyrimidin-3-yl][4-(1- |
| | phenylethyl)-1-piperazinyl]- |
| 6HB IC50 | ++ |
| Nl4 | ND |
| Structure | 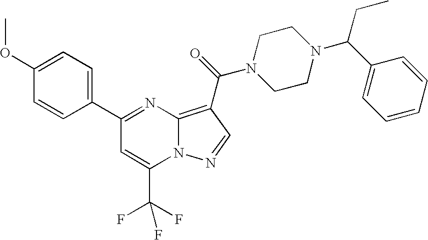 |
| Molecular Weight | 523.55 |
| Name | methanone, [5-(4-methoxyphenyl)-7- |
| | (trifluoromethyl)pyrazolo[1,5-a]pyrimidin-3-yl][4-(1- |
| | phenylpropyl)-1-piperazinyl]- |
| 6HB IC50 | ++ |
| Nl4 | ND |
| Structure | 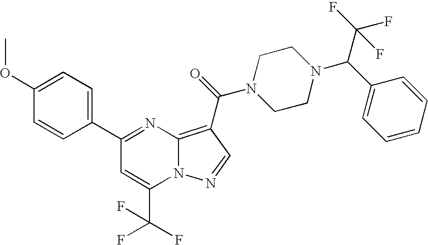 |
| Molecular Weight | 563.49 |
| Name | methanone, [5-(4-methoxyphenyl)-7- |
| | (trifluoromethyl)pyrazolo[1,5-α]pyrimidin-3-yl][4-(2,2,2- |
| | trifluoro-1-phenylethyl)-1-piperazinyl]- |
| 6HB IC50 | +++ |
| Nl4 | ND |
| Structure | 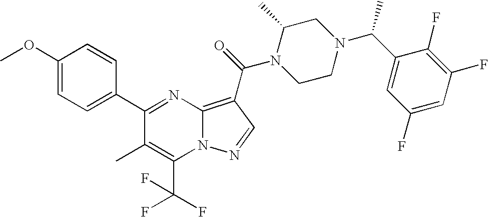 |
| Molecular Weight | 591.55 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[(1R)-1-(2,3,5-trifluorophenyl)ethyl]-1-piperazinyl]- |
| 6HB IC50 | +++ |
| Nl4 | +++ |
| Structure | 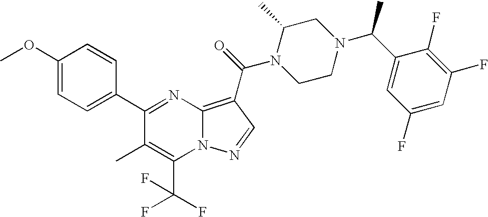 |
| Molecular Weight | 591.55 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[(1S)-1-(2,3,5-trifluorophenyl)ethyl]-1-piperazinyl]- |
| 6HB IC50 | +++ |
| Nl4 | +++ |
| Structure | 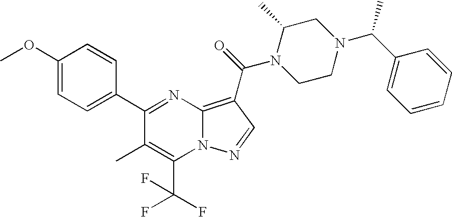 |
| Molecular Weight | 537.58 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[(1R)-1-phenylethyl]-1-piperazinyl]- |
| 6HB IC50 | +++ |
| Nl4 | +++ |
| Structure | 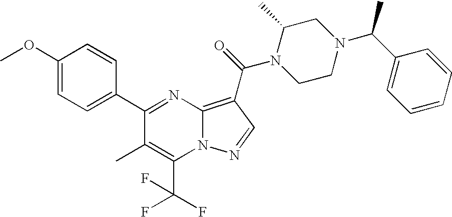 |
| Molecular Weight | 537.58 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[(1S)-1-phenylethyl]-1-piperazinyl]- |
| 6HB IC50 | +++ |
| Nl4 | +++ |
| Structure | 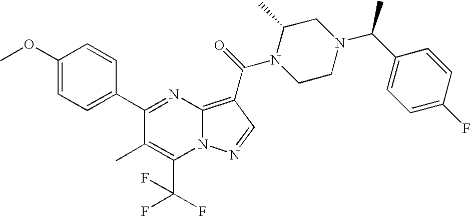 |
| Molecular Weight | 555.57 |
| Name | methanone, [(2R)-4-[(1S)-1-(4-fluorophenyl)ethyl]-2-methyl-1- |
| | piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 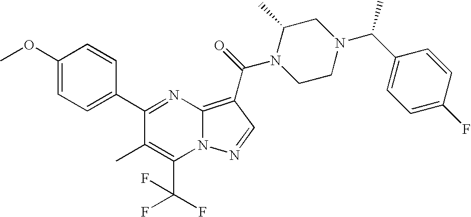 |
| Molecular Weight | 555.57 |
| Name | methanone, [(2R)-4-[(1R)-1-(4-fluorophenyl)ethyl]-2-methyl-1- |
| | piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 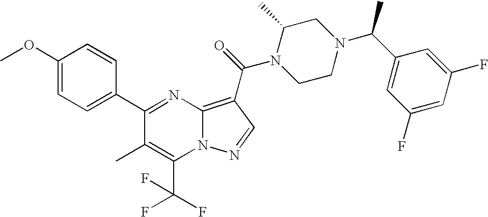 |
| Molecular Weight | 573.56 |
| Name | methanone, [(2R)-4-[(1S)-1-(3,5-difluorophenyl)ethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 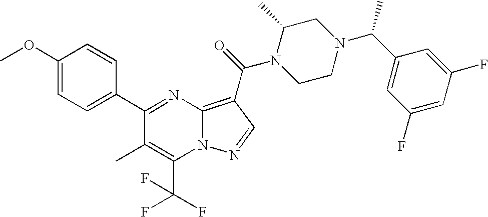 |
| Molecular Weight | 573.56 |
| Name | methanone, [(2R)-4-[(1R)-1-(3,5-difluorophenyl)ethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 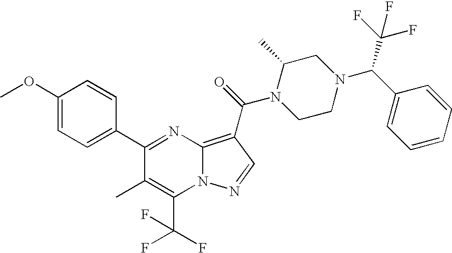 |
| Molecular Weight | 591.55 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[(1S)-2,2,2-trifluoro-1-phenylethyl]-1-piperazinyl]- |
| 6HB IC50 | +++ |
| Nl4 | +++ |
| Structure | 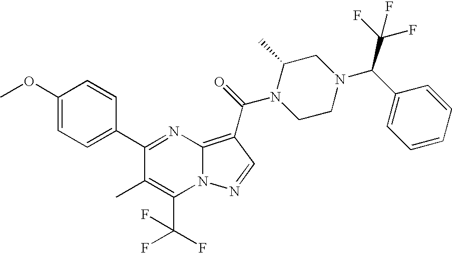 |
| Molecular Weight | 591.55 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[(1R)-2,2,2-trifluoro-1-phenylethyl]-1-piperazinyl]- |
| 6HB IC50 | +++ |
| Nl4 | +++ |
| Structure | 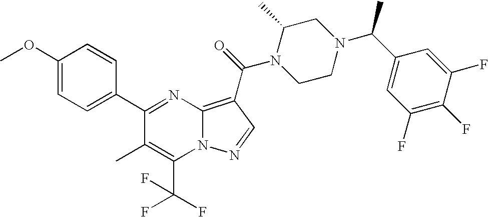 |
| Molecular Weight | 591.55 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[(1S)-1-(3,4,5-trifluorophenyl)ethyl]-1-piperazinyl]- |
| 6HB IC50 | +++ |
| Nl4 | +++ |
| Structure | 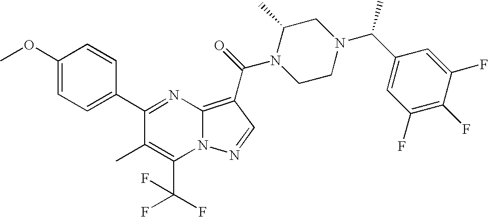 |
| Molecular Weight | 591.55 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[(1R)-1-(3,4,5-trifluorophenyl)ethyl]-1-piperazinyl]- |
| 6HB IC50 | +++ |
| Nl4 | +++ |
| Structure | 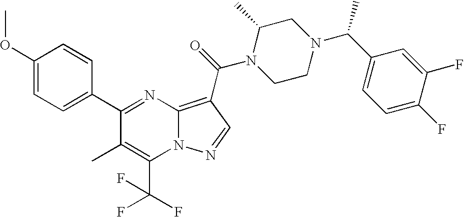 |
| Molecular Weight | 573.56 |
| Name | methanone, [(2R)-4-[(1R)-1-(3,4-difluorophenyl)ethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 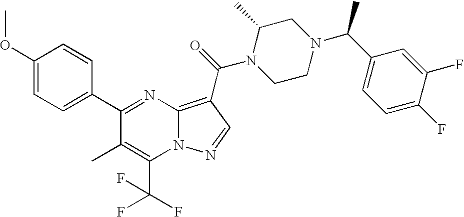 |
| Molecular Weight | 573.56 |
| Name | methanone, [(2R)-4-[(1S)-1-(3,4-difluorophenyl)ethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 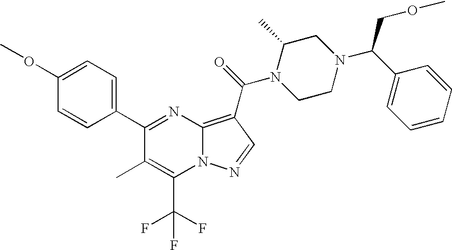 |
| Molecular Weight | 567.60 |
| Name | methanone, [(2R)-4-[(1R)-2-methoxy-1-phenylethyl]-2-methyl- |
| | 1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 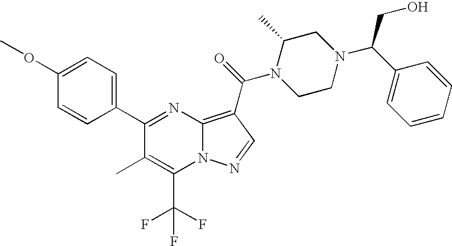 |
| Molecular Weight | 553.58 |
| Name | methanone, [(2R)-4-[(1R)-2-hydroxy-1-phenylethyl]-2-methyl- |
| | 1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 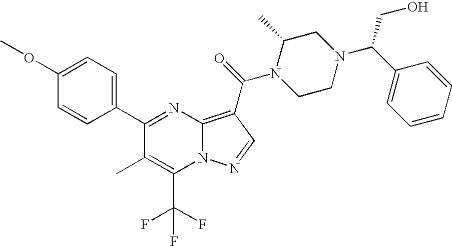 |
| Molecular Weight | 553.58 |
| Name | methanone, [(2R)-4-[(1S)-2-hydroxy-1-phenylethyl]-2-methyl- |
| | 1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 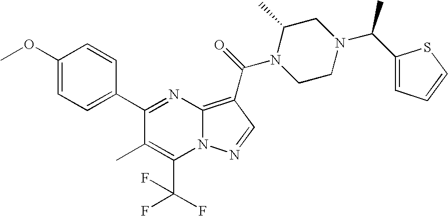 |
| Molecular Weight | 543.6 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[(1S)-1-(2-thienyl)ethyl]-1-piperazinyl]- |
| 6HB IC50 | +++ |
| Nl4 | +++ |
| Structure | 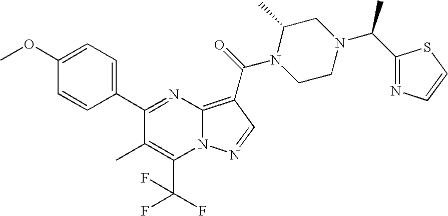 |
| Molecular Weight | 544.59 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[(1S)-1-(2-thiazolyl)ethyl]-1-piperazinyl]- |
| 6HB IC50 | +++ |
| Nl4 | +++ |
| Structure | 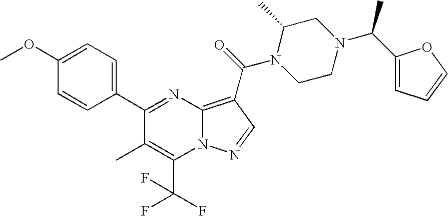 |
| Molecular Weight | 527.54 |
| Name | methanone, [(2R)-4-[(1S)-1-(2-furanyl)ethyl]-2-methyl-1- |
| | piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 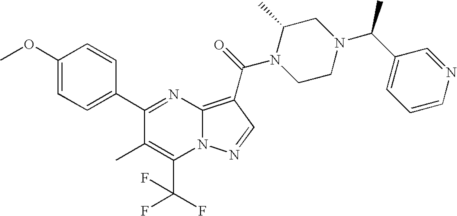 |
| Molecular Weight | 538.56 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[(1S)-1-(3-pyridinyl)ethyl]-1-piperazinyl]- |
| 6HB IC50 | +++ |
| Nl4 | +++ |
| Structure | 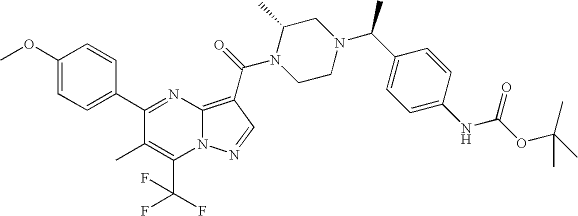 |
| Molecular Weight | 652.71 |
| Name | carbamic acid, N-[4-[(1S)-1-[(3R)-4-[[5-(4-methoxyphenyl)-6- |
| | methyl-7-(trifluoromethyl)pyrazolo[1,5-α]pyrimidin-3- |
| | yl]carbonyl]-3-methyl-1-piperazinyl]ethyl]phenyl]-, 1,1- |
| | dimethylethyl ester |
| 6HB IC50 | + |
| Nl4 | +++ |
| Structure | 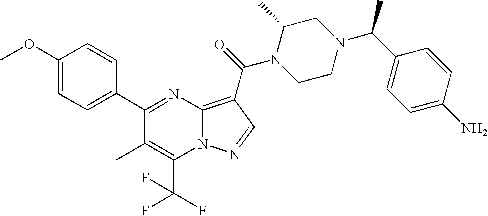 |
| Molecular Weight | 552.59 |
| Name | methanone, [(2R)-4-[(1S)-1-(4-aminophenyl)ethyl]-2-methyl-1- |
| | piperazinyl][5-(4-methoxyphenyl)-6-methyl-7- |
| | (trifluoromethyl)pyrazolo[1,5-α]pyrimidin-3-yl]- |
| 6HB IC50 | ++ |
| Nl4 | +++ |
| Structure | 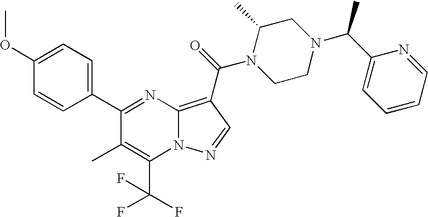 |
| Molecular Weight | 538.56 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[(1S)-1-(2-pyridinyl)ethyl]-1-piperazinyl]- |
| 6HB IC50 | +++ |
| Nl4 | +++ |
| Structure | 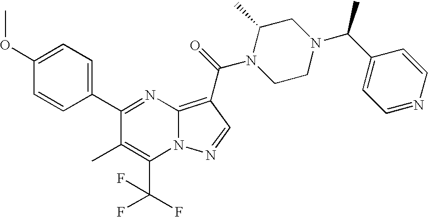 |
| Molecular Weight | 538.56 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[(1S)-1-(4-pyridinyl)ethyl]-1-piperazinyl]- |
| 6HB IC50 | +++ |
| Nl4 | +++ |
| Structure | 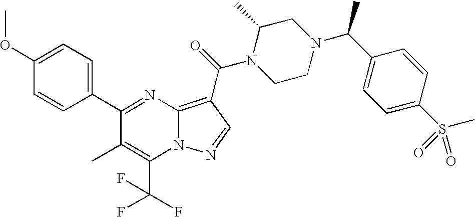 |
| Molecular Weight | 615.67 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[(1S)-1-[4-(methylsulfonyl)phenyl]ethyl]-1-piperazinyl]- |
| 6HB IC50 | ++ |
| Nl4 | ++ |
| Structure | 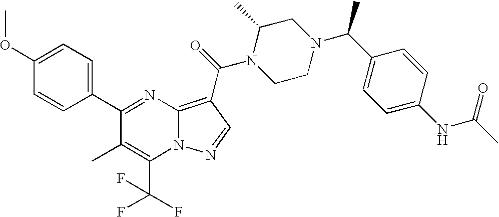 |
| Molecular Weight | 594.63 |
| Name | acetamide, N-[4-[(1S)-1-[(3R)-4-[[5-(4-methoxyphenyl)-6- |
| | methyl-7-(trifluoromethyl)pyrazolo[1,5-α]pyrimidin-3- |
| | yl]carbonyl]-3-methyl-1-piperazinyl]ethyl]phenyl]- |
| 6HB IC50 | + |
| Nl4 | ++ |
| Structure | 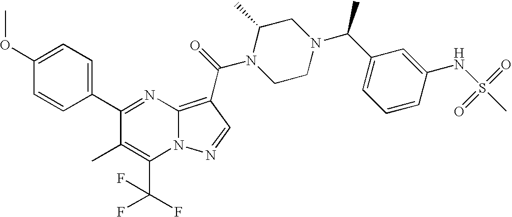 |
| Molecular Weight | 630.68 |
| Name | methanesulfonamide, N-[3-[(1S)-1-[(3R)-4-[[5-(4- |
| | methoxyphenyl)-6-methyl-7-(trifluoromethyl)pyrazolo[1,5- |
| | α]pyrimidin-3-yl]carbonyl]-3-methyl-1- |
| | piperazinyl]ethyl]phenyl]- |
| 6HB IC50 | +++ |
| Nl4 | +++ |
| Structure | 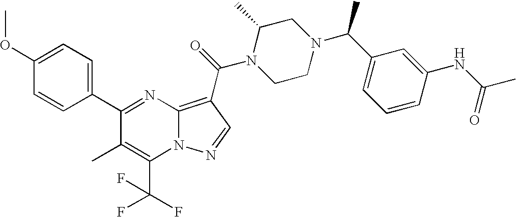 |
| Molecular Weight | 594.63 |
| Name | acetamide, N-[3-[(1S)-1-[(3R)-4-[[5-(4-methoxyphenyl)-6- |
| | methyl-7-(trifluoromethyl)pyrazolo[1,5-α]pyrimidin-3- |
| | yl]carbonyl]-3-methyl-1-piperazinyl]ethyl]phenyl]- |
| 6HB IC50 | +++ |
| Nl4 | +++ |
| Structure | 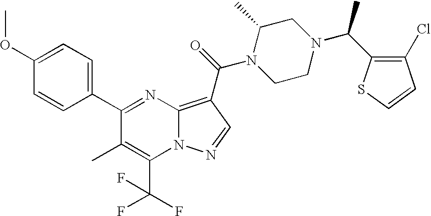 |
| Molecular Weight | 578.05 |
| Name | methanone, [(2R)-4-[(1S)-1-(3-chloro-2-thienyl)ethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 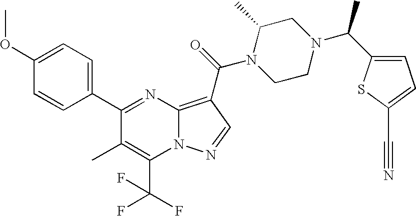 |
| Molecular Weight | 568.61 |
| Name | 2-thiophenecarbonitrile, 5-[(1S)-1-[(3R)-4-[[5-(4- |
| | methoxyphenyl)-6-methyl-7-(trifluoromethyl)pyrazolo[1,5- |
| | α]pyrimidin-3-yl]carbonyl]-3-methyl-1-piperazinyl]ethyl]- |
| 6HB IC50 | +++ |
| Nl4 | ++ |
| Structure | 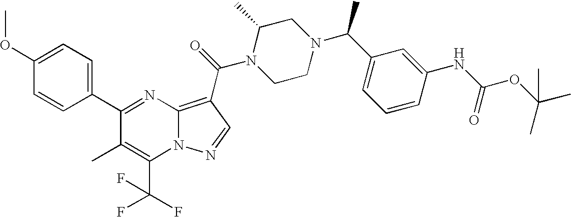 |
| Molecular Weight | 652.71 |
| Name | carbamic acid, N-[3-[(1S)-1-[(3R)-4-[[5-(4-methoxyphenyl)-6- |
| | methyl-7-(trifluoromethyl)pyrazolo[1,5-α]pyrimidin-3- |
| | yl]carbonyl]-3-methyl-1-piperazinyl]ethyl]phenyl]-, 1,1- |
| | dimethylethyl ester |
| 6HB IC50 | ++ |
| Nl4 | + |
| Structure | 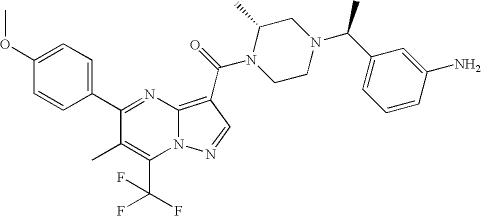 |
| Molecular Weight | 552.59 |
| Name | methanone, [(2R)-4-[(1S)-1-(3-aminophenyl)ethyl]-2-methyl-1- |
| | piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 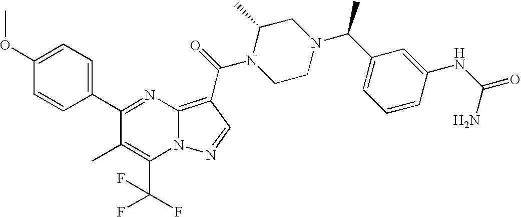 |
| Molecular Weight | 595.62 |
| Name | urea, N-[3-[(1S)-1-[(3R)-4-[[5-(4-methoxyphenyl)-6-methyl-7- |
| | (trifluoromethyl)pyrazolo[1,5-α]pyrimidin-3-yl]carbonyl]-3- |
| | methyl-1-piperazinyl]ethyl]phenyl]- |
| 6HB IC50 | +++ |
| Nl4 | +++ |
| Structure | 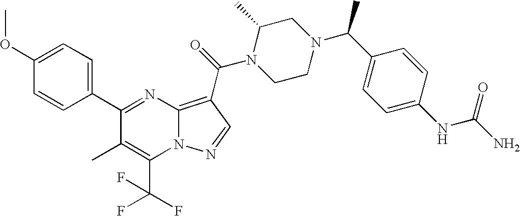 |
| Molecular Weight | 595.62 |
| Name | urea, N-[4-[(1)-1-[(3R)-4-[[5-(4-methoxyphenyl)-6-methyl-7- |
| | (trifluoromethyl)pyrazolo[1,5-α]pyrimidin-3-yl]carbonyl]-3- |
| | methyl-1-piperazinyl]ethyl]phenyl]- |
| 6HB IC50 | ++ |
| Nl4 | +++ |
| Structure | 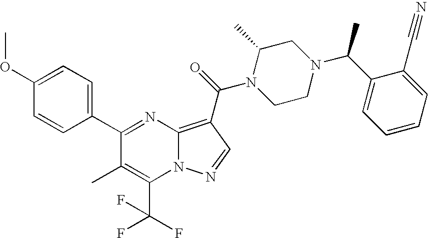 |
| Molecular Weight | 562.59 |
| Name | benzonitrile, 2-[(1S)-1-[(3R)-4-[[5-(4-methoxyphenyl)-6- |
| | methyl-7-(trifluoromethyl)pyrazolo[1,5-α]pyrimidin-3- |
| | yl]carbonyl]-3-methyl-1-piperazinyl]ethyl]- |
| 6HB IC50 | +++ |
| Nl4 | +++ |
| Structure | 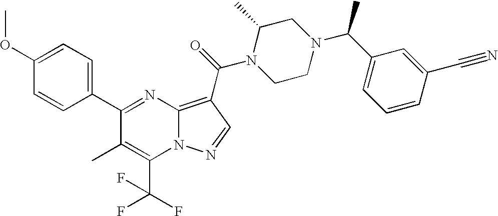 |
| Molecular Weight | 562.59 |
| Name | benzonitrile, 3-[(1S)-1-[(3R)-4-[5-(4-methoxyphenyl)-6- |
| | methyl-7-(trifluoromethyl)pyrazolo[1,5-α]pyrimidin-3- |
| | yl]carbonyl]-3-methyl-1-piperazinyl]ethyl]- |
| 6HB IC50 | +++ |
| Nl4 | +++ |
| Structure | 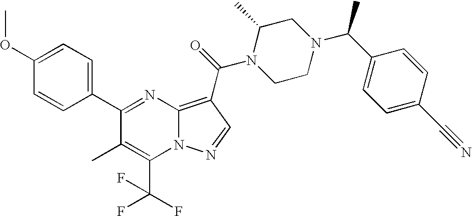 |
| Molecular Weight | 562.59 |
| Name | benzonitrile, 4-[(1S)-1-[(3R)-4-[[5-(4-methoxyphenyl)-6- |
| | methyl-7-(trifluoromethyl)pyrazolo[1,5-α]pyrimidin-3- |
| | yl]carbonyl]-3-methyl-1-piperazinyl]ethyl]- |
| 6HB IC50 | ++ |
| Nl4 | +++ |
| Structure | 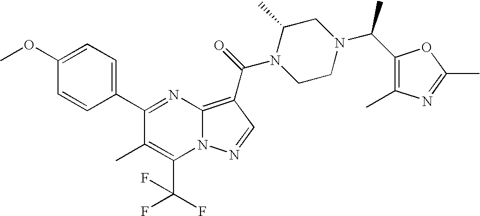 |
| Molecular Weight | 556.58 |
| Name | methanone, [(2R)-4-[(1S)-1-(2,4-dimethyl-5-oxazolyl)ethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | ++ |
| Nl4 | ++ |
| Structure | 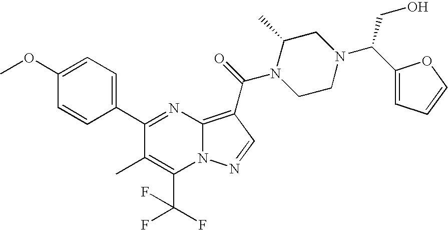 |
| Molecular Weight | 543.54 |
| Name | methanone, [(2R)-4-[(1R)-1-(2-furanyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 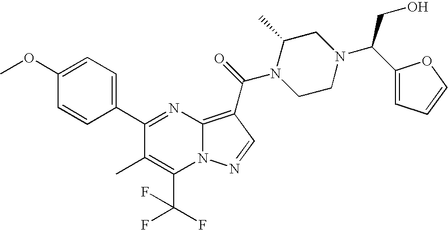 |
| Molecular Weight | 543.54 |
| Name | methanone, [(2R)-4-[(1S)-1-(2-furanyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 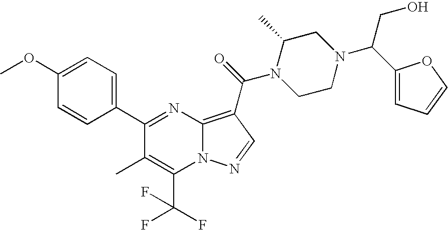 |
| Molecular Weight | 543.54 |
| Name | methanone, [(2R)-4-[1-(2-furanyl)-2-hydroxyethyl]-2-methyl-1- |
| | piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 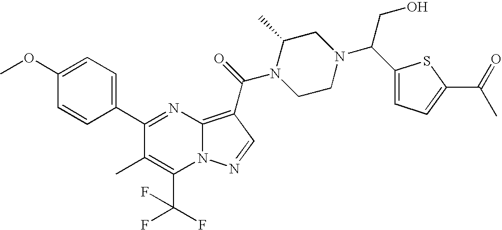 |
| Molecular Weight | 601.64 |
| Name | ethanone, 1-[5-[2-hydroxy-1-[(3R)-4-[5-(4-methoxyphenyl)-6- |
| | methyl-7-(trifluoromethyl)pyrazolo[1,5-α]pyrimidin-3- |
| | yl]carbonyl]-3-methyl-1-piperazinyl]ethyl]-2-thienyl]- |
| 6HB IC50 | ++ |
| Nl4 | +++ |
| Structure | 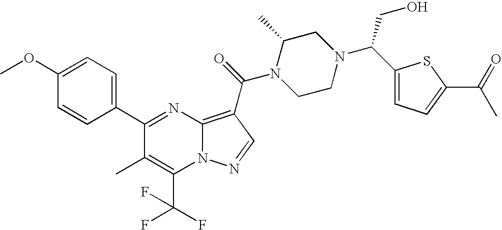 |
| Molecular Weight | 601.64 |
| Name | ethanone, 1-[5-[(1R)-2-hydroxy-1-[(3R)-4-[[5-(4- |
| | methoxyphenyl)-6-methyl-7-(trifluoromethyl)pyrazolo[1,5- |
| | α]pyrimidin-3-yl]carbonyl]-3-methyl-1-piperazinyl]ethyl]-2- |
| | thienyl]- |
| 6HB IC50 | +++ |
| Nl4 | ++ |
| Structure | 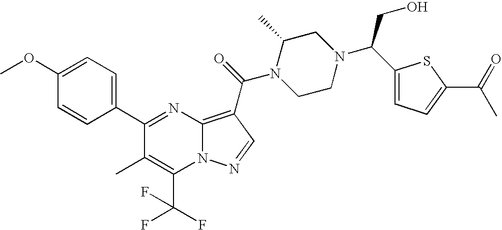 |
| Molecular Weight | 601.64 |
| Name | ethanone, 1-[5-[(1S)-2-hydroxy-1-[(3R)-4-[[5-(4- |
| | methoxyphenyl)-6-methyl-7-(trifluoromethyl)pyrazolo[1,5- |
| | α]pyrimidin-3-yl]carbonyl]-3-methyl-1-piperazinyl]ethyl]-2- |
| | thienyl]- |
| 6HB IC50 | +++ |
| Nl4 | ++ |
| Structure | 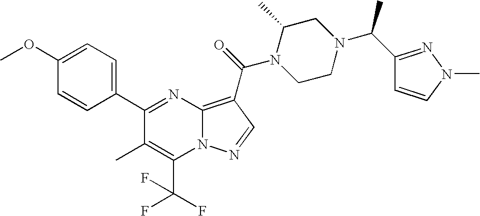 |
| Molecular Weight | 541.57 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[(1S)-1-(1-methyl-1H-pyrazol-3-yl)ethyl]-1-piperazinyl]- |
| 6HB IC50 | ++ |
| Nl4 | +++ |
| Structure | 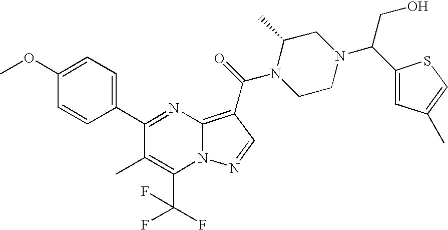 |
| Molecular Weight | 573.63 |
| Name | methanone, [(2R)-4-[2-hydroxy-1-(4-methyl-2-thienyl)ethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 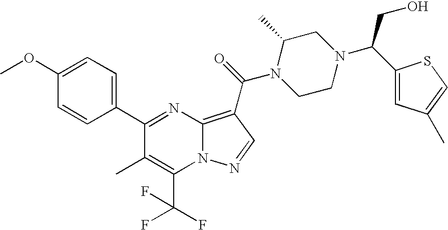 |
| Molecular Weight | 573.63 |
| Name | methanone, [(2R)-4-[(1S)-2-hydroxy-1-(4-methyl-2- |
| | thienyl)ethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 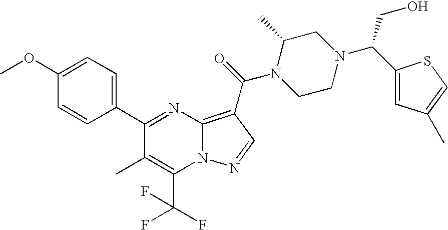 |
| Molecular Weight | 573.63 |
| Name | methanone, [(2R)-4-[(1R)-2-hydroxy-1-(4-methyl-2- |
| | thienyl)ethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 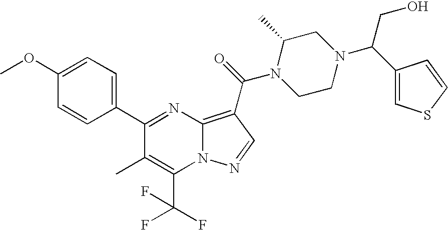 |
| Molecular Weight | 559.60 |
| Name | methanone, [(2R)-4-[2-hydroxy-1-(3-thienyl)ethyl]-2-methyl-1- |
| | piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 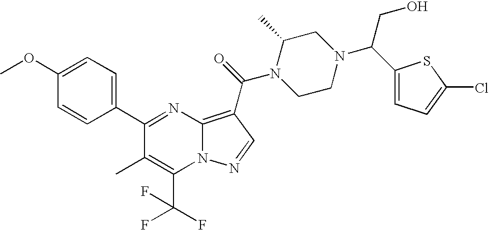 |
| Molecular Weight | 594.05 |
| Name | methanone, [(2R)-4-[1-(5-chloro-2-thienyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 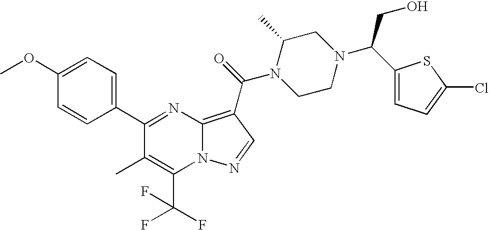 |
| Molecular Weight | 594.05 |
| Name | methanone, [(2R)-4-[(1S)-1-(5-chloro-2-thienyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 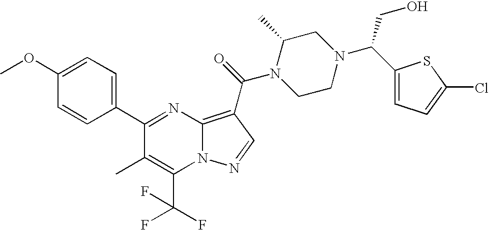 |
| Molecular Weight | 594.05 |
| Name | methanone, [(2R)-4-[(1R)-1-(5-chloro-2-thienyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 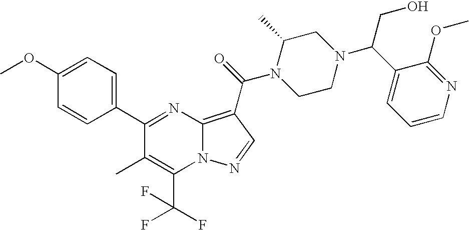 |
| Molecular Weight | 584.59 |
| Name | methanone, [(2R)-4-[2-hydroxy-1-(2-methoxy-3- |
| | pyridinyl)ethyl]-2-methyl-1-piperazinyl][5-(4-methoxyphenyl)- |
| | 6-methyl-7-(trifluoromethyl)pyrazolo[1,5-α]pyrimidin-3-yl]- |
| 6HB IC50 | +++ |
| Nl4 | +++ |
| Structure | 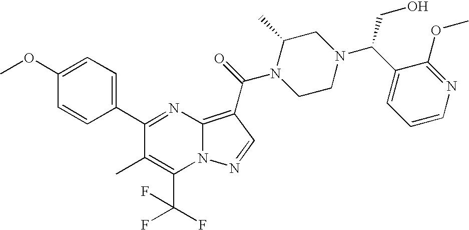 |
| Molecular Weight | 584.59 |
| Name | methanone, [(2R)-4-[(1S)-2-hydroxy-1-(2-methoxy-3- |
| | pyridinyl)ethyl]-2-methyl-1-piperazinyl][5-(4-methoxyphenyl)- |
| | 6-methyl-7-(trifluoromethyl)pyrazolo[1,5-α]pyrimidin-3-yl]- |
| 6HB IC50 | ++ |
| Nl4 | ++ |
| Structure | 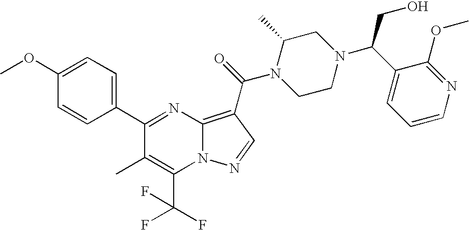 |
| Molecular Weight | 584.59 |
| Name | methanone, [(2R)-4-[(1R)-2-hydroxy-1-(2-methoxy-3- |
| | pyridinyl)ethyl]-2-methyl-1-piperazinyl][5-(4-methoxyphenyl)- |
| | 6-methyl-7-(trifluoromethyl)pyrazolo[1,5-α]pyrimidin-3-yl]- |
| 6HB IC50 | +++ |
| Nl4 | +++ |
| Structure | 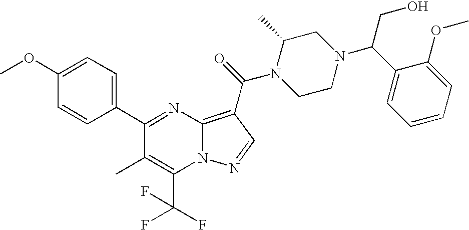 |
| Molecular Weight | 583.60 |
| Name | methanone, [(2R)-4-[2-hydroxy-1-(2 -methoxyphenyl)ethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 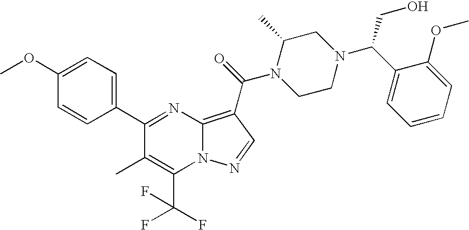 |
| Molecular Weight | 583.60 |
| Name | methanone, [(2R)-4-[(1S)-2-hydroxy-1-(2- |
| | methoxyphenyl)ethyl]-2-methyl-1-piperazinyl][5-(4- |
| | methoxyphenyl)-6-methyl-7-(trifluoromethyl)pyrazolo[1,5- |
| | α]pyrimidin-3-yl]- |
| 6HB IC50 | +++ |
| Nl4 | +++ |
| Structure | 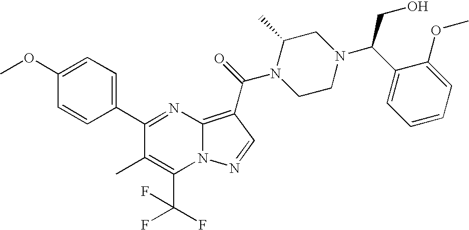 |
| Molecular Weight | 583.60 |
| Name | methanone, [(2R)-4-[(1R)-2-hydroxy-1-(2- |
| | methoxyphenyl)ethyl]-2-methyl-1-piperazinyl][5-(4- |
| | methoxyphenyl)-6-methyl-7-(trifluoromethyl)pyrazolo[1,5- |
| | α]pyrimidin-3-yl]- |
| 6HB IC50 | +++ |
| Nl4 | +++ |
| Structure | 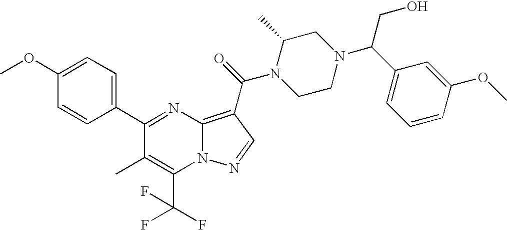 |
| Molecular Weight | 583.60 |
| Name | methanone, [(2R)-4-[2-hydroxy-1-(3-methoxyphenyl)ethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 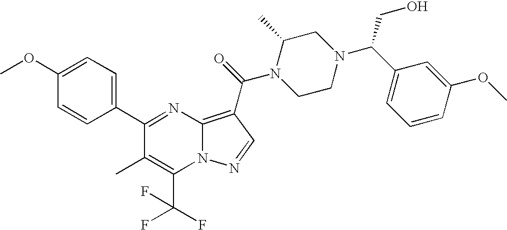 |
| Molecular Weight | 583.60 |
| Name | methanone, [(2R)-4-[(1S)-2-hydroxy-1-(3- |
| | methoxyphenyl)ethyl]-2-methyl-1-piperazinyl][5-(4- |
| | methoxyphenyl)-6-methyl-7-(trifluoromethyl)pyrazolo[1,5- |
| | α]pyrimidin-3-yl]- |
| 6HB IC50 | +++ |
| Nl4 | +++ |
| Structure | 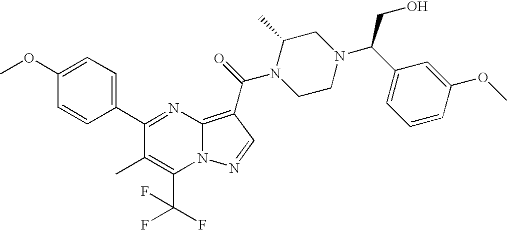 |
| Molecular Weight | 583.60 |
| Name | methanone, [(2R)-4-[(1R)-2-hydroxy-1-(3- |
| | methoxyphenyl)ethyl]-2-methyl-1-piperazinyl][5-(4- |
| | methoxyphenyl)-6-methyl-7-(trifluoromethyl)pyrazolo[1,5- |
| | α]pyrimidin-3-yl]- |
| 6HB IC50 | +++ |
| Nl4 | +++ |
| Structure | 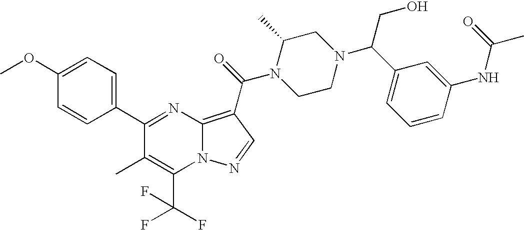 |
| Molecular Weight | 610.63 |
| Name | acetamide, N-[3-[2-hydroxy-1-[(3R)-4-[[5-(4-methoxyphenyl)- |
| | 6-methyl-7-(trifluoromethyl)pyrazolo[1,5-α]pyrimidin-3- |
| | yl]carbonyl]-3-methyl-1-piperazinyl]ethyl]phenyl]- |
| 6HB IC50 | +++ |
| Nl4 | +++ |
| Structure | 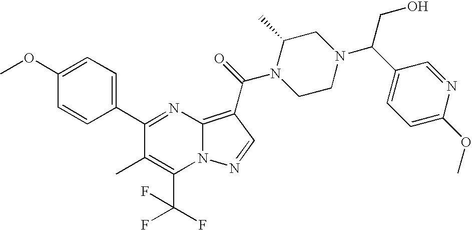 |
| Molecular Weight | 584.59 |
| Name | methanone, [(2R)-4-[2-hydroxy-1-(6-methoxy-3- |
| | pyridinyl)ethyl]-2-methyl-1-piperazinyl][5-(4-methoxyphenyl)- |
| | 6-methyl-7-(trifluoromethyl)pyrazolo[1,5-α]pyrimidin-3-yl]- |
| 6HB IC50 | +++ |
| Nl4 | +++ |
| Structure | 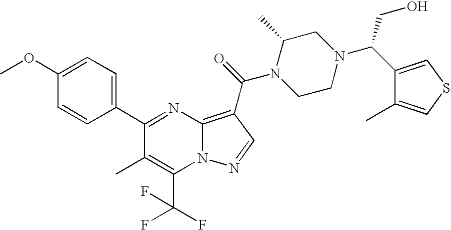 |
| Molecular Weight | 573.63 |
| Name | methanone, [(2R)-4-[(1S)-2-hydroxy-1-(4-methyl-3- |
| | thienyl)ethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 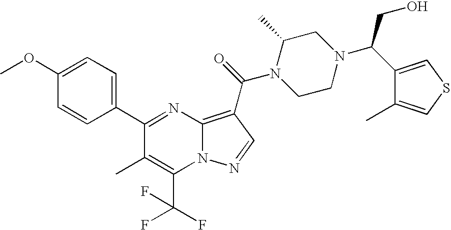 |
| Molecular Weight | 573.63 |
| Name | methanone, [(2R)-4-[(1R)-2-hydroxy-1-(4-methyl-3- |
| | thienyl)ethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 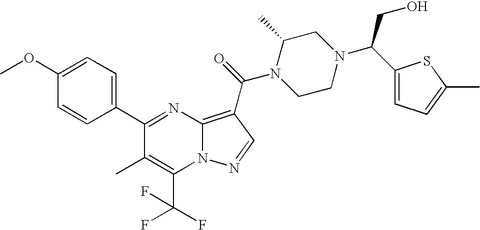 |
| Molecular Weight | 573.63 |
| Name | methanone, [(2R)-4-[(1S)-2-hydroxy-1-(5-methyl-2- |
| | thienyl)ethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 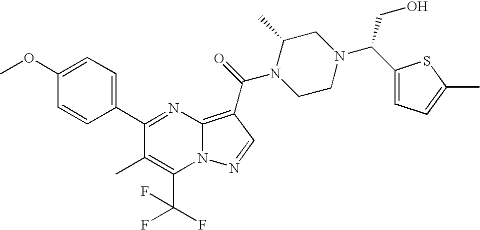 |
| Molecular Weight | 573.63 |
| Name | methanone, [(2R)-4-[(1R)-2-hydroxy-1-(5-methyl-2- |
| | thienyl)ethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 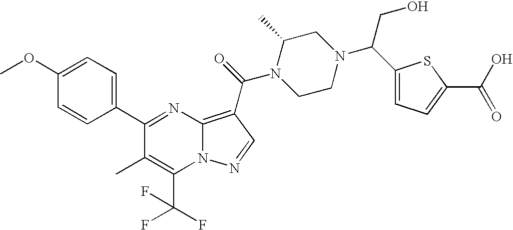 |
| Molecular Weight | 603.61 |
| Name | 2-thiophenecarboxylic acid, 5-[2-hydroxy-1-[(3R)-4-[[5-(4- |
| | methoxyphenyl)-6-methyl-7-(trifluoromethyl)pyrazolo[1,5- |
| | α]pyrimidin-3-yl]carbonyl]-3-methyl-1-piperazinyl]ethyl]- |
| 6HB IC50 | ++ |
| Nl4 | + |
| Structure | 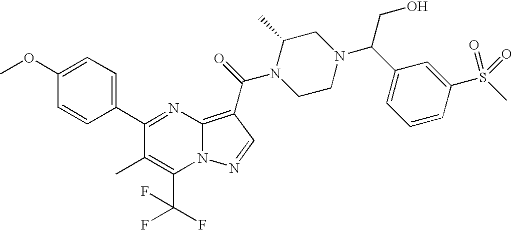 |
| Molecular Weight | 631.67 |
| Name | methanone, [(2R)-4-[2-hydroxy-1-[3- |
| | (methylsulfonyl)phenyl]ethyl]-2-methyl-1-piperazinyl][5-(4- |
| | methoxyphenyl)-6-methyl-7-(trifluoromethyl)pyrazolo[1,5- |
| | α]pyrimidin-3-yl]- |
| 6HB IC50 | + |
| Nl4 | + |
| Structure | 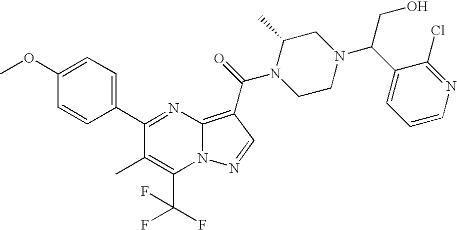 |
| Molecular Weight | 589.01 |
| Name | methanone, [(2R)-4-[1-(2-chloro-3-pyridinyl)-2-hydroxyethyl]- |
| | 2-methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 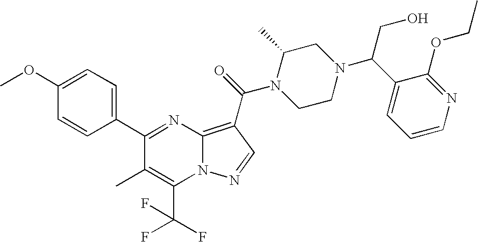 |
| Molecular Weight | 598.62 |
| Name | methanone, [(2R)-4-[1-(2-ethoxy-3-pyridinyl)-2-hydroxyethyl]- |
| | 2-methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 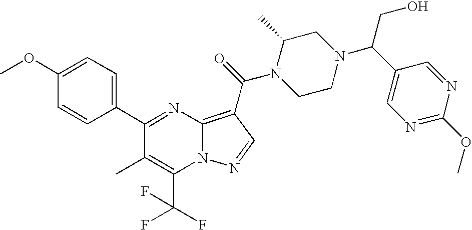 |
| Molecular Weight | 585.58 |
| Name | methanone, [(2R)-4-[2-hydroxy-1-(2-methoxy-5- |
| | pyrimidinyl)ethyl]-2-methyl-1-piperazinyl][5-(4- |
| | methoxyphenyl)-6-methyl-7-(trifluoromethyl)pyrazolo[1,5- |
| | α]pyrimidin-3-yl]- |
| 6HB IC50 | +++ |
| Nl4 | +++ |
| Structure | 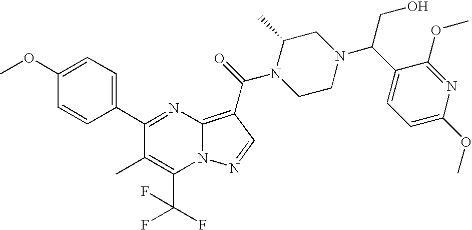 |
| Molecular Weight | 614.62 |
| Name | methanone, [(2R)-4-[1-(2,6-dimethoxy-3-pyridinyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 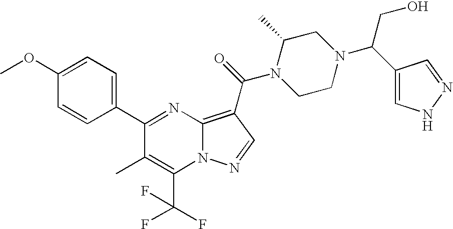 |
| Molecular Weight | 543.54 |
| Name | methanone, [(2R)-4-[2-hydroxy-1-(1H-pyrazol-4-yl)ethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | ++ |
| Nl4 | ++ |
| Structure | 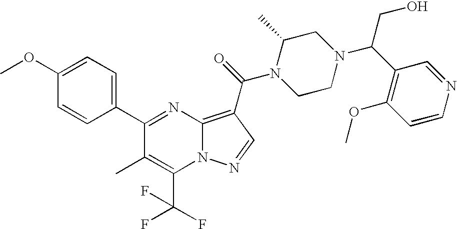 |
| Molecular Weight | 584.59 |
| Name | methanone, [(2R)-4-[2-hydroxy-1-(4-methoxy-3- |
| | pyridinyl)ethyl]-2-methyl-1-piperazinyl][5-(4-methoxyphenyl)- |
| | 6-methyl-7-(trifluoromethyl)pyrazolo[1,5-α]pyrimidin-3-yl]- |
| 6HB IC50 | +++ |
| Nl4 | +++ |
| Structure | 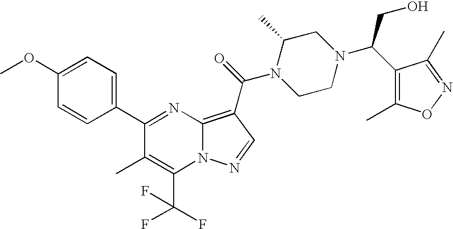 |
| Molecular Weight | 572.58 |
| Name | methanone, [(2R)-4-[(1R)-1-(3,5-dimethyl-4-isoxazolyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | ++ |
| Nl4 | ++ |
| Structure | 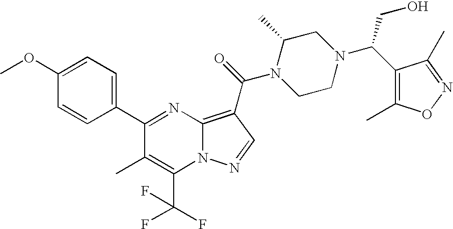 |
| Molecular Weight | 572.58 |
| Name | methanone, [(2R)-4-[(1S)-1-(3,5-dimethyl-4-isoxazolyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | ++ |
| Nl4 | ++ |
| Structure | 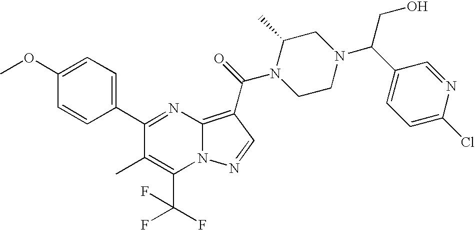 |
| Molecular Weight | 589.01 |
| Name | methanone, [(2R)-4-[1-(6-chloro-3-pyridinyl)-2-hydroxyethyl]- |
| | 2-methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 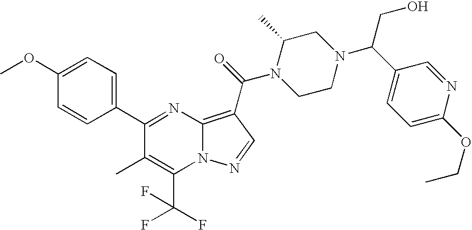 |
| Molecular Weight | 598.62 |
| Name | methanone, [(2R)-4-[1-(6-ethoxy-3-pyridinyl)-2-hydroxyethyl]- |
| | 2-methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 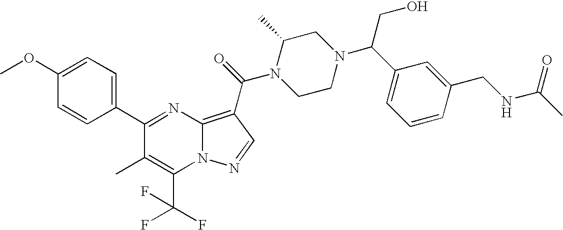 |
| Molecular Weight | 624.65 |
| Name | acetamide, N-[[3-[2-hydroxy-1-[(3R)-4-[[5-(4-methoxyphenyl)- |
| | 6-methyl-7-(trifluoromethyl)pyrazolo[1,5-α]pyrimidin-3- |
| | yl]carbonyl]-3-methyl-1-piperazinyl]ethyl]phenyl]methyl]- |
| 6HB IC50 | + |
| Nl4 | + |
| Structure | 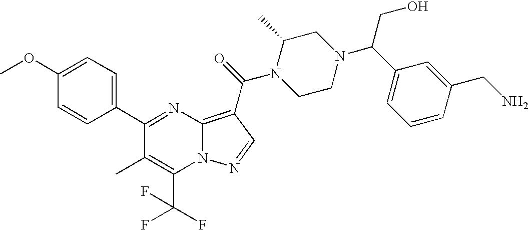 |
| Molecular Weight | 582.62 |
| Name | methanone, [(2R)-4-[1-[3-(aminomethyl)phenyl]-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | + |
| Nl4 | + |
| Structure | 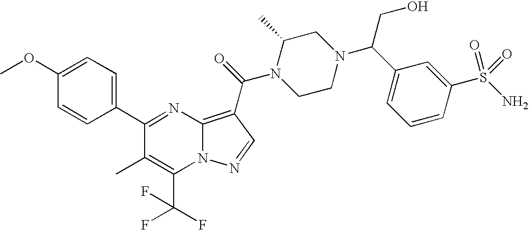 |
| Molecular Weight | 632.65 |
| Name | benzenesulfonamide, 3-[2-hydroxy-1-[(3R)-4-[[5-(4- |
| | methoxyphenyl)-6-methyl-7-(trifluoromethyl)pyrazolo[1,5- |
| | α]pyrimidin-3-yl]carbonyl]-3-methyl-1-piperazinyl]ethyl]- |
| 6HB IC50 | + |
| Nl4 | + |
| Structure | 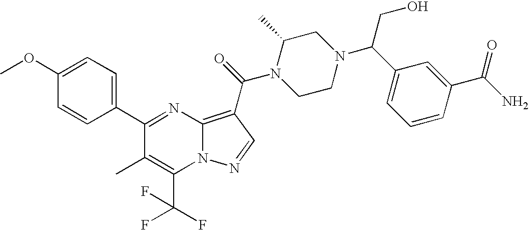 |
| Molecular Weight | 596.60 |
| Name | benzamide, 3-[2-hydroxy-1-[(3R)-4-[[5-(4-methoxyphenyl)-6- |
| | methyl-7-(trifluoromethyl)pyrazolo[1,5-α]pyrimidin-3- |
| | yl]carbonyl]-3-methyl-1-piperazinyl]ethyl]- |
| 6HB IC50 | +++ |
| Nl4 | +++ |
| Structure | 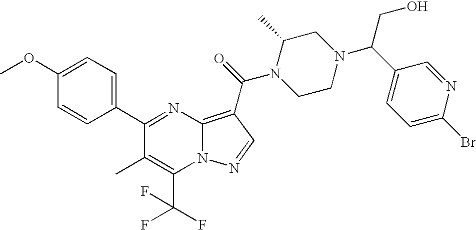 |
| Molecular Weight | 633.46 |
| Name | methanone, [(2R)-4-[1-(6-bromo-3-pyridinyl)-2-hydroxyethyl]- |
| | 2-methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 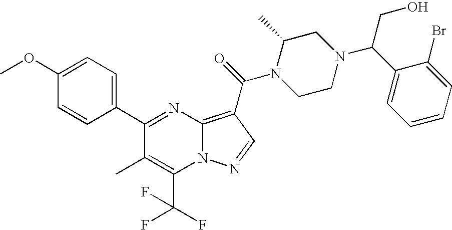 |
| Molecular Weight | 632.47 |
| Name | methanone, [(2R)-4-[1-(2-bromophenyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 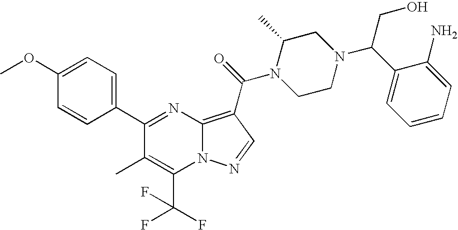 |
| Molecular Weight | 568.59 |
| Name | methanone, [(2R)-4-[1-(2-aminophenyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | ++ |
| Nl4 | ++ |
| Structure | 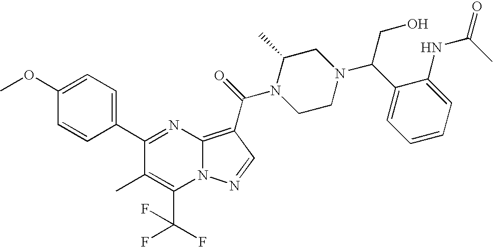 |
| Molecular Weight | 610.63 |
| Name | acetamide, N-[2-[2-hydroxy-1-[(3R)-4-[[5-(4-methoxyphenyl)- |
| | 6-methyl-7-(trifluoromethyl)pyrazolo[1,5-α]pyrimidin-3- |
| | yl]carbonyl]-3-methyl-1-piperazinyl]ethyl]phenyl]- |
| 6HB IC50 | + |
| Nl4 | + |
| Structure | 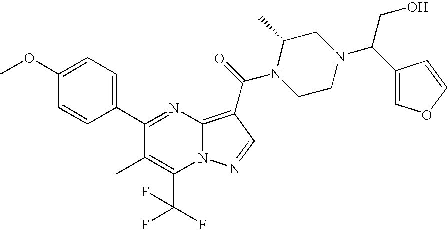 |
| Molecular Weight | 543.54 |
| Name | methanone, [(2R)-4-[1-(3-furanyl)-2-hydroxyethyl]-2-methyl-1- |
| | piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 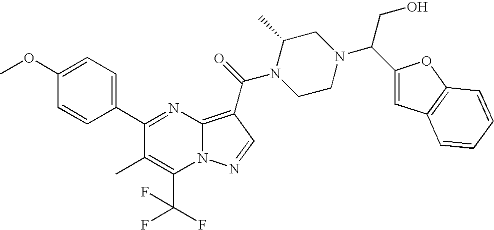 |
| Molecular Weight | 593.60 |
| Name | methanone, [(2R)-4-[1-(2-benzofuranyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 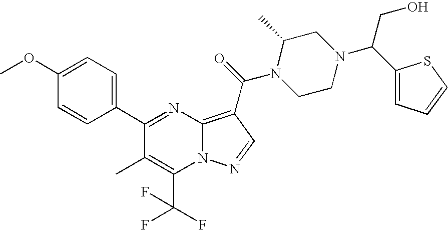 |
| Molecular Weight | 559.60 |
| Name | methanone, [(2R)-4-[2-hydroxy-1-(2-thienyl)ethyl]-2-methyl-1- |
| | piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 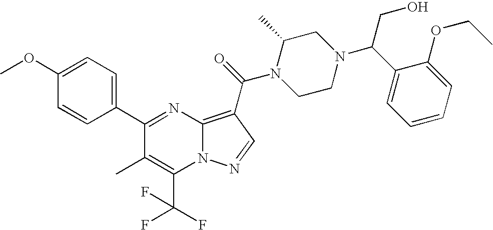 |
| Molecular Weight | 597.63 |
| Name | methanone, [(2R)-4-[1-(2-ethoxyphenyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 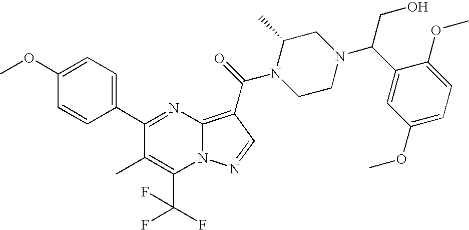 |
| Molecular Weight | 613.63 |
| Name | methanone, [(2R)-4-[1-(2,5-dimethoxyphenyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | ++ |
| Nl4 | +++ |
| Structure | 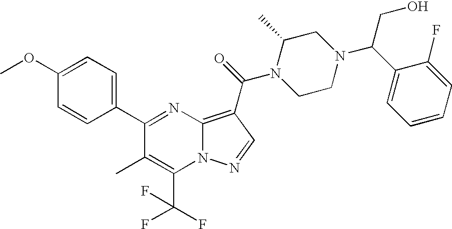 |
| Molecular Weight | 571.57 |
| Name | methanone, [(2R)-4-[1-(2-fluorophenyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 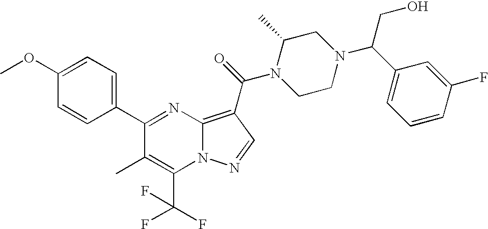 |
| Molecular Weight | 571.57 |
| Name | methanone, [(2R)-4-[1-(3-fluorophenyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 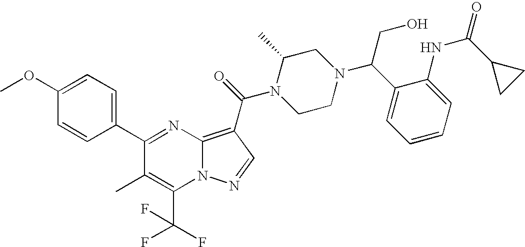 |
| Molecular Weight | 636.66 |
| Name | cyclopropanecarboxamide, N-[2-[2-hydroxy-1-[(3R)-4-[[5-(4- |
| | methoxyphenyl)-6-methyl-7-(trifluoromethyl)pyrazolo[1,5- |
| | α]pyrimidin-3-yl]carbonyl]-3-methyl-1- |
| | piperazinyl]ethyl]phenyl]- |
| 6HB IC50 | + |
| Nl4 | + |
| Structure | 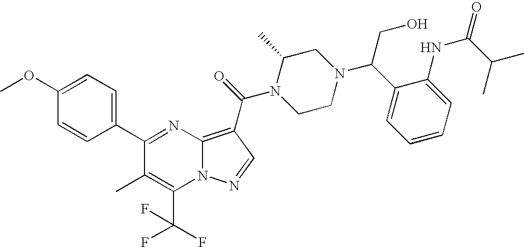 |
| Molecular Weight | 638.68 |
| Name | propanamide, N-[2-[2-hydroxy-1-[(3R)-4-[[5-(4- |
| | methoxyphenyl)-6-methyl-7-(trifluoromethyl)pyrazolo[1,5- |
| | α]pyrimidin-3-yl]carbonyl]-3-methyl-1- |
| | piperazinyl]ethyl]phenyl]-2-methyl- |
| 6HB IC50 | + |
| Nl4 | + |
| Structure | 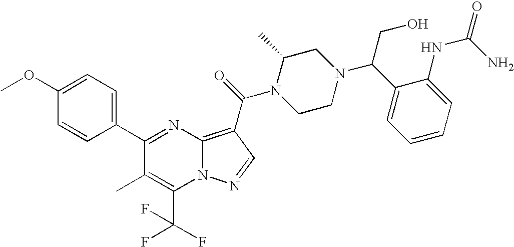 |
| Molecular Weight | 611.62 |
| Name | urea, N-[2-[2-hydroxy-1-[(3R)-4-[[5-(4-methoxyphenyl)-6- |
| | methyl-7-(trifluoromethyl)pyrazolo[1,5-α]pyrimidin-3- |
| | yl]carbonyl]-3-methyl-1-piperazinyl]ethyl]phenyl]- |
| 6HB IC50 | + |
| Nl4 | + |
| Structure | 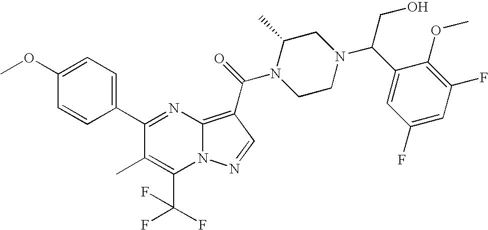 |
| Molecular Weight | 619.58 |
| Name | methanone, [(2R)-4-[1-(3,5-difluoro-2-methoxyphenyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 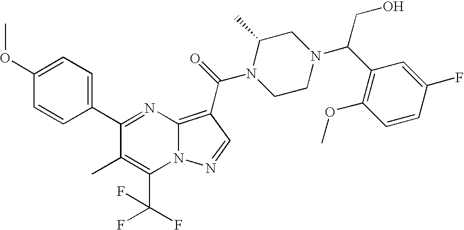 |
| Molecular Weight | 601.59 |
| Name | methanone, [(2R)-4-[1-(5-fluoro-2-methoxyphenyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 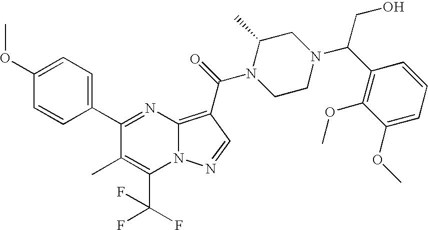 |
| Molecular Weight | 613.63 |
| Name | methanone, [(2R)-4-[1-(2,3-dimethoxyphenyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 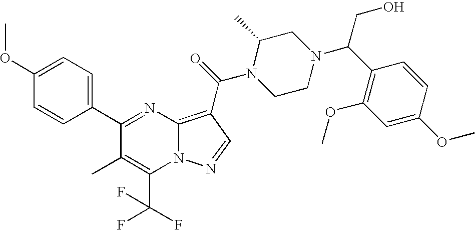 |
| Molecular Weight | 613.63 |
| Name | methanone, [(2R)-4-[1-(2,4-dimethoxyphenyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 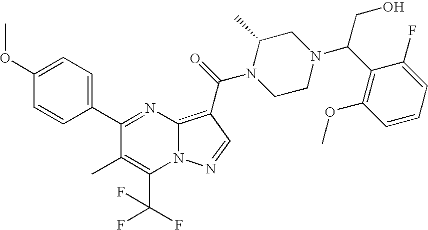 |
| Molecular Weight | 601.59 |
| Name | methanone, [(2R)-4-[1-(2-fluoro-6-methoxyphenyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 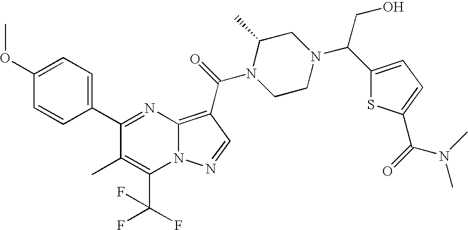 |
| Molecular Weight | 630.68 |
| Name | 2-thiophenecarboxamide, 5-[2-hydroxy-1-[(3R)-4-[[5-(4- |
| | methoxyphenyl)-6-methyl-7-(trifluoromethyl)pyrazolo[1,5- |
| | α]pyrimidin-3-yl]carbonyl]-3-methyl-1-piperazinyl]ethyl]-N,N- |
| | dimethyl- |
| 6HB IC50 | + |
| Nl4 | + |
| Structure | 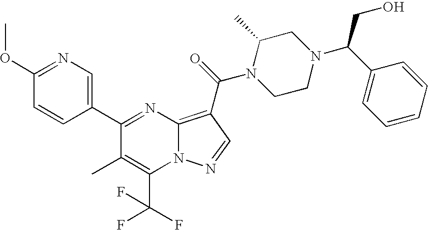 |
| Molecular Weight | 554.56 |
| Name | methanone, [(2R)-4-[(1R)-2-hydroxy-1-phenylethyl]-2-methyl- |
| | 1-piperazinyl][5-(6-methoxy-3-pyridinyl)-6-methyl-7- |
| | (trifluoromethyl)pyrazolo[1,5-α]pyrimidin-3-yl]- |
| 6HB IC50 | +++ |
| Nl4 | +++ |
| Structure | 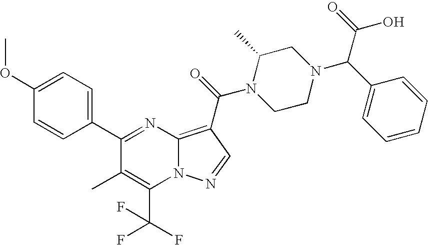 |
| Molecular Weight | 567.56 |
| Name | 1-piperazineacetic acid, 4-[[5-(4-methoxyphenyl)-6-methyl-7- |
| | (trifluoromethyl)pyrazolo[1,5-α]pyrimidin-3-yl]carbonyl]-3- |
| | methyl-α-phenyl-, (3R)- |
| 6HB IC50 | + |
| Nl4 | + |
| Structure | 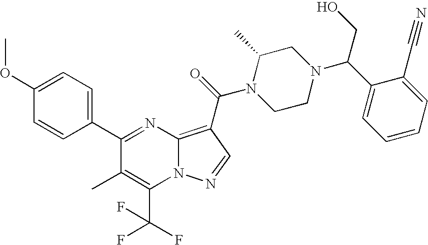 |
| Molecular Weight | 578.58 |
| Name | benzonitrile, 2-[2-hydroxy-1-[(3R)-4-[[5-(4-methoxyphenyl)-6- |
| | methyl-7-(trifluoromethyl)pyrazolo[1,5-α]pyrimidin-3- |
| | yl]carbonyl]-3-methyl-1-piperazinyl]ethyl]- |
| 6HB IC50 | +++ |
| Nl4 | +++ |
| Structure | 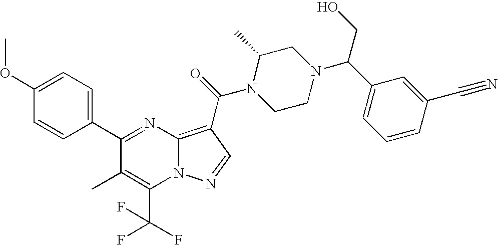 |
| Molecular Weight | 578.58 |
| Name | benzonitrile, 3-[2-hydroxy-1-[(3R)-4-[[5-(4-methoxyphenyl)-6- |
| | methyl-7-(trifluoromethyl)pyrazolo[1,5-α]pyrimidin-3- |
| | yl]carbonyl]-3-methyl-1-piperazinyl]ethyl]- |
| 6HB IC50 | +++ |
| Nl4 | +++ |
| Structure | 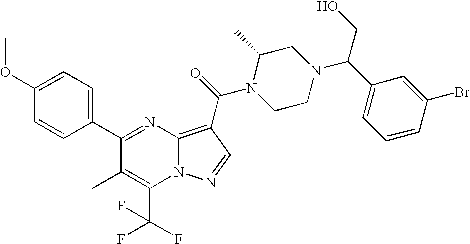 |
| Molecular Weight | 632.47 |
| Name | methanone, [(2R)-4-[1-(3-bromophenyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 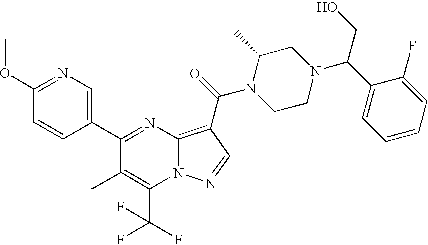 |
| Molecular Weight | 572.55 |
| Name | methanone, [(2R)-4-[1-(2-fluorophenyl)-2-hydroxyethyl]-2- |
| | methyl-1-piperazinyl][5-(6-methoxy-3-pyridinyl)-6-methyl-7- |
| | (trifluoromethyl)pyrazolo[1,5-α]pyrimidin-3-yl]- |
| 6HB IC50 | +++ |
| Nl4 | +++ |
| Structure | 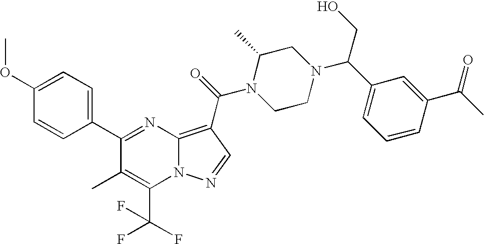 |
| Molecular Weight | 595.61 |
| Name | ethanone, 1-[3-[2-hydroxy-1-[(3R)-4-[[5-(4-methoxyphenyl)-6- |
| | methyl-7-(trifluoromethyl)pyrazolo[1,5-α]pyrimidin-3- |
| | yl]carbonyl]-3-methyl-1-piperazinyl]ethyl]phenyl]- |
| 6HB IC50 | +++ |
| Nl4 | +++ |
| Structure | 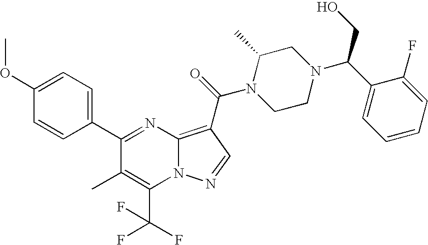 |
| Molecular Weight | 571.57 |
| Name | methanone, [(2R)-4-[(1R)-1-(2-fluorophenyl)-2-hydroxyethyl]- |
| | 2-methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 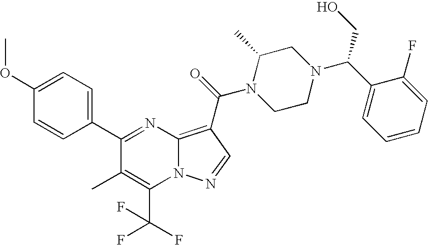 |
| Molecular Weight | 571.57 |
| Name | methanone, [(2R)-4-[(1S)-1-(2-fluorophenyl)-2-hydroxyethyl]- |
| | 2-methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 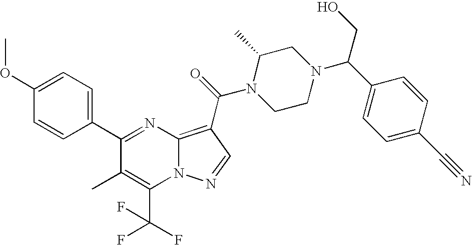 |
| Molecular Weight | 578.58 |
| Name | benzonitrile, 4-[2-hydroxy-1-[(3R)-4-[5-(4-methoxyphenyl)-6- |
| | methyl-7-(trifluoromethyl)pyrazolo[1,5-α]pyrimidin-3- |
| | yl]carbonyl]-3-methyl-1-piperazinyl]ethyl]- |
| 6HB IC50 | ++ |
| Nl4 | ++ |
| Structure | 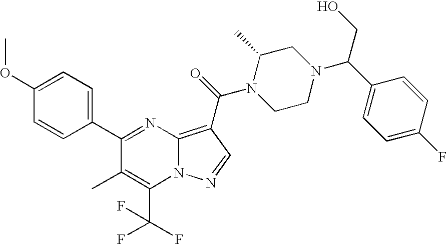 |
| Molecular Weight | 571.57 |
| Name | methanone, [(2R)-4-[1-(4-fluorophenyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 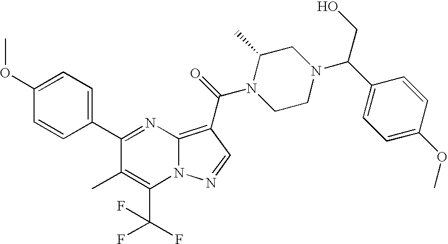 |
| Molecular Weight | 583.60 |
| Name | methanone, [(2R)-4-[2-hydroxy-1-(4-methoxyphenyl)ethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 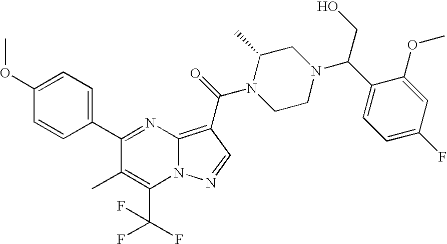 |
| Molecular Weight | 601.59 |
| Name | methanone, [(2R)-4-[1-(4-fluoro-2-methoxyphenyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 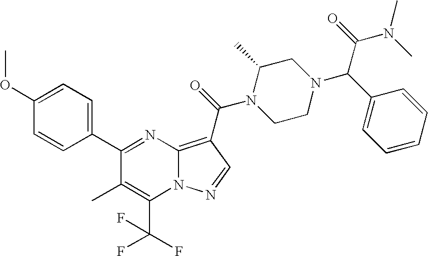 |
| Molecular Weight | 594.63 |
| Name | 1-piperazineacetamide, 4-[[5-(4-methoxyphenyl)-6-methyl-7- |
| | (trifluoromethyl)pyrazolo[1,5-α]pyrimidin-3-yl]carbonyl]- |
| | N,N,3-trimethyl-α-phenyl-, (3R)- |
| 6HB IC50 | +++ |
| Nl4 | +++ |
| Structure | 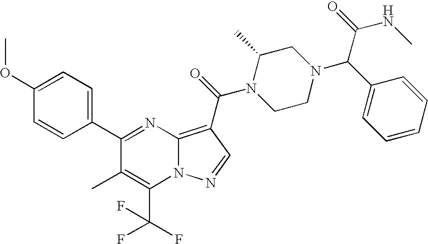 |
| Molecular Weight | 580.60 |
| Name | 1-piperazineacetamide, 4-[[5-(4-methoxyphenyl)-6-methyl-7- |
| | (trifluoromethyl)pyrazolo[1,5-α]pyrimidin-3-yl]carbonyl]-N,3- |
| | dimethyl-α-phenyl-, (3R)- |
| 6HB IC50 | +++ |
| Nl4 | +++ |
| Structure | 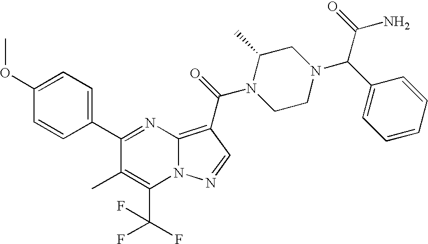 |
| Molecular Weight | 566.57 |
| Name | 1-piperazineacetamide, 4-[[5-(4-methoxyphenyl)-6-methyl-7- |
| | (trifluoromethyl)pyrazolo[1,5-α]pyrimidin-3-yl]carbonyl]-3- |
| | methyl-α-phenyl-, (3R)- |
| 6HB IC50 | +++ |
| Nl4 | +++ |
| Structure | 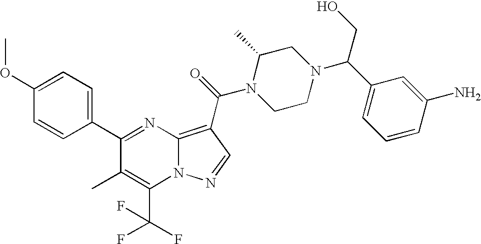 |
| Molecular Weight | 568.59 |
| Name | methanone, [(2R)-4-[1-(3-aminophenyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 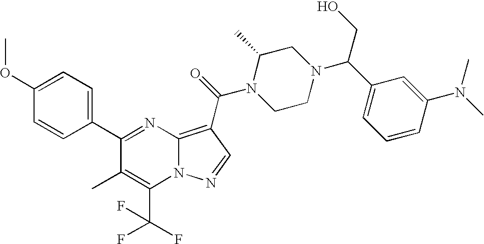 |
| Molecular Weight | 596.64 |
| Name | methanone, [(2R)-4-[1-[3-(dimethylamino)phenyl]-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 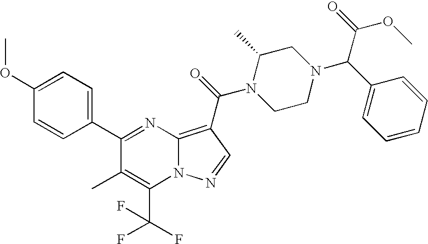 |
| Molecular Weight | 581.59 |
| Name | 1-piperazineacetic acid, 4-[[5-(4-methoxyphenyl)-6-methyl-7- |
| | (trifluoromethyl)pyrazolo[1,5-α]pyrimidin-3-yl]carbonyl]-3- |
| | methyl-α-phenyl-, methyl ester, (3R)- |
| 6HB IC50 | +++ |
| Nl4 | +++ |
| Structure | 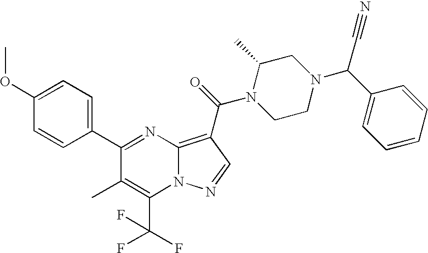 |
| Molecular Weight | 548.56 |
| Name | 1-piperazineacetonitrile, 4-[[5-(4-methoxyphenyl)-6-methyl-7- |
| | (trifluoromethyl)pyrazolo[1,5-α]pyrimidin-3-yl]carbonyl]-3- |
| | methyl-α-phenyl-, (3R)- |
| 6HB IC50 | +++ |
| Nl4 | +++ |
| Structure | 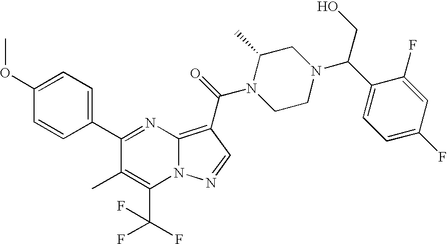 |
| Molecular Weight | 589.56 |
| Name | methanone, [(2R)-4-[1-(2,4-difluorophenyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 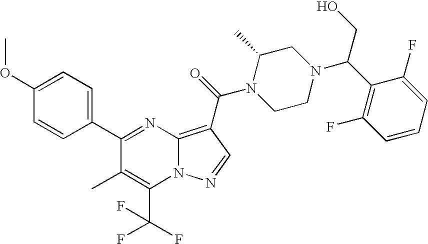 |
| Molecular Weight | 589.56 |
| Name | methanone, [(2R)-4-[1-(2,6-difluorophenyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 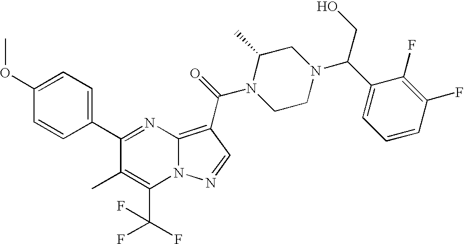 |
| Molecular Weight | 589.56 |
| Name | methanone, [(2R)-4-[1-(2,3-difluorophenyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 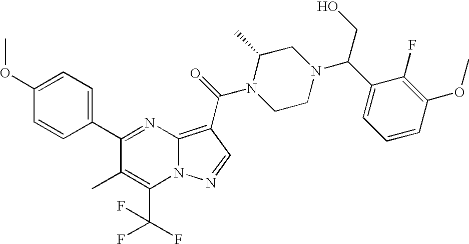 |
| Molecular Weight | 601.59 |
| Name | methanone, [(2R)-4-[1-(2-fluoro-3-methoxyphenyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 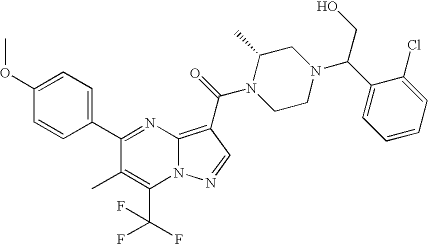 |
| Molecular Weight | 588.02 |
| Name | methanone, [(2R)-4-[1-(2-chlorophenyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 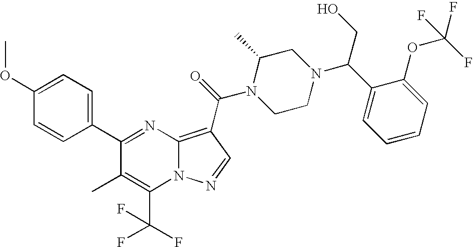 |
| Molecular Weight | 637.57 |
| Name | methanone, [(2R)-4-[2-hydroxy-1-[2- |
| | (trifluoromethoxy)phenyl]ethyl]-2-methyl-1-piperazinyl][5-(4- |
| | methoxyphenyl)-6-methyl-7-(trifluoromethyl)pyrazolo[1,5- |
| | α]pyrimidin-3-yl]- |
| 6HB IC50 | ++ |
| Nl4 | ++ |
| Structure | 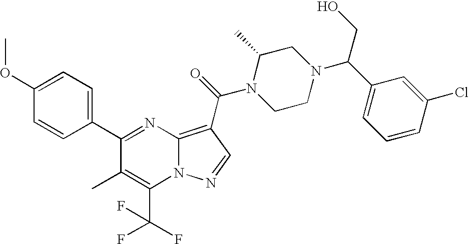 |
| Molecular Weight | 588.02 |
| Name | methanone, [(2R)-4-[1-(3-chlorophenyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 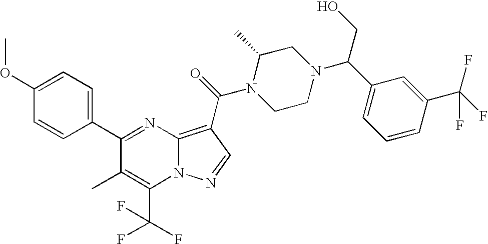 |
| Molecular Weight | 621.57 |
| Name | methanone, [(2R)-4-[2-hydroxy-1-[3- |
| | (trifluoromethyl)phenyl]ethyl]-2-methyl-1-piperazinyl][5-(4- |
| | methoxyphenyl)-6-methyl-7-(trifluoromethyl)pyrazolo[1,5- |
| | α]pyrimidin-3-yl]- |
| 6HB IC50 | +++ |
| Nl4 | ++ |
| Structure | 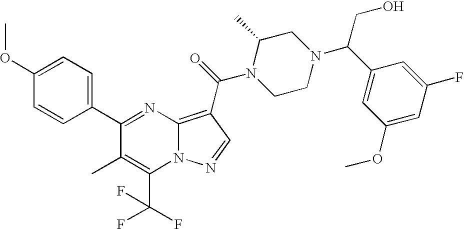 |
| Molecular Weight | 601.59 |
| Name | methanone, [(2R)-4-[1-(3-fluoro-5-methoxyphenyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 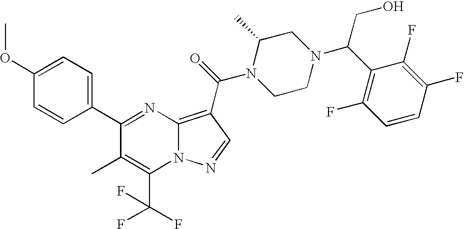 |
| Molecular Weight | 607.55 |
| Name | methanone, [(2R)-4-[2-hydroxy-1-(2,3,6-trifluorophenyl)ethyl]- |
| | 2-methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 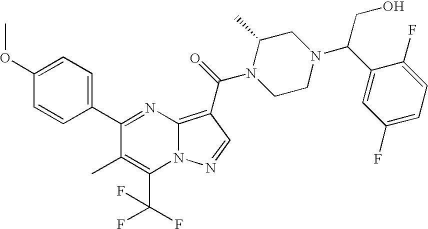 |
| Molecular Weight | 589.56 |
| Name | methanone, [(2R)-4-[1-(2,5-difluorophenyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 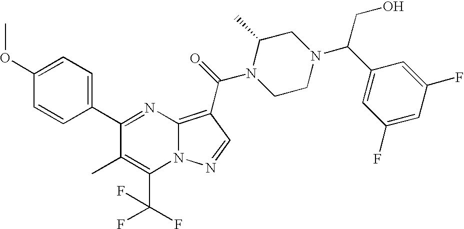 |
| Molecular Weight | 589.56 |
| Name | methanone, [(2R)-4-[1-(3,5-difluorophenyl)-2-hydroxyethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 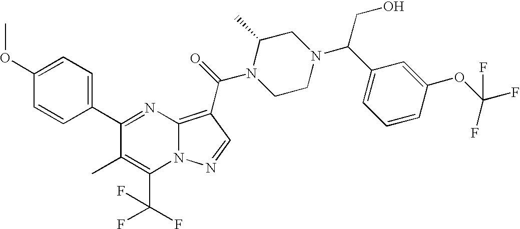 |
| Molecular Weight | 637.57 |
| Name | methanone, [(2R)-4-[2-hydroxy-1-[3- |
| | (trifluoromethoxy)phenyl]ethyl]-2-methyl-1-piperazinyl][5-(4- |
| | methoxyphenyl)-6-methyl-7-(trifluoromethyl)pyrazolo[1,5- |
| | α]pyrimidin-3-yl]- |
| 6HB IC50 | +++ |
| Nl4 | +++ |
| Structure | 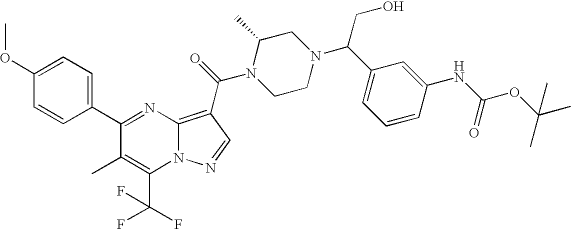 |
| Molecular Weight | 668.71 |
| Name | carbamic acid, N-[3-[2-hydroxy-1-[(3R)-4-[[5-(4- |
| | methoxyphenyl)-6-methyl-7-(trifluoromethyl)pyrazolo[1,5- |
| | α]pyrimidin-3-yl]carbonyl]-3-methyl-1- |
| | piperazinyl]ethyl]phenyl]-, 1,1-dimethylethyl ester |
| 6HB IC50 | ++ |
| Nl4 | ++ |
| Structure | 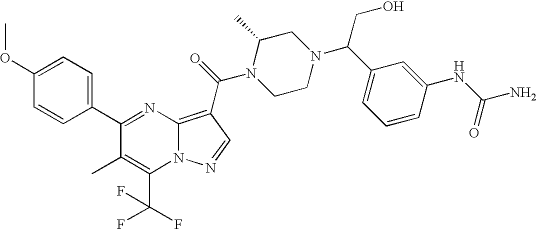 |
| Molecular Weight | 611.62 |
| Name | urea, N-[3-[2-hydroxy-1-[(3R)-4-[[5-(4-methoxyphenyl)-6- |
| | methyl-7-(trifluoromethyl)pyrazolo[1,5-α]pyrimidin-3- |
| | yl]carbonyl]-3-methyl-1-piperazinyl]ethyl]phenyl]- |
| 6HB IC50 | +++ |
| Nl4 | ++ |
| Structure | 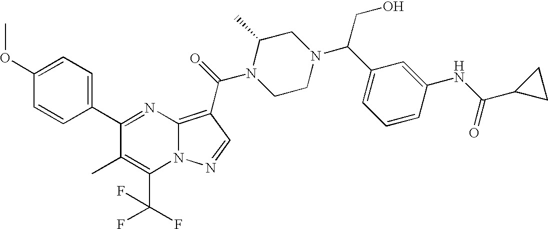 |
| Molecular Weight | 636.66 |
| Name | cyclopropanecarboxamide, N-[3-[2-hydroxy-1-[(3R)-4-[[5-(4- |
| | methoxyphenyl)-6-methyl-7-(trifluoromethyl)pyrazolo[1,5- |
| | α]pyrimidin-3-yl]carbonyl]-3-methyl-1- |
| | piperazinyl]ethyl]phenyl]- |
| 6HB IC50 | +++ |
| Nl4 | +++ |
| Structure | 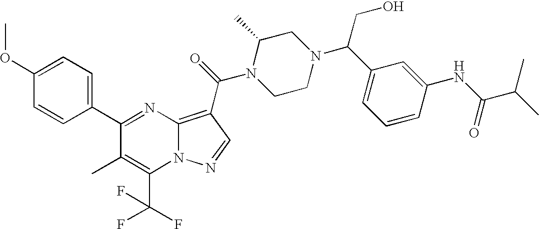 |
| Molecular Weight | 638.68 |
| Name | propanamide, N-[3-[2-hydroxy-1-[(3R)-4-[[5-(4- |
| | methoxyphenyl)-6-methyl-7-(trifluoromethyl)pyrazolo[1,5- |
| | α]pyrimidin-3-yl]carbonyl]-3-methyl-1- |
| | piperazinyl]ethyl]phenyl]-2-methyl- |
| 6HB IC50 | +++ |
| Nl4 | ++ |
| Structure | 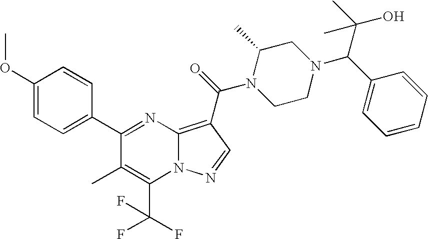 |
| Molecular Weight | 581.63 |
| Name | methanone, [(2R)-4-(2-hydroxy-2-methyl-1-phenylpropyl)-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 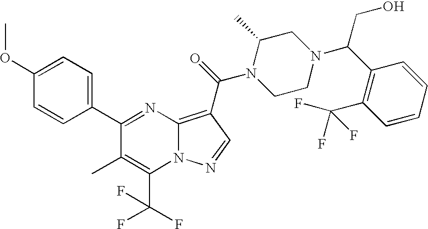 |
| Molecular Weight | 621.57 |
| Name | methanone, [(2R)-4-[2-hydroxy-1-[2- |
| | (trifluoromethyl)phenyl]ethyl]-2-methyl-1-piperazinyl][5-(4- |
| | methoxyphenyl)-6-methyl-7-(trifluoromethyl)pyrazolo[1,5- |
| | α]pyrimidin-3-yl]- |
| 6HB IC50 | +++ |
| Nl4 | +++ |
| Structure | 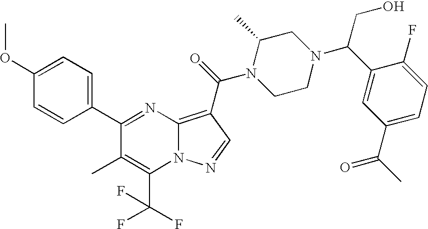 |
| Molecular Weight | 613.60 |
| Name | ethanone, 1-[4-fluoro-3-[2-hydroxy-1-[(3R)-4-[[5-(4- |
| | methoxyphenyl)-6-methyl-7-(trifluoromethyl)pyrazolo[1,5- |
| | α]pyrimidin-3-yl]carbonyl]-3-methyl-1- |
| | piperazinyl]ethyl]phenyl]- |
| 6HB IC50 | +++ |
| Nl4 | +++ |
| Structure | 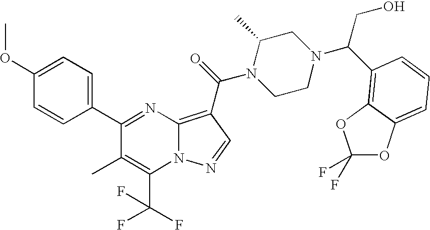 |
| Molecular Weight | 633.57 |
| Name | methanone, [(2R)-4-[1-(2,2-difluoro-1,3-benzodioxol-4-yl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 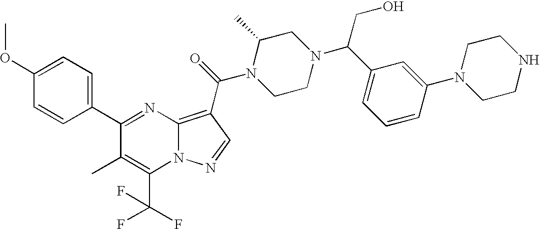 |
| Molecular Weight | 637.70 |
| Name | methanone, [(2R)-4-[2-hydroxy-1-[3-(1- |
| | piperazinyl)phenyl]ethyl]-2-methyl-1-piperazinyl][5-(4- |
| | methoxyphenyl)-6-methyl-7-(trifluoromethyl)pyrazolo[1,5- |
| | α]pyrimidin-3-yl]- |
| 6HB IC50 | + |
| Nl4 | + |
| Structure | 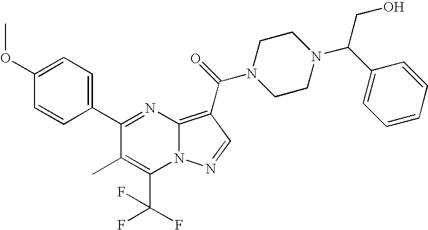 |
| Molecular Weight | 539.55 |
| Name | methanone, [4-(2-hydroxy-1-phenylethyl)-1-piperazinyl][5-(4- |
| | methoxyphenyl)-6-methyl-7-(trifluoromethyl)pyrazolo[1,5- |
| | α]pyrimidin-3-yl]- |
| 6HB IC50 | +++ |
| Nl4 | +++ |
| Structure | 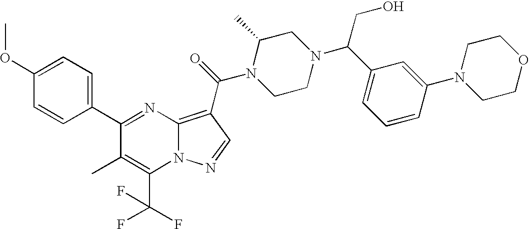 |
| Molecular Weight | 638.68 |
| Name | methanone, [(2R)-4-[2-hydroxy-1-[3-(4- |
| | morpholinyl)phenyl]ethyl]-2-methyl-1-piperazinyl][5-(4- |
| | methoxyphenyl)-6-methyl-7-(trifluoromethyl)pyrazolo[1,5- |
| | α]pyrimidin-3-yl]- |
| 6HB IC50 | ++ |
| Nl4 | ++ |
| Structure | 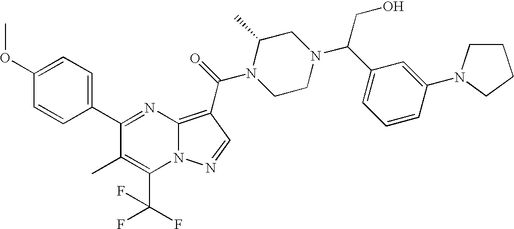 |
| Molecular Weight | 622.68 |
| Name | methanone, [(2R)-4-[2-hydroxy-1-[3-(1- |
| | pyrrolidinyl)phenyl]ethyl]-2-methyl-1-piperazinyl][5-(4- |
| | methoxyphenyl)-6-methyl-7-(trifluoromethyl)pyrazolo[1,5- |
| | α]pyrimidin-3-yl]- |
| 6HB IC50 | +++ |
| Nl4 | +++ |
| Structure | 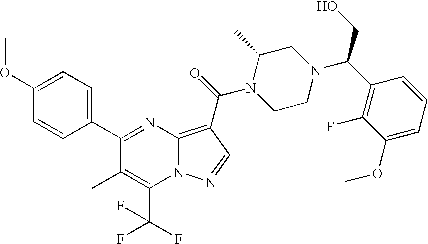 |
| Molecular Weight | 601.59 |
| Name | methanone, [(2R)-4-[(1R)-1-(2-fluoro-3-methoxyphenyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 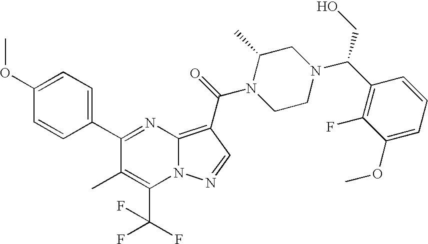 |
| Molecular Weight | 601.59 |
| Name | methanone, [(2R)-4-[(1S)-1-(2-fluoro-3-methoxyphenyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 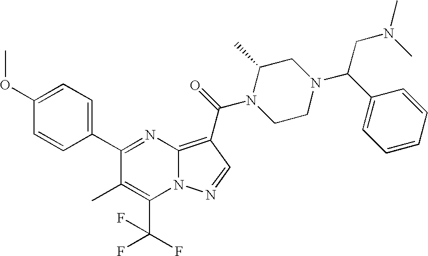 |
| Molecular Weight | 580.64 |
| Name | methanone, [(2R)-4-[2-(dimethylamino)-1-phenylethyl]-2- |
| | methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 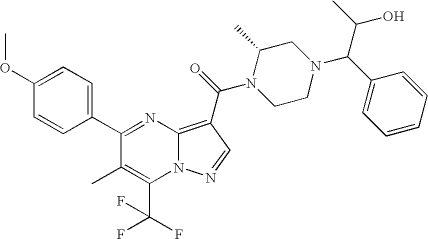 |
| Molecular Weight | 567.60 |
| Name | methanone, [(2R)-4-(2-hydroxy-1-phenylpropyl)-2-methyl-1- |
| | piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 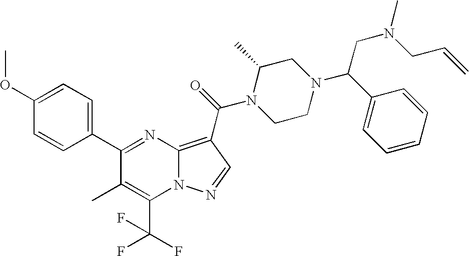 |
| Molecular Weight | 606.68 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[2-(methyl-2-propen-1-ylamino)-1-phenylethyl]-1- |
| | piperazinyl] |
| 6HB IC50 | +++ |
| Nl4 | +++ |
| Structure | 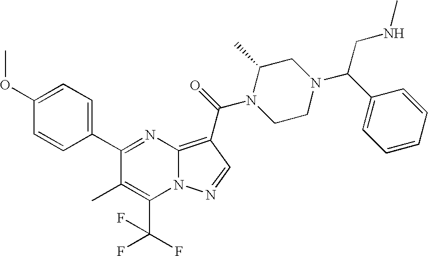 |
| Molecular Weight | 566.62 |
| Name | methanone, [5-(4-methoxyphenyl)-6-methyl-7- |
| | (trifluoromethyl)pyrazolo[1,5-α]pyrimidin-3-yl][(2R)-2-methyl- |
| | 4-[2-(methylamino)-1-phenylethyl]-1-piperazinyl]- |
| 6HB IC50 | +++ |
| Nl4 | +++ |
| Structure | 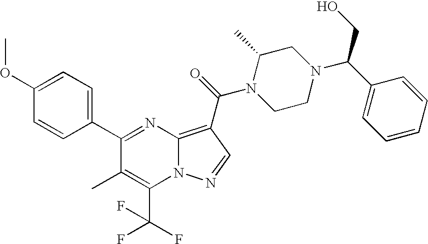 |
| Molecular Weight | 539.55 |
| Name | methanone, [(2R)-4-[(1R)-2-hydroxy-1-phenylethyl]-2-methyl- |
| | 1-piperazinyl][5-(4-methoxyphenyl)-7- |
| | (trifluoromethyl)pyrazolo[1,5-α]pyrimidin-3-yl]- |
| 6HB IC50 | +++ |
| Nl4 | +++ |
| Structure | 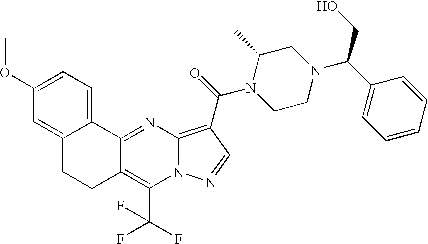 |
| Molecular Weight | 565.59 |
| Name | methanone, [5,6-dihydro-3-methoxy-7- |
| | (trifluoromethyl)benzo[h]pyrazolo[5,1-b]quinazolin-11- |
| | yl][(2R)-4-[(1R)-2-hydroxy-1-phenylethyl]-2-methyl-1- |
| | piperazinyl]- |
| 6HB IC50 | +++ |
| Nl4 | +++ |
| Structure | 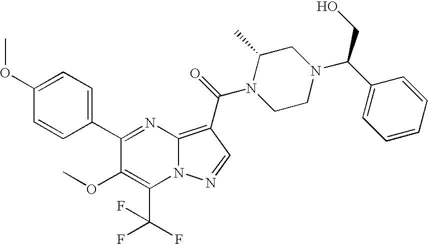 |
| Molecular Weight | 569.58 |
| Name | methanone, [(2R)-4-[(1R)-2-hydroxy-1-phenylethyl]-2-methyl- |
| | 1-piperazinyl][6-methoxy-5-(4-methoxyphenyl)-7- |
| | (trifluoromethyl)pyrazolo[1,5-α]pyrimidin-3-yl]- |
| 6HB IC50 | +++ |
| Nl4 | +++ |
| Structure | 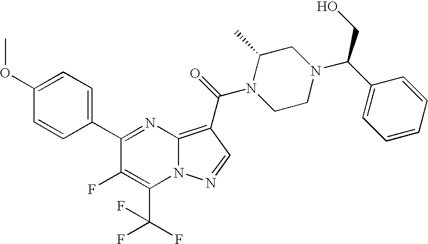 |
| Molecular Weight | 557.54 |
| Name | methanone, [6-fluoro-5-(4-methoxyphenyl)-7- |
| | (trifluoromethyl)pyrazolo[1,5-α]pyrimidin-3-yl][(2R)-4-[(1R)- |
| | 2-hydroxy-1-phenylethyl]-2-methyl-1-piperazinyl]- |
| 6HB IC50 | +++ |
| Nl4 | +++ |
| Structure | 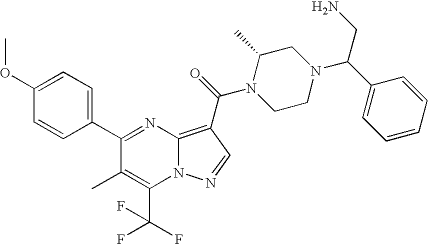 |
| Molecular Weight | 552.59 |
| Name | methanone, [(2R)-4-(2-amino-1-phenylethyl)-2-methyl-1- |
| | piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 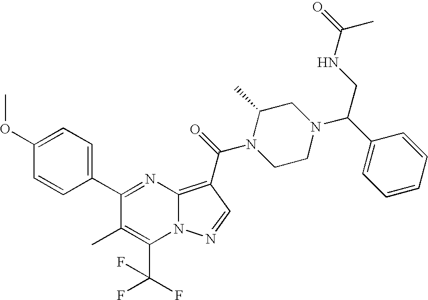 |
| Molecular Weight | 594.63 |
| Name | acetamide, N-[2-[(3R)-4-[[5-(4-methoxyphenyl)-6-methyl-7- |
| | (trifluoromethyl)pyrazolo[1,5-α]pyrimidin-3-yl]carbonyl]-3- |
| | methyl-1-piperazinyl]-2-phenylethyl]- |
| 6HB IC50 | +++ |
| Nl4 | +++ |
| Structure | 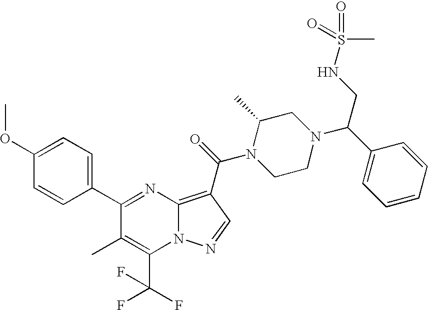 |
| Molecular Weight | 630.68 |
| Name | methanesulfonamide, N-[2-[(3R)-4-[[5-(4-methoxyphenyl)-6- |
| | methyl-7-(trifluoromethyl)pyrazolo[1,5-α]pyrimidin-3- |
| | yl]carbonyl]-3-methyl-1-piperazinyl]-2-phenylethyl]-, 2,2,2- |
| | trifluoroacetate |
| 6HB IC50 | +++ |
| Nl4 | +++ |
| Structure | 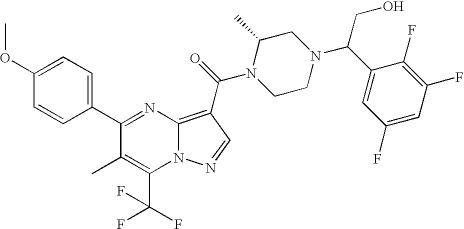 |
| Molecular Weight | 607.55 |
| Name | methanone, [(2R)-4-[2-hydroxy-1-(2,3,5-trifluorophenyl)ethyl]- |
| | 2-methyl-1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | +++ |
| Structure | 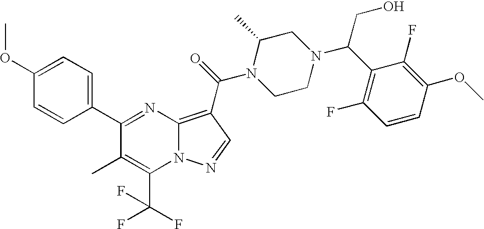 |
| Molecular Weight | 619.58 |
| Name | methanone, [(2R)-4-[1-(2,6-difluoro-3-methoxyphenyl)-2- |
| | hydroxyethyl]-2-methyl-1-piperazinyl][5-(4-methoxyphenyl)-6- |
| | methyl-7-(trifluoromethyl)pyrazolo[1,5-α]pyrimidin-3-yl]- |
| 6HB IC50 | +++ |
| Nl4 | +++ |
| Structure | 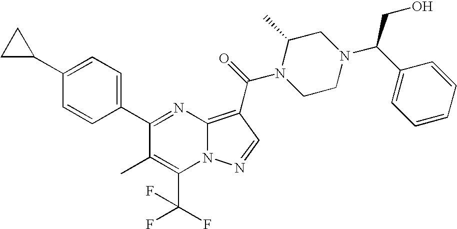 |
| Molecular Weight | 563.61 |
| Name | methanone, [5-(4-cyclopropylphenyl)-6-methyl-7- |
| | (trifluoromethyl)pyrazolo[1,5-α]pyrimidin-3-yl][(2R)-4-[(1R)- |
| | 2-hydroxy-1-phenylethyl]-2-methyl-1-piperazinyl]- |
| 6HB IC50 | +++ |
| Nl4 | +++ |
| Structure | 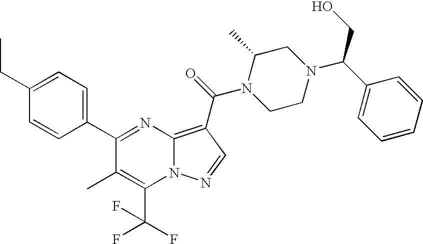 |
| Molecular Weight | 551.60 |
| Name | methanone, [5-(4-ethylphenyl)-6-methyl-7- |
| | (trifluoromethyl)pyrazolo[1,5-α]pyrimidin-3-yl][(2R)-4-[(1R)- |
| | 2-hydroxy-1-phenylethyl]-2-methyl-1-piperazinyl]- |
| 6HB IC50 | +++ |
| Nl4 | +++ |
| Structure | 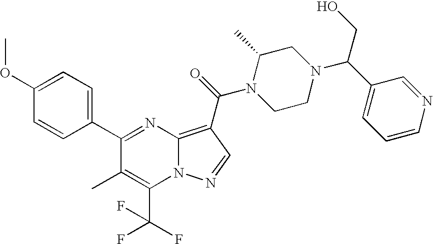 |
| Molecular Weight | 554.56 |
| Name | methanone, [(2R)-4-[2-hydroxy-1-(3-pyridinyl)ethyl]-2-methyl- |
| | 1-piperazinyl][5-(4-methoxyphenyl)-6-methyl-7- |
| | (trifluoromethyl)pyrazolo[1,5-α]pyrimidin-3-yl]- |
| 6HB IC50 | +++ |
| Nl4 | + |
| Structure | 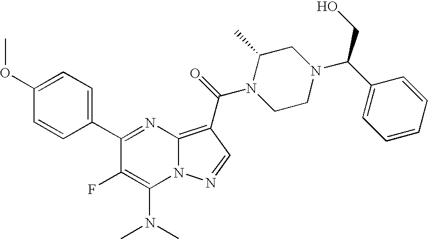 |
| Molecular Weight | 532.61 |
| Name | methanone, [7-(dimethylamino)-6-fluoro-5-(4- |
| | methoxyphenyl)pyrazolo[1,5-α]pyrimidin-3-yl][(2R)-4-[(1R)-2- |
| | hydroxy-1-phenylethyl]-2-methyl-1-piperazinyl]- |
| 6HB IC50 | +++ |
| Nl4 | +++ |

Compounds of the present invention include all regioisomers (e.g., cis and trans isomers) and stereoisomers (e.g. R and S enantiomers) of the compound of Formula I as well as racemic and diastereomeric forms of such isomers. The present invention includes an enantiomeric excess of any particular stereoisomer that is described herein. Enantiomeric excesses include excesses of whereby a particular enantiomer is provided in a relative percentage of 55%, 60%, 65%, 70%, 75%, 80%, 85%, 90% and 95% compared to its other enantiomer. The optical isomers can be obtained by resolution of the racemic mixtures according to conventional processes, for example by formation of diastereoisomeric salts by treatment with an optically active base and then separation of the mixture of diastereoisomers by crystallization, followed by liberation of the optically active bases from such salts. Alternatively, diastereoisomeric salts may be treated with an optically active acid and then separation of the mixture of diastereoisomers by crystallization, followed by liberation of the optically active acids from such salts. Examples of appropriate bases are brucine, dehydroabietylamine, quinine, cinchonidine, ephedrine, α-methylbenzylamine, deoxyphedrine, 2-amino-1-butanol, and 1-(1-naphthyl)ethylamine. Examples of appropriate acids are tartaric, diacetyltartaric, dibenzoyltartaric, ditoluoyltartaric and camphorsulfonic acid. A different process for separation of optical isomers involves the use of a chiral chromatography column optimally chosen to maximize the separation of the enantiomers. Still another available method involves synthesis of covalent diastereoisomeric molecules. In some methods, the synthesized diastereoisomers can be separated by conventional means such as chromatography, distillation, crystallization or sublimation, and then hydrolyzed to deliver the enantiomerically pure compound. The optically active compounds of the present invention can likewise be obtained by utilizing an optically active starting material or reagent. These isomers may be in the form of a free acid, a free base, an ester, a salt, an amide or a prodrug.

When any variable (e.g. R₇, heteroatom, X₂) occurs more than one time in any moiety, the choice of a variable is independently selected in each occurrence.

When present, any or all of alkyl, alkoxy, alkylthio, carboxamido, aryl, carbocycle, heterocyclyl, and heteroaryl radicals may be substituted at any position with a C₁-C₆ alkyl, hydroxyl, halo, amino, alkylamino, dialkylamino, carboxy, or cyano group so long as appropriate valences are maintained.

Some compounds of Formula I and their respective prodrugs can exist in several tautomeric forms, including the keto-enol form and enamine-imine form and geometric isomers and mixtures thereof. Even though one tautomer may be described, the present invention includes all tautomers of the present compounds.

### Unit Dosages

Dosages described in this application refer to mass of the free acid equivalent of the relevant compound.

Illustrative dosage unit forms of the pharmaceutical compositions can typically contain about, 100, 200, 250, 300, 350, 400, 450, or 500 mg of a compound of the present invention. In some embodiments, the dosage unit form contains about 200, 300, 400, or 500 mg of a compound of the present invention. The dosage unit form can be selected to accommodate the desired frequency of administration used to achieve the specified daily dosage. The amount of the unit dosage form of the pharmaceutical composition that is administered and the dosage regimen for treating the condition or disorder depends on a variety of factors, including the age, weight, sex and medical condition of the subject, the severity of the condition or disorder, the route and frequency of administration, and thus can vary widely, as is well known.

Where it is desired to formulate dosage units in which each unit consists of less than a therapeutically effective amount of a compound of the present invention, multiple dosage units, each containing smaller amounts of a compound of the present invention, can be administered to constitute the daily dose.

The amount of active ingredient that may be combined with the carrier materials to produce a single dosage form will vary depending upon the host treated and the particular mode of administration.

It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy.

### Prodrugs

The present invention further provides pharmaceutical compositions and methods of treatment comprising prodrugs of a compound of Formula I. Prodrugs of this invention may be called single, double, or triple, depending on the number of biotransformation steps required to release the active parent drug, and indicating the number of functionalities present in a precursor-type form. Prodrug forms often offer advantages of solubility, tissue compatibility, or delayed release in the mammalian organism. Prodrugs commonly known in the art include acid derivatives well known to practitioners of the art, such as, for example, an ester prepared by reaction of a parent acid with a suitable alcohol, or an amide prepared by reaction of the parent acid compound with an amine, or a basic group reacted to form an acylated base derivative. Moreover, the prodrug derivatives of this invention may be combined with other features herein taught to enhance bioavailability. For example, a compound of Formula I having one or more free amino, amido, hydroxy or carboxylic groups can be converted into prodrugs. Prodrugs include compounds comprising an amino acid residue, or a polypeptide chain of two or more amino acid residues which are covalently joined through peptide bonds to a free amino, hydroxy or carboxylic acid groups of compounds of the invention. Amino acid residues useful in accordance with the present invention include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, 4-hydroxyproline, hydroxylysine, demosine, isodemosine, 3-methylhistidine, 2-aminovaleric acid, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of a compound of the invention through the carbonyl carbon prodrug sidechain.

### Salts

The present invention further provides a pharmaceutically acceptable salt of a compound of the present invention composition.

The term “pharmaceutically acceptable salt” refers to a salt prepared from pharmaceutically acceptable non-toxic acids, including inorganic acids and organic acids.

Pharmaceutically acceptable salts of the compounds of the invention can be prepared by contacting the base forms of these compounds with a stoichiometric amount of the appropriate base or acid in an aqueous solvent, such as water, or in an organic solvent, or in a mixture of aqueous and organic solvents. In some embodiments of the present invention, nonaqueous media such as ether, ethyl acetate, ethanol, isopropanol, dichloromethane or acetonitrile are preferred.

The term “pharmaceutically acceptable salt” as used herein, refers to salts of the compounds of Formula I which are substantially non-toxic to living organisms. Typical pharmaceutically acceptable salts include those salts prepared by reaction of the compounds of the present invention with a pharmaceutically acceptable mineral or organic acid. Such salts are also known as acid addition salts. Such salts include the pharmaceutically acceptable salts listed in Journal of Pharmaceutical Science, 1955; 66:2 19, which are known to the skilled artisan.

Acids commonly employed to form acid addition salts are inorganic acids such as hydrochloric acid, hydrobromic acid, hydroiodic acid, sulfuric acid, and phosphoric acid, and organic acids such as p-toluenesulfonic, methanesulfonic acid, benzenesulfonic acid, oxalic acid, p-bromophenylsulfonic acid, carbonic acid, succinic acid, citric acid, benzoic acid, and acetic acid. Example of such pharmaceutically acceptable salts are the sulfate, pyrosulfate, bisulfate, sulfite, bisulfite, phosphate, monohydrogen phosphate, dihydrogen phosphate, metaphosphate, pyrophosphate, bromide, hydrobromide, iodide, acetate, propionate, decanoate, caprate, caprylate, acrylate, ascorbate, formate, hydrochloride, monohydrochloride, dihydrochloride, isobutyrate, caproate, heptanoate, propiolate, glucuronate, glutamate, propionate, phenylpropionate, salicylate, oxalate, malonate, succinate, suberate, sebacate, fumarate, malate, maleate, hydroxymaleate, mandelate, mesylate, nicotinate, isonicotinate, cinnamate, hippurate, nitrate, stearate, phthalate, terephthalate, butyne-1,4-dicarboxylate, benzoate, chlorobenzoate, methylbenzoate, hydroxybenzoate, methoxybenzoate, dinitrobenzoate, o-acetoxybenzoate, naphthalene-2-benzoate, phthalate, p-toluenesulfonate, p-bromobenzenesulfonate, p-chlorobenzenesulfonate, xylenesulfonate, phenylacetate, trifluoroacetate, phenylpropionate, phenylbutyrate, citrate, lactate, α-hydroxybutyrate, glycolate, tartrate, hemi-tartrate, benzenesulfonate, methanesulfonate, ethanesulfonate, propanesulfonate, hydroxyethanesulfonate, 1-naphthalenesulfonate, 2-naphthalenesulfonate, 1,5-naphthalenedisulfonate, mandelate, tartarate, and the like. In one embodiment, the pharmaceutically acceptable salt is a hydrochloride salt of a compound of the present invention. In one embodiment, the pharmaceutically acceptable salt is a hydrobromide salt of a compound of the present invention. In one embodiment, the pharmaceutically acceptable salt is a methanesulfonate salt of a compound of the present invention.

It should be recognized that any salt may exist as a hydrate or solvate and that such hydrates and solvates are contemplated by the present invention.

Multiple salts forms are included within the scope of the present invention where a chemical of the present invention contains more than one group capable of forming such a salt. In some embodiments, disalts are preferred. Examples of typical mono-salt forms include, but are not limited to hydrochloride and methanesulfonate. Examples of suitable multiple salt forms include, but are not limited to dihydrochloride and (bis)methanesulfonate.

For therapeutic uses, a salt of a compound of Formula I comprises a pharmaceutically acceptable counterion. However, non-pharmaceutically acceptable salts useful in the synthesis, preparation, or purification of a pharmaceutically acceptable compound are also embraced by the present invention.

The present invention further provides a pharmaceutical composition comprising a therapeutically effective amount of a compound of the present invention and one, two, three, four, five or six agents selected from the group consisting of a HIV protease inhibitor, a HIV reverse transcriptase inhibitor, an HIV entry inhibitor, an HIV fusion inhibitor, an HIV attachment inhibitor, an HIV integrase inhibitor and an HIV maturation inhibitor, and a pharmaceutically acceptable carrier.

The present invention further provides a pharmaceutical composition comprising a therapeutically effective amount of a compound of the present invention and a pharmaceutically acceptable carrier.

The present invention comprises a pharmaceutical composition for the treatment of retroviral disorders, such as HIV, comprising a therapeutically-effective amount of a compound of the present invention in association with at least one pharmaceutically-acceptable carrier, adjuvant or diluent.

The pharmaceutical compositions of the present invention comprise a compound of Formula I in association with one or more non-toxic, pharmaceutically-acceptable excipient. The excipients are acceptable in the sense of being compatible with the other ingredients of the composition and are not deleterious to the recipient. The pharmaceutical compositions of the present invention can be adapted for administration by any suitable route by selection of appropriate carrier materials and a dosage of a compound of the present invention effective for the treatment intended. For example, these compositions can be prepared in a form suitable for administration orally, intravascularly, intraperitoneally, subcutaneously, intramuscularly (IM) or rectally. Accordingly, the carrier material employed can be a solid or a liquid, or both, and is preferably formulated with the compound as a unit-dose composition, for example, a tablet, which can contain from about 1% to about 95%, preferably about 10% to about 75%, more preferably about 20% to about 60%, and still more preferably about 20% to about 40%, by weight of a compound of the present invention.

The compounds of the present invention may be administered orally, parenterally, sublingually, rectovaginally, topically, transmucosally, transdermally, or through liposomes in dosage unit formulations optionally comprising conventional nontoxic pharmaceutically acceptable carriers, adjuvants, or vehicles as desired.

“Formulations suitable for systemic administration” means formulations which are in a form suitable to be administered systemically to a patient. Systematic administration can be achieved by oral delivery, parenteral delivery, transmucosal delivery, transdermal delivery, rectovaginal delivery or liposomal delivery.

“Formulations suitable for oral administration” means formulations which are in a form suitable to be administered orally to a patient. In some embodiments, the oral formulation is intended to be absorbed in the gastric or intestinal cavities. The formulations may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste. Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coating. Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents. In some embodiments, the oral formulation is intended to be absorbed at least in part in the oral cavity including the lips, the inside lining of the lips and cheeks (buccal mucosa), the teeth, the gums (gingivae), the tongue, the floor of the mouth below the tongue, the bony roof of the mouth (hard palate), the area behind the wisdom teeth (retromolar trigone), and the salivary glands. Formulations suitable for topical administration in the mouth include lozenges comprising the active ingredient in a flavored basis, for example sucrose and acacia or tragacanth; pastilles comprising the active ingredient in an inert basis such as gelatin and glycerin, or sucrose and acacia; and mouthwashes comprising the active ingredient in a suitable liquid carrier.

“Formulations suitable for parenteral administration” means formulations which are in a form suitable to be administered parenterally to a patient. The term “parenteral” as used herein includes subcutaneous delivery, intravenous delivery, and intramuscular delivery. In some embodiments of the present invention, the formulations comprise emulsions, suspensions, aqueous or non-aqueous injection solutions. Injectable formulations, for example sterile injectable aqueous or oleagenous suspensions, may be formulated according to the known art using suitable dispersing or wetting agents and suspending agents, thickening agents, anti-oxidants, buffers, bacteriostats, and solutes which render the formulation isotonic. In preferred embodiments formulations suitable for parenteral administration have a pH adjusted to be compatible with the blood of the intended recipient. The sterile injectable formulation may also be a sterile injectable solution or suspension in a nontoxic parenterally acceptable diluent or solvent, for example, as a solution in 1,3-propanediol. Among the acceptable vehicles and solvents that may be employed are physiologically compatible buffers such as water, Hank's solution, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables. Some embodiments of the present invention comprise lyophilized formulations. In some embodiments of the present invention, the compounds are formulated in solid form and redissolved or suspended immediately prior to use.

“Formulations suitable for topical administration” means formulations which are in a form suitable to be administered topically to a patient. The formulation may be presented as a topical ointment, salve, powder, alcohol based gel, water based gel, or cream, as is generally known in the art, or incorporated into a matrix base for application in a patch, which would allow a controlled release of compound through the transdermal barrier. When formulated in an ointment, the active ingredients may be employed with either a paraffinic or a water-miscible ointment base. Alternatively, the active ingredients may be formulated in a cream with an oil-in-water cream base. In some embodiments, the transmucosal or transdermal formulation comprises a penetrant appropriate to the barrier to be permeated by at least one active ingredient of the formulation. Such penetrants are generally known in the art, and include, for example, bile salts and fusidic acid derivatives for transmucosal administration. In addition, detergents may be used to facilitate permeation.

“Formulations suitable for rectovaginal administration” means formulations which are in a form suitable to be administered to the rectum or vagina of a patient.

“Formulations suitable for rectal administration” means formulations which are in a form suitable to be administered rectally to a patient. The rectal formulation is preferably administered in the form of suppositories which can be prepared by mixing the compounds useful according to this invention with suitable non-irritating excipients or carriers such as cocoa butter, a poly(ethylene glycol) or a suppository wax, which are solid at ordinary temperatures but liquid at body temperature and therefore, melt in the rectum or vaginal cavity and release the active component.

“Formulations suitable for vaginal administration” means formulations which are in a form suitable to be administered vaginally to a patient. The formulation may be presented as pessaries, tampons, creams, gels, pastes, foams or spray formulations containing in addition to the active ingredient such carriers as are known in the art to be appropriate.

The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any nontoxic, physiologically acceptable and metabolizable lipid capable of forming liposomes can be used. The present compositions in liposome form can contain, in addition to the compound of the present invention, at least one additional compound selected from the group consisting of stabilizers, preservatives, and excipients. The preferred lipids are the phospholipids and phosphatidyl cholines (lecithins), both natural and synthetic.

### Form of Pharmaceutical Compositions

The pharmaceutical compositions of the present invention comprise a compound of the present invention in association with one or more non-toxic, pharmaceutically-acceptable carriers, excipients or adjuvants (collectively referred to herein as “carrier materials”). The carrier materials are acceptable in the sense of being compatible with the other ingredients of the composition and are not deleterious to the recipient. The pharmaceutical compositions of the present invention can be adapted for administration by any suitable route by selection of appropriate carrier materials and a dosage of a compound of the present invention effective for the treatment intended. For example, these compositions can be prepared in a form suitable for administration orally, intravascularly, intraperitoneally, subcutaneously, intramuscularly or rectally. Accordingly, the carrier material employed can be a solid or a liquid, or both, and is preferably formulated with the compound as a unit-dose composition, for example, a tablet, which can contain from about 1% to about 95%, preferably about 25% to about 70%, more preferably about 40% are to about 60%, and still more preferably about 20%, by weight of a compound of the present invention. Such pharmaceutical compositions of the invention can be prepared by any of the well known techniques of pharmacy, consisting essentially of admixing the components.

### Oral Administration

For oral administration, the pharmaceutical composition can contain a desired amount of a compound of the present invention of the present invention and be in the form of, for example, a tablet, a hard or soft capsule, a lozenge, a cachet, a dispensable powder, granules, a suspension, an elixir, a liquid, or any other form reasonably adapted for oral administration. Such a pharmaceutical composition is preferably made in the form of a discrete dosage unit containing a predetermined amount of a compound of the present invention, such as tablets or capsules. Such oral dosage forms can further comprise, for example, buffering agents. In some embodiments of the present invention, tablets, pills, or other solid dosage forms are prepared with enteric coatings. Unit dosage tablets or capsules are preferred.

Pharmaceutical compositions suitable for buccal or sub-lingual administration include, for example, lozenges comprising a compound of the present invention in a flavored base, such as sucrose, and acacia or tragacanth, and pastilles comprising a compound of the present invention in an inert base such as gelatin and glycerin or sucrose and acacia.

Liquid dosage forms for oral administration can include pharmaceutically acceptable emulsions, solutions, suspensions, syrups, and elixirs containing inert diluents commonly used in the art, such as water or a cyclodextrin. Such compositions can also comprise, for example, wetting agents, emulsifying and suspending agents, and sweetening, flavoring, and perfuming agents. Examples of suitable liquid dosage forms include, but are not limited, aqueous solutions comprising a compound of the present invention and β-cyclodextrin or a water soluble derivative of β-cyclodextrin such as sulfobutyl ether β-cyclodextrin, heptakis-2,6-di-O-methyl-α-cyclodextrin, hydroxypropyl-β-cyclodextrin, or dimethyl-β-cyclodextrin. Alternative liquid dosage forms comprise poly(ethylene glycol).

### Parenteral Administration

The pharmaceutical compositions of the present invention can also be administered by parenterally (subcutaneous, intravenous, or intramuscular). Such injectable compositions can employ, for example, saline, dextrose, or water as a suitable carrier material. The pH value of the composition can be adjusted, if necessary, with suitable acid, base, or buffer. Suitable bulking, dispersing, wetting or suspending agents, including mannitol and poly(ethylene glycol)s, for example PEG400, can also be included in the composition. A suitable parenteral composition can also include a compound of the present invention in injection vials. Aqueous solutions can be added to dissolve the composition prior to injection.

### Rectovaginal Administration

The pharmaceutical compositions can be rectally or vaginally. Illustrative pharmaceutical compositions are administered in the form of a suppository or a pessary. In some embodiments, the rectovaginal formulations comprise a compound of the present invention in a total amount of, for example, 0.075 to 30% w/w, preferably 0.2 to 20% w/w and most preferably 0.4 to 15% w/w. Carrier materials such as cocoa butter, theobroma oil, and other oil and poly(ethylene glycol) suppository bases can be used in such compositions. Other carrier materials such as coatings, for example, hydroxypropyl-methylcellulose film coating, and disintegrants, for example, croscarmellose sodium and cross-linked povidone are also contemplated as part of the present invention.

As indicated above, these pharmaceutical compositions can be prepared by any suitable method of pharmacy which includes the step of bringing into association a compound of the present invention and at least one carrier material. In general, the compositions are prepared by uniformly and intimately admixing the active compound with a liquid or finely divided solid carrier, or both, and then, optionally coating the admixture, and then, optionally shaping the product. For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binding agent, lubricant, inert diluent or surface active/dispersing agent. Molded tablets can be made by molding, in a suitable machine, the powdered compound moistened with an inert liquid diluent.

### Carrier Materials

As noted above, for therapeutic purposes, the pharmaceutical compositions of the present invention comprise a compound of the present invention in a desired amount in combination with at least one pharmaceutically-acceptable carrier material appropriate to the indicated route of administration. It is understood in the art that certain carrier materials may provide a plurality of functions, for example hydroxypropylmethylcellulose may function as both a water retention agent and as an emulsifier; as such the inclusion of any particular excipient as a member of one class is not intended to limit other classes to its exclusion.

Oral dosage forms of the pharmaceutical compositions of the present invention preferably comprise a compound of the present invention in a desired amount admixed with one or more carrier materials selected from the group consisting of diluents, disintegrants, binding agents and adhesives, wetting agents, lubricants, and anti-adherents. More preferably, such compositions are tableted or encapsulated for convenient administration.

Injectable dosage forms preferably are adapted for parenteral injection. Preferably, these dosage forms comprise a compound of the present invention in aqueous or non-aqueous isotonic sterile injection solutions or suspensions, such as a of a compound of the present invention suspended or dissolved in water, poly(ethylene glycol), propylene glycol, ethanol, corn oil, cottonseed oil, peanut oil, sesame oil, benzyl alcohol, or other pharmaceutically acceptable buffers. These solutions and suspensions can be prepared from sterile powders or granules having one or more of the carriers or diluents mentioned for use in the formulations for oral administration.

The selection and combination of carrier materials used in the pharmaceutical compositions of the present invention provides compositions exhibiting improved performance with respect to, among other properties, safety, efficacy, dissolution profile, disintegration profile, bioavailability, clearance times, stability, pharmacokinetic properties and pharmacodynamic properties. The carrier materials preferably are water soluble or water dispersible and have wetting properties to increase the aqueous solubility and decrease the hydrophobicity of pharmaceutical compositions of the present invention. Where the composition is formulated as a tablet, the combination of carrier materials selected provides tablets that can exhibit, among other properties, improved dissolution and disintegration profiles, hardness, crushing strength, or friability properties.

### Diluents

The pharmaceutical compositions of the present invention optionally can comprise one or more diluents as a carrier material. Suitable diluents can include, either individually or in combination, such diluents as lactose USP; lactose USP, anhydrous; lactose USP, spray dried; starch USP; directly compressible starch; mannitol USP; sorbitol; dextrose monohydrate; microcrystalline cellulose NF; dibasic calcium phosphate dihydrate NF; sucrose-based diluents; confectioner's sugar; monobasic calcium sulfate monohydrate; calcium sulfate dihydrate NF; calcium lactate trihydrate granular NF; dextrates NF, for example Emdex™; Celutab™; dextroses, for example Cerelose™; inositol; hydrolyzed cereal solids such as the Maltrons™ and Mor-Rex™; amylose; Rexcel™; powdered celluloses, for example Elcema™; calcium carbonate; glycine; bentonite; and polyvinylpyrrolidone. The present pharmaceutical compositions comprise one or more diluents in the range of about 5% to about 99%, preferably about 25% to about 90%, and more preferably about 40% to about 80%, of the total weight of the composition. The selected diluent or diluents preferably exhibit suitable compressibility and pre-compression flow properties. Microcrystalline celluloses, for example Avicel™ PH 101 and lactose, either individually or in combination are preferred diluents. The use of extragranular microcrystalline cellulose, for example microcrystalline cellulose added to a wet granulated composition after the drying step, in addition to intragranular microcrystalline cellulose, for example microcrystalline cellulose added to the composition during or before the wet granulation step, can be used to improve tablet hardness or disintegration time. Lactose, especially lactose monohydrate, is particularly preferred. Lactose typically provides pharmaceutical compositions having suitable release rates, stability, pre-compression flowability, and drying properties at a relatively low diluent cost.

### Disintegrants

The pharmaceutical compositions of the present invention optionally can comprise one or more disintegrants as a carrier material, particularly for tablet formulations. Suitable disintegrants can include, either individually or in combination, such disintegrants as starches; sodium starch glycolate; clays, for example Veegum™ HV; celluloses, for example purified cellulose, methylcellulose, sodium carboxymethylcellulose, or carboxymethylcellulose; alginates; pregelatinized corn starches, for example National™ 1551, or National™ 1550; crospovidone USP NF; gums, for example agar, guar, locust bean, Karaya™, pectin, or tragacanth. Disintegrants can be added at any suitable step during the preparation of the pharmaceutical composition, particularly prior to granulation or during the lubrication step prior to compression. The present pharmaceutical compositions comprise one or more disintegrants in the range of about 0.5% to about 30%, preferably about 1% to about 10%, and more preferably about 2% to about 6%, of the total weight of the composition. Croscarmellose sodium is a preferred disintegrant for tablet formulations, preferably in the range of about 1% to about 10%, preferably about 2% to about 6%, and more preferably about 5%, by weight of the composition.

### Binding Agents and Adhesives

The pharmaceutical compositions of the present invention optionally can comprise one or more binding agents or adhesives as a carrier material. Such binding agents and adhesives preferably impart sufficient cohesion to the powders to permit normal processing such as sizing, lubrication, compression and packaging, but still permit the tablet to disintegrate and the composition to dissolve upon ingestion. Suitable binding agents and adhesives include, either individually or in combination, such binding agents and adhesives as acacia; tragacanth; sucrose; gelatin; glucose; starch; cellulose materials such as, but not limited to, methylcellulose, or sodium carboxymethylcellulose, for example Tylose™; alginic acid; salts of alginic acid; magnesium aluminum silicate; poly(ethylene glycol); guar gum; polysaccharide acids; bentonites; polyvinylpyrrolidone (povidone); polymethacrylates; hydroxypropylmethylcellulose (HPMC); hydroxypropyl-cellulose, for example Klucel™; ethyl cellulose, for example Ethocel™; pregelatinized starch, for example National™ 1511 or Starch 1500. In some embodiments, pharmaceutical compositions of the present invention comprise one or more binding agents or adhesives in the range of about 0.5% to about 25%, preferably about 0.75% to about 15%, and more preferably about 1% to about 10%, of the total weight of the composition.

### Wetting Agents

Where it is desired to increase the aqueous solubility of a compound of the present invention, the pharmaceutical compositions can optionally comprise one or more wetting agents as a carrier material, particularly for tablet formulations. Such wetting agents preferably maintain the compound in solution and improve the bioavailability of the pharmaceutical composition. Suitable wetting agents include, either individually or in combination, such wetting agents as oleic acid; glyceryl monostearate; sorbitan monooleate; sorbitan monolaurate; triethanolamine oleate; polyoxyethylene sorbitan monooleate; polyoxyethylene sorbitan monolaurate; sodium oleate; and sodium lauryl sulfate. In some embodiments, wetting agents that are surfactants are preferred. In some embodiments, wetting agents that are anionic surfactants are preferred. The present pharmaceutical compositions comprise one or more wetting agents present at about 0.1% to about 15%, preferably about 0.25% to about 10%, and more preferably about 0.5% to about 5%, of the total weight of the composition. Sodium lauryl sulfate is a preferred wetting agent for tablet formulations. The compositions of the present invention preferably comprise sodium lauryl sulfate as the wetting agent at about 0.25% to about 7%, more preferably about 0.4% to about 4%, and still more preferably about 0.5 to about 2%, of the total weight of the composition.

### Lubricants

The pharmaceutical compositions of the present invention optionally comprise one or more lubricants as a carrier material. Suitable lubricants include, either individually or in combination, glyceryl behenate, for example Compritol™ 888; metallic stearates, for example magnesium, calcium and sodium stearates; stearic acid; hydrogenated vegetable oils, for example Sterotex™; talc; waxes; Stearowet™; boric acid; sodium benzoate and sodium acetate; sodium chloride; DL-leucine; poly(ethylene glycol)s, for example Carbowax™ 4000 and Carbowax™ 6000; sodium oleate; sodium benzoate; sodium acetate; sodium lauryl sulfate; sodium stearyl fumarate, for example Pruv™; and magnesium lauryl sulfate. The present pharmaceutical compositions comprise one or more lubricants at about 0.1% to about 10%, preferably about 0.2% to about 8%, and more preferably about 0.25% to about 5%, of the total weight of the composition. In some embodiments magnesium stearate is a lubricant used to reduce friction between the equipment and granulation during compression.

### Anti-Adherents or Glidants

The pharmaceutical compositions of the present invention optionally can comprise one or more anti-adherent agents or glidants as a carrier material. Suitable anti-adherents or glidants include, either individually or in combination, such anti-adherents as talc, cornstarch, Cab-O-Sil™, Syloid™, DL-leucine, sodium lauryl sulfate, and metallic stearates. The present pharmaceutical compositions comprise one or more anti-adherents or glidants at about 0.1% to about 15%, preferably about 0.25% to about 10%, and more preferably about 0.5% to about 5%, of the total weight of the composition. Talc is a preferred anti-adherent or glidant agent used to reduce formulation sticking to equipment surfaces and also to reduce static in the blend. The compositions preferably comprise talc at about 0.1% to about 10%, more preferably about 0.25% to about 5%, and still more preferably about 0.5% to about 2%, of the total weight of the composition.

Other carrier materials, for example colorants, flavors and sweeteners can be used in the preparation of the pharmaceutical compositions of the present invention.

Oral dosage forms, including tablets, can be coated or uncoated.

The individual pharmaceutically acceptable carrier materials described in the above embodiment optionally can be replaced with other suitable carrier materials if desired. Acceptable substitute carrier materials are chemically compatible both with the compound of the present invention and with the other carrier materials.

Compounds of the present invention can be used in the treatment of HIV in patients who are not adequately treated by other HIV-1 therapies. Accordingly, the invention is also drawn to a method of treating a patient in need of therapy, wherein the HIV-1 infecting said cells does not respond to at least one other HIV-1 therapy. In some embodiments, methods of the invention are administered to a patient infected with an HIV that is resistant to at least one class of drugs approved to treat HIV infection. In various applications, the HIV is resistant to one or more protease inhibitors, reverse transcriptase inhibitors, entry inhibitors, nucleoside analogs, vaccines, fusion inhibitors, attachment inhibitors, CCR5 inhibitors, CXCR4 inhibitors, CCR5 antibodies, CXCR4 antibodies, integrase inhibitors, and immunomodulators. In some embodiments, methods of the invention are administered to a patient infected with an HIV that is resistant to at least one drug approved to treat HIV infection. In some embodiments, the compositions and methods of the invention are practiced on a subject infected with an HIV that is resistant to one or more drugs used to treat HIV infections, for example, but not limited to, zidovudine, lamivudine, didanosine, zalcitabine, stavudine, abacavir, nevirapine, delavirdine, emtricitabine, efavirenz, saquinavir, ritonavir, lopinavir, indinavir, nelfinavir, tenofovir, amprenavir, adefovir, atazanavir, fosamprenavir, enfuvirtide, tipranavir, darunavir, maraviroc, elvitegravir, raltegravir, TMC-125, TMC-278, hydroxyurea, AL-721, ampligen, butylated hydroxytoluene, polymannoacetate, castanospermine, contracan, creme pharmatex, CS-87, penciclovir, famciclovir, acyclovir, cytofovir, ganciclovir, dextran sulfate, D-penicillamine, trisodium phosphonoformate, fusidic acid, HPA-23, eflomithine, nonoxynol, pentamidine isethionate, peptide T, phenyloin, isoniazid, ribavirin, rifabutin, ansamycin, trimetrexate, SK-818, suramin, UA001, and combinations thereof.

In addition, a compound of the present invention can be used as a prophylactic to prevent transmission of HIV infection between individuals. For example, a compound of the present invention can be administered orally or by injection to an HIV infected pregnant woman or her fetus during pregnancy, immediately prior to, at, or subsequent to birth, to reduce the probability that the newborn infant becomes infected. Also, a compound of the present invention can be administered vaginally immediately prior to childbirth to prevent infection of the infant during passage through the birth canal. Further, a compound of the present invention can be used during sexual intercourse to prevent transmission of HIV by applying a retroviral inhibiting effective amount of a topical composition comprising a compound of the present invention to vaginal or other mucosa prior to sexual intercourse.

Various dosage amounts of the composition of the invention can be administered to provide various plasma levels of a compound of the present invention. In some embodiments, a preferred dosage amount is one which provides a trough concentration of a compound of the present invention in the patient's plasma of about 1 micromolar (μM) to about 1 millimolar (mM). In some embodiments, the dosage amount is one which provides a trough concentration of a compound of the present invention in the patient's plasma of about 500 nM to about 1000 μM, about 1 μM to about 500 μM, or about 40 μM to about 250 μM. In some embodiments, the dosage amount is one which provides a trough concentration of a compound of the present invention in the patient's plasma of about 750 nM to about 200 μM, about 1 μM to about 100 μM, or about 40 μM to about 75 μM. In some embodiments, the dosage amount is one which provides a trough concentration of a compound of the present invention in the patient's plasma of at least about 4 μM or greater, at least about 10 μM or greater, at least about 40 μM or greater, at least about 100 μM or greater, or at least 200 μM or greater. In some embodiments, the dosage amount is one which provides a trough concentration of a compound of the present invention in the patient's plasma of about 400 μM. The “trough concentration” is the concentration of a compound of the present invention in the patient's plasma just prior to subsequent dosing of the patient.

Therapeutic administration can also include prior, concurrent, subsequent or adjunctive administration of at least one compound of the present invention according to the present invention or other therapeutic agent, such as an anti-viral or immune stimulating agent. In such an approach, the dosage of the second drug can be the same as or different from the dosage of the first therapeutic agent. In one embodiment of the present invention, the drugs are administered on alternate days in the recommended amounts of each drug.

Administration of a compound of the present invention can also optionally include previous, concurrent, subsequent or adjunctive therapy using immune system boosters or immunomodulators. In addition to the pharmacologically active compounds, a pharmaceutical composition of the present invention can also contain suitable pharmaceutically acceptable carriers comprising excipients and auxiliaries which facilitate processing of the active compounds into preparations which can be used pharmaceutically. In one embodiment, the preparations, particularly those preparations which can be administered orally, such as tablets, dragees, and capsules, and also preparations which can be administered rectally, such as suppositories, as well as suitable solutions for administration by injection or orally, contain from about 0.01 to 99 percent of the active ingredient together with the excipient. In another embodiment, the preparation can include from about 20 to 75 percent of active compound(s), together with the excipient.

Pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture, and processing the mixture of granules, after adding suitable auxiliaries, if desired or necessary, to obtain tablets or dragee cores.

The present invention also provides all pharmaceutically-acceptable isotopically labeled compounds of the present invention wherein one or more atoms are replaced by atoms having the same atomic number, but an atomic mass or mass number different from the atomic mass or mass number predominantly found in nature.

Examples of isotopes suitable for inclusion in the compounds of the present invention include isotopes of hydrogen, for example ²H or ³H, carbon, for example ¹¹C, ¹³C, or ¹⁴C, chlorine, for example ³⁶Cl, fluorine, for example ¹⁸F, iodine, for example ¹²³I or ¹²⁵I, nitrogen, for example ¹³N or ¹⁵N, oxygen, for example ¹⁵O, ¹⁷O, or ¹⁸O, phosphorus, for example ³²P, and sulfur, for example ³⁵S.

Certain isotopically labeled compounds of the present invention are useful in drug or substrate tissue studies. The radioactive isotopes tritium (³H) and carbon-14 (¹⁴C) are particularly useful for this purpose in view of their ease of incorporation and ready means of detection.

Substitution with heavier isotopes, for example deuterium (²H) may afford certain therapeutic advantages resulting from greater metabolic stability, for example, increased in vivo half life or reduced dosage requirements.

Substitution with positron emitting isotopes, for example ¹¹C, ¹⁸F, ¹⁵O, or ¹³N, may be useful in positron emission topography (PET) studies for examining substrate-receptor occupancy.

The present invention also provides pharmaceutically acceptable solvates where the solvent of crystallization may be isotopically substituted, for example D₂O, acetone-d₆, or DMSO-d₆.

Isotopically labeled compounds of the present invention can be prepared by conventional techniques known to those skilled in the art or by synthetic processes analogous to those described in the present application using appropriate isotopically labeled reagents in place of the non-labeled reagent mentioned therein.

The present compounds may also be used in co-therapies, partially or completely, in place of other conventional antiviral therapies, such as in a combination comprising a first compound of the present invention and a second pharmaceutical agent selected from a second compound of the present invention or another anti-infective agent

In some embodiments of the present invention, combinations comprising a compound of the present invention in combination with another anti-infective agent will produce a synergistic effect or reduce the toxic side effects associated with another anti-infective by reducing the therapeutic dose of the side effect-causing agent needed for therapeutic efficacy or by directly reducing symptoms of toxic side effects caused by the side effect-causing agent.

Some embodiments of the present invention comprise a combination of a compound of the present invention and a secondary pharmaceutical agent selected from the group consisting of fusion inhibitors, entry inhibitors, reverse transcriptase inhibitors, attachment inhibitors, integrase inhibitors, protease inhibitors, assembly inhibitors, budding inhibitors, and maturation inhibitors in amounts effective for treatment of HIV when used in a combination therapy.

Some embodiments of the present invention comprise a compound of the present invention and in combination with an antiretroviral agent selected from the group consisting of vaccines, gene therapy treatments, cytokines, TAT inhibitors, and immunomodulators in amounts effective for treatment of HIV when used in a combination therapy.

Some embodiments of the present invention comprise a compound of the present invention and an anti-infective agent selected from the group consisting of antifungals, antibacterials, anti-neoplastics, anti-protozoals, DNA polymerase inhibitors, DNA synthesis inhibitors, anti-HIV antibodies, HIV antisense drugs, IL-2 agonists, α-glucosidase inhibitors, purine nucleoside phosphorylase inhibitors, apoptosis agonists, apoptosis inhibitors, and cholinesterase inhibitors, where the compounds are present in amounts effective for treatment of HIV when used in a combination therapy.

Some embodiments of the present invention comprise a compound of the present invention and a protease inhibitor selected from the group consisting of ritonavir, lopinavir, saquinavir, amprenavir, fosamprenavir, nelfinavir (AG1343), tipranavir, indinavir, atazanavir, TMC-125, TMC-278, darunavir, mozenavir, JE-2147 (AG1776), L-756423, KNI-272, DPC-681, DPC-684, telinavir (SC-52151), BMS 186318, droxinavir (SC-55389a), DMP-323, KNI-227, 1-[(2-hydroxyethoxy)methyl]-6-(phenylthio)thymine, AG-1859, RO-033-4649, R-944, DMP-850, DMP-851, and brecanavir. Preferred protease inhibitors for use in combination with a compound of the present invention include saquinavir, ritonavir, indinavir, nelfnavir, amprenavir, lopinavir, atazanavir, darunavir, brecanavir, fosamprenavir, and tipranavir.

Some embodiments of the present invention comprise a compound of the present invention and a reverse transcriptase inhibitor selected from the group consisting of emtricitabine, capravirine, tenofovir, lamivudine, zalcitabine, delavirdine, nevirapine, didanosine, stavudine, abacavir, alovudine, zidovudine, racemic emtricitabine, emivirine, elvucitabine, brecanavir, DPC-083, amdoxovir, MIV-210 (FLG), DFC (dexelvucitabine), dioxolane thymidine, Calanolide A, etravirine (TMC-125), L697639, atevirdine (U87201E), MIV-150, GSK-695634, GSK-678248, TMC-278, KP1461, KP-1212, lodenosine (FddA), 5-[(3,5-dichlorophenyl)thio]-4-isopropyl-1-(4-pyridylmethyl)imidazole-2-methanol carbamic acid, (−)-β-D-2,6-diaminopurine dioxolane, AVX-754, BCH-13520, BMS-56190 ((4S)-6-chloro-4-[(1E)-cyclopropylethenyl]-3,4-dihydro-4-trifluoromethyl-2-(1H)-quinazolinone), TMC-120, and L697639, where the compounds are present in amounts effective for treatment of HIV when used in a combination therapy.

Some embodiments of the present invention comprise a compound of the present invention and second anti-infective selected from the group consisting of maraviroc, elvitegravir, raltegravir, TMC-125, and TMC-278, where the compounds are present in amounts effective for treatment of HIV when used in a combination therapy.

Some embodiments of the present invention comprise a compound of the present invention and second anti-infective selected from the group consisting of interferon-alpha, pegylated interferon, ribavirin, telapravir, entecavir, and adefovir

Some embodiments of the present invention comprise a compound of the present invention and a viral entry inhibitor in amounts effective for treatment of HIV when used in a combination therapy. In some embodiments, the viral entry inhibitor is an attachment inhibitor. In some embodiments, the viral entry inhibitor is a fusion inhibitor. In some embodiments, the viral entry inhibitor is a CD4 receptor binding inhibitor. In some embodiments, the viral entry inhibitor is a CD4 mimic. In some embodiments, the viral entry inhibitor is a gp120 mimic. In some embodiments, the viral entry inhibitor is a gp41 antagonist. In some embodiments, the viral entry inhibitor is a CD4 monoclonal antibody. In some embodiments, the viral entry inhibitor is a CCR5 antagonist. In some embodiments, the viral entry inhibitor comprises a sub-class of CCR5 antagonists, for example a zinc finger inhibitor. In some embodiments, the viral entry inhibitor is a CXCR4 coreceptor antagonist.

Some embodiments of the present invention comprise a compound of the present invention and an immunomodulator is selected from the group consisting of pentamidine isethionate, autologous CD8+ infusion, α-interferon immunoglobulins, thymic peptides, IGF-1, anti-Leu3A, autovaccination, biostimulation, extracorporeal photophoresis, cyclosporin, rapamycin, FK-565, FK-506, GCSF, GM-CSF, hyperthermia, isopinosine, IVIG, HIVIG, passive immunotherapy, and polio vaccine hyperimmunization, where the compounds are present in amounts effective for treatment of HIV when used in a combination therapy.

Some embodiments of the present invention comprise a compound of the present invention and a secondary pharmaceutical agent selected from the group consisting of antifungals, antibacterials, anti-neoplastics, anti-protozoals, ceragenins, DNA polymerase inhibitors, DNA synthesis inhibitors, anti-HIV antibodies, HIV antisense drugs, IL-2 agonists, α-glucosidase inhibitors, purine nucleoside phosphorylase inhibitors, apoptosis agonists, apoptosis inhibitors, and cholinesterase inhibitors in amounts effective for treatment of HIV when used in a combination therapy.

### Activity Assays

### Generation of Chronically Infected H9 Cells:

3×10⁶H9 cells were incubated with 1 mL of HIV-1 (strain RF) supplemented with 40 μg/mL of DEAE-dextran. Virus and cells were incubated together at 37° C. for 2-4 hours with sporadic shaking to resuspend cells. 10 mL of media (RPMI 1640 containing 10% fetal bovine serum and supplemented with 50 μg/mL gentamicin) was then added and the virus-cell coculture was incubated at 37° C. in a humidified 5% CO₂ incubator. 3 days post-infection and every 2-3 days thereafter, the infected H9/HIV-1_{RF} cells were centrifuged, the supernatant was removed, and the cells were resuspended at a density of 0.3×10⁶ cells/mL in media (RPMI 1640 containing 10% fetal bovine serum and supplemented with 50 μg/mL gentamicin). Chronically infected H9/HIV-1_{RF} cells were used in experiments starting 12-15 days after infection and up to 40 days post-infection.

### Indicator Cells for Virus Infection:

TZM-b1 indicator cells were propagated in Dulbecco's Modified Eagle's Medium (DMEM) containing 10% fetal bovine serum (FBS, heat-inactivated) and supplemented with gentamicin (50 ug/mL).

### Six-Helix Bundle (6HB) Assay:

This assay determines the effect of compounds on conformational changes in HIV envelope glycoprotein (Env) that are required in order for Env to mediate the fusion of the viral and cellular membranes during virus entry and infection. Specifically, this assay measures the effect of compounds on the formation of the HIV Env six-helix bundle (6HB) structure.

H9 cells chronically infected with HIV-1 (H9/HIV-1_{RF}) were resuspended in Stain/Wash Buffer (1% bovine serum albumin, 0.1% sodium azide in phosphate-buffered saline) and aliquoted at 2.5×10⁵ cells per well into 96-well V-bottom plates containing various concentrations of test compounds. Cells and compounds were incubated for 30 minutes at 37° C. Recombinant soluble CD4 (sCD4) was then added to a final concentration of 1 μg/mL. Negative control wells contained no sCD4 and no test compound. Positive control wells contained sCD4 but no test compound. The plate was incubated for 1 hour at 37° C. to allow the sCD4 to bind to HIV Env and induce conformational changes in the Env protein. Antibody that is specific for the HIV Env six-helix bundle conformation was then added (1 μL polyclonal rabbit serum per well), and the plate was incubated for an additional 1 hour at 37° C. to permit antibody binding. The cells were then washed once with Stain/Wash Buffer to remove compound and excess antibody. The cells were resuspended in Stain/Wash Buffer containing PE-labeled anti-rabbit secondary antibody (KPL) at 0.25 μg per well. The cells were incubated for 45 min at 4° C. to permit secondary antibody binding. Fluorescence was detected using a BD FACSArray™ bioanalyzer. Compounds that inhibited Env conformational changes were identified as those that decreased primary antibody binding to the six-helix bundle epitope resulting in a decrease in fluorescence signal.

### Virus Infection Assay:

This assay determines the effects of compounds on virus entry and single-cycle infection of cells. In this assay, virus (HIV-1, NL4-3 strain) is added to indicator cells expressing the CD4 receptor and both the CXCR4 and CCR5 co-receptor. The cells contain both a lac Z and a luciferase reporter gene under control of the viral LTR transcription promoter. The cells and virus are incubated together in the presence of various concentrations of test compounds. Successful infection of the cells by the virus permits activation of the reporter gene by the viral Tat transcription factor. The resulting beta-galactosidase or luciferase activity is quantitated using a chemiluminescent substrate and a luminescent plate reader.

Specifically, one day prior to infection, 1.5×10⁴ TZM-b1 indicator cells were added to each well of a 96-well microtiter plate. On the day of the experiment, virus (MOI 0.01) in 50 μL of medium (Dulbecco's Modified Eagle's Medium) supplemented with DEAE-dextran at a final concentration of 80 μg/mL was added to cells along with various concentrations of test compound in 50 μL of Dulbecco's Modified Eagle's Medium. Control wells had virus alone. Following a 4 hr incubation at 37° C., complete media [DMEM supplemented with 10% fetal bovine serum (FBS, heat-inactivated) and gentamicin (50 ug/mL)] was added. One day post-infection the level of virus infection was quantitated by detection of β-galactosidase expression using the Gal-Screen system (Applied Biosystems).

### Synthetic Processes

The various groups depicted in the schemes are as defined above for the compounds of Formula I and the sub-generic formulae. The group R₈ refers to an optionally substituted phenyl group or optionally substituted pyrid-3-yl group; and the variable “n” is 1, 2 or 3. The group R₉ in the schemes below refers to a carboxy protecting group, such groups being well known in the art. The groups R₁₁ and R₁₂ in the schemes below refer to hydrogen or C₁-C₄ alkyl.

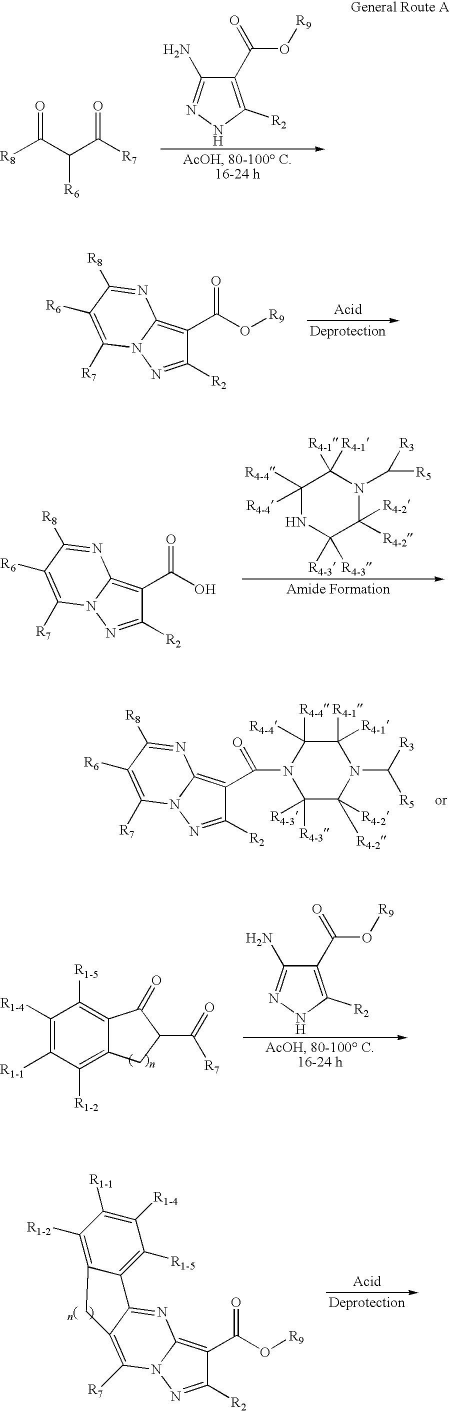

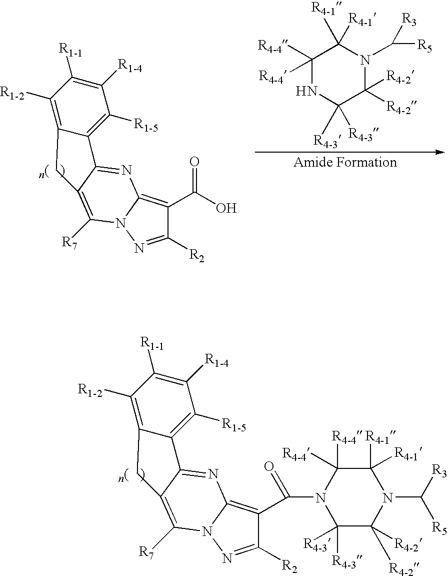

### Description of Methods Used in General Route A

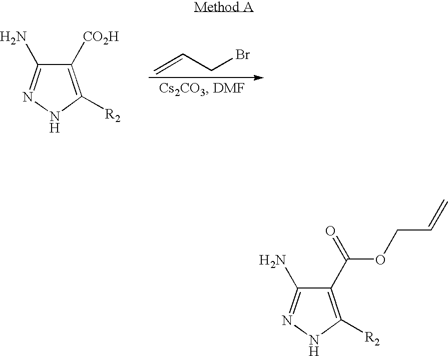

The 3-amino-1H-pyrazole-4-carboxylic acid (1 equiv) in DMF (20 vol) was treated with cesium carbonate (1.2 equiv) followed by allyl bromide (1.1 equiv) and stirred at rt for 16 h. Reaction progress was monitored by LC/MS. On completion the reaction was diluted with water (20 vol) and extracted into EtOAc (3×20 vol). The combined organic phases were washed with water (2×20 vol) and brine (20 vol), dried (Na₂SO₄), filtered and evaporated to give a residue that was purified by column chromatography (silica gel, 0-5% MeOH in DCM) providing the desired product.

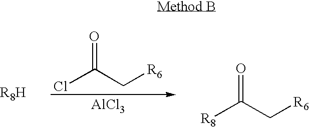

A solution of AlCl₃ (1 equiv) in trichloroethylene (16 vol) was cooled to 0° C. and treated with an acid chloride (1 equiv). The reaction was cooled to −50° C. and the substituted benzene (1 equiv) added dropwise over 5 min. Stirring was continued for 1 h. The mixture was poured onto ice/2 M HCl (30 vol) and extracted with EtOAc (2×30 vol). The combined organic layers dried (MgSO₄), filtered and evaporated to give the desired product.

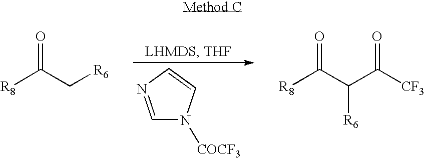

A solution of the ketone (1 equiv) in THF (20 vol) was added dropwise to a separate solution of LHMDS (2 equiv) in THF (30 vol) at −78° C. and stirred for 1 h at this temperature. 1-(Trifluoroacetyl)imidazole (2-4 equiv) was added dropwise and the reaction stirred for 1 h at −78° C. and 5 h at rt. The reaction mixture was poured onto ice (50 vol) and acidified to pH 1 with 1.2 M HCl. The aqueous solution was extracted with EtOAc (3×60 vol) and the combined organic layers washed with cold brine (1×60 vol), dried (Na₂SO₄), filtered and evaporated. The residue obtained was purified by column chromatography (silica gel, EtOAc in heptanes) to afford the desired product.

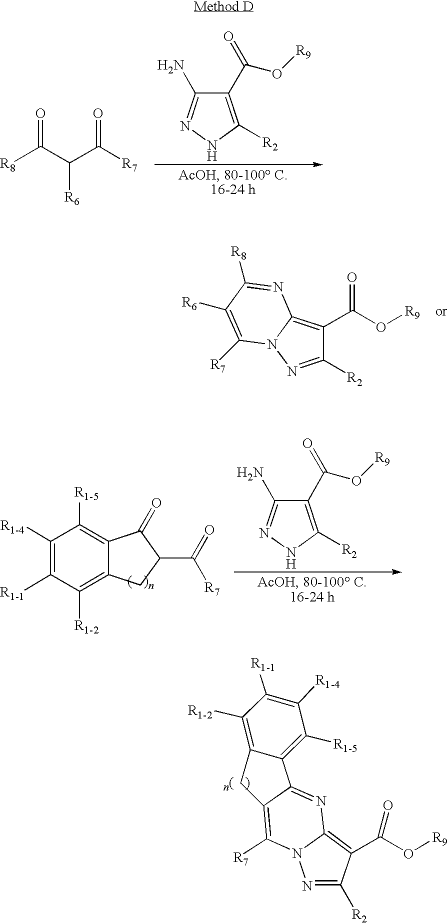

The 3-aminopyrazole ester (1.33-1.66 equiv) and diketone (1 equiv) in AcOH (10 vol) was heated at 80-100° C. for 16-24 h. Reaction progress was monitored by LC/MS. On completion the reaction mixture was diluted with water (15 vol) and the resultant precipitate isolated by filtration and dried under vacuum. Where necessary, column chromatography (silica gel, EtOAc in heptanes) was employed to obtain the desired product.

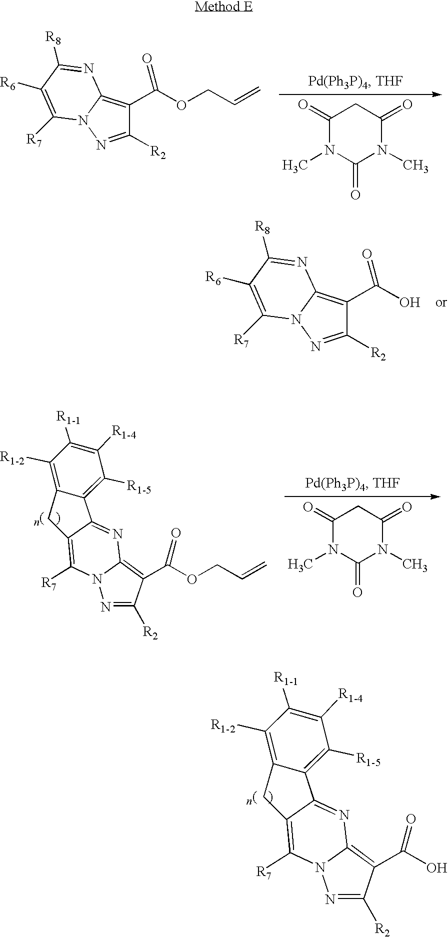

A solution of pyrazolopyrimidine allyl ester (1 equiv) and 1,3-dimethylbarbituric acid (1 equiv) in THF (20 vol) was degassed with N₂ for 5-10 min after which Pd(PPh₃)₄ (0.1 equiv) was added and the reaction stirred at rt for 2 h. Reaction progress was monitored by LC/MS. On completion the reaction mixture was diluted with water (20 vol) and extracted into EtOAc (3×20 vol). The combined organic phases were washed with water (2×20 vol) and brine (20 vol), dried (Na₂SO₄), filtered and evaporated to give a residue that was triturated with DCM (8.5 vol), filtered, and dried providing the desired product.

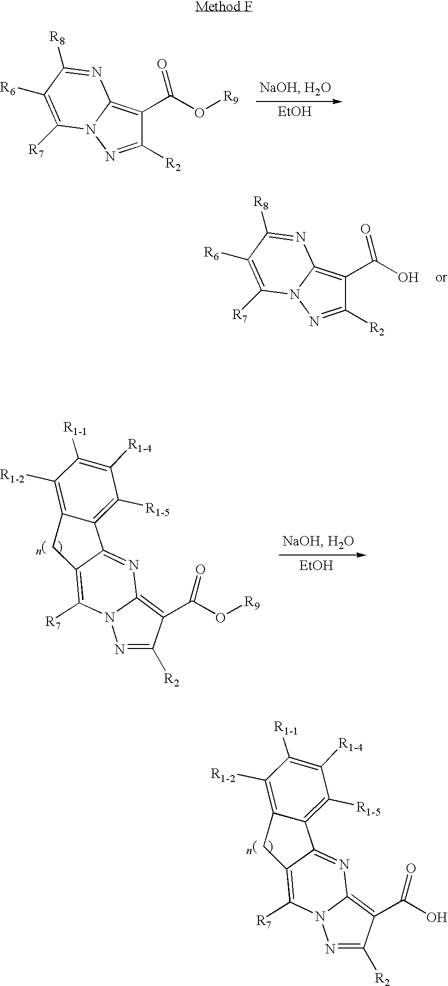

To a suspension of ester (1 equiv) in EtOH (15-20 vol) was added 1 M NaOH (1-2 equiv) and the resultant mixture stirred at a temperature range of rt to reflux until judged complete by TLC analysis. The reaction mixture was evaporated and the residue treated with water (5-10 vol) and 2 M HCl. The resulting suspension was stirred for 15 min and filtered. The solid was washed with water and dried to give the acid.

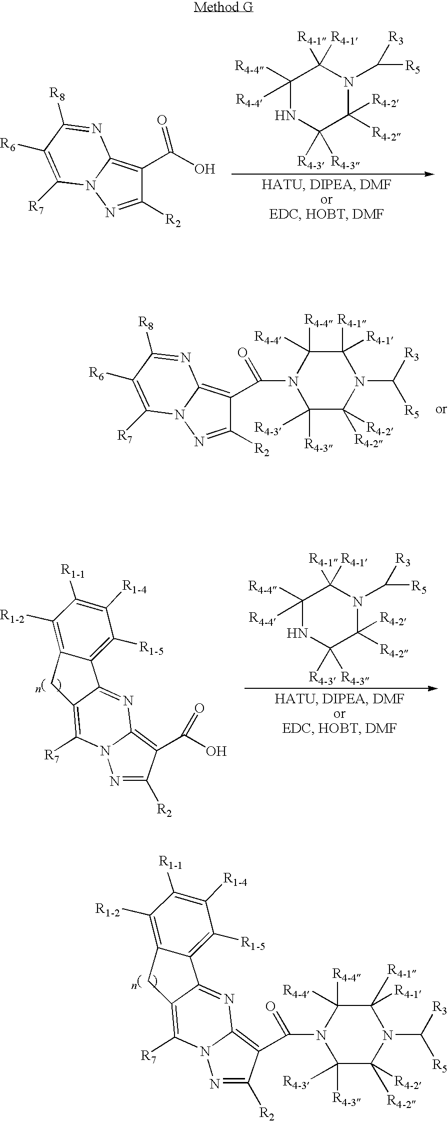

The pyrazolopyrimidine carboxylic acid (1 equiv) in DMF (50 vol) was treated with HATU (1.2 equiv) followed by DIPEA (1.2-1.4 equiv) and stirred 5-10 min at rt. This solution was transferred to a flask containing the amine (1-1.2 equiv) and the reaction stirred at rt for 16 h. Reaction progress was monitored by LC/MS. On completion the reaction mixture was diluted with water (100 vol) and extracted into EtOAc (3×50 vol). The combined organic phases were washed with water (2×50 vol) and brine (50 vol), dried (Na₂SO₄), filtered and evaporated to give the desired product. Further purification by column chromatography (silica gel, EtOAc in heptanes or MeOH in DCM) or reverse phase preparative HPLC (5-95% MeCN in water) was performed where necessary.

### Alternative Procedure:

The pyrazolopyrimidine carboxylic acid (1-1.2 equiv) in DMF (50 vol) was treated with EDC (1.1-1.2 equiv) and HOBT (1.1-1.2 equiv) and stirred 1 h at rt. This solution was transferred to a flask containing the amine (1 equiv) and the reaction stirred at rt for 16 h. Reaction progress was monitored by LC/MS. On completion the reaction mixture was diluted with satd NaHCO₃ (100 vol) and extracted into EtOAc (3×50 vol). The combined organic phases were washed with water (2×50 vol) and brine (50 vol), dried (Na₂SO₄), filtered and evaporated. Column chromatography (silica gel, EtOAc in heptanes or MeOH in DCM) or reverse phase preparative HPLC (5-95% MeCN in water) afforded the desired product.

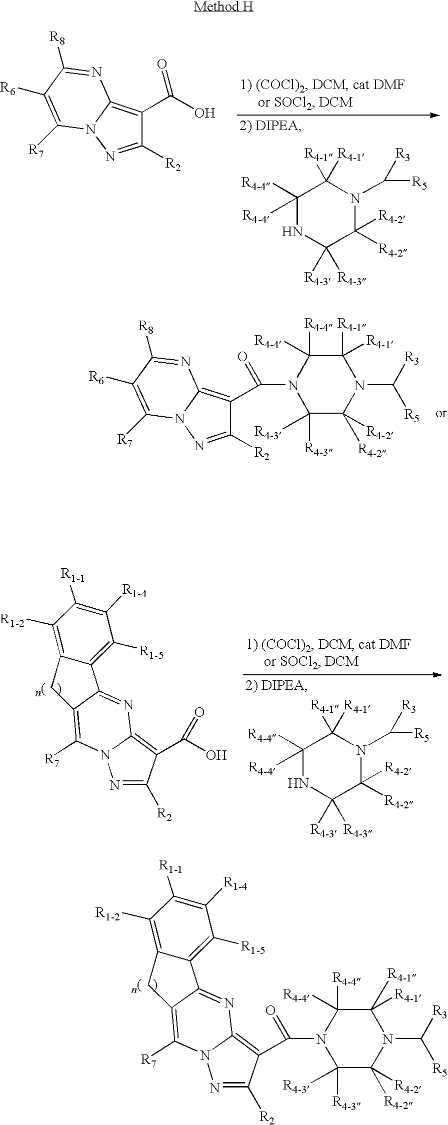

A stirred suspension of the pyrazolopyrimidine carboxylic acid in DCM (100 vol) at rt was treated with oxalyl chloride (3 equiv) followed by a few drops of DMF and the reaction mixture stirred for 0.5-1 h. The DCM and oxalyl chloride were evaporated and the residue redissolved in DCM (60 vol). A solution of the appropriate piperazine (1 equiv) and DIPEA or TEA (2 equiv) in DCM (40 vol) was added to the cooled (0° C.) reaction mixture over 5-10 min, after which time it was warmed to rt. Reaction progress was monitored by LC/MS. On completion the DCM was evaporated and the residue partitioned between satd NaHCO₃ (50 vol) and EtOAc (50 vol). The phases were separated and the aqueous phase washed with further EtOAc (2×25 vol). The combined organic phases were dried (Na₂SO₄), filtered and evaporated providing the desired product. Further purification by column chromatography (silica gel, EtOAc in heptanes or MeOH in DCM) was performed where necessary.

### Alternative Procedure:

A stirred suspension of the pyrazolopyrimidine carboxylic acid in DCM (100 vol) was treated with thionyl chloride (4-14 equiv) and the reaction mixture stirred at reflux for 0.5-1 h. The DCM and thionyl chloride were evaporated and the residue redissolved in DCM (60 vol). A solution of the appropriate piperazine (1-1.4 equiv) and DIPEA (1.1-5.7 equiv) in DCM (40 vol) was added to the cooled (0° C.) reaction mixture over 5-10 min, after which time it was warmed to rt. Reaction progress was monitored by LC/MS. On completion the DCM was evaporated and the residue partitioned between satd NaHCO₃ (50 vol) and EtOAc (50 vol). The phases were separated and the aqueous phase washed with further EtOAc (2×25 vol). The combined organic phases were dried (Na₂SO₄), filtered and evaporated providing the desired product. Further purification by column chromatography (silica gel, EtOAc in heptanes or MeOH in DCM) was performed where necessary.

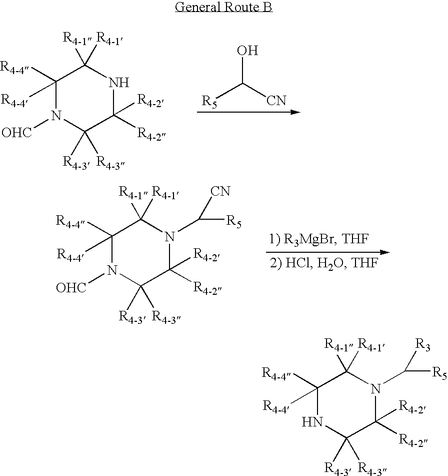

### Description of Methods Used in General Route B

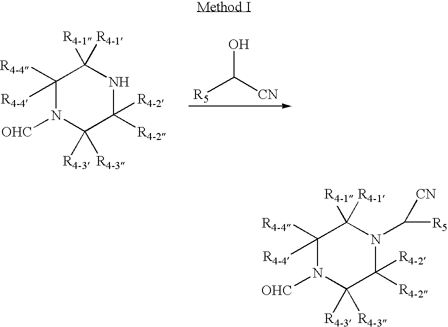

The mandelonitrile (1 equiv) and formylpiperazine (1 equiv) were stirred at 70° C. Reaction progress was monitored by LC/MS. On completion the reaction mixture was allowed to cool to rt, diluted with DCM (5 vol) and filtered. The filtrate was reduced in vacuo and purified by column chromatography (silica gel, 50-100% EtOAc in heptanes) to afford the desired product.

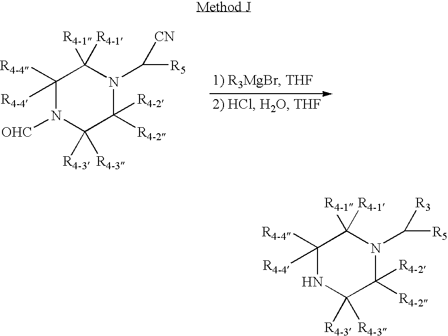

A stirring solution of the Grignard reagent (3-5 equiv) in THF (20 vol) at rt was treated with a solution of the aminonitrile (1 equiv) in THF (5 vol) and warmed at reflux for 4 h. Reaction progress was monitored by LC/MS. On completion the solvent was evaporated and the residue treated with 1 M HCl before washing with MTBE (2×10 vol). The pH of the aqueous phase was adjusted to pH 10-11 by the addition of 4 M NaOH and extracted with MTBE (3×20 vol). The combined MTBE phases were dried (Na₂SO₄), filtered and reduced in vacuo to approximately 20 vol. Excess citric acid, dissolved in MTBE (20 vol), was added to the solution of the product in MTBE, producing a cream precipitate which was isolated by filtration and washed with MTBE (3×20 vol). The solid thus obtained was treated with aqueous NaOH (20 vol, 1 M) and the aqueous extracted with DCM (3×10 vol). The combined DCM phases were dried (Na₂SO₄), filtered and reduced in vacuo to afford the desired product.

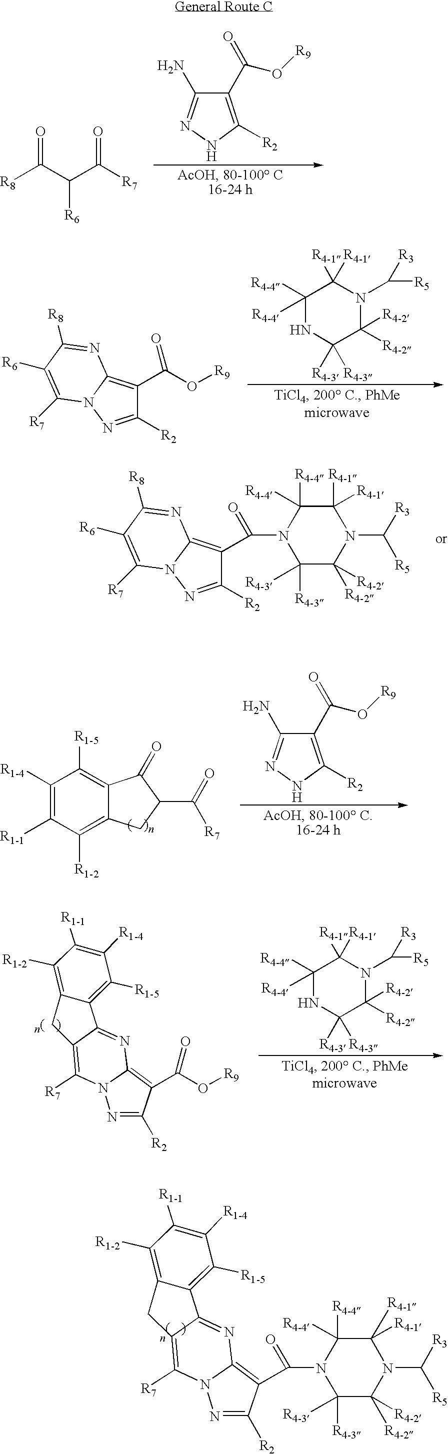

### Description of Methods Used in General Route C

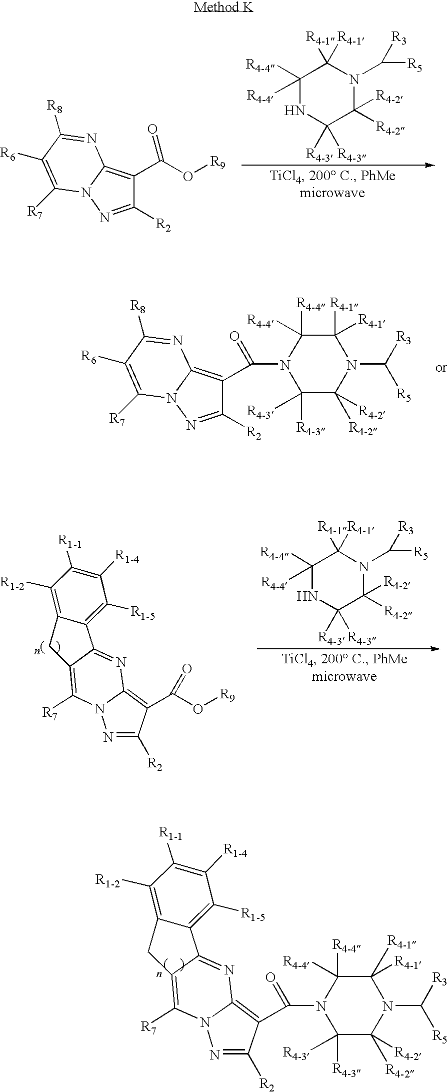

The pyrazolopyrimidine ester (1 equiv) in PhMe (50 vol) was treated with the appropriate piperazine (1-2 equiv) followed by TiCl₄ (5 equiv) then warmed to 200° C. in a sealed tube using a microwave reactor (300 W) for 10-30 min. Reaction progress was monitored by LC/MS. The reaction was diluted with satd NaHCO₃ (250 vol) and extracted into EtOAc (3×250 vol). The combined organic phases were dried (Na₂SO₄), filtered and evaporated to give a residue that was purified by column chromatography (silica gel, 40-70% EtOAc in heptanes) affording the desired product.

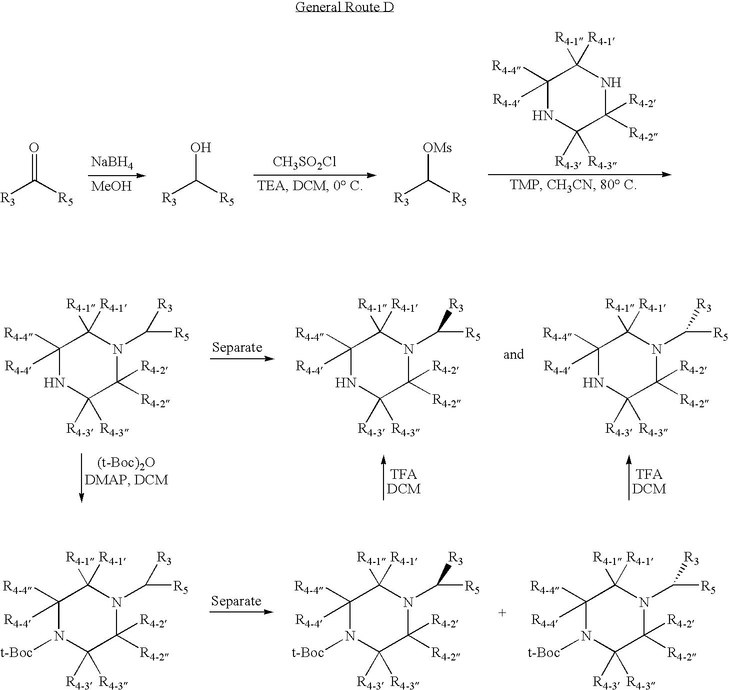

### Description of Methods Used in General Route D

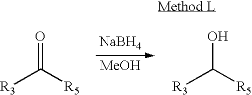

A solution of the ketone (1 equiv) in MeOH (25 vol) was treated with sodium borohydride (1.5 equiv) and stirred at rt for 15-30 min. Reaction progress was monitored by TLC. On completion the reaction was diluted with water (75 vol) and acidified to pH 1-2 by the addition of 2 M HCl. The resultant acidic aqueous was extracted with DCM (3×75 vol), the organic phases combined and dried (MgSO₄). Filtration followed by reduction of the filtrate in vacuo afforded the desired product.

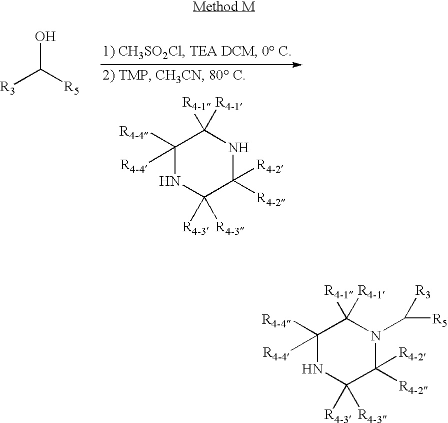

A solution of the alcohol (1 equiv) and TEA (2 equiv) in DCM (10 vol) at 0° C. was treated with MsCl (1.3 equiv) and stirred at this temperature for 1 h. Reaction progress was monitored by TLC. On completion the reaction mixture was washed with ice-cold water (2×10 vol), 1 M HCl (10 vol) and satd NaHCO₃ (10 vol). The DCM phase was dried (MgSO₄) and filtered and the filtrate reduced in vacuo in the absence of a warming water bath. The mesylate thus formed was immediately dissolved in MeCN (10 vol), treated with the appropriate piperazine (0.8 equiv) and 2,2,6,6-tetramethylpiperidine (0.8 equiv) and heated at reflux for 16 h. Reaction progress was monitored by LC/MS. On completion the reaction mixture was allowed to cool to rt, diluted with DCM (50 vol) and washed with water (25 vol) and brine (25 vol). The DCM phase was dried (MgSO₄) and filtered and the filtrate reduced in vacuo to obtain the desired product.

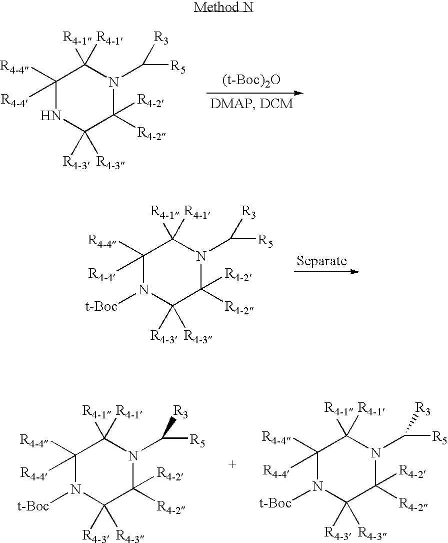

A solution of the diastereomeric mixture of piperazines (1 equiv) in DCM (25 vol) was treated with di-tert-butyl dicarbonate (1.1 equiv) followed by DMAP (1 molar %) and stirred at rt for 16 h. Reaction progress was monitored by LC/MS. On completion the reaction was diluted with water (50 vol) and DCM (25 vol) and the organic phase isolated and washed with 1 M HCl (50 mL) and water (50 mL). The DCM phase was dried (MgSO₄) and filtered and the filtrate reduced in vacuo. Column chromatography (silica gel, 2-5% EtOAc in heptanes) separated the desired diastereomers.

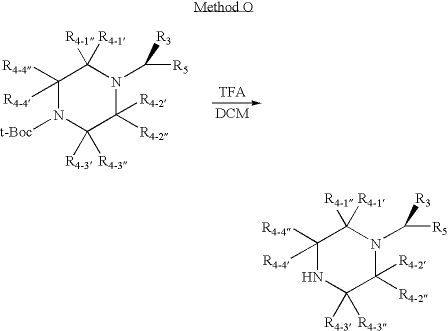

The t-Boc carbamate (1 equiv) in DCM (15 vol) was treated with TFA (5 vol) and stirred at rt for 1-5 h. Reaction progress was monitored by LC/MS. On completion, the reaction solvent was evaporated and the residue obtained carefully treated with satd NaHCO₃ solution (50 vol). The aqueous was extracted with DCM (3×30 vol), the combined organic phases dried (Na₂SO₄) and filtered and the filtrate reduced in vacuo to afford the desired product.

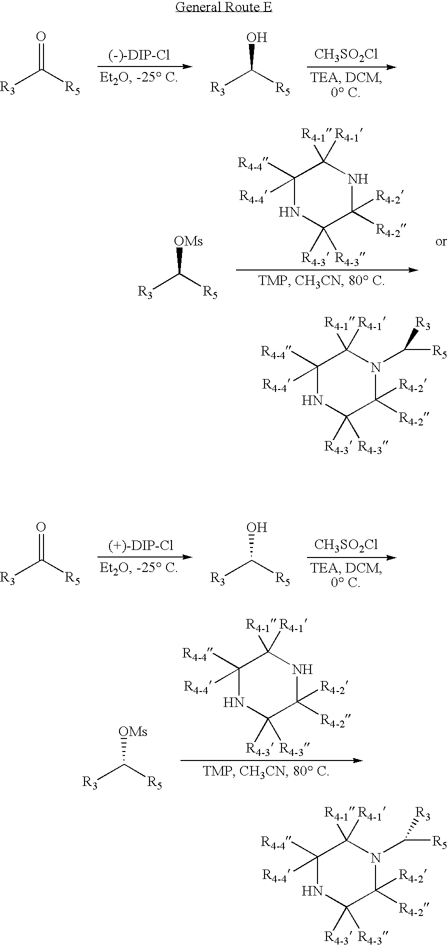

### Description of Methods Used in General Route E

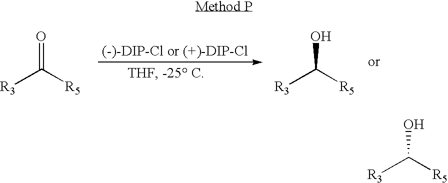

In accordance with the procedure of Brown et al. (J. Am. Chem. Soc., 1988, 110, 1539), a stirred solution of (+) or (−)-DIP-Cl (1.4-3.3 equiv) in THF (20 vol) was cooled to −25 to −30° C. and treated with the ketone (1 equiv). The reaction was maintained at this temperature for 1-16 h before treating with acetaldehyde (0.4-2.3 equiv). After warming to rt over 0.5-1 h, the solvent was evaporated and the residue obtained redissolved in Et₂O (20 vol). Diethanolamine (1.4-3.3 equiv) was added and the reaction mixture stirred 1-2 h or until a thick white precipitate had formed, at which juncture it was filtered and the filtrate evaporated. Column chromatography (silica gel, EtOAc in heptanes) afforded the desired product.

### Alternative Procedure:

In accordance with the procedure of Brown et al. (J. Am. Chem. Soc., 1988, 110, 1539), a stirred solution of (+) or (−)-DIP-Cl (2.2 equiv) in THF (20 vol) was cooled to −25 to −30° C. and treated with the ketone (1 equiv). The reaction was maintained at this temperature for 1-16 h before treating with acetaldehyde (1.2 equiv). After warming to rt over 0.5-1 h, the solvent was evaporated and the residue partitioned between water (10 vol) and MTBE (20 vol). The phases were separated and the aqueous phase washed with MTBE (20 vol) before basifying to pH 8-9 by careful addition of solid NaHCO₃. The basic aqueous was extracted with MTBE (3×20 vol), the MTBE phases combined, dried (Na₂SO₄), filtered and reduced in vacuo. Column chromatography (silica gel, EtOAc in heptanes) afforded the desired product.

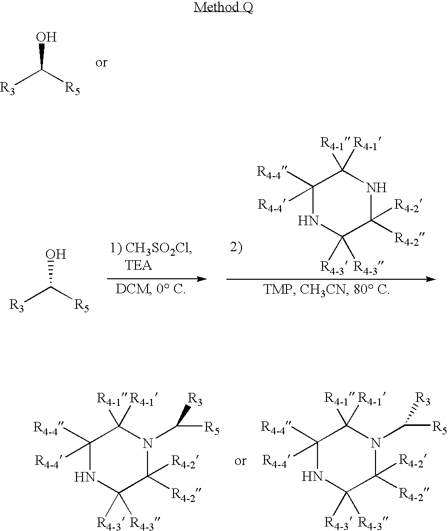

In accordance with the procedure of Taget et al. (J. Med. Chem., 2001, 44, 3343-3346), a solution of the alcohol (1 equiv) and TEA (2 equiv) in DCM (10 vol) at 0° C. was treated with MsCl (1.3 equiv) and stirred at this temperature for 1 h. Reaction progress was monitored by TLC. On completion the reaction mixture was washed with ice-cold water (2×10 vol), 1 M HCl (10 vol) and satd NaHCO₃ (10 vol). The DCM phase was dried (MgSO₄) and filtered and the filtrate reduced in vacuo in the absence of a warming water bath. The mesylate thus formed was immediately dissolved in MeCN (10 vol), treated with the appropriate piperazine (1 equiv) and 2,2,6,6-tetramethylpiperidine (1.03 equiv) and heated at reflux for 16 h. Reaction progress was monitored by LC/MS. On completion the reaction mixture was allowed to cool to rt, diluted with DCM (50 vol), washed with water (25 vol) and extracted with 2 M HCl (25 vol). The acidic aqueous phase was washed with DCM (2×25 vol), raised to pH 14 by the addition of 4 M NaOH, and extracted into DCM (3×25 vol). The combined DCM phases were dried (MgSO₄) and filtered and the filtrate reduced in vacuo. Where necessary, column chromatography (silica gel, 0-5% MeOH in DCM with 1% TEA or 0-10% MeOH in EtOAc with 1% TEA) was employed to obtain the desired product.

### First Alternative Procedure:

A solution of the alcohol (1 equiv) and TEA (2 equiv) in DCM (10 vol) at 0° C. was treated with MsCl (1.3 equiv) and stirred at this temperature for 1 h. Reaction progress was monitored by TLC. On completion the reaction mixture was washed with ice-cold satd NaHCO₃ (10 vol) and water (10 vol). The DCM phase was dried (Na₂SO₄) and filtered and the filtrate reduced in vacuo in the absence of a warming water bath. The mesylate thus formed was immediately dissolved in cold (0° C.) MeCN (10 vol), treated with the appropriate piperazine (1 equiv) and TEA (1.03 equiv) and stirred for 16-64 h, during which time it was allowed to warm to rt. Reaction progress was monitored by LC/MS. On completion the reaction mixture was diluted with DCM (50 vol), washed with water (25 vol) and extracted with 2 M HCl (25 vol). The acidic aqueous phase was washed with DCM (2×25 vol), the pH raised to pH 14 by the addition of 4 M NaOH and extracted into DCM (3×25 vol). The combined DCM phases were dried (MgSO₄) and filtered and the filtrate reduced in vacuo. Where necessary, column chromatography (silica gel, 0-5% MeOH in DCM with 1% TEA or 0-10% MeOH in EtOAc with 1% TEA) was employed to obtain the desired product.

### Second Alternative Procedure:

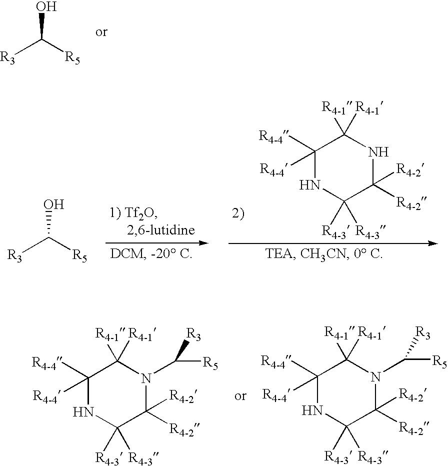

A stirred solution of the alcohol (1 equiv) in DCM (20 vol) at −20° C. was treated with 2,6-lutidine (1.9 equiv) followed by a solution of trifluoromethanesulfonic anhydride (1.75 equiv) in DCM (10 vol), added over 5-10 min. After stirring a further 45 min at this temperature, the reaction was warmed to 0° C. before washing with ice-cold satd NaHCO₃ (15 vol) and water (15 vol). The DCM phase was dried (Na₂SO₄) and filtered and the filtrate reduced in vacuo in the absence of a warming water bath. The triflate thus formed was immediately dissolved in cold (0° C.) MeCN (5 vol) and added to a stirred solution of the appropriate piperazine (1.4 equiv) and TEA (1.5 equiv) in MeCN (15 vol), also at 0° C. The reaction mixture was allowed to warm to rt and stirred 16 h; reaction progress was monitored by LC/MS. On completion the reaction was diluted with DCM (50 vol) and washed with water (25 vol) and satd NaHCO₃ (25 vol). The DCM phase was dried (MgSO₄) and filtered and the filtrate reduced in vacuo to obtain the desired product.

### Examples of Compounds Prepared Employing General Routes A, B, C, D, and E

### Example 1

### 4,4,4-Trifluoro-1-(4-methoxyphenyl)-1,3-butanedione (1)

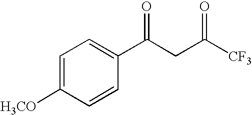

Potassium tert-butoxide (2.24 g, 20.0 mmol) was added to a solution of 4′-methoxyacetophenone (2.50 g, 16.7 mmol) in toluene (50 mL) at 0° C. and the resulting solution stirred for 5-10 min. Ethyl trifluoroacetate (2.38 mL, 20.0 mmol) was added dropwise over 5-10 min and the reaction mixture stirred for 16 h at rt. The reaction mixture was filtered and the residue washed with MTBE (15 mL) then dried in air. The residue was suspended in 3 M HCl (25 mL), stirred for 5-10 min and extracted into EtOAc (3×25 mL). The combined organic phases were dried (Na₂SO₄), filtered and evaporated to give the title compound as a cream colored solid: LC/MS t_{R} 1.79 min; MS (ES+) m/z 247, 265.

### Example 2

### 4,4,4-Trifluoro-1-(4-methoxyphenyl)-2-methyl-1,3-butanedione (2)

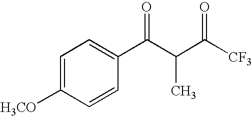

4′-Methoxypropiophenone (500 mg, 3.04 mmol) was treated with LHMDS (6.09 mL, 6.09 mmol, 1 M solution in THF) and 1-(trifluoroacetyl)imidazole (1.38 mL, 12.1 mmol, d 1.441) using Method C to give the title compound as a pale yellow oil: ¹H NMR δ_{H} (250 MHz, CDCl₃) 7.97 (2H, d), 7.02 (2H, d), 4.90 (1H, q), 3.93 (3H, s), 1.56 (3H, d).

### Example 3

### 2-Fluoro-1-(4-methoxyphenyl)ethanone (3)

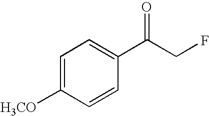

A solution of 2-bromo-4′-methoxyacetophenone (2.50 g, 10.9 mmol) and cesium fluoride (8.29 g, 54.5 mmol) in 1-n-butyl-3-methylimidazolium hexafluorophosphate (14 mL), water (0.19 mL, 10.91 mmol) and MeCN (28 mL) was heated to 100° C. for 4 h. The reaction was cooled to rt and water (100 mL) was added followed by MTBE (100 mL) to give a three-phase system. The aqueous layer was separated and organic and ionic phases jointly washed with water (3×10 mL). The organic phase was separated and the ionic liquid extracted with MTBE (3×100 mL). The combined MTBE layers were washed with brine, dried (Na₂SO₄), filtered and evaporated. The orange oil was purified by column chromatography (silica gel, 5% EtOAc in heptanes) to give the title compound as a yellow oil: LC/MS t_{R} 1.08 min; MS (ES+) m/z 169.

### Example 4

### 2,4,4,4-Tetrafluoro-1-(4-methoxyphenyl)-1,3-butanedione (4)

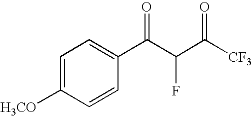

Ketone 3 (2.37 g, 14.1 mmol) was treated with LHMDS (16.9 mL, 16.9 mmol, 1 M solution in THF) and 1-(trifluoroacetyl)imidazole (3.94 g, 2.68 mmol) using Method C. The residue obtained was purified by column chromatography (silica gel, 10% EtOAc in heptanes) to give the title compound as a yellow oil: ¹H NMR δ_{H} (360 MHz, CDCl₃) 7.98 (2H, d), 6.93 (2H, d), 5.61 (1H, d), 3.84 (3H, s).

### Example 5

### 3,4-Dihydro-6-methoxy-2-(trifluoroacetyl)-1(2H)-naphthalenone (5)

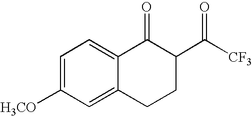

A solution of 6-methoxy-1-tetralone (5 g, 28.3 mmol) in toluene (110 mL) was cooled to 0° C. and treated with KOt-Bu (3.82 g, 34.0 mmol) in a single portion. The reaction was stirred at rt for 30 min and cooled back down to 0° C. Ethyl trifluoroacetate (4.05 mL, 34.0 mmol, d 1.194) was added dropwise over 10 min and the reaction stirred at rt for 22 h. The mixture was diluted with EtOAc (100 mL) and washed with aqueous HCl (100 mL, 1.2 M) and brine (100 mL). The organic layer was dried (MgSO₄), filtered and evaporated to a residue which was purified by column chromatography (silica gel, 50% EtOAc in heptanes) to give the title compound as a yellow-orange solid: LC/MS t_{R} 1.95 min; MS (ES+) m/z 273.

### Example 6

### 1-(4-Cyclopropylphenyl)-1-propanone (6)

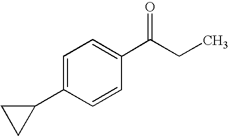

Cyclopropylbenzene (500 mg, 4.2 mmol) was treated with propionyl chloride (0.36 mL, 4.2 mmol, d 1.065), and AlCl₃ (559 mg, 4.2 mmol) using Method B to give the title compound as a pale yellow oil: LC/MS t_{R} 2.11 min; MS (ES+) m/z 175.

### Example 7

### 1-(4-Cyclopropylphenyl)-4,4,4-trifluoro-2-methyl-1,3-butanedione (7)

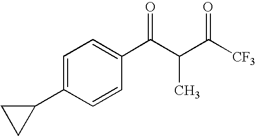

Ethyl ketone 6 (114 mg, 0.66 mmol) was treated with LHMDS (0.8 mL 0.8 mmol, 1 M solution in THF) and 1-(trifluoroacetyl)imidazole (0.3 mL, 2.64 mmol, d 1.441) using Method C to give the title compound as a pale yellow oil: ¹H NMR δ_{H} (250 MHz, CDCl₃) 7.77 (2H, d), 7.10 (2H, d), 4.80 (1H, q), 1.89 (1H, m), 1.45 (3H, d), 1.04 (2H, m), 0.78 (2H, m).

### Example 8

### 1-(4-Ethylphenyl)-1-propanone (8)

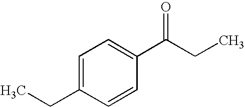

Ethylbenzene (500 mg, 4.72 mmol) was treated with propionyl chloride (0.4 mL, 4.72 mmol, d 1.065), and AlCl₃ (627 mg, 4.72 mmol) using Method B to give the title compound as a pale yellow oil: ¹H NMR δ_{H} (250 MHz, CDCl₃) 7.80 (2H, d), 7.18 (2H, d), 2.89 (2H, q), 2.61 (2H, q), 1.16 (3H, t), 1.12 (3H, t).

### Example 9

### 1-(4-Ethylphenyl)-4,4,4-trifluoro-2-methyl-1,3-butanedione (9)

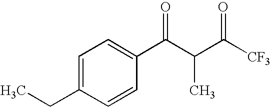

Ethyl ketone 8 (200 mg, 1.23 mmol) was treated with LHMDS (1.4 mL, 1.4 mmol, 1 M solution in THF) and 1-(trifluoroacetyl)imidazole (0.56 mL, 4.94 mmol, d 1.441) using Method C to give the title compound as a pale yellow oil: ¹H NMR δ_{H} (250 MHz, CDCl₃) 7.89 (2H, d), 7.36 (2H, d), 4.91 (1H, q), 2.75 (2H, q), 1.54 (3H, d), 1.29 (3H, t).

### Example 10

### 2-Bromo-1-[4-(1-methylethyl)phenyl]ethanone (10)

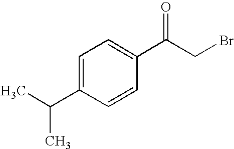

Isopropylbenzene (500 mg, 4.17 mmol) was treated with bromoacetyl bromide (0.36 mL, 4.17 mmol d 2.31), and AlCl₃ (554 mg, 4.17 mmol) using Method B. The reaction was poured onto ice (15 mL) and extracted with EtOAc (3×15 mL). The combined organic layers were dried (Na₂SO₄), filtered and evaporated to give a residue which was purified by column chromatography (silica gel, 10% EtOAc in heptanes) providing the title compound as a brown oil: LC/MS t_{R} 2.27 min; MS (ES+) m/z 241, 243.

### Example 11

### 2-Fluoro-1-[4-(1-methylethyl)phenyl]ethanone (11)

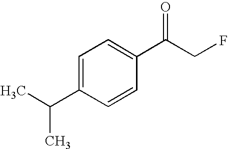

A solution of ketone 10 (699 mg, 2.9 mmol) and potassium fluoride (841 mg, 14.5 mmol) in 1-n-butyl-3-methylimidazolium hexafluorophosphate (5 mL), water (0.26 mL, 14.5 mmol) and MeCN (10 mL) was heated to 100° C. for 4 h. A further 2 equiv of KF (336 mg, 5.8 mmol) was added the reaction heated for a further 2 h. The reaction was cooled to rt and water (20 mL) was added followed by MTBE (20 mL) to give a three-phase system. The aqueous layer was separated and organic and ionic phases jointly washed with water (3×25 mL). The organic phase was separated and the ionic liquid extracted with MTBE (3×10 mL). The combined MTBE layers were washed with brine (1×10 mL), dried (Na₂SO₄), filtered and evaporated. The orange oil obtained was purified by column chromatography (silica gel, 5% EtOAc in heptanes) to give the title compound as a yellow oil: ¹H NMR δ_{H} (250 MHz, CDCl₃) 7.84 (2H, d) 7.36 (2H, d) 5.52 (2H, d) 2.99 (1H, m) 1.28 (6H, d).

### Example 12

### 2,4,4,4-Tetrafluoro-1-[4-(1-methylethyl)phenyl]-1,3-butanedione (12)

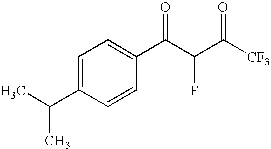

Ketone 11 (242 mg, 1.34 mmol) was treated with LHMDS (1.8 mL, 1.8 mmol, 1 M solution in THF) and 1-(trifluoroacetyl)imidazole (0.61 mL, 5.36 mmol, d 1.441) using Method C. The residue obtained was purified by column chromatography (silica gel, 5% EtOAc in heptanes) to give the title compound as a yellow oil: ¹H NMR δ_{H} (360 MHz, CDCl₃) 8.02 (2H, d), 7.43 (2H, d), 5.74 (1H, d), 3.04 (1H, m), 1.32 (6H, d).

### Example 13

### 2-Cyano-3-ethoxy-2-propenoic Acid, Methyl Ester (13)

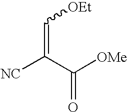

A mixture of methyl cyanoacetate (52 mL, 0.59 mol), TEOF (98 mL, 0.59 mol) and Ac₂O (120 mL, 1.27 mol) was heated to 165° C. for 3 h. The volatiles were removed by distillation at 110° C. and 2 mbar to leave the title compound.

### Example 14

### 3-Amino-1H-pyrazole-4-carboxylic Acid, Methyl Ester (14)

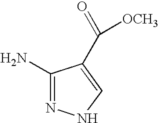

To a solution of 13 (61.5 g, 0.39 mol) in MeOH (600 mL) was added hydrazine hydrate (19.8 g, 0.39 mol). The resulting suspension was heated to 90° C. for 18 h after which time the resulting yellow solution was evaporated. The residue was washed with MeOH (100 mL), filtered and dried to give the title compound. The filtrate was evaporated to a small volume and MeOH (30 mL) was added. The resulting precipitate was filtered and dried to give a further crop of the title compound: LC/MS t_{R} 0.18 min; MS (ES+) m/z 141.

### Example 15

### 5-(4-Methoxyphenyl)-7-(trifluoromethyl)pyrazolo[1,5-a]pyrimidine-3-carboxylic Acid, Methyl Ester (15)

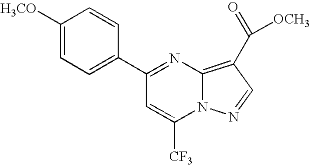

Aminopyrazole 14 (1.41 g, 0.01 mol) was treated with diketone 1 (2.75 g, 0.01 mol) using Method D to give the title compound as pale yellow solid: LC/MS t_{R} 1.54 min; MS (ES+) m/z 352, 725.

### Example 16

### 5-(4-Methoxyphenyl)-7-(trifluoromethyl)pyrazolo[1,5-a]pyrimidine-3-carboxylic Acid (16)

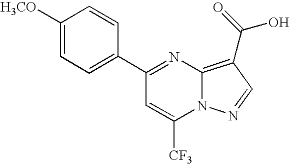

Methyl ester 15 (175 mg, 0.50 mmol) was treated with 1 M NaOH in accordance with Method F to give the title compound as a white powder: LC/MS t_{R} 1.30 min; MS (ES+) m/z 338.

### Example 17

### 3-Amino-1H-pyrazole-4-carboxylic Acid, 3-Propenyl Ester (17)

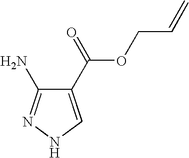

3-Amino-1H-pyrazole-4-carboxylic acid (5.0 g, 39.4 mmol) was treated with cesium carbonate (15.4 g, 47.2 mmol) and allyl bromide (5.6 mL, 43.3 mmol, d 1.398) using Method A to give the title compound as a viscous caramel colored oil: LC/MS t_{R} 1.15 min; MS (ES+) m/z 168; ¹H NMR δ_{H} (250 MHz, CDCl₃) 7.73 (1H, s), 6.25 (2H, br s) 5.90-6.06 (1H, m), 5.35 (1H, d), 5.25 (1H, d), 4.72 (2H, d).

### Example 18

### Alternative Method for the Preparation of 3-Amino-1H-pyrazole-4-carboxylic Acid, 3-Propenyl Ester (17)

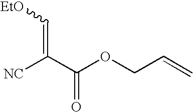

A mixture of allyl cyanoacetate (4.84 g, 38.7 mmol, d 1.065), TEOF (6.4 mL, 38.7 mmol, d 0.891) and Ac₂O (9.2 mL, 83.6 mmol, d 1.08) was heated at 165° C. for 3.5 h whilst removing the forming EtOAc by distillation. After cooling to rt, the mixture was reduced in vacuo. Removal of the volatiles by distillation at 95-100° C. and 2 mbar provided 2-cyano-3-ethoxy-2-propenoic acid 3-propenyl ester as a dark red oil: LC/MS t_{R} 1.68 min; MS (ES+) m/z 182; ¹H NMR δ_{H} (250 MHz, CDCl₃) 8.03 (1H, s), 5.95 (1H, app ddt), 5.38 (1H, app dq), 5.28 (1H, app dq), 4.71 (2H, app dt), 4.36 (2H, q), 1.45 (3H, t).

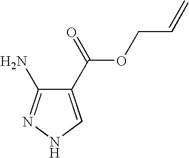

Hydrazine hydrate (1.30 mL, 27.4 mmol, d 1.030) was added to a stirred solution of 2-cyano-3-ethoxy-2-propenoic acid 3-propenyl ester (4.97 g, 27.4 mmol) in MeOH (50 mL) and the mixture heated at 90° C. for 18 h. The orange solution was allowed to cool to rt before reducing in vacuo to yield the title compound as a viscous caramel colored oil with spectral data consistent with that obtained for 17 as prepared in example 17.

### Example 19

### 5-(4-Methoxyphenyl)-6-methyl-7-(trifluoromethyl)pyrazolo[1,5-a]pyrimidine-3-carboxylic Acid, 3-Propenyl Ester (18)

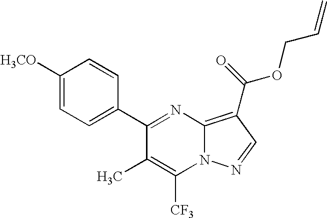

Aminopyrazole 17 (5.87 g, 35.1 mmol) was treated with diketone 2 (5.94 g, 22.8 mmol) using Method D to give the title compound as pale yellow solid: LC/MS t_{R} 2.45 min; MS (ES+) m/z 392, 805; ¹H NMR δ_{H} (250 MHz, CDCl₃) 8.61 (1H, s), 7.62 (2H, d), 7.03 (2H, d), 5.98-6.13 (1H, m), 5.50 (1H, d), 5.27 (1H, d), 4.86 (2H, d), 3.89 (3H, s), 2.59 (3H, q).

### Example 20

### 5-(4-Methoxyphenyl)-6-methyl-7-(trifluoromethyl)pyrazolo[1,5-a]pyrimidine-3-carboxylic Acid (19)

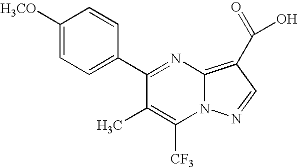

Pyrazolopyrimidine allyl ester 18 (4.38 g, 11.2 mmol) was treated with 1,3-dimethylbarbituric acid (1.75 g, 11.2 mmol) and Pd(PPh₃)₄ (1.29 g, 1.12 mmol) using Method E to give the title compound as an off-white solid: LC/MS t_{R} 1.95 min; MS (ES+) m/z 352, 725; ¹H NMR δ_{H} (250 MHz, DMSO-d₆) 12.63 (1H, br s), 8.63 (1H, s), 7.60 (2H, d), 7.11 (2H, d), 3.84 (3H, s), 2.44 (3H, q).

### Example 21

### [5-(4-Methoxyphenyl)-7-(trifluoromethyl)pyrazolo[1,5-a]pyrimidin-3-yl][4-(phenylmethyl)-1-piperazinyl]methanone (20)

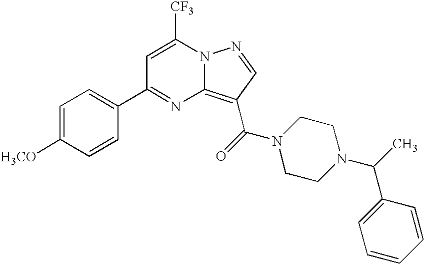

In accordance with Method H, carboxylic acid 16 (50 mg, 0.15 mmol) was converted to the analogous acid chloride via treatment with oxalyl chloride (0.13 mL, 1.50 mmol, d 1.455) and catalytic DMF (5 μL) then treated with TEA (42 μL, 0.31 mmol, d 0.742) and 1-(1-phenylethyl)piperazine (34 mg, 0.178 mmol) providing the amide 20 as a yellow solid: LC/MS t_{R} 1.91 min; MS (ES+) m/z 510; ¹H NMR δ_{H} (400 MHz, CDCl₃) 8.39 (1H, s), 8.02 (2H, d), 7.56 (1H, s), 7.12-7.27 (5H, m), 6.97 (2H, d), 3.85 (3H, s), 3.71-3.91 (2H, br s), 3.52-3.70 (2H, br s), 3.37 (1H, q), 2.27-2.69 (4H, m), 1.33 (3H, d).

Chiral column chromatography employing a Chiralpak “AD” column (5 cm×0.46 cm, 10 micron pore size, eluting with 20% EtOAc in heptanes at 1 mL/min) separated the amide into the individual enantiomers (20a) >99% ee and (20b) >99% ee.

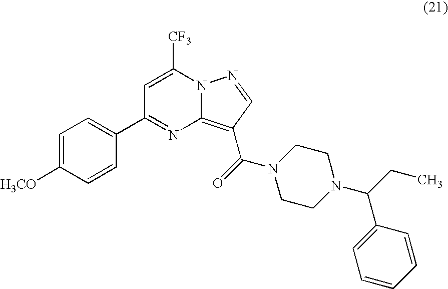

### Example 22

### 4-Formyl-α-phenyl-1-piperazineacetonitrile (22a)

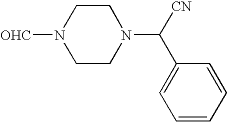

Mandelonitrile (6.65 g, 40 mmol, 80% technical grade) was treated with 1-formylpiperazine (3.42 g, 30 mmol) in accordance with Method I to afford the title compound after column chromatography (silica gel, 50% EtOAc in heptanes) as a colorless oil: ¹H NMR δ_{H} (400 MHz, CDCl₃) 8.04 (1H, s), 7.50-7.57 (2H, m), 7.37-7.48 (3H, m), 4.91 (1H, s), 3.60 (2H, br s), 3.33-3.49 (2H, m), 2.54-2.66 (4H, m).

### Example 23

### 1-(1-Phenylpropyl)piperazine (22b)

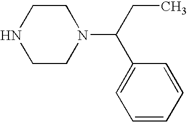

Aminonitrile 22a (0.46 g, 2.0 mmol) was treated with ethyl magnesium bromide (10 mL, 10 mmol, 1 M solution in THF) in accordance with Method J to afford the title compound as a pale yellow oil: LC/MS t_{R} 0.87 min; MS (ES+) m/z 205.

### Example 24

### [5-(4-Methoxyphenyl)-7-(trifluoromethyl)pyrazolo[1,5-a]pyrimidin-3-yl][4-(1-phenylpropyl)-1-piperazinyl]methanone (21)

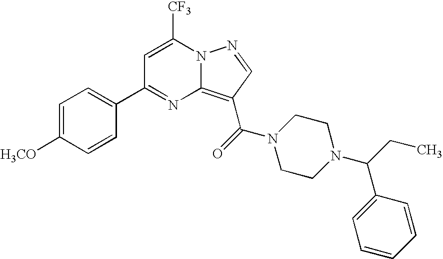

In accordance with Method H (alternative procedure) carboxylic acid 16 (168 mg, 0.50 mmol) was converted to the analogous acid chloride using thionyl chloride (0.15 mL, 2.06 mmol, d 1.631) then treated with DIPEA (0.10 mL, 0.57 mmol, d 0.742) and piperazine 22b (102 mg, 0.50 mmol). Column chromatography (silica gel, 40-60% EtOAc in cyclohexane) afforded the title compound as a vitreous yellow solid: LC/MS t_{R} 1.61 min; MS (ES+) m/z 524; ¹H NMR δ_{H} (400 MHz, CDCl₃) 8.45 (1H, s), 8.09 (2H, d), 7.64 (1H, s), 7.19-7.39 (5H, m), 7.05 (2H, d), 3.94 (3H, s), 3.86 (2H, br s), 3.68 (2H, br s), 3.26 (1H, dd), 2.59 (2H, br s), 2.47 (2H, br s), 1.97 (1H, m), 1.77 (1H, m), 0.77 (3H, t).

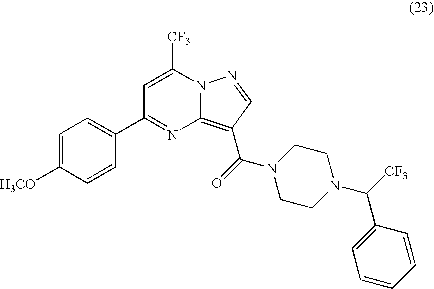

### Example 25

### 1-(2,2,2-Trifluoro-1-phenylethyl)piperazine (24)

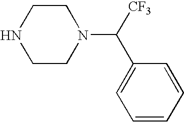

A solution of 2,2,2-trifluoroacetophenone (1.40 g, 8.04 mmol) and piperazine (0.71 g, 8.24 mmol) in DCM (50 mL) was treated with TiCl₄ (4.10 mL, 4.10 mmol, 1 M solution in DCM) and stirred at rt for 16 h. A solution of sodium cyanoborohydride (1.50 g, 23.9 mmol) in MeOH (20 mL) was added and the reaction stirred a further 30 min before diluting with 5 M NaOH (30 mL) and EtOAc (100 mL). The organic phase was isolated and the aqueous washed with a further portion of EtOAc (50 mL). The organic phases were combined, dried (MgSO₄) and filtered and the filtrate reduced in vacuo. Reverse phase preparative HPLC (0.1% TFA, 5-95% MeCN in water) afforded the TFA salt of the title compound as a white solid: LC/MS t_{R} 1.32 min; MS (ES+) m/z 245.

### Example 26

### [5-(4-Methoxyphenyl)-7-(trifluoromethyl)pyrazolo[1,5-a]pyrimidin-3-yl][4-(2,2,2-trifluoro-1-phenylethyl)-1-piperazinyl]methanone (23)

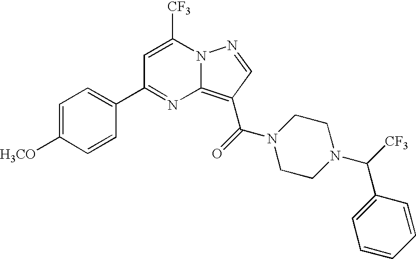

In accordance with Method H (alternative procedure), carboxylic acid 16 (68 mg, 0.20 mmol) was converted to the analogous acid chloride using thionyl chloride (0.20 mL, 2.74 mmol, d 1.631) then treated with DIPEA (0.20 mL, 1.14 mmol, d 0.742) and the TFA salt of piperazine 24 (135 mg, 0.29 mmol). Column chromatography (silica gel, 30-40% EtOAc in heptanes) afforded the title compound as a vitreous yellow solid: LC/MS t_{R} 1.95 min; MS (ES+) m/z 564; ¹H NMR δ_{H} (400 MHz, CDCl₃) 8.47 (1H, s), 8.09 (2H, d), 7.65 (1H, s), 7.40 (5H, br s), 7.06 (2H, d), 4.14 (1H, q), 3.94 (3H, s), 3.88 (2H, br s), 3.73 (2H, br s), 2.58-2.96 (4H, br m).

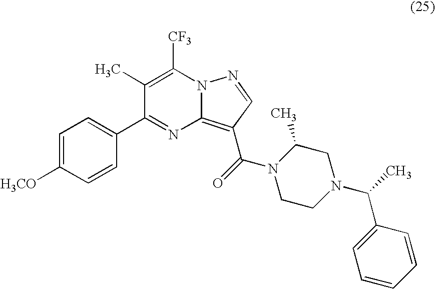

### Example 27

### (3R)-3-Methyl-1-[(1R)-1-phenylethyl]piperazine (26)

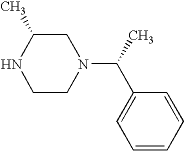

In accordance with Method Q (first alternative procedure), (S)-(−)-1-phenylethanol (250 mg, 2.05 mmol) was treated with MsCl (0.21 mL, 2.66 mmol, d 1.48) and TEA (0.57 mL, 4.10 mmol, d 0.726) to fashion the corresponding mesylate. (R)-(−)-2-Methylpiperazine (285 mg, 2.86 mmol) was treated immediately with this mesylate and TEA (0.43 mL, 3.07 mmol, d 0.726). Column chromatography (silica gel, 4% MeOH in EtOAc with 1% TEA) afforded the title compound as a pale yellow oil: LC/MS t_{R} 0.46 min; MS (ES+) m/z 205; ¹H NMR δ_{H} (360 MHz, CDCl₃) 7.21-7.38 (5H, m), 6.16 (1H, br s), 3.43 (1H, q), 3.29 (1H, app dt), 2.98-3.14 (3H, m), 2.72 (1H, app dt), 2.33 (1H, app td), 1.94 (1H, dd), 1.37 (3H, d), 1.18 (3H, d).

### Example 28

### [5-(4-Methoxyphenyl)-6-methyl-7-(trifluoromethyl)pyrazolo[1,5-a]pyrimidin-3-yl][(2R)-2-methyl-4-[(1R)-1-phenylethyl]-1-piperazinyl]methanone (25)

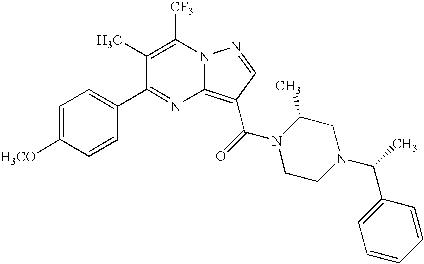

Carboxylic acid 19 (27 mg, 0.078 mmol) was treated with HATU (35 mg, 0.093 mmol), DIPEA (16 μL, 0.093 mmol, d 0.742) and piperazine 26 (19 mg, 0.093 mmol) in accordance with Method G to afford the title compound after column chromatography (silica gel, 30-50% EtOAc in heptanes) as a vitreous yellow solid: LC/MS t_{R} 1.72 min; MS (ES+) m/z 538; ¹H NMR δ_{H} (360 MHz, CDCl₃) 8.44 (1H, s), 7.53 (2H, d), 7.20-7.43 (5H, m), 7.05 (2H, d), 3.58-5.15 (2H, br m), 3.95 (3H, s), 3.41 (1H, q), 3.34 (1H, br s), 2.82 (1H, br s), 2.69 (1H, br s), 2.56 (3H, q), 2.35 (1H, dd), 2.14 (1H, app td), 1.43 (3H, d), 1.34 (3H, d).

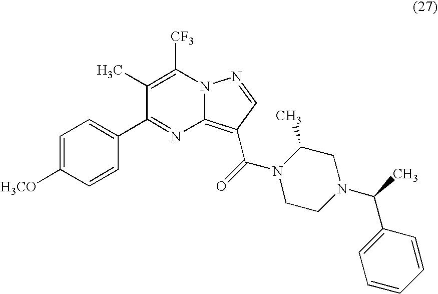

### Example 29

### (3S)-3-Methyl-1-[(1R)-1-phenylethyl]piperazine (28)

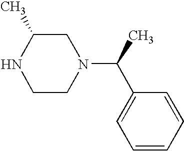

In accordance with Method Q (first alternative procedure), (R)-(−)-1-phenylethanol (144 mg, 1.18 mmol) was treated with MsCl (0.12 mL, 1.54 mmol, d 1.48) and TEA (0.34 mL, 2.46 mmol, d 0.726) to fashion the corresponding mesylate. (R)-(−)-2-Methylpiperazine (232 mg, 2.31 mmol) was treated immediately with this mesylate and TEA (0.34 mL, 2.46 mmol, d 0.726). Column chromatography (silica gel, 4% MeOH in EtOAc with 1% TEA) afforded the title compound as a pale yellow oil: LC/MS t_{R} 0.45 min; MS (ES+) m/z 205; ¹H NMR δ_{H} (250 MHz, CDCl₃) 7.19-7.36 (5H, m), 3.36 (1H, q), 2.76-3.01 (4H, m), 2.64 (1H, app dq), 1.90 (1H, app td), 1.76 (1H, br s), 1.68 (1H, t), 1.37 (3H, d), 1.05 (3H, d).

### Example 30

### [5-(4-Methoxyphenyl)-6-methyl-7-(trifluoromethyl)pyrazolo[1,5-a]pyrimidin-3-yl][(2R)-2-methyl-4-[(1S)-1-phenylethyl]-1-piperazinyl]methanone (27)

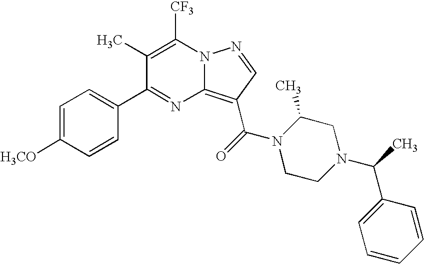

Carboxylic acid 19 (35 mg, 0.10 mmol) was treated with HATU (53 mg, 0.14 mmol), DIPEA (25.6 μL, 0.14 mmol, d 0.742) and piperazine 28 (21 mg, 0.10 mmol) in accordance with Method G to afford the title compound after column chromatography (silica gel, 30-50% EtOAc in heptanes) as a vitreous yellow solid: LC/MS t_{R} 1.68 min; MS (ES+) m/z 538, 560; ¹H NMR δ_{H} (360 MHz, CDCl₃) 8.44 (1H, s), 7.53 (2H, d), 7.21-7.40 (5H, m), 7.05 (2H, d), 4.17-4.89 (1H, br s), 3.95 (3H, s), 3.44 (1H, br s), 3.35 (1H, q), 3.04 (1H, br s), 2.56 (3H, q), 2.56 (1H, obs br s), 2.15-2.26 (2H, m), 1.36 (6H, 2 d).

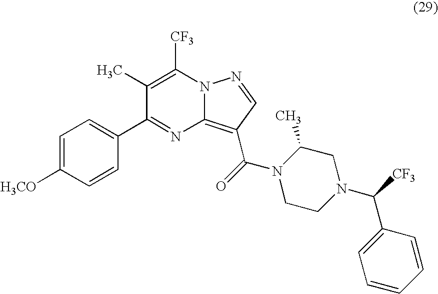

### Example 31

### (3R)-3-Methyl-1-[(1R)-2,2,2-trifluoro-1-phenylethyl]piperazine (30)

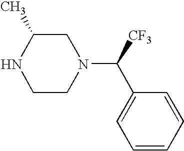

In accordance with Method Q (second alternative procedure), (S)-(+)-α-(trifluoromethyl)benzyl alcohol (200 mg, 1.14 mmol) was treated with trifluoromethanesulfonic anhydride (0.33 mL, 1.99 mmol, d 1.677) and 2,6-lutidine (0.25 mL, 2.16 mmol, d 0.726) to fashion the corresponding triflate. (R)-(−)-2-Methylpiperazine (159 mg, 1.59 mmol) was treated immediately with this triflate and TEA (0.24 mL, 1.70 mmol, d 0.726) to afford the title compound as a brown oil: LC/MS t_{R} 1.21 min; MS (ES+) m/z 259; ¹H NMR δ_{H} (360 MHz, CDCl₃) 7.37 (5H, br s), 4.01 (1H, q), 2.82-2.97 (4H, m), 2.78 (1H, app dq), 2.04-2.18 (2H, m), 1.01 (3H, d).

### Example 32

### [5-(4-Methoxyphenyl)-6-methyl-7-(trifluoromethyl)pyrazolo[1,5-a]pyrimidin-3-yl][(2R)-2-methyl-4-[(1R)-2,2,2-trifluoro-1-phenylethyl]-1-piperazinyl]methanone (29)

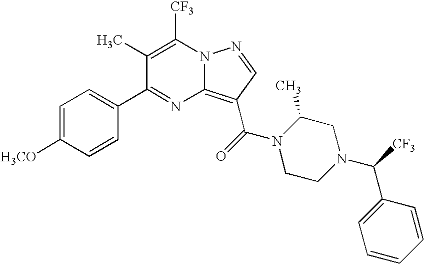

Carboxylic acid 19 (40 mg, 0.11 mmol) was treated with HATU (61 mg, 0.16 mmol), DIPEA (28 μL, 0.16 mmol, d 0.742) and piperazine 30 (41 mg, 0.16 mmol) in accordance with Method G to afford the title compound after column chromatography (silica gel, 30% EtOAc in heptanes) as a vitreous yellow solid: LC/MS t_{R} 2.64 min; MS (ES+) m/z 592; ¹H NMR δ_{H} (360 MHz, CDCl₃) 8.42 (1H, s), 7.48 (2H, d), 7.29-7.41 (5H, m), 7.03 (2H, d), 4.04 (1H, q), 3.96-4.89 (2H, br s), 3.93 (3H, s), 3.44 (1H, br s), 2.94 (1H, br s), 2.71 (1H, br d), 2.64 (1H, t), 2.55 (3H, q), 2.42 (1H, dd), 1.38 (3H, d).

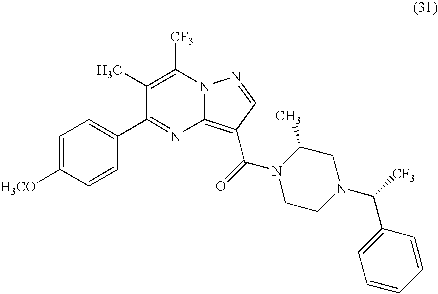

### Example 33

### (3R)-3-Methyl-1-[(1S)-2,2,2-trifluoro-1-phenylethyl]piperazine (32)

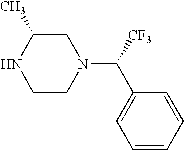

In accordance with Method Q (second alternative procedure), (R)-(−)-α-(trifluoromethyl)benzyl alcohol (200 mg, 1.14 mmol) was treated with trifluoromethanesulfonic anhydride (0.33 mL, 1.99 mmol, d 1.677) and 2,6-lutidine (0.25 mL, 2.16 mmol, d 0.726) to fashion the corresponding triflate. (R)-(−)-2-Methylpiperazine (159 mg, 1.59 mmol) was treated immediately with this triflate and TEA (0.24 mL, 1.70 mmol, d 0.726) to afford the title compound as a brown oil: LC/MS t_{R} 1.28 min; MS (ES+) m/z 259; ¹H NMR δ_{H} (360 MHz, CDCl₃) 7.37 (5H, br s), 4.02 (1H, q), 2.94 (1H, d), 2.92 (1H, t), 2.73-2.89 (3H, m), 2.43 (1H, ddd), 1.81 (1H, app td), 1.19 (3H, d).

### Example 34

### [5-(4-Methoxyphenyl)-6-methyl-7-(trifluoromethyl)pyrazolo[1,5-a]pyrimidin-3-yl][(2R)-2-methyl-4-[(1S)-2,2,2-trifluoro-1-phenylethyl]-1-piperazinyl]methanone (31)

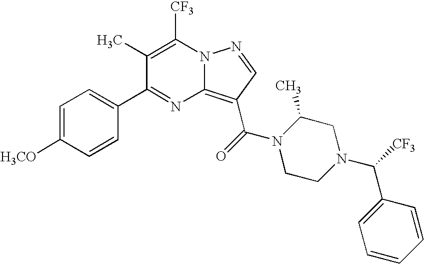

Carboxylic acid 19 (40 mg, 0.11 mmol) was treated with HATU (61 mg, 0.16 mmol), DIPEA (28 μL, 0.16 mmol, d 0.742) and piperazine 32 (41 mg, 0.16 mmol) in accordance with Method G to afford the title compound after column chromatography (silica gel, 30% EtOAc in heptanes) as a vitreous yellow solid: LC/MS t_{R} 2.65 min; MS (ES+) m/z 592; ¹H NMR δ_{H} (360 MHz, CDCl₃) 8.41 (1H, s), 7.48 (2H, d), 7.29-7.41 (5H, m), 7.03 (2H, d), 4.05 (1H, q), 3.97-5.00 (2H, br s), 3.93 (3H, s), 3.33 (1H, br s), 2.62-2.93 (3H, br m), 2.54 (3H, q), 2.33 (1H, app td), 1.35 (3H, d).

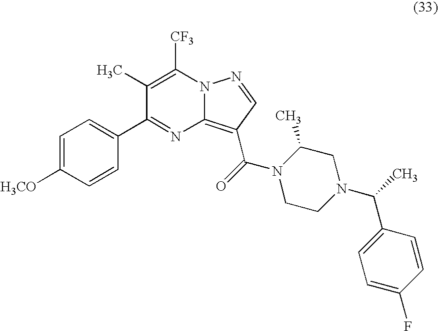

### Example 35

### (3R)-1-[(1R)-1-(4-Fluorophenyl)ethyl]-3-methylpiperazine (34)

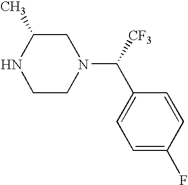

In accordance with Method Q (first alternative procedure), (S)-(−)-4-fluoro-α-methylbenzyl alcohol (165 mg, 1.18 mmol) was treated with MsCl (0.12 mL, 1.54 mmol, d 1.48) and TEA (0.34 mL, 2.46 mmol, d 0.726) to fashion the corresponding mesylate. A portion of the isolated mesylate (50 mg, 0.23 mmol) was treated with (R)-(−)-2-methylpiperazine (27 mg, 0.28 mmol) and TEA (48 μL, 0.35 mmol, d 0.726). Column chromatography (silica gel, 3-4% MeOH in EtOAc with 1% TEA) afforded the title compound as a pale yellow oil: LC/MS t_{R} 0.67 min; MS (ES+) m/z 223; ¹H NMR δ_{H} (360 MHz, CDCl₃) 7.26 (2H, dd), 7.01 (2H, t), 3.40 (1H, q), 3.28 (1H, d), 2.93-3.08 (3H, m), 2.67 (1H, d), 2.31 (1H, app td), 1.93 (1H, t), 1.33 (3H, d), 1.18 (3H, d).

### Example 36

### [(2R)-4-[(1R)-1-(4-Fluorophenyl)ethyl]-2-methyl-1-piperazinyl][5-(4-methoxyphenyl)-6-methyl-7-(trifluoromethyl)pyrazolo[1,5-a]pyrimidin-3-yl]methanone (33)

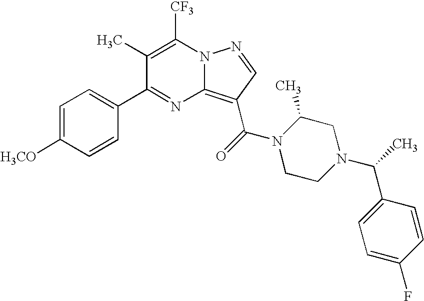

Carboxylic acid 19 (44 mg, 0.13 mmol) was treated with HATU (53 mg, 0.14 mmol), DIPEA (24.8 μL, 0.14 mmol, d 0.742) and piperazine 34 (28 mg, 0.13 mmol) in accordance with Method G to afford the title compound after column chromatography (silica gel, 50% EtOAc in heptanes) as a vitreous yellow solid: LC/MS t_{R} 1.73 min; MS (ES+) m/z 556; ¹H NMR δ_{H} (360 MHz, CDCl₃) 8.42 (1H, s), 7.50 (2H, d), 7.28 (2H, dd), 7.03 (2H, d), 6.99 (2H, t), 3.97-4.87 (1H, br s), 3.93 (3H, s), 3.53-3.96 (1H, br s), 3.38 (1H, q), 3.29 (1H, br s), 2.54 (3H, q), 2.37-2.85 (2H, br m), 2.31 (1H, dd), 2.12 (1H, app td), 1.39 (3H, d), 1.29 (3H, d).

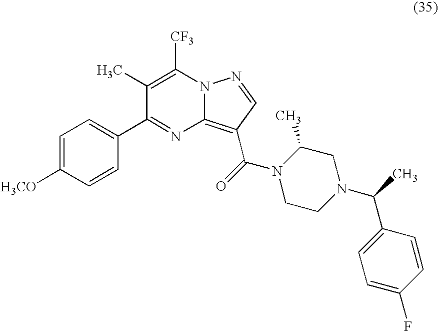

### Example 37

### (3R)-1-[(is)-1-(4-Fluorophenyl)ethyl]-3-methylpiperazine (36)

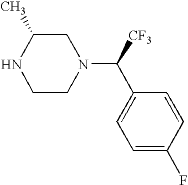

In accordance with Method Q (first alternative procedure), (R)-(+)-4-fluoro-α-methylbenzyl alcohol (165 mg, 1.18 mmol) was treated with MsCl (0.12 mL, 1.54 mmol, d 1.48) and TEA (0.34 mL, 2.46 mmol, d 0.726) to fashion the corresponding mesylate. (R)-(−)-2-Methylpiperazine (232 mg, 2.31 mmol) was treated immediately with this mesylate and TEA (0.34 mL, 2.46 mmol, d 0.726). Column chromatography (silica gel, 4% MeOH in EtOAc with 1% TEA) afforded the title compound as a pale yellow oil: LC/MS t_{R} 0.67 min; MS (ES+) m/z 223; ¹H NMR δ_{H} (250 MHz, CDCl₃) 7.25 (2H, dd), 6.98 (2H, t), 3.34 (1H, q), 2.71-2.99 (4H, m), 2.59 (1H, app dq), 1.92 (1H, s), 1.89 (1H, app td), 1.67 (1H, t), 1.32 (3H, d), 1.04 (3H, d).

### Example 38

### [(2R)-4-[(1S)-1-(4-Fluorophenyl)ethyl]-2-methyl-1-piperazinyl][5-(4-methoxyphenyl)-6-methyl-7-(trifluoromethyl)pyrazolo[1,5-a]pyrimidin-3-yl]methanone (35)

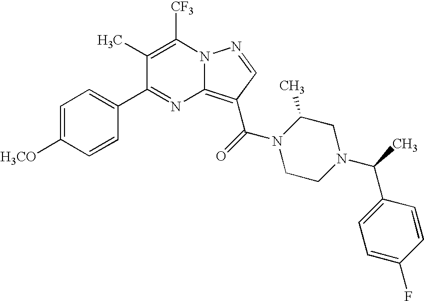

Carboxylic acid 19 (35 mg, 0.10 mmol) was treated with HATU (53 mg, 0.14 mmol), DIPEA (25.6 μL, 0.14 mmol, d 0.742) and piperazine 36 (22 mg, 0.10 mmol) in accordance with Method G to afford the title compound after column chromatography (silica gel, 50% EtOAc in heptanes) as a vitreous yellow solid: LC/MS t_{R} 1.75 min; MS (ES+) m/z 556; ¹H NMR δ_{H} (360 MHz, CDCl₃) 8.42 (1H, s), 7.51 (2H, d), 7.26 (2H, dd), 7.03 (2H, d), 6.99 (2H, t), 3.93 (3H, s), 3.71-4.93 (2H, br s), 3.36 (1H, br s), 3.31 (1H, q), 2.99 (1H, br s), 2.54 (3H, q), 2.50 (1H, br s), 2.10-2.23 (2H, m), 1.31 (2×3H, d).

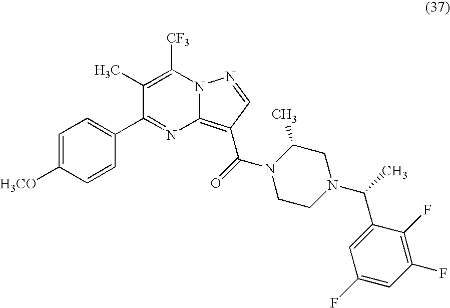

### Example 39

### (αS)-2,3,5-Trifluoro-α-methylbenzenemethanol (38)

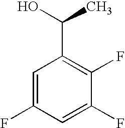

2,3,5-Trifluoroacetophenone (0.20 g, 1.15 mmol) was treated with (−)-DIP-Cl (0.44 g, 1.38 mmol) in accordance with Method P to give the title compound admixed with α-pinene after column chromatography (silica gel, 10% EtOAc in heptanes) as a pale yellow oil: LC/MS t_{R} 1.69 min; ¹H NMR δ_{H} (250 MHz, CDCl₃) 7.05 (1H, m), 6.83 (1H, m), 5.22 (1H, br s), 1.51 (3H, d).

### Example 40

### (3R)-3-Methyl-1-[(1R)-1-(2,3,5-trifluorophenyl)ethyl]piperazine (39)

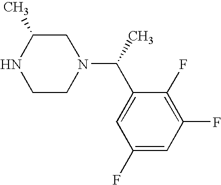

In accordance with Method Q, phenethyl alcohol 38 (200 mg, 0.57 mmol, ca. 50% purity) was treated with MsCl (57 μL, 0.74 mmol, d 1.48) and TEA (0.16 mL, 1.14 mmol, d 0.726) to fashion the corresponding mesylate. (R)-(−)-2-Methylpiperazine (57 mg, 0.57 mmol) was treated immediately with this mesylate and 2,2,6,6-tetramethylpiperidine (98 μL, 0.58 mmol, d 0.837) to afford the title compound as a colorless oil: LC/MS t_{R} 1.25 min; MS (ES+) m/z 259; ¹H NMR δ_{H} (360 MHz, CDCl₃) 6.93 (1H, m), 6.79 (1H, m), 3.81 (1H, q), 2.87-3.06 (3H, m), 2.78 (1H, ddd), 2.61 (1H, app dt), 2.05 (1H, app td), 1.56 (1H, t), 1.32 (3H, d), 0.96 (3H, d).

### Example 41

### [5-(4-Methoxyphenyl)-6-methyl-7-(trifluoromethyl)pyrazolo[1,5-a]pyrimidin-3-yl][(2R)-2-methyl-4-[(1R)-1-(2,3,5-trifluorophenyl)ethyl]-1-piperazinyl]methanone (37)

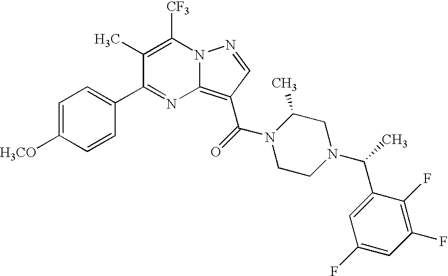

In accordance with Method H, carboxylic acid 19 (45 mg, 0.13 mmol) was converted to the analogous acid chloride via treatment with oxalyl chloride (16 μL, 0.18 mmol, d 1.455) and catalytic DMF (5 μL) then treated with DIPEA (40 μL, 0.23 mmol, d 0.742) and piperazine 39 (42 mg, 0.16 mmol). Column chromatography (silica gel, 20-30% EtOAc in heptanes) afforded the title compound as a vitreous yellow solid: LC/MS t_{R} 2.35 min; MS (ES+) m/z 592; ¹H NMR δ_{H} (360 MHz, CDCl₃) 8.43 (1H, s), 7.50 (2H, d), 7.04 (2H, d), 6.94 (1H, m), 6.81 (1H, m), 3.92 (3H, s), 3.81 (1H, q), 3.72-4.99 (2H, br s), 3.27 (1H, br s), 2.87 (1H, br s), 2.66 (1H, br s), 2.55 (3H, q), 2.37 (1H, dd), 2.09 (1H, t), 1.41 (3H, d), 1.32 (3H, d).

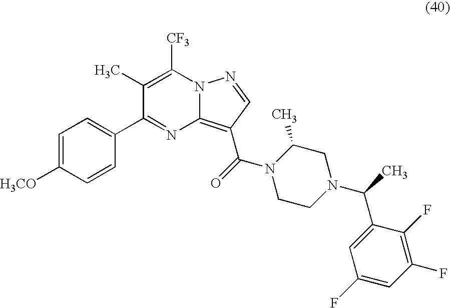

### Example 42

### (αR)-2,3,5-Trifluoro-α-methylbenzenemethanol (41)

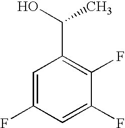

2,3,5-Trifluoroacetophenone (0.40 g, 2.30 mmol) was treated with (+)-DIP-Cl (0.89 g, 2.76 mmol) in accordance with Method P to give the title compound admixed with α-pinene after column chromatography (silica gel, 5-10% EtOAc in heptanes) as a pale yellow oil: LC/MS t_{R} 1.68 min; ¹H NMR δ_{H} (250 MHz, CDCl₃) 7.04 (1H, m), 6.83 (1H, m), 5.21 (1H, br s), 2.14 (1H, br s), 1.51 (3H, d).

### Example 43

### (3R)-3-Methyl-1-[(1S)-1-(2,3,5-trifluorophenyl)ethyl]piperazine (42)

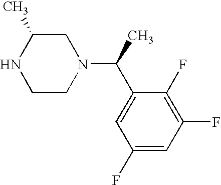

In accordance with Method Q, phenethyl alcohol 41 (362 mg, 1.44 mmol, ca. 70% purity) was treated with MsCl (145 μL, 1.87 mmol, d 1.48) and TEA (0.40 mL, 2.89 mmol, d 0.726) to fashion the corresponding mesylate. (R)-(−)-2-Methylpiperazine (144 mg, 1.44 mmol) was treated immediately with this mesylate and 2,2,6,6-tetramethylpiperidine (0.25 mL, 1.48 mmol, d 0.837) to afford the title compound as a colorless oil: LC/MS t_{R} 1.20 min; MS (ES+) m/z 259; ¹H NMR δ_{H} (360 MHz, CDCl₃) 6.92 (1H, m), 6.78 (1H, m), 3.82 (1H, q), 2.73-3.00 (4H, m), 2.62 (1H, app dt), 1.89 (1H, app td), 1.69 (1H, t), 1.33 (3H, d), 1.04 (3H, d).

### Example 44

### [5-(4-Methoxyphenyl)-6-methyl-7-(trifluoromethyl)pyrazolo[1,5-a]pyrimidin-3-yl][(2R)-2-methyl-4-[(1S)-1-(2,3,5-trifluorophenyl)ethyl]-1-piperazinyl]methanone (40)

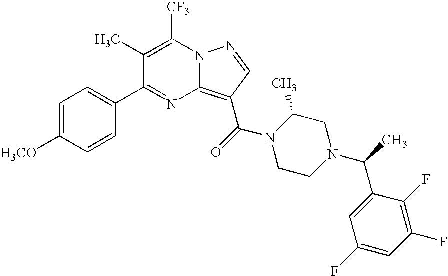

Carboxylic acid 19 (30 mg, 0.085 mmol) was treated with HATU (39 mg, 0.10 mmol), DIPEA (18 μL, 0.10 mmol, d 0.742) and piperazine 42 (26 mg, 0.10 mmol) in accordance with Method G to afford the title compound after column chromatography (silica gel, 20-30% EtOAc in heptanes) as a vitreous yellow solid: LC/MS t_{R} 2.31 min; MS (ES+) m/z 592; ¹H NMR δ_{H} (360 MHz, CDCl₃) 8.43 (1H, s), 7.50 (2H, d), 7.04 (2H, d), 6.94 (1H, m), 6.81 (1H, m), 3.92 (3H, s), 3.86-5.07 (2H, br s), 3.81 (1H, q), 3.37 (1H, br s), 3.03 (1H, br s), 2.55 (3H, q), 2.51 (1H, obs br s), 2.16-2.27 (2H, m), 1.33 (2×3H, d).

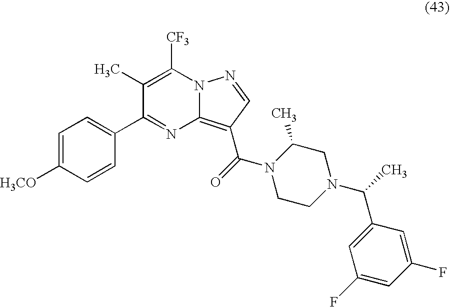

### Example 45

### (αS)-3,5-Difluoro-α-methylbenzenemethanol (44)

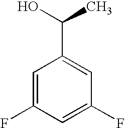

3,5-Difluoroacetophenone (0.40 g, 2.56 mmol) was treated with (−)-DIP-Cl (1.07 g, 3.33 mmol) in accordance with Method P to give the title compound admixed with α-pinene after column chromatography (silica gel, 10-30% EtOAc in heptanes) as a pale yellow oil: ¹H NMR δ_{H} (250 MHz, CDCl₃) 6.81 (2H, m), 6.61 (1H, app tt), 4.79 (1H, q), 1.39 (3H, d).

### Example 46

### (3R)-1-[(1R)-1-(3,5-Difluorophenyl)ethyl]-3-methylpiperazine (45)

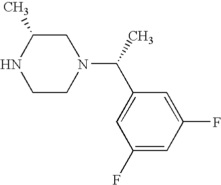

In accordance with Method Q (first alternative procedure), phenethyl alcohol 44 (250 mg, 0.73 mmol, ca. 70% purity) was treated with MsCl (58 μL, 0.75 mmol, d 1.48) and TEA (0.20 mL, 1.46 mmol, d 0.726) to fashion the corresponding mesylate. (R)-(−)-2-Methylpiperazine (73 mg, 0.73 mmol) was treated immediately with this mesylate and TEA (0.20 mL, 1.46 mmol, d 0.726) to afford the title compound as a pale yellow oil: LC/MS t_{R} 1.13 min; MS (ES+) m/z 241.

### Example 47

### [(2R)-4-[(1R)-1-(3,5-Difluorophenyl)ethyl]-2-methyl-1-piperazinyl][5-(4-methoxyphenyl)-6-methyl-7-(trifluoromethyl)pyrazolo[1,5-a]pyrimidin-3-yl]methanone (43)

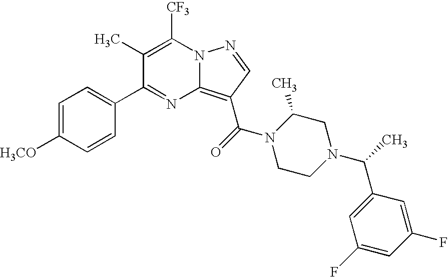

Carboxylic acid 19 (72 mg, 0.20 mmol) was treated with HATU (93 mg, 0.25 mmol), DIPEA (43 μL, 0.25 mmol, d 0.742) and piperazine 45 (59 mg, 0.25 mmol) in accordance with Method G. Column chromatography (silica gel, 40% EtOAc in heptanes, then repeated using 30% EtOAc in heptanes) afforded the title compound as a vitreous yellow solid: LC/MS t_{R} 2.02 min; MS (ES+) m/z 574; ¹H NMR δ_{H} (360 MHz, CDCl₃) 8.43 (1H, s), 7.50 (2H, d), 7.03 (2H, d), 6.89 (2H, m), 6.68 (1H, tt), 3.92 (3H, s), 3.62-5.01 (2H, br s), 3.36 (1H, q), 3.30 (1H, br s), 2.55 (3H, q), 2.42-2.83 (2H, br m), 2.34 (1H, dd), 2.18 (1H, app td), 1.40 (3H, d), 1.28 (3H, d).

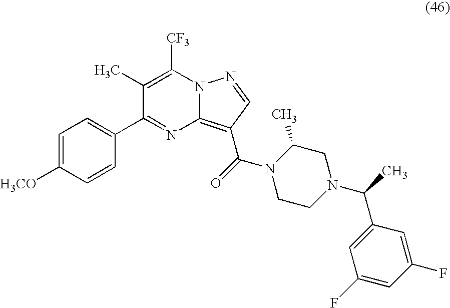

### Example 48

### (αR)-3,5-Difluoro-α-methylbenzenemethanol (47)

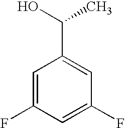

3,5-Difluoroacetophenone (0.40 g, 2.56 mmol) was treated with (+)-DIP-Cl (0.99 g, 3.08 mmol) in accordance with Method P to give the title compound admixed with α-pinene after column chromatography (silica gel, 10-20% EtOAc in heptanes) as a pale yellow oil: LC/MS t_{R} 1.60 min; ¹H NMR δ_{H} (250 MHz, CDCl₃) 6.82 (2H, m), 6.61 (1H, tt), 4.79 (1H, q), 1.96 (1H, br s), 1.39 (3H, d).

### Example 49

### (3R)-1-[(1S)-1-(3,5-Difluorophenyl)ethyl]-3-methylpiperazine (48)

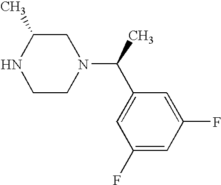

In accordance with Method Q (first alternative procedure), phenethyl alcohol 47 (614 mg, 1.56 mmol, ca. 40% purity) was treated with MsCl (157 μL, 2.02 mmol, d 1.48) and TEA (0.43 mL, 3.11 mmol, d 0.726) to fashion the corresponding mesylate. (R)-(−)-2-Methylpiperazine (156 mg, 1.56 mmol) was treated immediately with this mesylate and TEA (0.22 mL, 1.60 mmol, d 0.726) to afford the title compound as a pale yellow oil: LC/MS t_{R} 1.13 min; MS (ES+) m/z 241; ¹H NMR δ_{H} (360 MHz, CDCl₃) 6.84 (2H, m), 6.63 (1H, m), 3.31 (1H, q), 2.74-2.90 (4H, m), 2.57 (1H, app dq), 1.92 (1H, app td), 1.65 (1H, t), 1.28 (3H, d), 1.01 (3H, d).

### Example 50

### [(2R)-4-[(1S)-1-(3,5-Difluorophenyl)ethyl]-2-methyl-1-piperazinyl][5-(4-methoxyphenyl)-6-methyl-7-(trifluoromethyl)pyrazolo[1,5-a]pyrimidin-3-yl]methanone (46)

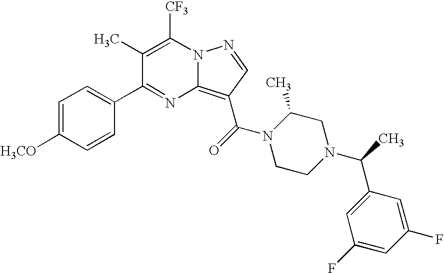

Carboxylic acid 19 (60 mg, 0.17 mmol) was treated with HATU (78 mg, 0.20 mmol), DIPEA (35 μL, 0.20 mmol, d 0.742) and piperazine 48 (49 mg, 0.20 mmol) in accordance with Method G. Reverse phase preparative HPLC (5-95% MeCN in water) afforded the title compound as a vitreous yellow solid: LC/MS t_{R} 2.00 min; MS (ES+) m/z 574; ¹H NMR δ_{H} (360 MHz, CDCl₃) 8.43 (1H, s), 7.51 (2H, d), 7.04 (2H, d), 6.86 (2H, m), 6.68 (1H, tt), 3.92 (3H, s), 3.88-4.96 (2H, br s), 3.39 (1H, br s), 3.30 (1H, q), 2.96 (1H, br s), 2.55 (3H, q), 2.51 (1H, obs br s), 2.25 (1H, dd), 2.18 (1H, app td), 1.34 (3H, d), 1.29 (3H, d).

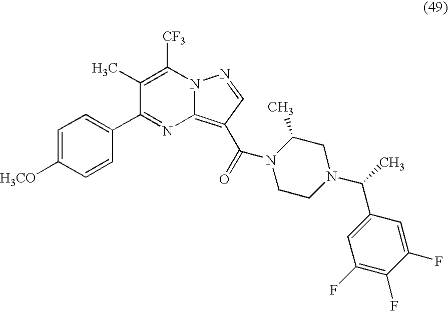

### Example 51

### (αS)-3,4,5-Trifluoro-α-methylbenzenemethanol (50)

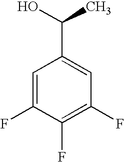

3,4,5-Trifluoroacetophenone (0.40 g, 2.30 mmol) was treated with (−)-DIP-Cl (0.96 g, 2.99 mmol) in accordance with Method P to give the title compound admixed with α-pinene after column chromatography (silica gel, 10-30% EtOAc in heptanes) as a pale yellow oil: ¹H NMR δ_{H} (250 MHz, CDCl₃) 6.99 (2H, m), 4.86 (1H, m), 1.46 (3H, d).

### Example 52

### (3R)-3-Methyl-1-[(1R)-1-(3,4,5-trifluorophenyl)ethyl]piperazine (51)

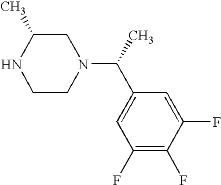

In accordance with Method Q (first alternative procedure), phenethyl alcohol 50 (0.46 g, 1.57 mmol, ca. 60% purity) was treated with MsCl (122 μL, 1.58 mmol, d 1.48) and TEA (0.31 mL, 2.21 mmol, d 0.726) to fashion the corresponding mesylate. (R)-(−)-2-Methylpiperazine (190 mg, 1.89 mmol) was treated immediately with this mesylate and TEA (0.31 mL, 2.21 mmol, d 0.726) to afford the title compound as a pale yellow oil: LC/MS t_{R} 1.23 min; MS (ES+) m/z 259.

### Example 53

### [5-(4-Methoxyphenyl)-6-methyl-7-(trifluoromethyl)pyrazolo[1,5-a]pyrimidin-3-yl][(2R)-2-methyl-4-[(1R)-1-(3,4,5-trifluorophenyl)ethyl]-1-piperazinyl]methanone (49)

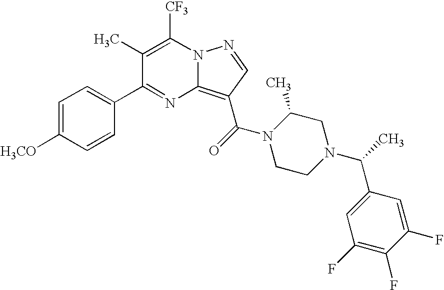

Carboxylic acid 19 (31 mg, 0.089 mmol) was treated with HATU (37 mg, 0.10 mmol), DIPEA (19 μL, 0.11 mmol, d 0.742) and piperazine 51 (23 mg, 0.089 mmol) in accordance with Method G. Reverse phase preparative HPLC (5-95% MeCN in water) afforded the title compound as a vitreous yellow solid: LC/MS t_{R} 2.21 min; MS (ES+) m/z 592; ¹H NMR δ_{H} (360 MHz, CDCl₃) 8.43 (1H, s), 7.51 (2H, d), 7.03 (2H, d), 6.98 (2H, m), 3.92 (3H, s), 3.61-5.15 (2H, br s), 3.33 (1H, q), 3.29 (1H, br s), 2.54 (3H, q), 2.41-2.80 (2H, br m), 2.33 (1H, dd), 2.19 (1H, app td), 1.39 (3H, d), 1.25 (3H, d).

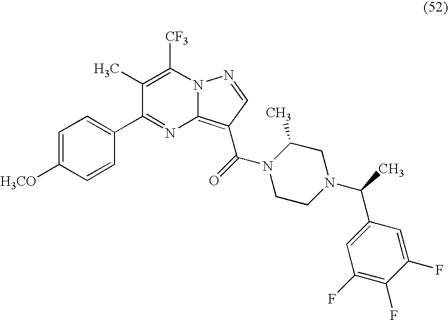

### Example 54

### (αR)-3,4,5-Trifluoro-α-methylbenzenemethanol (53)

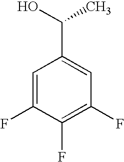

3,4,5-Trifluoroacetophenone (0.40 g, 2.30 mmol) was treated with (+)-DIP-Cl (0.89 g, 2.76 mmol) in accordance with Method P to give the title compound admixed with α-pinene after column chromatography (silica gel, 10-20% EtOAc in heptanes) as a pale yellow oil: LC/MS t_{R} 1.70 min; ¹H NMR δ_{H} (360 MHz, CDCl₃) 7.01 (2H, m), 4.85 (1H, m), 1.47 (3H, d).

### Example 55

### (3R)-3-Methyl-1-[(1S)-1-(3,4,5-trifluorophenyl)ethyl]piperazine (54)

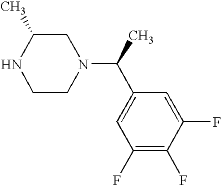

In accordance with Method Q, phenethyl alcohol 53 (307 mg, 1.26 mmol, ca. 72% purity) was treated with MsCl (126 μL, 1.63 mmol, d 1.48) and TEA (0.35 mL, 2.51 mmol, d 0.726) to fashion the corresponding mesylate. (R)-(−)-2-Methylpiperazine (126 mg, 1.26 mmol) was treated immediately with this mesylate and 2,2,6,6-tetramethylpiperidine (0.22 mL, 1.29 mmol, d 0.837) to afford the title compound as a pale yellow oil: LC/MS t_{R} 1.19 min; MS (ES+) m/z 259; ¹H NMR δ_{H} (360 MHz, CDCl₃) 6.93 (2H, m), 3.26 (1H, q), 2.74-2.90 (4H, m), 2.53 (1H, app dq), 1.93 (1H, app td), 1.64 (1H, t), 1.24 (3H, d), 1.00 (3H, d).

### Example 56

### [5-(4-Methoxyphenyl)-6-methyl-7-(trifluoromethyl)pyrazolo[1,5-a]pyrimidin-3-yl][(2R)-2-methyl-4-[(1S)-1-(3,4,5-trifluorophenyl)ethyl]-1-piperazinyl]methanone (52)

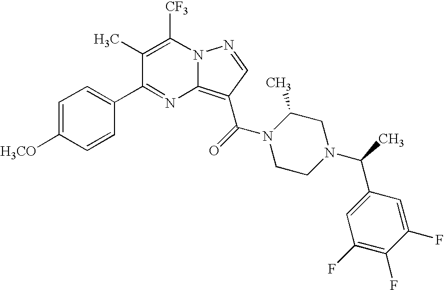

Carboxylic acid 19 (31 mg, 0.089 mmol) was treated with HATU (37 mg, 0.10 mmol), DIPEA (19 μL, 0.11 mmol, d 0.742) and piperazine 54 (23 mg, 0.089 mmol) in accordance with Method G. Reverse phase preparative HPLC (5-95% MeCN in water) afforded the title compound as a vitreous yellow solid: LC/MS t_{R} 2.19 min; MS (ES+) m/z 592; ¹H NMR δ_{H} (360 MHz, CDCl₃) 8.43 (1H, s), 7.51 (2H, d), 7.03 (2H, d), 6.96 (2H, m), 3.92 (3H, s), 3.75-5.10 (2H, br s), 3.34 (1H, br s), 3.26 (1H, q), 2.93 (1H, br s), 2.54 (3H, q), 2.46 (1H, br s), 2.25 (1H, dd), 2.18 (1H, app td), 1.33 (3H, d), 1.27 (3H, d).

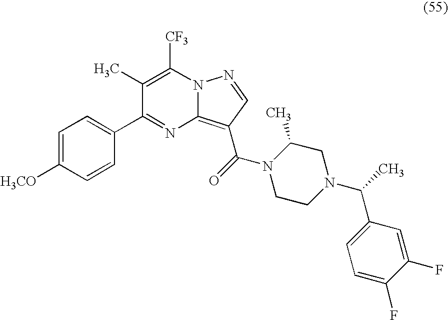

### Example 57

### (αS)-3,4-Difluoro-α-methylbenzenemethanol (56)

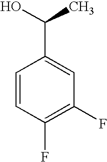

3,4-Difluoroacetophenone (0.40 g, 2.56 mmol) was treated with (−)-DIP-Cl (0.99 g, 3.07 mmol) in accordance with Method P to give the title compound admixed with α-pinene after column chromatography (silica gel, 10-30% EtOAc in heptanes) as a colorless oil: LC/MS t_{R} 1.55 min; ¹H NMR δ_{H} (250 MHz, CDCl₃) 7.00-7.30 (3H, m), 4.88 (1H, app qd), 1.48 (3H, d).

### Example 58

### (3R)-1-[(1R)-1-(3,4-Difluorophenyl)ethyl]-3-methylpiperazine (57)

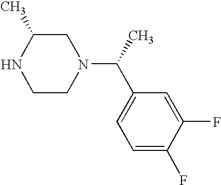

In accordance with Method Q, phenethyl alcohol 56 (0.83 g, 2.64 mmol, ca. 50% purity) was treated with MsCl (0.27 mL, 3.43 mmol, d 1.48) and TEA (0.74 mL, 5.28 mmol, d 0.726) to fashion the corresponding mesylate. (R)-(−)-2-Methylpiperazine (264 mg, 2.64 mmol) was treated immediately with this mesylate and 2,2,6,6-tetramethylpiperidine (0.46 mL, 2.71 mmol, d 0.837) to afford the title compound after column chromatography (silica gel, 0-5% MeOH in EtOAc) as a pale yellow oil: LC/MS t_{R} 1.02 min; MS (ES+) m/z 241.

### Example 59

### [(2R)-4-[(1R)-1-(3,4-Difluorophenyl)ethyl]-2-methyl-1-piperazinyl][5-(4-methoxyphenyl)-6-methyl-7-(trifluoromethyl)pyrazolo[1,5-a]pyrimidin-3-yl]methanone (55)

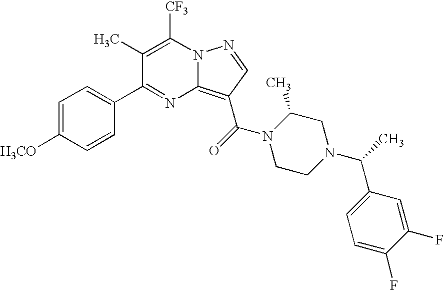

Carboxylic acid 19 (44 mg, 0.125 mmol) was treated with HATU (52 mg, 0.14 mmol), DIPEA (25 μL, 0.15 mmol, d 0.742) and piperazine 57 (30 mg, 0.125 mmol) in accordance with Method G. Column chromatography (silica gel, 10-50% EtOAc in heptanes) followed by reverse phase preparative HPLC (0.1% TFA, 5-95% MeCN in water) afforded the TFA salt of the title compound as a vitreous yellow solid: LC/MS t_{R} 1.84 min; MS (ES+) m/z 574; ¹H NMR δ_{H} (360 MHz, CDCl₃) 8.51 (1H, s), 7.45 (2H, d), 7.14 (2H, d), 6.87-7.19 (3H, obs m), 5.02 (1H, br s), 3.95 (3H, s), 3.69-4.51 (3H, br m), 3.43 (1H, br d), 3.43 (1H, obs br s), 2.85 (2H, m), 2.53 (3H, q), 1.70 (3H, d), 1.50 (3H, d).

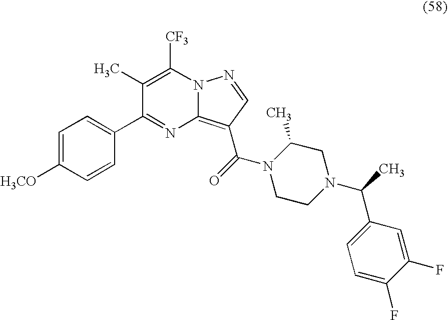

### Example 60

### (αR)-3,4-Difluoro-α-methylbenzenemethanol (59)

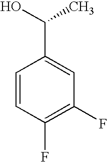

3,4-Difluoroacetophenone (0.40 g, 2.56 mmol) was treated with (+)-DIP-Cl (1.15 g, 3.59 mmol) in accordance with Method P to give the title compound after column chromatography (silica gel, 0-30% EtOAc in heptanes) as a colorless oil: LC/MS t_{R} 1.56 min; ¹H NMR δ_{H} (250 MHz, CDCl₃) 7.00-7.27 (3H, m), 4.87 (1H, app qd), 1.93 (1H, br d), 1.47 (3H, d).

### Example 61

### (3R)-1-[(1S)-1-(3,4-Difluorophenyl)ethyl]-3-methylpiperazine (60)

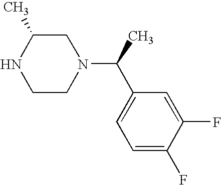

In accordance with Method Q (first alternative procedure), phenethyl alcohol 59 (228 mg, 1.44 mmol) was treated with MsCl (145 μL, 1.88 mmol, d 1.48) and TEA (0.40 mL, 2.89 mmol, d 0.726) to fashion the corresponding mesylate. (R)-(−)-2-Methylpiperazine (144 mg, 1.44 mmol) was treated immediately with this mesylate and TEA (0.21 mL, 1.49 mmol, d 0.726) to afford the title compound as an orange oil: LC/MS t_{R} 0.92 min; MS (ES+) m/z 241; ¹H NMR δ_{H} (250 MHz, CDCl₃) 6.87-7.19 (3H, m), 3.29 (1H, q), 2.68-2.94 (4H, m), 2.55 (1H, m), 2.07 (1H, br s), 1.91 (1H, app td), 1.65 (1H, t), 1.27 (3H, d), 1.01 (3H, d).

### Example 62

### [(2R)-4-[(is)-1-(3,4-Difluorophenyl)ethyl]-2-methyl-1-piperazinyl][5-(4-methoxyphenyl)-6-methyl-7-(trifluoromethyl)pyrazolo[1,5-a]pyrimidin-3-yl]methanone (58)

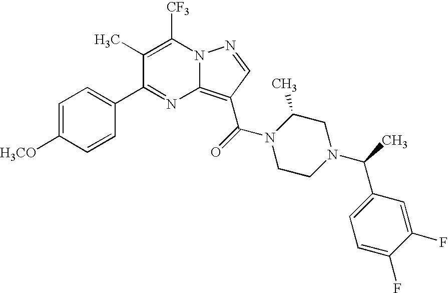

Carboxylic acid 19 (60 mg, 0.17 mmol) was treated with HATU (78 mg, 0.20 mmol), DIPEA (36 μL, 0.21 mmol, d 0.742) and piperazine 60 (58 mg, 0.24 mmol) in accordance with Method G to afford the title compound after column chromatography (silica gel, 40% EtOAc in heptanes) as a vitreous yellow solid: LC/MS t_{R} 1.84 min; MS (ES+) m/z 574; ¹H NMR δ_{H} (250 MHz, CDCl₃) 8.42 (1H, s), 7.50 (2H, d), 7.03 (2H, d), 6.94-7.22 (3H, obs m), 3.92 (3H, s), 3.74-5.31 (2H, br s), 3.37 (1H, br s), 3.29 (1H, q), 2.97 (1H, br s), 2.54 (3H, q), 2.49 (1H, obs br s), 2.08-2.27 (2H, m), 1.32 (3H, d), 1.29 (3H, d).

### Examples of Compounds Prepared Employing Routes General Routes A, B, and C

### Alternate route to [5-(4-Methoxyphenyl)-6-methyl-7-(trifluoromethyl)pyrazolo[1,5-a]pyrimidin-3-yl][(2R)-2-methyl-4-[(1R)-1-(2,3,5-trifluorophenyl)ethyl]-1-piperazinyl]methanone (37) and [5-(4-Methoxyphenyl)-6-methyl-7-(trifluoromethyl)pyrazolo[1,5-a]pyrimidin-3-yl][(2R)-2-methyl-4-[(1S)-1-(2,3,5-trifluorophenyl)ethyl]-1-piperazinyl]methanone (40)

### Example 63

### 2,3,5-Trifluoro-α-methylbenzenemethanol (61)

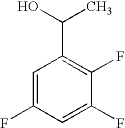

2,3,5-Trifluoroacetophenone (0.20 g, 1.15 mmol) was treated with sodium borohydride (66 mg, 1.72 mmol) in accordance with Method L to afford the title compound as a pale yellow oil: ¹H NMR δ_{H} (360 MHz, CDCl₃) 7.01 (1H, m), 6.82 (1H, m), 5.19 (1H, q), 2.35 (1H, br s), 1.49 (3H, d).

### Example 64

### (3R)-3-Methyl-1-[(1R,1S)-1-(2,3,5-trifluorophenyl)ethyl]piperazines (39) and (42)

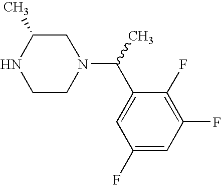

In accordance with Method M, phenethyl alcohol 61 (186 mg, 1.06 mmol) was treated with MsCl (0.11 mL, 1.37 mmol, d 1.48) and TEA (0.29 mL, 2.11 mmol, d 0.726) to fashion the corresponding mesylate. (R)-(−)-2-Methylpiperazine (81 mg, 0.81 mmol) was treated immediately with this mesylate and 2,2,6,6-tetramethylpiperidine (0.14 mL, 0.83 mmol, d 0.837) to afford the title compound as a pale yellow oil: LC/MS t_{R} 1.22 min; MS (ES+) m/z 259.

### Example 65

### (2R)-2-Methyl-4-[(1R)-1-(2,3,5-trimethylphenyl)ethyl]-1-piperazinecarboxylic Acid, 1,1-Dimethylethyl Ester (64) and (2R)-2-Methyl-4-[(1S)-1-(2,3,5-trimethylphenyl)ethyl]-1-piperazinecarboxylic Acid, 1,1-Dimethylethyl Ester (65)

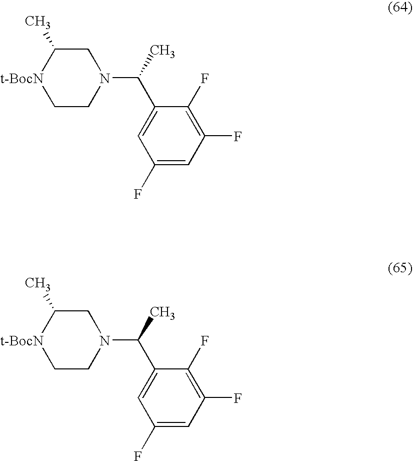

The diastereomeric mixture of piperazines 39 and 42 (166 mg, 0.64 mmol) was treated with di-tert-butyl dicarbonate (155 mg, 0.71 mmol) and DMAP (3 mg, 6.43 μmol) in accordance with Method N. Column chromatography (silica gel, 5% EtOAc in heptanes, then 2-5% EtOAc in heptanes provided the title diastereomers as colourless oils: for (64)>92% de; LC/MS t_{R} 1.98 min; MS (ES+) m/z 359; ¹H NMR δ_{H} (360 MHz, CDCl₃) 6.96 (1H, m), 6.80 (1H, m), 4.25 (1H, br s), 3.78 (1H, q), 3.73 (1H, d), 3.01 (1H, app td), 2.83 (1H, d), 2.62 (1H, d), 2.21 (1H, dd), 1.88 (1H, app td), 1.45 (9H, s), 1.31 (3H, d), 1.27 (3H, d). For (65), >90% de; LC/MS t_{R} 1.96 min; MS (ES+) m/z 359; ¹H NMR δ_{H} (360 MHz, CDCl₃) 6.96 (1H, m), 6.80 (1H, m), 4.12 (1H, br s), 3.87 (1H, d), 3.77 (1H, q), 3.13 (1H, app td), 3.00 (1H, br d), 2.49 (1H, d), 1.96-2.10 (2H, m), 1.45 (9H, s), 1.33 (3H, d), 1.19 (3H, d).

### Example 66

### (3R)-3-Methyl-1-[(1R)-1-(2,3,5-trifluorophenyl)ethyl]piperazine (39)

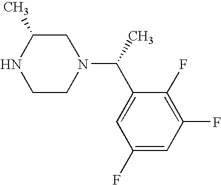

tert-Butyl carbamate 64 (30 mg, 0.084 mmol) was treated with TFA in accordance with Method O to afford the title compound as a colorless oil with spectral data consistent with that obtained for 39 as prepared in example 40.

### Example 67

### (3R)-3-Methyl-1-[(1S)-1-(2,3,5-trifluorophenyl)ethyl]piperazine (42)

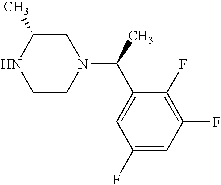

tert-Butyl carbamate 65 (27 mg, 0.075 mmol) was treated with TFA in accordance with Method O to afford the title compound as a colorless oil with spectral data consistent with that obtained for 42 as prepared in example 43.

### Example 68

### [5-(4-Methoxyphenyl)-6-methyl-7-(trifluoromethyl)pyrazolo[1,5-a]pyrimidin-3-yl][(2R)-2-methyl-4-[(1R)-1-(2,3,5-trifluorophenyl)ethyl]-1-piperazinyl]methanone (37)

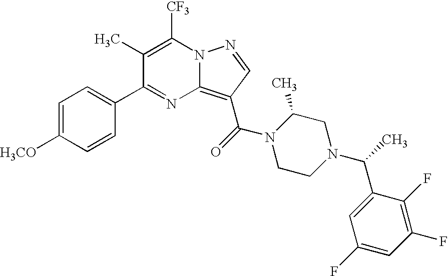

In accordance with Method H, carboxylic acid 19 (27 mg, 0.078 mmol) was converted to the analogous acid chloride via treatment with oxalyl chloride (9 μL, 0.11 mmol, d 1.455) and catalytic DMF (˜2 μL) then treated with DIPEA (19 μL, 0.11 mmol, d 0.742) and piperazine 39 (20 mg, 0.028 mmol). Column chromatography (silica gel, 20-30% EtOAc in heptanes) afforded the title compound (>80% de) as a vitreous yellow solid with spectral data consistent with that obtained for 37 as prepared in example 41.

### Example 69

### 5-(4-Methoxyphenyl)-6-methyl-7-(trifluoromethyl)pyrazolo[1,5-a]pyrimidine-3-carboxylic Acid, Methyl Ester (66)

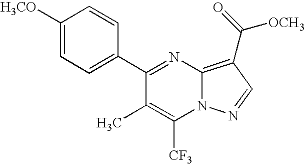

Aminopyrazole 14 (178 mg, 0.77 mmol) was treated with diketone 2 (200 mg, 0.77 mmol) using Method D to give the title compound after column chromatography (silica gel, 20-40% EtOAc in heptanes) as a white powder: LC/MS t_{R} 2.22 min; MS (ES+) m/z 334, 366, 388, 753; ¹H NMR δ_{H} (360 MHz, CDCl₃) 8.61 (1H, s), 7.62 (2H, d), 7.04 (2H, d), 3.94 (3H, s), 3.90 (3H, s), 2.59 (3H, q).

### Example 70

### [5-(4-Methoxyphenyl)-6-methyl-7-(trifluoromethyl)pyrazolo[1,5-a]pyrimidin-3-yl][(2R)-2-methyl-4-[(1S)-1-(2,3,5-trifluorophenyl)ethyl]-1-piperazinyl]methanone (40)

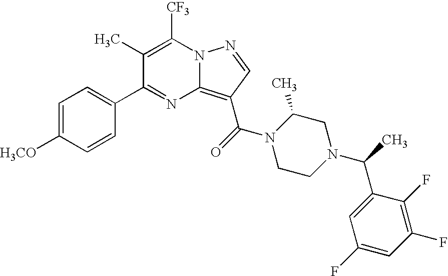

Pyrazolopyrimidine methyl ester 66 (14 mg, 0.039 mmol) was treated with piperazine 42 (15 mg, 0.058 mmol) and TiCl₄ (21 μL, 0.19 mmol, d 1.72) using Method K to afford the title compound after column chromatography (silica gel, 20-30% EtOAc in heptanes) as a vitreous yellow solid with spectral data consistent with that obtained for 40 as prepared in example 44.

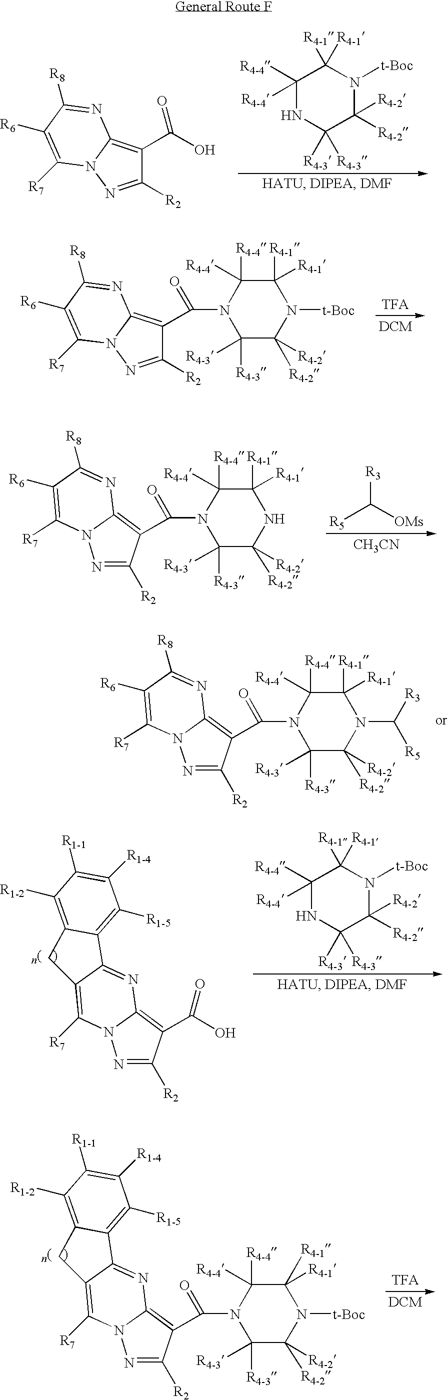

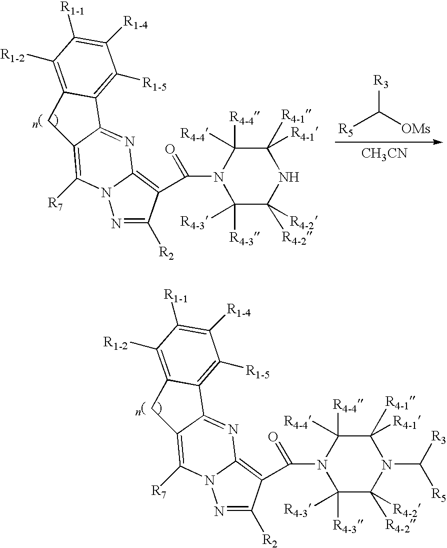

### Description of Methods Used in General Route F

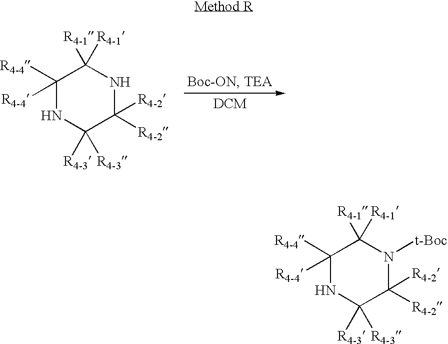

The piperazine (1 equiv) in CHCl₃ (10 vol) was treated with TEA (0.25 equiv) and Boc-ON (0.16 equiv) and stirred at rt for 16 h. The reaction was monitored by TLC (10% MeOH in DCM, stained with ninhydrin). On completion the reaction was quenched with water (10 vol) and extracted into DCM (2×10 vol). The combined organic phases were dried (Na₂SO₄), filtered and evaporated to give a residue that was purified by column chromatography (silica gel, 10% MeOH in DCM) providing the desired product.

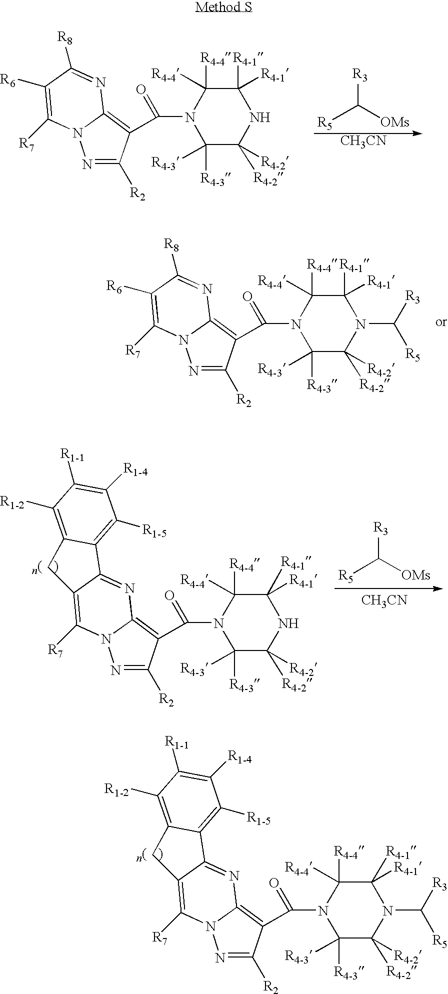

A solution of the alcohol (5 equiv) and TEA (9 equiv) in DCM (40 vol) at 0° C. was treated with MsCl (4.5 equiv) and stirred at this temperature for 1 h. Reaction progress was monitored by TLC. On completion the reaction mixture was washed with ice-cold satd NaHCO₃ (40 vol) and water (40 vol). The DCM phase was dried (Na₂SO₄) and filtered and the filtrate reduced in vacuo in the absence of a warming water bath. The mesylate thus formed was immediately dissolved in cold (0° C.) MeCN (40 vol) and treated with a separately prepared solution of the piperazine amide (1 equiv) and TEA (5 equiv) in MeCN (20 vol). The reaction was allowed to warm to rt and stirred 16 h before assessing reaction progress by LC/MS. If necessary, the reaction was warmed to 40-50° C. and stirred a further 16 h. On completion the reaction mixture was diluted with water (100 vol) and extracted with EtOAc (3×100 vol). The combined EtOAc phases were dried (MgSO₄) and filtered and the filtrate reduced in vacuo. Column chromatography (silica gel, EtOAc in heptanes or MeOH in DCM) afforded the desired product.

### Examples of Compounds Prepared by General Route F

### Example 71

### (R)-3-Methyl-1-piperazinecarboxylic Acid, 1,1-Dimethylethyl Ester (67)

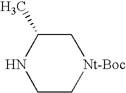

(R)-(−)-2-Methylpiperazine (1.0 g, 10.0 mmol) in CHCl₃ (10 mL) was treated with TEA (348 μL, 2.5 mmol) and Boc-ON (394 mg, 1.6 mmol) using Method R. The residue obtained was purified by column chromatography (silica gel, 10% MeOH in DCM) to give the title compound as a pale yellow oil: LC/MS t_{R} 1.86 min; MS (ES+) m/z 201.

### Example 72

### [(3R)-4-[[5-(4-Methoxyphenyl)-6-methyl-7-(trifluoromethyl)pyrazolo[1,5-a]pyrimidin-3-yl]carbonyl]-3-methyl-1-piperazinecarboxylic Acid, 1,1-Dimethylethyl Ester (68)

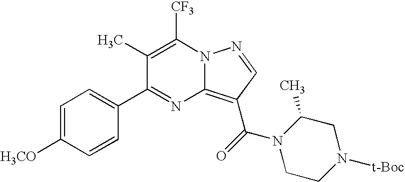

Pyrazolopyrimidine carboxylic acid 19 (2.50 g, 7.12 mmol) was treated with HATU (3.25 g, 8.54 mmol), DIPEA (1.72 mL, 9.97 mmol, d 0.747) and piperazine 67 (1.57 g, 7.83 mmol) in accordance with Method G to give the title compound as a yellow powder: LC/MS t_{R} 2.42 min; MS (ES+) m/z 534, 556.

### Example 73

### [5-(4-Methoxyphenyl)-6-methyl-7-(trifluoromethyl)pyrazolo[1,5-a]pyrimidin-3-yl][(2R)-2-methyl-1-piperazinyl]methanone (69)

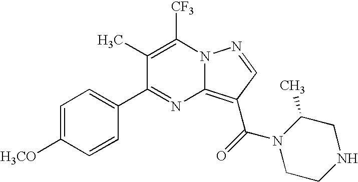

tert-Butyl carbamate 68 (2.99 g, 5.40 mmol) was treated with TFA in accordance with Method O to afford the title compound as a vitreous yellow solid: LC/MS t_{R} 1.48 min; MS (ES+) m/z 434; ¹H NMR δ_{H} (250 MHz, CDCl₃) 8.44 (1H, s), 7.52 (2H, d), 7.03 (2H, d), 4.56 (1H, br s) 4.06 (1H, br s), 3.89 (3H, s), 3.25 (1H, br m), 2.74-3.11 (4H, m), 2.55 (3H, q), 1.35 (3H, d).

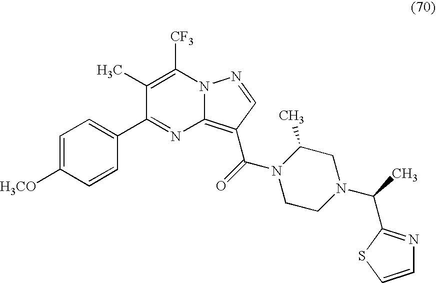

### Example 74

### (αR)-α-Methyl-2-thiazolemethanol (71)

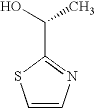

2-Acetylthiazole (0.33 mL, 3.15 mmol, d 1.227) was treated with (+)-DIP-Cl (2.22 g, 6.92 mmol) in accordance with Method P to give the title compound after column chromatography (silica gel, 10-60% EtOAc in heptanes) as a brown viscous oil: LC/MS t_{R} 0.94 min; MS (ES+) m/z 112, 130; ¹H NMR δ_{H} (250 MHz, CDCl₃) 7.72 (1H, d), 7.30 (1H, d), 5.17 (1H, q), 3.37 (1H, br s), 1.65 (3H, d).

### Example 75

### [5-(4-Methoxyphenyl)-6-methyl-7-(trifluoromethyl)pyrazolo[1,5-a]pyrimidin-3-yl][(2R)-4-[(1S)-1-(2-thiazolyl)ethyl]-2-methyl-1-piperazinyl]methanone (70)

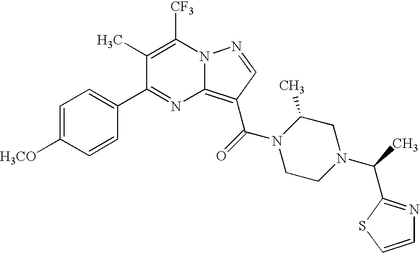

In accordance with Method S, alcohol 71 (45 mg, 0.35 mmol) was treated with MsCl (24 μL, 0.31 mmol, d 1.48) and TEA (87 μL, 0.62 mmol, d 0.726) to fashion the corresponding mesylate. Piperazine 69 (30 mg, 0.069 mmol) was treated immediately with this mesylate and TEA (48 μL, 0.35 mmol, d 0.726). Column chromatography (silica gel, 40-60% EtOAc in heptanes) followed by extraction of a methanolic solution (10 mL) of the purified compound with heptane (3×10 mL) and reduction of the MeOH in vacuo afforded the title compound as a vitreous yellow solid: LC/MS t_{R} 1.96 min; MS (ES+) m/z 545; ¹H NMR δ_{H} (250 MHz, CDCl₃) 8.44 (1H, s), 7.70 (1H, d), 7.52 (2H, d), 7.29 (1H, m), 7.03 (2H, d), 4.16-4.89 (1H, br s), 4.07 (1H, m), 3.91 (3H, s), 3.42 (1H, br s), 3.17 (1H, br d), 2.55 (3H, q), 2.42-2.91 (4H, m), 1.45 (3H, d), 1.42 (3H, d).

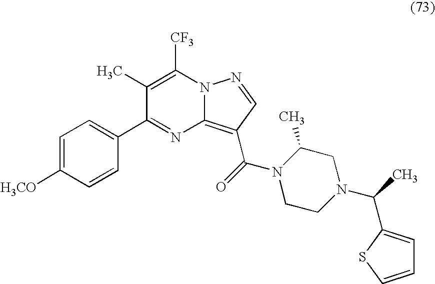

### Example 76

### (αR)-α-Methyl-2-thiophenemethanol (74)

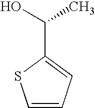

2-Acetylthiophene (0.34 mL, 3.17 mmol, d 1.168) was treated with (+)-DIP-Cl (2.24 g, 6.97 mmol) in accordance with Method P to give the title compound admixed with α-pinene after column chromatography (silica gel, 5-40% EtOAc in heptanes) as a colorless oil: LC/MS t_{R} 1.37 min; MS (ES+) m/z III; ¹H NMR δ_{H} (250 MHz, CDCl₃) 7.25 (1H, dd), 6.91-7.03 (2H, m), 5.15 (1H, q), 1.62 (3H, d).

### Example 77

### [5-(4-Methoxyphenyl)-6-methyl-7-(trifluoromethyl)pyrazolo[1,5-a]pyrimidin-3-yl][(2R)-4-[(is)-1-(2-thienyl)ethyl]-2-methyl-1-piperazinyl]methanone (73)

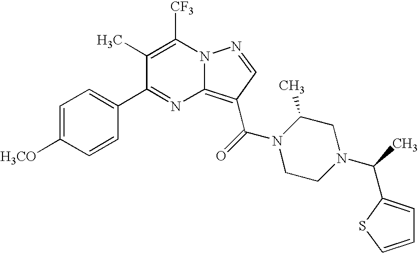

In accordance with Method S, alcohol 74 (89 mg, 0.35 mmol, ca. 53% purity) was treated with MsCl (24 μL, 0.31 mmol, d 1.48) and TEA (87 μL, 0.62 mmol, d 0.726) to fashion the corresponding mesylate. Piperazine 69 (30 mg, 0.069 mmol) was treated immediately with this mesylate and TEA (48 μL, 0.35 mmol, d 0.726). Column chromatography (silica gel, 30-50% EtOAc in heptanes) followed by extraction of a methanolic solution (10 mL) of the purified compound with heptane (3×10 mL) and reduction of the MeOH in vacuo afforded the title compound as a vitreous yellow solid: LC/MS t_{R} 1.76 min; MS (ES+) m/z 544; ¹H NMR δ_{H} (250 MHz, CDCl₃) 8.45 (1H, s), 7.54 (2H, d), 7.24 (1H, t), 7.05 (2H, d), 6.96 (1H, m), 6.87 (1H, s), 4.09-5.04 (1H, br s), 3.94 (3H, s), 3.78-4.08 (2H, obs m), 3.39 (1H, br s), 2.56 (3H, q), 2.23-2.93 (4H, m), 1.42 (3H, d), 1.40 (3H, d).

### Example 78

### [(2R)-4-[(1S)-1-(2-Furanyl)ethyl]-2-methyl-1-piperazinyl][5-(4-methoxyphenyl)-6-methyl-7-(trifluoromethyl)pyrazolo[1,5-a]pyrimidin-3-yl]methanone (75)

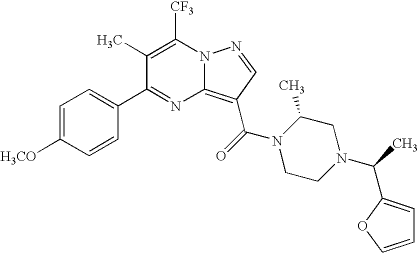

In accordance with Method S, (R)-(+)-1-(2-furanyl)ethanol (39 mg, 0.35 mmol) was treated with MsCl (24 μL, 0.31 mmol, d 1.48) and TEA (87 μL, 0.62 mmol, d 0.726) to fashion the corresponding mesylate. Piperazine 69 (30 mg, 0.069 mmol) was treated immediately with this mesylate and TEA (48 μL, 0.35 mmol, d 0.726). Column chromatography (silica gel, 30-50% EtOAc in heptanes) afforded the title compound as a vitreous yellow solid: LC/MS t_{R} 1.58 min; MS (ES+) m/z 528; ¹H NMR δ_{H} (360 MHz, CDCl₃) 8.43 (1H, s), 7.54 (2H, m), 7.35 (1H, m), 7.06 (2H, d), 6.32 (1H, s), 6.12 (1H, m), 4.39-4.78 (1H, br s), 3.94 (3H, s), 3.80 (1H, m), 3.38 (1H, br s), 2.57 (3H, q), 2.37-2.88 (4H, m), 2.24 (1H, t), 1.41 (3H, d), 1.39 (3H, d).

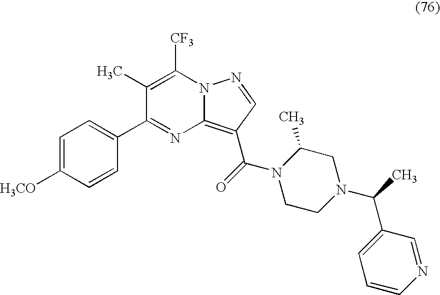

### Example 79

### (αR)-α-Methyl-3-pyridinemethanol (77)

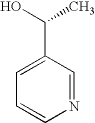

3-Acetylpyridine (0.36 mL, 3.31 mmol, d 1.106) was treated with (+)-DIP-Cl (2.34 g, 7.28 mmol) in accordance with Method P (alternative procedure) to give the title compound after column chromatography (silica gel, 80-100% EtOAc in heptanes) as a colorless viscous oil: LC/MS t_{R} 0.28 min; MS (ES+) m/z 124; ¹H NMR δ_{H} (250 MHz, CDCl₃) 8.57 (1H, d), 8.48 (1H, dd), 7.74 (1H, app dt), 7.28 (1H, dd), 4.96 (1H, q), 2.68 (1H, br s), 1.53 (3H, d).

### Example 80

### [5-(4-Methoxyphenyl)-6-methyl-7-(trifluoromethyl)pyrazolo[1,5-a]pyrimidin-3-yl][(2R)-4-[(1S)-2-methyl-1-piperazinyl-1-(3-pyridinyl)ethyl]methanone (76)

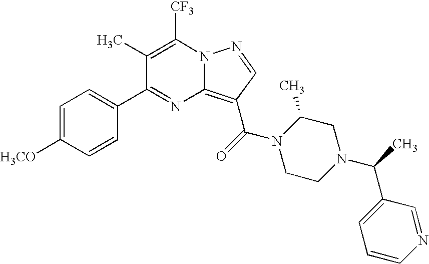

In accordance with Method S, alcohol 77 (43 mg, 0.35 mmol) was treated with MsCl (24 μL, 0.31 mmol, d 1.48) and TEA (87 μL, 0.62 mmol, d 0.726) to fashion the corresponding mesylate. Piperazine 69 (30 mg, 0.069 mmol) was treated immediately with this mesylate and TEA (48 μL, 0.35 mmol, d 0.726). Column chromatography (silica gel, 0-2% MeOH in DCM) afforded the title compound as a vitreous yellow solid: LC/MS t_{R} 1.54 min; MS (ES+) m/z 539, 561; ¹H NMR δ_{H} (250 MHz, CDCl₃) 8.55 (1H, s), 8.51 (1H, m), 8.42 (1H, s), 7.66 (1H, d), 7.50 (2H, d), 7.25 (1H, m), 7.04 (2H, d), 4.03-4.95 (1H, br s), 3.93 (3H, s), 3.83 (1H, br s), 3.21-3.53 (2H, m), 2.99 (1H, br s), 2.54 (3H, q), 2.46-2.60 (1H, obs m), 2.28 (1H, dd), 2.18 (1H, m), 1.35 (3H, d), 1.32 (3H, d).

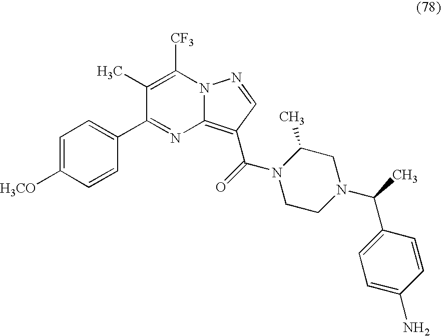

### Example 81

### N-(4-Acetylphenyl)carbamic Acid, 1,1-Dimethyl Ester (79)

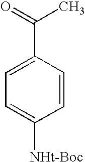

A solution of p-aminoacetophenone (1.0 g, 7.40 mmol) in THF (5 mL) was treated with a solution of di-tert-butyl dicarbonate (1.78 g, 8.14 mmol) in THF (5 mL) and DMAP (10 mg) and stirred at rt for 16 h before warming to 60° C. and stirring 2 h. A further portion of di-tert-butyl dicarbonate (0.67 g, 3.07 mmol) was added at this juncture and the reaction was stirred at 60° C. a further 16 h before removing the solvent in vacuo. Column chromatography (silica gel, 10-30% EtOAc in heptanes) followed by recrystallization (heptane) afforded the title compound as a white solid: LC/MS t_{R} 2.02 min; MS (ES+) m/z 236; ¹H NMR δ_{H} (360 MHz, CDCl₃) 7.92 (2H, d), 7.46 (2H, d), 6.73 (1H, br s), 2.57 (3H, s), 1.54 (9H, s).

### Example 82

### N-[4-[(1R)-1-Hydroxyethyl]phenyl]carbamic Acid, 1,1-Dimethyl Ester (80)

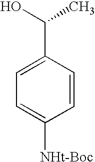

Acetophenone 79 (0.86 g, 3.66 mmol) was treated with (+)-DIP-Cl (1.29 g, 4.02 mmol) in accordance with Method P to give the title compound after column chromatography (silica gel, 0-30% EtOAc in heptanes) as an off-white waxy solid: LC/MS t_{R} 1.73 min; MS (ES+) m/z 164, 220, ¹H NMR δ_{H} (250 MHz, CDCl₃) 7.34 (2H, d), 7.30 (2H, d), 6.47 (1H, br s), 4.86 (1H, app qd), 1.75 (1H, br d), 1.52 (9H, s), 1.48 (3H, d).

### Example 83

### N-[4-[(1S)-1-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]ethyl]phenyl]carbamic Acid, 1,1-Dimethylethyl Ester (81)

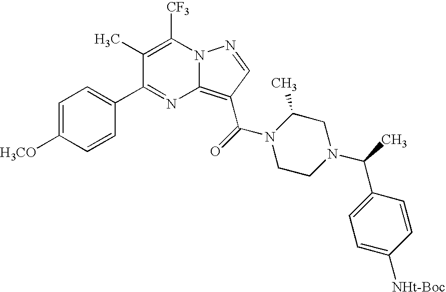

In accordance with Method S, alcohol 80 (0.37 g, 1.56 mmol) was treated with MsCl (109 μL, 1.40 mmol, d 1.48) and TEA (0.39 mL, 2.81 mmol, d 0.726) to fashion the corresponding mesylate. Piperazine 69 (0.14 g, 0.31 mmol) was treated immediately with this mesylate and TEA (0.22 mL, 1.56 mmol, d 0.726). Column chromatography (silica gel, 0-50% EtOAc in heptanes) afforded the title compound as a vitreous yellow solid: LC/MS t_{R} 1.77 min; MS (ES+) m/z 653; ¹H NMR δ_{H} (360 MHz, CDCl₃) 8.41 (1H, s), 7.50 (2H, d), 7.30 (2H, d), 7.23 (2H, m), 7.03 (2H, d), 6.46 (1H, s), 3.98-4.59 (1H, br s), 3.93 (3H, s), 3.50 (1H, d), 3.14-3.43 (2H, m), 2.60-3.04 (1H, br m), 2.54 (3H, q), 2.05-2.60 (3H, m), 1.30 (3H, d), 1.27 (3H, d).

### Example 84

### [(2R)-4-[(1S)-1-(4-Aminophenyl)ethyl]-2-methyl-1-piperazinyl][5-(4-methoxyphenyl)-6-methyl-7-(trifluoromethyl)pyrazolo[1,5-a]pyrimidin-3-yl]methanone (78)

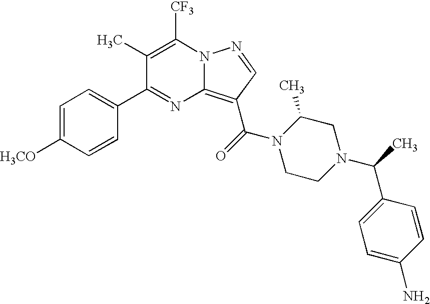

tert-Butyl carbamate 81 (90 mg, 0.14 mmol) was treated with TFA employing the procedure of Method O to afford the title compound after column chromatography (silica gel, 0-2% MeOH in DCM) as a vitreous yellow solid: LC/MS t_{R} 1.52 min; MS (ES+) m/z 434, 553, 575; ¹H NMR δ_{H} (250 MHz, CDCl₃) 8.41 (1H, s), 7.50 (2H, d), 7.10 (2H, d), 7.03 (2H, d), 6.63 (2H, d), 3.97-4.78 (1H, br s), 3.90 (3H, s), 3.81-3.96 (1H, obs br s), 3.62 (2H, br s), 3.30 (2H, br s), 2.59-3.03 (1H, br m), 2.55 (3H, q), 2.36-2.58 (1H, br s), 1.97-2.35 (2H, m), 1.21-1.42 (6H, m).

### Example 85

### N-[4-[(1S)-1-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]ethyl]phenyl]acetamide (82)

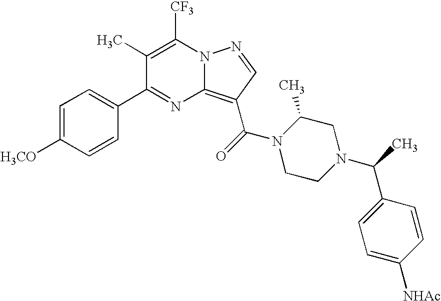

A solution of aniline 78 (20 mg, 0.036 mmol) and TEA (10 μL, 0.072 mmol, d 0.726) in DCM (1.5 mL) was cooled to 0° C. and treated with acetyl chloride (2.8 μL, 0.040 mmol, d 1.104). After stirring at rt for 1 h, the reaction mixture was diluted with DCM (10 mL) and satd NaHCO₃ (10 mL) and the two phases separated. The organic phase was washed with water (2×10 mL) and brine (10 mL), dried (Na₂SO₄), filtered and reduced in vacuo to afford the title compound as a vitreous yellow solid: LC/MS t_{R} 1.50 min; MS (ES+) m/z 595; ¹H NMR δ_{H} (250 MHz, CDCl₃) 8.40 (1H, s), 7.56 (1H, br s), 7.49 (2H, d), 7.43 (2H, d), 7.23 (2H, dd), 7.03 (2H, d), 4.00-4.94 (1H, br s), 3.92 (3H, s), 3.32 (2H, m), 2.59-3.12 (1H, br m), 2.53 (3H, q), 2.01-2.58 (4H, m), 2.15 (3H, s), 1.19-1.42 (6H, m).

### Example 86

### N-[4-[(1S)-1-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]ethyl]phenyl]urea (83)

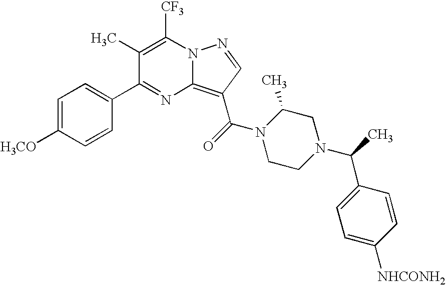

A solution of aniline 78 (20 mg, 0.036 mmol) in AcOH (0.5 mL) and water (0.5 mL) was treated with potassium cyanate (18 mg, 0.22 mmol) and stirred at rt for 16 h. Reaction progress was monitored by LC/MS. On completion the reaction was diluted with water (4 mL) and extracted into EtOAc (3×5 mL). The combined EtOAc phases were washed with satd NaHCO₃ (2×10 mL), dried (Na₂SO₄) and filtered and the filtrate reduced in vacuo. Column chromatography (silica gel, 0-2% MeOH in EtOAc) afforded the title compound as a vitreous yellow solid: LC/MS t_{R} 1.46 min; MS (ES+) m/z 434, 596, 618; ¹H NMR δ_{H} (360 MHz, CDCl₃) 8.37 (1H, s), 7.58-7.83 (1H, br s), 7.48 (2H, d), 7.22 (2H, d), 7.18 (2H, m), 7.02 (2H, d), 4.96 (2H, s), 4.19-4.80 (1H, br s), 3.90 (3H, s), 3.75 (1H, t), 2.89-3.45 (2H, m), 2.59-2.81 (1H, m), 2.52 (3H, q), 2.50 (1H, obs br s), 2.07-2.35 (2H, m), 1.21-1.39 (6H, m).

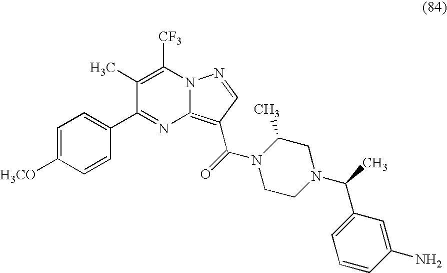

### Example 87

### N-(3-Acetylphenyl)carbamic Acid, 1,1-Dimethyl Ester (85)

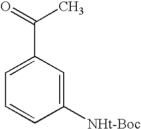

A solution of 3-aminoacetophenone (1.0 g, 7.40 mmol) in THF (5 mL) was treated with a solution of di-tert-butyl dicarbonate (2.42 g, 11.1 mmol) in THF (5 mL) and DMAP (10 mg) and stirred at 60° C. for 16 h. After cooling to rt, the solvent was removed in vacuo. Column chromatography (silica gel, 10-30% EtOAc in heptanes) followed by recrystallization (heptane) gave a 1:1 mixture of bis-Boc protected and mono-Boc protected material. This mixture was taken into 1:1 THF/EtOH (2 mL) and treated with 6 M NaOH (3 mL). After 16 h stirring at rt, the reaction mixture was diluted with water (10 mL) and extracted with MTBE (3×10 mL). The combined organic phases were washed with water (5 mL) and brine (5 mL), dried (MgSO₄), filtered and reduced in vacuo to afford the title compound as a white solid: LC/MS t_{R} 1.96 min; MS (ES+) m/z 180, 258; ¹H NMR δ_{H} (250 MHz, CDCl₃) 7.93 (1H, t), 7.60-7.70 (2H, m), 7.39 (1H, t), 6.60 (1H, br s), 2.61 (3H, s), 1.54 (9H, s).

### Example 88

### N-[3-[(1R)-1-Hydroxyethyl]phenyl]carbamic Acid, 1,1-Dimethyl Ester (86)

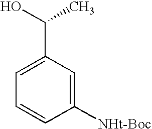

Acetophenone 85 (0.34 g, 1.45 mmol) was treated with (+)-DIP-Cl (0.51 g, 1.60 mmol) in accordance with Method P to give the title compound after column chromatography (silica gel, 0-30% EtOAc in heptanes) as an off-white waxy solid: LC/MS t_{R} 1.75 min; MS (ES+) m/z 164, 220; ¹H NMR δ_{H} (250 MHz, CDCl₃) 7.43 (1H, br s), 7.28 (1H, t), 7.22 (1H, app dt), 7.06 (1H, app dt), 6.50 (1H, br s), 4.88 (1H, q), 2.05 (1H, s), 1.53 (9H, s), 1.49 (3H, d).

### Example 89

### N-[3-[(1S)-1-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]ethyl]phenyl]carbamic Acid, 1,1-Dimethylethyl Ester (87)

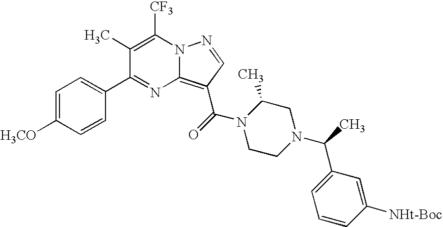

In accordance with Method S, alcohol 86 (0.26 g, 1.10 mmol) was treated with MsCl (77 μL, 0.99 mmol, d 1.48) and TEA (0.28 mL, 1.97 mmol, d 0.726) to fashion the corresponding mesylate. Piperazine 69 (0.16 g, 0.37 mmol) was treated immediately with this mesylate and TEA (0.15 mL, 1.10 mmol, d 0.726). Column chromatography (silica gel, 0-40% EtOAc in heptanes) afforded the title compound as a vitreous yellow solid: LC/MS t_{R} 1.81 min; MS (ES+) m/z 653; ¹H NMR δ_{H} (250 MHz, CDCl₃) 8.41 (1H, s), 7.51 (2H, d), 7.17-7.34 (3H, m), 7.03 (2H, d), 7.00 (1H, obs m), 6.48 (1H, s), 3.98-4.74 (1H, br s), 3.92 (3H, s), 3.28 (1H m), 3.19-3.67 (1H, br s), 2.63-3.18 (1H, br m), 2.54 (3H, q), 2.52 (1H, obs br s), 2.01-2.39 (2H, m), 1.33 (3H, d), 1.28 (3H, d).

### Example 90

### [(2R)-4-[(1S)-1-(3-Aminophenyl)ethyl]-2-methyl-1-piperazinyl][5-(4-methoxyphenyl)-6-methyl-7-(trifluoromethyl)pyrazolo[1,5-a]pyrimidin-3-yl]methanone (84)

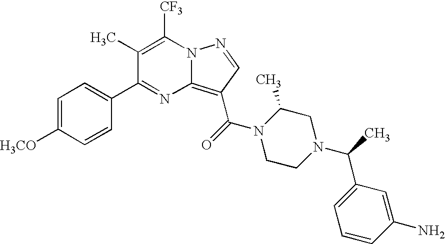

tert-Butyl carbamate 87 (110 mg, 0.17 mmol) was treated with TFA employing the procedure of Method O to afford the title compound as a vitreous yellow solid: LC/MS t_{R} 1.52 min; MS (ES+) m/z 553, 575; ¹H NMR δ_{H} (250 MHz, CDCl₃) 8.42 (1H, s), 7.51 (2H, d), 7.08 (1H, t), 7.03 (2H, d), 6.70 (1H, d), 6.65 (1H, s), 6.56 (1H, dd), 3.97-5.02 (1H, br s), 3.92 (3H, s), 3.63 (2H, br s), 3.39 (1H, br s), 3.21 (1H, m), 3.01 (1H, br s), 2.54 (3H, q), 2.46-2.63 (1H, obs br s), 2.02-2.23 (2H, m), 1.33 (3H, d), 1.27 (3H, d).

### Example 91

### N-[3-[(1S)-1-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]ethyl]phenyl]acetamide (88)

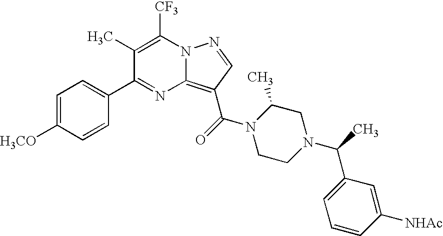

A solution of aniline 84 (20 mg, 0.036 mmol) and TEA (10 μL, 0.072 mmol, d 0.726) in DCM (1.5 mL) was cooled to 0° C. and treated with acetyl chloride (2.8 μL, 0.040 mmol, d 1.104). After stirring at rt for 1 h, the reaction mixture was diluted with DCM (10 mL) and satd NaHCO₃ (10 mL) and the two phases separated. The organic phase was washed with water (2×10 mL) and brine (10 mL), dried (Na₂SO₄), filtered and reduced in vacuo to afford the title compound as a vitreous yellow solid: LC/MS t_{R} 1.55 min; MS (ES+) m/z 595; ¹H NMR δ_{H} (250 MHz, CDCl₃) 8.40 (1H, s), 7.50 (2H, d), 7.32-7.50 (3H, m), 7.24 (1H, t), 7.06 (1H, obs m), 7.03 (2H, d), 4.01-5.08 (1H, br s), 3.92 (3H, s), 3.84 (1H, obs br s), 3.35 (1H, br s), 3.28 (1H, m), 2.72-3.12 (1H, br m), 2.54 (3H, q), 2.50 (1H, obs br s), 2.16 (3H, s), 1.97-2.24 (2H, obs m), 1.33 (3H, d), 1.27 (3H, d).

### Example 92

### N-[3-[(1S)-1-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]ethyl]phenyl]urea (89)

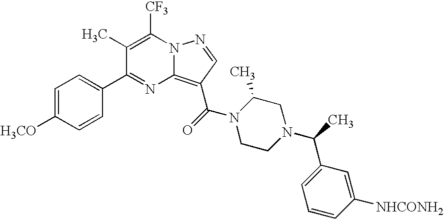

A solution of aniline 84 (10 mg, 0.018 mmol) in AcOH (0.5 mL) and water (0.5 mL) was treated with potassium cyanate (8.8 mg, 0.11 mmol) and stirred at rt for 16 h. Reaction progress was monitored by LC/MS. On completion the reaction was diluted with water (4 mL) and extracted into EtOAc (3×5 mL). The combined EtOAc phases were washed with satd NaHCO₃ (2×10 mL), dried (Na₂SO₄) and filtered and the filtrate reduced in vacuo. Column chromatography (silica gel, 60-100% EtOAc in heptanes) afforded the title compound as a vitreous yellow solid: LC/MS t_{R} 1.50 min; MS (ES+) m/z 596, 618; ¹H NMR δ_{H} (250 MHz, CDCl₃) 8.40 (1H, s), 7.49 (2H, d), 7.12-7.37 (3H, m), 7.03 (2H, d), 7.01 (1H, obs m), 6.86 (1H, s), 4.71 (2H, s), 3.99-4.61 (1H, br s), 3.92 (3H, s), 2.92-3.67 (2H, br m), 2.54 (3H, q), 2.51 (1H, obs br s), 2.06-2.28 (2H, m), 1.28-1.36 (6H, m).

### Example 93

### N-[3-[(1S)-1-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]ethyl]phenyl]methanesulfonamide (90)

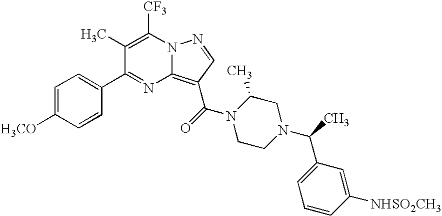

A solution of aniline 84 (30 mg, 0.054 mmol) and TEA (15.1 μL, 0.11 mmol, d 0.726) in DCM (1.5 mL) was cooled to 0° C. and treated with MsCl (4.4 μL, 0.057 mmol, d 1.48). After stirring at rt for 1 h, the reaction mixture was diluted with DCM (10 mL) and satd NaHCO₃ (10 mL) and the two phases separated. The organic phase was washed with water (2×10 mL) and brine (10 mL), dried (Na₂SO₄), filtered and reduced in vacuo. Column chromatography (40-70% EtOAc in heptanes) afforded the title compound as a vitreous yellow solid: LC/MS t_{R} 1.58 min; MS (ES+) m/z 631; ¹H NMR δ_{H} (250 MHz, CDCl₃) 8.41 (1H, s), 7.51 (2H, d), 7.27 (1H, obs t), 7.21 (1H, s), 7.12 (1H, s), 7.08 (2H, m), 7.03 (2H, d), 4.18-5.14 (1H, br s), 3.92 (3H, s), 3.50-4.17 (1H, br s), 3.32 (1H, br s), 3.30 (1H, m), 2.97 (3H, s), 2.94 (1H, obs br s), 2.54 (3H, q), 2.49 (1H, obs br s), 2.05-2.26 (2H, m), 1.32 (3H, d), 1.27 (3H, d).

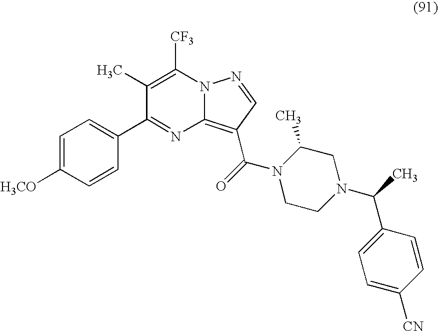

### Example 94

### 4-[(1R)-1-Hydroxyethyl]benzonitrile (92)

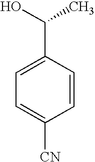

4-Acetylbenzonitrile (0.40 g, 2.76 mmol) was treated with (+)-DIP-Cl (1.94 g, 6.06 mmol) in accordance with Method P to give the title compound after column chromatography (silica gel, 0-50% EtOAc in heptanes) as a pale orange oil: ¹H NMR δ_{H} (250 MHz, CDCl₃) 7.64 (2H, d), 7.49 (2H, d), 4.97 (1H, q), 2.05 (1H, br s), 1.50 (3H, d).

### Example 95

### 4-[(1S)-1-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]ethyl]benzonitrile (91)

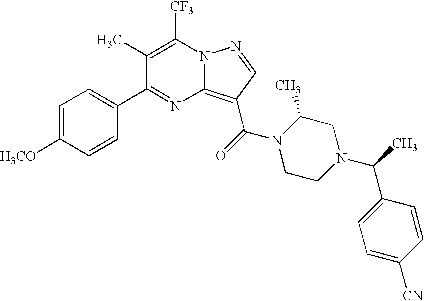

In accordance with Method S, alcohol 92 (60 mg, 0.35 mmol, ca. 85% purity) was treated with MsCl (24 μL, 0.31 mmol, d 1.48) and TEA (87 μL, 0.62 mmol, d 0.726) to fashion the corresponding mesylate. Piperazine 69 (30 mg, 0.069 mmol) was treated immediately with this mesylate and TEA (48 μL, 0.35 mmol, d 0.726). Column chromatography (silica gel, 30-50% EtOAc in heptanes) afforded the title compound as a vitreous yellow solid: LC/MS t_{R} 1.79 min; MS (ES+) m/z 563; ¹H NMR δ_{H} (250 MHz, CDCl₃) 8.41 (1H, s), 7.60 (2H, d), 7.50 (2H, d), 7.44 (2H, d), 7.03 (2H, d), 3.92 (3H, s), 3.71-5.14 (2H, br s), 3.36 (1H, m), 3.35 (1H, obs br s), 2.98 (1H, br s), 2.54 (3H, q), 2.32-2.60 (1H, obs m), 2.10-2.32 (2H, m), 1.31 (3H, d), 1.28 (3H, d).

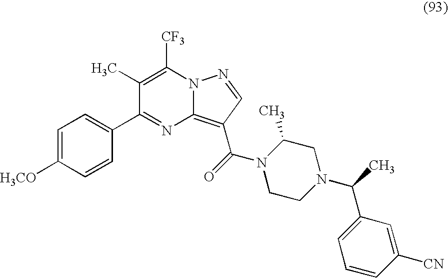

### Example 96

### 3-[(1R)-1-Hydroxyethyl]benzonitrile (94)

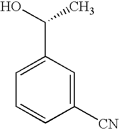

3-Acetylbenzonitrile (0.40 g, 2.76 mmol) was treated with (+)-DIP-Cl (1.94 g, 6.06 mmol) in accordance with Method P to give the title compound after column chromatography (silica gel, 0-50% EtOAc in heptanes) as a white waxy solid: ¹H NMR δ_{H} (250 MHz, CDCl₃) 7.69 (1H, t), 7.62 (1H, app dt), 7.56 (1H, app dt), 7.46 (1H, t), 4.95 (1H, q), 1.96 (1H, br s), 1.50 (3H, d).

### Example 97

### 3-[(1S)-1-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]ethyl]benzonitrile (93)

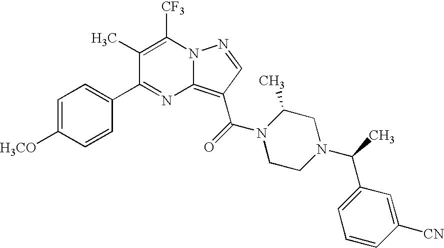

In accordance with Method S, alcohol 94 (64 mg, 0.35 mmol, ca. 80% purity) was treated with MsCl (24 μL, 0.31 mmol, d 1.48) and TEA (87 μL, 0.62 mmol, d 0.726) to fashion the corresponding mesylate. Piperazine 69 (30 mg, 0.069 mmol) was treated immediately with this mesylate and TEA (48 μL, 0.35 mmol, d 0.726). Column chromatography (silica gel, 30-50% EtOAc in heptanes) afforded the title compound as a vitreous yellow solid: LC/MS t_{R} 1.79 min; MS (ES+) m/z 563, 585; ¹H NMR δ_{H} (250 MHz, CDCl₃) 8.41 (1H, s), 7.31-7.72 (6H, m), 7.03 (2H, d), 3.92 (3H, s), 3.59-4.96 (2H, br s), 3.36 (1H m), 3.34 (1H, obs br s), 2.93 (1H, br s), 2.54 (3H, q), 2.33-2.58 (1H, obs m), 2.06-2.30 (2H, m), 1.33 (3H, d), 1.27 (3H, d).

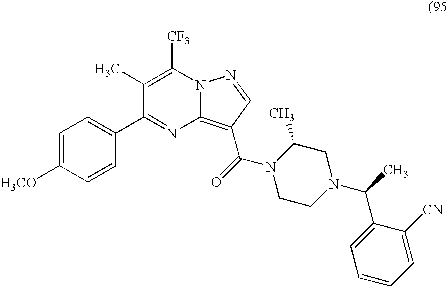

### Example 98

### 2-[(1R)-1-Hydroxyethyl]benzonitrile (96)

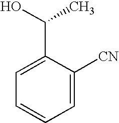

2-Acetylbenzonitrile (0.40 g, 2.76 mmol) was treated with (+)-DIP-Cl (1.94 g, 6.06 mmol) in accordance with Method P to give the title compound admixed with α-pinene after column chromatography (silica gel, 0-50% EtOAc in heptanes) as a colorless oil: ¹H NMR δ_{H} (250 MHz, CDCl₃) 7.56-7.67 (2H, m), 7.38 (1H, dd), 7.33 (1H, d), 5.31 (1H, q), 1.56 (3H, d).

### Example 99

### 2-[(1S)-1-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]ethyl]benzonitrile (95)

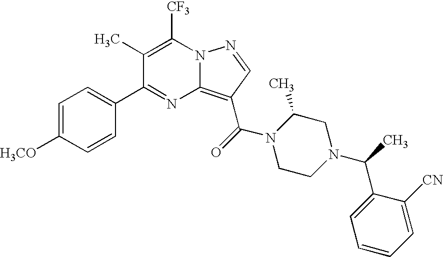

In accordance with Method S, alcohol 96 (102 mg, 0.35 mmol, ca. 50% purity) was treated with MsCl (24 μL, 0.31 mmol, d 1.48) and TEA (87 μL, 0.62 mmol, d 0.726) to fashion the corresponding mesylate. Piperazine 69 (30 mg, 0.069 mmol) was treated immediately with this mesylate and TEA (48 μL, 0.35 mmol, d 0.726). Column chromatography (silica gel, 30-50% EtOAc in heptanes) afforded the title compound as a vitreous yellow solid: LC/MS t_{R} 2.20 min; MS (ES+) m/z 563, 585; ¹H NMR δ_{H} (250 MHz, CDCl₃) 8.43 (1H, s), 7.41-7.68 (5H, m), 7.34 (1H, m), 7.06 (2H, d), 3.92 (3H, s), 3.85-5.04 (2H, br s), 3.79 (1H, m), 3.37 (1H, br s), 3.03 (1H, br d), 2.55 (3H, q), 2.18-2.62 (3H, m), 1.37 (3H, d), 1.32 (3H, d).

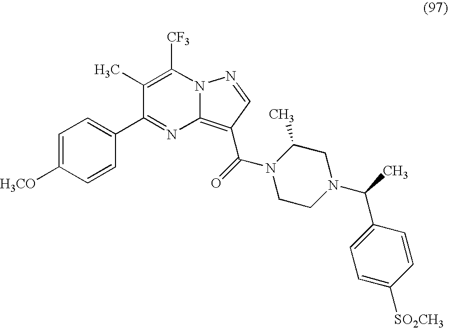

### Example 100

### (αR)-α-Methyl-4-(methylsulfonyl)benzenemethanol (98)

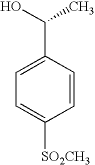

4-(Methylsulfonyl)acetophenone (0.50 g, 2.52 mmol) was treated with (+)-DIP-Cl (1.78 g, 5.55 mmol) in accordance with Method P to give the title compound after column chromatography (silica gel, 0-60% EtOAc in heptanes) as a white solid: LC/MS t_{R} 1.08 min; MS (ES+) m/z 183, 223; ¹H NMR δ_{H} (250 MHz, CDCl₃) 7.91 (2H, d), 7.58 (2H, d), 5.02 (1H, q), 3.05 (3H, s), 2.05 (1H, br s), 1.53 (3H, d).

### Example 101

### [5-(4-Methoxyphenyl)-6-methyl-7-(trifluoromethyl)pyrazolo[1,5-a]pyrimidin-3-yl][(2R)-2-methyl-4-[(1S)-1-[4-(methylsulfonyl)phenyl]ethyl]-1-piperazinyl]methanone (97)

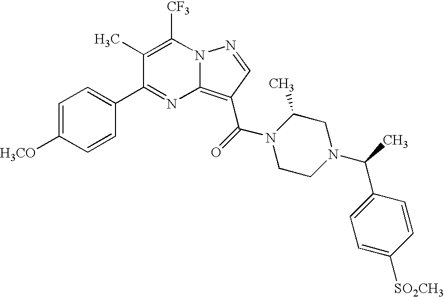

In accordance with Method S, alcohol 98 (200 mg, 0.99 mmol) was treated with MsCl (70 μL, 0.90 mmol, d 1.48) and TEA (0.25 mL, 1.80 mmol, d 0.726) to fashion the corresponding mesylate. Piperazine 69 (140 mg, 0.33 mmol) was treated immediately with this mesylate and TEA (0.14 mL, 0.99 mmol, d 0.726). Column chromatography (silica gel, 0-70% EtOAc in heptanes) afforded the title compound as a vitreous yellow solid: LC/MS t_{R} 1.65 min; MS (ES+) m/z 616; ¹H NMR δ_{H} (250 MHz, CDCl₃) 8.43 (1H, s), 7.89 (2H, d), 7.54 (2H, d), 7.50 (2H, d), 7.03 (2H, d), 3.93 (3H, s), 3.68-5.11 (2H, br s), 3.40 (1H, m), 3.40 (1H, obs br s), 3.07 (3H, s), 3.03 (1H, br s), 2.54 (3H, q), 2.34-2.59 (1H, br s), 2.10-2.29 (2H, m), 1.33 (6H, 2 d).

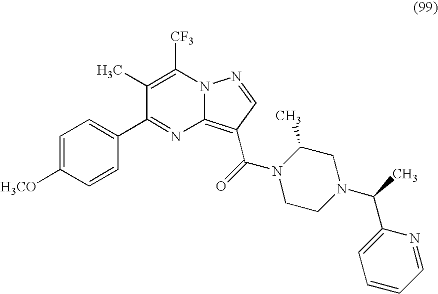

### Example 102

### (αR)-α-Methyl-2-pyridinemethanol (100)

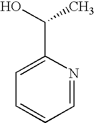

2-Acetylpyridine (0.37 mL, 3.31 mmol, d 1.08) was treated with (+)-DIP-Cl (2.34 g, 7.28 mmol) in accordance with Method P (alternative procedure) to give the title compound after column chromatography (silica gel, 80% EtOAc in heptanes) as a colorless viscous oil: LC/MS t_{R} 0.26 min; MS (ES+) m/z 124; ¹H NMR δ_{H} (250 MHz, CDCl₃) 8.55 (1H, d), 7.71 (1H, app td), 7.29 (1H, d), 7.22 (1H, dd), 4.91 (1H, q), 1.52 (3H, d).

### Example 103

### [5-(4-Methoxyphenyl)-6-methyl-7-(trifluoromethyl)pyrazolo[1,5-a]pyrimidin-3-yl][(2R)-2-methyl-4-[(1S)-1-(2-pyridinyl)ethyl]-1-piperazinyl]methanone (99)

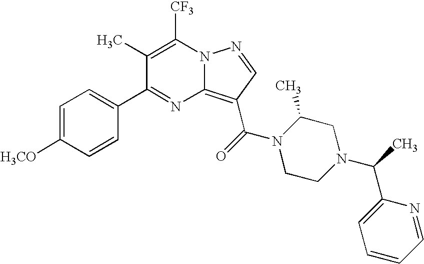

In accordance with Method S, alcohol 100 (43 mg, 0.35 mmol) was treated with MsCl (24 μL, 0.31 mmol, d 1.48) and TEA (87 μL, 0.62 mmol, d 0.726) to fashion the corresponding mesylate. Piperazine 69 (30 mg, 0.069 mmol) was treated immediately with this mesylate and TEA (48 μL, 0.35 mmol, d 0.726). Column chromatography (silica gel, 0-2% MeOH in DCM) afforded the title compound as a vitreous yellow solid: LC/MS t_{R} 1.58 min; MS (ES+) m/z 539, 561; ¹H NMR δ_{H} (250 MHz, CDCl₃) 8.52 (1H, m), 8.40 (1H, s), 7.65 (1H, t), 7.51 (2H, d), 7.45 (1H, t), 7.15 (1H, dd), 7.03 (2H, d), 3.91 (3H, s), 3.68-5.06 (2H, br s), 3.59 (1H, m), 3.35 (1H, br s), 2.59-3.11 (1H, br m), 2.54 (3H, q), 2.18-2.58 (3H, m), 1.31-1.41 (6H, m).

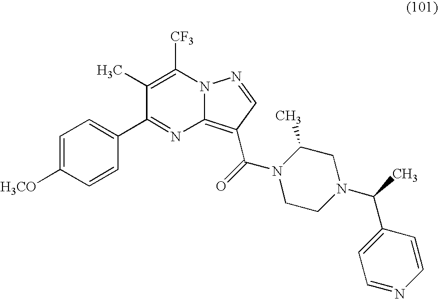

### Example 104

### (αR)-α-Methyl-4-pyridinemethanol (102)

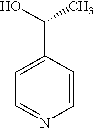

4-Acetylpyridine (0.37 mL, 3.31 mmol, d 1.09) was treated with (+)-DIP-Cl (2.34 g, 7.28 mmol) in accordance with Method P (alternative procedure) to give the title compound after column chromatography (silica gel, 80% EtOAc in heptanes) as a colorless viscous oil: LC/MS t_{R} 0.25 min; MS (ES+) m/z 124; ¹H NMR δ_{H} (360 MHz, CDCl₃) 8.54 (2H, d), 7.30 (2H, d), 4.91 (1H, q), 2.64 (1H, br s), 1.50 (3H, d).

### Example 105

### [5-(4-Methoxyphenyl)-6-methyl-7-(trifluoromethyl)pyrazolo[1,5-a]pyrimidin-3-yl][(2R)-2-methyl-4-[(1S)-1-(4-pyridinyl)ethyl]-1-piperazinyl]methanone (101)

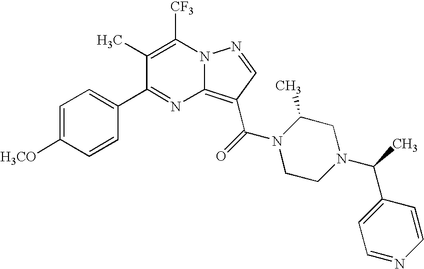

In accordance with Method S, alcohol 102 (43 mg, 0.35 mmol) was treated with MsCl (24 μL, 0.31 mmol, d 1.48) and TEA (87 μL, 0.62 mmol, d 0.726) to fashion the corresponding mesylate. Piperazine 69 (30 mg, 0.069 mmol) was treated immediately with this mesylate and TEA (48 μL, 0.35 mmol, d 0.726). Column chromatography (silica gel, 0-2% MeOH in DCM) afforded the title compound as a vitreous yellow solid: LC/MS t_{R} 1.58 min; MS (ES+) m/z 539, 561; ¹H NMR δ_{H} (250 MHz, CDCl₃) 8.53 (2H, br d), 8.41 (1H, s), 7.49 (2H, d), 7.27 (2H, dd), 7.03 (2H, d), 3.92 (3H, s), 3.61-5.46 (2H, br s), 3.37 (1H, br s), 3.33 (1H, m), 2.95 (1H, br s), 2.54 (3H, q), 2.39-2.58 (1H, br s), 2.07-2.29 (2H, m), 1.34 (3H, d), 1.28 (3H, d).

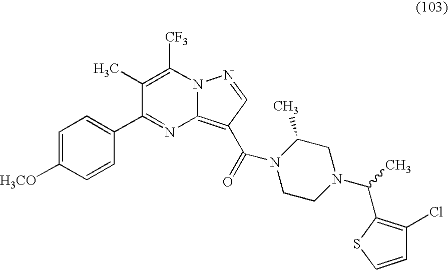

### Example 106

### α-Methyl-2-thiophenemethanol (104)

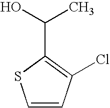

2-Acetyl-3-chlorothiophene (0.40 g, 2.49 mmol) was treated with (+)-DIP-Cl (1.76 g, 5.48 mmol) in accordance with Method P. Column chromatography (silica gel, 5-20% EtOAc in heptanes) followed by extraction of a methanolic solution (10 mL) of the purified compound with heptane (3×10 mL) and reduction of the MeOH in vacuo afforded the title compound admixed with α-pinene as a colorless oil: LC/MS t_{R} 1.57 min; MS (ES+) m/z 145, 147; ¹H NMR δ_{H} (250 MHz, CDCl₃) 7.21 (1H, d), 6.87 (1H, d), 5.28 (1H, q), 2.32 (1H, br s), 1.57 (3H, d).

### Example 107

### [(2R)-4-[1-(3-Chloro-2-thienyl)ethyl]-2-methyl-1-piperazinyl][5-(4-methoxyphenyl)-6-methyl-7-(trifluoromethyl)pyrazolo[1,5-a]pyrimidin-3-yl]methanone (103)

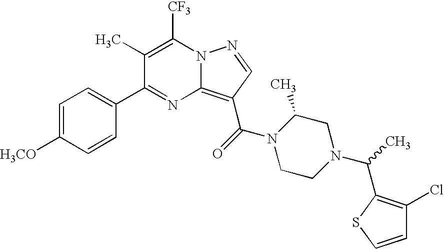

In accordance with Method S, alcohol 104 (88 mg, 0.35 mmol, ca. 64% purity) was treated with MsCl (24 μL, 0.31 mmol, d 1.48) and TEA (87 μL, 0.62 mmol, d 0.726) to fashion the corresponding mesylate. Piperazine 69 (30 mg, 0.069 mmol) was treated immediately with this mesylate and TEA (48 μL, 0.35 mmol, d 0.726). Column chromatography (silica gel, 20-50% EtOAc in heptanes) afforded the title compound (1:1 mixture of diastereomers) as a vitreous yellow solid: LC/MS t_{R} 2.09 and 2.13 min; MS (ES+) m/z 578, 580; ¹H NMR δ_{H} (250 MHz, CDCl₃) 8.42 and 8.41 (1H, 2 s), 7.50 (2H, d), 7.19 (1H, d), 7.03 (2H, d), 6.84 (1H, dd), 4.08-4.88 (1H, br s), 3.91 (3H, s), 3.78-4.03 (1H, obs m), 2.60-3.72 (3H, br m), 2.54 (3H, q), 2.16-2.45 (2H, m), 1.39 (3H, d), 1.33 (3H, d).

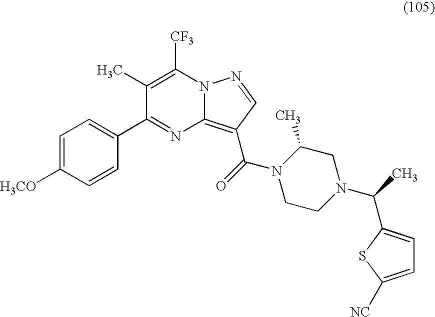

### Example 108

### 5-[(1R)-1-Hydroxyethyl]-2-thiophenecarbonitrile (106)

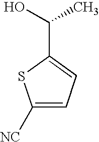

5-Acetyl-2-thiophenecarbonitrile (0.40 g, 2.65 mmol) was treated with (+)-DIP-Cl (1.87 g, 5.82 mmol) in accordance with Method P to afford the title compound after column chromatography (silica gel, 30-50% EtOAc in heptanes) as a yellow oil: LC/MS t_{R} 1.31 min; ¹H NMR δ_{H} (250 MHz, CDCl₃) 7.49 (1H, d), 6.94 (1H, d), 5.15 (1H, q), 2.44 (1H, br s), 1.60 (3H, d).

### Example 109

### 5-[(1S)-1-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]ethyl]-2-thiophenecarbonitrile (105)

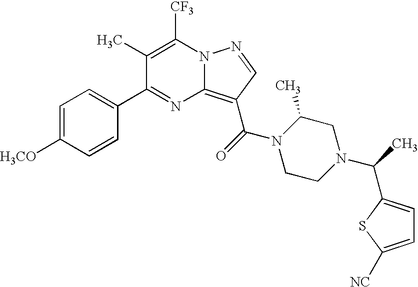

In accordance with Method S, alcohol 106 (66 mg, 0.35 mmol, ca. 80% purity) was treated with MsCl (24 μL, 0.31 mmol, d 1.48) and TEA (87 μL, 0.62 mmol, d 0.726) to fashion the corresponding mesylate. Piperazine 69 (30 mg, 0.069 mmol) was treated immediately with this mesylate and TEA (48 μL, 0.35 mmol, d 0.726). Column chromatography (silica gel, 60-90% EtOAc in heptanes) afforded the title compound as a vitreous yellow solid: LC/MS t_{R} 2.30 min; MS (ES+) m/z 569; ¹H NMR δ_{H} (250 MHz, CDCl₃) 8.44 (1H, s), 7.49 (2H, d), 7.47 (1H, d), 7.05 (2H, d), 6.85 (1H, d), 4.22-5.18 (1H, br s), 3.92 (3H, s), 3.79-4.22 (2H, m), 3.46 (1H, br s), 2.61-2.97 (3H, m), 2.54 (3H, q), 2.28-2.59 (1H, obs m), 1.34-1.52 (6H, m).

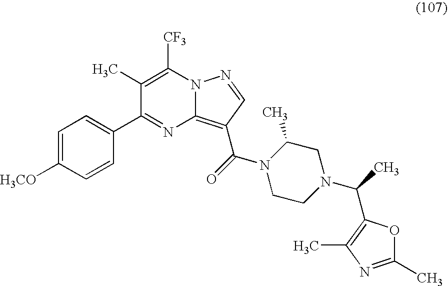

### Example 110

### (αR)-α,2,4-Trimethyl-5-oxazolemethanol (108)

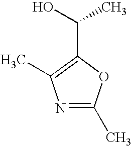

1-(2,4-Dimethyl-1,3-oxazol-5-yl)ethanone (0.40 g, 2.87 mmol) was treated with (+)-DIP-Cl (2.03 g, 5.32 mmol) in accordance with Method P to afford the title compound after column chromatography (silica gel, 40-100% EtOAc in heptanes) as a yellow oil: LC/MS t_{R} 0.82 min; MS (ES+) m/z 142; ¹H NMR δ_{H} (250 MHz, CDCl₃) 4.90 (1H, q), 2.38 (3H, s), 2.33 (1H, br s), 2.11 (3H, s), 1.53 (3H, d).

### Example 111

### [(2R)-4-[(1S)-1-(2,4-Dimethyl-5-oxazolyl)ethyl]-2-methyl-1-piperazinyl][5-(4-methoxyphenyl)-6-methyl-7-(trifluoromethyl)pyrazolo[1,5-a]pyrimidin-3-yl]methanone (107)

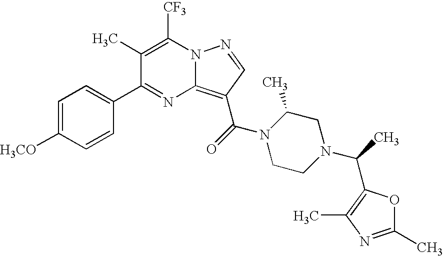

In accordance with Method S, alcohol 108 (51 mg, 0.36 mmol) was treated with MsCl (25 μL, 0.33 mmol, d 1.48) and TEA (90 μL, 0.65 mmol, d 0.726) to fashion the corresponding mesylate. Piperazine 69 (31 mg, 0.070 mmol) was treated immediately with this mesylate and TEA (50 μL, 0.36 mmol, d 0.726). Column chromatography (silica gel, 0-2% MeOH in DCM) afforded the title compound as a vitreous yellow solid: LC/MS t_{R} 1.59 min; MS (ES+) m/z 557, 579; ¹H NMR δ_{H} (360 MHz, CDCl₃) 8.41 (1H, s), 7.50 (2H, d), 7.03 (2H, d), 4.01-4.97 (2H, br s), 3.92 (3H, s), 3.64 (1H, m), 3.31 (1H, br s), 2.59-3.01 (2H, m), 2.54 (3H, q), 2.54 (1H, obs br s), 2.37 (3H, s), 2.22 (1H, m), 2.04 (3H, s), 1.39 (3H, d), 1.36 (3H, d).

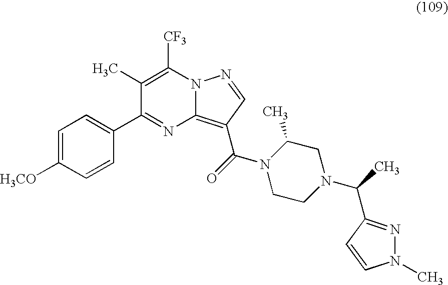

### Example 112

### (αR)-α,1-Dimethyl-1H-pyrazole-3-methanol (110)

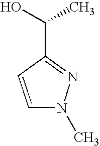

1-(1-Methyl-1H-pyrazol-3-yl)ethanone (0.40 g, 3.22 mmol) was treated with (+)-DIP-Cl (3.10 g, 9.67 mmol) in accordance with Method P to afford the title compound after column chromatography (silica gel, 30-100% EtOAc in heptanes, 5-10% MeOH in EtOAc) as a yellow oil: LC/MS t_{R} 0.67 min; MS (ES+) m/z 127; ¹H NMR δ_{H} (360 MHz, CDCl₃) 7.25 (1H, d), 6.16 (1H, d), 4.83 (1H, q), 3.80 (3H, s), 1.45 (3H, d).

### Example 113

### [5-(4-Methoxyphenyl)-6-methyl-7-(trifluoromethyl)pyrazolo[1,5-a]pyrimidin-3-yl][(2R)-2-methyl-4-[(1S)-1-(1-methyl-1H-pyrazol-3-yl)ethyl]-1-piperazinyl]methanone (109)

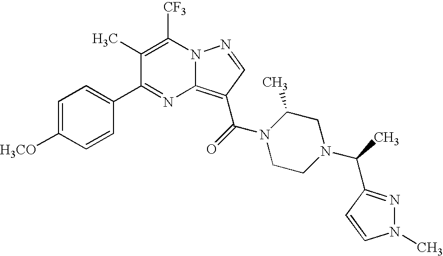

In accordance with Method S, alcohol 110 (44 mg, 0.35 mmol) was treated with MsCl (24 μL, 0.31 mmol, d 1.48) and TEA (87 μL, 0.62 mmol, d 0.726) to fashion the corresponding mesylate. Piperazine 69 (30 mg, 0.069 mmol) was treated immediately with this mesylate and TEA (48 μL, 0.35 mmol, d 0.726). Column chromatography (silica gel, 0-2% MeOH in DCM) afforded the title compound as a vitreous yellow solid: LC/MS t_{R} 1.54 min; MS (ES+) m/z 542; ¹H NMR δ_{H} (360 MHz, CD₃OD) 8.38 (1H, s), 7.62 (2H, d), 7.51 (1H, m), 7.14 (2H, d), 6.17 (1H, s), 4.09-4.79 (1H, br s), 3.95 (3H, s), 3.88 (1H, obs br s), 3.87 (3H, s), 3.70 (1H, m), 3.40 (1H, obs br s), 2.68-3.05 (2H, m), 2.57 (3H, q), 2.12-2.46 (2H, m), 1.40 (6H, 2 d).

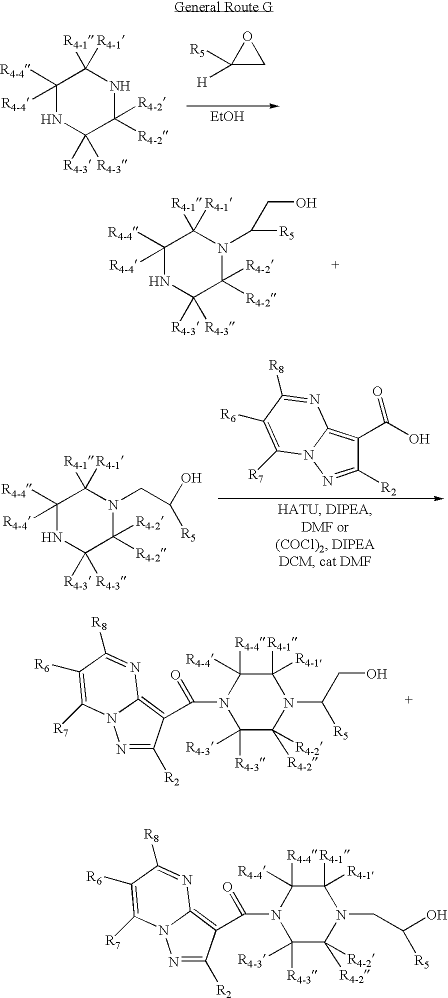

### Description of Methods Used in General Route G

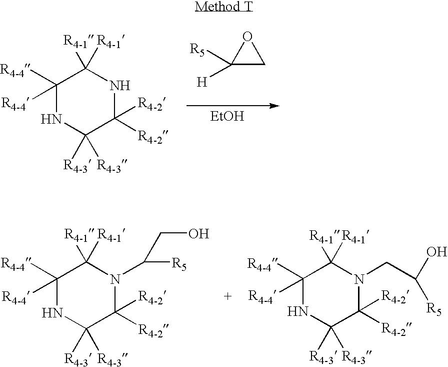

A stirred solution of the piperazine (1.2 equiv) in EtOH (20 vol) was warmed to 60° C. and treated with the epoxide (1 equiv) dropwise over 5-10 min. The reaction was heated to reflux for 2 h before assessing the reaction progress by LC/MS. On completion the solvent was evaporated and the residue purified by column chromatography (silica gel, 5-15% MeOH in EtOAc with 1% TEA) to afford various ratios of the desired alcohols.

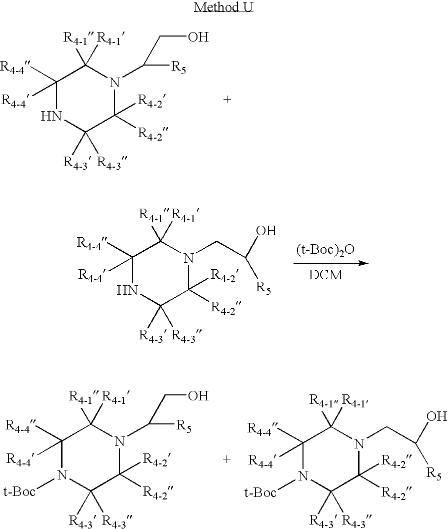

A solution of the mixture of piperazines formed via Method T (1 equiv) in DCM (10 vol) was treated with di-tert-butyl dicarbonate (1.1 equiv) and stirred at rt for 16 h. Reaction progress was monitored by LC/MS. On completion the reaction was diluted with satd NaHCO₃ (6 vol) and the organic phase isolated. The aqueous phase was extracted with DCM (2×6 vol). The organic phases were combined, dried (Na₂SO₄), filtered and reduced in vacuo. Column chromatography (silica gel, 0-50% EtOAc in heptanes) achieved the separation of the primary and secondary alcohols.

### Examples of Compounds Prepared via General Route G

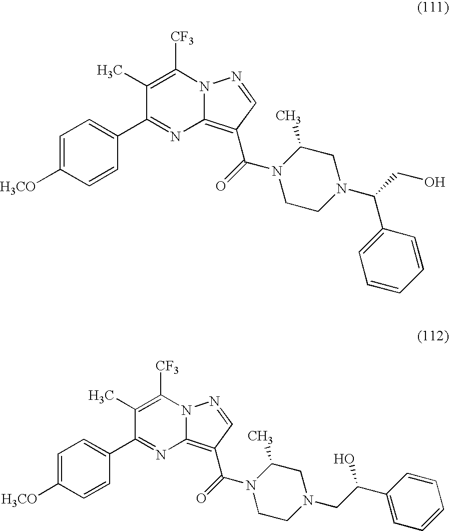

### Example 114

### (βS,3R)-3-Methyl-β-phenyl-1-piperazineethanol (113) and (αR,3R)-3-Methyl-α-phenyl-1-piperazineethanol (114)

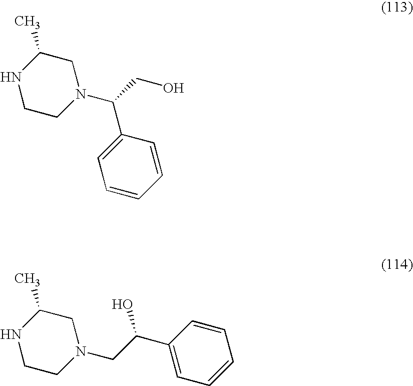

(R)-(+)-styrene oxide (200 mg, 1.67 mmol) was treated with (R)-(−)-2-methylpiperazine (200 mg, 2.00 mmol) in accordance with Method T to afford a 1:3 mixture of alcohols 113 and 114. Column chromatography (silica gel, 5-10% MeOH in EtOAc with 1% TEA) afforded 114 plus a 1:1 mixture of the title alcohols, both as a viscous caramel colored oils: for 113, LC/MS t_{R} 0.41 min; MS (ES+) m/z 221; ¹H NMR δ_{H} (360 MHz, CDCl₃) 7.14-7.41 (5H, m), 4.00 (1H, t), 3.68 (1H, d), 3.66 (1H, d), 2.95-3.00 (2H, m), 2.74-2.84 (2H, m), 2.40-2.62 (2H, br s), 2.25-2.39 (2H, m), 1.48 (1H, t), 0.97 (3H, d). For 114, LC/MS t_{R} 0.41 min; MS (ES+) m/z 221; ¹H NMR δ_{H} (360 MHz, CDCl₃) 7.22-7.41 (5H, m), 4.75 (1H, dd), 3.00-3.09 (2H, m), 2.86-2.99 (2H, m), 2.71 (1H, app dt), 2.52 (1H, dd), 2.43 (1H, dd), 2.23-2.68 (2H, br s), 2.10 (1H, app td), 2.01 (1H, t), 1.04 (3H, d).

### Example 115

### [(2R)-4-[(1S)-2-Hydroxy-1-phenylethyl]-2-methyl-1-piperazinyl][5-(4-methoxyphenyl)-6-methyl-7-(trifluoromethyl)pyrazolo[1,5-a]pyrimidin-3-yl]methanone (111) and [(2R)-4-[(2R)-2-Hydroxy-2-phenylethyl]-2-methyl-1-piperazinyl][5-(4-methoxyphenyl)-6-methyl-7-(trifluoromethyl)pyrazolo[1,5-a]pyrimidin-3-yl]methanone (112)

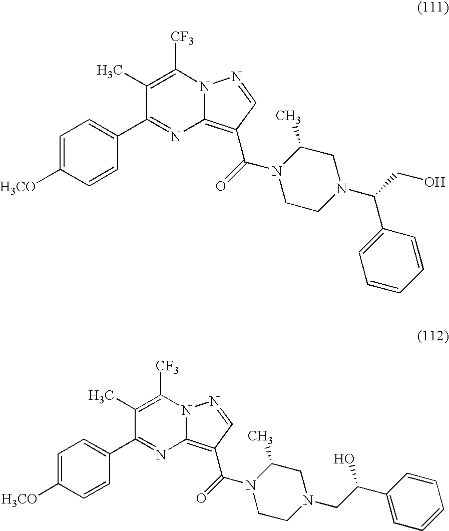

Carboxylic acid 19 (64 mg, 0.18 mmol) was treated with EDC (38 mg, 0.20 mmol), HOBT (27 mg, 0.20 mmol), and the 1:1 mixture of piperazines 113 and 114 (40 mg, 0.18 mmol) in accordance with Method G (alternative procedure). Purification by column chromatography (silica gel, 40-70% EtOAc in heptanes) separated the title compounds, both of which were vitreous yellow solids: for 111, LC/MS t_{R} 1.66 min; MS (ES+) m/z 554; ¹H NMR δ_{H} (360 MHz, CDCl₃) 8.40 (1H, s), 7.46 (2H, d), 7.32 (3H, m), 7.14 (2H, m), 7.03 (2H, d), 4.01 (1H, t), 3.93 (3H, s), 3.80-5.26 (2H, br m), 3.71 (1H, dd), 3.64 (1H, dd), 3.31 (1H, br s), 2.68-2.86 (2H, m), 2.64 (1H, dd), 2.53 (3H, q), 2.42 (1H, obs br s), 1.95 (1H, app td), 1.45 (3H, d). For 112, LC/MS t_{R} 1.65 min; MS (ES+) m/z 554; ¹H NMR δ_{H} (360 MHz, CDCl₃) 8.46 (1H, s), 7.52 (2H, d), 7.32-7.41 (5H, m), 7.03 (2H, d), 4.72 (1H, m), 3.97-5.23 (1H, br s), 3.90 (3H, s), 3.86 (1H, br s), 3.47 (1H, br s), 2.96 (1H, br s), 2.75 (1H, br s), 2.56 (3H, q), 2.44-2.58 (4H, m), 2.40 (1H, dd), 1.44 (3H, d).

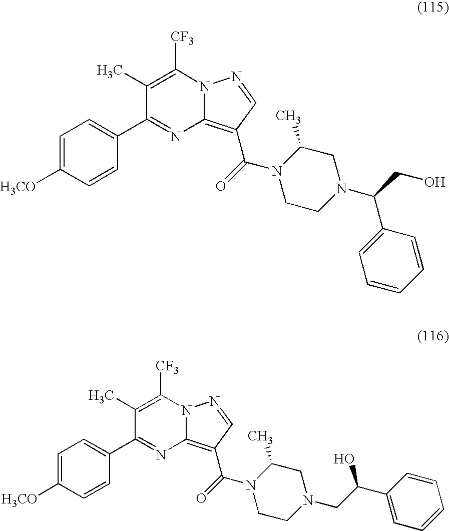

### Example 116

### (βR,3R)-3-Methyl-β-phenyl-1-piperazineethanol (117) and (αS,3R)-3-Methyl-α-phenyl-1-piperazineethanol (118)

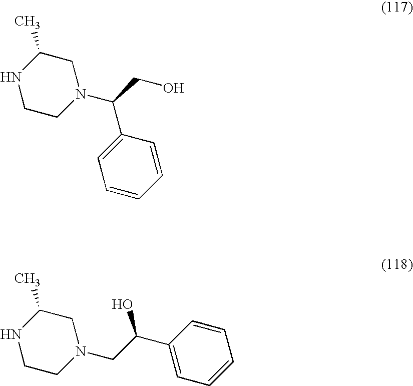

(S)-(−)-Styrene oxide (2.5 g, 20.8 mmol) was treated with (R)-(−)-2-methylpiperazine (2.5 g, 25 mmol) in accordance with Method T to afford a 1:3 mixture of alcohols 117 and 118. Column chromatography (silica gel, 5-15% MeOH in EtOAc with 1% TEA) afforded a 1:1 and 1:2 mixture of the title alcohols, both as a viscous caramel colored oils: LC/MS t_{R} 0.38 min; MS (ES+) m/z 221.

### Example 117

### [(2R)-4-[(1R)-2-Hydroxy-1-phenylethyl]-2-methyl-1-piperazinyl][5-(4-methoxyphenyl)-6-methyl-7-(trifluoromethyl)pyrazolo[1,5-a]pyrimidin-3-yl]methanone (115) and [(2R)-4-[(2S)-2-Hydroxy-2-phenylethyl]-2-methyl-1-piperazinyl][5-(4-methoxyphenyl)-6-methyl-7-(trifluoromethyl)pyrazolo[1,5-a]pyrimidin-3-yl]methanone (116)

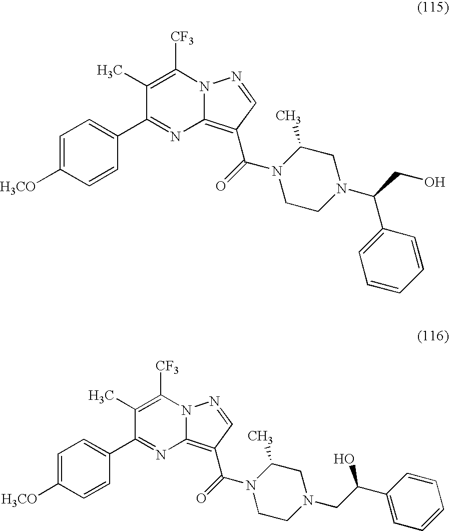

Carboxylic acid 19 (100 mg, 0.28 mmol) was treated with EDC (61 mg, 0.32 mmol), HOBT (43 mg, 0.32 mmol), and the 1:1 mixture of piperazines 117 and 118 (63 mg, 0.28 mmol) in accordance with Method G (alternative procedure). Purification by column chromatography (silica gel, 40-80% EtOAc in heptanes) separated the title compounds, both of which were isolated as vitreous yellow solids: for 115, LC/MS t_{R} 1.58 min; MS (ES+) m/z 554; ¹H NMR δ_{H} (250 MHz, CDCl₃) 8.39 (1H, s), 7.47 (2H, d), 7.31 (3H, m), 7.14 (2H, m), 7.03 (2H, d), 4.21-5.21 (1H, br s), 3.94 (1H, obs m), 3.93 (3H, s), 3.90 (1H, obs br s), 3.59-3.74 (2H, m), 3.46 (1H, br s), 2.90 (2H, br s), 2.64 (1H, br d), 2.53 (3H, q), 2.47 (1H, obs app td), 2.11 (1H, dd), 1.38 (3H, d). For 116, LC/MS t_{R} 1.62 min; MS (ES+) m/z 554; ¹H NMR δ_{H} (360 MHz, CDCl₃) 8.46 (1H, s), 7.52 (2H, d), 7.37 (4H, m), 7.30 (1H, m), 7.04 (2H, d), 4.77 (1H, m), 4.01-5.33 (1H, br s), 3.90 (3H, s), 3.88 (1H, obs br s), 3.41 (1H, br s), 3.09 (1H, br s), 2.63 (2H, br s), 2.56 (3H, q), 2.38-2.59 (3H, m), 2.24 (1H, app td), 1.43 (3H, d).

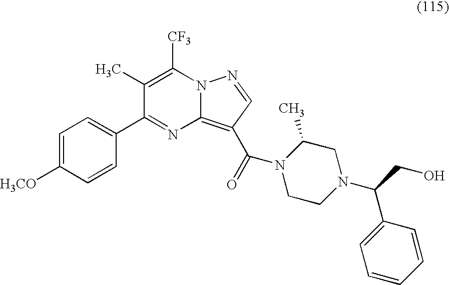

### Example 118

### (2R)-4-[(1R)-2-Hydroxy-1-phenylethyl]-2-methyl-1-piperazinecarboxylic Acid, 1,1-Dimethylethyl Ester (119) and (2R)-4-[(2S)-2-Hydroxy-2-phenylethyl]-2-methyl-1-piperazinecarboxylic Acid, 1,1-Dimethylethyl Ester (120)

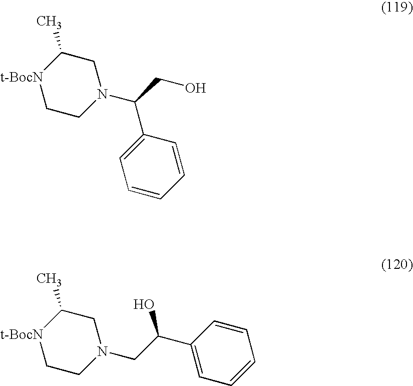

An approximate 3:5 mixture of piperazines 117 and 118 (24.3 g, 0.11 mol, prepared as per example 116 but without chromatographic separation) was treated with di-tert-butyl dicarbonate (26.5 g, 0.12 mol) in accordance with Method U. Column chromatography (silica gel, 0-50% EtOAc in heptanes) achieved the separation of 119 (low melting, white waxy solid) and 120 (white powder): for 119, LC/MS t_{R} 1.30 min; MS (ES+) m/z 265, 321; ¹H NMR δ_{H} (250 MHz, CDCl₃) 7.28-7.39 (3H, m), 7.13-7.22 (2H, m), 4.18 (1H, br s), 3.97 (1H, m), 3.85 (1H, br d), 3.63-3.74 (2H, m), 3.17 (1H, app td), 2.87 (1H, obs br s), 2.84 (1H, br d), 2.60 (1H, app dt), 2.30 (1H, app td), 1.95 (1H, dd), 1.41 (9H, s), 1.24 (3H, d). For 120, LC/MS t_{R} 1.25 min; MS (ES+) m/z 265, 321; ¹H NMR δ_{H} (250 MHz, CDCl₃) 7.11-7.33 (5H, m), 4.67 (1H, m), 4.15 (1H, br s), 3.79 (1H, d), 2.89-3.10 (2H, m), 2.26-2.50 (4H, m), 1.96 (1H, app td), 1.37 (9H, s), 1.19 (3H, d).

### Example 119

### (βR,3R)-3-Methyl-β-phenyl-1-piperazineethanol (117)

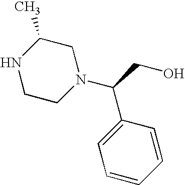

tert-Butyl carbamate 119 (5.24 g, 16.4 mmol) was treated with TFA in accordance with Method O to afford the title compound as a viscous caramel oil: LC/MS t_{R} 0.42 min; MS (ES+) m/z 221; ¹H NMR OH (360 MHz, CDCl₃) 7.26-7.37 (3H, m), 7.14-7.20 (2H, m), 3.98 (1H, m), 3.60-3.70 (2H, m), 2.87-2.97 (2H, m), 2.84 (1H, dd), 2.78 (1H, app dt), 2.70 (1H, app dq), 2.44 (2H, br s), 2.00 (1H, t), 1.81 (1H, app td), 1.02 (3H, d).

### Example 120

### [(2R)-4-[(1R)-2-Hydroxy-1-phenylethyl]-2-methyl-1-piperazinyl][5-(4-methoxyphenyl)-6-methyl-7-(trifluoromethyl)pyrazolo[1,5-a]pyrimidin-3-yl]methanone (115)

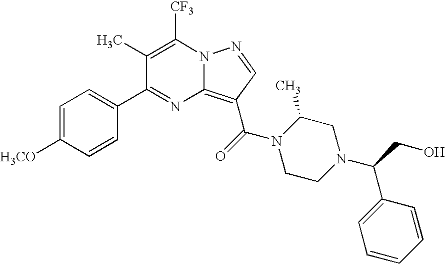

Carboxylic acid 19 (0.60 g, 1.72 mmol) was treated with HATU (0.79 g, 2.06 mmol), DIPEA (0.42 mL, 2.41 mmol, d 0.742) and piperazine 117 (0.40 g, 1.81 mmol) in accordance with Method G to afford the title compound after column chromatography (silica gel, 50-90% EtOAc in heptanes) as a vitreous yellow solid with spectral data consistent with that obtained for 115 as prepared in example 117.

### Example 121

### Alternate Procedure for the Preparation of [(2R)-4-[(1R)-2-Hydroxy-1-phenylethyl]-2-methyl-1-piperazinyl][5-(4-methoxyphenyl)-6-methyl-7-(trifluoromethyl)pyrazolo[1,5-a]pyrimidin-3-yl]methanone (115) Employing Method H

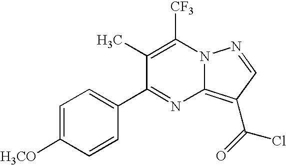

Oxalyl chloride (0.42 mL, 4.77 mmol, d 1.429) was added dropwise to an rt suspension of carboxylic acid 19 (1.12 g, 3.18 mmol) and DMF (3 drops) in DCM (16 mL). The reaction was stirred at rt for 2 h before reducing in vacuo providing 5-(4-methoxyphenyl)-6-methyl-7-(trifluoromethyl)pyrazolo[1,5-a]pyrimidine-3-carbonyl chloride as a yellow powder: ¹H NMR δ_{H} (250 MHz, CDCl₃) 8.68 (1H, s), 7.65 (2H, d), 7.06 (2H, d), 6.62 (1H, br s), 3.91 (3H, s), 2.64 (3H, q).

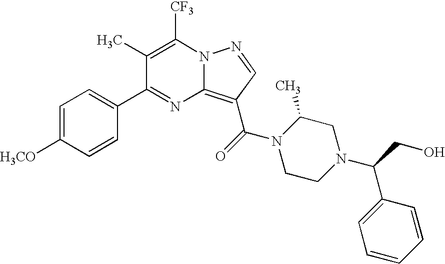

A stirred suspension of the acid chloride (1.18 g, 3.18 mmol) and DIPEA (1.38 mL, 7.95 mmol, d 0.742) in DCM (10 mL) was treated with a solution of piperazine 117 (0.77 g, 3.50 mmol) in DCM (6 mL) and the mixture stirred at rt for 1 h. Reaction progress was monitored by LC/MS. On completion the reaction mixture was diluted with satd NaHCO₃ (50 mL) and extracted with EtOAc (3×50 mL). The combined organic phases were washed with brine (25 mL), dried (MgSO₄) and filtered and the filtrate reduced in vacuo. Column chromatography (silica gel, 40-100% EtOAc in heptanes) afforded the title compound as a vitreous yellow solid with spectral data consistent with that obtained for 115 as prepared in examples 117 and 120.

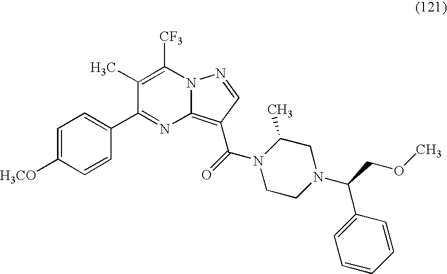

### Example 122

### (3R)-1-[(1R)-2-methoxy-1-phenylethyl]-3-methylpiperazine (122)

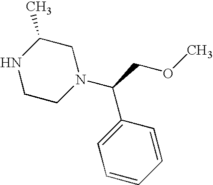

A suspension of sodium hydride (48 mg, 1.2 mmol, 60% dispersion in mineral oil) in THF (10 mL) at 0° C. was treated with a solution of piperazine 117 (220 mg, 1.0 mmol) in THF (3 mL) and stirred at rt for 2 h. A solution of iodomethane (62 mL, 1.0 mmol, d 2.28) in THF (2 mL) was added dropwise over 5 min and the reaction was stirred at rt a further 3 h. At this juncture, concd ammonia solution (1 mL) was added and the solvent removed in vacuo. Column chromatography (silica gel, 1-5% MeOH in DCM with 1% TEA) afforded the title compound as a viscous caramel colored oil: LC/MS t_{R} 0.26 min; MS (ES+) m/z 235; ¹H NMR δ_{H} (360 MHz, CDCl₃) 7.18-7.42 (5H, m), 4.42 (1H, dd), 3.24 (3H, s), 2.96-3.14 (4H, m), 2.90 (1H, d), 2.81 (1H, dd), 2.46 (1H, dd), 2.32 (1H, m), 2.25 (1H, br s), 1.98 (1H, t), 1.16 (3H, d).

### Example 123

### [(2R)-4-[(1R)-2-Methoxy-1-phenylethyl]-2-methyl-1-piperazinyl][5-(4-methoxyphenyl)-6-methyl-7-(trifluoromethyl)pyrazolo[1,5-a]pyrimidin-3-yl]methanone (121)

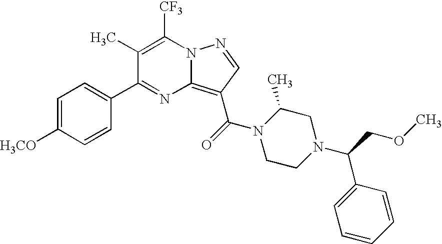

Carboxylic acid 19 (27 mg, 0.077 mmol) was treated with EDC (15 mg, 0.077 mmol), HOBT (11 mg, 0.077 mmol) and piperazine 122 (15 mg, 0.064 mmol) in accordance with Method G (alternative procedure) to afford the title compound after column chromatography (silica gel, 30-40% EtOAc in heptanes) as a vitreous yellow solid: LC/MS t_{R} 1.74 min; MS (ES+) m/z 568; ¹H NMR δ_{H} (360 MHz, CDCl₃) 8.41 (1H, s), 7.51 (2H, d), 7.22-7.37 (5H, m), 7.03 (2H, d), 3.93 (3H, s), 3.85-5.15 (2H, br s), 3.74 (1H, dd), 3.58 (1H, dd), 3.49 (1H, dd), 3.44 (1H, obs br s), 3.29 (3H, s), 3.08 (1H, br s), 2.54 (3H, q), 2.50 (1H, br s), 2.34 (1H, app td), 2.25 (1H, dd), 1.33 (3H, d).

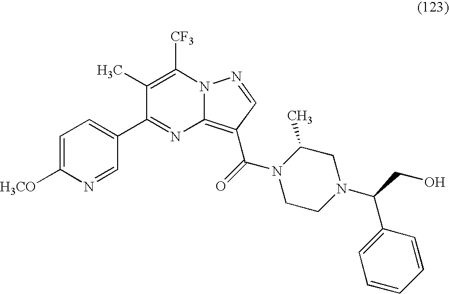

### Example 124

### 1-(6-Methoxy-3-pyridinyl)-1-propanone (124)

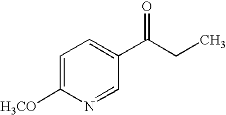

A solution of 5-bromo-2-methoxypyridine (3.88 mL, 30 mmol, d 1.453) in Et₂O (35 mL) was cooled to −78° C. and treated with n-BuLi (18.9 mL, 30.3 mmol, 1.6 M solution in hexanes) over 5-10 min, maintaining the internal temperature below −65° C. during the addition. After 15 min stirring at −78° C., propionitrile (2.30 mL, 32.1 mmol, d 0.77) was added and the reaction was removed from the cooling bath and allowed to warm to rt over 1 h. Aqueous NH₄Cl (30 mL) was added followed 1-2 min later with EtOAc (30 mL) and the two phases were separated. The aqueous phase was extracted with EtOAc (2×30 mL), the organic phases combined, dried (MgSO₄), filtered and reduced in vacuo. Column chromatography (silica gel, 5-20% EtOAc in heptanes) provided the title compound as a white waxy solid: LC/MS t_{R} 1.59 min; MS (ES+) m/z 166, 207; ¹H NMR δ_{H} (250 MHz, CDCl₃) 8.79 (1H, d), 8.14 (1H, dd), 6.78 (1H, d), 4.00 (3H, s), 2.95 (2H, q), 1.48 (3H, t).

### Example 125

### 4,4,4-Trifluoro-1-(6-methoxy-3-pyridinyl)-2-methyl-1,3-butanedione (125)

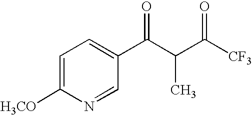

Ketone 124 (1.0 g, 6.05 mmol) was treated with LHMDS (12.1 mL, 12.1 mmol, 1 M solution in THF) and 1-(trifluoroacetyl)imidazole (2.76 mL, 24.2 mmol, d 1.441) using Method C to afford the title compound after column chromatography (silica gel, 5-20% EtOAc in heptanes) as a pale yellow viscous oil; by NMR, the title compound exists as a ˜3:7 mixture with its hydrate: LC/MS t_{R} 1.62 min; MS (ES+) m/z 262, 280; ¹H NMR δ_{H} (250 MHz, CDCl₃) 8.84 and 8.80 (1H, 2 d), 8.15 and 8.14 (1H, 2 dd), 6.86 and 6.84 (1H, 2 d), 5.40-6.60 (0.7H, br m), 4.82 and 3.96 (1H, 2 q), 4.04 (3H, s), 1.55 and 1.38 (3H, 2 d).

### Example 126

### 5-(6-Methoxy-3-pyridinyl)-6-methyl-7-(trifluoromethyl)pyrazolo[1,5-a]pyrimidine-3-carboxylic Acid, 3-Propenyl Ester (126)

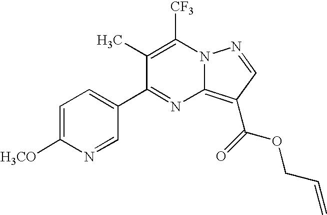

Aminopyrazole 17 (1.47 g, 8.76 mmol) was treated with diketone 125 (1.37 g, 5.26 mmol) using Method D to give the title compound after column chromatography (silica gel, 10-40% EtOAc in heptanes) an orange waxy solid: LC/MS t_{R} 2.19 min; MS (ES+) m/z 393, 434, 807; ¹H NMR δ_{H} (250 MHz, CDCl₃) 8.62 (1H, s), 8.47 (1H, d), 7.92 (1H, dd), 6.89 (1H, d), 5.93-6.13 (1H, m), 5.47 (1H, app dq), 5.27 (1H, app dq), 4.85 (2H, app dt), 4.02 (3H, s), 2.60 (3H, q).

### Example 127

### 5-(6-Methoxy-3-pyridinyl)-6-methyl-7-(trifluoromethyl)pyrazolo[1,5-a]pyrimidine-3-carboxylic Acid (127)

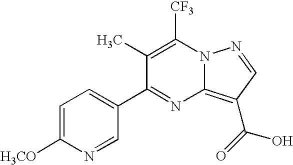

Pyrazolopyrimidine allyl ester 126 (1.11 g, 2.83 mmol) was treated with 1,3-dimethylbarbituric acid (0.44 g, 2.83 mmol) and Pd(PPh₃)₄ (0.33 g, 0.28 mmol) using Method E. On completion the reaction was filtered and the filter cake washed with THF (5 mL) to afford on drying the title compound as a white solid: LC/MS t_{R} 1.81 min; MS (ES+) m/z 353, 394; ¹H NMR δ_{H} (250 MHz, DMSO-d₆) 8.66 (1H, s), 8.46 (1H, d), 8.01 (1H, dd), 7.01 (1H, d), 3.95 (3H, s), 2.47 (3H, obs q).

### Example 128

### [(2R)-4-[(1R)-2-Hydroxy-1-phenylethyl]-2-methyl-1-piperazinyl][5-(6-methoxy-3-pyridinyl)-6-methyl-7-(trifluoromethyl)pyrazolo[1,5-a]pyrimidin-3-yl]methanone (123)

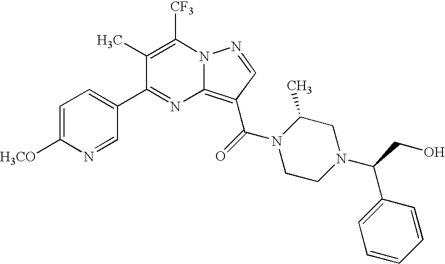

Carboxylic acid 127 (100 mg, 0.28 mmol) was treated with HATU (130 mg, 0.34 mmol), DIPEA (69 μL, 0.40 mmol, d 0.742) and piperazine 117 (66 mg, 0.30 mmol) in accordance with Method G to afford the title compound after column chromatography (silica gel, 60-80% EtOAc in heptanes) as a vitreous yellow solid: LC/MS t_{R} 1.49 min; MS (ES+) m/z 555; ¹H NMR δ_{H} (250 MHz, CDCl₃) 8.41 (1H, s), 8.37 (1H, d), 7.75 (1H, dd), 7.27-7.39 (3H, m), 7.09-7.20 (2H, m), 6.88 (1H, d), 4.11-5.36 (1H, br s), 4.08 (3H, s), 3.98 (1H, t), 3.72 (1H, d), 3.69 (1H, d), 3.48 (1H, br s), 2.91 (1H, br d), 2.55 (3H, q), 2.20-2.74 (3H, m), 2.10 (1H, dd), 1.39 (3H, d).

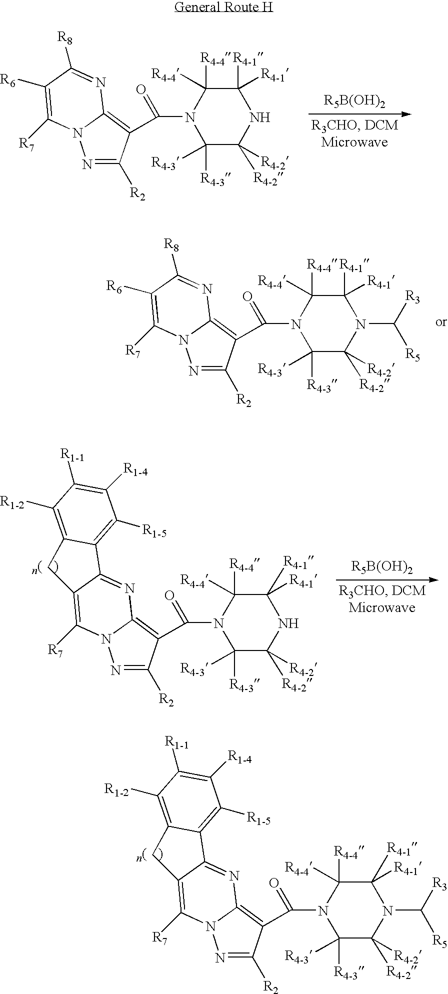

### Description of Method Used in General Route H

### Method V

A solution of the piperazine (1 equiv) in DCM (50 vol) was treated with the appropriate aldehyde or aldehyde dimer (0.50-1.1 equiv) followed by the appropriate boronic acid (1.1 equiv) and heated to 70° C. for 20-30 min using a microwave reactor. Reaction progress was assessed by LC/MS. If the reaction was incomplete, further aliquots of aldehyde or aldehyde dimer (0.50-1.1 equiv) and boronic acid (1.1 equiv) were added and heating was recommenced for 20-30 min, whereupon reaction progress was again assessed by LC/MS. Where necessary, this process was repeated until sufficient conversion to the product had been achieved. The reaction mixture was allowed to cool to rt and evaporated to dryness. Column chromatography (EtOAc in heptanes or MeOH in DCM) or reverse phase preparative HPLC (5-95% MeCN in water) of the residue afforded the desired product.

### Examples of Compounds Prepared Via General Route H

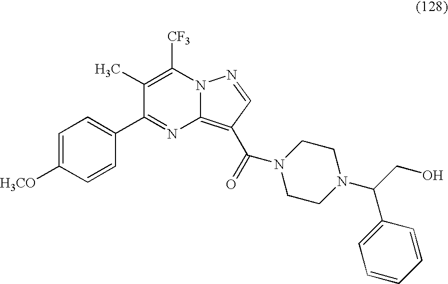

### Example 129

### 4-[[5-(4-Methoxyphenyl)-6-methyl-7-(trifluoromethyl)pyrazolo[1,5-a]pyrimidin-3-yl]carbonyl]-1-piperazinecarboxylic Acid, 1,1-Dimethylethyl Ester (129)

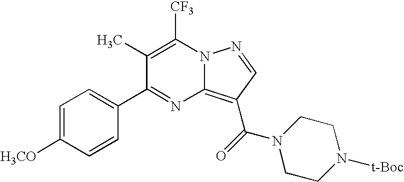

Carboxylic acid 19 (150 mg, 0.43 mmol) was treated with HATU (195 mg, 0.51 mmol), DIPEA (100 μL, 0.60 mmol, d 0.742) and 1-t-Boc piperazine (88 mg, 0.47 mmol) in accordance with Method G to afford the title compound as a tan powder: LC/MS t_{R} 2.31 min; MS (ES+) m/z 520, 542; ¹H NMR δ_{H} (360 MHz, CDCl₃) 8.48 (1H, s), 7.50 (2H, d), 7.05 (2H, d), 3.91 (3H, s), 3.75 (2H, br s), 3.66 (2H, br s), 3.50 (4H, br s), 2.55 (3H, q), 1.48 (9H, s).

### Example 130

### [5-(4-Methoxyphenyl)-6-methyl-7-(trifluoromethyl)pyrazolo[1,5-a]pyrimidin-3-yl][1-piperazinyl]methanone (130)

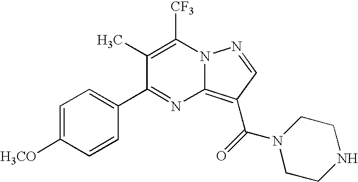

tert-Butyl carbamate 129 (190 mg, 0.37 mmol) was treated with TFA in accordance with Method O to afford the title compound as a glassy orange solid: LC/MS t_{R} 1.36 min; MS (ES+) m/z 420; ¹H NMR δ_{H} (250 MHz, CDCl₃) 8.46 (1H, s), 7.53 (2H, d), 7.04 (2H, d), 3.90 (3H, s), 3.77 (2H, br s), 3.65 (2H, br s), 2.93 (4H, br s), 2.55 (3H, q).

### Example 131

### [4-(2-Hydroxy-1-phenylethyl)-1-piperazinyl][5-(4-methoxyphenyl)-6-methyl-7-(trifluoromethyl)pyrazolo[1,5-a]pyrimidin-3-yl]methanone (128)

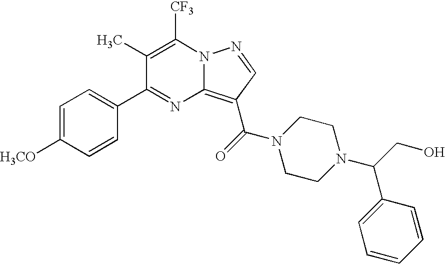

Piperazine 130 (50 mg, 0.12 mmol), glycolaldehyde dimer (8 mg, 0.06 mmol) and phenylboronic acid (16 mg, 0.13 mmol) were reacted in accordance with Method V and purified by column chromatography (silica gel, 0-2% MeOH in DCM) to afford the title compound as a vitreous yellow solid: LC/MS t_{R} 1.52 min; MS (ES+) m/z 540; ¹H NMR δ_{H} (360 MHz, CDCl₃) 8.41 (1H, s), 7.48 (2H, d), 7.31 (3H, m), 7.14 (2H, m), 7.05 (2H, d), 4.00 (1H, m), 3.93 (3H, s), 3.82 (2H, br s), 3.70 (4H, m), 2.95 (1H, br s), 2.64 (2H, br s), 2.53 (3H, q), 2.43 (2H, br s).

### Example 132

### [(2R)-4-[(1S)-1-(2-Furanyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (131) and [(2R)-4-[(1R)-1-(2-Furanyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (132)

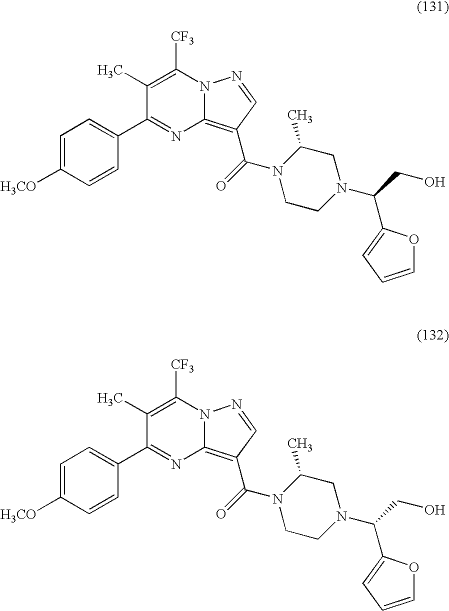

Piperazine 69 (100 mg, 0.23 mmol), glycolaldehyde dimer (15 mg, 0.13 mmol) and 2-furanylboronic acid (28 mg, 0.25 mmol) were reacted in accordance with Method V to give an ˜1:1 mixture of amides 131 and 132 which were separated by column chromatography (silica gel, 0-1% MeOH in DCM) and isolated as vitreous yellow solids: for 131, 57% de; LC/MS t_{R} 1.58 min; MS (ES+) m/z 544; ¹H NMR δ_{H} (360 MHz, CDCl₃) 8.42 (1H, s), 7.48 (2H, d), 7.33 (1H, s), 7.03 (2H, s), 6.32 (1H, m), 6.10 (1H, m), 4.54 (1H, br s), 3.92 (3H, s), 3.77-3.92 (2H, m), 3.68 (1H, m), 3.44 (1H, br s), 3.13 (1H, br s), 2.58-2.97 (3H, m), 2.54 (3H, q), 2.10 (1H, dd), 1.38 (3H, d). For 132, >95% de; LC/MS t_{R} 1.61 min; MS (ES+) m/z 544; ¹H NMR δ_{H} (360 MHz, CDCl₃) 8.42 (1H, s), 7.48 (2H, d), 7.33 (1H, s), 7.03 (2H, s), 6.32 (1H, m), 6.10 (1H, m), 4.50 (1H, br s), 3.92 (3H, s), 3.91 (1H, m), 3.81 (1H, m), 3.71 (1H, m), 3.09-3.46 (2H, br m), 2.91 (1H, m), 2.60-2.83 (2H, m), 2.54 (3H, q), 1.95 (1H, app td), 1.42 (3H, d).

### Example 133

### [(2R)-4-[(1S)-2-Hydroxy-1-(2-thienyl)ethyl]-2-methyl-1-piperazinyl][5-(4-methoxyphenyl)-6-methyl-7-(trifluoromethyl)pyrazolo[1,5-a]pyrimidin-3-yl]methanone (133) and [(2R)-4-[(1R)-2-Hydroxy-1-(2-thienyl)ethyl]-2-methyl-1-piperazinyl][5-(4-methoxyphenyl)-6-methyl-7-(trifluoromethyl)pyrazolo[1,5-a]pyrimidin-3-yl]methanone (134)

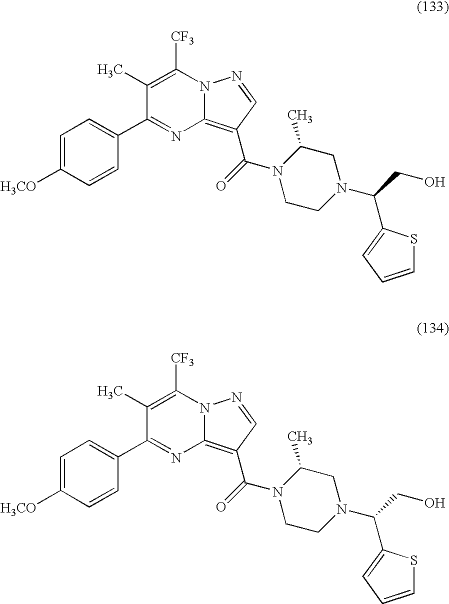

Piperazine 69 (50 mg, 0.12 mmol), glycolaldehyde dimer (8 mg, 0.06 mmol) and 2-thienylboronic acid (16 mg, 0.13 mmol) were reacted in accordance with Method V and purified by column chromatography (silica gel, 50% EtOAc in heptanes) to afford a 1:1 mixture of the title compounds as a vitreous yellow solid: LC/MS t_{R} 1.62 and 1.66 min; MS (ES+) m/z 560; ¹H NMR δ_{H} (250 MHz, CDCl₃) 8.42 and 8.41 (1H, 2 s), 7.47 (2H, d), 7.27 (1H, m), 7.03 (2H, d), 7.01 (1H, m), 6.83 (1H, m), 4.12-4.84 (1H, br m), 3.93 (3H, s), 3.80-4.11 (3H, m), 3.74 (1H, m), 3.07-3.60 (1H, br m), 2.54 (3H, q), 2.40-2.87 (3H, m), 2.21 (0.5H, dd), 2.06 (0.5H, app td), 1.43 and 1.39 (3H, 2 d).

### Example 134

### 1-[5-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]-2-thienyl]ethanone (135) and 1-[5-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]-2-thienyl]ethanone (136)

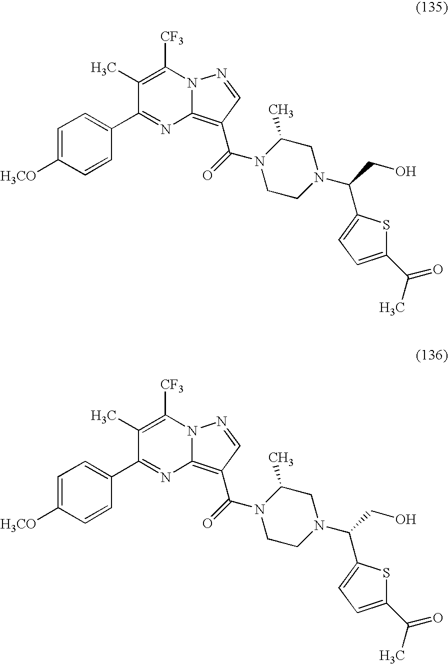

Piperazine 69 (50 mg, 0.12 mmol), glycolaldehyde dimer (8 mg, 0.06 mmol) and 5-acetyl-2-thienylboronic acid (20 mg, 0.13 mmol) were reacted in accordance with Method V and purified by column chromatography (silica gel, 50-70% EtOAc in heptanes) to give partial separation of the title diastereomers as vitreous yellow solids: for 135, 32% de; LC/MS t_{R} 1.85 min; MS (ES+) m/z 602, 624; ¹H NMR δ_{H} (360 MHz, CDCl₃) 8.43 (1H, s), 7.59 (1H, d), 7.48 (2H, d), 7.06 (2H, d), 6.86 (1H, d), 4.16-4.89 (1H, br m), 4.01 (1H, m), 3.95 (3H, s), 3.89 (1H, m), 3.75 (1H, m), 3.43 (1H, br s), 2.58-2.98 (4H, m), 2.55 (3H, s), 2.53 (3H, q), 2.27 (1H, dd), 1.40 (3H, d). For 136, 48% de; LC/MS t_{R} 1.89 min; MS (ES+) m/z 602, 624; ¹H NMR δ_{H} (360 MHz, CDCl₃) 8.43 (1H, s), 7.59 (1H, d), 7.48 (2H, d), 7.06 (2H, d), 6.86 (1H, d), 4.20-4.90 (1H, br m), 3.95 (3H, s), 3.93 (2H, m), 3.77 (1H, m), 3.37 (1H, br m), 2.58-3.02 (3H, m), 2.55 (3H, s), 2.54 (3H, q), 2.54 (1H, obs m), 2.10 (1H, app td), 1.43 (3H, d).

### Example 135

### [(2R)-4-[(1S)-2-Hydroxy-1-(4-methyl-2-thienyl)ethyl]-2-methyl-1-piperazinyl][5-(4-methoxyphenyl)-6-methyl-7-(trifluoromethyl)pyrazolo[1,5-a]pyrimidin-3-yl]methanone (137) and [(2R)-4-[(1R)-2-Hydroxy-1-(4-methyl-2-thienyl)ethyl]-2-methyl-1-piperazinyl][5-(4-methoxyphenyl)-6-methyl-7-(trifluoromethyl)pyrazolo[1,5-a]pyrimidin-3-yl]methanone (138)

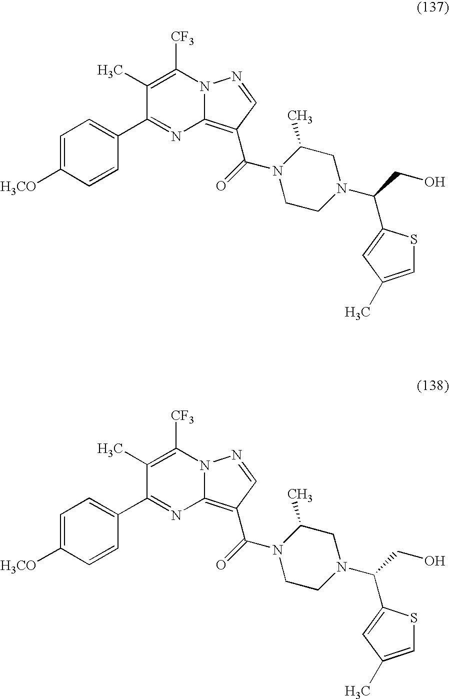

Piperazine 69 (50 mg, 0.12 mmol), glycolaldehyde dimer (8 mg, 0.06 mmol) and 4-methyl-2-thienylboronic acid (18 mg, 0.13 mmol) were reacted in accordance with Method V to afford the title compounds after reverse phase preparative HPLC (5-95% MeCN in water) as vitreous yellow solids: for 137, >80% de; LC/MS t_{R} 1.68 min; MS (ES+) m/z 574; ¹H NMR δ_{H} (360 MHz, CDCl₃) 8.41 (1H, s), 7.48 (2H, d), 7.03 (2H, d), 6.82 (1H, s), 6.64 (1H, s), 4.66 (1H, br m), 3.97 (1H, m), 3.92 (3H, s), 3.83 (1H, m), 3.70 (1H, br s), 3.45 (1H, br s), 3.09 (1H, br s), 2.76 (1H, br s), 2.57-2.71 (2H, m), 2.54 (3H, q), 2.23 (3H, s), 2.23 (1H, obs dd), 1.39 (3H, d). For 138, >82% de; LC/MS t_{R} 1.75 min; MS (ES+) m/z 574; ¹H NMR δ_{H} (360 MHz, CDCl₃) 8.41 (1H, s), 7.49 (2H, d), 7.03 (2H, d), 6.82 (1H, s), 6.64 (1H, s), 4.06-4.92 (1H, br s), 3.92 (3H, s), 3.79-4.04 (3H, obs m), 3.72 (1H, m), 3.33 (1H, br s), 3.20 (1H, br s), 2.83 (1H, dd), 2.74 (1H, m), 2.64 (1H, br d), 2.54 (3H, q), 2.23 (3H, s), 2.08 (1H, app td), 1.43 (3H, d).

### Example 136

### [(2R)-4-[(1S)-1-(5-Chloro-2-thienyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (139) and [(2R)-4-[(1R)-1-(5-Chloro-2-thienyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (140)

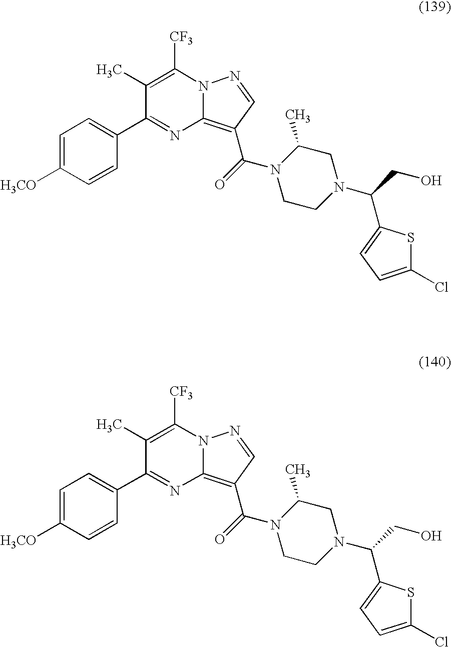

Piperazine 69 (50 mg, 0.12 mmol), glycolaldehyde dimer (8 mg, 0.06 mmol) and 5-chloro-2-thienylboronic acid (21 mg, 0.13 mmol) were reacted in accordance with Method V to afford the title compounds after reverse phase preparative HPLC (5-95% MeCN in water) as vitreous yellow solids: for 139, >95% de; LC/MS t_{R} 1.93 min; MS (ES+) m/z 594; ¹H NMR δ_{H} (250 MHz, CDCl₃) 8.44 (1H, s), 7.48 (2H, d), 7.05 (2H, d), 6.81 (1H, d), 6.61 (1H, d), 4.66 (1H, br s), 4.20 (1H, br s), 3.93 (3H, s), 3.65-3.96 (3H, m), 3.44 (1H, br s), 2.55-3.04 (4H, m), 2.54 (3H, q), 2.31 (1H, dd), 1.39 (3H, d). For 140, >85% de; LC/MS t_{R} 1.99 min; MS (ES+) m/z 594; ¹H NMR δ_{H} (250 MHz, CDCl₃) 8.44 (1H, s), 7.48 (2H, d), 7.05 (2H, d), 6.81 (1H, d), 6.61 (1H, d), 3.99-5.09 (1H, br m), 3.93 (3H, s), 3.57-3.98 (4H, m), 3.32 (1H, br s), 2.58-2.85 (3H, m), 2.54 (3H, q), 2.29-2.58 (1H, br m), 2.14 (1H, dd), 1.41 (3H, d).

### Example 137

### [(2R)-4-[(1R)-2-Hydroxy-1-(3-thienyl)ethyl]-2-methyl-1-piperazinyl][5-(4-methoxyphenyl)-6-methyl-7-(trifluoromethyl)pyrazolo[1,5-a]pyrimidin-3-yl]methanone (141) and [(2R)-4-[(1S)-2-Hydroxy-1-(3-thienyl)ethyl]-2-methyl-1-piperazinyl][5-(4-methoxyphenyl)-6-methyl-7-(trifluoromethyl)pyrazolo[1,5-a]pyrimidin-3-yl]methanone (142)

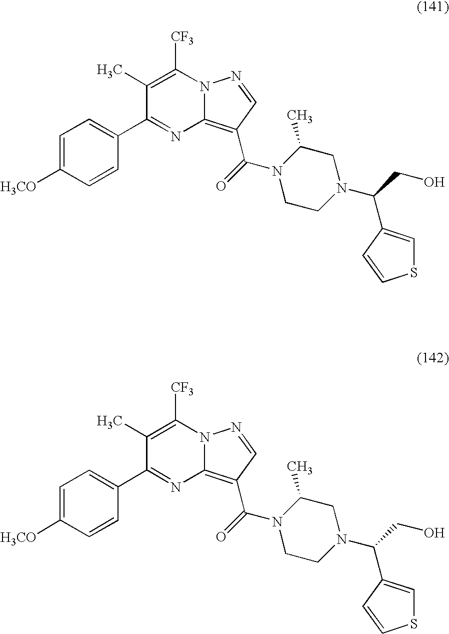

Piperazine 69 (50 mg, 0.12 mmol), glycolaldehyde dimer (8 mg, 0.06 mmol) and 3-thienylboronic acid (16 mg, 0.13 mmol) were reacted in accordance with Method V and purified by column chromatography (silica gel, 0-2% MeOH in DCM) to afford a 1:1 mixture of the title compounds as a vitreous yellow solid: LC/MS t_{R} 1.54 min; MS (ES+) m/z 560; ¹H NMR δ_{H} (250 MHz, CDCl₃) 8.41 and 8.40 (1H, 2 s), 7.46 (2H, d), 7.29 (1H, m), 7.04 (2H, d), 7.01 (1H, obs m), 6.88 (1H, d), 4.01-4.97 (1H, br m), 3.93 (3H, s), 3.56-4.01 (4H, m), 3.40 (1H, br s), 2.45-2.89 (3H, m), 2.53 (3H, q), 2.11 (0.5H, dd), 1.96 (0.5H, dd), 1.42 and 1.39 (3H, 2 d).

### Example 138

### [(2R)-4-[(1R)-2-Hydroxy-1-(2-methoxy-3-pyridinyl)ethyl]-2-methyl-1-piperazinyl][5-(4-methoxyphenyl)-6-methyl-7-(trifluoromethyl)pyrazolo[1,5-a]pyrimidin-3-yl]methanone (143) and [(2R)-4-[(1S)-2-Hydroxy-1-(2-methoxy-3-pyridinyl)ethyl]-2-methyl-1-piperazinyl][5-(4-methoxyphenyl)-6-methyl-7-(trifluoromethyl)pyrazolo[1,5-a]pyrimidin-3-yl]methanone (144)

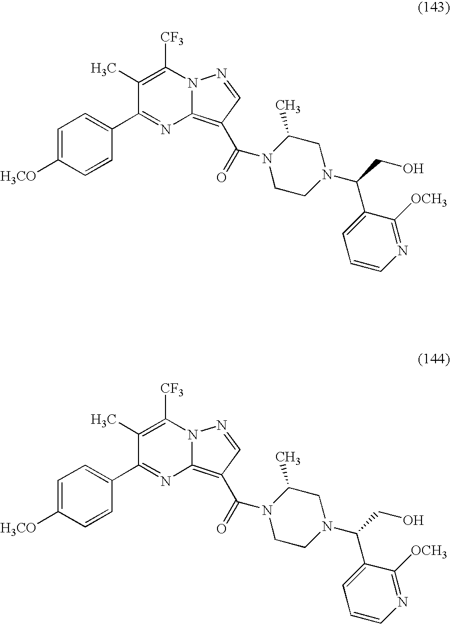

Piperazine 69 (50 mg, 0.12 mmol), glycolaldehyde dimer (8 mg, 0.06 mmol) and 2-methoxy-3-pyridinylboronic acid (20 mg, 0.13 mmol) were reacted in accordance with Method V and purified by column chromatography (silica gel, 0-2% MeOH in DCM) to provide the title diastereomers as vitreous yellow solids: for 143, 40% de; LC/MS t_{R} 1.56 min; MS (ES+) m/z 585; ¹H NMR δ_{H} (250 MHz, CDCl₃) 8.40 (1H, s), 8.12 (1H, m), 7.47 (2H, d), 7.41 (1H, obs m), 7.02 (2H, d), 6.85 (1H, m), 4.64 (1H, br m), 4.17 (1H, m), 4.01 (1H, m), 3.93 (3H, s), 3.90 (3H, s), 3.88 (1H, obs m), 3.63 (1H, m), 3.46 (1H, br s), 2.59-3.14 (3H, m), 2.54 (3H, q), 2.43 (1H, m), 2.14 (1H, dd), 1.39 (3H, d). For 144, 41% de; LC/MS t_{R} 1.60 min; MS (ES+) m/z 585; ¹H NMR δ_{H} (360 MHz, CDCl₃) 8.39 (1H, s), 8.12 (1H, dd), 7.47 (2H, d), 7.41 (1H, m), 7.02 (2H, d), 6.85 (1H, m), 4.31 (1H, br m), 4.15 (1H, m), 4.01 (1H, m), 3.94 (3H, s), 3.91 (1H, obs m), 3.90 (3H, s), 3.63 (1H, m), 3.14-3.54 (1H, br m), 2.71-3.04 (2H, m), 2.60 (1H, dd), 2.54 (3H, q), 2.40 (1H, m), 1.98 (1H, app td), 1.44 (3H, d).

### Example 139

### [(2R)-4-[(1R)-2-Hydroxy-1-(2-methoxyphenyl)ethyl]-2-methyl-1-piperazinyl][5-(4-methoxyphenyl)-6-methyl-7-(trifluoromethyl)pyrazolo[1,5-a]pyrimidin-3-yl]methanone (145) and [(2R)-4-[(1S)-2-Hydroxy-1-(2-methoxyphenyl)ethyl]-2-methyl-1-piperazinyl][5-(4-methoxyphenyl)-6-methyl-7-(trifluoromethyl)pyrazolo[1,5-a]pyrimidin-3-yl]methanone (146)

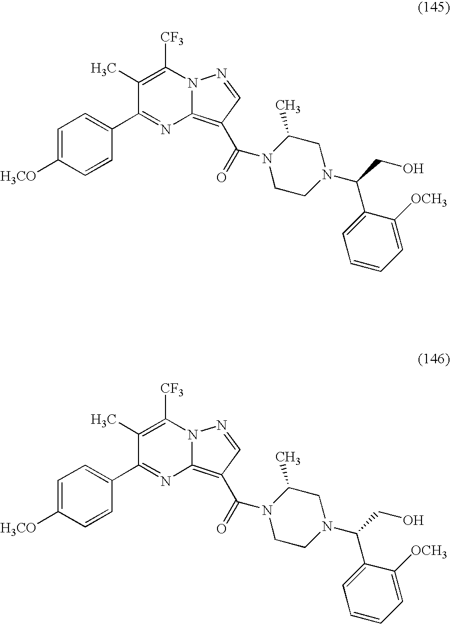

Piperazine 69 (100 mg, 0.23 mmol), glycolaldehyde dimer (15 mg, 0.13 mmol) and 2-methoxyphenylboronic acid (19 mg, 0.13 mmol) were reacted in accordance with Method V and purified by column chromatography (silica gel, 50-70% EtOAc in heptanes) to provide the title diastereomers as vitreous yellow solids: for 145, 40% de; LC/MS t_{R} 1.60 min; MS (ES+) m/z 584; ¹H NMR δ_{H} (360 MHz, CDCl₃) 8.40 (1H, s), 7.47 (2H, d), 7.24 (1H, t), 7.03 (2H, d), 6.86 (1H, m), 6.69-6.76 (2H, m), 4.55 (1H, br s), 3.93 (3H, s), 3.86-4.02 (2H, obs m), 3.80 (3H, s), 3.70 (1H, dd), 3.61 (1H, m), 3.46 (1H, br s), 2.58-2.99 (3H, m), 2.54 (3H, q), 2.49 (1H, app td), 2.14 (1H, dd), 1.38 (3H, d). For 146, 36% de; LC/MS t_{R} 1.60 min; MS (ES+) m/z 584; ¹H NMR δ_{H} (360 MHz, CDCl₃) 8.39 (1H, s), 7.47 (2H, d), 7.25 (1H, t), 7.03 (2H, d), 6.86 (1H, m), 6.69-6.76 (2H, m), 4.45 (1H, br s), 3.98 (1H, t), 3.93 (3H, s), 3.80 (3H, s), 3.80 (1H, obs m), 3.71 (1H, dd), 3.60 (1H, m), 3.34 (1H, br s), 2.60-2.85 (3H, m), 2.54 (3H, q), 2.40 (1H, m), 1.98 (1H, app td), 1.44 (3H, d).

### Example 140

### [(2R)-4-[(1R)-2-Hydroxy-1-(3-methoxyphenyl)ethyl]-2-methyl-1-piperazinyl][5-(4-methoxyphenyl)-6-methyl-7-(trifluoromethyl)pyrazolo[1,5-a]pyrimidin-3-yl]methanone (147) and [(2R)-4-[(1S)-2-Hydroxy-1-(3-methoxyphenyl)ethyl]-2-methyl-1-piperazinyl][5-(4-methoxyphenyl)-6-methyl-7-(trifluoromethyl)pyrazolo[1,5-a]pyrimidin-3-yl]methanone (148)

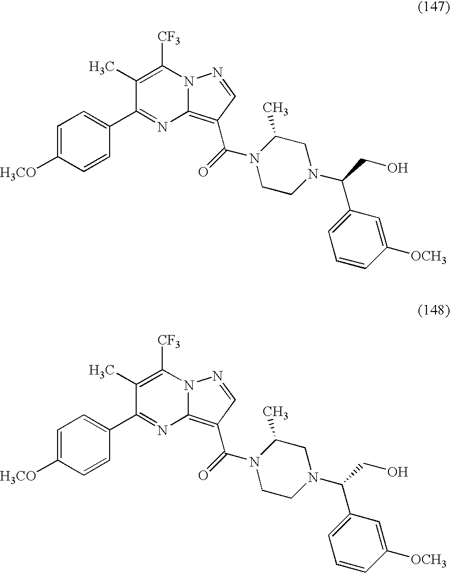

Piperazine 69 (100 mg, 0.23 mmol), glycolaldehyde dimer (15 mg, 0.13 mmol) and 3-methoxyphenylboronic acid (19 mg, 0.13 mmol) were reacted in accordance with Method V and purified by column chromatography (silica gel, 50-70% EtOAc in heptanes) to provide the title diastereomers as vitreous yellow solids: for 147, 30% de; LC/MS t_{R} 1.63 min; MS (ES+) m/z 584; ¹H NMR δ_{H} (360 MHz, CDCl₃) 8.38 (1H, s), 7.47 (2H, d), 7.28 (1H, t), 7.10 (1H, br s), 7.01 (2H, d), 6.91 (2H, m), 4.60 (1H, br s), 4.33 (1H, br s), 3.92 (3H, s), 3.85-4.02 (2H, m), 3.76 (3H, s), 3.59 (1H, m), 3.41 (1H, br s), 2.62-3.01 (2H, m), 2.55 (1H, obs m), 2.53 (3H, q), 2.39 (1H, m), 2.11 (1H, dd), 1.39 (3H, d). For 148, 36% de; LC/MS t_{R} 1.63 min; MS (ES+) m/z 584; ¹H NMR δ_{H} (360 MHz, CDCl₃) 8.38 (1H, s), 7.47 (2H, d), 7.28 (1H, t), 7.10 (1H, br s), 7.01 (2H, d), 6.91 (2H, m), 4.54 (1H, br s), 4.32 (1H, br s), 3.99 (1H, t), 3.93 (3H, s), 3.91 (1H, obs m), 3.76 (3H, s), 3.59 (1H, m), 3.39 (1H, br s), 2.67-3.00 (2H, m), 2.56 (1H, obs m), 2.53 (3H, q), 2.39 (1H, m), 1.95 (1H, app td), 1.45 (3H, d).

### Example 141

### [(2R)-4-[(1S)-2-Hydroxy-1-(5-methyl-2-thienyl)ethyl]-2-methyl-1-piperazinyl][5-(4-methoxyphenyl)-6-methyl-7-(trifluoromethyl)pyrazolo[1,5-a]pyrimidin-3-yl]methanone (149) and [(2R)-4-[(1R)-2-Hydroxy-1-(5-methyl-2-thienyl)ethyl]-2-methyl-1-piperazinyl][5-(4-methoxyphenyl)-6-methyl-7-(trifluoromethyl)pyrazolo[1,5-a]pyrimidin-3-yl]methanone (150)

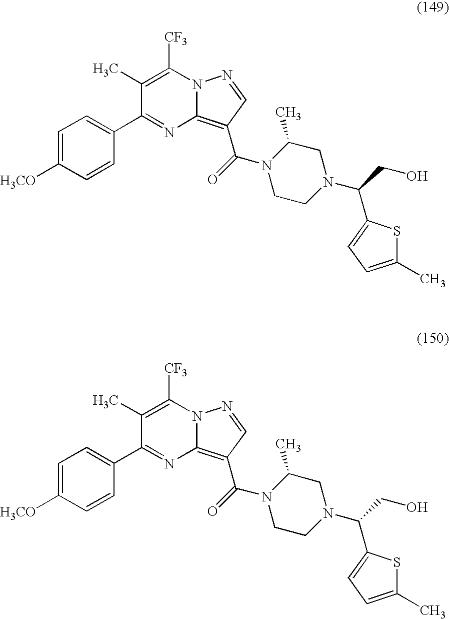

Piperazine 69 (50 mg, 0.12 mmol), glycolaldehyde dimer (8 mg, 0.06 mmol) and 5-methyl-2-thienylboronic acid (18 mg, 0.13 mmol) were reacted in accordance with Method V to afford the title compounds after reverse phase preparative HPLC (5-95% MeCN in water) as vitreous yellow solids: for 149, >95% de; LC/MS t_{R} 1.65 min; MS (ES+) m/z 574; ¹H NMR δ_{H} (360 MHz, CD₃OD) 8.39 (1H, s), 7.61 (2H, d), 7.14 (2H, d), 6.70 (1H, d), 6.66 (1H, d), 4.26 (1H, br s), 3.96 (3H, s), 3.40-3.94 (4H, m), 2.96 (1H, br s), 2.76 (1H, br s), 2.57 (3H, q), 2.46 (3H, s), 2.28-2.53 (3H, m), 1.41 (3H, d). For 150, >95% de; LC/MS t_{R} 1.71 min; MS (ES+) m/z 574; ¹H NMR δ_{H} (360 MHz, CD₃OD) 8.39 (1H, s), 7.60 (2H, d), 7.14 (2H, d), 6.70 (1H, d), 6.66 (1H, d), 4.30 (1H, br s), 3.96 (3H, s), 3.74-4.03 (4H, m), 3.39-3.62 (1H, m), 2.61-3.00 (3H, m), 2.57 (3H, q), 2.46 (3H, s), 2.28 (1H, m), 1.45 (3H, d).

### Example 142

### 5-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]-2-thiophenecarboxylic Acid (151) and 5-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]-2-thiophenecarboxylic Acid (152)

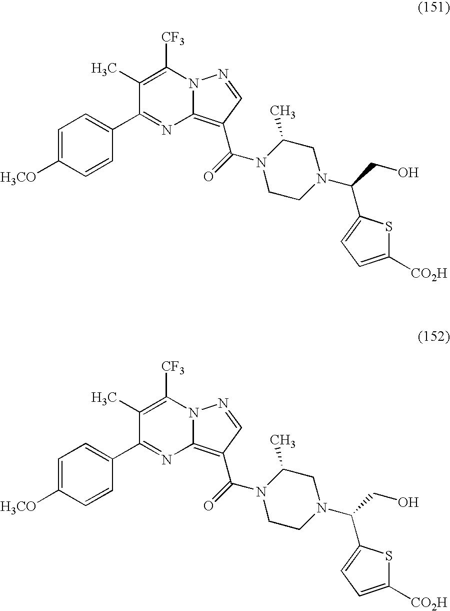

Piperazine 69 (50 mg, 0.12 mmol), glycolaldehyde dimer (8 mg, 0.06 mmol) and 5-borono-2-thiophenecarboxylic acid (22 mg, 0.13 mmol) were reacted in accordance with Method V save that MeCN was used as the reaction solvent in place of DCM. This afforded the title compounds (1:1 mixture of diastereomers) after reverse phase preparative HPLC (5-95% MeCN in water) as a vitreous yellow solid: LC/MS t_{R} 1.64 and 1.67 min; MS (ES+) m/z 604; ¹H NMR δ_{H} (250 MHz, DMSO-d₆) 8.43 and 8.42 (1H, 2 s), 7.55 (2H, d), 7.11 (2H, d), 7.02 (1H, d), 6.70 (1H, d), 4.58 (1H, br s), 4.29 (1H, br m), 3.88 (3H, s), 3.53-3.95 (4H, m), 2.58-2.93 (3H, m), 2.44 (3H, q), 2.09-2.38 (2H, m), 1.28 and 1.23 (3H, 2 d).

### Example 143

### [(2R)-4-[(1R)-2-Hydroxy-1-(4-methyl-3-thienyl)ethyl]-2-methyl-1-piperazinyl][5-(4-methoxyphenyl)-6-methyl-7-(trifluoromethyl)pyrazolo[1,5-a]pyrimidin-3-yl]methanone (153) and [(2R)-4-[(1R)-2-Hydroxy-1-(4-methyl-3-thienyl)ethyl]-2-methyl-1-piperazinyl][5-(4-methoxyphenyl)-6-methyl-7-(trifluoromethyl)pyrazolo[1,5-a]pyrimidin-3-yl]methanone (154)

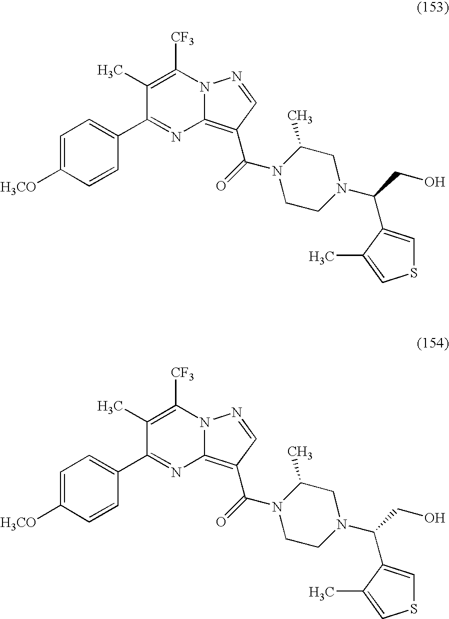

Piperazine 69 (50 mg, 0.12 mmol), glycolaldehyde dimer (8 mg, 0.06 mmol) and 4-methyl-3-thienylboronic acid (18 mg, 0.13 mmol) were reacted in accordance with Method V and purified by column chromatography (silica gel, 0.5% MeOH in DCM) to provide the title diastereomers as vitreous yellow solids: for 153, 28% de; LC/MS t_{R} 1.60 min; MS (ES+) m/z 574; ¹H NMR δ_{H} (360 MHz, CDCl₃) 8.42 (1H, s), 7.47 (2H, d), 7.03 (2H, d), 6.97 (2H, br s), 4.53 (1H, br s), 3.94 (3H, s), 3.84 (2H, m), 3.60 (1H, m), 3.40 (1H, br s), 2.58-3.12 (3H, m), 2.53 (3H, q), 2.44 (1H, app td), 2.28 (1H, dd), 2.18 (3H, br s), 1.39 (3H, d). For 154, 50% de; LC/MS t_{R} 1.62 min; MS (ES+) m/z 574; ¹H NMR δ_{H} (360 MHz, CDCl₃) 8.42 (1H, s), 7.47 (2H, d), 7.03 (2H, d), 6.97 (2H, br s), 4.47 (1H, br s), 3.94 (3H, s), 3.84 (2H, m), 3.61 (1H, m), 3.30 (1H, br s), 2.68-2.99 (2H, m), 2.61 (1H, m), 2.53 (3H, q), 2.23-2.49 (1H, m), 2.18 (3H, br s), 2.13 (1H, app td), 1.41 (3H, d).

### Example 144

### [(2R)-4-[(1R)-1-(2-Chloro-3-pyridinyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (155) and [(2R)-4-[(1S)-1-(2-Chloro-3-pyridinyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (156)

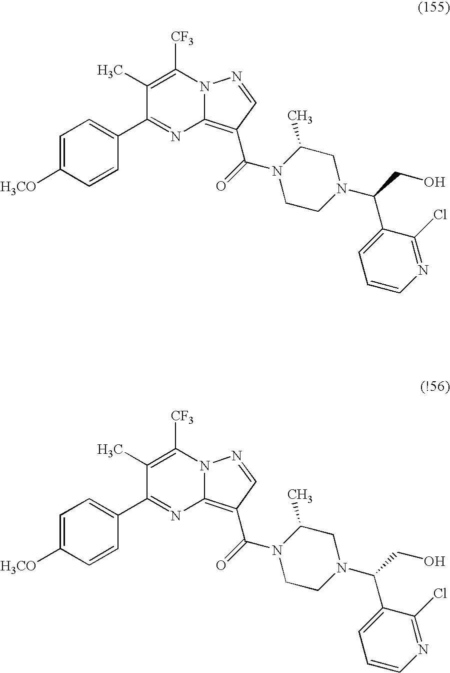

Piperazine 69 (50 mg, 0.12 mmol), glycolaldehyde dimer (8 mg, 0.06 mmol) and 2-chloro-3-pyridinylboronic acid (20 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.91 min; MS (ES+) m/z 589, 591; ¹H NMR δ_{H} (250 MHz, CDCl₃) 8.43 (1H, s), 8.34 (1H, d), 7.75 (1H, m), 7.47 (2H, d), 7.24 (1H, obs m), 7.05 (2H, d), 4.30-4.91 (1H, br m), 4.16 (1H, br m), 3.95 (3H, s), 3.83 (2H, m), 2.98-3.58 (2H, m), 2.68 (1H, m), 2.53 (3H, q), 2.08-2.59 (4H, m), 1.43 and 1.35 (3H, 2 d).

### Example 145

### [(2R)-4-[(1R)-1-(2-Ethoxy-3-pyridinyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (157) and [(2R)-4-[(1S)-1-(2-Ethoxy-3-pyridinyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (158)

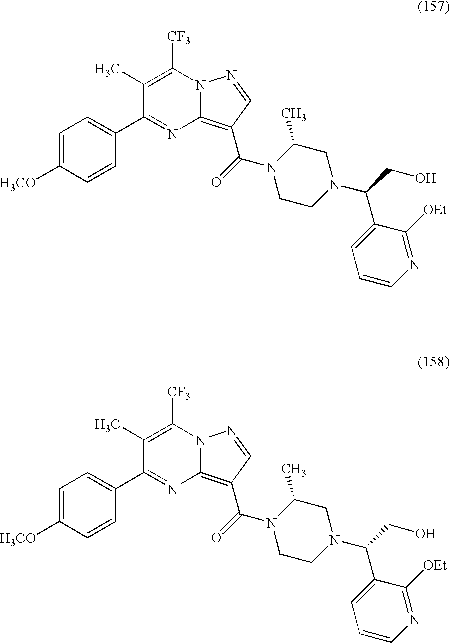

Piperazine 69 (50 mg, 0.12 mmol), glycolaldehyde dimer (8 mg, 0.06 mmol) and 2-ethoxy-3-pyridinylboronic acid (21 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.65 and 1.68 min; MS (ES+) m/z 599; ¹H NMR δ_{H} (250 MHz, CDCl₃) 8.39 (1H, s), 8.09 (1H, dd), 7.47 (2H, d), 7.38 (1H, m), 7.01 (2H, d), 6.82 (1H, m), 4.60 (1H, br s), 4.32 (2H, q), 4.21 (1H, app td), 3.93 (3H, s), 3.91 (1H, obs m), 3.61 (1H, dd), 3.21-3.53 (1H, br m), 2.58-3.12 (3H, m), 2.53 (3H, q), 2.44 (1H, m), 2.14 (0.5H, dd), 1.97 (0.5H, app td), 1.43 and 1.39 (3H, 2 d), 1.34 (3H, t).

### Example 146

### [(2R)-4-[(1R)-2-Hydroxy-1-(6-methoxy-3-pyridinyl)ethyl]-2-methyl-1-piperazinyl][5-(4-methoxyphenyl)-6-methyl-7-(trifluoromethyl)pyrazolo[1,5-a]pyrimidin-3-yl]methanone (159) and [(2R)-4-[(1S)-2-Hydroxy-1-(6-methoxy-3-pyridinyl)ethyl]-2-methyl-1-piperazinyl][5-(4-methoxyphenyl)-6-methyl-7-(trifluoromethyl)pyrazolo[1,5-a]pyrimidin-3-yl]methanone (160)

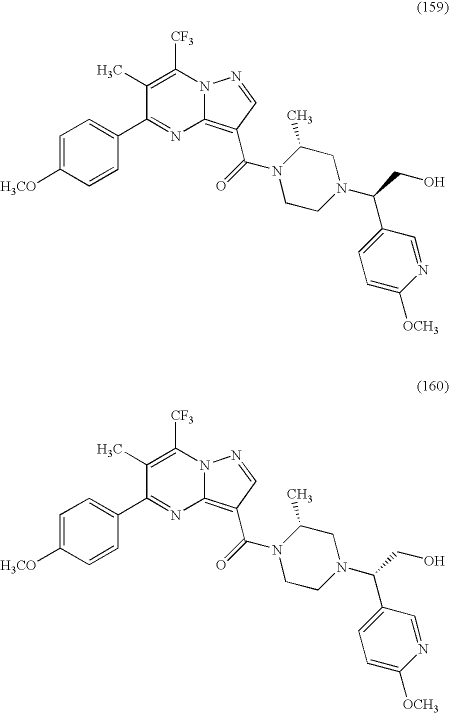

Piperazine 69 (50 mg, 0.12 mmol), glycolaldehyde dimer (8 mg, 0.06 mmol) and 6-methoxy-3-pyridinylboronic acid (20 mg, 0.13 mmol) were reacted in accordance with Method V to afford a 1:1 mixture of the title compounds after column chromatography (silica gel, 60-80% EtOAc in heptanes) as a vitreous yellow solid: LC/MS t_{R} 1.61 min; MS (ES+) m/z 585, 607; ¹H NMR δ_{H} (250 MHz, CDCl₃) 8.41 (1H, s), 7.96 (1H, d), 7.47 (2H, d), 7.37 (1H, dd), 7.07 (2H, d), 6.70 (1H, d), 4.07-4.82 (1H, br m), 3.94 (3H, s), 3.93 (3H, s), 3.90 (1H, obs m), 3.64 (2H, m), 3.16-3.52 (1H, br m), 2.59-2.90 (2H, m), 2.53 (3H, q), 2.18-2.59 (3H, m), 2.12 (0.5H, dd), 1.97 (0.5H, dd), 1.42 and 1.37 (3H, 2 d).

### Example 147

### [(2R)-4-[(1R)-1-(2,6-Dimethoxy-3-pyridinyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (161) and [(2R)-4-[(1S)-1-(2,6-Dimethoxy-3-pyridinyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (162)

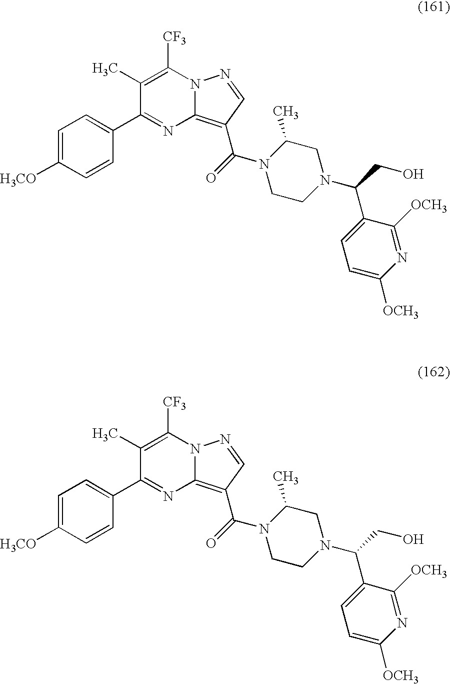

Piperazine 69 (50 mg, 0.12 mmol), glycolaldehyde dimer (8 mg, 0.06 mmol) and 2,6-dimethoxy-3-pyridinylboronic acid (23 mg, 0.13 mmol) were reacted in accordance with Method V to afford a 1:1 mixture of the title compounds after column chromatography (silica gel, 60-80% EtOAc in heptanes) and reverse phase preparative HPLC (5-95% MeCN in water) as a vitreous yellow solid: LC/MS t_{R} 1.63 and 1.65 min; MS (ES+) m/z 615; ¹H NMR δ_{H} (250 MHz, CDCl₃) 8.38 (1H, s), 7.46 (2H, d), 7.28 (1H, obs d), 7.01 (2H, d), 6.25 (1H, d), 4.59 (1H, br s), 4.11 (1H, br s), 3.91 (6H, s), 3.88 (3H, s), 3.75-4.00 (2H, obs m), 3.54 (1H, m), 3.39 (1H, br s), 2.93 (1H, m), 2.77 (1H, m), 2.52 (3H, q), 2.32-2.66 (2H, m), 2.11 (0.5H, dd), 1.95 (0.5H, app td), 1.42 and 1.37 (3H, 2 d).

### Example 148

### [(2R)-4-[(1R)-2-Hydroxy-1-(2-methoxy-5-pyrimidinyl)ethyl]-2-methyl-1-piperazinyl][5-(4-methoxyphenyl)-6-methyl-7-(trifluoromethyl)pyrazolo[1,5-a]pyrimidin-3-yl]methanone (163) and [(2R)-4-[(1S)-2-Hydroxy-1-(2-methoxy-5-pyrimidinyl)ethyl]-2-methyl-1-piperazinyl][5-(4-methoxyphenyl)-6-methyl-7-(trifluoromethyl)pyrazolo[1,5-a]pyrimidin-3-yl]methanone (164)

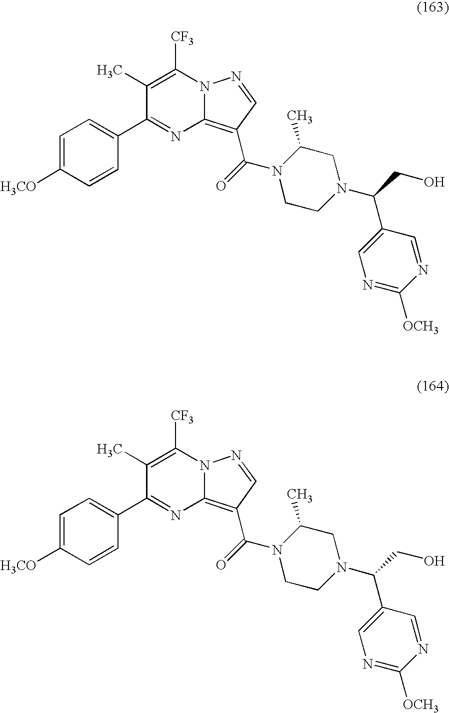

Piperazine 69 (50 mg, 0.12 mmol), glycolaldehyde dimer (8 mg, 0.06 mmol) and 2-methoxy-5-pyrimidinylboronic acid (20 mg, 0.13 mmol) were reacted in accordance with Method V to afford a 1:1 mixture of the title compounds after reverse phase preparative HPLC (5-95% MeCN in water) and column chromatography (silica gel, 0-2% MeOH in DCM) as a vitreous yellow solid: LC/MS t_{R} 1.66 min; MS (ES+) m/z 586, 608; ¹H NMR δ_{H} (360 MHz, CDCl₃) 8.44 and 8.43 (1H, 2 s), 8.36 (2H, s), 7.47 (2H, d), 7.10 (2H, d), 4.71 (1H, br m), 4.03 (3H, s), 3.96 (3H, s), 3.95 (1H, obs m), 3.75 (1H, m), 3.63 (1H, ddd), 3.21-3.55 (1H, m), 2.54 (3H, q), 2.39-2.97 (4H, m), 2.21 (0.5H, dd), 2.05 (0.5H, app td), 1.42 and 1.38 (3H, 2 d).

### Example 149

### N-[3-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]acetamide (165) and N-[3-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]acetamide (166)

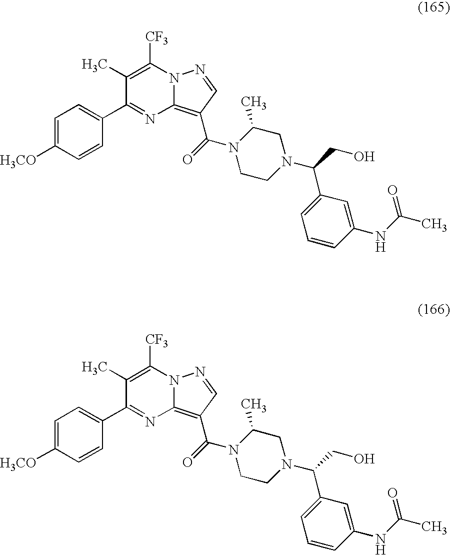

Piperazine 69 (50 mg, 0.12 mmol), glycolaldehyde dimer (8 mg, 0.06 mmol) and 3-(acetylamino)phenylboronic acid (23 mg, 0.13 mmol) were reacted in accordance with Method V to afford a 5:4 mixture of the title compounds after reverse phase preparative HPLC (5-95% MeCN in water) as a vitreous yellow solid: LC/MS t_{R} 1.52 min; MS (ES+) m/z 611, 633; ¹H NMR δ_{H} (250 MHz, CDCl₃) 8.38 and 8.37 (1H, 2 s), 7.58 (1H, s), 7.32-7.51 (4H, m), 7.24 (1H, obs t), 7.01 (2H, d), 6.87 (1H, d), 4.04-4.87 (1H, br m), 3.92 (3H, s), 3.91 (1H, obs m), 3.70 (1H, m), 3.59 (1H, m), 3.43 (1H, br m), 2.53 (3H, q), 2.23-2.93 (4H, m), 2.15 (3H, s), 2.10 (0.5H, obs dd), 1.95 (0.5H, t), 1.43 and 1.37 (3H, 2 d).

### Example 150

### [(2R)-4-[(1R)-2-Hydroxy-1-[3-(methylsulfonyl)phenyl]ethyl]-2-methyl-1-piperazinyl][5-(4-methoxyphenyl)-6-methyl-7-(trifluoromethyl)pyrazolo[1,5-a]pyrimidin-3-yl]methanone (167) and [(2R)-4-[(1S)-2-Hydroxy-1-[3-(methylsulfonyl)phenyl]ethyl]-2-methyl-1-piperazinyl][5-(4-methoxyphenyl)-6-methyl-7-(trifluoromethyl)pyrazolo[1,5-a]pyrimidin-3-yl]methanone (168)

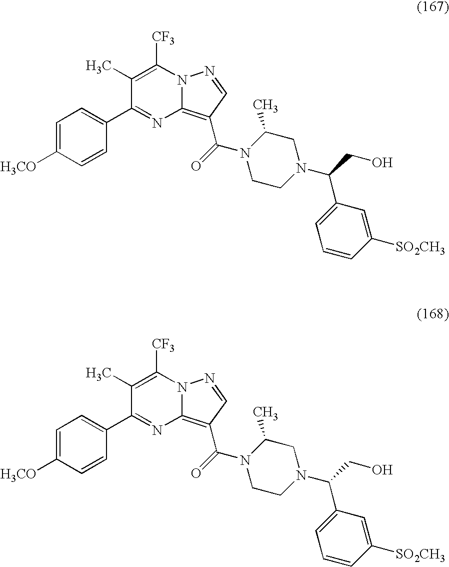

Piperazine 69 (50 mg, 0.12 mmol), glycolaldehyde dimer (8 mg, 0.06 mmol) and 3-(methylsulfonyl)phenylboronic acid (25 mg, 0.13 mmol) were reacted in accordance with Method V to afford a 5:4 mixture of the title compounds after reverse phase preparative HPLC (5-95% MeCN in water) as a vitreous yellow solid: LC/MS t_{R} 1.68 min; MS (ES+) m/z 632, 654; ¹H NMR δ_{H} (250 MHz, CDCl₃) 8.41 (1H, s), 7.88 (1H, d), 7.79 (1H, d), 7.55 (1H, t), 7.51 (1H, m), 7.47 (2H, d), 7.04 (2H, d), 4.08-4.82 (1H, br m), 3.93 (3H, s), 3.89-4.01 (1H, obs m), 3.78 (1H, m), 3.70 (1H, m), 3.46 (1H, br m), 3.05 (3H, s), 2.98 (1H, m), 2.78 (1H, app td), 2.53 (3H, q), 2.36-2.65 (3H, m), 2.12 (0.5H, dd), 1.98 (0.5H, app td), 1.46 and 1.39 (3H, 2 d).

### Example 151

### [(2R)-4-[(1R)-1-(3,5-Dimethyl-4-isoxazolyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (169) and [(2R)-4-[(1S)-1-(3,5-Dimethyl-4-isoxazolyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (170)

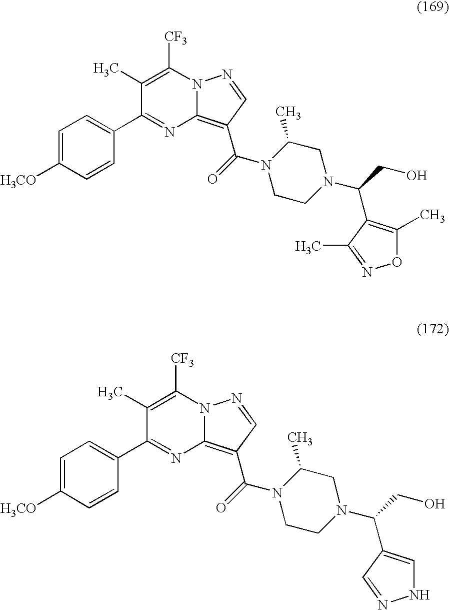

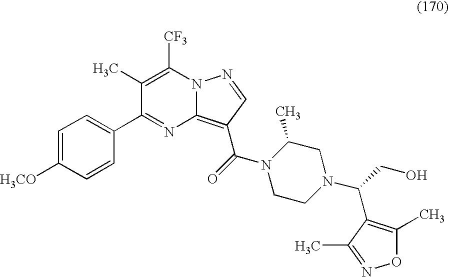

Piperazine 69 (50 mg, 0.12 mmol), glycolaldehyde dimer (8 mg, 0.06 mmol) and 3,5-dimethyl-4-isoxazolylboronic acid (18 mg, 0.13 mmol) were reacted in accordance with Method V to afford the title compounds after reverse phase preparative HPLC (5-95% MeCN in water) and column chromatography (SCX silica gel, MeOH then NH₃ in MeOH, 2 M) as vitreous yellow solids: for 169, 26% de; LC/MS t_{R} 1.73 min; MS (ES+) m/z 573; ¹H NMR δ_{H} (360 MHz, CDCl₃) 8.44 (1H, s), 7.46 (2H, d), 7.03 (2H, d), 4.49 (1H, br s), 3.95 (3H, s), 3.93 (1H, obs m), 3.65 (1H, m), 3.44 (2H, m), 2.67-3.01 (1H, br m), 2.54 (3H, q), 2.23-2.58 (3H, m), 2.29 (3H, s), 2.21 (3H, s), 2.19 (1H, obs dd), 1.34 (3H, d). For 170, 74% de; LC/MS t_{R} 1.83 min; MS (ES+) m/z 573; ¹H NMR δ_{H} (360 MHz, CDCl₃) 8.45 (1H, s), 7.46 (2H, d), 7.03 (2H, d), 4.26-4.80 (1H, br m), 3.96 (3H, s), 3.94 (1H, obs m), 3.65 (1H, m), 3.44 (1H, m), 3.30 (1H, br s), 2.54 (3H, q), 2.51-2.90 (3H, br m), 2.29 (3H, s), 2.24-2.38 (1H, m), 2.21 (3H, s), 2.06 (1H, app td), 1.41 (3H, d).

### Example 152

### [(2R)-4-[(1R)-2-Hydroxy-1-(1H-pyrazol-4-yl)ethyl]-2-methyl-1-piperazinyl][5-(4-methoxyphenyl)-6-methyl-7-(trifluoromethyl)pyrazolo[1,5-a]pyrimidin-3-yl]methanone (171) and [(2R)-4-[(1S)-2-Hydroxy-1-(1H-pyrazol-4-yl)ethyl]-2-methyl-1-piperazinyl][5-(4-methoxyphenyl)-6-methyl-7-(trifluoromethyl)pyrazolo[1,5-a]pyrimidin-3-yl]methanone (172)

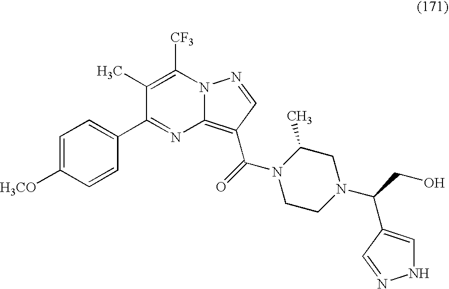

Piperazine 69 (50 mg, 0.12 mmol), glycolaldehyde dimer (8 mg, 0.06 mmol) and 1H-pyrazolyl-4-boronic acid (15 mg, 0.13 mmol) were reacted in accordance with Method V save that MeCN was used as the reaction solvent in place of DCM. This afforded the title compounds (1:1 mixture of diastereomers) after column chromatography (silica gel, 0-10% MeOH in DCM) as a vitreous yellow solid: LC/MS t_{R} 1.40 min; MS (ES+) m/z 544; ¹H NMR δ_{H} (360 MHz, CDCl₃) 8.42 (1H, s), 7.46 (2H, d), 7.36 (2H, s), 7.06 (2H, d), 4.10-5.03 (2H, br m), 3.95 (3H, s), 3.72-3.92 (3H, m), 3.62 (1H, m), 3.36 (1H, br m), 2.54 (3H, q), 2.43-2.82 (3H, m), 2.10 (0.5H, dd), 1.94 (0.5H, app td), 1.42 and 1.39 (3H, 2 d).

### Example 153

### [(2R)-4-[(1R)-1-(6-Chloro-3-pyridinyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (173) and [(2R)-4-[(1S)-1-(6-Chloro-3-pyridinyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (174)

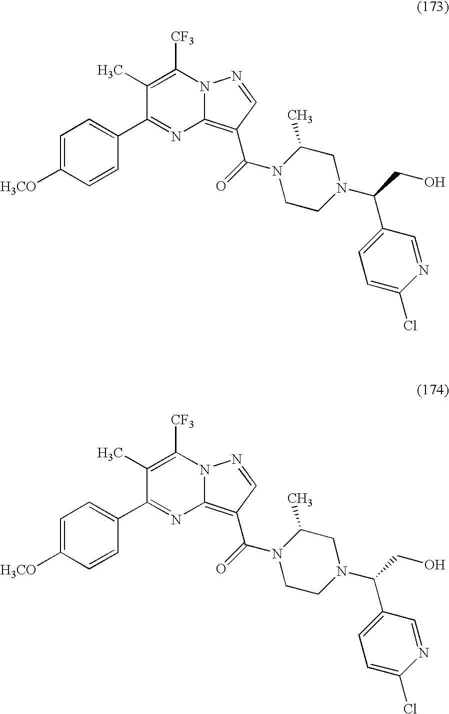

Piperazine 69 (50 mg, 0.12 mmol), glycolaldehyde dimer (8 mg, 0.06 mmol) and 6-chloro-3-pyridinylboronic acid (20 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.82 min; MS (ES+) m/z 589, 591; ¹H NMR δ_{H} (250 MHz, CDCl₃) 8.43 (1H, s), 8.22 (1H, d), 7.50 (1H, dd), 7.46 (2H, d), 7.30 (1H, d), 7.06 (2H, d), 4.05-4.86 (2H, br m), 3.97 (3H, s), 3.91 (1H, m), 3.75 (1H, m), 3.63 (1H, m), 3.18-3.54 (1H, br m), 2.54 (3H, q), 2.35-3.03 (4H, m), 2.16 (0.5H, dd), 2.01 (0.5H, app td), 1.42 and 1.37 (3H, 2 d).

### Example 154

### [(2R)-4-[(1R)-2-Hydroxy-1-(4-methoxy-3-pyridinyl)ethyl]-2-methyl-1-piperazinyl][5-(4-methoxyphenyl)-6-methyl-7-(trifluoromethyl)pyrazolo[1,5-a]pyrimidin-3-yl]methanone (175) and [(2R)-4-[(1S)-2-Hydroxy-1-(4-methoxy-3-pyridinyl)ethyl]-2-methyl-1-piperazinyl][5-(4-methoxyphenyl)-6-methyl-7-(trifluoromethyl)pyrazolo[1,5-a]pyrimidin-3-yl]methanone (176)

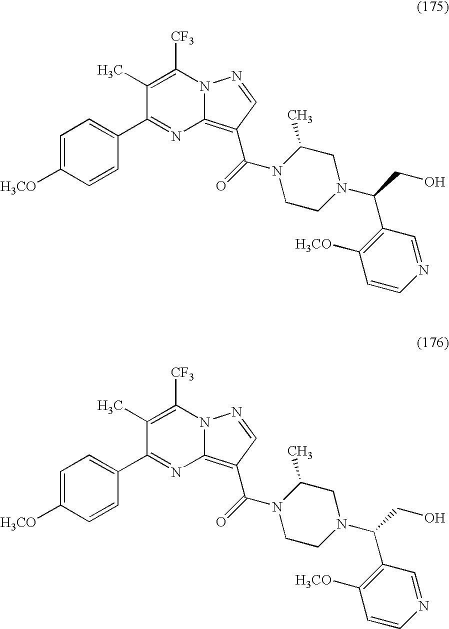

Piperazine 69 (50 mg, 0.12 mmol), glycolaldehyde dimer (8 mg, 0.06 mmol) and 4-methoxy-3-pyridinylboronic acid (20 mg, 0.13 mmol) were reacted in accordance with Method V to afford a 1:1 mixture of the title compounds after reverse phase preparative HPLC (5-95% MeCN in water) and column chromatography (SCX silica gel, MeOH then NH₃ in MeOH, 2 M) as a vitreous yellow solid: LC/MS t_{R} 1.60 min; MS (ES+) m/z 585; ¹H NMR δ_{H} (360 MHz, CDCl₃) 8.42 (1H, s), 7.97 (1H, d), 7.46 (2H, d), 7.37 (1H, dd), 7.08 (2H, d), 6.71 (1H, d), 4.60 (1H, br s), 3.95 (6H, s), 3.94 (1H, obs m), 3.65 (2H, m), 3.21-3.47 (1H, br m), 2.54 (3H, q), 2.43-3.01 (4H, m), 2.13 (0.5H, dd), 1.98 (0.5H, app td), 1.43 and 1.40 (3H, 2 d).

### Example 155

### [(2R)-4-[(1R)-1-(6-Ethoxy-3-pyridinyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (177) and [(2R)-4-[(1S)-1-(6-Ethoxy-3-pyridinyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (178)

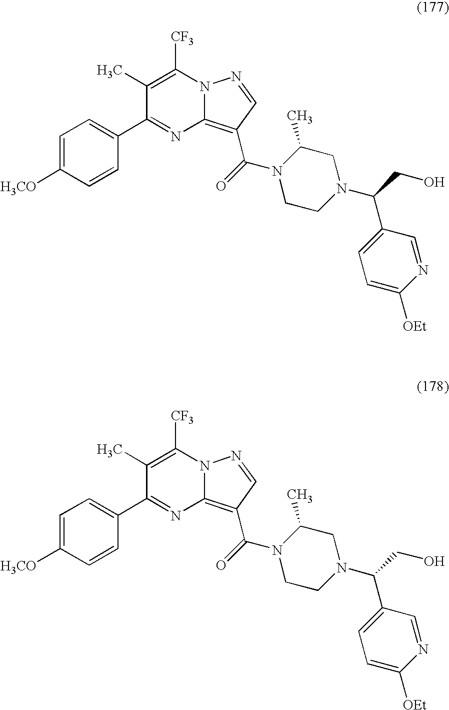

Piperazine 69 (50 mg, 0.12 mmol), glycolaldehyde dimer (8 mg, 0.06 mmol) and 6-ethoxy-3-pyridinylboronic acid (21 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.66 and 1.68 min; MS (ES+) m/z 599, 621; ¹H NMR δ_{H} (250 MHz, CDCl₃) 8.40 (1H, s), 7.93 (1H, d), 7.46 (2H, d), 7.36 (1H, dd), 7.07 (2H, d), 6.67 (1H, d), 4.62 (1H, br s), 4.34 (2H, q), 4.05-4.42 (1H, br m), 3.94 (3H, s), 3.92 (1H, obs m), 3.63 (2H, m), 3.36 (1H, br m), 2.53 (3H, q), 2.32-3.08 (4H, m), 2.12 (0.5H, dd), 1.96 (0.5H, app td), 1.40 and 1.39 (3H, 2 obs d).

### Example 156

### 3-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]benzenesulfonamide (179) and 3-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]benzenesulfonamide (180)

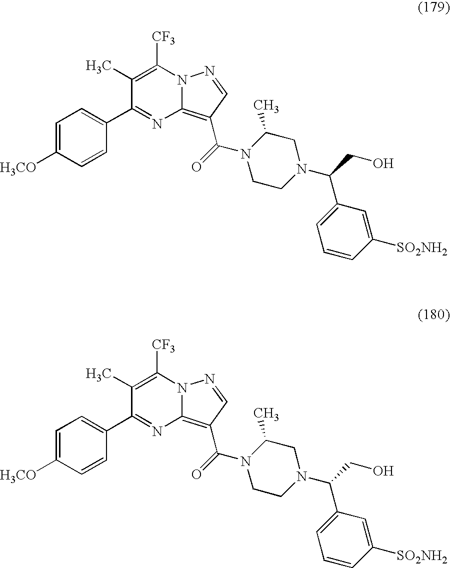

Piperazine 69 (50 mg, 0.12 mmol), glycolaldehyde dimer (8 mg, 0.06 mmol) and 3-(aminosulfonyl)phenylboronic acid (26 mg, 0.13 mmol) were reacted in accordance with Method V to afford a 1:1 mixture of the title compounds after reverse phase preparative HPLC (5-95% MeCN in water) as a powdery yellow solid: LC/MS t_{R} 1.59 min; MS (ES+) m/z 633, 655; ¹H NMR δ_{H} (250 MHz, CD₃OD) 8.38 and 8.37 (1H, 2 s), 7.90 (1H, d), 7.81 (1H, d), 7.44-7.65 (4H, m), 7.10 (2H, d), 3.92 (3H, s), 3.89 (3H, obs m), 3.56 (2H, s), 3.54 (1H, br m), 3.17 (1H, m), 3.03 (1H, m), 2.83 (1H, dd), 2.55 (1H, obs m), 2.52 (3H, q), 2.19 (1H, m), 1.83-1.99 (1H, m), 1.44 and 1.35 (3H, 2 d).

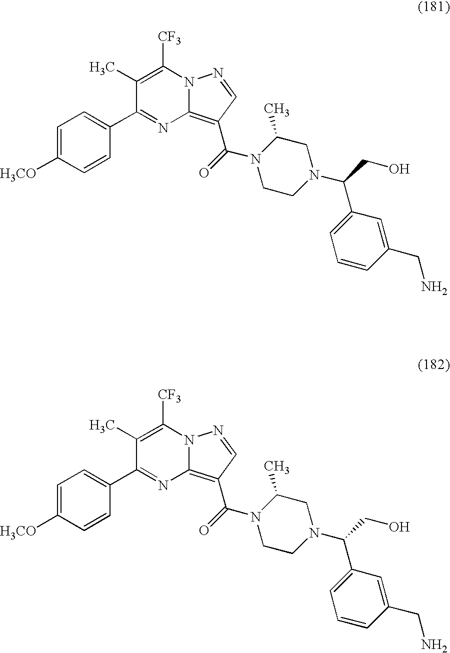

### Example 157

### N-[[3-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]methyl]carbamic Acid, 1,1-Dimethylethyl Ester (183) and N-[[3-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]methyl]carbamic Acid, 1,1-Dimethylethyl Ester (184)

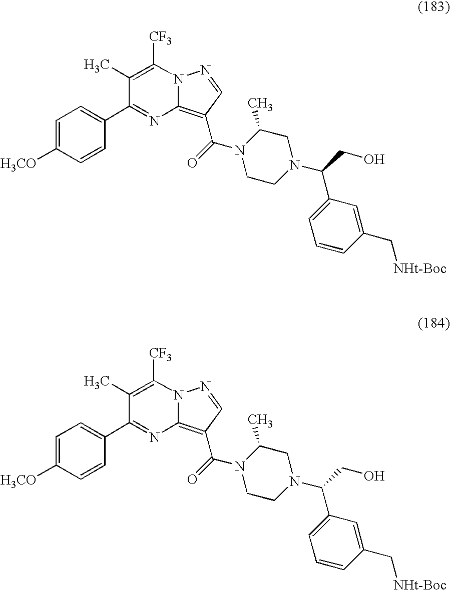

Piperazine 69 (50 mg, 0.12 mmol), glycolaldehyde dimer (8 mg, 0.06 mmol) and 3-[(tert-butoxycarbonyl)aminomethyl]phenylboronic acid (32 mg, 0.13 mmol) were reacted in accordance with Method V to afford a 3:2 mixture of the title compounds after reverse phase preparative HPLC (5-95% MeCN in water) as a vitreous yellow solid: LC/MS t_{R} 1.73 and 1.76 min; MS (ES+) m/z 683, 705.

### Example 158

### [(2R)-4-[(1R)-1-[3-(Aminomethyl)phenyl]-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (181) and [(2R)-4-[(1S)-1-[3-(Aminomethyl)phenyl]-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (182)

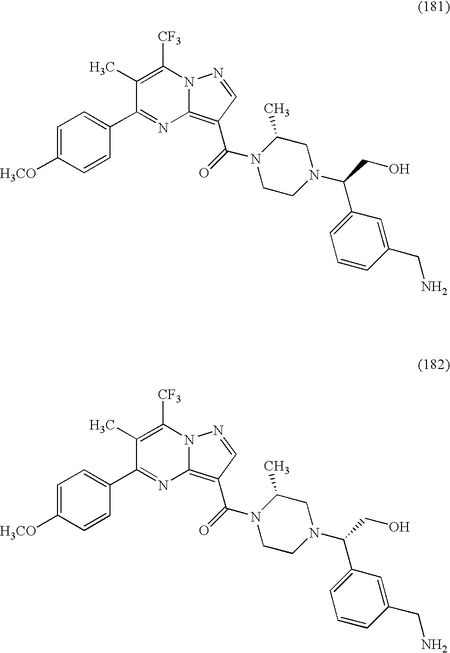

The 3:2 mixture of tert-butyl carbamates 183 and 184 (31 mg, 0.045 mmol) was treated with TFA in accordance with Method O. On completion, the reaction mixture was reduced in vacuo to afford a 3:2 mixture of the TFA salts of the title compounds as a glassy orange solid: LC/MS t_{R} 1.29 min; MS (ES+) m/z 292, 312.5, 583; ¹H NMR δ_{H} (250 MHz, CD₃OD) 8.44 and 8.42 (1H, 2 s), 7.26-7.71 (6H, m), 7.15 and 7.13 (2H, 2 d), 4.98-5.28 (1H, br m), 4.55 (1H, dd), 4.15 (2H, s), 4.00-4.51 (3H, m), 3.94 (3H, s), 3.91 (1H, obs m), 3.53-3.79 (1H, m), 3.42 (1H, br d), 2.83-3.28 (2H, m), 2.50 (3H, q), 1.54 and 1.46 (3H, 2 d).

### Example 159

### N-[[3-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]methyl]acetamide (185) and N-[[3-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]methyl]acetamide (186)

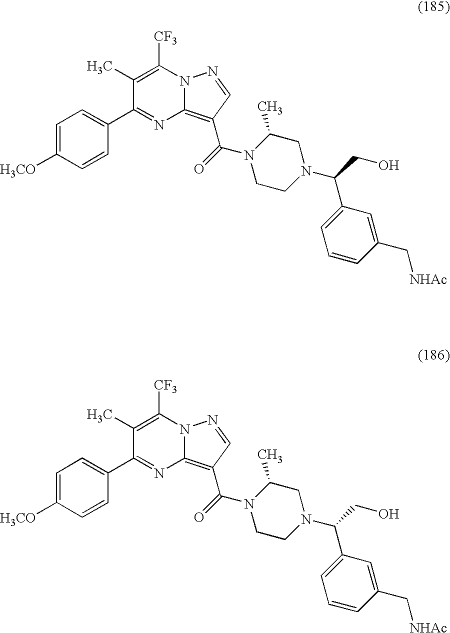

Piperazine 69 (50 mg, 0.12 mmol), glycolaldehyde dimer (8 mg, 0.06 mmol) and 3-[(acetylamino)methyl]phenylboronic acid (25 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.48 min; MS (ES+) m/z 625, 647; ¹H NMR δ_{H} (360 MHz, CDCl₃) 8.37 and 8.36 (1H, 2 s), 7.46 (2H, d), 7.27 (2H, obs m), 7.05 (2H, obs m), 7.02 (2H, d), 5.96 (1H, br s), 4.38 (1H, d), 4.37 (1H, d), 4.06-4.90 (1H, br m), 3.92 (3H, s), 3.84-4.02 (2H, obs m), 3.68 (1H, dd), 3.61 (1H, m), 3.39 (1H, br m), 2.89 (1H, br s), 2.74 (1H, br d), 2.53 (3H, q), 2.38-2.62 (2H, m), 2.06 (0.5H, dd), 1.91 (0.5H, app td), 2.02 (3H, s), 1.44 and 1.38 (3H, 2 d).

### Example 160

### 3-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]benzamide (187) and 3-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]benzamide (188)

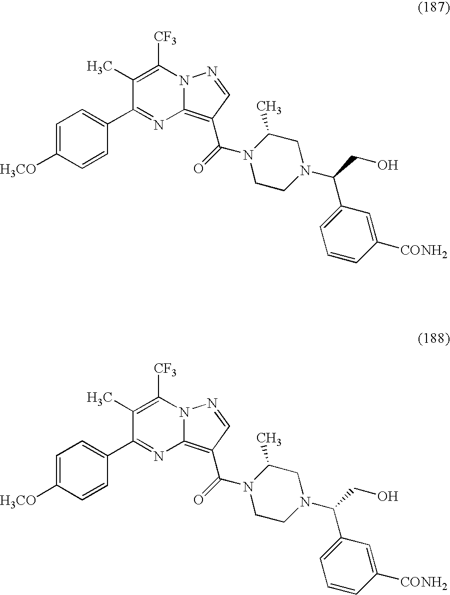

Piperazine 69 (50 mg, 0.12 mmol), glycolaldehyde dimer (8 mg, 0.06 mmol) and 3-(aminocarbonyl)phenylboronic acid (21 mg, 0.13 mmol) were reacted in accordance with Method V save that MeCN was used as the reaction solvent in place of DCM. This afforded a 1:1 mixture of the title compounds after column chromatography (silica gel, 0-10% MeOH in DCM) as a vitreous yellow solid: LC/MS t_{R} 1.49 min; MS (ES+) m/z 597; ¹H NMR δ_{H} (250 MHz, CDCl₃) 8.40 and 8.39 (1H, 2 s), 7.69 (2H, br s), 7.48 (1H, obs m), 7.46 (2H, d), 7.29-7.53 (2H, obs m), 7.05 (1H, obs m), 7.02 (2H, d), 4.10-4.95 (1H, br m), 3.94 (3H, s), 3.84-4.05 (2H, obs m), 3.72 (2H, m), 3.40 (1H, br m), 2.53 (3H, q), 2.25-3.03 (4H, m), 2.09 (0.5H, dd), 1.94 (0.5H, app td), 1.45 and 1.39 (3H, 2 d).

### Example 161

### [(2R)-4-[(1R)-1-(6-Bromo-3-pyridinyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (189) and [(2R)-4-[(1S)-1-(6-Bromo-3-pyridinyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (190)

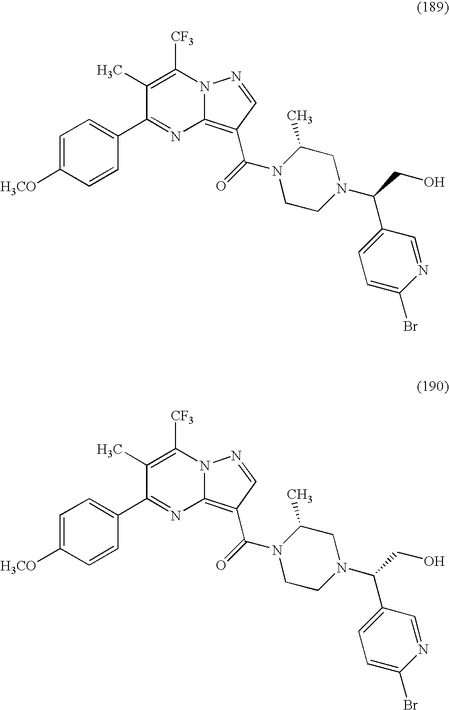

Piperazine 69 (50 mg, 0.12 mmol), glycolaldehyde dimer (8 mg, 0.06 mmol) and 6-bromo-3-pyridinylboronic acid (26 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.83 min; MS (ES+) m/z 633, 635; ¹H NMR δ_{H} (250 MHz, CDCl₃) 8.42 (1H, s), 8.20 (1H, s), 7.46 (2H, d), 7.34-7.54 (2H, obs m), 7.06 (2H, d), 4.65 (1H, br m), 3.97 (3H, s), 3.90 (1H, obs m), 3.76 (1H, m), 3.61 (1H, m), 3.42 (1H, br m), 2.53 (3H, q), 2.28-2.99 (4H, m), 2.15 (0.5H, dd), 2.01 (0.5H, app td), 1.41 and 1.36 (3H, 2 d).

### Example 162

### 5-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]-N,N-dimethyl-2-thiophenecarboxamide (191) and 5-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]-N,N-dimethyl-2-thiophenecarboxamide (192)

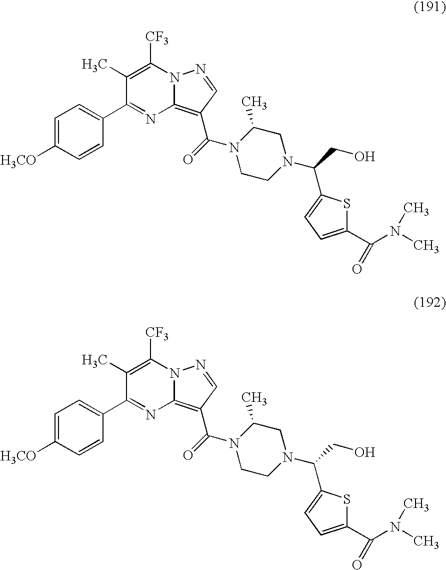

The 1:1 mixture of carboxylic acids 151 and 152 (6.0 mg, 0.01 mmol) was treated with HATU (4.5 mg, 0.012 mmol), DIPEA (6.9 μL, 0.04 mmol, d 0.742) and dimethylamine hydrochloride (2.4 mg, 0.03 mmol) employing the procedure of Method G to give a 1:1 mixture of the title compounds after column chromatography (SCX silica gel, MeOH then NH₃ in MeOH, 2 M) as a vitreous yellow solid: LC/MS t_{R} 1.61 and 1.65 min; MS (ES+) m/z 631, 653; ¹H NMR δ_{H} (250 MHz, CDCl₃) 8.43 and 8.42 (1H, 2 s), 7.48 (2H, d), 7.24 (1H, d), 7.05 (2H, d), 6.77 (1H, d), 4.17-4.93 (1H, br m), 3.92 (3H, s), 3.71-4.14 (4H, m), 3.27-3.49 (1H, br m), 3.18 (6H, br s), 3.05-3.22 (1H, obs m), 2.53 (3H, q), 2.48-2.83 (3H, m), 2.27 (0.5H, dd), 2.10 (0.5H, app td), 1.43 and 1.40 (3H, 2 d).

### Example 163

### [(2R)-4-[(1R)-1-(2-Aminophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (193) and [(2R)-4-[(1S)-1-(2-Aminophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (194)

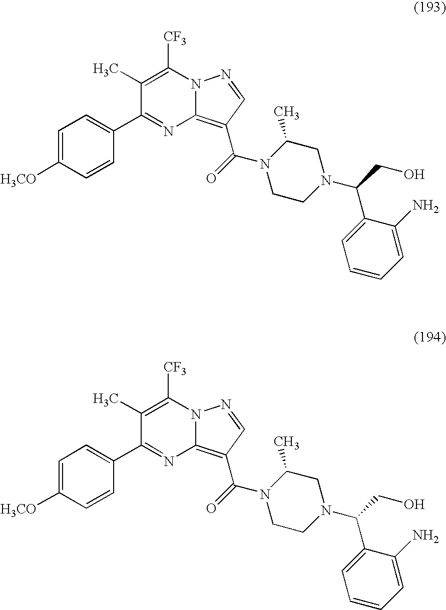

Piperazine 69 (50 mg, 0.12 mmol), glycolaldehyde dimer (8 mg, 0.06 mmol) and 2-aminophenylboronic acid (17 mg, 0.13 mmol) were reacted in accordance with Method V to afford a 1:1 mixture of the title compounds after column chromatography (silica gel, 0-10% MeOH in DCM) as a vitreous yellow solid: LC/MS t_{R} 1.61 min; MS (ES+) m/z 569; ¹H NMR δ_{H} (250 MHz, CDCl₃) 8.452 (1H, 2 s), 7.49 (2H, d), 7.10 (1H, t), 7.03 (2H, d), 6.95 (1H, d), 6.72 (1H, t), 6.64 (1H, d), 4.16-5.12 (3H, br s), 4.05 (1H, m), 3.92 (3H, s), 3.73-4.00 (2H, obs m), 3.07-3.61 (3H, m), 2.54 (3H, q), 2.39-2.84 (2H, m), 2.29 (0.5H, dd), 2.18 (0.5H, app td), 1.40 and 1.32 (3H, 2 d).

### Example 164

### N-[2-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]acetamide (195) and N-[2-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]acetamide (196)

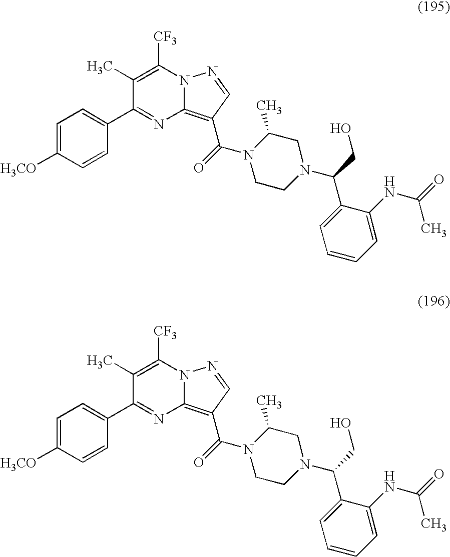

A suspension of a 1:1 mixture of anilines 193 and 194 (22 mg, 0.039 mmol) and NaHCO₃ (4.9 mg, 0.059 mmol) in DCM (2 mL) was cooled to 0° C. and treated with acetyl chloride (3.1 μL, 0.043 mmol, d 1.104). After stirring at rt for 1 h, the reaction mixture was diluted with DCM (10 mL) and water (10 mL) and the two phases separated. The organic phase was washed with water (2×10 mL) and brine (10 mL), dried (Na₂SO₄), filtered and reduced in vacuo. Column chromatography (silica gel, 0-2% MeOH in DCM) afforded a 1:1 mixture of the title compounds as a vitreous yellow solid: LC/MS t_{R} 1.64 and 1.68 min; MS (ES+) m/z 611, 633; ¹H NMR δ_{H} (360 MHz, CDCl₃) 10.59 and 10.51 (1H, 2 br s), 8.46 (1H, s), 8.30 (1H, dd), 7.48 (2H, m), 7.31 (1H, app td), 6.98-7.10 (4H, m), 4.28-5.01 (1H, br m), 3.97 (2H, m), 3.93 (3H, s), 3.90 (1H, obs m), 3.02-3.60 (3H, m), 2.55 (3H, m), 2.43-2.80 (2H, m), 2.16 (3H, s), 2.06-2.40 (2H, m), 1.47 and 1.34 (3H, 2 d).

### Example 165

### N-[2-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]cyclopropanecarboxamide (197) and N-[2-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]cyclopropanecarboxamide (198)

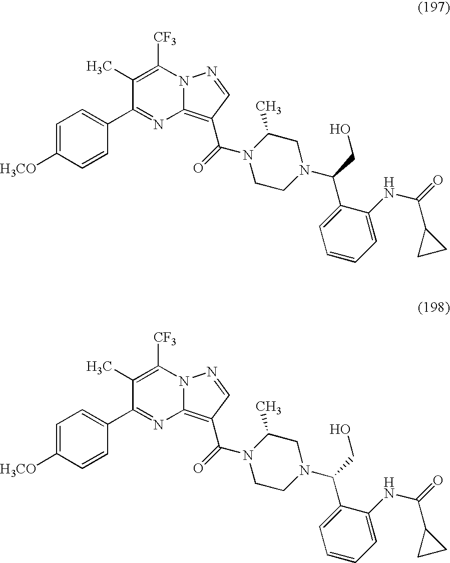

A suspension of a 1:1 mixture of anilines 193 and 194 (20 mg, 0.035 mmol) and NaHCO₃ (4.4 mg, 0.053 mmol) in DCM (2 mL) was cooled to 0° C. and treated with cyclopropanecarbonyl chloride (3.5 μL, 0.039 mmol, d 1.152). After stirring at rt for 1 h, the reaction mixture was diluted with DCM (10 mL) and water (10 mL) and the two phases separated. The organic phase was washed with water (2×10 mL) and brine (10 mL), dried (Na₂SO₄), filtered and reduced in vacuo. Column chromatography (silica gel, 0-2% MeOH in DCM) afforded a 1:1 mixture of the title compounds as a vitreous yellow solid: LC/MS t_{R} 1.77 min; MS (ES+) m/z 637, 659; ¹H NMR δ_{H} (360 MHz, CDCl₃) 10.83 and 10.66 (1H, 2 br s), 8.45 (1H, s), 8.29 (1H, m), 7.47 (2H, m), 7.28 (1H, app td), 6.95-7.09 (4H, m), 4.26-5.05 (1H, br m), 3.99 (2H, br s), 3.93 (3H, s), 3.72-3.95 (1H, obs m), 3.10-3.53 (3H, m), 2.54 (3H, m), 2.12-2.79 (4H, m), 1.48 (1H, obs m), 1.45 and 1.36 (3H, 2 d), 0.98-1.12 (2H, m), 0.81 (2H, m).

### Example 166

### N-[2-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]-2-methylpropaneamide (199) and N-[2-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]-2-methylpropaneamide (200)

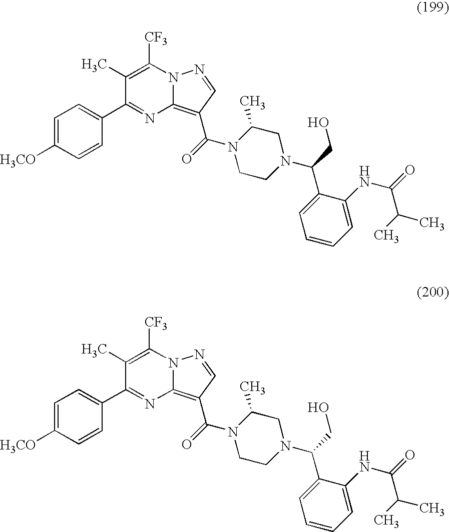

A suspension of a 1:1 mixture of anilines 193 and 194 (20 mg, 0.035 mmol) and NaHCO₃ (4.4 mg, 0.053 mmol) in DCM (2 mL) was cooled to 0° C. and treated with isobutyryl chloride (4.1 μL, 0.039 mmol, d 1.017). After stirring at rt for 1 h, the reaction mixture was diluted with DCM (10 mL) and water (10 mL) and the two phases separated. The organic phase was washed with water (2×10 mL) and brine (10 mL), dried (Na₂SO₄), filtered and reduced in vacuo. Column chromatography (silica gel, 0-2% MeOH in DCM) afforded a 1:1 mixture of the title compounds as a vitreous yellow solid: LC/MS t_{R} 1.78 and 1.83 min; MS (ES+) m/z 639, 661; ¹H NMR δ_{H} (360 MHz, CDCl₃) 10.43 and 10.33 (1H, 2 br s), 8.45 (1H, s), 8.33 and 8.25 (1H, 2 d), 7.47 (2H, m), 7.29 (1H, app td), 6.98-7.09 (4H, m), 4.27-5.05 (1H, br m), 3.92 (3H, s), 3.72-4.02 (3H, obs m), 3.04-3.56 (3H, m), 2.53 (3H, m), 2.15-2.80 (5H, m), 1.44 and 1.31 (3H, 2 d), 1.23 (6H, d).

### Example 167

### N-[2-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]urea (201) and N-[2-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]urea (202

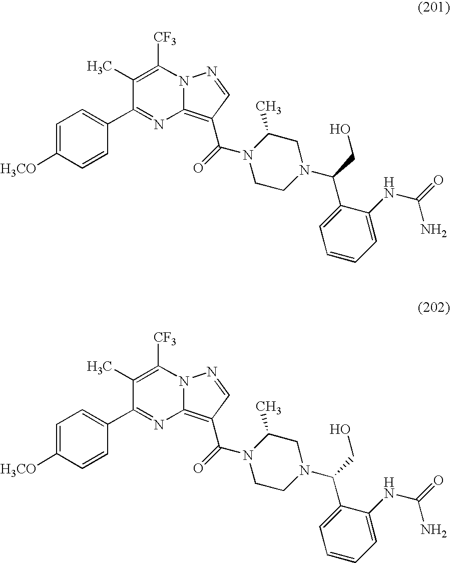

A solution of a 1:1 mixture of anilines 193 and 194 (30 mg, 0.053 mmol) in AcOH (0.5 mL) and water (0.5 mL) was treated with potassium cyanate (26 mg, 0.32 mmol) and stirred at rt for 16 h. Reaction progress was monitored by LC/MS. On completion the reaction was diluted with water (4 mL) and extracted into EtOAc (3×5 mL). The combined EtOAc phases were washed with satd NaHCO₃ (2×10 mL), dried (Na₂SO₄) and filtered and the filtrate reduced in vacuo. Column chromatography (silica gel, 0-10% MeOH in DCM) afforded a 1:1 mixture of the title compounds as a vitreous yellow solid: LC/MS t_{R} 1.53 min; MS (ES+) m/z 612, 634; ¹H NMR δ_{H} (360 MHz, CDCl₃) 9.35 and 9.13 (1H, 2 br s), 8.42 and 8.41 (1H, 2 s), 7.80 (1H, d), 7.46 (2H, d), 7.23 (1H, app td), 7.07 (1H, m), 7.02 (2H, d), 6.98 (1H, obs m), 4.90 and 4.77 (2H, 2 s), 4.03-4.62 (1H, br m), 3.91 (3H, s), 3.78-3.92 (3H, obs m), 3.33 (2H, m), 3.10 (1H, br d), 2.60-2.91 (1H, m), 2.53 (3H, q), 2.39-2.59 (2H, obs m), 2.29 (0.5H, dd), 2.18 (0.5H, t), 1.43 and 1.31 (3H, 2 d).

### Example 168

### [(2R)-4-[(1R)-1-(2-Bromophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (203) and [(2R)-4-[(1S)-1-(2-Bromophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (204)

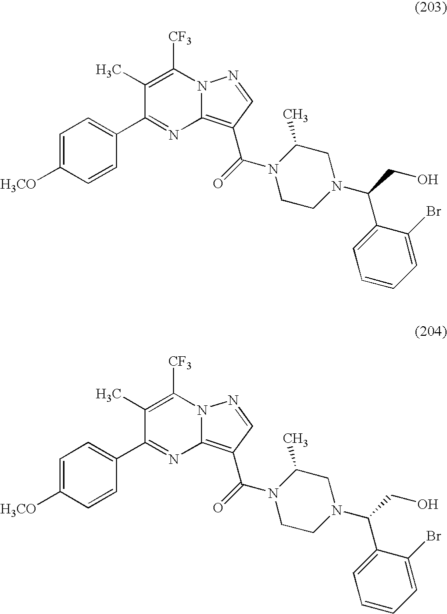

Piperazine 69 (50 mg, 0.12 mmol), glycolaldehyde dimer (8 mg, 0.06 mmol) and 2-bromophenylboronic acid (26 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.91 and 1.96 min; MS (ES+) m/z 632, 634; ¹H NMR δ_{H} (250 MHz, CDCl₃) 8.421 and 8.419 (1H, 2 s), 7.61 (1H, d), 7.48 (2H, d), 7.22-7.39 (2H, m), 7.16 (1H, t), 7.03 (2H, d), 4.24 (1H, m), 3.99-4.98 (1H, br m), 3.93 (3H, s), 3.60-3.98 (3H, m), 2.90-3.59 (2H, br m), 2.54 (3H, q), 2.38-2.88 (3H, m), 2.26 (0.5H, dd), 2.11 (0.5H, app td), 1.43 and 1.37 (3H, 2 d).

### Example 169

### [(2R)-4-[(1R)-1-(2-Ethoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (205) and [(2R)-4-[(1S)-1-(2-Ethoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (206)

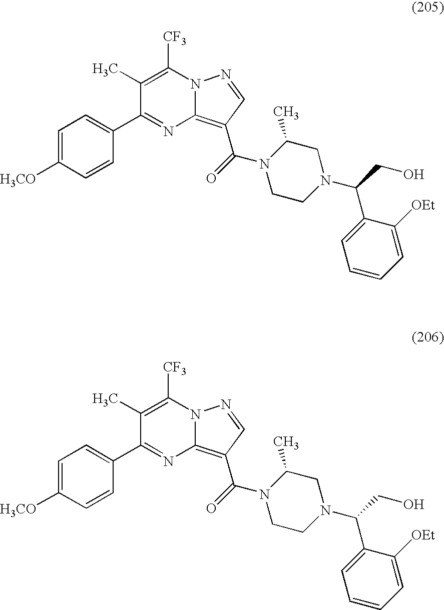

Piperazine 69 (50 mg, 0.12 mmol), glycolaldehyde dimer (8 mg, 0.06 mmol) and 2-ethoxyphenylboronic acid (21 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.67 min; MS (ES+) m/z 598; ¹H NMR δ_{H} (250 MHz, CDCl₃) 8.38 (1H, s), 7.47 (2H, d), 7.26 (1H, app td), 7.06 (1H, m), 7.01 (2H, d), 6.81-6.95 (2H, m), 4.38 (2H, m), 4.10-4.92 (1H, br m), 3.93 (3H, s), 3.87-4.09 (3H, obs m), 3.58 (1H, dd), 3.37 (1H, br s), 3.04 (1H, br d), 2.82 (1H, br d), 2.54 (3H, q), 2.30-2.75 (2H, m), 2.11 (0.5H, dd), 1.94 (0.5H, app td), 1.44 and 1.40 (3H, 2 d), 1.37 (3H, t).

### Example 170

### [(2R)-4-[(1R)-1-(2-Fluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (207) and [(2R)-4-[(1S)-1-(2-Fluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (208)

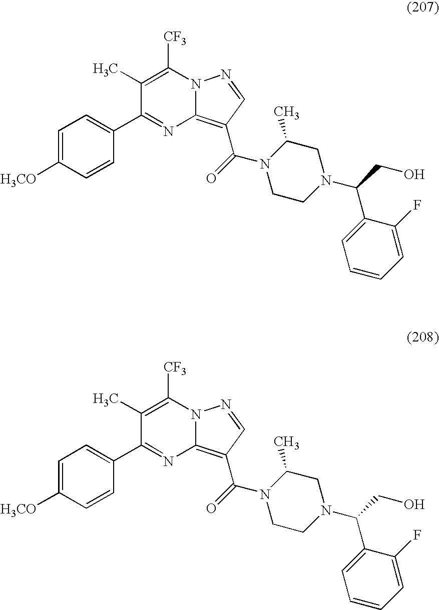

Piperazine 69 (500 mg, 1.15 mmol), glycolaldehyde dimer (76 mg, 0.63 mmol) and 2-fluorophenylboronic acid (178 mg, 1.27 mmol) were reacted in accordance with Method V to afford the title compounds after reverse phase preparative HPLC (5-95% MeCN in water) as vitreous yellow solids: for 207, >95% de; LC/MS t_{R} 1.70 min; MS (ES+) m/z 572; ¹H NMR δ_{H} (250 MHz, CDCl₃) 8.39 (1H, s), 7.45 (2H, d), 7.23-7.33 (1H, m), 6.98-7.18 (5H, m), 4.66 (1H, br s), 4.12-4.22 (1H, m), 3.92 (3H, s), 3.88-4.05 (1H, obs m), 3.57-3.69 (1H, m), 3.45 (1H, br s), 2.86-3.02 (2H, m), 2.68 (1H, br d), 2.52 (3H, q), 2.39-2.56 (1H, obs m), 2.10 (1H, dd), 1.38 (3H, d). For 208, >95% de; LC/MS t_{R} 1.75 min; MS (ES+) m/z 572; ¹H NMR δ_{H} (250 MHz, CDCl₃) 8.40 (1H, s), 7.46 (2H, d), 7.20-7.37 (1H, m), 6.88-7.21 (5H, m), 4.54 (1H, br s), 4.11 (2H, m), 3.94 (3H, s), 3.64 (1H, app td), 3.31 (1H, br s), 3.05 (1H, d), 2.70-2.91 (2H, m), 2.58-2.70 (1H, m), 2.53 (3H, q), 1.95 (1H, app td), 1.43 (3H, d).

### Example 171

### [(2R)-4-[(1R)-1-(3-Fluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (209) and [(2R)-4-[(1S)-1-(3-Fluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (210)

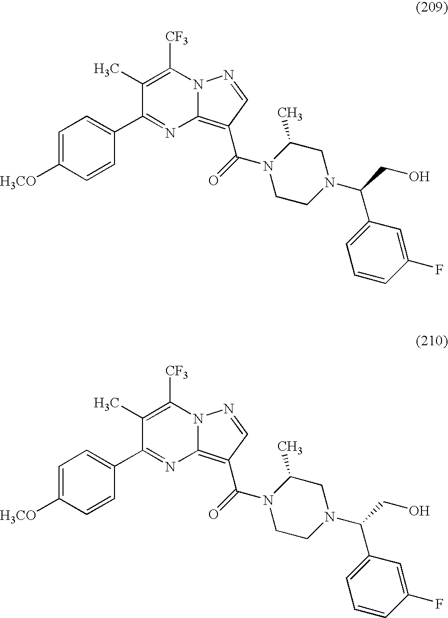

Piperazine 69 (50 mg, 0.12 mmol), glycolaldehyde dimer (8 mg, 0.06 mmol) and 3-fluorophenylboronic acid (18 mg, 0.13 mmol) were reacted in accordance with Method V to afford a 1:1 mixture of the title compounds after column chromatography (silica gel, 60-80% EtOAc in heptanes) as a vitreous yellow solid: LC/MS t_{R} 1.70 and 1.73 min; MS (ES+) m/z 572; ¹H NMR δ_{H} (250 MHz, CDCl₃) 8.404 and 8.400 (1H, 2 s), 7.46 (2H, d), 7.22-7.37 (1H, m), 6.83-7.07 (5H, m), 4.64 (1H, br m), 3.92 (3H, s), 3.84-3.97 (1H, obs m), 3.57-3.75 (2H, m), 3.34 (1H, br s), 2.53 (3H, q), 2.22-2.98 (5H, m), 2.12 (0.5H, dd), 1.96 (0.5H, app td), 1.43 and 1.37 (3H, 2 d).

### Example 172

### [(2R)-4-[(1R)-1-(2,5-Dimethoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (211) and [(2R)-4-[(1S)-1-(2,5-Dimethoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (212)

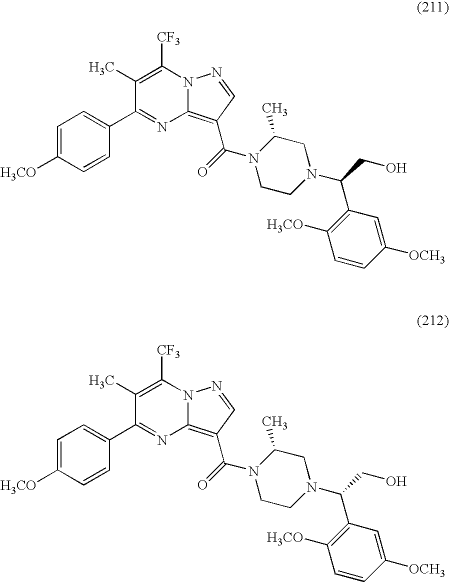

Piperazine 69 (50 mg, 0.12 mmol), glycolaldehyde dimer (8 mg, 0.06 mmol) and 2,5-dimethoxyphenylboronic acid (23 mg, 0.13 mmol) were reacted in accordance with Method V to afford a 1:1 mixture of the title compounds after column chromatography (silica gel, 40-50% EtOAc in heptanes) as a vitreous yellow solid: LC/MS t_{R} 1.63 min; MS (ES+) m/z 614; ¹H NMR δ_{H} (250 MHz, CDCl₃) 8.38 and 8.37 (1H, 2 s), 7.48 (2H, d), 7.01 (2H, d), 6.68-6.87 (3H, m), 4.59 (1H, br m), 4.22 (1H, m), 3.91 (3H, s), 3.90 (1H, obs m), 3.75 (3H, s), 3.71 (3H, s), 3.56-3.65 (1H, m), 3.38 (1H, br s), 2.53 (3H, q), 2.25-3.08 (5H, m), 2.12 (0.5H, dd), 1.94 (0.5H, app td), 1.43 and 1.39 (3H, 2 d).

### Example 173

### [(2R)-4-[(1R)-1-(2,3-Dimethoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (213) and [(2R)-4-[(1S)-1-(2,3-Dimethoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (214)

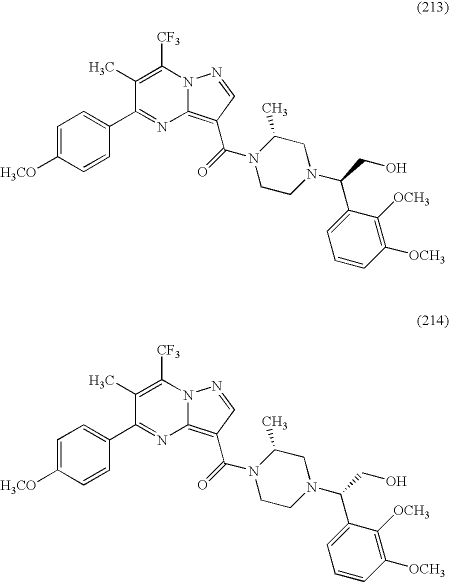

Piperazine 69 (50 mg, 0.12 mmol), glycolaldehyde dimer (8 mg, 0.06 mmol) and 2,3-dimethoxyphenylboronic acid (23 mg, 0.13 mmol) were reacted in accordance with Method V to afford a 1:1 mixture of the title compounds after column chromatography (silica gel, 40-50% EtOAc in heptanes) as a vitreous yellow solid: LC/MS t_{R} 1.62 min; MS (ES+) m/z 614; ¹H NMR δ_{H} (250 MHz, CDCl₃) 8.39 and 8.38 (1H, s), 7.48 (2H, d), 7.03 (2H, d), 7.01 (1H, obs m), 6.87 (1H, d), 6.74 (1H, t), 4.66 (1H, br m), 4.21 (1H, m), 3.90 (3H, s), 3.90 (1H, obs m), 3.84 (3H, s), 3.77 (3H, s), 3.62 (1H, dd), 2.60-3.48 (4H, m), 2.53 (3H, q), 2.41 (1H, app td), 2.15 (0.5H, dd), 1.99 (0.5H, app td), 1.42 and 1.37 (3H, 2 d).

### Example 174

### [(2R)-4-[(1R)-1-(2,4-Dimethoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (215) and [(2R)-4-[(1S)-1-(2,4-Dimethoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (216)

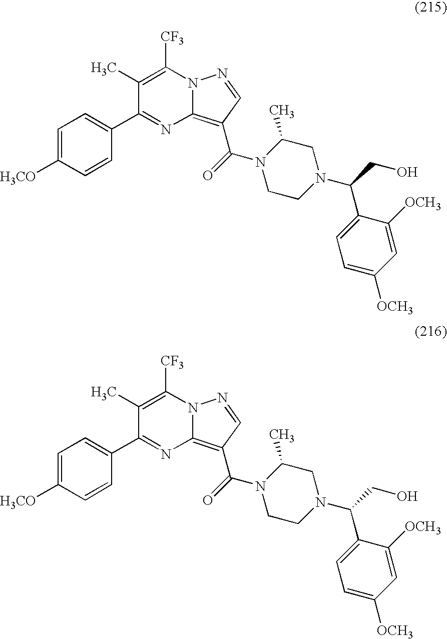

Piperazine 69 (50 mg, 0.12 mmol), glycolaldehyde dimer (8 mg, 0.06 mmol) and 2,4-dimethoxyphenylboronic acid (23 mg, 0.13 mmol) were reacted in accordance with Method V to afford a 1:1 mixture of the title compounds after column chromatography (silica gel, 40-50% EtOAc in heptanes) as a vitreous yellow solid: LC/MS t_{R} 1.60 min; MS (ES+) m/z 614; ¹H NMR δ_{H} (250 MHz, CDCl₃) 8.37 (1H, s), 7.47 (2H, d), 7.02 (2H, d), 6.97 (1H, obs d), 6.46 (1H, s), 6.43 (1H, d), 4.63 (1H, br s), 4.25 (1H, m), 3.92 (3H, s), 3.91 (1H, obs m), 3.81 (3H, s), 3.80 (1H, m), 3.72 (3H, s), 3.55 (1H, dd), 3.43 (1H, br s), 2.59-2.97 (2H, m), 2.53 (3H, q), 2.21-2.58 (2H, m), 2.10 (0.5H, dd), 1.94 (0.5H, app td), 1.43 and 1.39 (3H, 2 d).

### Example 175

### [(2R)-4-[(1R)-1-(5-Fluoro-2-methoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (217) and [(2R)-4-[(1S)-1-(5-Fluoro-2-methoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (218)

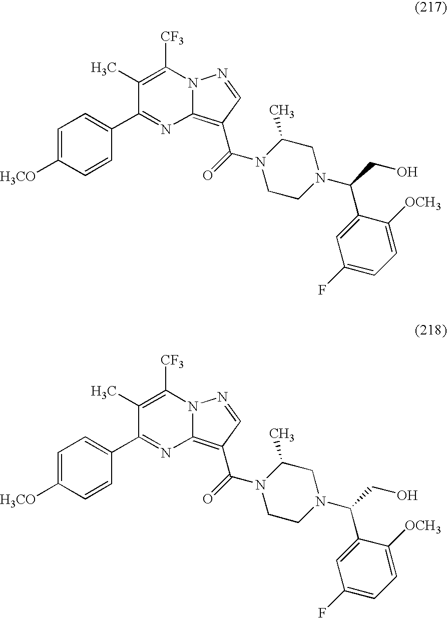

Piperazine 69 (50 mg, 0.12 mmol), glycolaldehyde dimer (8 mg, 0.06 mmol) and 5-fluoro-2-methoxyphenylboronic acid (22 mg, 0.13 mmol) were reacted in accordance with Method V to afford a 1:1 mixture of the title compounds after column chromatography (silica gel, 40-50% EtOAc in heptanes) as a vitreous yellow solid: LC/MS t_{R} 1.62 and 1.67 min; MS (ES+) m/z 602; ¹H NMR δ_{H} (250 MHz, CDCl₃) 8.40 and 8.39 (1H, 2 s), 7.48 (2H, d), 7.02 (2H, d), 6.72-7.06 (3H, m), 4.65 (1H, br s), 4.25 (1H, m), 3.92 (3H, s), 3.90 (2H, obs m), 3.74 (3H, s), 3.60 (1H, dd), 3.39 (1H, br s), 2.70-3.10 (2H, m), 2.54 (3H, q), 2.22-2.70 (2H, m), 2.14 (0.5H, dd), 1.98 (0.5H, app td), 1.44 and 1.39 (3H, 2 d).

### Example 176

### [(2R)-4-[(1R)-1-(2-Fluoro-6-methoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (219) and [(2R)-4-[(1S)-1-(2-Fluoro-6-methoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (220)

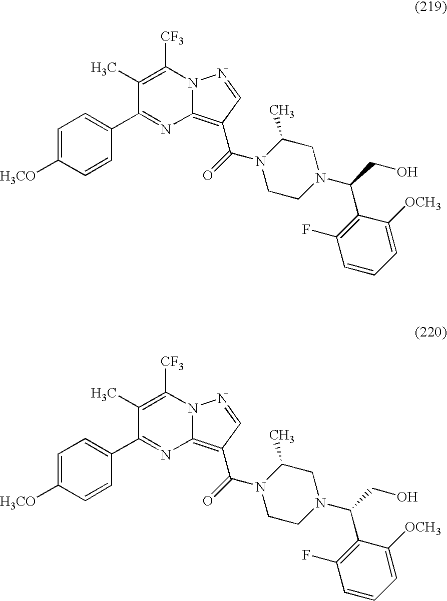

Piperazine 69 (50 mg, 0.12 mmol), glycolaldehyde dimer (8 mg, 0.06 mmol) and 2-fluoro-6-methoxyphenylboronic acid (22 mg, 0.13 mmol) were reacted in accordance with Method V to afford a 1:1 mixture of the title compounds after column chromatography (silica gel, 40-50% EtOAc in heptanes) as a vitreous yellow solid: LC/MS t_{R} 1.60 and 1.65 min; MS (ES+) m/z 602; ¹H NMR δ_{H} (250 MHz, CDCl₃) 8.38 (1H, s), 7.47 (2H, d), 7.24 (1H, m), 7.02 (2H, d), 6.59-6.74 (2H, m), 4.21-5.09 (3H, br m), 3.93 (3H, s), 3.74 (3H, s), 3.19-3.64 (2H, m), 2.64-3.16 (3H, m), 2.53 (3H, q), 2.29-2.59 (1H, m), 2.13 (0.5H, dd), 1.97 (0.5H, app td), 1.42 and 1.40 (3H, 2 d).

### Example 177

### [(2R)-4-[(1R)-1-(3,5-Difluoro-2-methoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (221) and [(2R)-4-[(is)-1-(3,5-Difluoro-2-methoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (222)

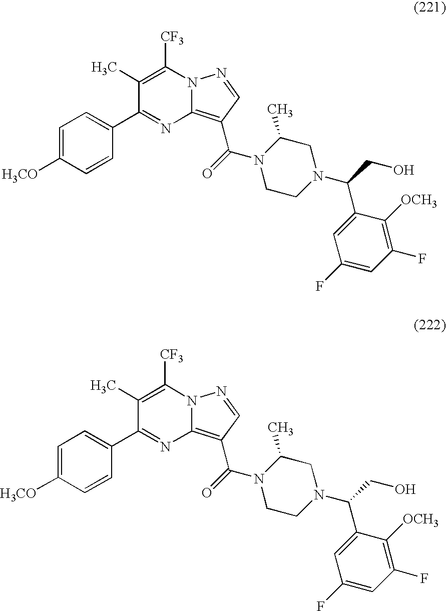

Piperazine 69 (50 mg, 0.12 mmol), glycolaldehyde dimer (8 mg, 0.06 mmol) and 3,5-difluoro-2-methoxyphenylboronic acid (24 mg, 0.13 mmol) were reacted in accordance with Method V to afford a 1:1 mixture of the title compounds after column chromatography (silica gel, 0-2% MeOH in DCM) as a vitreous yellow solid: LC/MS t_{R} 1.86 and 1.92 min; MS (ES+) m/z 620; ¹H NMR δ_{H} (360 MHz, CDCl₃) 8.42 and 8.41 (1H, 2 s), 7.47 (2H, d), 7.02 (2H, d), 6.82 (1H, m), 6.70 (1H, br s), 4.57 (1H, br m), 4.16 (1H, m), 3.92 (3H, s), 3.82 (3H, s), 3.78-4.02 (2H, obs m), 3.65 (1H, m), 3.36 (1H, br s), 2.53 (3H, q), 2.38-3.02 (4H, m), 2.17 (0.5H, dd), 2.00 (0.5H, app td), 1.42 and 1.37 (3H, 2 d).

### Example 178

### [(2R)-4-[(1S)-1-(2-Benzofuranyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (223) and [(2R)-4-[(1R)-1-(2-Benzofuranyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (224)

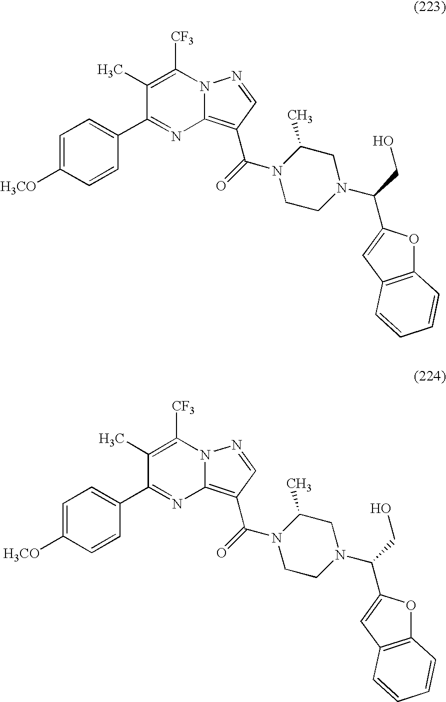

Piperazine 69 (50 mg, 0.12 mmol), glycolaldehyde dimer (8 mg, 0.06 mmol) and 2-benzofuranylboronic acid (21 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.88 and 1.98 min; MS (ES+) m/z 594; ¹H NMR δ_{H} (250 MHz, CDCl₃) 8.39 (1H, s), 7.53 (1H, dd), 7.47 (2H, d), 7.41 (1H, d), 7.28 (1H, app td), 7.23 (1H, app td), 6.95 (2H, br d), 6.50 (1H, s), 4.65 (1H, br s), 4.00 (2H, m), 3.90 (3H, s), 3.82 (1H, dd), 3.38 (1H, br s), 2.63-3.10 (3H, m), 2.53 (3H, q), 1.99-2.29 (2H, m), 1.43 and 1.40 (3H, 2 d).

### Example 179

### [(2R)-4-[(1R)-1-(4-Fluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (225) and [(2R)-4-[(1S)-1-(4-Fluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (226)

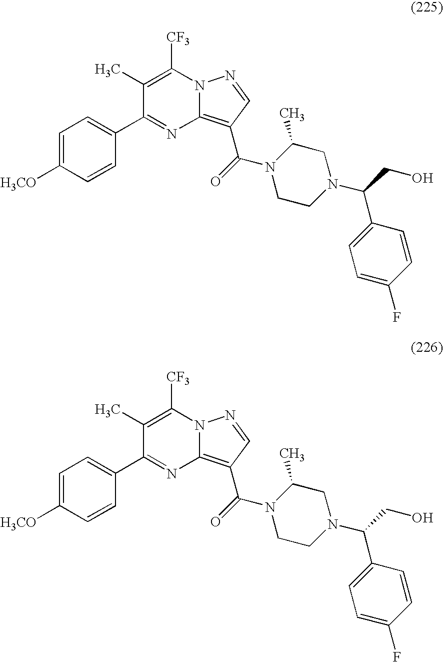

Piperazine 69 (50 mg, 0.12 mmol), glycolaldehyde dimer (8 mg, 0.06 mmol) and 4-fluorophenylboronic acid (18 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.64 and 1.66 min; MS (ES+) m/z 572; ¹H NMR δ_{H} (250 MHz, CDCl₃) 8.41 and 8.40 (1H, 2 s), 7.45 (2H, d), 7.10 (2H, m), 7.02 (2H, d), 7.00 (2H, obs m), 4.60 (1H, br m), 3.93 (3H, s), 3.92 (1H, obs m), 3.65 (2H, m), 3.36 (1H, br s), 2.66-3.09 (2H, br m), 2.53 (3H, q), 2.30-2.66 (2H, m), 2.08 (0.5H, dd), 1.92 (0.5H, app td), 1.43 and 1.37 (3H, 2 d).

### Example 180

### [(2R)-4-[(1R)-1-(3-Bromophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (227) and [(2R)-4-[(1S)-1-(3-Bromophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (228)

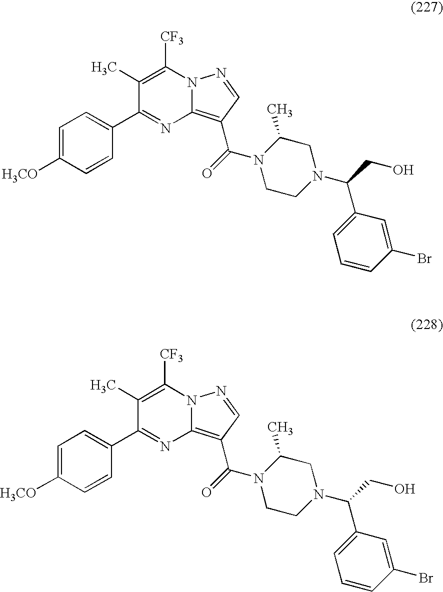

Piperazine 69 (200 mg, 0.46 mmol), glycolaldehyde dimer (30 mg, 0.26 mmol) and 3-bromophenylboronic acid (102 mg, 0.50 mmol) were reacted in accordance with Method V to afford a 3:2 mixture of the title compounds after reverse phase preparative HPLC (5-95% MeCN in water) as a vitreous yellow solid: LC/MS t_{R} 1.83 and 1.86 min; MS (ES+) m/z 632, 634; ¹H NMR δ_{H} (360 MHz, CDCl₃) 8.41 (1H, s), 7.47 (2H, d), 7.44 (1H, obs m), 7.33 (1H, d), 7.20 (1H, t), 7.09 (1H, dd), 7.03 (2H, d), 4.12-4.99 (1H, br s), 3.95 (3H, s), 3.93 (1H, obs m), 3.71 (1H, m), 3.59 (1H, m), 3.24-3.98 (2H, br m), 2.59-2.95 (3H, m), 2.53 (3H, q), 2.45 (1H, app td), 2.11 (0.5H, dd), 1.97 (0.5H, td), 1.44 and 1.38 (3H, 2 d).

### Example 181

### [(2R)-4-[(1R)-1-(4-Fluoro-2-methoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (229) and [(2R)-4-[(1S)-1-(4-Fluoro-2-methoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (230)

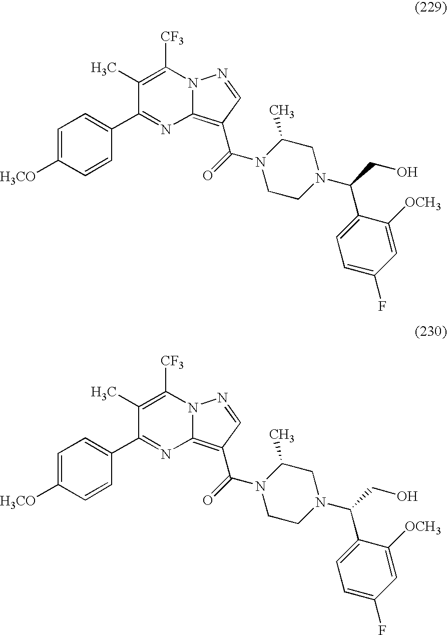

Piperazine 69 (50 mg, 0.12 mmol), glycolaldehyde dimer (8 mg, 0.06 mmol) and 4-fluoro-2-methoxyphenylboronic acid (22 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.63 min; MS (ES+) m/z 602; ¹H NMR δ_{H} (250 MHz, CDCl₃) 8.38 (1H, s), 7.46 (2H, d), 7.02 (1H, obs m), 7.01 (2H, d), 6.51-6.67 (2H, m), 4.24 (1H, m), 4.03-5.07 (1H, br m), 3.93 (3H, s), 3.90 (1H, obs m), 3.73 (3H, s), 3.55 (1H, m), 3.39 (1H, br s), 2.95 (1H, br s), 2.77 (1H, br s), 2.52 (3H, q), 2.23-2.68 (2H, m), 2.08 (0.5H, dd), 1.92 (0.5H, app td), 1.43 and 1.37 (3H, 2 d).

### Example 182

### [(2R)-4-[(1R)-1-(2-Fluoro-3-methoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (231) and [(2R)-4-[(1S)-1-(2-Fluoro-3-methoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (232)

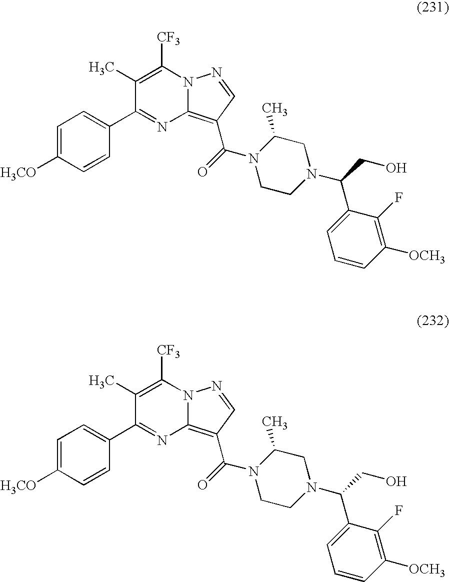

Piperazine 69 (200 mg, 0.46 mmol), glycolaldehyde dimer (30 mg, 0.25 mmol) and 2-fluoro-3-methoxyphenylboronic acid (86 mg, 0.51 mmol) were reacted in accordance with Method V to afford the title compounds after reverse phase preparative HPLC (5-95% MeCN in water) as vitreous yellow solids: for 231, >95% de; LC/MS t_{R} 1.71 min; MS (ES+) m/z 602; ¹H NMR δ_{H} (250 MHz, CDCl₃) 8.41 (1H, s), 7.48 (2H, d), 6.85-7.10 (4H, m), 6.70 (1H, t), 4.74 (1H, br m), 4.21 (1H, m), 3.94 (3H, s), 3.91-4.07 (1H, obs m), 3.89 (3H, s), 3.75-3.90 (1H, obs m), 3.64 (1H, dd), 3.25-3.75 (2H, br m), 2.95 (1H, br d), 2.70 (1H, br d), 2.55 (3H, q), 2.51 (1H, obs m), 2.13 (1H, dd), 1.40 (3H, d). For 232, >95% de; LC/MS t_{R} 1.77 min; MS (ES+) m/z 602; ¹H NMR δ_{H} (250 MHz, CDCl₃) 8.40 (1H, s), 7.48 (2H, d), 6.85-7.10 (4H, m), 6.70 (1H, t), 4.67 (1H, br m), 3.93 (3H, s), 3.89 (3H, s), 3.78-4.20 (3H, m), 3.64 (1H, app td), 3.28 (1H, br s), 3.05 (1H, d), 2.74-2.82 (2H, m), 2.82 (1H, br d), 2.54 (3H, q), 1.96 (1H, app td), 1.42 (3H, d).

### Example 183

### [(2R)-4-[(1R)-1-(3-Fluoro-5-methoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (233) and [(2R)-4-[(1S)-1-(3-Fluoro-5-methoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (234)

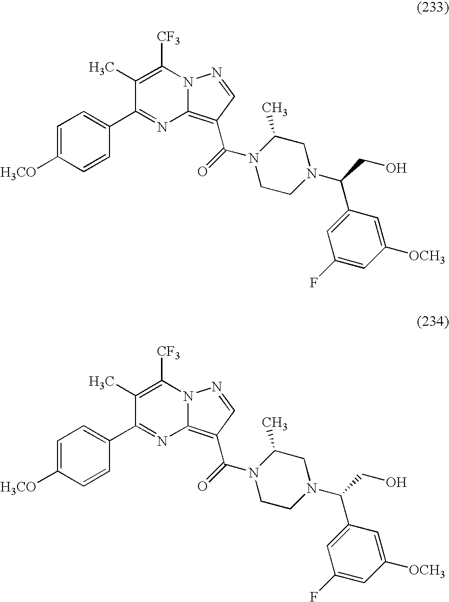

Piperazine 69 (50 mg, 0.12 mmol), glycolaldehyde dimer (8 mg, 0.06 mmol) and 3-fluoro-5-methoxyphenylboronic acid (22 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.76 and 178 min; MS (ES+) m/z 602; ¹H NMR δ_{H} (250 MHz, CDCl₃) 8.42 and 8.41 (1H, 2 s), 7.47 (2H, d), 7.03 (2H, d), 6.44-6.62 (3H, m), 4.63 (1H, br m), 3.93 (3H, s), 3.90 (1H, obs m), 3.79 (3H, s), 3.48-3.81 (3H, m), 3.31 (1H, br s), 2.93 (1H, br s), 2.77 (1H, br d), 2.67 (1H, br d), 2.54 (3H, q), 2.51 (1H, obs m), 2.20 (0.5H, dd), 2.02 (0.5H, app td), 1.43 and 1.38 (3H, 2 d).

### Example 184

### [(2R)-4-[(1R)-2-Hydroxy-1-(4-methoxyphenyl)ethyl]-2-methyl-1-piperazinyl][5-(4-methoxyphenyl)-6-methyl-7-(trifluoromethyl)pyrazolo[1,5-a]pyrimidin-3-yl]methanone (235) and [(2R)-4-[(1S)-2-Hydroxy-1-(4-methoxyphenyl)ethyl]-2-methyl-1-piperazinyl][5-(4-methoxyphenyl)-6-methyl-7-(trifluoromethyl)pyrazolo[1,5-a]pyrimidin-3-yl]methanone (236)

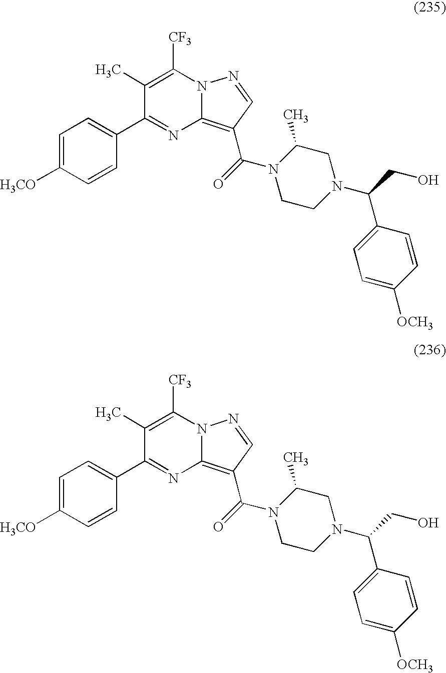

Piperazine 69 (50 mg, 0.12 mmol), glycolaldehyde dimer (8 mg, 0.06 mmol) and 4-methoxyphenylboronic acid (19 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.59 min; MS (ES+) m/z 584; ¹H NMR δ_{H} (250 MHz, CDCl₃) 8.40 and 8.39 (1H, 2 s), 7.46 (2H, d), 7.06 (2H, d), 7.03 (2H, d), 6.85 (2H, d), 4.62 (1H, br s), 3.93 (3H, s), 3.91 (1H, obs m), 3.81 (3H, s), 3.64 (2H, m), 3.40 (1H, br s), 2.58-3.16 (3H, m), 2.53 (3H, q), 2.24-2.57 (1H, obs m), 2.08 (0.5H, dd), 1.93 (0.5H, app td), 1.43 and 1.38 (3H, 2 d).

### Example 185

### 1-[3-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]ethanone (237) and 1-[3-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]ethanone (238)

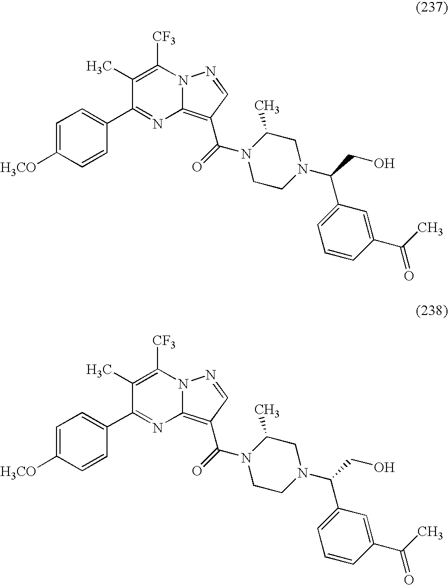

Piperazine 69 (50 mg, 0.12 mmol), glycolaldehyde dimer (8 mg, 0.06 mmol) and 3-acetylphenylboronic acid (21 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.66 min; MS (ES+) m/z 596, 618; ¹H NMR δ_{H} (250 MHz, CDCl₃) 8.39 and 8.38 (1H, 2 s), 7.90 (1H, d), 7.78 (1H, m), 7.46 (2H, d), 7.34-7.45 (2H, m), 7.01 (2H, d), 4.63 (1H, br s), 3.98 (1H, m), 3.93 (3H, s), 3.72 (2H, m), 3.42 (1H, br s), 2.64-3.09 (2H, m), 2.61 (3H, s), 2.53 (3H, q), 2.19-2.57 (2H, obs m), 2.08 (0.5H, dd), 1.93 (0.5H, app td), 1.45 and 1.40 (3H, 2 d).

### Example 186

### 4-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]benzonitrile (239) and 4-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]benzonitrile (240)

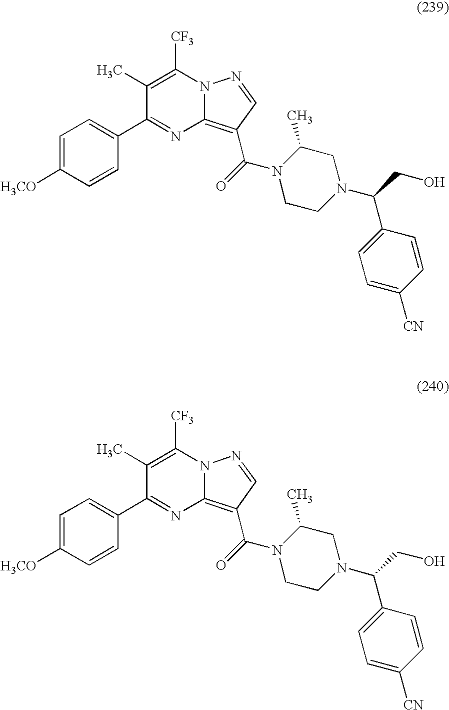

Piperazine 69 (50 mg, 0.12 mmol), glycolaldehyde dimer (8 mg, 0.06 mmol) and 4-cyanophenylboronic acid (19 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.76 and 1.78 min; MS (ES+) m/z 579; ¹H NMR δ_{H} (250 MHz, CDCl₃) 8.42 (1H, s), 7.62 (2H, d), 7.45 (2H, d), 7.30 (2H, d), 7.04 (2H, d), 4.63 (1H, br m), 3.95 (3H, s), 3.93 (1H, obs m), 3.76 (1H, m), 3.63 (1H, br s), 3.36 (1H, br s), 2.53 (3H, q), 2.18-3.18 (4H, m), 2.12 (0.5H, dd), 1.98 (0.5H, app td), 1.44 and 1.38 (3H, 2 d).

### Example 187

### 3-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]benzonitrile (241) and 3-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]benzonitrile (242)

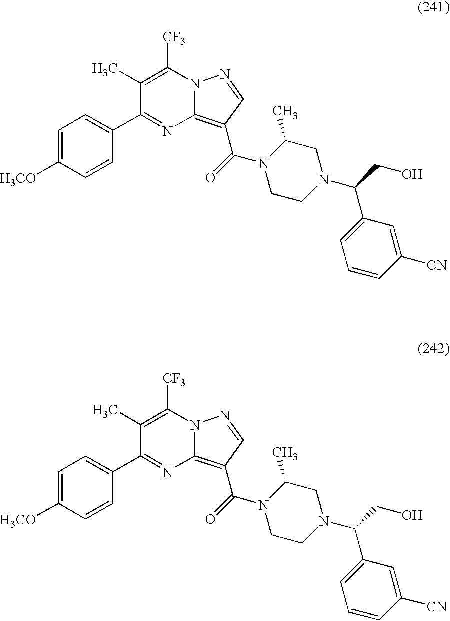

Piperazine 69 (50 mg, 0.12 mmol), glycolaldehyde dimer (8 mg, 0.06 mmol) and 3-cyanophenylboronic acid (19 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.78 and 1.84 min; MS (ES+) m/z 579; ¹H NMR δ_{H} (360 MHz, CDCl₃) 8.401 and 8.397 (1H, 2 s), 7.61 (1H, m), 7.41-7.53 (5H, m), 7.04 (2H, d), 4.06-4.90 (1H, br m), 3.93 (3H, s), 3.91 (1H, obs m), 3.76 (1H, m), 3.59 (1H, m), 3.43 (1H, br s), 2.62-3.10 (2H, m), 2.53 (3H, q), 2.37-2.62 (3H, m), 2.10 (0.5H, dd), 1.96 (0.5H, app td), 1.44 and 1.37 (3H, 2 d).

### Example 188

### 2-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]benzonitrile (243) and 2-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]benzonitrile (244)

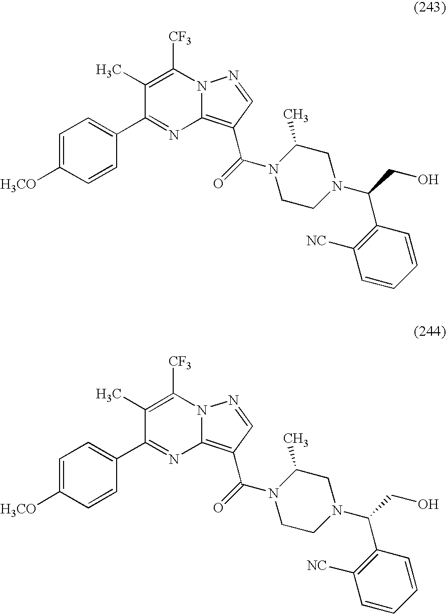

Piperazine 69 (50 mg, 0.12 mmol), glycolaldehyde dimer (8 mg, 0.06 mmol) and 2-cyanophenylboronic acid (19 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 2.02 and 2.05 min; MS (ES+) m/z 579; ¹H NMR δ_{H} (360 MHz, CDCl₃) 8.41 (1H, s), 7.69 (1H, d), 7.57 (1H, t), 7.38-7.51 (4H, m), 7.04 (2H, d), 4.20-4.90 (1H, br s), 4.10 (1H, m), 3.94 (1H, obs m), 3.94 (3H, s), 3.79 (1H, br s), 2.70-3.66 (3H, m), 2.53 (3H, q), 2.39-2.69 (3H, m), 2.24 (0.5H, d), 2.11 (0.5H, t), 1.42 and 1.37 (3H, 2 d).

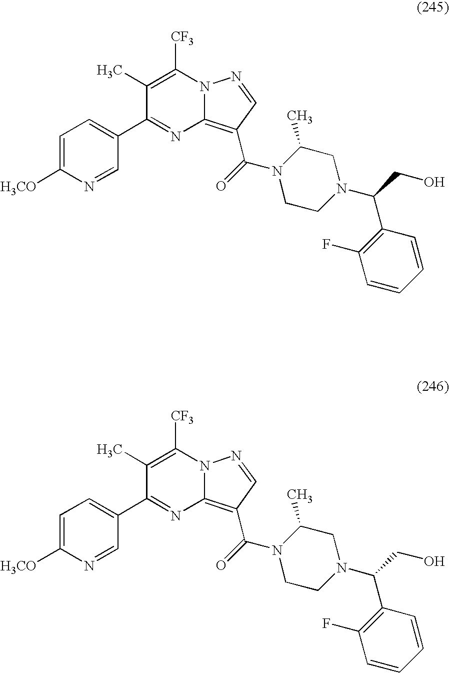

### Example 189

### (3R)-4-[[5-(6-Methoxy-3-pyridinyl)-6-methyl-7-(trifluoromethyl)pyrazolo[1,5-a]pyrimidin-3-yl]carbonyl]-3-methyl-1-piperazinecarboxylic Acid, 1,1-Dimethylethyl Ester (247)

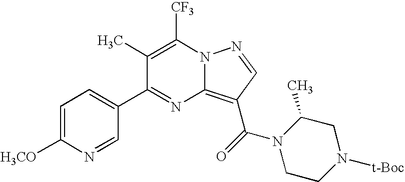

Carboxylic acid 127 (100 mg, 0.28 mmol) was treated with HATU (130 mg, 0.34 mmol), DIPEA (69 μL, 0.40 mmol, d 0.742) and piperazine 67 (63 mg, 0.31 mmol) in accordance with Method G to afford the title compound as a yellow powder: LC/MS t_{R} 2.20 min; MS (ES+) m/z 535, 557.

### Example 190

### [5-(6-Methoxy-3-pyridinyl)-6-methyl-7-(trifluoromethyl)pyrazolo[1,5-a]pyrimidin-3-yl][(2R)-2-methyl-1-piperazinyl]methanone (248)

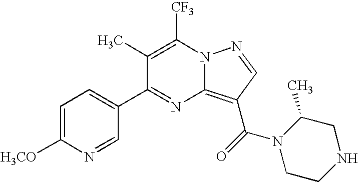

tert-Butyl carbamate 247 (100 mg, 0.19 mmol) was treated with TFA in accordance with Method O to afford the title compound as a pale yellow vitreous solid: LC/MS t_{R} 1.26 min; MS (ES+) m/z 435.

### Example 191

### [(2R)-4-[(1R)-1-(2-Fluorophenyl)-2-hydroxyethyl]-2-methyl-1-piperazinyl][5-(6-methoxy-3-pyridinyl)-6-methyl-7-(trifluoromethyl)pyrazolo[1,5-a]pyrimidin-3-yl]methanone (245) and [(2R)-4-[(1S)-1-(2-Fluorophenyl)-2-hydroxyethyl]-2-methyl-1-piperazinyl][5-(6-methoxy-3-pyridinyl)-6-methyl-7-(trifluoromethyl)pyrazolo[1,5-a]pyrimidin-3-yl]methanone (246)

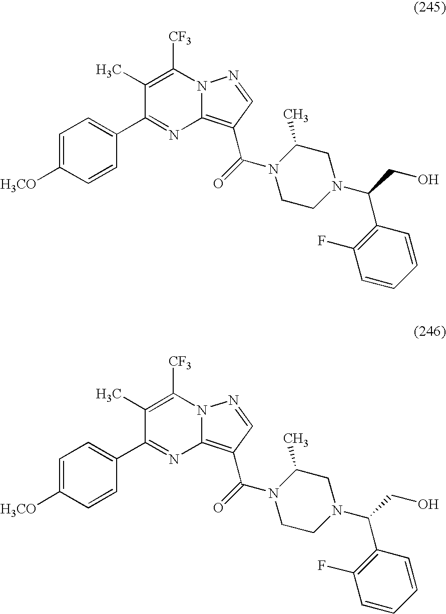

Piperazine 248 (40 mg, 0.09 mmol), glycolaldehyde dimer (6.1 mg, 0.05 mmol) and 2-fluorophenylboronic acid (14.2 mg, 0.10 mmol) were reacted in accordance with Method V to afford a 1:1 mixture of the title compounds after reverse phase preparative HPLC (5-95% MeCN in water) as a vitreous yellow solid: LC/MS t_{R} 1.58 and 1.63 min; MS (ES+) m/z 573; ¹H NMR δ_{H} (360 MHz, CDCl₃) 8.416 and 8.413 (1H, 2 s), 8.36 (1H, d), 7.75 (1H, dd), 7.26-7.33 (1H, m), 7.04-7.18 (3H, m), 6.89 (1H, d), 4.41 (1H, br s), 4.07 (3H, s), 3.95-4.20 (3H, m), 3.63 (1H, m), 3.22-3.53 (1H, br m), 2.58-3.12 (3H, m), 2.56 (3H, q), 2.44 (1H, t), 2.09 (0.5H, dd), 1.93 (0.5H, app td), 1.43 and 1.40 (3H, 2 d).

### Example 192

### (αR,3R)-4-[[5-(4-Methoxyphenyl)-6-methyl-7-(trifluoromethyl)pyrazolo[1,5-a]pyrimidin-3-yl]carbonyl]-3-methyl-α-phenyl-1-piperazineacetic Acid (249) and (αS,3R)-4-[[5-(4-Methoxyphenyl)-6-methyl-7-(trifluoromethyl)pyrazolo[1,5-a]pyrimidin-3-yl]carbonyl]-3-methyl-α-phenyl-1-piperazineacetic Acid (250)

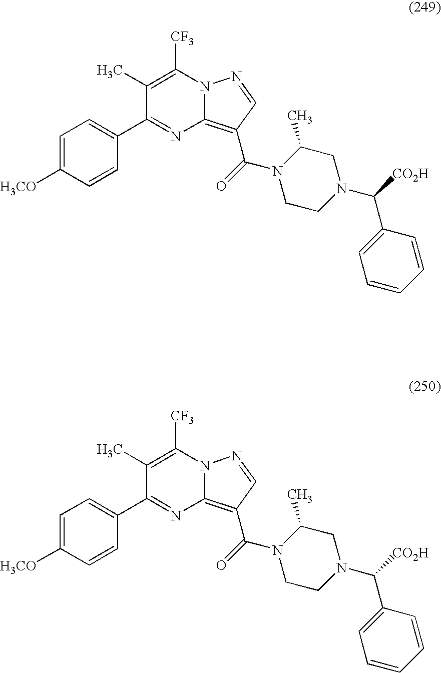

Piperazine 69 (300 mg, 0.69 mmol), glyoxylic acid monohydrate (70 mg, 0.76 mmol) and phenylboronic acid (94 mg, 0.76 mmol) were reacted in accordance with Method V to afford a 1:1 mixture of the title compounds after column chromatography (silica gel, 0-5% MeOH in DCM) as a vitreous yellow solid: LC/MS t_{R} 1.77 min; MS (ES+) m/z 568; ¹H NMR δ_{H} (250 MHz, CDCl₃) 8.44 and 8.43 (1H, 2 s), 7.46 (2H, d), 7.29 (5H, m), 7.04 (2H, d), 5.29 (1H, br s), 4.66 (1H, br s), 4.13 (1H, br d), 3.92 (3H, s), 3.41 (1H, br s), 2.87-3.26 (1H, m), 2.67 (1H, br s), 2.54 (3H, q), 2.48 (1H, obs m), 2.23 (1H, br d), 1.38 (3H, d).

### Example 193

### (αR,3R)-4-[[5-(4-Methoxyphenyl)-6-methyl-7-(trifluoromethyl)pyrazolo[1,5-a]pyrimidin-3-yl]carbonyl]-3-methyl-α-phenyl-1-piperazineacetamide (251) and (αS,3R)-4-[[5-(4-Methoxyphenyl)-6-methyl-7-(trifluoromethyl)pyrazolo[1,5-a]pyrimidin-3-yl]carbonyl]-3-methyl-α-phenyl-1-piperazineacetamide (252)

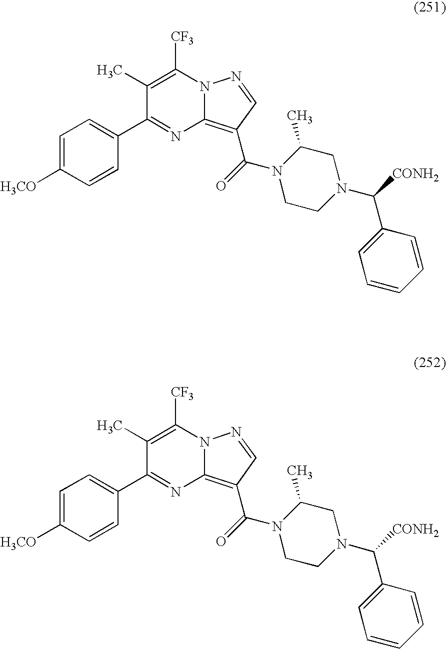

The 1:1 mixture of carboxylic acids 249 and 250 (30 mg, 0.053 mmol) was treated with HATU (24 mg, 0.063 mmol), DIPEA (27.5 μL, 0.16 mmol, d 0.742) and ammonium chloride (5.7 mg, 0.106 mmol) employing the procedure of Method G. Dilution of the reaction mixture with water (5 mL) and isolation of the resultant precipitate via vacuum filtration afforded a 1:1 mixture of the title compounds as an orange powder: LC/MS t_{R} 1.82 and 1.91 min; MS (ES+) m/z 567; ¹H NMR δ_{H} (250 MHz, CDCl₃) 8.433 and 8.427 (1H, 2 s), 7.46 (2H, d), 7.33 (5H, s), 7.03 (2H, d), 6.90 (1H, m), 5.65 (1H, br s), 4.07-5.02 (1H, br s), 3.94 (3H, s), 3.60-4.07 (2H, m), 3.37 (1H, br d), 3.00 (1H, dd), 2.53 (3H, q), 2.24-2.70 (2H, obs m), 2.12 (0.5H, dd), 2.00 (0.5H, app td), 1.44 and 1.38 (3H, 2 d).

### Example 194

### (αR,3R)-4-[[5-(4-Methoxyphenyl)-6-methyl-7-(trifluoromethyl)pyrazolo[1,5-a]pyrimidin-3-yl]carbonyl]-N,3-dimethyl-α-phenyl-1-piperazineacetamide (253) and (αS,3R)-4-[[5-(4-Methoxyphenyl)-6-methyl-7-(trifluoromethyl)pyrazolo[1,5-a]pyrimidin-3-yl]carbonyl]-N,3-dimethyl-α-phenyl-1-piperazineacetamide (254)

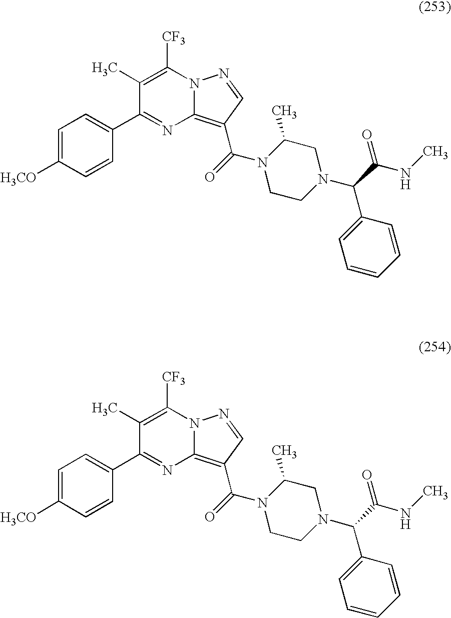

The 1:1 mixture of carboxylic acids 249 and 250 (30 mg, 0.053 mmol) was treated with HATU (24 mg, 0.063 mmol), DIPEA (27.5 μL, 0.16 mmol, d 0.742) and methylamine hydrochloride (5.4 mg, 0.079 mmol) employing the procedure of Method G. Dilution of the reaction mixture with water (5 mL) and isolation of the resultant precipitate via vacuum filtration afforded a 1:1 mixture of the title compounds as an orange powder: LC/MS t_{R} 1.89 and 1.99 min; MS (ES+) m/z 581; ¹H NMR δ_{H} (250 MHz, CDCl₃) 8.432 and 8.424 (1H, 2 s), 7.46 (2H, d), 7.31 (5H, s), 7.03 (2H, d), 6.92 (1H, m), 4.00-4.79 (1H, br s), 3.94 (3H, s), 3.56-3.99 (2H, m), 3.33 (1H, br d), 2.90 (1H, obs m), 2.87 (3H, d), 2.53 (3H, q), 2.31-2.57 (2H, obs m), 2.09 (0.5H, dd), 1.99 (0.5H, t), 1.45 and 1.38 (3H, 2 d).

### Example 195

### (αR,3R)-4-[[5-(4-Methoxyphenyl)-6-methyl-7-(trifluoromethyl)pyrazolo[1,5-a]pyrimidin-3-yl]carbonyl]-N,N,3-trimethyl-α-phenyl-1-piperazineacetamide (255) and (αS,3R)-4-[[5-(4-Methoxyphenyl)-6-methyl-7-(trifluoromethyl)pyrazolo[1,5-a]pyrimidin-3-yl]carbonyl]-N,N,3-trimethyl-α-phenyl-1-piperazineacetamide (256)

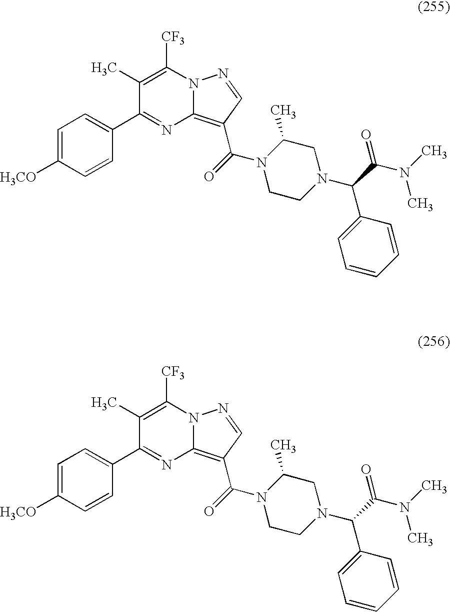

The 1:1 mixture of carboxylic acids 249 and 250 (30 mg, 0.053 mmol) was treated with HATU (24 mg, 0.063 mmol), DIPEA (27.5 μL, 0.16 mmol, d 0.742) and dimethylamine hydrochloride (6.5 mg, 0.079 mmol) employing the procedure of Method G. Dilution of the reaction mixture with water (5 mL) and isolation of the resultant precipitate via vacuum filtration afforded a 1:1 mixture of the title compounds as an orange powder: LC/MS t_{R} 1.63 and 1.71 min; MS (ES+) m/z 595; ¹H NMR δ_{H} (250 MHz, CDCl₃) 8.402 and 8.396 (1H, 2 s), 7.50 (2H, d), 7.34 (5H, m), 7.03 (2H, d), 4.11-4.91 (1H, br s), 4.32 (1H, br d), 3.92 (3H, s), 3.09-4.09 (2H, br m), 2.99 and 2.96 (3H, s), 2.94 and 2.93 (3H, s), 2.59-2.86 (2H, m), 2.53 (3H, q), 2.32-2.58 (1H, obs m), 2.22 (1H, br d), 1.41 and 1.39 (3H, 2 d).

### Example 196

### (αR,3R)-4-[[5-(4-Methoxyphenyl)-6-methyl-7-(trifluoromethyl)pyrazolo[1,5-a]pyrimidin-3-yl]carbonyl]-3-methyl-α-phenyl-1-piperazineacetic Acid, Methyl Ester (257) and (αS,3R)-4-[[5-(4-Methoxyphenyl)-6-methyl-7-(trifluoromethyl)pyrazolo[1,5-a]pyrimidin-3-yl]carbonyl]-3-methyl-α-phenyl-1-piperazineacetic Acid, Methyl Ester (258)

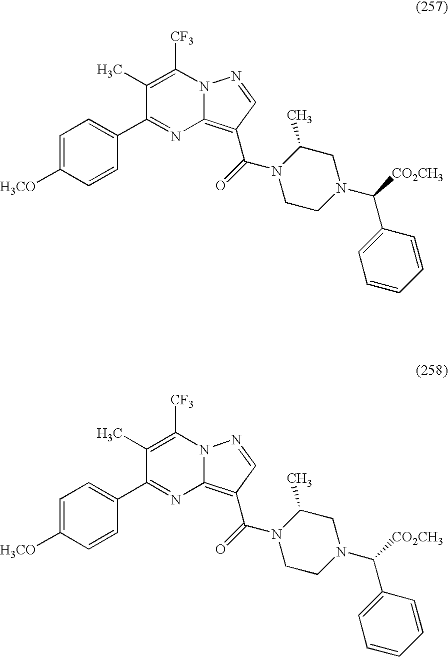

The 1:1 mixture of carboxylic acids 249 and 250 (99 mg, 0.17 mmol) in a 1:1 mixture of DCM and MeOH (3.5 mL) was treated with a solution of (trimethylsilyl)diazomethane (114 μL, 0.23 mmol, 2 M in hexanes) and stirred at rt for 1 h. Reaction progress was monitored by LC/MS. A further aliquot of (trimethylsilyl)diazomethane solution (28 μL, 0.056 mmol, 2 M in hexanes) was added and the reaction stirred a further hour at rt. AcOH (100 μL) was added and the reaction stirred 15 min further before diluting with DCM (15 mL) and washing with satd NaHCO₃ (2×10 mL) and brine (10 mL). The organic phase was dried (Na₂SO₄), filtered and reduced in vacuo and the residue purified by column chromatography (silica gel, 40% EtOAc in heptanes) to afford a 1:1 mixture of the title compounds as a vitreous yellow solid: LC/MS t_{R} 2.32 min; MS (ES+) m/z 582, 604; ¹H NMR δ_{H} (360 MHz, CDCl₃) 8.42 (1H, s), 7.50 (2H, d), 7.42 (2H, m), 7.33 (3H, m), 7.02 (2H, d), 4.17-5.02 (1H, br s), 4.02 (1H, d), 3.93 (3H, s), 3.89 (1H, d), 3.67 (3H, s), 3.46 (1H, br s), 2.59-3.02 (1H, br d), 2.54 (3H, q), 2.26-2.58 (2H, m), 2.21 (0.5H, dd), 2.15 (0.5H, app td), 1.43 and 1.37 (3H, 2 d).

### Example 197

### [(2R)-4-[(1R)-1-(2,3-Difluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (259) and [(2R)-4-[(1S)-1-(2,3-Difluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (260)

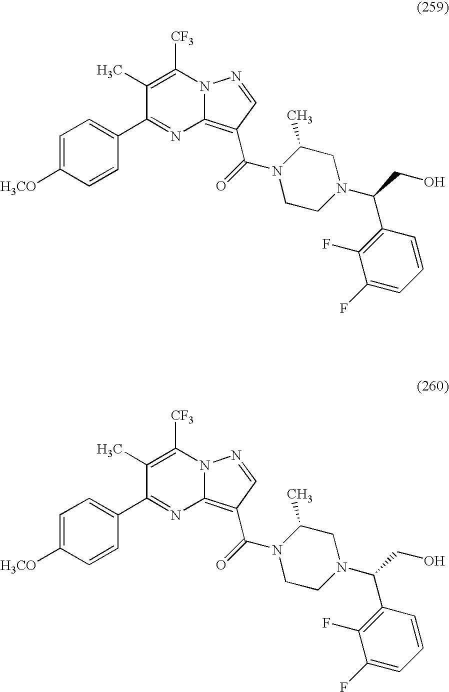

Piperazine 69 (50 mg, 0.12 mmol), glycolaldehyde dimer (8 mg, 0.06 mmol) and 2,3-difluorophenylboronic acid (20 mg, 0.13 mmol) were reacted in accordance with Method V to afford a 1:1 mixture of the title compounds. Purification by reverse phase preparative HPLC (5-95% MeCN in water) afforded a 4:1 mixture of the title compounds as a vitreous yellow solid: LC/MS t_{R} 1.89 and 1.95 min; MS (ES+) m/z 590; ¹H NMR δ_{H} (250 MHz, CDCl₃) 8.42 (1H, s), 7.46 (2H, d), 7.12 (2H, obs m), 7.04 (2H, d), 6.92 (1H, m), 4.38-4.99 (1H, br s), 3.94 (3H, s), 3.77-4.32 (3H, m), 3.67 (1H, m), 3.48 (1H, br s), 2.54 (3H, q), 2.43-3.05 (4H, m), 2.13 (0.5H, d), 1.96 (0.5H, t), 1.39 (3H, d).

### Example 198

### [(2R)-4-[(1R)-1-(2,4-Difluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (261) and [(2R)-4-[(1S)-1-(2,4-Difluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (262)

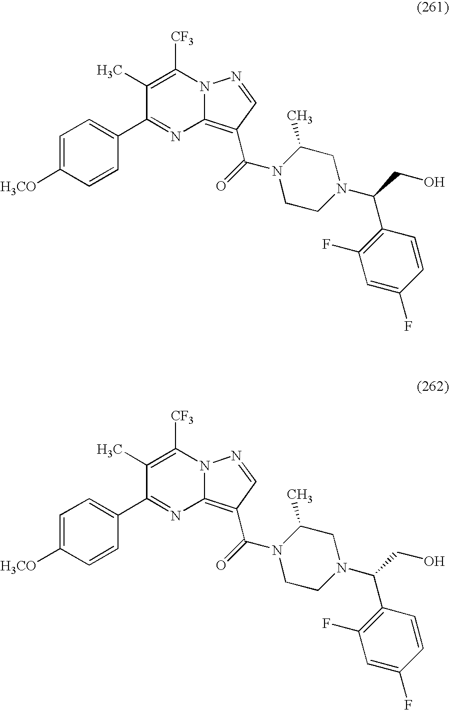

Piperazine 69 (50 mg, 0.12 mmol), glycolaldehyde dimer (8 mg, 0.06 mmol) and 2,4-difluorophenylboronic acid (20 mg, 0.13 mmol) were reacted in accordance with Method V to afford a 3:2 mixture of the title compounds after reverse phase preparative HPLC (5-95% MeCN in water) as a vitreous yellow solid: LC/MS t_{R} 1.80 and 1.86 min; MS (ES+) m/z 590; ¹H NMR δ_{H} (250 MHz, CDCl₃) 8.41 (1H, s), 7.45 (2H, d), 7.11 (1H, m), 7.03 (2H, d), 6.83 (2H, m), 4.24-4.94 (1H, br s), 4.05 (1H, m), 3.94 (1H, obs m), 3.94 (3H, s), 3.62 (1H, br d), 3.41 (1H, br s), 2.91 (1H, br s), 2.77 (1H, br d), 2.63 (1H, t), 2.53 (3H, q), 2.44 (1H, t), 2.09 (0.5H, dd), 1.93 (0.5H, app td), 1.42 and 1.38 (3H, 2 d).

### Example 199

### [(2R)-4-[(1R)-1-(2,5-Difluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (263) and [(2R)-4-[(1S)-1-(2,5-Difluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (264)

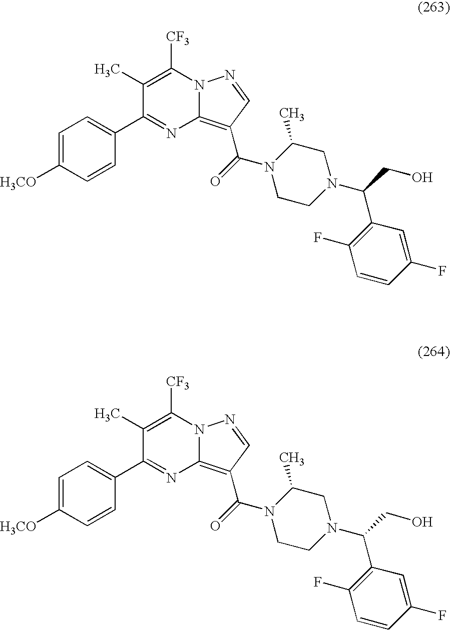

Piperazine 69 (50 mg, 0.12 mmol), glycolaldehyde dimer (8 mg, 0.06 mmol) and 2,5-difluorophenylboronic acid (20 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.91 and 1.96 min; MS (ES+) m/z 590; ¹H NMR δ_{H} (250 MHz, CDCl₃) 8.42 and 8.41 (1H, 2 s), 7.46 (2H, d), 7.03 (2H, d), 6.95-7.10 (2H, obs m), 6.87 (1H, m), 4.34-5.08 (1H, br s), 4.01-4.16 (1H, m), 3.93 (3H, s), 3.82-4.00 (1H, obs m), 3.67 (1H, dd), 3.40 (1H, br m), 2.59-3.02 (3H, m), 2.53 (3H, q), 2.40-2.58 (1H, obs m), 2.16 (0.5H, dd), 1.99 (0.5H, app td), 1.43 and 1.39 (3H, 2 d).

### Example 200

### [(2R)-4-[(1R)-1-(2,6-Difluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (265) and [(2R)-4-[(1S)-1-(2,6-Difluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (266)

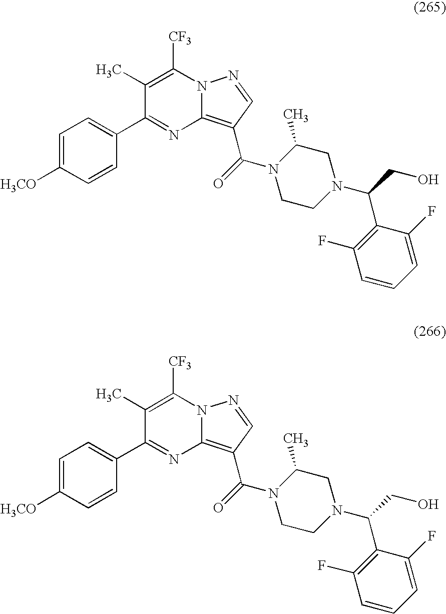

Piperazine 69 (50 mg, 0.12 mmol), glycolaldehyde dimer (8 mg, 0.06 mmol) and 2,6-difluorophenylboronic acid (20 mg, 0.13 mmol) were reacted in accordance with Method V to afford a 3:2 mixture of the title compounds after reverse phase preparative HPLC (5-95% MeCN in water) followed by column chromatography (silica gel, 60% EtOAc in heptanes) as a vitreous yellow solid: LC/MS t_{R} 1.79 and 1.85 min; MS (ES+) m/z 590; ¹H NMR δ_{H} (360 MHz, CDCl₃) 8.42 (1H, br s), 7.45 (2H, d), 7.28 (1H, obs br s), 7.04 (2H, d), 6.89 (2H, br s), 3.94 (3H, s), 3.83-4.99 (3H, m), 3.11-3.79 (2H, m), 2.58-3.10 (2H, m), 2.53 (3H, q), 2.30-2.56 (2H, obs m), 1.85-2.28 (1H, m), 1.43 (3H, m).

### Example 201

### [(2R)-4-[(1R)-1-(3,5-Difluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (267) and [(2R)-4-[(1S)-1-(3,5-Difluorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (268)

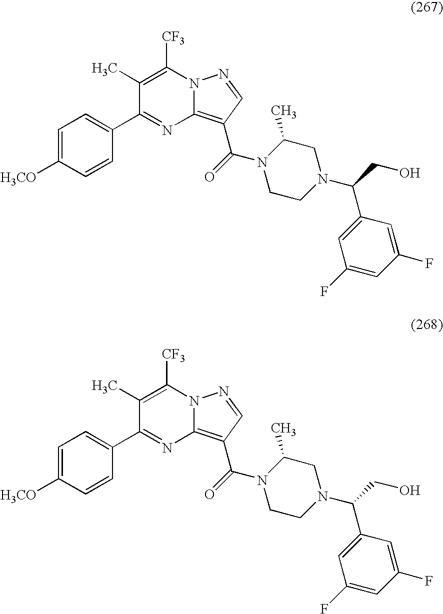

Piperazine 69 (50 mg, 0.12 mmol), glycolaldehyde dimer (8 mg, 0.06 mmol) and 3,5-difluorophenylboronic acid (20 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.91 and 1.93 min; MS (ES+) m/z 590; ¹H NMR δ_{H} (250 MHz, CDCl₃) 8.42 (1H, s), 7.47 (2H, d), 7.03 (2H, d), 6.67-6.82 (3H, m) 4.32-4.90 (1H, br s), 3.93 (3H, s), 3.88 (1H, obs m), 3.72 (1H, dd), 3.56 (1H, m), 3.20-3.45 (1H, br m), 2.84-3.17 (1H, br m), 2.75 (1H, t), 2.64 (1H, dd), 2.53 (3H, q), 2.49 (1H, obs m), 2.16 (0.5H, dd), 2.00 (0.5H, app td), 1.43 and 1.37 (3H, 2 d).

### Example 202

### [(2R)-4-[(1R)-1-(2-Chlorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (269) and [(2R)-4-[(1S)-1-(2-Chlorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (270)

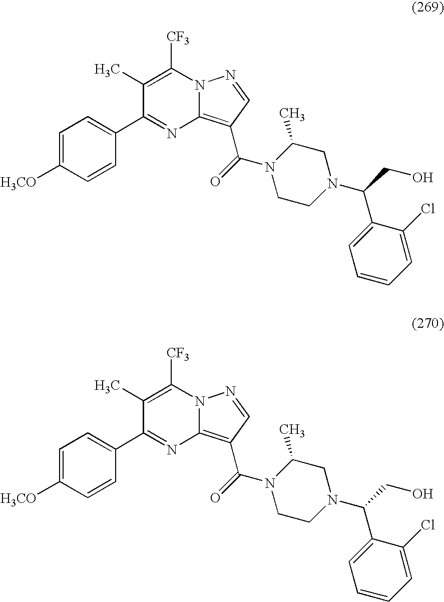

Piperazine 69 (50 mg, 0.12 mmol), glycolaldehyde dimer (8 mg, 0.06 mmol) and 2-chlorophenylboronic acid (20 mg, 0.13 mmol) were reacted in accordance with Method V to afford a 3:2 mixture of the title compounds after reverse phase preparative HPLC (5-95% MeCN in water) as a vitreous yellow solid: LC/MS t_{R} 1.83 and 1.88 min; MS (ES+) m/z 588, 590; ¹H NMR δ_{H} (250 MHz, DMSO-d₆, 368 K) 8.37 (1H, s), 7.54 (2H, d), 7.51 (1H, obs dd), 7.40 (1H, dd), 7.27 (2H, m), 7.08 (2H, d), 4.32-4.58 (1H, br s), 4.01 (2H, m), 3.88 (3H, s), 3.68-3.87 (2H, m), 3.16-3.35 (3H, m), 2.71 (1H, m), 2.44 (3H, q), 2.18-2.38 (1H, m), 1.28 and 1.22 (3H, 2 d).

### Example 203

### [(2R)-4-[(1R)-1-(3-Chlorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (271) and [(2R)-4-[(1S)-1-(3-Chlorophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (272)

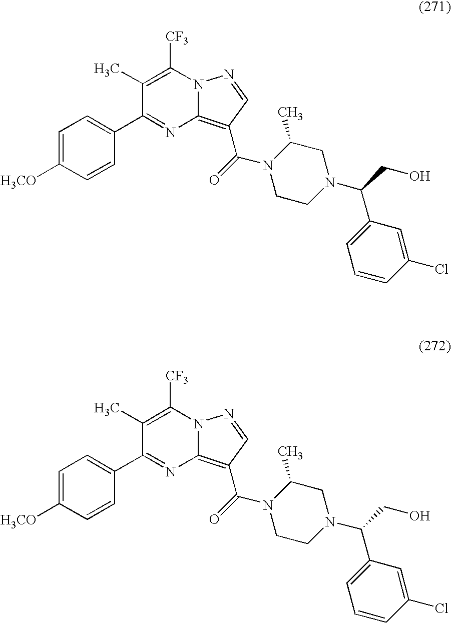

Piperazine 69 (50 mg, 0.12 mmol), glycolaldehyde dimer (8 mg, 0.06 mmol) and 3-chlorophenylboronic acid (20 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.79 and 1.82 min; MS (ES+) m/z 588, 590; ¹H NMR δ_{H} (250 MHz, CDCl₃) 8.46 and 8.45 (1H, 2 s), 7.45 (2H, d), 7.19-7.48 (3H, m), 7.01-7.14 (3H, m), 4.55-5.24 (1H, br s), 3.93 (3H, s), 3.74-4.48 (3H, m), 3.14-3.39 (2H, m), 3.04 (1H, d), 2.81 (1H, d), 2.68 (1H, br s), 2.58 (1H, obs m), 2.53 (3H, q), 2.11-2.42 (1H, m), 1.51 and 1.48 (3H, 2 d).

### Example 204

### [(2R)-4-[(1R)-2-Hydroxy-1-[3-(trifluoromethyl)phenyl]ethyl]-2-methyl-1-piperazinyl][5-(4-methoxyphenyl)-6-methyl-7-(trifluoromethyl)pyrazolo[1,5-a]pyrimidin-3-yl]methanone (273) and [(2R)-4-[(1S)-2-Hydroxy-1-[3-(trifluoromethyl)phenyl]ethyl]-2-methyl-1-piperazinyl][5-(4-methoxyphenyl)-6-methyl-7-(trifluoromethyl)pyrazolo[1,5-a]pyrimidin-3-yl]methanone (274)

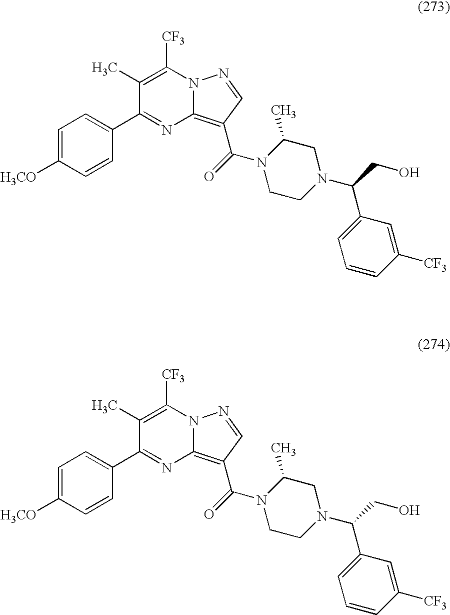

Piperazine 69 (50 mg, 0.12 mmol), glycolaldehyde dimer (8 mg, 0.06 mmol) and 3-(trifluoromethyl)phenylboronic acid (24 mg, 0.13 mmol) were reacted in accordance with Method V to afford a 1:1 mixture of the title compounds after reverse phase preparative HPLC (5-95% MeCN in water) and column chromatography (SCX silica gel, MeOH then NH₃ in MeOH, 2 M) as a vitreous yellow solid: LC/MS t_{R} 1.94 min; MS (ES+) m/z 622; ¹H NMR δ_{H} (250 MHz, CDCl₃) 8.40 and 8.39 (1H, 2 s), 7.59 (1H, d), 7.41-7.51 (4H, m), 7.37 (1H, d), 7.02 (2H, d), 4.10-4.98 (1H, br s), 3.95 (1H, obs m), 3.92 (3H, s), 3.90 (1H, obs m), 3.76 (1H, dd), 3.65 (1H, dd), 3.44 (1H, br s), 2.97 (1H, br d), 2.78 (1H, br t), 2.53 (3H, q), 2.35-2.65 (2H, m), 2.10 (0.5H, dd), 1.95 (0.5H, app td), 1.45 and 1.38 (3H, 2 d).

### Example 205

### [(2R)-4-[(1R)-2-Hydroxy-1-[2-(trifluoromethoxy)phenyl]ethyl]-2-methyl-1-piperazinyl][5-(4-methoxyphenyl)-6-methyl-7-(trifluoromethyl)pyrazolo[1,5-a]pyrimidin-3-yl]methanone (275) and [(2R)-4-[(1S)-2-Hydroxy-1-[2-(trifluoromethoxy)phenyl]ethyl]-2-methyl-1-piperazinyl][5-(4-methoxyphenyl)-6-methyl-7-(trifluoromethyl)pyrazolo[1,5-a]pyrimidin-3-yl]methanone (276)

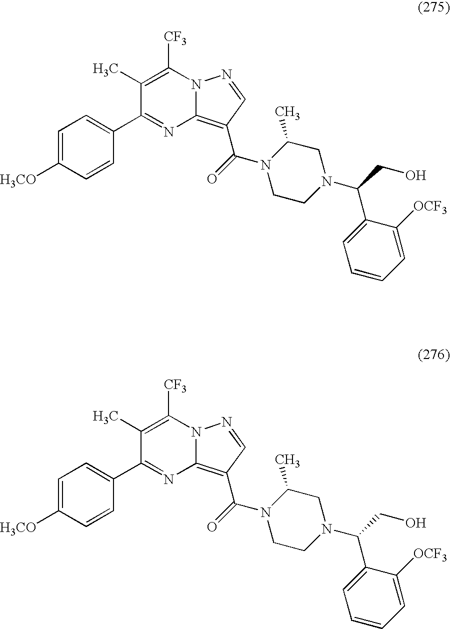

Piperazine 69 (50 mg, 0.12 mmol), glycolaldehyde dimer (8 mg, 0.06 mmol) and 2-(trifluoromethoxy)phenylboronic acid (26 mg, 0.13 mmol) were reacted in accordance with Method V to afford a 1:1 mixture of the title compounds after reverse phase preparative HPLC (5-95% MeCN in water) and column chromatography (SCX silica gel, MeOH then NH₃ in MeOH, 2 M) as a vitreous yellow solid: LC/MS t_{R} 2.00 and 2.05 min; MS (ES+) m/z 638; ¹H NMR δ_{H} (360 MHz, CDCl₃) 8.42 and 8.41 (1H, 2 s), 7.46 (2H, d), 7.38 (2H, m), 7.30 (2H, m), 7.03 (2H, d), 4.18-4.78 (2H, br m), 3.93 (3H, s), 3.85-4.09 (2H, obs m), 3.74 (1H, t), 2.97-3.25 (2H, m), 2.86 (1H, m), 2.53 (3H, q), 2.40-2.71 (2H, m), 2.34 0.5H, d), 2.08 (0.5H, t), 1.46 and 1.43 (3H, 2 d).

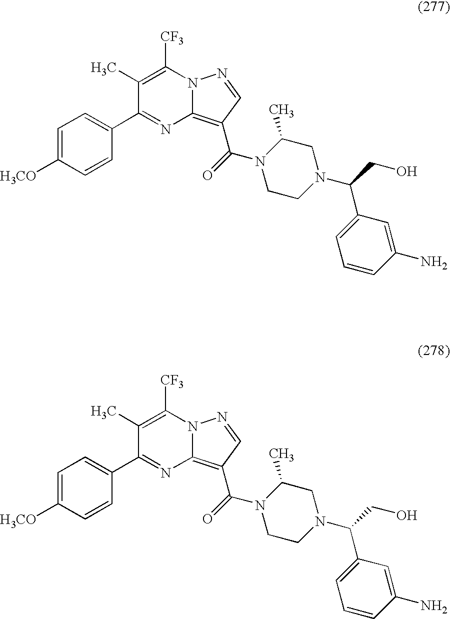

### Example 206

### N-(3-Boronophenyl)carbamic Acid, 1,1-Dimethylethyl Ester (279)

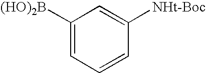

In a modified protocol to that of Fesik et al. (J. Am. Chem. Soc., 2001, 123, 10429), a stirred suspension of 3-aminophenylboronic acid monohydrate (300 mg, 1.94 mmol), DMAP (24 mg, 0.19 mmol) and MgSO₄ (200 mg) in MeCN (20 mL) was treated with di-tert-butyl dicarbonate (845 mg, 3.87 mmol) and stirred at rt for 18 h. Reaction progress was monitored by LC/MS. On completion the solvent was removed in vacuo and the residue partitioned between EtOAc (50 mL) and 0.5 M HCl (25 mL). The organic phase was isolated, washed with 0.5 M HCl (2×25 mL) and vigorously stirred for 1 h over 1 M NaOH (30 mL). The phases were separated, the aqueous extracted with EtOAc (2×25 mL) and the combined organic phases dried (MgSO₄), filtered and reduced in vacuo. The residue thus obtained was triturated with DCM (10 mL) and filtered. The filtrate was diluted with heptane (10 mL) and the resultant precipitate isolated by filtration to afford the title compound as an off-white solid: LC/MS t_{R} 1.58 min; MS (ES+) m/z 479, 680; ¹H NMR δ_{H} (250 MHz, CDCl₃) 7.81-7.98 (2H, m), 7.45 (1H, t), 6.68 (1H, s), 1.57 (9H, s).

### Example 207

### N-[3-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]carbamic Acid, 1,1-Dimethylethyl Ester (280) and N-[3-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]carbamic Acid, 1,1-Dimethylethyl Ester (281)

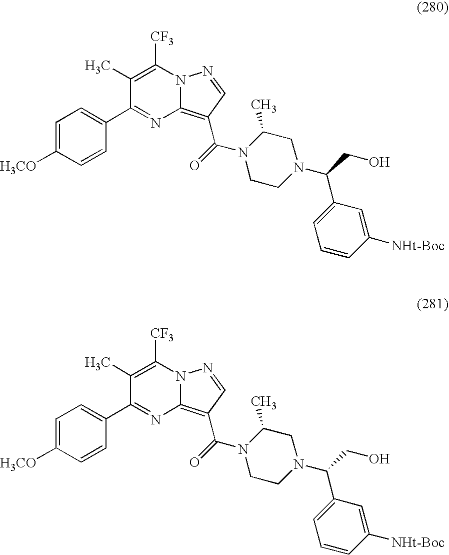

Piperazine 69 (200 mg, 0.46 mmol), glycolaldehyde dimer (30 mg, 0.26 mmol) and boronic acid 279 (120 mg, 0.50 mmol) were reacted in accordance with Method V to afford a 1:1 mixture of the title compounds after column chromatography (silica gel, 0-2% MeOH in DCM) as a vitreous yellow solid: LC/MS t_{R} 1.77 and 1.79 min; MS (ES+) m/z 669; ¹H NMR δ_{H} (360 MHz, CDCl₃) 8.40 and 8.39 (1H, 2 s), 7.46 (2H, d), 7.24 (3H, obs m), 7.02 (2H, d), 6.81 (1H, m), 6.57 (1H, br s), 4.08-5.18 (2H, br m), 3.92 (3H, s), 3.91 (1H, obs m), 3.70 (1H, d), 3.61 (1H, br s), 3.38 (1H, br s), 2.53 (3H, q), 2.39-3.02 (4H, m), 1.91-2.18 (1H, m), 1.51 (9H, s), 1.44 and 1.39 (3H, 2 d).

### Example 208

### [(2R)-4-[(1R)-1-(3-Aminophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (277) and [(2R)-4-[(1S)-1-(3-Aminophenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (278)

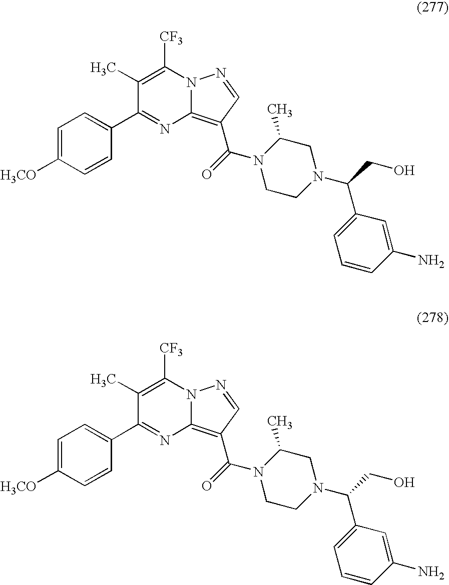

A 1:1 mixture of tert-butyl carbamates 280 and 281 (26 mg, 0.039 mmol) was treated with TFA in accordance with Method O to afford a 1:1 mixture of the title compounds as a glassy orange solid: LC/MS t_{R} 1.49 min; MS (ES+) m/z 569; ¹H NMR δ_{H} (360 MHz, CDCl₃) 8.40 (1H, s), 7.46 (2H, d), 7.09 (1H, t), 7.03 (2H, d), 6.62 (1H, dd), 6.52 (1H, d), 6.47 (1H, s), 4.08-4.97 (1H, br s), 3.92 (3H, s), 3.90 (1H, obs m), 3.18-3.78 (5H, m), 2.53 (3H, q), 2.41-2.98 (4H, m), 2.14 (0.5H, dd), 1.99 (0.5H, app td), 1.43 and 1.38 (3H, 2 d).

### Example 209

### [(2R)-4-[(1R)-1-[3-(Dimethylamino)phenyl]-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (282) and [(2R)-4-[(1S)-1-[3-(Dimethylamino)phenyl]-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (283)

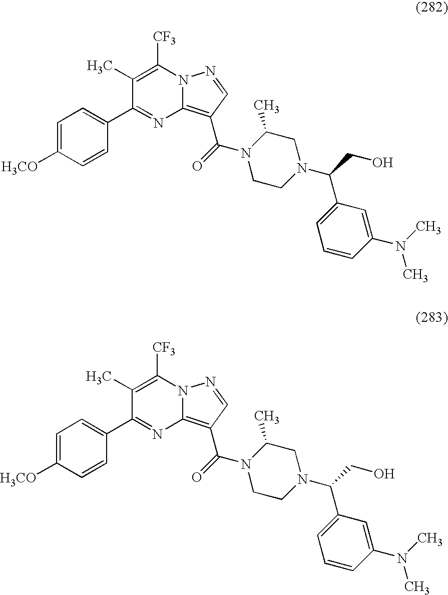

Piperazine 69 (50 mg, 0.12 mmol), glycolaldehyde dimer (8 mg, 0.06 mmol) and 3-(dimethylamino)phenylboronic acid (21 mg, 0.13 mmol) were reacted in accordance with Method V to afford a 1:1 mixture of the title compounds. Purification by column chromatography (silica gel, 0-5% MeOH in DCM) and reverse phase preparative HPLC (5-95% MeCN in water) afforded a 4:1 mixture of the title compounds as a vitreous yellow solid: LC/MS t_{R} 1.63 min; MS (ES+) m/z 597; ¹H NMR δ_{H} (360 MHz, CDCl₃) 8.40 and 8.39 (1H, 2 s), 7.48 (2H, d), 7.18 (1H, t), 7.02 (2H, d), 6.68 (1H, dd), 6.45-6.56 (2H, m), 4.20-5.00 (1H, br s), 3.95 (1H, obs m), 3.92 (3H, s), 3.71 (1H, m), 3.57 (1H, m), 3.15-3.52 (1H, m), 2.95 (6H, s), 2.62-2.99 (3H, m), 2.54 (3H, q), 2.50 (1H, obs m), 2.16 (0.5H, dd), 2.01 (0.5H, t), 1.45 and 1.39 (3H, 2 d).

### Example 210

### [(2R)-4-[(1R)-2-Hydroxy-1-(2,3,6-trifluorophenyl)ethyl]-2-methyl-1-piperazinyl][5-(4-methoxyphenyl)-6-methyl-7-(trifluoromethyl)pyrazolo[1,5-a]pyrimidin-3-yl]methanone (284) and [(2R)-4-[(1S)-2-Hydroxy-1-(2,3,6-trifluorophenyl)ethyl]-2-methyl-1-piperazinyl][5-(4-methoxyphenyl)-6-methyl-7-(trifluoromethyl)pyrazolo[1,5-a]pyrimidin-3-yl]methanone (285)

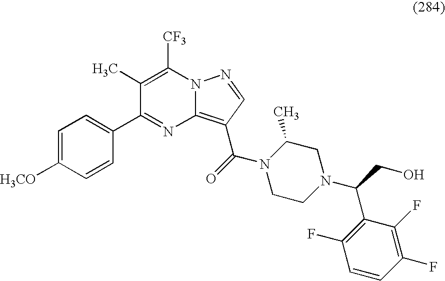

Piperazine 69 (50 mg, 0.12 mmol), glycolaldehyde dimer (8 mg, 0.06 mmol) and 2,3,6-trifluorophenylboronic acid (22 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 2.08 and 2.15 min; MS (ES+) m/z 608, 630; ¹H NMR δ_{H} (250 MHz, CDCl₃) 8.42 (1H, s), 7.45 (2H, d), 7.11 (1H, m), 7.03 (2H, d), 6.83 (1H, m), 4.46-5.16 (1H, br s), 4.34 (2H, m), 3.94 (3H, s), 3.92 (1H, obs m), 3.66 (1H, d), 3.41 (1H, br m), 2.70-3.11 (2H, m), 2.53 (3H, q), 2.43-2.71 (2H, m), 2.17 (0.5H, d), 2.00 (0.5H, t), 1.41 (3H, d).

### Example 211

### [(2R)-4-[(1R)-2-Hydroxy-1-[3-(trifluoromethoxy)phenyl]ethyl]-2-methyl-1-piperazinyl][5-(4-methoxyphenyl)-6-methyl-7-(trifluoromethyl)pyrazolo[1,5-a]pyrimidin-3-yl]methanone (286) and [(2R)-4-[(1S)-2-Hydroxy-1-[3-(trifluoromethoxy)phenyl]ethyl]-2-methyl-1-piperazinyl][5-(4-methoxyphenyl)-6-methyl-7-(trifluoromethyl)pyrazolo[1,5-a]pyrimidin-3-yl]methanone (287)

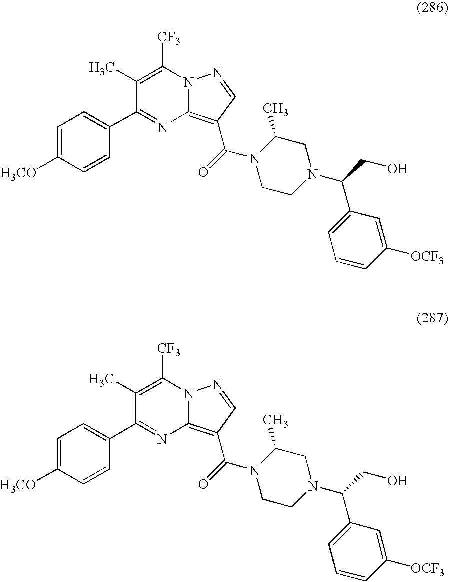

Piperazine 69 (50 mg, 0.12 mmol), glycolaldehyde dimer (8 mg, 0.06 mmol) and 3-(trifluoromethoxy)phenylboronic acid (26 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.96 min; MS (ES+) m/z 638; ¹H NMR δ_{H} (250 MHz, CDCl₃) 8.41 and 8.40 (1H, 2 s), 7.47 (2H, d), 7.36 (1H, t), 7.18 (1H, d), 6.98-7.12 (4H, m), 4.32-4.94 (1H, br s), 3.92 (3H, s), 3.85-4.04 (2H, obs m), 3.75 (1H, app dt), 3.62 (1H, m), 3.21-3.53 (1H, br m), 2.96 (1H, br d), 2.76 (1H, t), 2.61 (1H, dd), 2.52 (3H, q), 2.44 (1H, app td), 2.14 (0.5H, dd), 1.96 (0.5H, app td), 1.44 and 1.38 (3H, 2 d).

### Example 212

### N-[3-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]-2-methylpropaneamide (288) and N-[3-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]-2-methylpropaneamide (289)

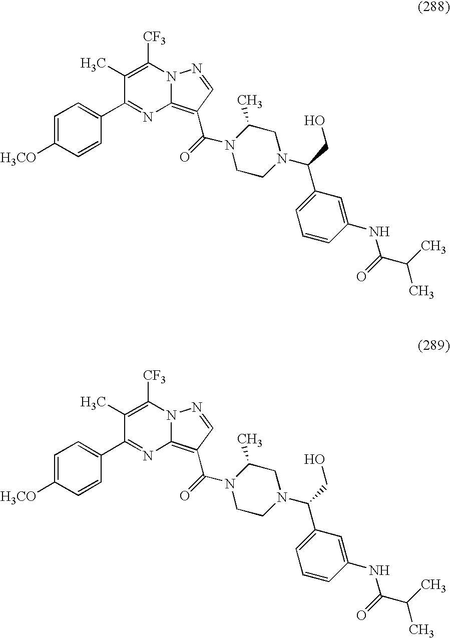

A suspension of a 1:1 mixture of anilines 277 and 278 (30 mg, 0.053 mmol) and NaHCO₃ (6.7 mg, 0.080 mmol) in DCM (2 mL) was cooled to 0° C. and treated with isobutyryl chloride (6.1 μL, 0.058 mmol, d 1.017). After stirring at rt for 1 h, the reaction mixture was diluted with DCM (10 mL) and water (10 mL) and the two phases separated. The organic phase was washed with water (2×10 mL) and brine (10 mL), dried (Na₂SO₄), filtered and reduced in vacuo. Column chromatography (silica gel, 0-3% MeOH in DCM) afforded a 1:1 mixture of the title compounds as a vitreous yellow solid: LC/MS t_{R} 1.64 min; MS (ES+) m/z 639, 661; ¹H NMR δ_{H} (360 MHz, CDCl₃) 8.37 and 8.36 (1H, 2 s), 7.42-7.58 (4H, m), 7.39 (1H, t), 7.23 (1H, t), 7.01 (2H, d), 6.86 (1H, br d), 4.07-4.99 (1H, br s), 3.92 (3H, s), 3.89 (1H, obs m), 3.52-3.74 (3H, m), 3.11-3.80 (1H, br s), 2.58-2.98 (3H, m), 2.53 (3H, m), 2.38-2.57 (2H, obs m), 2.09 (0.5H, d), 1.94 (0.5H, t), 1.43 and 1.37 (3H, 2 d), 1.22 (6H, d).

### Example 213

### N-[3-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]cyclopropanecarboxamide (290) and N-[3-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]cyclopropanecarboxamide (291)

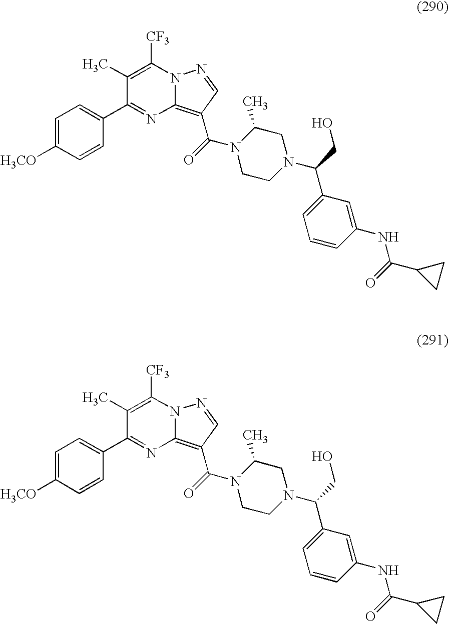

A suspension of a 1:1 mixture of anilines 277 and 278 (30 mg, 0.053 mmol) and NaHCO₃ (6.7 mg, 0.080 mmol) in DCM (2 mL) was cooled to 0° C. and treated with cyclopropanecarbonyl chloride (5.3 μL, 0.058 mmol, d 1.152). After stirring at rt for 1 h, the reaction mixture was diluted with DCM (10 mL) and water (10 mL) and the two phases separated. The organic phase was washed with water (2×10 mL) and brine (10 mL), dried (Na₂SO₄), filtered and reduced in vacuo. Column chromatography (silica gel, 0-3% MeOH in DCM) afforded a 1:1 mixture of the title compounds as a vitreous yellow solid: LC/MS t_{R} 1.61 min; MS (ES+) m/z 637, 659; ¹H NMR δ_{H} (360 MHz, CDCl₃) 8.363 and 8.358 (1H, 2 s), 7.97 (1H, br s), 7.45 (2H, d), 7.45 (1H, obs d), 7.38 (1H, t), 7.21 (1H, t), 7.00 (2H, d), 6.84 (1H, d), 4.12-4.96 (1H, br m), 3.91 (3H, s), 3.90 (1H, obs m), 3.50-3.71 (3H, m), 3.07-3.76 (1H, br s), 2.82 (1H, br s), 2.73 (1H, br s), 2.59 (1H, dd), 2.53 (3H, q), 2.43 (1H, app td), 2.08 (0.5H, dd), 1.92 (0.5H, t), 1.51 (1H, m), 1.42 and 1.36 (3H, 2 d), 1.03 (2H, m), 0.78 (2H, m).

### Example 214

### N-[3-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]urea (292) and N-[3-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]urea (293)

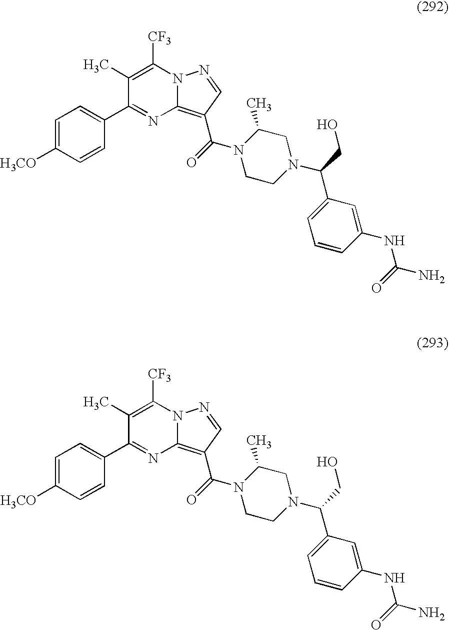

A solution of a 1:1 mixture of anilines 277 and 278 (30 mg, 0.053 mmol) in AcOH (0.5 mL) and water (0.5 mL) was treated with potassium cyanate (26 mg, 0.32 mmol) and stirred at rt for 16 h. Reaction progress was monitored by LC/MS. On completion the reaction was diluted with water (4 mL) and extracted into EtOAc (3×5 mL). The combined EtOAc phases were washed with satd NaHCO₃ (2×10 mL), dried (Na₂SO₄) and filtered and the filtrate reduced in vacuo. Column chromatography (silica gel, 0-10% MeOH in DCM) afforded a 1:1 mixture of the title compounds as a vitreous yellow solid: LC/MS t_{R} 3.07 and 3.09 min; MS (ES+) m/z 612, 634; ¹H NMR δ_{H} (360 MHz, CDCl₃) 8.322 and 8.315 (1H, 2 s), 7.99 (1H, s), 7.44 (2H, d), 7.30 (1H, br t), 7.05-7.18 (2H, m), 7.00 (2H, d), 6.74 (1H, br d), 5.07 (2H, br s), 4.00-4.90 (1H, br s), 3.89 (3H, s), 3.84 (2H, obs m), 3.61 (1H, m), 3.49 (1H, m), 3.07 (1H, br s), 2.85 (1H, br s), 2.70 (1H, br d), 2.54 (1H, obs d), 2.51 (3H, q), 2.39 (1H, app td), 2.06 (0.5H, dd), 1.92 (0.5H, t), 1.41 and 1.34 (3H, 2 d).

### Example 215

### [(2R)-4-[(1R)-2-Hydroxy-1-[2-(trifluoromethyl)phenyl]ethyl]-2-methyl-1-piperazinyl][5-(4-methoxyphenyl)-6-methyl-7-(trifluoromethyl)pyrazolo[1,5-a]pyrimidin-3-yl]methanone (294) and [(2R)-4-[(1S)-2-Hydroxy-1-[2-(trifluoromethyl)phenyl]ethyl]-2-methyl-1-piperazinyl][5-(4-methoxyphenyl)-6-methyl-7-(trifluoromethyl)pyrazolo[1,5-a]pyrimidin-3-yl]methanone (295)

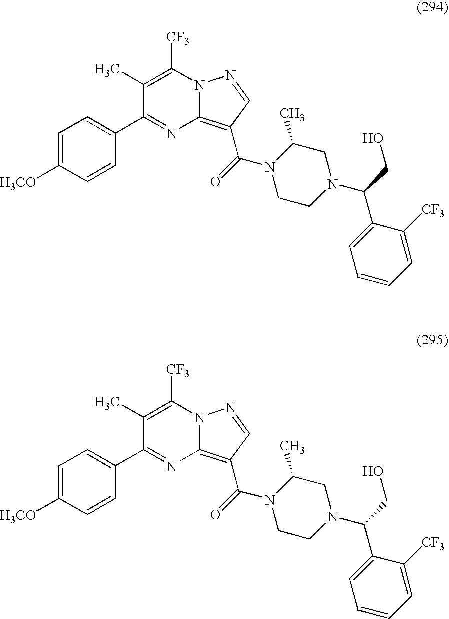

Piperazine 69 (50 mg, 0.12 mmol), glycolaldehyde dimer (8 mg, 0.06 mmol) and 2-(trifluoromethyl)phenylboronic acid (24 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 2.13 min; MS (ES+) m/z 622; ¹H NMR δ_{H} (250 MHz, CDCl₃) 8.45 and 8.44 (1H, 2 s), 7.79 (1H, br d), 7.69 (1H, d), 7.56 (1H, t), 7.49 and 7.47 (2H, 2 d), 7.41 (1H, t), 7.02 and 7.01 (2H, 2 d), 4.29-5.11 (1H, br m), 3.92 (3H, s), 3.70-3.96 (4H, m), 2.98-3.38 (2H, br s), 2.54 (3H, m), 2.12-2.70 (4H, m), 1.46 (3H, d).

### Example 216

### [(2R)-4-[(1R)-1-(2,2-Difluoro-1,3-benzodioxol-4-yl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (296) [(2R)-4-[(1R)-1-(2,2-Difluoro-1,3-benzodioxol-4-yl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (297)

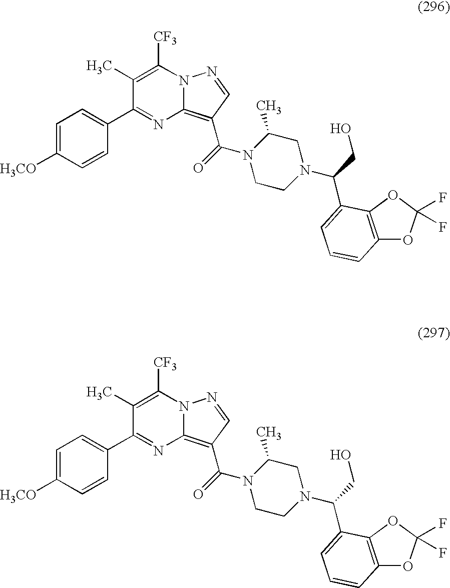

Piperazine 69 (50 mg, 0.12 mmol), glycolaldehyde dimer (8 mg, 0.06 mmol) and (2,2-difluoro-1,3-benzodioxol-4-yl)boronic acid (23 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 2.05 and 2.13 min; MS (ES+) m/z 634; ¹H NMR δ_{H} (250 MHz, CDCl₃) 8.394 and 8.389 (1H, 2 s), 7.46 (2H, d), 6.95-7.09 (4H, m), 6.85 (1H, t), 4.24-5.05 (1H, br s), 4.06 (1H, m), 3.94 (1H, obs m), 3.93 (3H, s), 3.70 (1H, dd), 3.14-3.56 (1H, br s), 2.57-3.04 (3H, m), 2.53 (3H, q), 2.43 (1H, t), 2.12 (0.5H, dd), 1.96 (0.5H, app td), 1.44 and 1.40 (3H, 2 d).

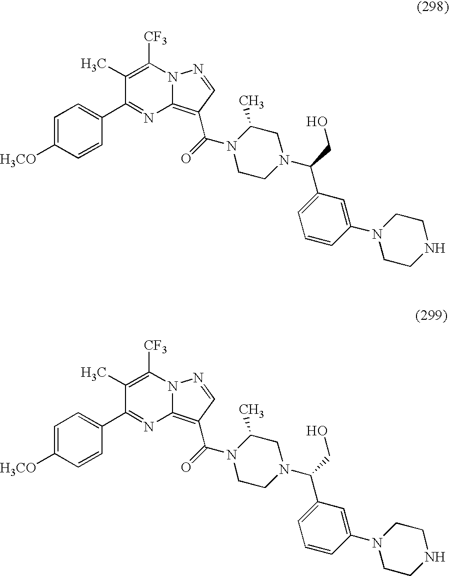

### Example 217

### 4-(3-Bromophenyl)-1-piperazinecarboxylic Acid, 1,1-Dimethylethyl Ester (300)

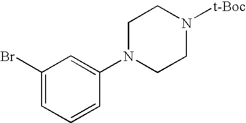

1-(3-Bromophenyl)piperazine (0.50 g, 2.07 mmol) was treated with di-tert-butyl dicarbonate (0.50 g, 2.28 mmol) employing the procedure of Method U to afford the title compound as a colorless viscous oil: LC/MS t_{R} 2.45 min; MS (ES+) m/z 241, 243, 285, 287, 341, 343; ¹H NMR δ_{H} (250 MHz, CDCl₃) 7.13 (1H, t), 7.05 (1H, t), 7.00 (1H, d), 6.85 (1H, dd), 3.58 (4H, m), 3.14 (4H, m), 1.49 (9H, s).

### Example 218

### 4-(3-Boronophenyl)-1-piperazinecarboxylic Acid, 1,1-Dimethylethyl Ester (301)

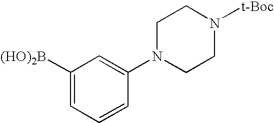

In accordance with the procedure of Li and Nelson et al. (J. Org. Chem., 2002, 67, 5394), a solution of aryl bromide 300 (0.25 g, 0.73 mmol) and triisopropyl borate (165 μL, 0.88 mmol, d 0.815) in PhMe (4 mL) and THF (1 mL) at −78° C. was treated with n-BuLi (0.55 mL, 0.88 mmol, 1.6 M in hexanes). After stirring for 30 min at this temperature, the reaction mixture was allowed to warm to −20° C. and quenched by the addition of satd NH₄Cl (20 mL). The biphasic mixture was allowed to warm to rt and extracted with EtOAc (3×20 mL). The combined EtOAc phases were dried (Na₂SO₄) and filtered and the filtrate reduced in vacuo. Column chromatography (silica gel, 0-3% MeOH in DCM) afforded the title compound as a brown crystalline solid: LC/MS t_{R} 1.67 min; MS (ES+) m/z 307.

### Example 219

### 4-[3-[(1R)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]-1-piperazinecarboxylic Acid, 1,1-Dimethylethyl Ester (302) and -[3-[(1S)-2-Hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]-1-piperazinecarboxylic Acid, 1,1-Dimethylethyl Ester (303)

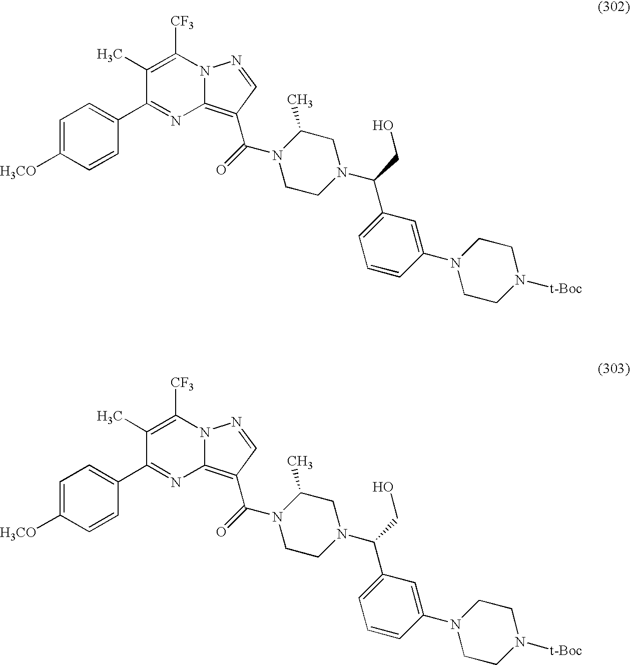

Piperazine 69 (50 mg, 0.12 mmol), glycolaldehyde dimer (8 mg, 0.06 mmol) and boronic acid 301 (39 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.82 and 1.84 min; MS (ES+) m/z 682, 738.

### Example 220

### [(2R)-4-[(1R)-2-Hydroxy-1-[3-(1-piperazinyl)phenyl]ethyl]-2-methyl-1-piperazinyl][5-(4-methoxyphenyl)-6-methyl-7-(trifluoromethyl)pyrazolo[1,5-a]pyrimidin-3-yl]methanone (298) and [(2R)-4-[(1S)-2-Hydroxy-1-[3-(1-piperazinyl)phenyl]ethyl]-2-methyl-1-piperazinyl][5-(4-methoxyphenyl)-6-methyl-7-(trifluoromethyl)pyrazolo[1,5-a]pyrimidin-3-yl]methanone (299)

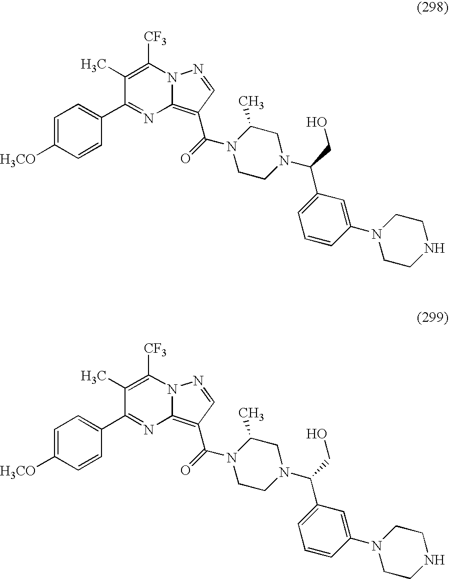

The 1:1 mixture of tert-butyl carbamates 302 and 303 (19.2 mg, 0.026 mmol) was treated with a solution of HCl in 1,4-dioxane (2 mL, 4 M) and stirred for 1 h at rt. Reaction progress was monitored by LC/MS. On completion, the solvent was removed in vacuo to afford a 1:1 mixture of the HCl salts of the title compounds as a vitreous yellow solid: LC/MS t_{R} 1.25 min; MS (ES+) m/z 319.5, 340, 638; ¹H NMR δ_{H} (250 MHz, CD₃OD) 8.45 and 8.42 (1H, 2 s), 7.57 (2H, d), 7.21-7.54 (2H, m), 7.08-7.20 (3H, m), 6.64-7.07 (1H, br m), 4.99 (1H, obs br s), 4.06-4.51 (3H, m), 3.93 (3H, s), 3.87-3.98 (1H, obs m), 3.51 (4H, m), 3.45-3.77 (3H, m), 3.38 (4H, m), 3.96-3.28 (2H, m), 2.51 (3H, q), 1.62 and 1.48 (3H, 2 d).

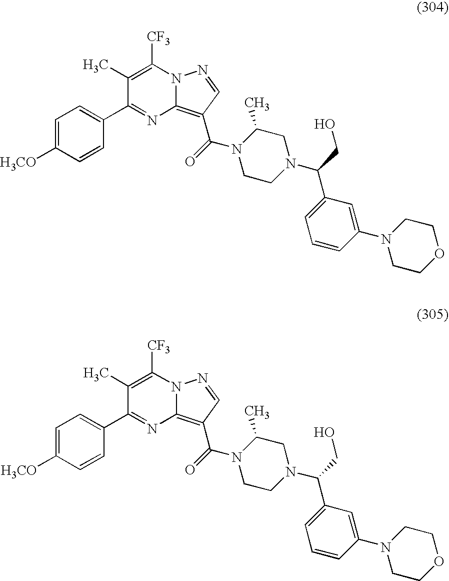

### Example 221

### 3-(4-Morpholinyl)phenylboronic Acid (306)

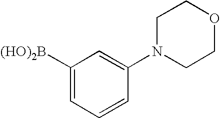

In accordance with the procedure of Li and Nelson et al. (J. Org. Chem., 2002, 67, 5394), a solution of 4-(3-bromophenyl)morpholine (0.50 g, 2.06 mmol) and triisopropyl borate (0.59 mL, 2.51 mmol, d 0.815) in PhMe (8 mL) and THF (2 mL) at −78° C. was treated with n-BuLi (1.55 mL, 2.48 mmol, 1.6 M in hexanes). After stirring for 30 min at this temperature, the reaction mixture was allowed to warm to −20° C. and quenched by the addition of satd NH₄Cl (40 mL). The biphasic mixture was allowed to warm to rt and extracted with EtOAc (3×40 mL). The combined EtOAc phases were dried (Na₂SO₄) and filtered and the filtrate reduced in vacuo. Column chromatography (silica gel, 0-3% MeOH in DCM) afforded the title compound as a white powder: LC/MS t_{R} 0.93 min; MS (ES+) m/z 208.

### Example 222

### [(2R)-4-[(1R)-2-Hydroxy-1-[3-(4-morpholinyl)phenyl]ethyl]-2-methyl-1-piperazinyl][5-(4-methoxyphenyl)-6-methyl-7-(trifluoromethyl)pyrazolo[1,5-a]pyrimidin-3-yl]methanone (304) and [(2R)-4-[(1S)-2-Hydroxy-1-[3-(4-morpholinyl)phenyl]ethyl]-2-methyl-1-piperazinyl][5-(4-methoxyphenyl)-6-methyl-7-(trifluoromethyl)pyrazolo[1,5-a]pyrimidin-3-yl]methanone (305)

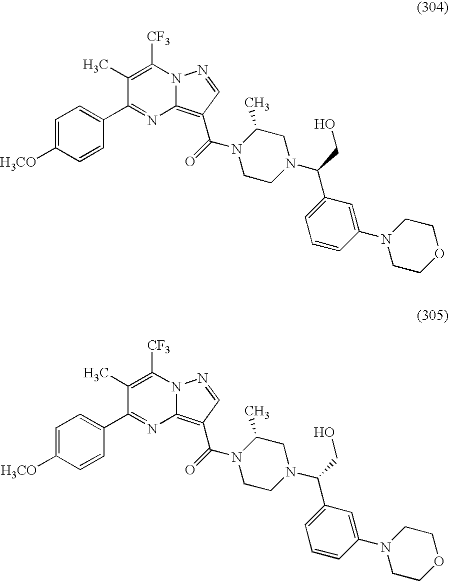

Piperazine 69 (50 mg, 0.12 mmol), glycolaldehyde dimer (8 mg, 0.06 mmol) and boronic acid 306 (26 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.62 min; MS (ES+) m/z 639; ¹H NMR δ_{H} (250 MHz, CDCl₃) 8.39 and 8.38 (1H, 2 s), 7.48 (2H, d), 7.22 (1H, t), 7.02 (2H, d), 6.86 (1H, dd), 6.60-6.76 (2H, m), 4.14-5.18 (1H, br s), 3.93 (1H, obs m), 3.92 (3H, s), 3.86 (4H, m), 3.20-3.78 (4H, m), 3.14 (4H, m), 2.59-3.01 (3H, m), 2.54 (3H, q), 2.35-2.57 (1H, obs m), 2.16 (0.5H, d), 1.99 (0.5H, app td), 1.45 and 1.39 (3H, 2 d).

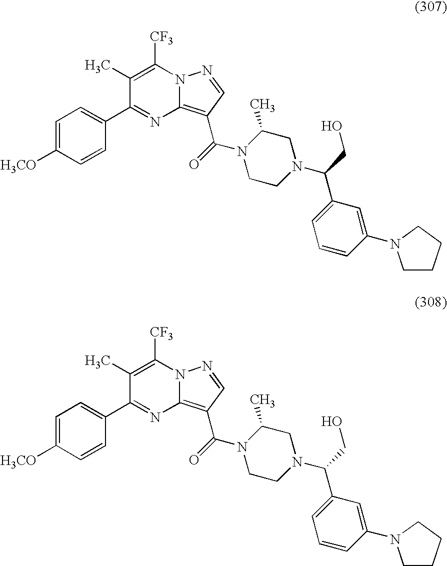

### Example 223

### 3-(1-Pyrrolidinyl)phenylboronic Acid (309)

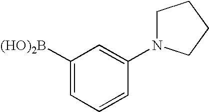

In accordance with the procedure of Li and Nelson et al. (J. Org. Chem., 2002, 67, 5394), a solution of 1-(3-bromophenyl)pyrrolidine (0.47 g, 2.06 mmol) and triisopropyl borate (0.59 mL, 2.51 mmol, d 0.815) in PhMe (8 mL) and THF (2 mL) at −78° C. was treated with n-BuLi (1.55 mL, 2.48 mmol, 1.6 M in hexanes). After stirring for 30 min at this temperature, the reaction mixture was allowed to warm to −20° C. and quenched by the addition of satd NH₄Cl (40 mL). The biphasic mixture was allowed to warm to rt and extracted with EtOAc (3×40 mL). The combined EtOAc phases were dried (Na₂SO₄) and filtered and the filtrate reduced in vacuo. Column chromatography (silica gel, 0-3% MeOH in DCM) afforded the title compound as a white powder: LC/MS t_{R} 0.96 min; MS (ES+) m/z 192.

### Example 224

### [(2R)-4-[(1R)-2-Hydroxy-1-[3-(1-pyrrolidinyl)phenyl]ethyl]-2-methyl-1-piperazinyl][5-(4-methoxyphenyl)-6-methyl-7-(trifluoromethyl)pyrazolo[1,5-a]pyrimidin-3-yl]methanone (307) and [(2R)-4-[(1S)-2-Hydroxy-1-[3-(1-pyrrolidinyl)phenyl]ethyl]-2-methyl-1-piperazinyl][5-(4-methoxyphenyl)-6-methyl-7-(trifluoromethyl)pyrazolo[1,5-a]pyrimidin-3-yl]methanone (308)

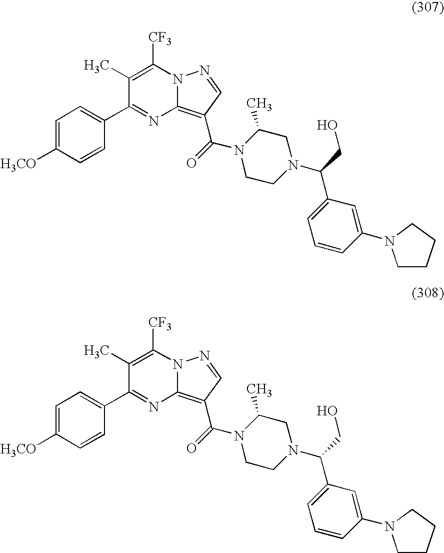

Piperazine 69 (50 mg, 0.12 mmol), glycolaldehyde dimer (8 mg, 0.06 mmol) and boronic acid 309 (24 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.75 min; MS (ES+) m/z 623; ¹H NMR δ_{H} (250 MHz, CDCl₃) 8.39 and 8.38 (1H, 2 s), 7.48 (2H, d), 7.16 (1H, t), 7.02 (2H, d), 6.51 (1H, dd), 6.42 (1H, d), 6.33 (1H, d), 4.08-4.92 (1H, br s), 3.92 (3H, s), 3.83-4.04 (1H, obs m), 3.70 (1H, m), 3.58 (1H, m), 3.27 (4H, m), 3.08-3.35 (1H, obs br s), 2.60-3.03 (3H, m), 2.54 (3H, q), 2.38-2.59 (1H, obs m), 2.01 (4H, m), 1.82-2.25 (2H, m), 1.45 and 1.40 (3H, 2 d).

### Example 225

### [(2R)-4-[(1R)-1-(3-Furanyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (310) and [(2R)-4-[(1S)-1-(3-Furanyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (311)

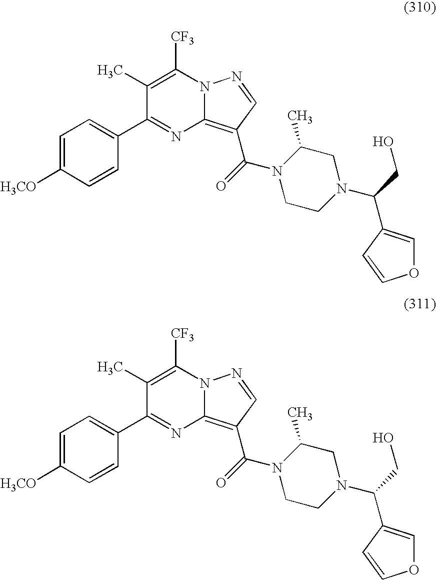

Piperazine 69 (50 mg, 0.12 mmol), glycolaldehyde dimer (8 mg, 0.06 mmol) and 3-furanylboronic acid (14 mg, 0.13 mmol) were reacted in accordance with Method V and purified by column chromatography (silica gel, 60% EtOAc in heptanes) to afford a 1:1 mixture of the title compounds as a vitreous yellow solid: LC/MS t_{R} 3.10 min; MS (ES+) m/z 544, 566; ¹H NMR δ_{H} (250 MHz, CDCl₃) 8.42 (1H, s), 7.46 (2H, d), 7.40 (1H, d), 7.23 (1H, s), 7.03 (2H, d), 6.19 (1H, d), 4.04-4.82 (1H, br m), 3.93 (3H, s), 3.58-3.82 (3H, m), 2.83-3.57 (2H, br m), 2.72 (1H, m), 2.53 (3H, q), 2.43-2.64 (2H, obs m), 2.19 (0.5H, dd), 2.03 (0.5H, app td), 1.41 and 1.38 (3H, 2 d).

### Example 226

### 1-[4-Fluoro-3-[(1R)-2-hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]ethanone (312) and 1-[4-Fluoro-3-[(1S)-2-hydroxy-1-[(3R)-4-[[5-(4-methoxyphenyl)-6-methyl-7-(trifluoromethyl)pyrazolo[1,5-a]pyrimidin-3-yl]carbonyl]-3-methyl-1-piperazinyl]ethyl]phenyl]ethanone (313)

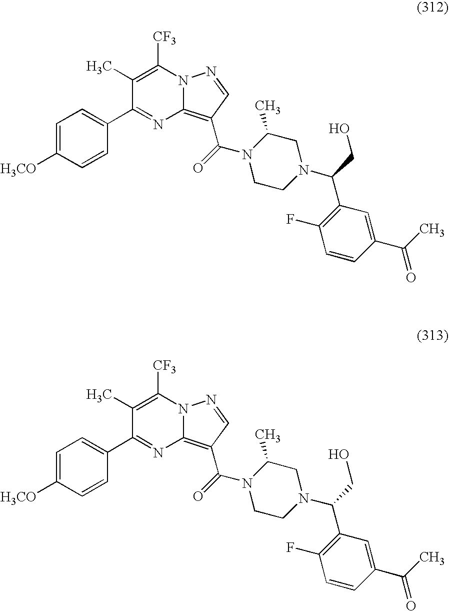

Piperazine 69 (50 mg, 0.12 mmol), glycolaldehyde dimer (8 mg, 0.06 mmol) and 5-acetyl-2-fluorophenylboronic acid (21 mg, 0.13 mmol) were reacted in accordance with Method V to afford a 1:1 mixture of the title compounds after reverse phase preparative HPLC (5-95% MeCN in water) as a vitreous yellow solid: LC/MS t_{R} 1.84 min; MS (ES+) m/z 614, 636; ¹H NMR δ_{H} (250 MHz, CDCl₃) 8.40 and 8.39 (1H, 2 s), 7.75-7.96 (2H, m), 7.46 (2H, d), 7.15 (1H, t), 7.02 (2H, d), 4.29-4.88 (1H, br s), 4.00-4.23 (2H, m), 3.94 (3H, s), 3.70 (1H, m), 3.17-3.56 (1H, br s), 2.69-3.05 (2H, m), 2.59 (3H, s), 2.53 (3H, q), 2.32-2.66 (2H, m), 2.08 (0.5H, d), 1.91 (0.5H, t), 1.45 and 1.43 (3H, 2 d).

### Example 227

### [(2R)-4-[(1R)-2-Hydroxy-1-(2,3,5-trifluorophenyl)ethyl]-2-methyl-1-piperazinyl][5-(4-methoxyphenyl)-6-methyl-7-(trifluoromethyl)pyrazolo[1,5-a]pyrimidin-3-yl]methanone (314) and [(2R)-4-[(1S)-2-Hydroxy-1-(2,3,5-trifluorophenyl)ethyl]-2-methyl-1-piperazinyl][5-(4-methoxyphenyl)-6-methyl-7-(trifluoromethyl)pyrazolo[1,5-a]pyrimidin-3-yl]methanone (315)

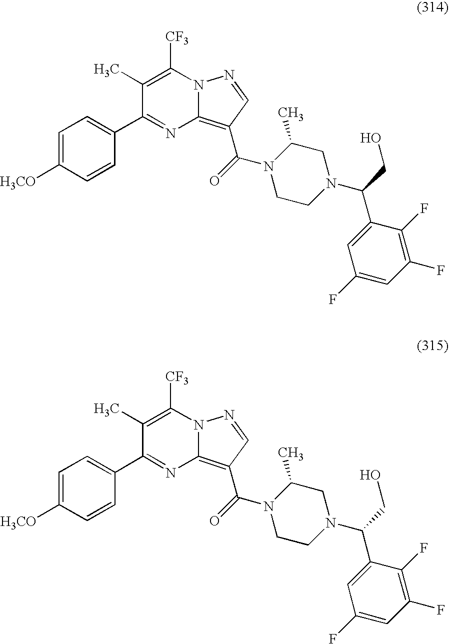

Piperazine 69 (50 mg, 0.12 mmol), glycolaldehyde dimer (8 mg, 0.06 mmol) and 2,3,5-trifluorophenylboronic acid (22 mg, 0.13 mmol) were reacted in accordance with Method V to afford a 1:1 mixture of the title compounds after column chromatography (silica gel, 0-2% MeOH in DCM; repeated using 60% EtOAc in heptanes) as a vitreous yellow solid: LC/MS t_{R} 2.13 and 2.15 min; MS (ES+) m/z 608, 630; ¹H NMR δ_{H} (250 MHz, CDCl₃) 8.44 (1H, s), 7.47 (2H, d), 7.04 (2H, d), 6.90 (1H, m), 6.70 (1H, m), 4.12 (1H, m), 3.97-4.76 (1H, br s), 3.93 (3H, s), 3.90 (1H, obs m), 3.66 (1H, br d), 3.42 (1H, br s), 2.54 (3H, q), 2.35-3.04 (3H, m), 2.15 (0.5H, dd), 2.00 (0.5H, app td), 1.42 and 1.39 (3H, 2 d).

### Example 228

### (2R)-4-[(1R)-1-(2,6-Difluoro-3-methoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (316) and (2R)-4-[(1S)-1-(2,6-Difluoro-3-methoxyphenyl)-2-hydroxyethyl]-2-methyl-1-piperazinyl][5-(4-methoxyphenyl)-6-methyl-7-(trifluoromethyl)pyrazolo[1,5-a]pyrimidin-3-yl]methanone (317)

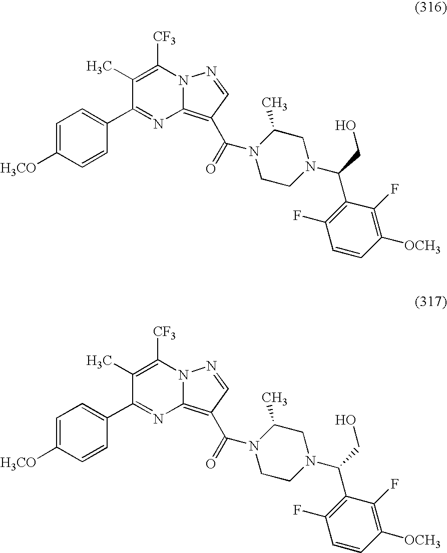

Piperazine 69 (50 mg, 0.12 mmol), glycolaldehyde dimer (8 mg, 0.06 mmol) and 2,6-difluoro-3-methoxyphenylboronic acid (24 mg, 0.13 mmol) were reacted in accordance with Method V to afford a 1:1 mixture of the title compounds after column chromatography (silica gel, 60% EtOAc in heptanes) as a vitreous yellow solid: LC/MS t_{R} 1.93 and 2.05 min; MS (ES+) m/z 620, 642; ¹H NMR δ_{H} (250 MHz, CDCl₃) 8.41 (1H, s), 7.46 (2H, d), 7.05 (2H, d), 6.74-6.92 (2H, m), 3.98-5.05 (3H, m), 3.93 (3H, s), 3.87 (1H, obs m), 3.86 (3H, s), 3.65 (1H, br s), 3.39 (1H, br s), 2.84-3.20 (2H, m), 2.57-2.80 (1H, m), 2.54 (3H, q), 2.33-2.56 (1H, obs m), 2.17 (0.5H, d), 2.00 (0.5H, t), 1.42 and 1.40 (3H, 2 d).

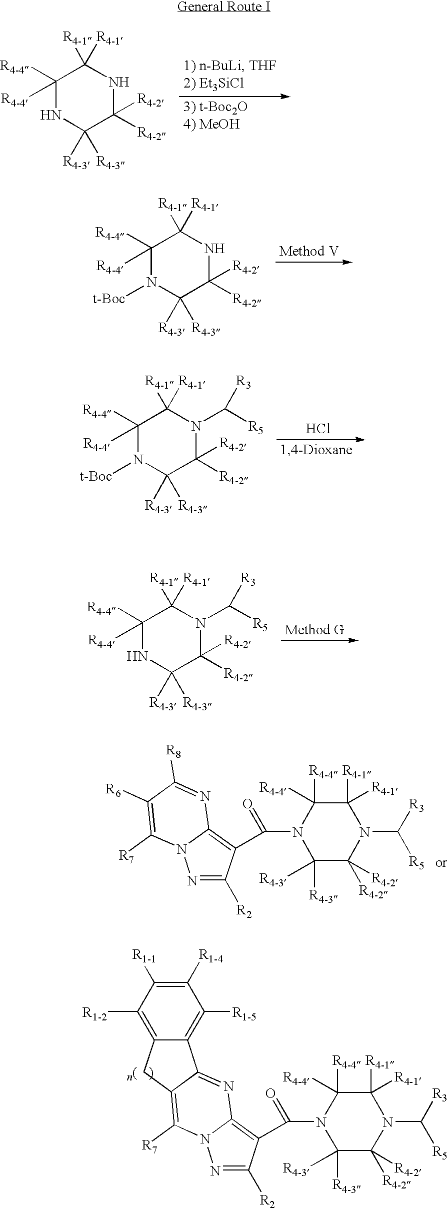

### Description of Method Used in General Route I

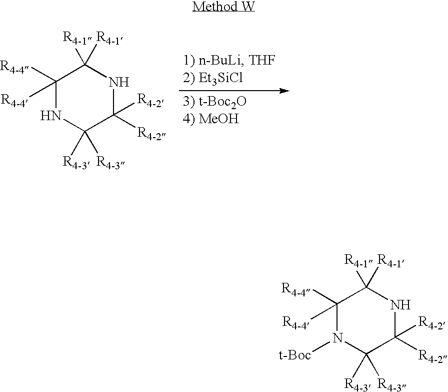

In an adaptation of the procedure of Wang, Zhang and Meanwell (J. Org. Chem., 2000, 65, 4740-4742), a solution of n-BuLi (2.2 equiv) was added dropwise over 5-10 min to a stirred solution of the piperazine (1 equiv) in THF (60 vol). After 30 min, Et₃SiCl (1.1 equiv) was added and the reaction stirred a further 1 h before treating with di-tert-butyl dicarbonate (0.96 equiv). After 30 min, the reaction was quenched carefully with MeOH (15 vol) and the solvents were evaporated. Column chromatography (silica gel, 0-4% MeOH in DCM) gave the desired product.

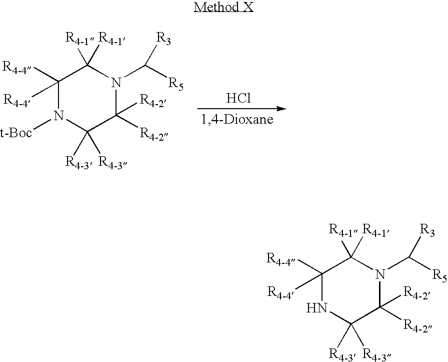

A solution of the tert-butyl carbamate (1 equiv) in 1,4-dioxane (10 vol) was treated with a solution of HCl in 1,4-dioxane (50 vol, 4 M) and stirred for 1 h at rt. Reaction progress was monitored by LC/MS. On completion, the solvent was removed in vacuo to afford the desired product as the HCl salt.

### Examples of Compounds Prepared via General Route I

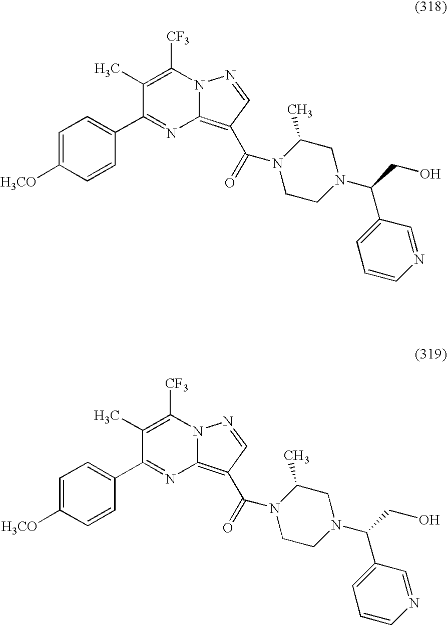

### Example 229

### (R)-2-Methyl-1-piperazinecarboxylic Acid, 1,1-Dimethylethyl Ester (320)

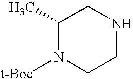

(R)-(−)-2-Methylpiperazine (0.30 g, 3.00 mmol) was treated with n-BuLi (2.60 mL, 6.60 mmol, 2.5 M in hexanes), Et₃SiCl (0.55 mL, 3.30 mmol, d 0.898) and di-tert-butyl dicarbonate (0.63 g, 2.88 mmol) in accordance with Method W to afford the title compound after column chromatography (silica gel, 0-4% MeOH in DCM) as a white wax: LC/MS t_{R} 0.95 min; MS (ES+) m/z 145, 201; ¹H NMR δ_{H} (250 MHz, CDCl₃) 4.17 (1H, br m), 3.62-3.84 (2H, m), 2.92-3.06 (2H, m), 2.88 (1H, dd), 2.78 (1H, br d), 2.65 (1H, app td), 1.42 (9H, s), 1.20 (3H, d).

### Example 230

### (2R)-4-[(1R)-1-(6-Bromo-3-pyridinyl)-2-hydroxyethyl]-2-methyl-1-piperazinecarboxylic Acid, 1,1-Dimethylethyl Ester (321) and (2R)-4-[(1S)-1-(6-Bromo-3-pyridinyl)-2-hydroxyethyl]-2-methyl-1-piperazinecarboxylic Acid, 1,1-Dimethylethyl Ester (322)

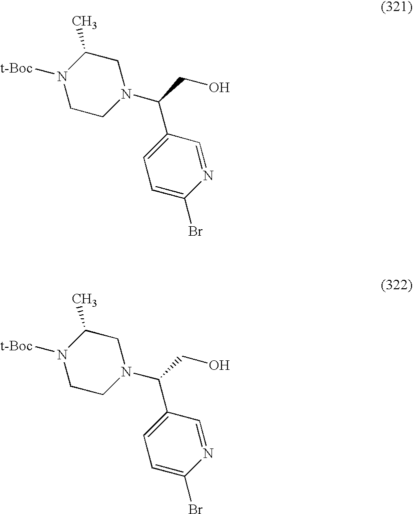

Piperazine 320 (50 mg, 0.25 mmol), glycolaldehyde dimer (17 mg, 0.14 mmol) and 6-bromo-3-pyridinylboronic acid (55 mg, 0.28 mmol) were reacted in accordance with Method V. Purification by column chromatography (silica gel, 0-4% MeOH in DCM) afforded a 1:1 mixture of the title compounds as a viscous, caramel colored oil: LC/MS t_{R} 1.45 min; MS (ES+) m/z 344, 346, 400, 402; ¹H NMR δ_{H} (250 MHz, CDCl₃) 8.24 (1H, d), 7.49 (1H, d), 7.44 (1H, app dt), 4.13-4.33 (1H, br s), 3.71-3.99 (3H, m), 3.56-3.66 (1H, m), 2.83-3.26 (2H, m), 2.62-2.79 (1H, m), 2.40-2.58 (1H, m), 2.00 (0.5H, dd), 1.82 (0.5H, app td), 1.47 and 1.43 (3H, 2 d), 1.43 (9H, s).

### Example 231

### (2R)-4-[(1R)-2-Hydroxy-1-(3-pyridinyl)ethyl]-2-methyl-1-piperazinecarboxylic Acid, 1,1-Dimethylethyl Ester (323) and (2R)-4-[(1S)-2-Hydroxy-1-(3-pyridinyl)ethyl]-2-methyl-1-piperazinecarboxylic Acid, 1,1-Dimethylethyl Ester (324)

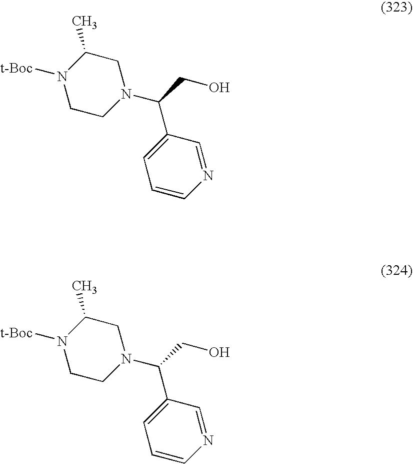

A rapidly stirred suspension of the pyridinyl bromides 321 and 322 (7.6 mg, 0.019 mmol) and 10% palladium on carbon (30% by weight) in EtOH (100 vol) at rt was placed under an atmosphere of H₂ (1 bar) and stirred until the dehalogenation was judged to be complete by LC/MS analysis. The reaction mixture was filtered through Celite® 521 filter agent. The filter cake was washed with EtOH (300 vol) and the combined filtrates concd in vacuo to give the title compounds as a viscous, caramel colored oil: LC/MS t_{R} 1.15 min; MS (ES+) m/z 266, 322.

### Example 232

### (βR,3R)-β-(3-Pyridinyl)-3-methyl-1-piperazineethanol (325) and (βS,3R)-β-(3-Pyridinyl)-3-methyl-1-piperazineethanol (326)

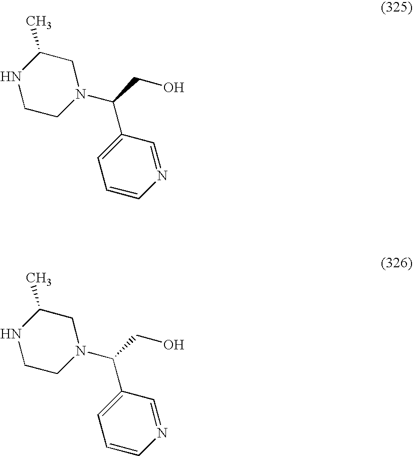

A 1:1 mixture of piperazines 323 and 324 (4.4 mg, 0.014 mmol) was treated with a solution of HCl in 1,4-dioxane (4 M) in accordance with Method X to afford a 1:1 mixture of the bis-HCl salts of title compounds as a vitreous caramel colored solid: LC/MS t_{R} 0.22 min; MS (ES+) m/z 111.6, 222.

### Example 233

### [(2R)-4-[(1R)-2-Hydroxy-1-(3-pyridinyl)ethyl]-2-methyl-1-piperazinyl][5-(4-methoxyphenyl)-6-methyl-7-(trifluoromethyl)pyrazolo[1,5-a]pyrimidin-3-yl]methanone (318) and [(2R)-4-[(1S)-2-Hydroxy-1-(3-pyridinyl)ethyl]-2-methyl-1-piperazinyl][5-(4-methoxyphenyl)-6-methyl-7-(trifluoromethyl)pyrazolo[1,5-a]pyrimidin-3-yl]methanone (319)

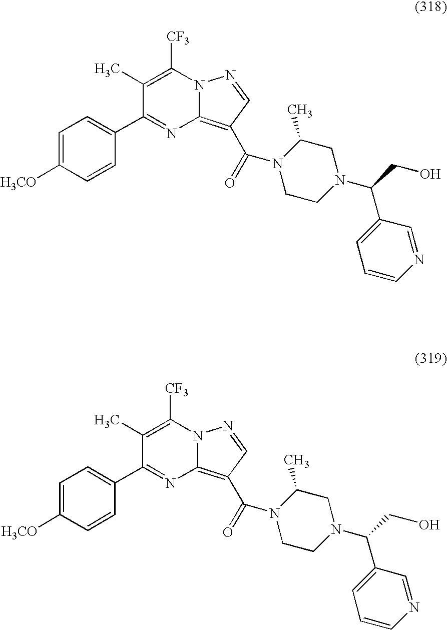

Carboxylic acid 19 (4.8 mg, 0.014 mmol) was treated with HATU (6.3 mg, 0.016 mmol), DIPEA (9.4 μL, 0.055 mmol, d 0.742) and a 1:1 mixture of piperazine HCl salts 325 and 326 (4.1 mg, 0.014 mmol) in accordance with Method G to afford a 1:1 mixture of the title compounds after column chromatography (silica gel, 0-1% MeOH in DCM) as a vitreous yellow solid: LC/MS t_{R} 1.50 min; MS (ES+) m/z 555; ¹H NMR δ_{H} (500 MHz, CDCl₃) 8.59 (1H, d), 8.45-8.49 (1H, m), 8.413 and 8.409 (1H, 2 s), 7.43-7.54 (3H, m), 7.24-7.31 (1H, obs m), 7.06 (2H, d), 3.96 (3H, s), 3.83-5.06 (2H, br m), 3.63-3.79 (2H, m), 3.25-3.57 (1H, br m), 3.10-3.24 (1H, m), 2.54 (3H, q), 2.40-2.99 (3H, br m), 2.15 (0.5H, dd), 2.00 (0.5H, app td), 1.49 and 1.45 (3H, 2 d).

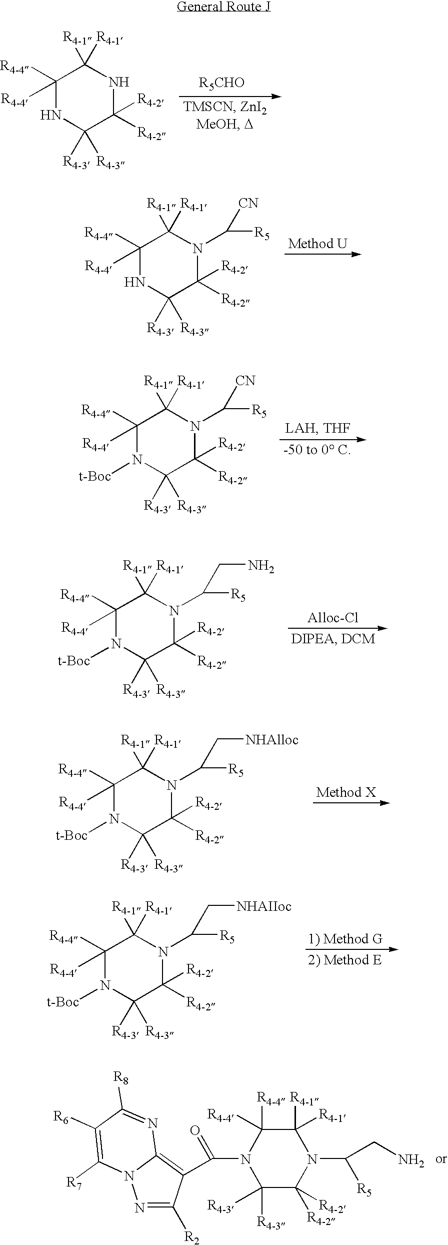

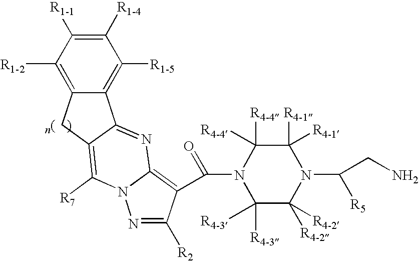

### Description of Methods Used in General Route J

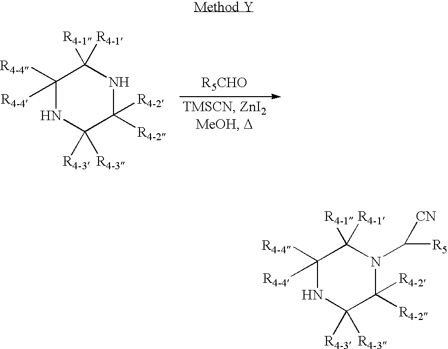

A mixture of the aldehyde (1 equiv) and TMSCN (1.1 equiv) cooled to 0° C. was treated with zinc iodide (0.1 equiv) and stirred at this temperature for 30 min, after which it was treated with a solution of the piperazine (1.5 equiv) in anhydrous MeOH (40 vol). The reaction mixture was heated at reflux for 2 h, allowed to cool to rt and stirred a further 16 h at this temperature. Reaction progress was monitored by LC/MS. On completion the MeOH was removed in vacuo and the residue purified by column chromatography (silica gel, 0-10% MeOH in DCM) to afford the desired product.

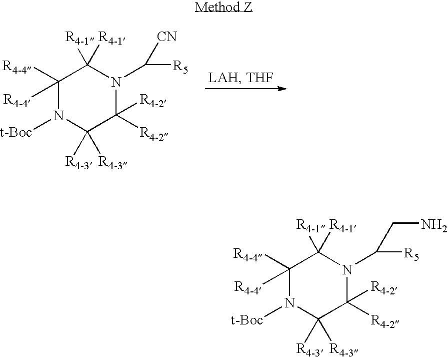

In accordance with the procedure described by Fisher et al. (Bioorg. Med. Chem. Lett., 2005, 15, 4973), a stirred solution of aminonitrile (1 equiv) in THF (20 vol) was cooled to −60° C. and treated with a solution of LAH in THF (1.5 equiv), ensuring the temperature of the reaction mixture did not exceed −50° C. After stirring at −50° C. for a further 30 min, the reaction was allowed to warm to 0° C. over 1 h, at which juncture reaction progress was assessed by LC/MS. If necessary, the reaction was cooled to −60° C. and more LAH (1.5 equiv) was added, monitoring the internal temperature during the addition as detailed above, stirred for a further 30 min at −50° C. then allowed to warm to 0° C. over 1 h. On completion the reaction mixture was quenched at 0° C. via the addition of water (0.26 mL/mL LAH solution), 4 M NaOH (0.26 mL/mL LAH solution) and more water (0.78 mL/mL LAH solution). After stirring the resultant suspension at rt for 1 h, the mixture was dried (Na₂SO₄) and filtered and the filter cake washed with THF (60 vol). The filtrate was evaporated in vacuo and the residue purified by column chromatography (silica gel, 0-5% MeOH in DCM with 1% TEA) to afford the desired product.

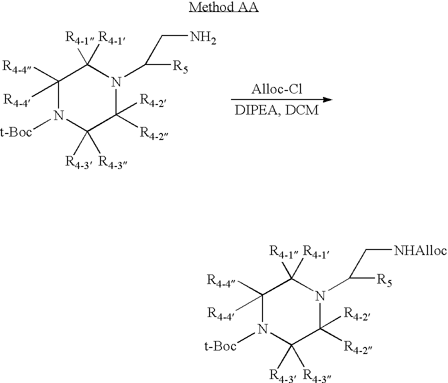

A solution of the amine (1 equiv) and DIPEA (1.5 equiv) in DCM (60 vol) at 0° C. was treated with allyl chloroformate (1 equiv) and allowed to warm to rt and stirred 16 h at this temperature. Reaction progress was monitored by LC/MS. On completion the reaction mixture was diluted with water (60 vol) and extracted into DCM (3×60 vol). The combined DCM phases were dried (Na₂SO₄), filtered and the filtrate reduced in vacuo. Column chromatography (silica gel, 60-80% EtOAc in heptanes) gave the desired product.

### Examples of Compounds Prepared by General Route J

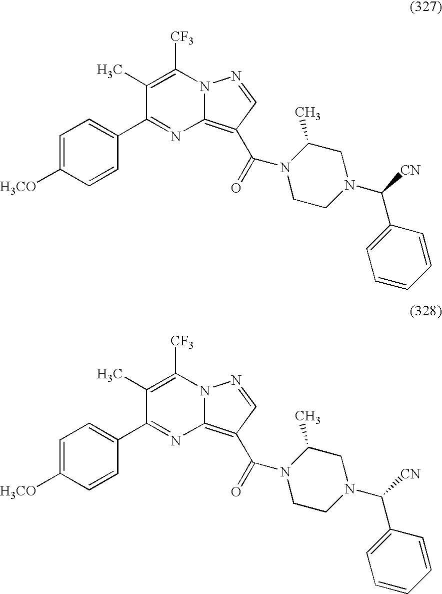

### Example 234

### (αR,3R)-3-Methyl-α-phenyl-1-piperazineacetonitrile (329) and (αSR,3R)-3-Methyl-α-phenyl-1-piperazineacetonitrile (330)

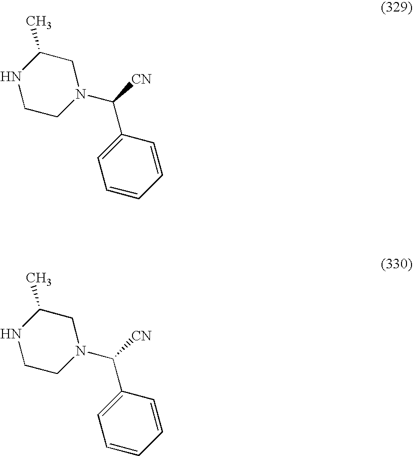

Benzaldehyde (0.96 mL, 9.42 mmol, d 1.045) was treated with TMSCN (1.30 mL, 10.4 mmol, d 0.793), zinc iodide (0.30 g, 0.94 mmol) and (R)-(−)-2-methylpiperazine (1.42 g, 14.1 mmol) in accordance with Method Y. Column chromatography (silica gel, 0-10% MeOH in DCM) afforded a 1:1 mixture of the title diastereomers as a viscous orange oil: LC/MS t_{R} 0.89 min; MS (ES+) m/z 189, 216; ¹H NMR δ_{H} (250 MHz, CDCl₃) 7.47-7.61 (2H, m), 7.32-7.46 (3H, m), 4.84 and 4.82 (1H, 2 s), 2.67-3.14 (4H, m), 2.56 (0.5H, app td), 2.39-2.50 (1H, m), 2.23 (0.5H, app t), 2.15 (0.5H, app td), 1.83 (0.5H, app t), 1.10 and 0.97 (3H, 2 d).

### Example 235

### (αR,3R)-4-[[5-(4-Methoxyphenyl)-6-methyl-7-(trifluoromethyl)pyrazolo[1,5-a]pyrimidin-3-yl]carbonyl]-3-methyl-α-phenyl-1-piperazineacetonitrile (327) and (αS,3R)-4-[[5-(4-Methoxyphenyl)-6-methyl-7-(trifluoromethyl)pyrazolo[1,5-a]pyrimidin-3-yl]carbonyl]-3-methyl-α-phenyl-1-piperazineacetonitrile (328)

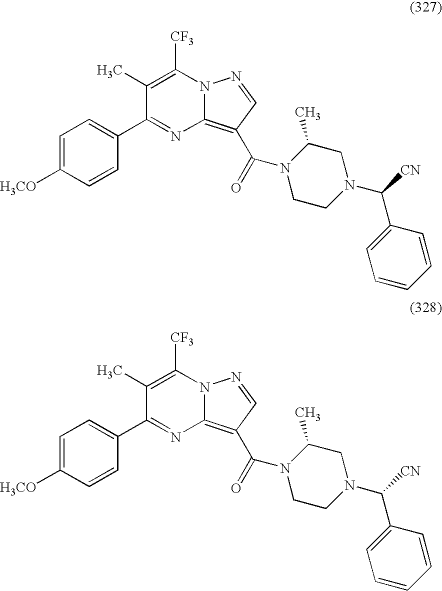

Carboxylic acid 19 (30 mg, 0.085 mmol) was treated with HATU (39 mg, 0.10 mmol), DIPEA (20.7 μL, 0.12 mmol, d 0.742) and a 1:1 mixture of piperazines 329 and 330 (19.3 mg, 0.09 mmol) in accordance with Method G. Dilution of the reaction mixture with water (5 mL) and isolation of the resultant precipitate via vacuum filtration afforded a 1:1 mixture of the title compounds as a yellow powder: LC/MS t_{R} 2.38 min; MS (ES+) m/z 522, 549, 571; ¹H NMR δ_{H} (250 MHz, CDCl₃) 8.47 (1H, s), 7.35-7.62 (7H, m), 7.08 (2H, d), 4.91 and 4.81 (1H, 2 s), 3.98-4.77 (1H, br m), 3.92 (3H, s), 3.47 and 3.21 (1H, 2 br s), 2.65-3.03 (2H, m), 2.58 (3H, q), 2.01-2.64 (3H, m), 1.43 and 1.24 (3H, 2 d).

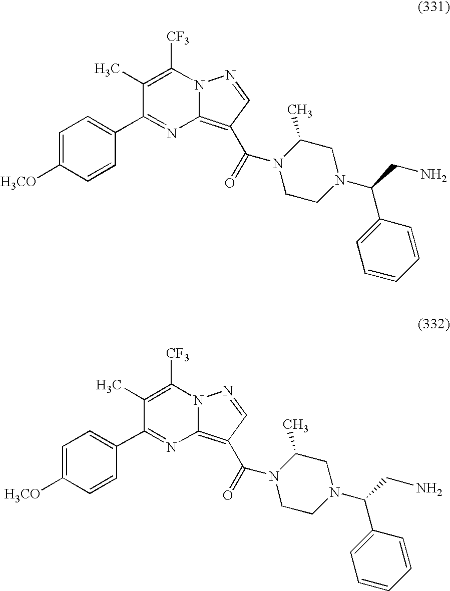

### Example 236

### (2R)-4-[(R)-Cyanophenylmethyl]-2-methyl-1-piperazinecarboxylic Acid, 1,1-Dimethylethyl Ester (333) and (2R)-4-[(S)-Cyanophenylmethyl]-2-methyl-1-piperazinecarboxylic Acid, 1,1-Dimethylethyl Ester (334)

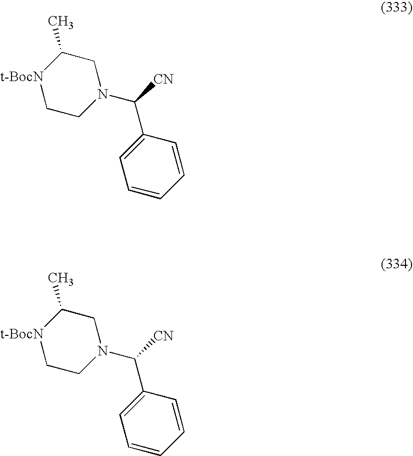

A 1:1 mixture of piperazines 329 and 330 (1.0 g, 4.64 mmol) was treated with di-tert-butyl dicarbonate (1.06 g, 4.87 mmol) employing the procedure of Method U to afford a 1:1 mixture of the title compounds admixed with di-tert-butyl dicarbonate as a viscous, orange oil: LC/MS t_{R} 2.32 min; MS (ES+) m/z 189, 233, 260, 301; ¹H NMR δ_{H} (250 MHz, CDCl₃) 7.48-7.59 (2H, m), 7.32-7.47 (3H, m), 4.91 and 4.81 (1H, 2 s), 4.36 and 4.20 (1H, 2 br s), 3.96 and 3.80 (1H, 2 d), 3.22 (0.5H, 2 app td), and 2.97 (0.5H, app td), 2.86 (0.5H, br app dt), 2.78 (0.5H, dd), 2.65 (0.5H, app dt), 2.58 (0.5H, app dd), 2.46 (m, 0.5H), 2.33 (0.5H, app dd), 2.29 (0.5H, app dt), 2.16 (0.5H, app dt), 1.45 (9H, s), 1.31 and 1.10 (3H, 2 d).

### Example 237

### (2R)-4-[(1R)-2-Amino-1-phenylethyl]-2-methyl-1-piperazinecarboxylic Acid, 1,1-Dimethylethyl Ester (335) and (2R)-4-[(1R)-2-Amino-1-phenylethyl]-2-methyl-1-piperazinecarboxylic Acid, 1,1-Dimethylethyl Ester (336)

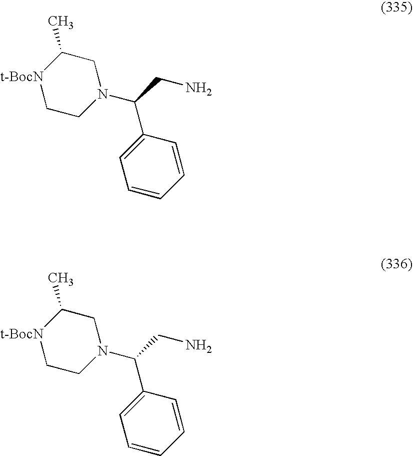

A 1:1 mixture of aminonitriles 333 and 334 (0.93 g, 2.96 mmol) was treated with two aliquots of LAH (1.30 mL, 4.44 mmol, 3.5 M solution in PhMe/THF) in accordance with Method Z. Column chromatography (silica gel, 0-5% MeOH in DCM with 1% TEA) afforded a 1:1 mixture of the title diastereomers as a viscous colorless oil: LC/MS t_{R} 1.41 min; MS (ES+) m/z 264, 320; ¹H NMR δ_{H} (250 MHz, CDCl₃) 7.12-7.38 (5H, m), 4.00-4.28 (2H, br s), 3.66-3.89 (2H, m), 2.83-2.97 (4H, m), 2.56-2.79 (4H, m), 1.45 (9H, s), 1.20 (3H, d).

### Example 238

### (2R)-2-Methyl-4-[(1R)-1-phenyl-2-[[(2-propen-1-yloxy)carbonyl]amino]ethyl]-1-piperazinecarboxylic Acid, 1,1-Dimethylethyl Ester (337) and (2R)-2-Methyl-4-[(1S)-1-phenyl-2-[[(2-propen-1-yloxy)carbonyl]amino]ethyl]-1-piperazinecarboxylic Acid, 1,1-Dimethylethyl Ester (338)

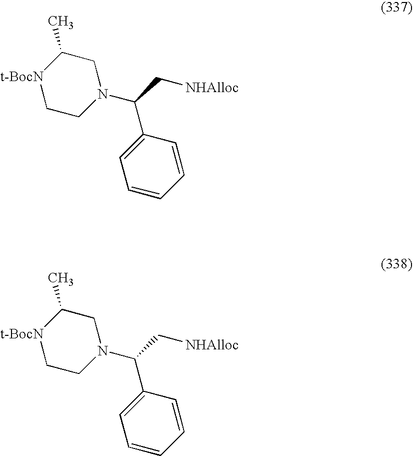

A 1:1 mixture of amines 335 and 336 (0.37 g, 0.92 mmol) was treated with DIPEA (0.26 mL, 1.49 mmol, d 0.742) and allyl chloroformate (0.10 mL, 0.92 mmol, d 1.136) in accordance with Method AA. Column chromatography (silica gel, 60-80% EtOAc in heptanes) afforded a 1:1 mixture of the title diastereomers as a pale yellow oil: LC/MS t_{R} 1.66 and 1.71 min; MS (ES+) m/z 404; ¹H NMR δ_{H} (250 MHz, CDCl₃) 7.25-7.41 (3H, m), 7.13-7.25 (2H, m), 5.77-6.01 (1H, m), 5.15-5.36 (2H, m), 5.02 (1H, br s), 4.61 and 4.55 (1H, 2 obs br s), 4.55 (1H, d), 4.22 and 4.12 (1H, 2 br s), 3.59-3.89 (2H, m), 3.37-3.56 (2H, m), 3.13 (0.5H, app td), 2.99 (0.5H, app td), 2.87 (0.5H, br d), 2.75 (1H, dd), 2.52 (0.5H, d), 2.33 (0.5H, dd), 2.14 (0.5H, app td), 1.93 (0.5H, dd), 1.75 (0.5H app td), 1.42 (9H, s), 1.28 and 1.20 (3H, 2 d).

### Example 239

### N-[(2R)-2-[(3R)-3-Methyl-1-piperazinyl]-2-phenylethyl]carbamic Acid, 2-Propen-1-yl Ester (339) and N-[(2S)-2-[(3R)-3-Methyl-1-piperazinyl]-2-phenylethyl]carbamic Acid, 2-Propen-1-yl Ester (340)

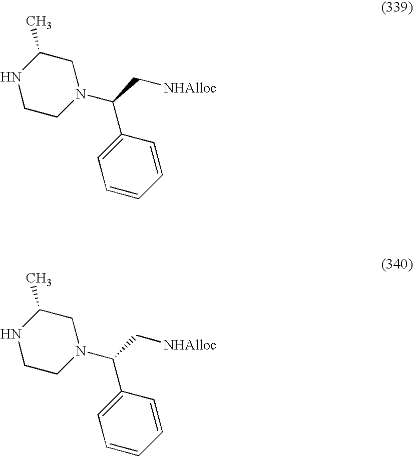

A solution of a 1:1 mixture of tert-butyl carbamates 337 and 338 (0.20 g, 0.48 mmol) in 1,4-dioxane (2 mL) was treated with a solution of HCl in 1,4-dioxane (12 mL, 4 M) in accordance with Method X. A 1:1 mixture of the HCl salts of the title compounds was isolated as an orange semi-solid: LC/MS t_{R} 1.13 min; MS (ES+) m/z 304; ¹H NMR δ_{H} (250 MHz, CD₃OD) 7.60-7.70 (2H, m), 7.49-7.59 (3H, m), 5.75-6.12 (1H, m), 5.09-5.40 (2H, m), 4.47-4.76 (3H, m), 3.98-4.29 (2H, m), 3.47-3.96 (5H, m), 2.99-3.42 (3H, obs m), 1.44 and 1.35 (3H, 2 d).

### Example 240

### N-[(2R)-2-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]-2-phenylethyl]carbamic Acid, 2-Propen-1-yl Ester (341) and N-[(2S)-2-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]-2-phenylethyl]carbamic Acid, 2-Propen-1-yl Ester (342)

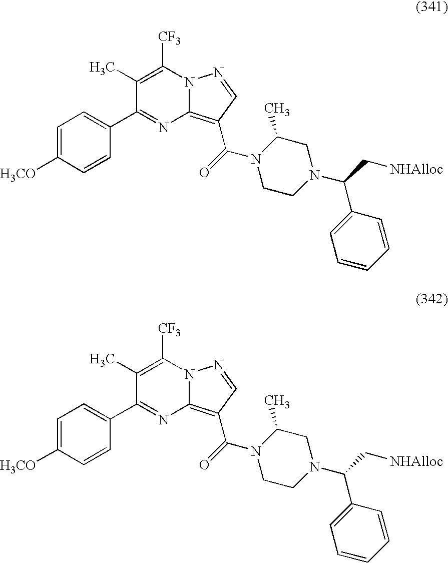

Carboxylic acid 19 (172 mg, 0.49 mmol) was treated with HATU (224 mg, 0.59 mmol), DIPEA (0.26 mL, 1.47 mmol, d 0.742) and a 1:1 mixture of the HCl salts of piperazines 339 and 340 (175 mg, 0.52 mmol) in accordance with Method G to afford a 1:1 mixture of the title compounds after column chromatography (silica gel, 40-70% EtOAc in heptanes) as a vitreous yellow solid: LC/MS t_{R} 1.99 and 2.04 min; MS (ES+) m/z 637, 659; ¹H NMR δ_{H} (360 MHz, CDCl₃) 8.40 (1H, s), 7.47 (2H, d), 7.30-7.38 (3H, m), 7.11-7.21 (2H, m), 7.03 (2H, d), 5.81-5.98 (1H, m), 5.17-5.32 (2H, m), 4.98 (1H, br s), 4.55 (2H, d), 4.04-4.45 (1H, br s), 3.94 (3H, s), 3.59-3.78 (1H, m), 3.40-3.57 (2H, m), 3.26 (1H, br s), 2.66-2.98 (2H, m), 2.53 (3H, q), 2.25-2.62 (2H, m), 2.10 (0.5H, dd), 1.95 (0.5H, app td), 1.42 and 1.35 (3H, 2 d).

### Example 241

### [(2R)-4-[(1R)-2-Amino-1-phenylethyl]-2-methyl-1-piperazinyl][5-(4-methoxyphenyl)-6-methyl-7-(trifluoromethyl)pyrazolo[1,5-a]pyrimidin-3-yl]methanone (331) and [(2R)-4-[(1S)-2-Amino-1-phenylethyl]-2-methyl-1-piperazinyl][5-(4-methoxyphenyl)-6-methyl-7-(trifluoromethyl)pyrazolo[1,5-a]pyrimidin-3-yl]methanone (332)

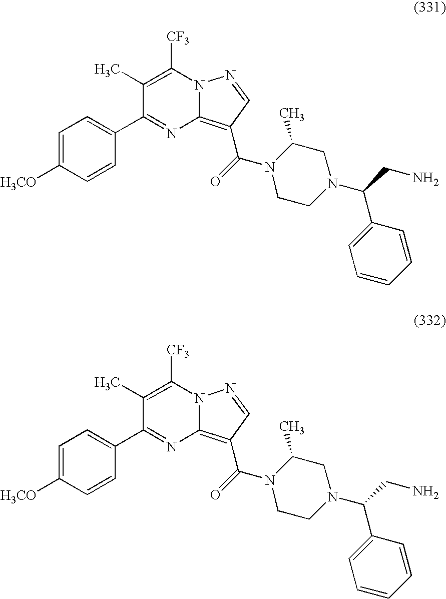

A 1:1 mixture of allyl carbamates 341 and 342 (234 mg, 0.37 mmol) was treated with 1,3-dimethylbarbituric acid (58 mg, 0.37 mmol) and Pd(PPh₃)₄ (43 mg, 0.037 mmol) employing the procedure of Method E to give the title compound after column chromatography (silica gel, 0-4% MeOH in DCM with 1% TEA; repeated once using the same conditions) as a light brown powder. Reverse phase preparative HPLC (5-95% MeCN in water) on 30 mg of this material afforded an analytically pure sample as a vitreous yellow solid: LC/MS t_{R} 1.59 min; MS (ES+) m/z 277, 553; ¹H NMR δ_{H} (250 MHz, CDCl₃) 8.40 (1H, s), 7.48 (2H, d), 7.27-7.40 (3H, m), 7.15-7.24 (2H, m), 7.03 (2H, d), 3.58-5.05 (2H, br m), 3.93 (3H, s), 3.22-3.56 (2H, m), 2.64-3.19 (3H, m), 2.53 (3H, q), 2.20-2.59 (2H, m), 2.07 (0.5H, dd), 1.93 (0.5H, app td), 1.44 and 1.36 (3H, 2 d).

### Example 242

### N-[(2R)-2-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]-2-phenylethyl]acetamide (343) and N-[(2S)-2-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]-2-phenylethyl]acetamide (344)

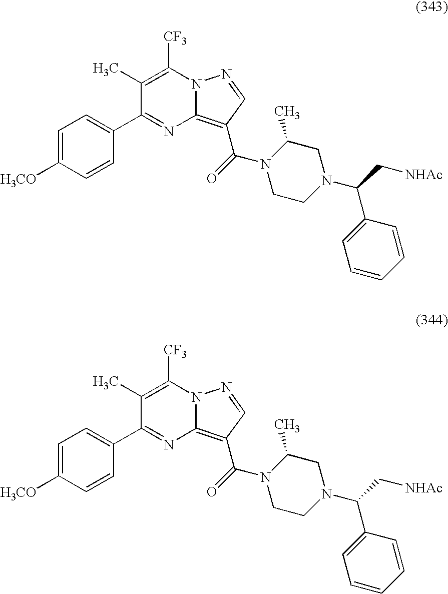

A 1:1 mixture of amines 331 and 332 (39 mg, 0.071 mmol, ca. 60% purity) and TEA (20 μL, 0.14 mmol, d 0.726) in DCM (2 mL) was cooled to 0° C. and treated with acetyl chloride (5.0 μL, 0.071 mmol, d 1.104). After stirring at rt for 1 h, the reaction mixture was diluted with DCM (10 mL) and satd NaHCO₃ (10 mL) and the two phases separated. The organic phase was washed with water (2×10 mL) and brine (10 mL), dried (Na₂SO₄), filtered and reduced in vacuo. Reverse phase preparative HPLC (5-95% MeCN in water) afforded the title compound as a vitreous yellow solid: LC/MS t_{R} 1.68 min; MS (ES+) m/z 595, 617; ¹H NMR δ_{H} (250 MHz, CDCl₃) 8.40 (1H, s), 7.47 (2H, d), 7.32 (3H, app br s), 7.16 (2H, m), 7.03 (2H, d), 5.74 (1H, br s), 4.04-4.85 (1H, br m), 3.94 (3H, s), 3.10-3.88 (5H, m), 2.53 (3H, q), 2.25-2.97 (3H, m), 2.12 (0.5H, dd), 1.96 and 1.95 (3H, 2 s), 1.92 (0.5H, obs app td), 1.42 and 1.34 (3H, 2 d).

### Example 243

### N-[(2R)-2-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]-2-phenylethyl]methanesulfonamide (345) and N-[(2S)-2-[(3R)-4-[[5-(4-Methoxyphenyl)-6-methyl-7-(trifluoromethyl)pyrazolo[1,5-a]pyrimidin-3-yl]carbonyl]-3-methyl-1-piperazinyl]-2-phenylethyl]methanesulfonamide (346)

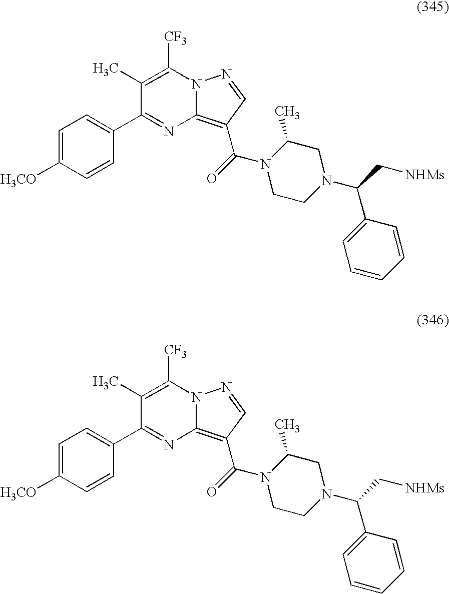

A 1:1 mixture of amines 331 and 332 (25 mg, 0.045 mmol, ca. 60% purity) and TEA (6.3 μL, 0.045 mmol, d 0.726) in DCM (2 mL) was cooled to 0° C. and treated with MsCl (1.9 μL, 0.025 mmol, d 1.48). After stirring at rt for 1 h, the reaction mixture was diluted with DCM (10 mL) and satd NaHCO₃ (10 mL) and the two phases separated. The organic phase was washed with water (2×10 mL) and brine (10 mL), dried (Na₂SO₄), filtered and reduced in vacuo. Reverse phase preparative HPLC (0.1% TFA, 5-95% MeCN in water) afforded the TFA salt of the title compound as a vitreous yellow solid: LC/MS t_{R} 1.84 and 1.91 min; MS (ES+) m/z 631, 653; ¹H NMR δ_{H} (250 MHz, CDCl₃) 8.49 and 8.48 (1H, 2 s), 7.44 (2H, d), 7.36 (3H, app br s), 7.16 (2H, obs m), 7.14 (2H, d), 3.99-5.04 (3H, br m), 3.95 (3H, s), 3.10-3.96 (5H, m), 2.98 and 2.92 (3H, 2 s), 2.85 (1H, br s), 2.65 (1H, br s), 2.53 (3H, q), 2.40-2.56 (1H, obs m), 1.59 and 1.53 (3H, 2 d).

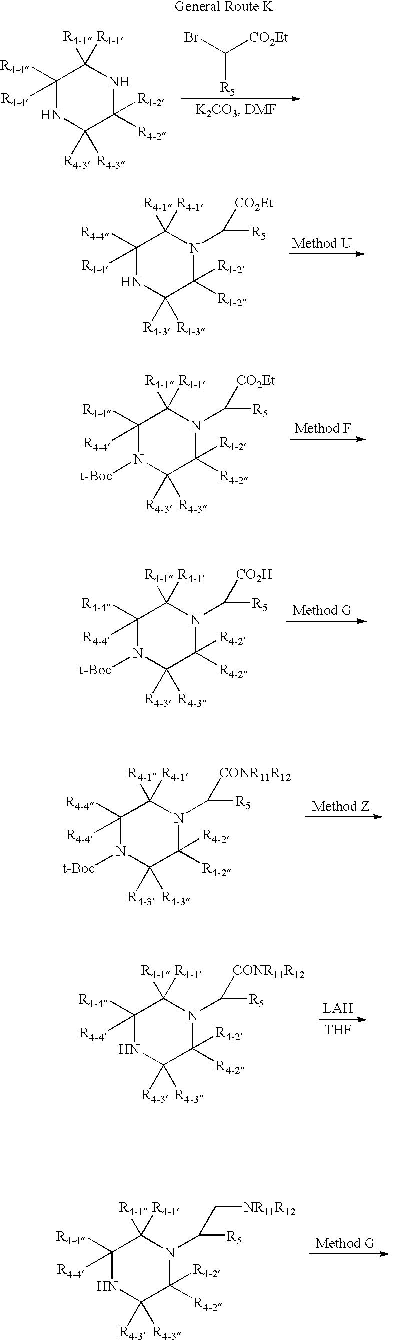

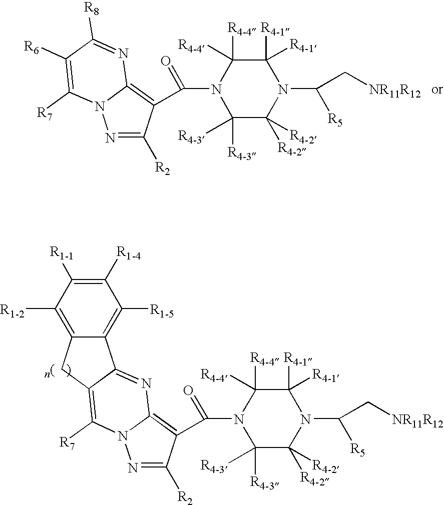

### Description of Methods Used in General Route K

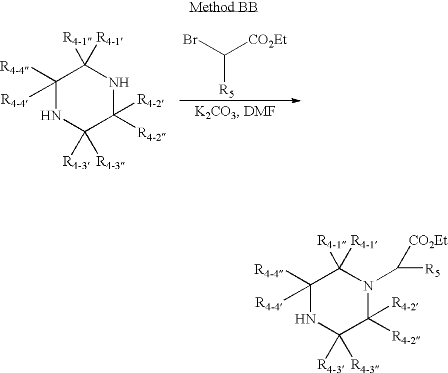

The α-bromoacetate (1 equiv) was added dropwise over 5-10 min to a stirred rt suspension of the piperazine (1.5 equiv) and K₂CO₃ (3 equiv) in DMF (15 vol) and stirred at rt for 16 h. Reaction progress was monitored by LC/MS. On completion the reaction mixture was diluted with water (50 vol) and extracted into MTBE (2×50 vol). The combined MTBE phases were washed with water (2×50 vol) and brine (50 vol), dried (Na₂SO₄) and filtered and the filtrate reduced in vacuo to give the desired product.

### Examples of Compounds Prepared by General Route K

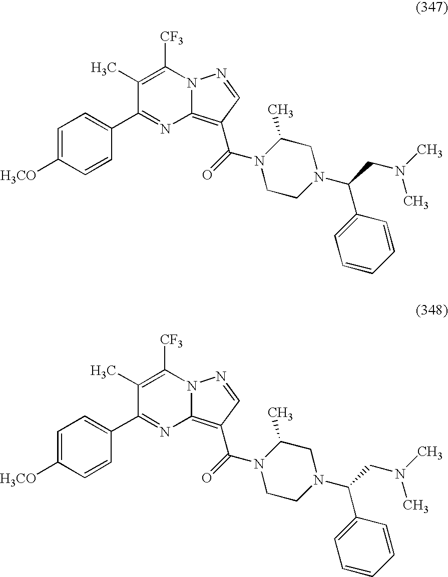

### Example 244

### (αR,3R)-3-Methyl-α-phenyl-1-piperazineacetic Acid, Ethyl Ester (349) and (αS,3R)-3-Methyl-α-phenyl-1-piperazineacetic Acid, Ethyl Ester (350)

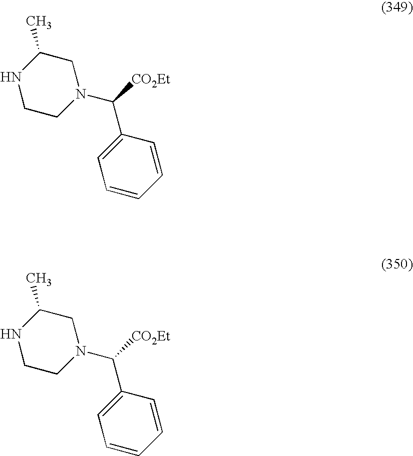

(R)-(−)-2-Methylpiperazine (1.24 g, 12.3 mmol) was treated with K₂CO₃ (3.41 g, 24.7 mmol) and ethyl α-bromophenylacetate (1.44 mL, 8.22 mmol, d 1.389) in accordance with Method BB to afford a 1:1 mixture of the title diastereomers as a viscous, colorless oil: LC/MS t_{R} 1.08 min; MS (ES+) m/z 263; ¹H NMR δ_{H} (360 MHz, CDCl₃) 7.39-7.46 (2H, m), 7.28-7.36 (3H, m), 4.05-4.24 (2H, m), 3.954 and 3.947 (1H, 2 s), 2.82-3.06 (4H, m), 2.56-2.63 (1H, m), 2.18 (0.5H, app td), 1.89 (0.5H, app td), 1.83 (0.5H, dd), 1.55 (0.5H, dd), 1.19 (3H, t), 1.02 and 0.92 (3H, 2 d).

### Example 245

### (αR,3R)-4-[(1,1-Dimethylethoxy)carbonyl]-3-methyl-α-phenyl-1-piperazineacetic Acid, Ethyl Ester (351) and (αS,3R)-4-[(1,1-Dimethylethoxy)carbonyl]-3-methyl-α-phenyl-1-piperazineacetic Acid, Ethyl Ester (352)

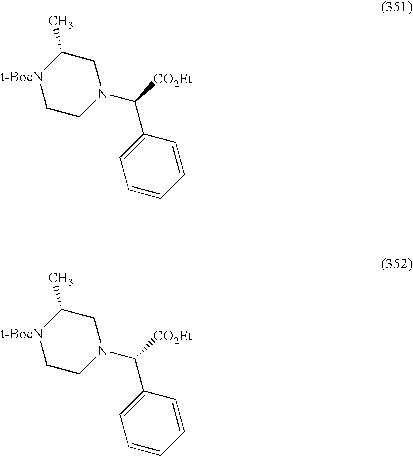

A 1:1 mixture of piperazines 349 and 350 (1.0 g, 3.81 mmol) was treated with di-tert-butyl dicarbonate (0.88 g, 4.00 mmol) employing the procedure of Method U to afford a 1:1 mixture of the title compounds as a colorless oil: LC/MS t_{R} 2.32 min; MS (ES+) m/z 363, 385; ¹H NMR δ_{H} (250 MHz, CDCl₃) 7.39-7.49 (2H, m), 7.28-7.38 (3H, m), 4.05-4.28 (3H, m), 4.00 and 3.96 (1H, 2 s), 3.82 and 3.74 (1H, 2 br d), 3.18 (0.5H, app td), 3.07 (0.5H, app td), 2.88 (0.5H, br d), 2.68 (0.5H, app dt), 2.62 (0.5H, br d), 2.50 (0.5H, app dt), 2.39 (0.5H, dd), 2.19 (0.5H, app td), 2.07 (0.5H, dd), 1.95 (0.5H, app td), 1.44 (9H, s), 1.29 and 1.23 (3H, 2 d), 1.22 and 1.21 (3H, 2 t).

### Example 246

### (αR,3R)-4-[(1,1-Dimethylethoxy)carbonyl]-3-methyl-α-phenyl-1-piperazineacetic Acid (353) and (αS,3R)-4-[(1,1-Dimethylethoxy)carbonyl]-3-methyl-α-phenyl-1-piperazineacetic Acid (354)

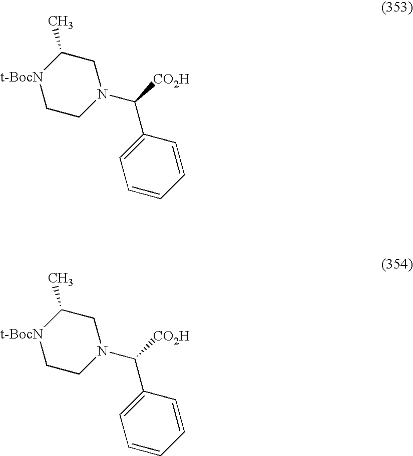

A 1:1 mixture of ethyl esters 351 and 352 (0.80 g, 2.21 mmol) was treated with aqueous NaOH in accordance with Method F. On completion of the reaction, the EtOH was removed in vacuo and the aqueous residue remaining lowered to pH 5-6 by the addition of solid citric acid. The aqueous phase was extracted with EtOAc (3×25 mL), the combined EtOAc phases washed with brine (25 mL), dried (Na₂SO₄) and filtered and the filtrate reduced in vacuo to give a 1:1 mixture of the title compounds as a vitreous colorless solid: LC/MS t_{R} 1.36 and 1.41 min; MS (ES+) m/z 335.

### Example 247

### (2R)-4-[(1R)-2-(Dimethylamino)-2-oxo-1-phenylethyl]-2-methyl-1-piperazinecarboxylic Acid, 1,1-Dimethylethyl Ester (355) and (2R)-4-[(1S)-2-(Dimethylamino)-2-oxo-1-phenylethyl]-2-methyl-1-piperazinecarboxylic Acid, 1,1-Dimethylethyl Ester (356)

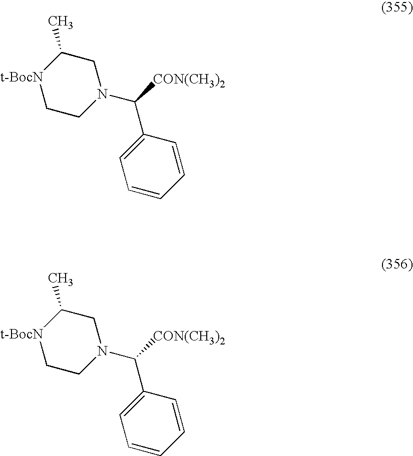

A 1:1 mixture of carboxylic acids 353 and 354 (150 mg, 0.45 mmol) was treated with HATU (205 mg, 0.54 mmol), DIPEA (0.24 mL, 1.35 mmol, d 0.742) and dimethylamine hydrochloride (55 mg, 0.68 mmol) in accordance with Method G to afford a 1:1 mixture of the title compounds as a viscous, pale brown oil: LC/MS t_{R} 1.31 and 1.40 min; MS (ES+) m/z 362, 384; ¹H NMR δ_{H} (250 MHz, CDCl₃) 7.28-7.45 (5H, m), 4.36 and 4.32 (1H, 2 s), 4.15 (1H, br s), 3.76 (1H, m), 3.06-3.21 (1H, m), 3.03 and 2.99 (3H, 2 s), 2.93 (3H, s), 2.84 (0.5H, br d), 2.75 (0.5H, br d), 2.60-2.69 (1H, m), 2.53 (0.5H, dd), 2.28 (0.5H, app td), 2.13 (0.5H, dd), 2.08 (0.5H, app td), 1.43 (9H, s), 1.26 and 1.23 (3H, 2 d).

### Example 248

### (αR,3R)—N,N,3-Trimethyl-α-phenyl-1-piperazineacetamide (357) and (αS,3R)—N,N,3-Trimethyl-α-phenyl-1-piperazineacetamide (358)

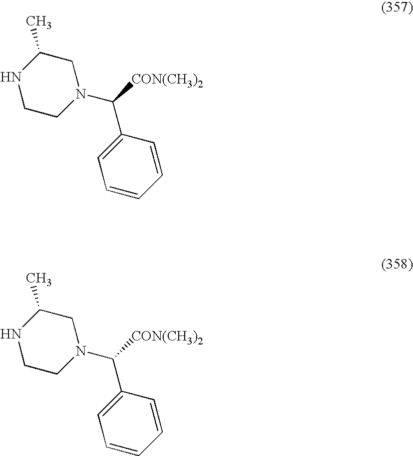

The 1:1 mixture of tert-butyl carbamates 355 and 356 (166 mg, 0.46 mmol) was treated with 4 M HCl in 1,4-dioxane in accordance with Method X. Column chromatography (SCX silica gel, eluting with MeOH then NH₃ in MeOH, 2 M) afforded a 1:1 mixture of the title compounds as a colorless oil: LC/MS t_{R} 0.83 min; MS (ES+) m/z 262; ¹H NMR δ_{H} (250 MHz, CDCl₃) 7.28-7.46 (5H, m), 4.25 and 4.21 (1H, s), 3.00 and 2.99 (3H, 2 s), 2.94 and 2.93 (3H, 2 s), 2.80-3.07 (4H, m), 2.63-2.77 (1H, m), 2.24 (0.5H, app td), 1.92 (0.5H, app td), 1.86 (0.5H, dd), 1.75 (1H, br s), 1.60 (0.5H, dd), 1.01 and 0.93 (3H, 2 d).

### Example 249

### (βR,3R)—N,N,3-Trimethyl-β-phenyl-1-piperazineethanamine (359) and (βS,3R)—N,N,3-Trimethyl-β-phenyl-1-piperazineethanamine (360)

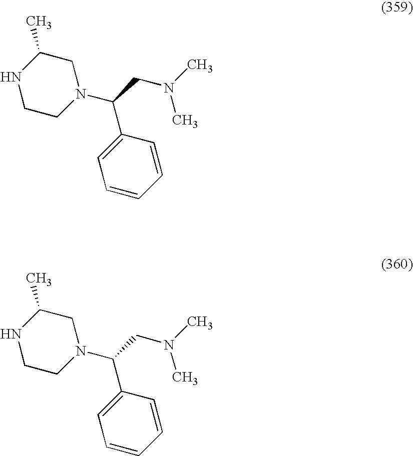

A solution of a 1:1 mixture of amides 357 and 358 (106 mg, 0.41 mmol) in THF (2.5 mL) at 0° C. was treated with LAH (0.23 mL, 0.81 mmol, 3.5 M in PhMe/THF) and stirred at rt for 2 h. Reaction progress was monitored by LC/MS. On completion the reaction mixture was quenched via the addition of water (31 μL), 4 M NaOH (31 μL), and more water (100 μL). On stirring the resultant suspension at rt for 1 h, the mixture was dried (Na₂SO₄), filtered, and the filter cake washed with THF (25 mL). The combined filtrates were evaporated in vacuo and the residue purified by column chromatography (silica gel, 0-20% MeOH in DCM with 2% TEA) to afford a 1:1 mixture of the title compounds as a colorless oil: LC/MS t_{R} 0.29 min; MS (ES+) m/z 248; ¹H NMR δ_{H} (250 MHz, CD₃OD) 7.19-7.45 (5H, m), 3.78 (1H, app td), 3.12 (0.5H, dd), 3.07 (0.5H, dd), 2.72-3.00 (6H, m), 2.35 (6H, s), 2.07-2.49 (1H, br m), 1.89 (1H, s), 1.87 (0.5H, obs dd), 1.63 (0.5H, dd), 1.05 and 1.03 (3H, 2 d).

### Example 250

### [(2R)-4-[(1R)-2-(Dimethylamino)-1-phenylethyl]-2-methyl-1-piperazinyl][5-(4-methoxyphenyl)-6-methyl-7-(trifluoromethyl)pyrazolo[1,5-a]pyrimidin-3-yl]methanone (347) and [(2R)-4-[(1S)-2-(Dimethylamino)-1-phenylethyl]-2-methyl-1-piperazinyl][5-(4-methoxyphenyl)-6-methyl-7-(trifluoromethyl)pyrazolo[1,5-a]pyrimidin-3-yl]methanone (348)

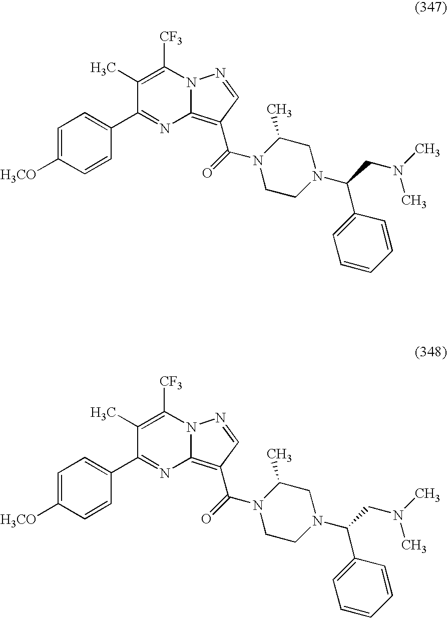

Carboxylic acid 19 (39 mg, 0.11 mmol) was treated with HATU (51 mg, 0.13 mmol), DIPEA (27 μL, 0.16 mmol, d 0.742) and a 1:1 mixture of piperazines 359 and 360 (33 mg, 0.13 mmol) in accordance with Method G to afford a 1:1 mixture of the title compounds after column chromatography (silica gel, 0-1% MeOH in EtOAc) as a vitreous yellow solid: LC/MS t_{R} 1.59 min; MS (ES+) m/z 581; ¹H NMR δ_{H} (250 MHz, CDCl₃) 8.39 and 8.38 (1H, 2 s), 7.48 (2H, d), 7.15-7.36 (5H, m), 7.02 (2H, d), 4.02-4.86 (1H, br s), 3.93 (3H, s), 3.54 (1H, m), 3.35 (1H, br s), 2.53 (3H, q), 2.32-3.09 (5H, m), 2.25 and 2.22 (6H, 2 s), 2.05-2.22 (2H, m), 1.38 and 1.33 (3H, 2 d).

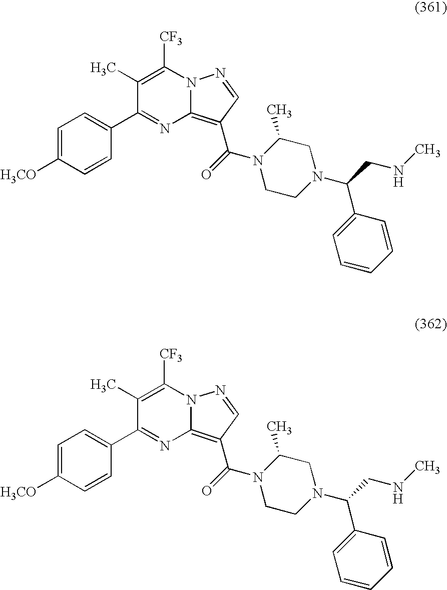

### Example 251

### (2R)-4-[(1R)-2-(Methyl-2-propen-1-ylamino)-2-oxo-1-phenylethyl]-2-methyl-1-piperazinecarboxylic Acid, 1,1-Dimethylethyl Ester (363) and (2R)-4-[(1S)-2-(Methyl-2-propen-1-ylamino)-2-oxo-1-phenylethyl]-2-methyl-1-piperazinecarboxylic Acid, 1,1-Dimethylethyl Ester (364)

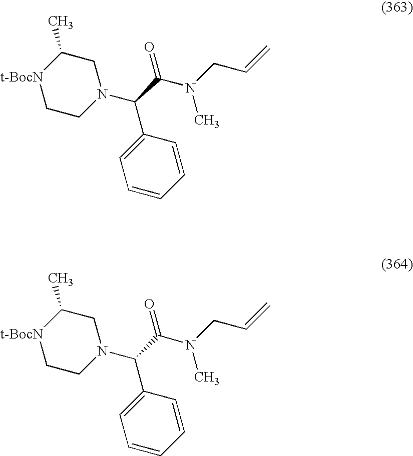

A 1:1 mixture of carboxylic acids 353 and 354 (300 mg, 0.90 mmol) was treated with HATU (410 mg, 1.08 mmol), DIPEA (0.22 mL, 1.26 mmol, d 0.742) and N-methyl allylamine (91 μL, 0.95 mmol, d 0.741) in accordance with Method G to afford a 1:1 mixture of the title compounds as a viscous, orange oil: LC/MS t_{R} 1.46 and 1.59 min; MS (ES+) m/z 332, 388, 410; ¹H NMR δ_{H} (360 MHz, CDCl₃) 7.38-7.44 (2H, m), 7.28-7.38 (3H, m), 5.50-5.77 (1H, m), 5.17 (0.5H, dd), 5.11 (0.5H, dd), 5.10 (0.5H, d), 5.02 (0.5H, d), 4.36 (0.5H, d), 4.26 (0.5H, d), 3.65-4.20 (4H, m), 3.10 (1H, m), 2.87-2.97 (4H, m), 2.48-2.78 (2H, m), 2.31 (0.5H, app td), 2.16 (0.5H, dd), 1.43 (9H, s), 1.25 and 1.23 (3H, 2 d).

### Example 252

### (αR,3R)—N,3-Dimethyl-α-phenyl-N-(2-propen-1-yl)-1-piperazineacetamide (365) and (αS,3R)—N,3-Dimethyl-α-phenyl-N-(2-propen-1-yl)-1-piperazineacetamide (366)

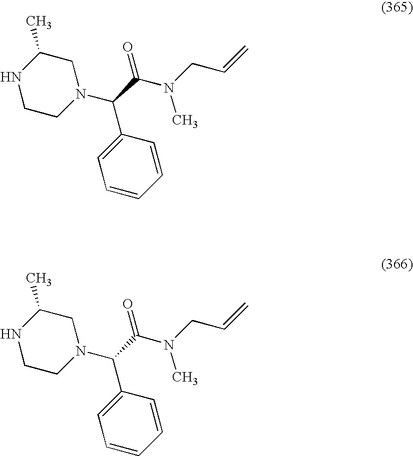

The 1:1 mixture of tert-butyl carbamates 363 and 364 (324 mg, 0.84 mmol) was treated with 4 M HCl in 1,4-dioxane in accordance with Method X. On completion, the reaction solvent was evaporated and the residue obtained treated with satd NaHCO₃ solution (25 mL). The aqueous phase was extracted with EtOAc (3×25 mL), the combined EtOAc phases dried (Na₂SO₄) and filtered and the filtrate reduced in vacuo to afford a 1:1 mixture of the title compounds as a caramel colored oil: LC/MS t_{R} 1.08 min; MS (ES+) m/z 288; ¹H NMR δ_{H} (250 MHz, CDCl₃) 7.28-7.45 (5H, m), 5.46-5.80 (1H, m), 4.93-5.22 (2H, m), 4.26 (0.5H, d), 4.16 (0.5H, d), 4.06 (0.5H, br d), 3.98 (1H, d), 3.57-3.83 (1.5H, m), 2.90 (3H, s), 2.84-3.05 (3H, obs m), 2.70 (1H, m), 2.18-2.36 (0.5H, m), 2.09 (1H, br s), 1.82-2.03 (1H, m), 1.64 (0.5H, dd), 1.02 and 0.95 (3H, 2 dd).

### Example 253

### (βR,3R)—N,3-Dimethyl-β-phenyl-(2-propen-1-yl)-1-piperazineethanamine (367) and (βS,3R)—N,3-Dimethyl-β-phenyl-(2-propen-1-yl)-1-piperazineethanamine (368)

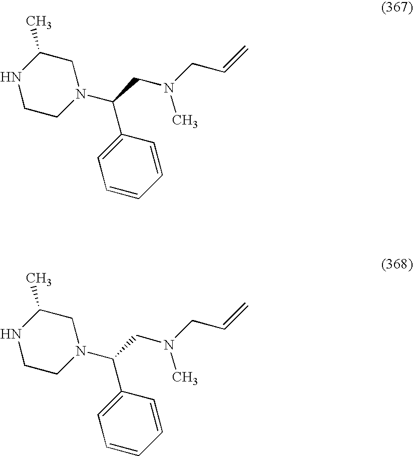

A solution of a 1:1 mixture of amides 365 and 366 (227 mg, 0.79 mmol) in THF (5 mL) at 0° C. was treated with LAH (0.45 mL, 1.58 mmol, 3.5 M in PhMe/THF) stirred at rt for 2 h. Reaction progress was monitored by LC/MS. On completion the reaction mixture was quenched via the addition of water (60 μL), 4 M NaOH (60 μL), and more water (180 μL). On stirring the resultant suspension at rt for 1 h, the mixture was dried (Na₂SO₄), filtered, and the filter cake washed with THF (50 mL). The combined filtrates were evaporated in vacuo to afford a 1:1 mixture of the title compounds as a pale orange oil: LC/MS t_{R} 0.29 min; MS (ES+) m/z 274; ¹H NMR δ_{H} (250 MHz, CDCl₃) 7.14-7.45 (5H, m), 5.67-5.87 (1H, m), 5.01-5.21 (2H, m), 3.51-3.71 (2H, m), 3.01 (1H, m), 2.69-2.97 (6H, m), 2.67 (0.5H, br s), 2.62 (0.5H, br s), 2.23 (3H, s), 1.90 (0.5H, dd), 1.86 (0.5H, dd), 1.75 (0.5H, dd), 1.72 (1H, br s), 1.54 (0.5H, dd), 0.99 and 0.94 (3H, 2 d).

### Example 254

### [(2R)-4-[(1R)-2-(Methyl-2-propen-1-ylamino)-1-phenylethyl]-2-methyl-1-piperazinyl][5-(4-methoxyphenyl)-6-methyl-7-(trifluoromethyl)pyrazolo[1,5-a]pyrimidin-3-yl]methanone (369) and [(2R)-4-[(1S)-2-(Methyl-2-propen-1-ylamino)-1-phenylethyl]-2-methyl-1-piperazinyl][5-(4-methoxyphenyl)-6-methyl-7-(trifluoromethyl)pyrazolo[1,5-a]pyrimidin-3-yl]methanone (370)

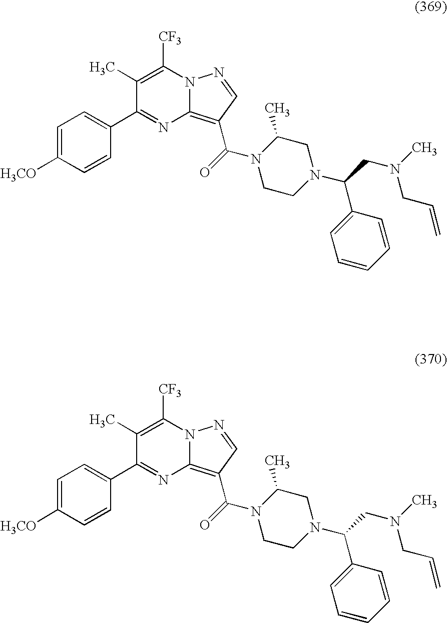

Carboxylic acid 19 (257 mg, 0.73 mmol) was treated with HATU (334 mg, 0.88 mmol), DIPEA (0.18 mL, 1.02 mmol, d 0.742) and a 1:1 mixture of piperazines 367 and 368 (210 mg, 0.77 mmol) in accordance with Method G to afford a 1:1 mixture of the title compounds after column chromatography (silica gel, 0-1% MeOH in EtOAc) as a vitreous yellow solid: LC/MS t_{R} 1.67 min; MS (ES+) m/z 304, 607; ¹H NMR δ_{H} (250 MHz, CDCl₃) 8.39 (1H, s), 7.49 (2H, d), 7.16-7.35 (5H, m), 7.02 (2H, d), 5.64-5.88 (1H, m), 5.02-5.19 (2H, m), 4.17-4.93 (1H, br s), 3.93 (3H, s), 3.90 (1H, obs m), 3.54 (1H, m), 3.43 (1H, br s), 2.95-3.06 (2H, m), 2.57-2.90 (3H, m), 2.53 (3H, q), 2.53 (1H, obs br s), 2.35-2.43 (1H, m), 2.24 and 2.21 (3H, 2 s), 2.00-2.17 (1H, m), 1.38 and 1.33 (3H, 2 d).

### Example 255

### [(2R)-4-[(1R)-2-(Methylamino)-1-phenylethyl]-2-methyl-1-piperazinyl][5-(4-methoxyphenyl)-6-methyl-7-(trifluoromethyl)pyrazolo[1,5-a]pyrimidin-3-yl]methanone (361) and [(2R)-4-[(1S)-2-(Methylamino)-1-phenylethyl]-2-methyl-1-piperazinyl][5-(4-methoxyphenyl)-6-methyl-7-(trifluoromethyl)pyrazolo[1,5-a]pyrimidin-3-yl]methanone (362)

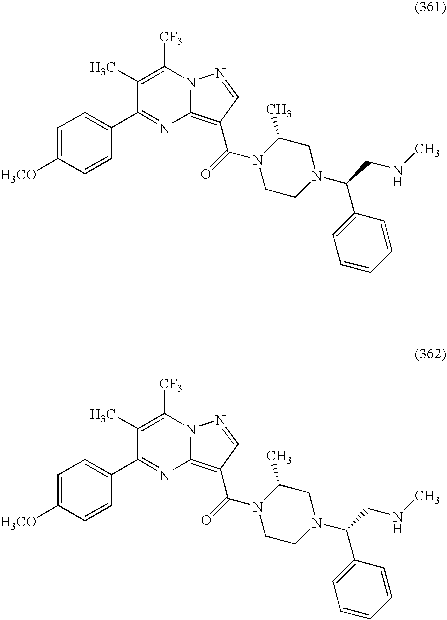

A 1:1 mixture of allyl amines 369 and 370 (100 mg, 0.165 mmol) was treated with 1,3-dimethylbarbituric acid (26 mg, 0.105 mmol) and Pd(PPh₃)₄ (19 mg, 0.017 mmol) employing the procedure of Method E to give the title compound after column chromatography (silica gel, 0-4% MeOH in DCM with 1% TEA) as a vitreous yellow solid: LC/MS t_{R} 1.61 min; MS (ES+) m/z 284, 567; ¹H NMR δ_{H} (250 MHz, CDCl₃) 8.39 and 8.38 (1H, 2 s), 7.47 (2H, d), 7.26-7.37 (3H, m), 7.14-7.21 (2H, m), 7.02 (2H, d), 4.02-4.84 (1H, br s), 3.93 (3H, s), 3.64 (1H, m), 3.04-3.44 (4H, m), 2.60-2.98 (3H, m), 2.53 (3H, q), 2.48 (3H, d), 2.35 (1H, m), 2.07 (0.5H, dd), 1.94 (0.5H, app td), 1.41 and 1.34 (3H, 2 d).

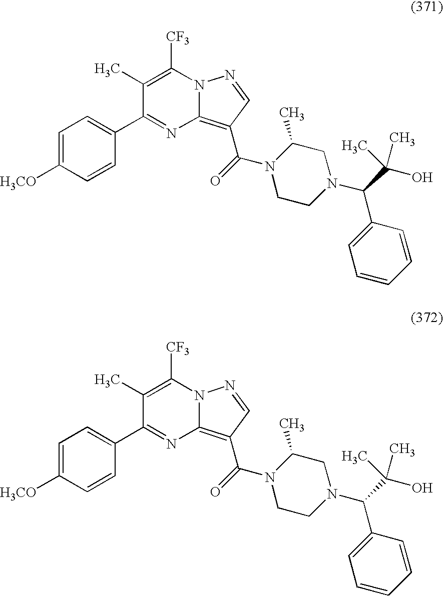

### Example 256

### (βR,3R)-α,α,3-Trimethyl-β-phenyl-1-piperazineethanol (373) and (βS,3R)-α,α,3-Trimethyl-β-phenyl-1-piperazineethanol (374)

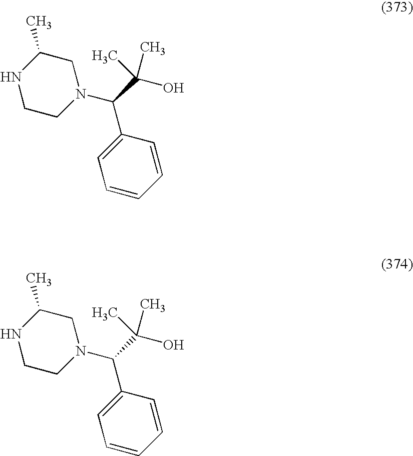

A stirred solution of a 1:1 mixture of ethyl esters 349 and 350 (150 mg, 0.57 mmol) in THF (5 mL) at 0° C. was treated with a solution of methyl magnesium bromide (1.35 mL, 1.89 mmol, 1.4 M in PhMe/THF) dropwise over 5 min. The reaction was stirred at this temperature for 45 min before reaction progress was assessed by LC/MS. A further aliquot of methyl magnesium bromide (0.82 mL, 1.14 mmol, 1.4 M in PhMe/THF) was added and stirring continued for 16 h at rt. The reaction was quenched by the addition of satd NH₄Cl (10 mL) and extracted with EtOAc (3×10 mL). The combined EtOAc phases were dried (Na₂SO₄) and filtered and the filtrate reduced in vacuo. Column chromatography (silica gel, 0-3% MeOH in DCM with 1% TEA) afforded a 1:1 mixture of the title compounds as a viscous, pale brown oil: LC/MS t_{R} 0.98 min; MS (ES+) m/z 249; ¹H NMR δ_{H} (250 MHz, CDCl₃) 7.27 (5H, app br s), 5.60 (1H, s), 3.93-4.80 (1H, br s), 3.28 and 3.27 (1H, 2 s), 2.87-3.18 (4H, m), 2.80 (1H, m), 2.57 (0.5H, app td), 2.25 (0.5H, dd), 2.11 (0.5H, app td), 1.84 (0.5H, dd), 1.19 (6H, s), 1.14 and 1.12 (3H, d).

### Example 257

### [(2R)-4-[(1R)-2-Hydroxy-2-methyl-1-phenylpropyl]-2-methyl-1-piperazinyl][5-(4-methoxyphenyl)-6-methyl-7-(trifluoromethyl)pyrazolo[1,5-a]pyrimidin-3-yl]methanone (371) and [(2R)-4-[(1S)-2-Hydroxy-2-methyl-1-phenylpropyl]-2-methyl-1-piperazinyl][5-(4-methoxyphenyl)-6-methyl-7-(trifluoromethyl)pyrazolo[1,5-a]pyrimidin-3-yl]methanone (372)

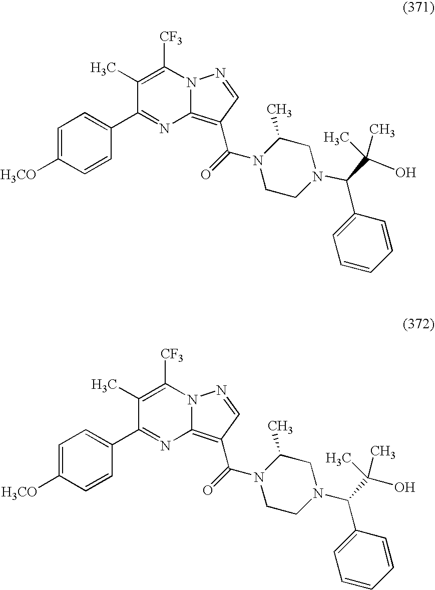

Carboxylic acid 19 (50 mg, 0.14 mmol) was treated with HATU (65 mg, 0.17 mmol), DIPEA (34 μL, 0.20 mmol, d 0.742) and a 1:1 mixture of piperazines 373 and 374 (42 mg, 0.17 mmol) in accordance with Method G to afford a 1:1 mixture of the title compounds after column chromatography (silica gel, 40-50% EtOAc in heptanes) as a vitreous yellow solid: LC/MS t_{R} 1.83 and 1.99 min; MS (ES+) m/z 582; ¹H NMR δ_{H} (250 MHz, CDCl₃) 8.37 and 8.36 (1H, 2 s), 7.45 (2H, d), 7.29 (5H, app br s), 7.01 (2H, d), 4.03-4.76 (1H, br s), 3.93 (3H, s), 3.03-3.57 (3H, m), 2.79 (1H, br s), 2.66 (1H, m), 2.53 (3H, q), 2.41-2.57 (1H, obs m), 2.14 (0.5H, dd), 1.99 (0.5H, app td), 1.46 and 1.45 (3H, 2 d), 1.18-1.34 (6H, m).

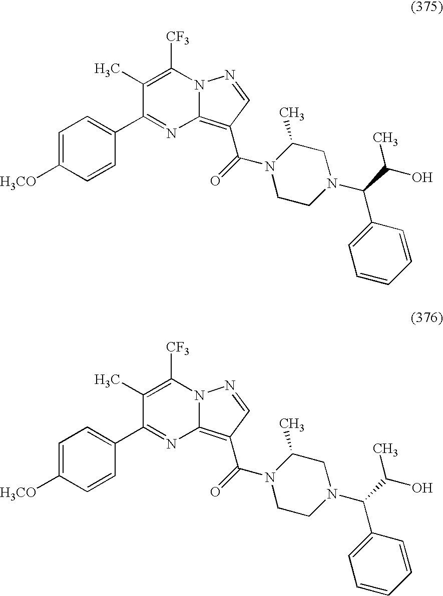

### Example 258

### (2R)-4-[(1R)-2-(Methoxymethylamino)-2-oxo-1-phenylethyl]-2-methyl-1-piperazinecarboxylic Acid, 1,1-Dimethylethyl Ester (377) and (2R)-4-[(1S)-2-(Methoxymethylamino)-2-oxo-1-phenylethyl]-2-methyl-1-piperazinecarboxylic Acid, 1,1-Dimethylethyl Ester (378)

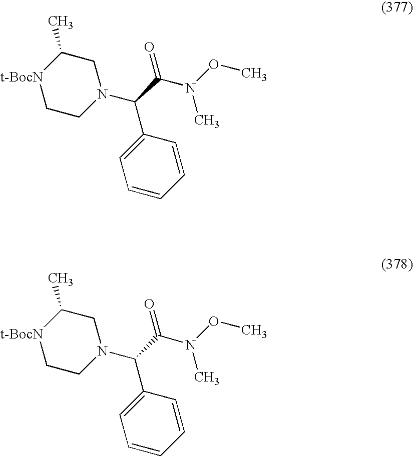

A 1:1 mixture of carboxylic acids 353 and 354 (155 mg, 0.46 mmol) was treated with HATU (209 mg, 0.55 mmol), DIPEA (0.24 mL, 1.38 mmol, d 0.742) and N,O-dimethylhydroxylamine hydrochloride (67 mg, 0.69 mmol) in accordance with Method G to afford a 1:1 mixture of the title compounds as a viscous, caramel colored oil: LC/MS t_{R} 1.47 and 1.63 min; MS (ES+) m/z 378, 400; ¹H NMR δ_{H} (250 MHz, CDCl₃) 7.43 (2H, m), 7.32 (3H, m), 4.55 and 4.49 (1H, 2 s), 4.13 (1H, m), 3.66-3.87 (1H, m), 3.52 and 3.51 (3H, 2 s), 3.16 and 3.15 (3H, 2 s), 3.00-3.24 (1H, obs m), 2.87-2.97 (1H, m), 2.68 (0.5H, m), 2.60 (0.5H, app dd), 2.45 (0.5H, dd), 2.23 (0.5H, app td), 2.08 (0.5H, dd), 2.02 (0.5H, app td), 1.43 (9H, s), 1.26 and 1.24 (3H, 2 d).

### Example 259

### (2R)-2-Methyl-4-[(1R)-2-oxo-1-phenylpropyl]-1-piperazinecarboxylic Acid, 1,1-Dimethylethyl Ester (379) and (2R)-2-Methyl-4-[(1S)-2-oxo-1-phenylpropyl]-1-piperazinecarboxylic Acid, 1,1-Dimethylethyl Ester (380)

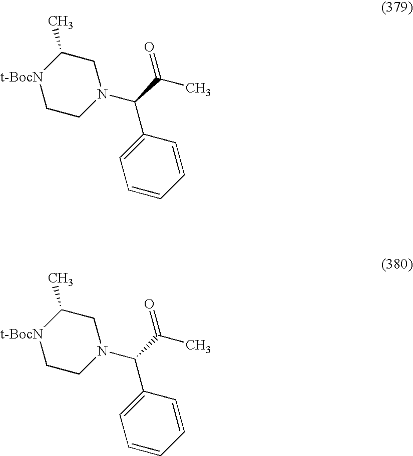

A stirred solution of a 1:1 mixture of N-methoxy-N-methyl amides 377 and 378 (186 mg, 0.49 mmol) in THF (5 mL) at −78° C. was treated with methyllithium (0.37 mL, 0.59 mmol, 1.6 M solution in Et₂O). The reaction mixture was allowed to warm to rt over 1 h before reaction progress was assessed by LC/MS. The reaction was cooled to 0° C. and a further aliquot of methyllithium (0.37 mL, 0.59 mmol, 1.6 M solution in Et₂O) added. After stirring at rt for 1 h, the reaction was quenched by the addition of satd NH₄Cl (10 mL) and extracted with EtOAc (3×10 mL). The combined EtOAc phases were dried (Na₂SO₄) and filtered and the filtrate reduced in vacuo. Column chromatography (silica gel, 10-30% EtOAc in heptanes) afforded a 1:1 mixture of the title compounds as a viscous, colorless oil: LC/MS t_{R} 1.74 and 1.85 min; MS (ES+) m/z 277, 333; ¹H NMR δ_{H} (250 MHz, CD₃OD) 7.25-7.47 (5H, m), 4.20 and 4.09 (1H, 2 m), 3.96 and 3.93 (1H, 2 s), 3.81 and 3.71 (1H, 2 br d), 3.23 (0.5H, m), 3.14 (0.5H, dd), 2.84 (0.5H, app dq), 2.72-2.63 (1H, m), 2.52 (0.5H, app dq), 2.26 (0.5H, dd), 2.12 and 2.11 (3H, 2 s), 2.04 (0.5H, app td), 1.93 (0.5H, dd), 1.77 (0.5H, app td), 1.44 (9H, s), 1.42 and 1.37 (3H, 2 d).

### Example 260

### (2R)-4-[(1R)-2-Hydroxy-1-phenylpropyl]-2-methyl-1-piperazinecarboxylic Acid, 1,1-Dimethylethyl Ester (381) and (2R)-4-[(1S)-2-Hydroxy-1-phenylpropyl]-2-methyl-1-piperazinecarboxylic Acid, 1,1-Dimethylethyl Ester (382)

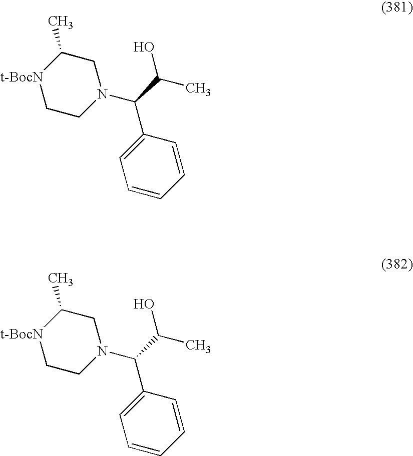

A 1:1 mixture of ketones 379 and 380 (17 mg, 0.051 mmol) was treated with sodium borohydride (2.2 mg, 0.056 mmol) in accordance with Method L. On completion, the reaction mixture was diluted with satd NH₄Cl (5 mL) and extracted into DCM (3×5 mL). The combined DCM phases were dried (Na₂SO₄) and filtered and the filtrate reduced in vacuo to afford a 1:1 mixture of the title compounds as a viscous, colorless oil: LC/MS t_{R} 1.35 min; MS (ES+) m/z 335; ¹H NMR δ_{H} (250 MHz, CDCl₃) 7.16-7.48 (5H, m), 3.94-4.50 (2H, m), 3.62-3.93 (1H, m), 2.88-3.38 (3H, m), 2.41-2.85 (2H, m), 2.28 (0.5H, dd), 2.08 (0.5H, app td), 2.00 (0.5H, dd), 1.82 (0.5H, app td), 1.38-1.49 (9H, m), 1.16-1.35 (3H, m), 0.94-1.07 (3H, m).

### Example 261

### (βR,3R)-α,3-Dimethyl-β-phenyl-1-piperazineethanol (383) and (βS,3R)-α,3-Dimethyl-α-phenyl-1-piperazineethanol (384)

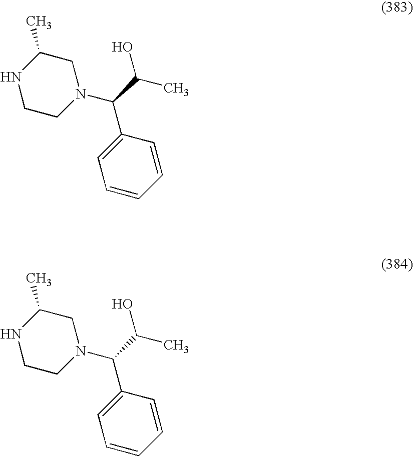

The 1:1 mixture of tert-butyl carbamates 381 and 382 (14 mg, 0.042 mmol) was treated with a solution of HCl in 1,4-dioxane (4 M) in accordance with Method X to afford a 1:1 mixture of the HCl salts of title compounds as a vitreous colorless solid: LC/MS t_{R} 0.29 min; MS (ES+) m/z 235.

### Example 262

### [(2R)-4-[(1R)-(2-Hydroxy-1-phenylpropyl)-2-methyl-1-piperazinyl][5-(4-methoxyphenyl)-6-methyl-7-(trifluoromethyl)pyrazolo[1,5-a]pyrimidin-3-yl]methanone (375) and [(2R)-4-[(1S)-(2-Hydroxy-1-phenylpropyl)-2-methyl-1-piperazinyl][5-(4-methoxyphenyl)-6-methyl-7-(trifluoromethyl)pyrazolo[1,5-a]pyrimidin-3-yl]methanone (376)

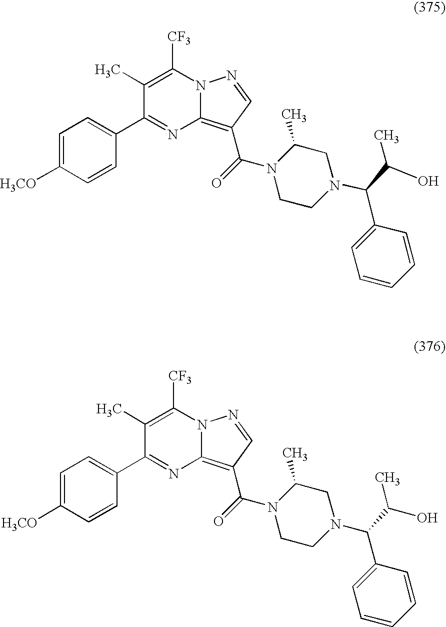

Carboxylic acid 19 (18.4 mg, 0.052 mmol) was treated with HATU (24 mg, 0.063 mmol), DIPEA (27 μL, 0.156 mmol, d 0.742) and a 1:1 mixture of piperazine HCl salts 383 and 384 (17 mg, 0.063 mmol) in accordance with Method G to afford a 1:1 mixture of the title compounds after column chromatography (silica gel, 40-70% EtOAc in heptanes, then repeated using the same conditions) as a vitreous yellow solid: LC/MS t_{R} 1.66 and 1.73 min; MS (ES+) m/z 568, 590; ¹H NMR δ_{H} (250 MHz, CDCl₃) 8.42 (1H, s), 7.41-7.52 (2H, m), 7.28-7.37 (3H, m), 7.20-7.27 (2H, m), 6.98-7.07 (2H, m), 4.35 (1H, m), 3.98-5.04 (1H, br s), 3.93 (3H, s), 3.86-3.95 (1H, obs m), 3.61-3.80 (1H, m), 3.10-3.24 (1H, m), 2.99-3.10 (1H, m), 2.36-2.63 (5.5H, m), 2.25 (0.5H, app td), 2.16 (0.5H, dd), 2.02 (1H, app td), 1.31-1.50 (3H, m), 0.95-1.06 (3H, m).

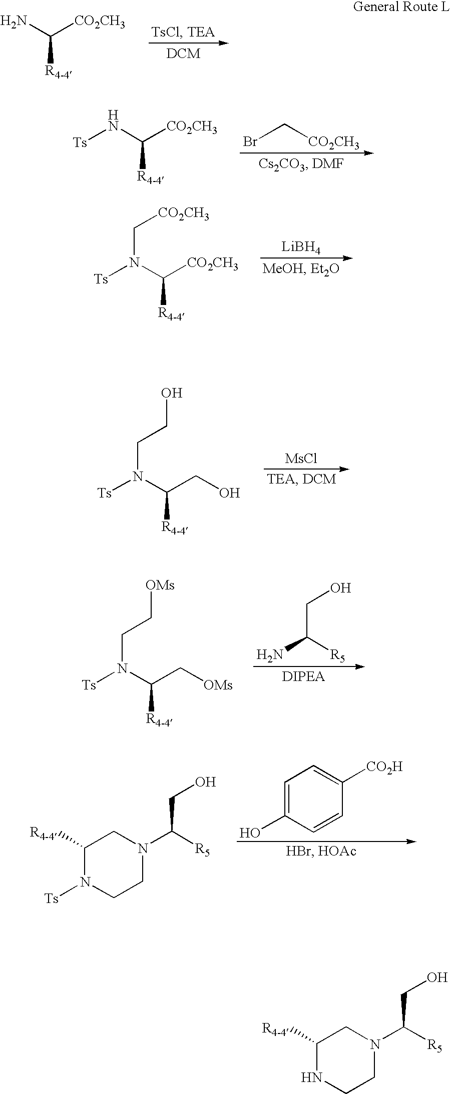

Examples of piperazines Prepared via General Route L. Alternate Route to (βR,3R)-3-Methyl-β-phenyl-1-piperazineethanol (117)

### Example 263

### N-[(4-Methylphenyl)sulfonyl]-D-alanine, Methyl Ester (385)

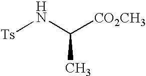

A suspension of TsCl (35.5 g, 0.188 mol) in DCM (50 mL) was added portion-wise over 30 min to a stirred suspension of D-alanine methyl ester hydrochloride (25 g, 0.179 mol) and TEA (55 mL, 0.394 mol, d 0.726) in DCM (130 mL) at 0° C. The reaction was stirred at rt for 16 h; reaction progress was monitored by LC/MS. On completion, the resultant white suspension was diluted with DCM (200 mL) and washed with water (300 mL). The aqueous phase was extracted with DCM (2×150 mL), the combined DCM phases washed with 1 M HCl (2×100 mL) and brine (50 mL), dried (MgSO₄), filtered and reduced in vacuo. Recrystallization (2:1 DCM/heptane, 100 mL) yielded the title compound as a white solid in two crops: LC/MS t_{R} 1.64 min; MS (ES+) m/z 258, 280; ¹H NMR δ_{H} (360 MHz, CDCl₃) 7.73 (2H, d), 7.30 (2H, d), 5.27 (1H, br d), 3.99 (1H, app dq), 3.54 (3H, s), 2.42 (3H, s), 1.38 (3H, d).

### Example 264

### N-(2-Methoxy-2-oxoethyl)-N-[(4-methylphenyl)sulfonyl]-D-alanine, Methyl Ester (386)

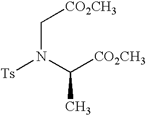

Anhydrous DMF (650 mL) was added over 30 min to a flask charged with sulfonamide 385 (41 g, 0.159 mol) and cesium carbonate (77.7 g, 0.239 mol). After stirring for an additional 10 min, methyl bromoacetate (16.6 mL, 0.175 mol, d 1.616) was added dropwise over 15 min, maintaining the internal temperature at 20-30° C. The suspension was stirred for 2 h at rt and filtered, washing the filter cake with EtOAc (200 mL). The filtrate was reduced in vacuo, the residue diluted with MTBE (300 mL) and washed with water (2×200 mL). The combined aqueous phases were extracted with MTBE (3×150 mL) and the combined MTBE phases washed with water (2×100 mL) and brine (2×100 mL). The organic phase was dried (MgSO₄), filtered and reduced in vacuo to afford the title compound as a yellow oil: LC/MS t_{R} 1.86 min; MS (ES+) m/z 330, 352; ¹H NMR δ_{H} (360 MHz, CDCl₃) 7.78 (2H, d), 7.30 (2H, d), 4.53 (1H, q), 4.30 (1H, d), 4.04 (1H d), 3.73 (3H, s), 3.54 (3H, s), 2.42 (3H, s), 1.38 (3H, d).

### Example 265

### N-(2-Hydroxyethyl)-N-[(1R)-2-hydroxy-1-methylethyl]-4-methylbenzenesulfonamide (387)

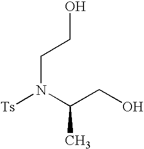

A suspension of lithium borohydride (13.9 g, 0.638 mol) in Et₂O (400 mL) at 0° C. was treated with anhydrous MeOH (25.8 mL, 0.638 mol, d 0.791) while maintaining the internal temperature at 0-5° C. After stirring for 10 min, a solution of diester 386 (52.5 g, 0.159 mol) in Et₂O (200 mL) was added via cannula over 1 h while maintaining the internal temperature below 20° C. The reaction was stirred at rt for a further 2 h before carefully quenching by the slow addition of water (300 mL) with external cooling (ice/water bath). The resultant biphasic mixture was diluted with EtOAc (200 mL) and 1 M HCl (200 mL), the organic phase separated and the aqueous layer extracted with EtOAc (3×200 mL). The combined organic phases were washed with brine (250 mL), dried (MgSO₄), filtered and reduced in vacuo to yield a white amorphous solid. Recrystallization (1:2 DCM/heptane, 400 mL) gave the title compound as small white prisms: LC/MS t_{R} 1.41 min; MS (ES+) m/z 274, 296; ¹H NMR δ_{H} (360 MHz, CDCl₃) 7.73 (2H, d), 7.30 (2H, d), 3.82-4.12 (3H, m), 3.62-3.81 (2H, m), 3.35-3.61 (3H, m), 3.12 (1H, ddd), 2.42 (3H, s) 0.81 (3H, d).

### Example 266

### 4-Methyl-N-[(1R)-1-methyl-2-[(methylsulfonyl)oxy]ethyl]-N-[2-[(methylsulfonyl)oxy]ethyl]benzenesulfonamide (388)

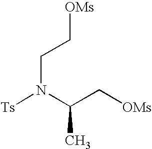

A solution of MsCl (24.2 mL, 0.312 mol, d 1.48) in DCM (50 mL) was added dropwise over 20 min to a stirred solution of diol 387 (39 g, 0.143 mol) and TEA (45.5 mL, 0.327 mol, d 0.726) in DCM (230 mL) at 0° C. Stirring was continued at rt for a further 2 h before quenching the reaction with 1 M HCl (200 mL). The aqueous phase was isolated and extracted with DCM (3×100 mL). The combined organic phases were washed with satd NaHCO₃ (200 mL) and brine (200 mL), dried (MgSO₄) and reduced in vacuo to yield the title product as a viscous, yellow oil: LC/MS t_{R} 1.81 min; MS (ES+) m/z 430, 452; ¹H NMR δ_{H} (360 MHz, CDCl₃) 7.72 (2H, d), 7.34 (2H, d), 4.43 (2H t), 4.11-4.27 (3H, m), 3.51 (1H, app dt), 3.37 (1H, app dt), 3.07 (3H, s), 3.03 (3H, s), 2.44 (3H, s), 1.04 (3H, d).

### Example 267

### (βR,3R)-3-Methyl-4-[(4-methylphenyl)sulfonyl]-α-phenyl-1-piperazineethanol (389)

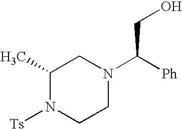

A suspension of (bis)mesylate 388 (61 g, 0.142 mol) and (R)-(−)-2-phenylglycinol (20.5 g, 0.149 mol) in DIPEA (62 mL, 0.355 mol, d 0.742) was heated at 140° C. for 4 h. Reaction progress was monitored by LC/MS. After cooling to rt, the reaction mixture was diluted with DCM (300 mL) and washed with satd NaHCO₃ (400 mL). The aqueous phase was isolated, extracted with DCM (3×100 mL) and the combined organic phases washed with brine (250 mL) before drying (MgSO₄) and concentrating in vacuo. The residue thus obtained was dissolved in DCM (200 mL) and MTBE (300 mL) and washed with 2 M HCl (1×300 mL, 5×100 mL). The combined aqueous phases were neutralized with solid NaHCO₃ to pH 8-9 and extracted with EtOAc (4×300 mL). The combined EtOAc phases were washed with brine (300 mL), dried (MgSO₄), filtered and reduced in vacuo to yield the title compound as a viscous red oil, which slowly solidified on standing at rt to a red wax: LC/MS t_{R} 1.40 min; MS (ES+) m/z 375, 397; ¹H NMR δ_{H} (360 MHz, CDCl₃) 7.63 (2H, d), 7.28-7.35 (3H, m), 7.23 (2H, d), 7.07-7.14 (2H, m), 4.05-4.10 (1H, m), 3.91 (1H, dd), 3.56-3.70 (3H, m), 3.29 (1H, app td), 2.78 (1H, app dq), 2.47-2.73 (1H, br s), 2.52 (1H, app dt), 2.40 (3H, s), 2.30 (1H, app td), 1.95 (1H, dd), 1.15 (3H, d).

### Example 268

### (βR,3R)-3-Methyl-β-phenyl-1-piperazineethanol (117)

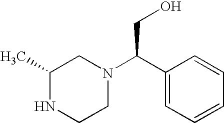

A suspension of 4-hydroxybenzoic acid (48.6 g, 0.352 mol) and N-tosyl piperazine 389 (47 g, 0.125 mol) in 33% HBr in AcOH (150 mL) was heated at 70° C. for 3 h; reaction progress was monitored by LC/MS. After allowing to cool to rt, the reaction mixture was diluted with water (150 mL) and resultant precipitate isolated by filtration, washing the filter cake with water (500 mL). The O-acetate thus prepared was treated with 50% NaOH (250 mL) at 0° C. and stirred at rt for 1.5 h before extracting the reaction mixture with EtOAc (1×300 mL, 3×150 mL) followed by 3:1 CHCl₃/i-PrOH (3×200 mL). The combined organic phases were washed with brine (150 mL), dried (MgSO₄), filtered and reduced in vacuo to yield the title compound as a viscous caramel colored oil with spectral data consistent to that of 117 as prepared in Example 119.

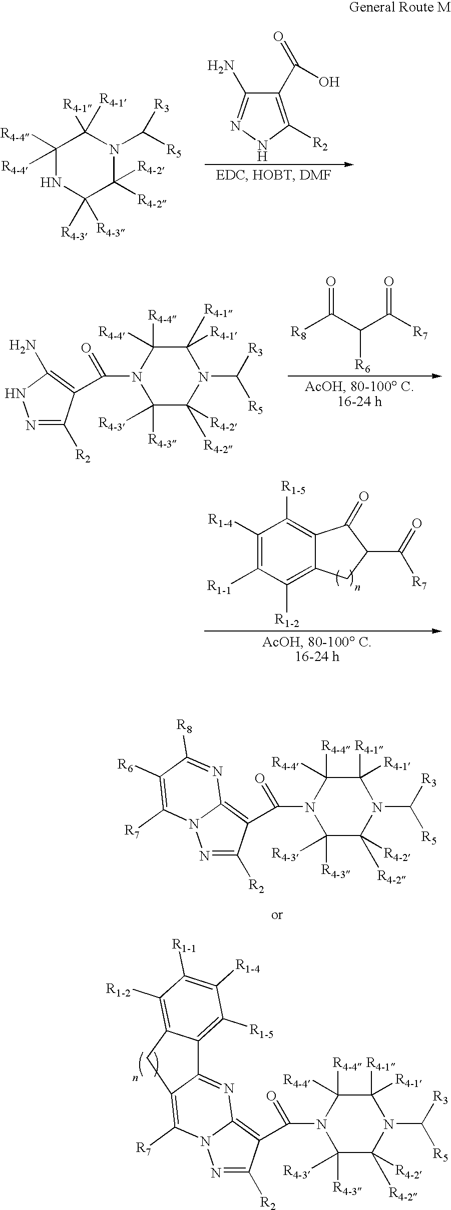

### Description of Methods Used in General Route M

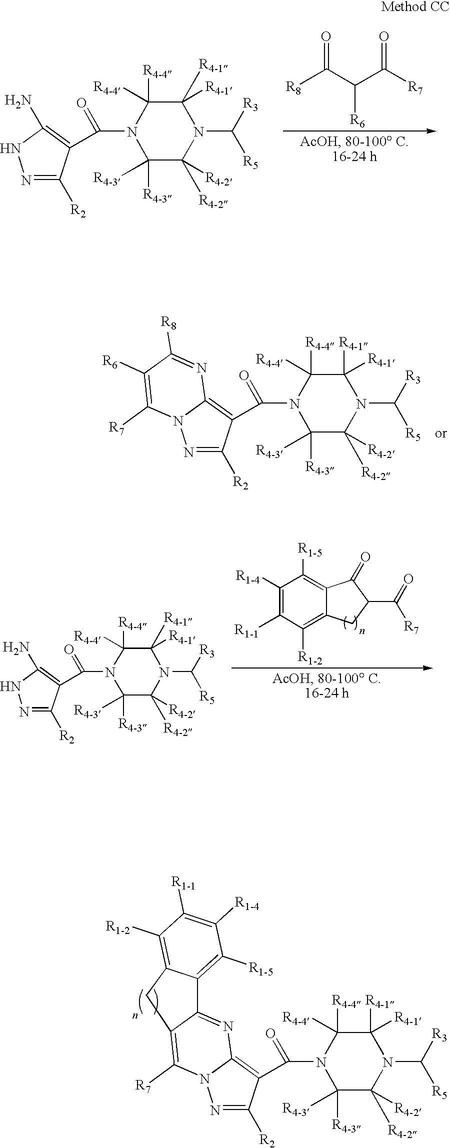

For R₃=CH₂OH:

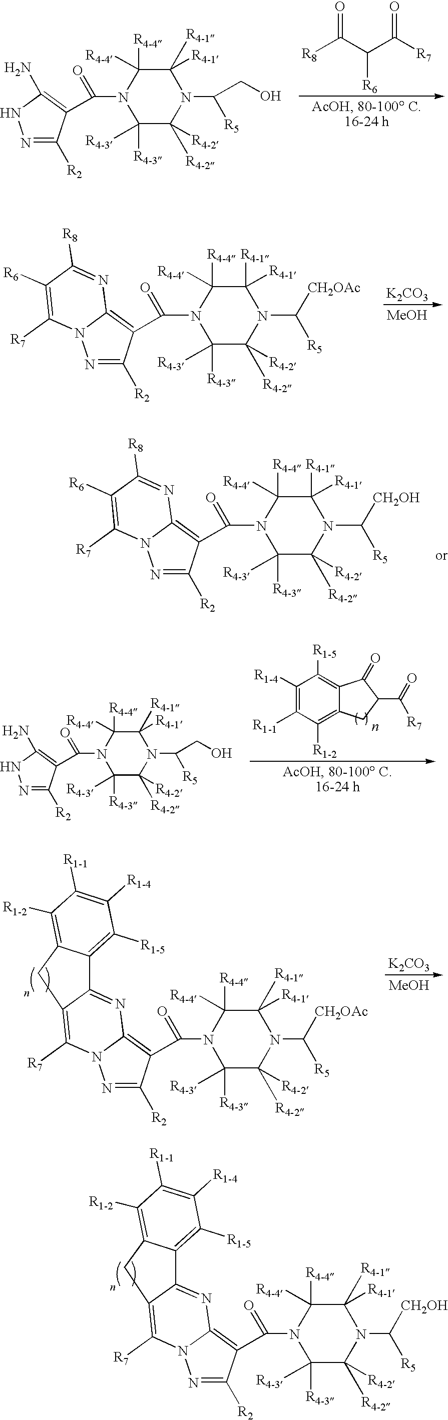

The 3-aminopyrazole amide (1 equiv) and diketone (1.1 equiv) in AcOH (50 vol) was heated at 80-100° C. for 16-24 h. Reaction progress was monitored by LC/MS. On completion the solvent was removed in vacuo and the residue purified by column chromatography (silica gel, 0-2% MeOH in DCM or 60% EtOAc in heptanes) providing the desired product. In those examples where an acetate ester is formed during the reaction, the residue was dissolved in MeOH (50 vol) and treated with K₂CO₃ (5 equiv). The resultant suspension was stirred for 2-5 h with monitoring by LC/MS. On completion the reaction mixture was filtered and the filter cake washed with EtOAc (100 vol). Reduction of the filtrate in vacuo followed by column chromatography (silica gel, 0-2% MeOH in DCM or 60% EtOAc in heptanes) gave the desired product.

### Examples of Compounds Prepared Via General Route M

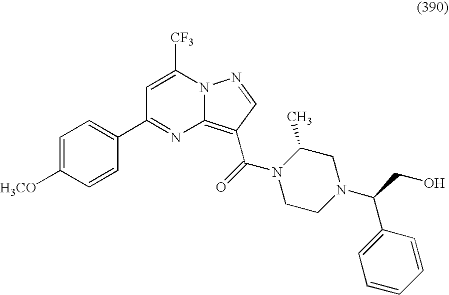

### Example 269

### (3-Amino-1H-pyrazol-4-yl)[(2R)-4-[(1R)-2-hydroxy-1-phenylethyl]-2-methyl-1-piperazinyl]methanone (391)

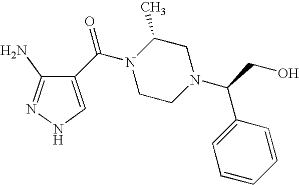

3-Amino-1H-pyrazole-4-carboxylic acid (1.38 g, 10.9 mmol) was treated with EDC (2.09 g, 10.9 mmol), HOBT (1.47 g, 10.9 mmol) and piperazine 117 (2.0 g, 9.08 mmol) in accordance with Method G (alternative procedure). Purification by column chromatography (silica gel, 0-10% MeOH in DCM) afforded the title compound as a vitreous tan solid: LC/MS t_{R} 0.29 min; MS (ES+) m/z 330, 352; ¹H NMR δ_{H} (250 MHz, CDCl₃) 7.39 (1H, s), 7.29-7.38 (3H, m), 7.12-7.24 (2H, m), 5.01 (2H, br s), 4.58 (1H, br s), 4.26 (1H, br d), 3.99 (1H, dd), 3.61-3.79 (2H, m), 3.40 (1H, br t), 2.95 (1H, d), 2.67 (1H, d), 2.52-3.08 (1H, br s), 2.36 (1H, app td), 2.00 (1H, dd), 1.40 (3H, d).

### Example 270

### [(2R)-4-[(1R)-2-(Hydroxy)-1-phenylethyl]-2-methyl-1-piperazinyl][5-(4-methoxyphenyl)-7-(trifluoromethyl)pyrazolo[1,5-a]pyrimidin-3-yl]methanone (390)

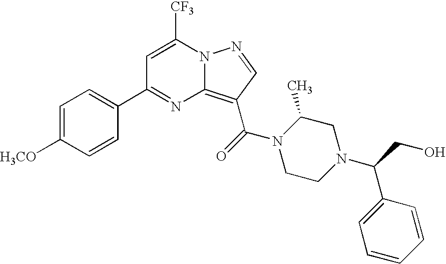

Aminopyrazole 391 (27 mg, 0.082 mmol) was treated with the potassium salt of diketone 1 (25 mg, 0.088 mmol) in accordance with Method CC to give the title compound after column chromatography (silica gel, 0-1% MeOH in DCM) as a vitreous yellow solid: LC/MS t_{R} 1.57 min; MS (ES+) m/z 540; ¹H NMR δ_{H} (250 MHz, CDCl₃) 8.43 (1H, s), 8.05 (2H, d), 7.64 (1H, s), 7.34-7.43 (3H, m), 7.17-7.24 (2H, m), 7.03 (2H, d), 4.09-4.97 (1H, br s), 3.95 (3H, s), 3.81-4.07 (2H, obs m), 3.64-3.78 (2H, m), 3.46-3.64 (1H, br s), 2.63-3.07 (3H, m), 2.54 (1H, app td), 2.20 (1H, dd), 1.44 (3H, d).

### Example 271

### [5,6-Dihydro-3-methoxy-7-(trifluoromethyl)benzo[h]pyrazolo[5,1-b]quinazolin-11-yl][(2R)-4-[(1R)-2-hydroxy-1-phenylethyl]-2-methyl-1-piperazinyl]methanone (392)

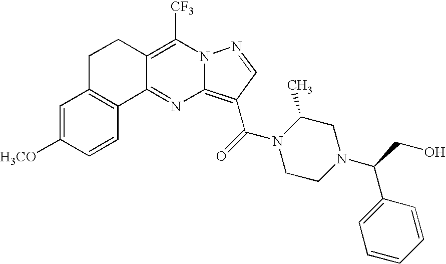

Aminopyrazole 391 (37 mg, 0.112 mmol) was treated with diketone 5 (36.5 mg, 0.134 mmol) in accordance with Method CC to give the title compound after column chromatography (silica gel, 0-1% MeOH in DCM) as a vitreous yellow solid: LC/MS t_{R} 1.65 min; MS (ES+) m/z 566; ¹H NMR δ_{H} (250 MHz, CDCl₃) 8.35 (1H, s), 8.16 (1H, d), 7.31-7.45 (3H, m), 7.13-7.25 (2H, m), 6.89 (1H, dd), 6.79 (1H, d), 4.33-5.04 (1H, br s), 3.93 (3H, s), 3.81-4.31 (2H, m), 3.65-3.81 (2H, m), 3.53 (1H, br s), 3.15-3.38 (2H, m), 2.65-3.07 (5H, m), 2.54 (1H, app td), 2.21 (1H, dd), 1.43 (3H, d).

### Example 272

### [6-Fluoro-5-(4-methoxyphenyl)-7-(trifluoromethyl)pyrazolo[1,5-a]pyrimidin-3-yl][(2R)-4-[(1R)-2-hydroxy-1-phenylethyl]-2-methyl-1-piperazinyl]methanone (393) and [(2R)-4-[(1R)-2-hydroxy-1-phenylethyl]-2-methyl-1-piperazinyl][6-methoxy-5-(4-methoxyphenyl)-7-(trifluoromethyl)pyrazolo[1,5-a]pyrimidin-3-yl]methanone (394)

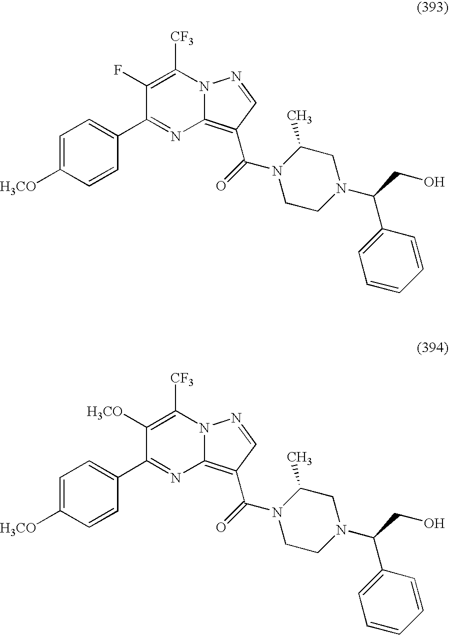

Aminopyrazole 391 (37 mg, 0.112 mmol) was treated with diketone 4 (37.7 mg, 0.134 mmol) in accordance with Method CC to afford the title compounds after column chromatography (silica gel, 0-1% MeOH in DCM) and reverse phase preparative HPLC (5-95% MeCN in water) as vitreous yellow solids: for 393, LC/MS t_{R} 1.66 min; MS (ES+) m/z 558; ¹H NMR δ_{H} (250 MHz, CDCl₃) 8.41 (1H, s), 7.99 (2H, dd), 7.31-7.44 (3H, m), 7.13-7.23 (2H, m), 7.03 (2H, d), 3.82-5.01 (2H, br m), 3.95 (3H, s), 3.13-3.79 (4H, m), 2.58-3.02 (3H, m), 2.49 (1H, app td), 2.15 (1H, dd), 1.42 (3H, d). For 394, LC/MS t_{R} 1.63 min; MS (ES+) m/z 570; ¹H NMR δ_{H} (250 MHz, CDCl₃) 8.37 (1H, s), 8.01 (2H, d), 7.30-7.40 (3H, m), 7.12-7.23 (2H, m), 7.01 (2H, d), 3.94 (3H, s), 3.88-4.84 (3H, br m), 3.64-3.72 (2H, m), 3.61 (3H, s), 3.34-3.63 (1H, br s), 2.58-3.00 (3H, m), 2.48 (1H, app td), 2.13 (1H, dd), 1.41 (3H, d).

### Example 273

### [5-(4-Ethylphenyl)-6-methyl-7-(trifluoromethyl)pyrazolo[1,5-a]pyrimidin-3-yl][(2R)-4-[(1R)-2-hydroxy-1-phenylethyl]-2-methyl-1-piperazinyl]-methanone (395)

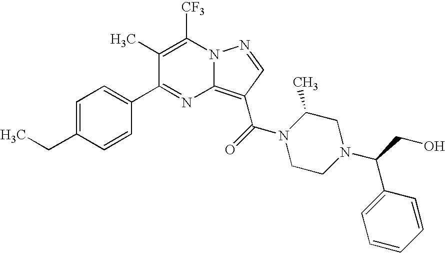

Aminopyrazole 391 (37 mg, 0.112 mmol) was treated with diketone 9 (35 mg, 0.134 mmol) in accordance with Method CC to give the title compound after column chromatography (silica gel, 0-2% MeOH in DCM; repeated using 60% EtOAc in heptanes) as a vitreous yellow solid: LC/MS t_{R} 1.71 min; MS (ES+) m/z 552; ¹H NMR δ_{H} (250 MHz, CDCl₃) 8.41 (1H, s), 7.23-7.45 (7H, m), 7.10-7.18 (2H, m), 4.14-4.93 (1H, br s), 3.89-4.02 (1H, m), 3.60-3.75 (2H, m), 3.45 (1H, br s), 2.80 (2H, q), 2.52 (3H, q), 2.40-3.00 (4H, obs m), 2.10 (1H, dd), 1.39 (3H, d), 1.35 (3H, t).

### Example 274

### [5-(4-Cyclopropylphenyl)-6-methyl-7-(trifluoromethyl)pyrazolo[1,5-a]pyrimidin-3-yl][(2R)-4-[(1R)-2-hydroxy-1-phenylethyl]-2-methyl-1-piperazinyl]-methanone (396)

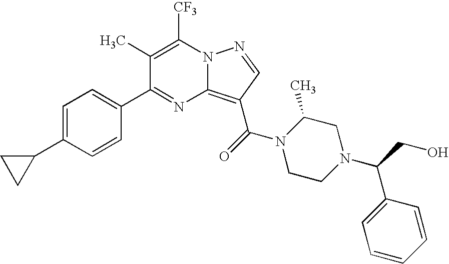

Aminopyrazole 391 (45.6 mg, 0.138 mmol) was treated with diketone 7 (44.8 mg, 0.166 mmol) in accordance with Method CC to give the title compound after column chromatography (silica gel, 60% EtOAc in heptanes) as a vitreous yellow solid: LC/MS t_{R} 1.72 min; MS (ES+) m/z 564; ¹H NMR δ_{H} (250 MHz, CDCl₃) 8.41 (1H, s), 7.27-7.50 (5H, m), 7.02-7.25 (4H, m), 4.08-5.01 (1H, br m), 3.88-4.04 (1H, m), 3.60-3.80 (2H, m), 3.46 (1H, br s), 2.86 (2H, br s), 2.65 (1H, br d), 2.52 (3H, q), 2.31-2.55 (1H, obs m), 2.09 (1H, dd), 1.95-2.05 (1H, m), 1.38 (3H, d), 1.01-1.19 (2H, m), 0.77-0.91 (2H, m).

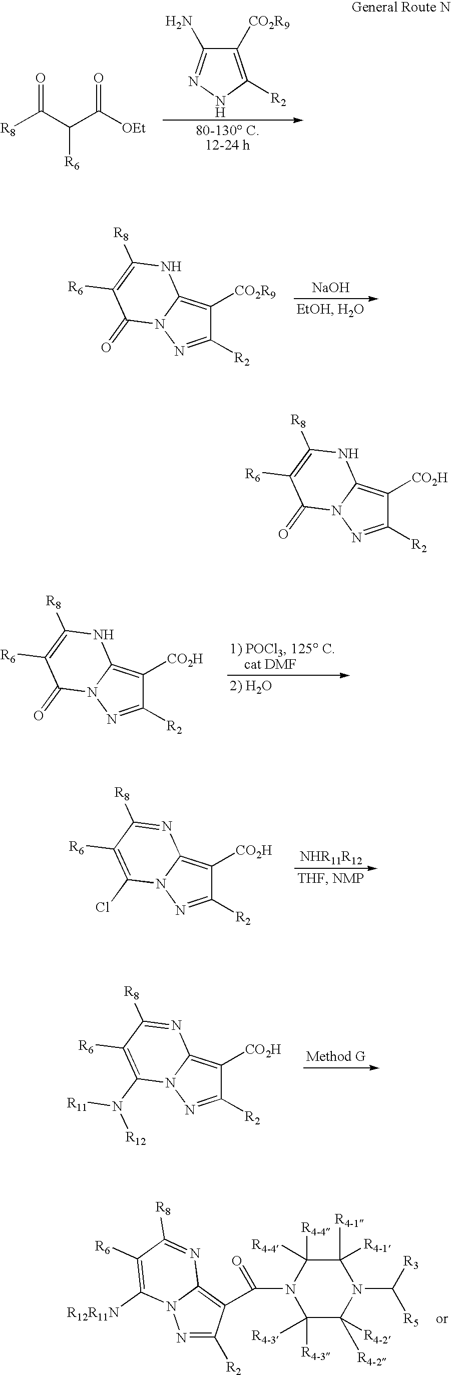

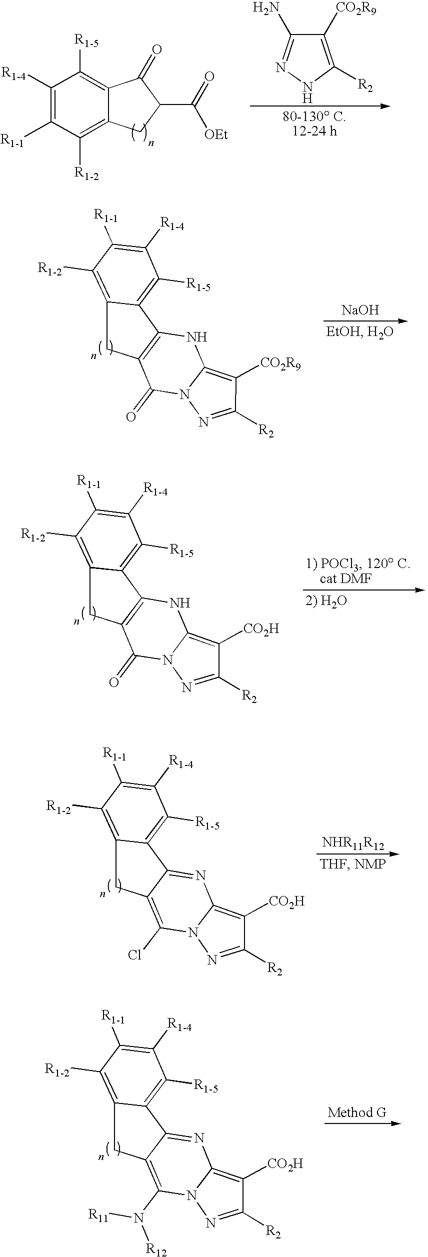

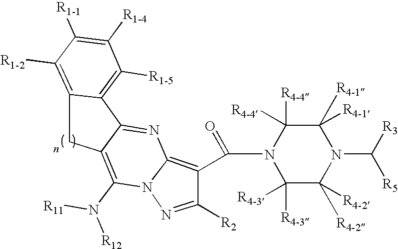

### Description of Methods Used in General Route N

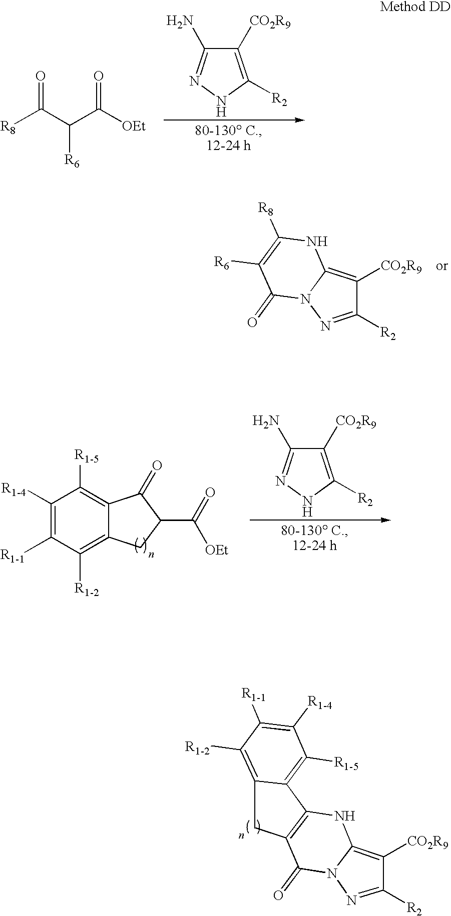

A stirred solution of the β-ketoester (1-1.1 equiv) and 3-amino-1H-pyrazole-4-carboxylic ester (1 equiv) in AcOH (10 vol) was heated at 80 to 130° C. for 12-24 h. On cooling to rt, the reaction mixture was diluted with MTBE (50 vol). The resultant precipitate (acetylated aminopyrazole) was removed by filtration, washed with MTBE (10 vol) and the filtrate evaporated. The residue obtained from the filtrate was recrystallized (MeOH) to give the desired product.

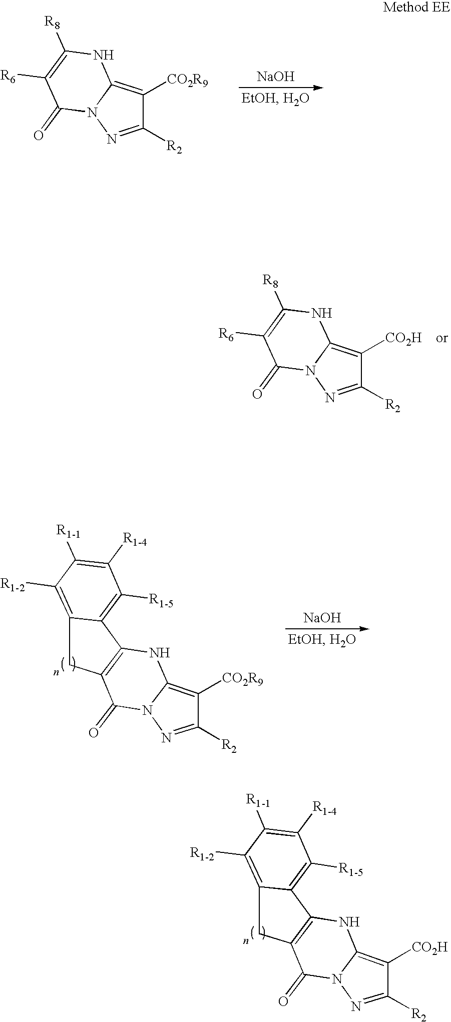

A stirred solution of the pyrazolopyrimidinone ester in EtOH (40 vol) was treated with 4 M NaOH (40 vol) and heated to 70° C. for 4-16 h, or until the reaction had reached completion by LC/MS. On cooling to rt, the EtOH was evaporated to a small volume and the product that precipitated was filtered. If recovery was poor, the filtrate was taken to pH 4 by the addition of 6 M HCl and the resultant precipitate again isolated by filtration to give the desired product.

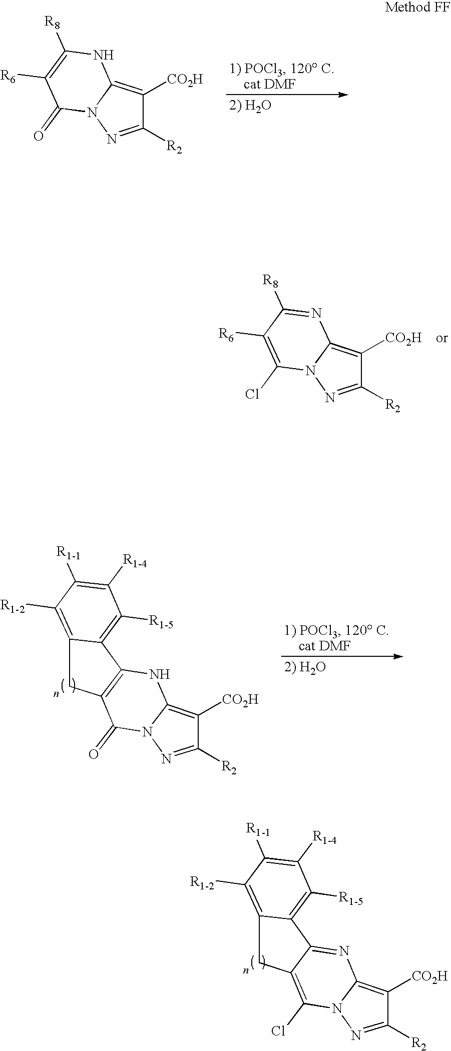

A stirred suspension of the pyrazolopyrimidinone carboxylic acid in POCl₃ (40 vol) was treated with DMF (0.3 equiv) and heated to 120° C. in a sealed tube for 3-5 days. The reaction mixture was cooled to rt and poured onto iced water (300 vol) with rapid agitation. The resultant precipitate was isolated by filtration to give the desired product.

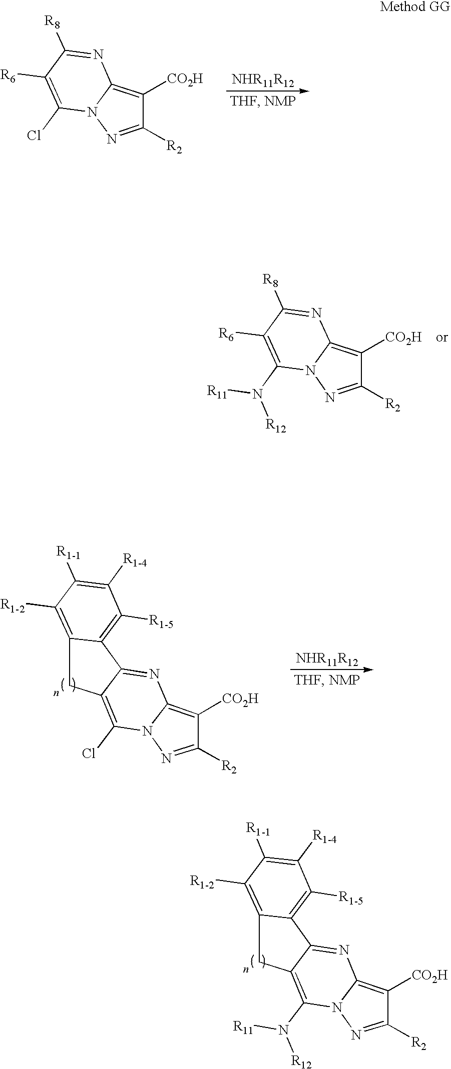

To a stirred rt solution of the pyrazolopyrimidine carbonyl chloride (1 equiv) in THF (40 vol) and NMP (20 vol) was added a solution of the desired amine (5 equiv) in THF (8 vol). Reaction progress was monitored by LC/MS. On completion, the THF was removed in vacuo and the residue diluted with water (40 vol) and treated with 6 M HCl (20 vol). The resultant precipitate was isolated by filtration, washed with water (40 vol), and dried to give the desired product.

### Examples of Compounds Prepared Via General Route N

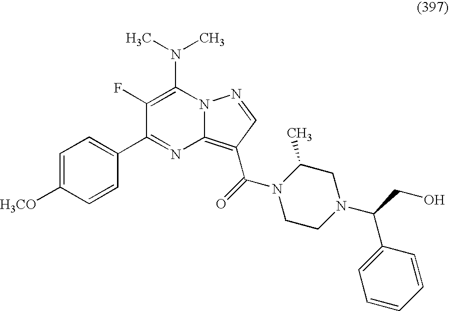

### Example 275

### 2-Fluoro-3-(4-methoxyphenyl)-3-oxopropanoic Acid, Ethyl Ester (398)

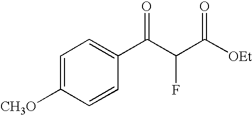

Selectfluor™ (17.8 g, 50.4 mmol) was added to a stirred rt solution of ethyl (4-methoxybenzoyl)acetate (10 mL, 45 mmol) and TiCl₄ (0.25 mL, 2.25 mmol) in MeCN (520 mL). After 4.5 h at rt, the reaction mixture was diluted with MTBE (2 L) and filtered through celite before reducing the filtrate in vacuo. The residue thus obtained was re-suspended in MTBE (50 mL) and again filtered through celite. The filtrate was evaporated to dryness to give the title compound as a 32:1 mixture with the bis-fluorinated product: LC/MS t_{R} 1.94 min; MS (ES+) m/z 241.

### Example 276

### 4,7-Dihydro-6-fluoro-5-(4-methoxyphenyl)-7-oxopyrazolo[1,5-a]pyrimidine-3-carboxylic Acid, Methyl Ester (399)

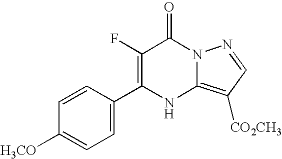

Methyl 3-amino-1H-pyrazole-4-carboxylate 14 (4.93 g, 35 mmol) was treated with β-ketoester 398 (7 g, 29.1 mmol) using Method DD to give the title compound: LC/MS t_{R} 1.66 min; MS (ES+) m/z 318, 635.

### Example 277

### 4,7-Dihydro-6-fluoro-5-(4-methoxyphenyl)-7-oxopyrazolo[1,5-a]pyrimidine-3-carboxylic Acid (400)

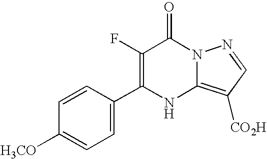

Ester 399 (2.7 g, 6.86 mmol) was hydrolyzed using Method EE to give the title compound as the HCl salt: LC/MS t_{R} 1.48 min; MS (ES+) m/z 304, 607.

### Example 278

### 7-Chloro-6-fluoro-5-(4-methoxyphenyl)pyrazolo[1,5-a]pyrimidine-3-carboxylic Acid (401)

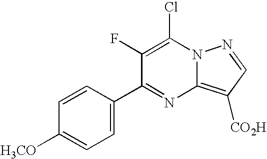

Carboxylic acid 400 (1.07 g, 1.11 mmol) was treated with POCl₃ (12.5 mL, 0.13 mol) using Method FF to give the title compound: LC/MS t_{R} 1.89 min; MS (ES+) m/z 322, 324.

### Example 279

### 7-Dimethylamino-6-fluoro-5-(4-methoxyphenyl)pyrazolo[1,5-a]pyrimidine-3-carboxylic Acid (402)

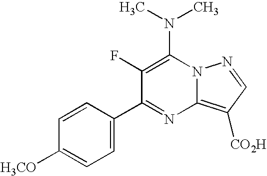

Chloropyrazolopyrimidine carboxylic acid 401 (25 mg, 0.078 mmol) was treated with dimethylamine (0.19 mL, 0.38 mmol, 2 M solution in THF) using Method GG to give the title compound as a pale orange powder: LC/MS t_{R} 1.87 min; MS (ES+) m/z 313, 331; ¹H NMR δ_{H} (250 MHz, DMSO-d₆) 12.10 (1H, br s), 8.48 (1H, s), 7.96 (2H, d), 7.13 (2H, d), 3.85 (3H, s), 3.32 (6H, obs s).

### Example 280

### [7-(Dimethylamino)-6-fluoro-5-(4-methoxyphenyl)pyrazolo[1,5-a]pyrimidin-3-yl][(2R)-4-[(1R)-2-hydroxy-1-phenylethyl]-2-methyl-1-piperazinyl]methanone (397)

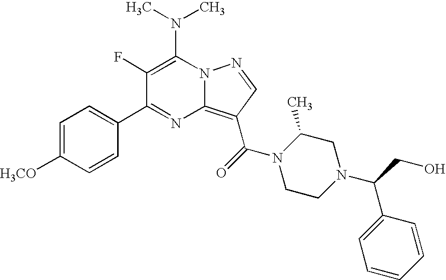

Carboxylic acid 402 (6.5 mg, 0.020 mmol) was treated with HATU (9.0 mg, 0.024 mmol), DIPEA (11 μL, 0.059 mmol, d 0.742) and piperazine 117 (4.8 mg, 0.022 mmol) in accordance with Method G to afford the title compound after column chromatography (silica gel, 0-2% MeOH in DCM) as a vitreous yellow solid: LC/MS t_{R} 1.55 min; MS (ES+) m/z 267, 533; ¹H NMR δ_{H} (250 MHz, CDCl₃) 8.32 (1H, s), 7.92 (2H, d), 7.33-7.38 (3H, m), 7.17-7.22 (2H, m), 6.99 (2H, d), 3.93 (3H, s), 3.83-4.82 (3H, br m), 3.68-3.74 (2H, m), 3.48 (1H, br s), 3.37 (3H, s), 3.36 (3H, s), 2.91 (2H, br s), 2.70 (1H, br d), 2.56 (1H, app td), 2.21 (1H, br d), 1.40 (3H, d).

### Example 281

### LC Method

LC analysis was performed using either a Waters Atlantis dC18 2.1×50 mm (5 μM resin) column eluting with 0.1% formic acid in 5-100% acetonitrile in water at a flow rate of 1 mL/min or a Waters Atlantis dC18 2.1×100 mm (3 μM resin) column at 40° C. eluting with 0.1% formic acid in 5-100% acetonitrile in water at a flow rate of 0.6 mL/min. The 1 mL/min column had a run time of 3.5 minutes and was used when the fractions were easily separable. The 0.6 mL/min had a run time of 7 min, and was used when better resolution was required.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. All publications, patent applications and patents cited herein are fully incorporated by reference.

1. A compound according Formula I: 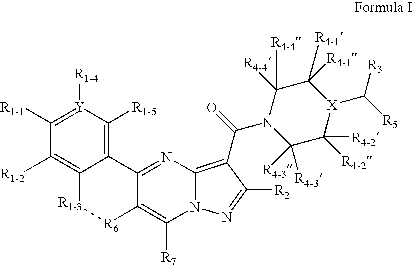
or a pharmaceutically acceptable salt or solvate thereof; wherein:
R₁₋₁ is selected from the group consisting of hydrido, hydroxyl, alkoxy, alkyl, cycloalkyl, dialkylamino, halo, haloalkyl, haloalkoxy, cyanoalkoxy, and nitro;
R₁₋₂ is selected from the group consisting of hydrido, hydroxyl, alkoxy, alkyl, and halo; or
R₁₋₁ and R₁₋₂ may be taken together in conjunction with the ring to which they are attached to form a heterocycle selected from the group consisting of 1,3-dioxolanyl, 1,4-dioxanyl, pyranyl, and 2,3-dihydrofuranyl;
R₁₋₃ is selected from the group consisting of hydrido, hydroxyl, alkoxy, alkyl, and halo when the dashed bond between R₁₋₃ and R₆ is not present or R₁₋₃ and R₆ are taken together to form (CHR₁₈)ₘ where m is 0, 1, or 2 when the dashed bond between R₁₋₃ and R₆ is present;
R₁₋₄ is selected from the group consisting of hydrido, hydroxyl, alkoxy, alkyl, and halo, with the proviso that R₁₋₄ is not present when Y is N;
R₁₋₅ is selected from the group consisting of hydrido, hydroxyl, alkoxy, alkyl, and halo;
R₂ is selected from the group consisting of hydrido, halo, hydroxyl, cyano, alkyl, alkoxy, alkoxyalkyl, alkoxyalkanoyl, alkoxycarbonyl, amido, alkylamino, alkylamido, alkylaminoalkyl, alkylamidoalkyl, cycloalkyl, hydroxyalkyl, aminoalkyl, alkylaminoalkyl, carboxyl, alkoxycarbonyl, aminocarbonyl, and arylalkyl;
R₃ is selected from the group consisting of alkyl, cyano, haloalkyl, hydroxyalkyl, alkoxyalkyl, aminoalkyl, alkylaminoalkyl, dialkylaminoalkyl, carboxyl, alkoxycarbonyl, aminocarbonyl, alkylaminocarbonyl, dialkylaminocarbonyl, alkylcarbonylaminoalkyl, and alkylsulfonylaminoalkyl;
R₄₋₂′ and R₄₋₂″ are independently selected from the group consisting of hydrido, alkyl, cycloalkyl, hydroxyalkyl, alkoxyalkyl, aminoalkyl, alkylaminoalkyl, dialkylaminoalkyl, carboxyl, alkoxycarbonyl, aminocarbonyl, alkylaminocarbonyl, dialkylaminocarbonyl, arylalkyl, arylalkoxyalkyl, alkylaryl, haloarylalkyl, and haloalkylaryl; or R₄₋₂′ and R₄₋₂″ may be taken together to form a 3-8 membered carbocycle or a heterocycle;
R₄₋₄′ and R₄₋₄″ are independently selected from the group consisting of hydrido, alkyl, cycloalkyl, hydroxyalkyl, alkoxyalkyl, aminoalkyl, alkylaminoalkyl, dialkylaminoalkyl, carboxyl, alkoxycarbonyl, aminocarbonyl, alkylaminocarbonyl, dialkylaminocarbonyl, arylalkyl, alkylaryl, haloarylalkyl, and haloalkylaryl; or R₄₋₄′ and R₄₋₄″ may be taken together to form a 3-8 membered carbocycle or a heterocycle;
R₄₋₁′, R₄₋₁″, R₄₋₃′ and R₄₋₃″ are independently selected from the group consisting of hydrido, and alkyl, or R₄₋₁′, R₄₋₁″ may be taken together to form an oxo, or R₄₋₃′ and R₄₋₃″ may be taken together to form an oxo; or
any two of R₄₋₁′, R₄₋₁″, R₄₋₂′, R₄₋₂″, R₄₋₃′, R₄₋₃″, R₄₋₄′ and R₄₋₄″ may be taken together to form a 3-8 membered carbocycle or heterocycle;
R₅ is an optionally substituted C₆-C₁₀ aryl, or optionally substituted heteroaryl, wherein said heteroaryl comprises 1, 2, 3, or 4 heteroatoms independently selected from N, O and S;
R₆ is selected from the group consisting of hydrido, halo, alkyl, alkoxy, alkoxyalkyl, alkoxyalkanoyl, alkoxycarbonyl, amido, alkylamino, alkylamido, alkylaminoalkyl, alkylamidoalkyl, cycloalkyl, cyano, hydroxyl, hydroxyalkyl, aminoalkyl, alkylaminoalkyl, dialkylamino, dialkylaminoalkyl, carboxyl, alkoxycarbonyl, aminocarbonyl, arylalkyl, alkylaryl, haloarylalkyl, haloalkylaryl, alkylthio, alkylsulfonyl, and alkylsulfinyl when the dashed bond between R₁₋₃ and R₆ is not present, or R₁₋₃ and R₆ are taken together to form (CHR₁₈)ₘ where m is 0, 1, or 2 when the dashed bond between R₁₋₃ and R₆ is present;
R₇ is selected from the group consisting of hydrido, alkyl, alkoxy, alkoxyalkyl, alkoxyalkanoyl, alkoxycarbonyl, amido, alkylamino, alkylamido, alkylaminoalkyl, alkylamidoalkyl, cycloalkyl, hydroxyl, hydroxyalkyl, haloalkyl, aminoalkyl, alkylaminoalkyl, alkyl ester, carboxamido, dialkylamino, dialkylaminoalkyl, carboxyl, alkoxycarbonyl, aminocarbonyl, arylalkyl, alkylaryl, haloarylalkyl, haloalkylaryl, alkylthio, alkylsulfonyl, and alkylsulfinyl; and
R₁₈, in each instance, is independently selected from the group consisting of hydrido, alkyl, alkoxy, hydroxyl, and halo.

2. (canceled)

3. (canceled)

4. The compound according to claim 1 having Formula I-b: 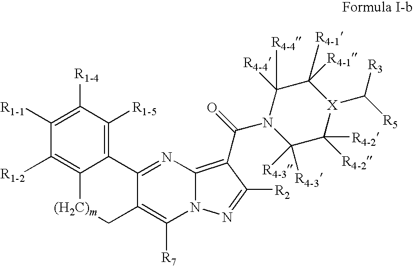
or a pharmaceutically acceptable salt or solvate thereof.

5. The compound according to claim 1 having Formula I-c: 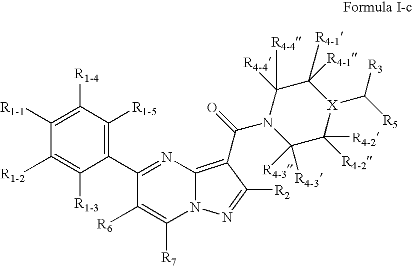
or a pharmaceutically acceptable salt or solvate thereof.

6. The compound according to claim 1 having Formula I-d-1 or 1-d-2: 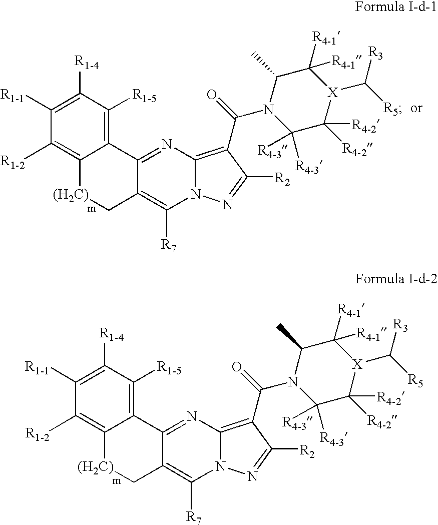
or a pharmaceutically acceptable salt or solvate thereof.

7. The compound according to claim 1 having Formula I-e-1 or Formula 1-e-2: 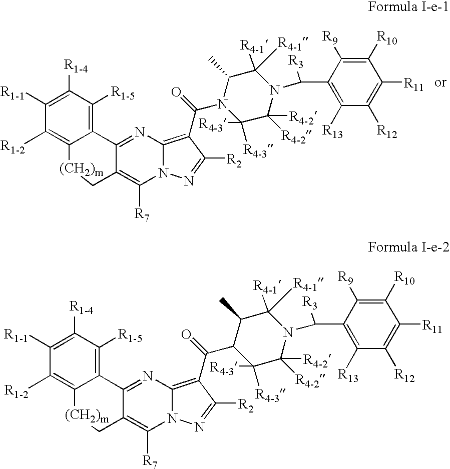
or a pharmaceutically acceptable salt or solvate thereof.

8. The compound according to claim 1 having Formula I-f-1 or 1-f-2: 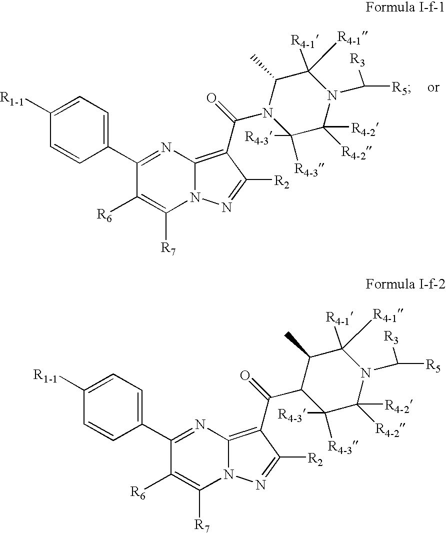
or a pharmaceutically acceptable salt or solvate thereof.

9. The compound according to claim 1 having Formula I-g-1 or 1-g-2: 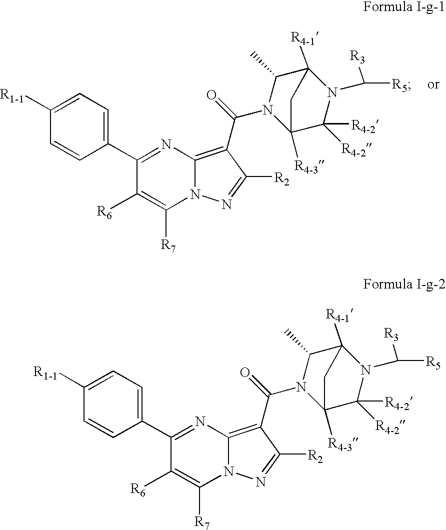
or a pharmaceutically acceptable salt or solvate thereof.

10. The compound according to claim 1 having Formula I-h: 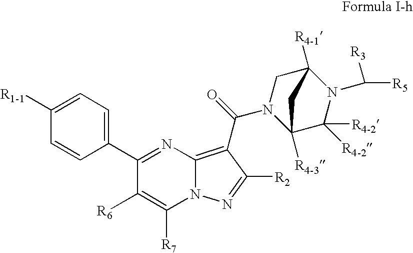
or a pharmaceutically acceptable salt or solvate thereof.

11. The compound according to claim 1 having Formula I-i: 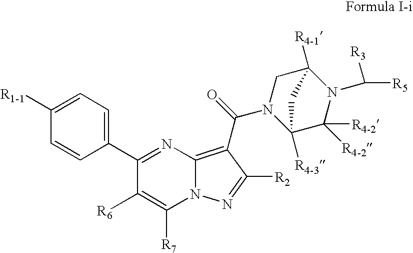
or a pharmaceutically acceptable salt or solvate thereof.

12. The compound according to claim 1 having Formula I-j: 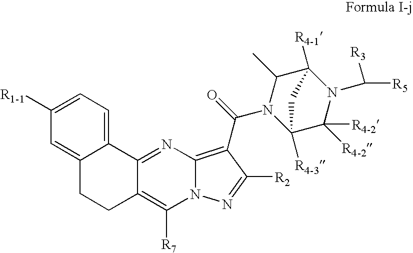
or a pharmaceutically acceptable salt or solvate thereof.

13. The compound according to claim 1 having Formula I-k: 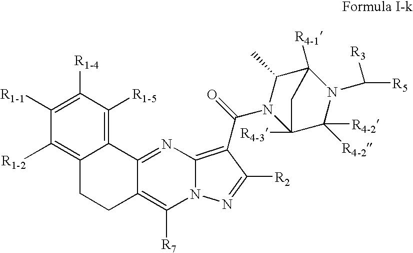
or a pharmaceutically acceptable salt or solvate thereof.

14. The compound or a pharmaceutically acceptable salt or solvate thereof according to claim 1 wherein R₁₋₁ is hydroxyl, alkoxy, alkyl, cycloalkyl, dialkylamino, halo, haloalkyl or nitro.

15. The compound or a pharmaceutically acceptable salt or solvate thereof according to claim 1 wherein R₁₋₁ is methyl, methoxy, ethyl, ethoxy, cyclopropyl, dimethylamino, chloro, fluoro, trifluoromethyl or nitro.

16. (canceled)

17. The compound or a pharmaceutically acceptable salt or solvate thereof according to claim 1 wherein R₁₋₁ is methoxy or ethoxy.

18. (canceled)

19. The compound or a pharmaceutically acceptable salt or solvate thereof according to claim 1 wherein R₁₋₁ is methoxy and R₁₋₂ is hydroxyl, alkoxy, alkyl, or halo.

20. The compound or a pharmaceutically acceptable salt or solvate thereof according to claim 1 wherein R₁₋₁ is alkoxy and R₁₋₂, R₁₋₃, R₁₋₄, and R₁₋₅ are hydrido.

21. The compound or a pharmaceutically acceptable salt or solvate thereof according to claim 1 wherein R₁₋₁ is methoxy and R₁₋₂, R₁₋₃, R₁₋₄, and R₁₋₅ are hydrido.

22. The compound or a pharmaceutically acceptable salt or solvate thereof according to claim 1 wherein R₁₋₁ is alkoxy, R₁₋₂, R₁₋₄, and R₁₋₅ are hydrido, and R₁₋₃ and R₆ together form an ethano bridge.

23. The compound or a pharmaceutically acceptable salt or solvate thereof according to claim 1 wherein R₁₋₁ is methoxy, R₁₋₂, R₁₋₄, and R₁₋₅ are hydrido, and R₁₋₃ and R₆ together form an ethano bridge.

24. The compound or a pharmaceutically acceptable salt or solvate thereof according to claim 5 wherein R₁₋₃ is methoxy or ethoxy.

25. (canceled)

26. The compound or a pharmaceutically acceptable salt or solvate thereof according to claim 1 wherein R₁₋₄ is methoxy or ethoxy, and Y is C.

27. (canceled)

28. The compound or a pharmaceutically acceptable salt or solvate thereof according to claim 1 wherein R₁₋₅ is methoxy or ethoxy.

29. (canceled)

30. The compound or a pharmaceutically acceptable salt or solvate thereof according to claim 1 wherein R₃ is selected from the group consisting of alkyl, cycloalkyl, haloalkyl, hydroxyalkyl, alkoxyalkyl, cyano, aminocarbonyl, alkylaminocarbonyl, alkylcarbonylaminoalkyl, alkylsulfonylaminoalkyl aminoalkyl, alkylaminoalkyl, dialkylaminoalkyl, and N-alkyl-N-alkenylaminoalkyl.

31. The compound or a pharmaceutically acceptable salt or solvate thereof according to claim 1 wherein R₃ is alkyl, haloalkyl, hydroxyalkyl, or alkoxyalkyl.

32. The compound or a pharmaceutically acceptable salt or solvate thereof according to claim 1 wherein R₃ is methyl or ethyl.

33. (canceled)

34. (canceled)

35. The compound or a pharmaceutically acceptable salt or solvate thereof according to claim 1 wherein R₃ is cyclopropyl, cyano, aminocarbonyl, alkylaminocarbonyl, alkylcarbonylaminoalkyl or alkylsulfonylaminoalkyl.

36. (canceled)

37. (canceled)

38. The compound or a pharmaceutically acceptable salt or solvate thereof according to claim 1 wherein R₃ is fluoromethyl, difluoromethyl or trifluoromethyl.

39. (canceled)

40. The compound or a pharmaceutically acceptable salt or solvate thereof according to claim 1 wherein R₃ is hydroxymethyl, 1-hydroxyethyl, 2-hydroxyethyl or 1-hydroxy-1-methylethyl.

41. (canceled)

42. The compound or a pharmaceutically acceptable salt or solvate thereof according to claim 1 wherein R₃ is methoxymethyl or ethoxymethyl.

43. The compound or a pharmaceutically acceptable salt or solvate thereof according to claim 1 wherein R₃ is aminoalkyl, monoalkylaminoalkyl, dialkylaminoalkyl or N-alkyl-N-alkenylaminoalkyl.

44. The compound or a pharmaceutically acceptable salt or solvate thereof according to claim 1 wherein R₃ is N-methylaminomethyl, N,N-dimethylaminomethyl or N-allyl-N-methylaminomethyl.

45. (canceled)

46. The compound or a pharmaceutically acceptable salt or solvate thereof according to claim 1 wherein R₄₋₄′ is methyl, (R) methyl or (S) methyl.

47. (canceled)

48. The compound or a pharmaceutically acceptable salt or solvate thereof according to claim 1 wherein R₅ is selected from the group consisting of phenyl, naphthyl, pyridinyl, pyrazinyl, pyrimidinyl, furanyl, pyrrolyl, thienyl, pyrazolyl, imidazolyl, isoxazolyl, oxazolyl, thiazolyl, triazolyl, oxadiazolyl, thiadiazolyl, tetrazolyl, benzofuranyl, benzothienyl, indolyl, indazolyl, benzoxazolyl, benzimidazolyl, benzothiazolyl, benzoxazolyl, and benzodioxolanyl, any of which is optionally substituted with one or more groups independently selected from the group consisting of halo, cyano, nitro, alkyl, cycloalkyl, haloalkyl, hydroxyalkyl, alkoxyalkyl, aminoalkyl, monoalkylaminoalkyl, dialkylaminoalkyl, alkylcarbonylaminoalkyl, alkylsulfonylaminoalkyl, arylalkyl, formyl, alkanoyl, carboxyl, alkoxycarbonyl, alkoxyalkanoyl, aminocarbonyl, alkylaminocarbonyl, dialkylaminocarbonyl, amino, alkylamino, dialkylamino, alkanoylamino, alkylsulfonylamino, alkoxycarbonylamino, aminocarbonylamino, alkylaminocarbonylamino, dialkylaminocarbonylamino, azido, hydroxyl, alkoxy, haloalkoxy, alkylcarbonyloxy, aminocarbonyloxy, alkylaminocarbonyloxy, dialkylaminocarbonyloxy, amido, alkylamido, alkylamidoalkyl, alkylthio, alkylsulfinyl, alkylsulfonyl, aminosulfonyl, alkylaminosulfonyl, dialkylaminosulfonyl, pyrrolidinyl, piperidinyl, morpholinyl, piperazinyl, and 4-alkylpiperazinyl.

49. The compound or a pharmaceutically acceptable salt or solvate thereof according to claim 48 wherein R₅ is selected from the group consisting of phenyl, naphthyl, pyrid-2-yl, pyrid-3-yl, pyrid-4-yl, pyrazinyl, pyrimidin-5-yl, furan-2-yl, furan-3-yl, pyrrolyl, thien-2-yl, thien-3-yl, pyrazol-3-yl, imidazolyl, isoxazolyl, oxazol-5-yl, thiazol-2-yl, triazolyl, oxadiazolyl, thiadiazolyl, tetrazolyl, benzofuranyl, benzothienyl, indolyl, indazolyl, benzoxazolyl, benzimidazolyl, benzothiazolyl, benzoxazolyl, and benzodioxolanyl, any of which is optionally substituted with one or more groups.

50. The compound or a pharmaceutically acceptable salt or solvate thereof according to claim 1 wherein R₅ is a 5 membered ring comprising at least one unsaturation and 1 heteroatom selected from the group consisting of N, O, and S.

51. The compound or a pharmaceutically acceptable salt or solvate thereof according to claim 1 wherein R₅ is thienyl, furanyl, pyrrolyl, α-naphthyl, quinolin-2-yl, or 1,3-benzodioxolanyl, any of which is optionally substituted.

52. The compound or a pharmaceutically acceptable salt or solvate thereof according to claim 1 wherein R₅ is Formula R:
Formula R= 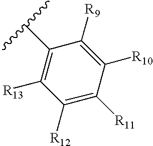
where
R₉, R₁₀, R₁₁, R₁₂ and R₁₃ are independently selected from the group consisting of hydrido, cyano, halo, alkoxy, alkyl, methylthio, azido, hydroxyl, amino, acetamido, methylsulfonylamino, trifluoromethyl, trifluoromethoxy, 1-pyrrolidinyl, cyclopropylcarbonylamino, acetyl, and methylsulfonyl.

53. The compound or a pharmaceutically acceptable salt or solvate thereof according to claim 1 wherein R₅ is Formula R where two of R₉, R₁₀, R₁₁, R₁₂ and R₁₃ are halo.

54. The compound or a pharmaceutically acceptable salt or solvate thereof according to claim 1 wherein R₅ is Formula R where two of R₉, R₁₀, R₁₁, R₁₂ and R₁₃ are fluoro.

55. (canceled)

56. (canceled)

57. (canceled)

58. (canceled)

59. The compound or a pharmaceutically acceptable salt or solvate thereof according to claim 1 wherein R₅ is Formula R where three of R₉, R₁₀, R₁₁, R₁₂ and R₁₃ are halo.

60. The compound or a pharmaceutically acceptable salt or solvate thereof according to claim 1 wherein R₅ is Formula R where three of R₉, R₁₀, R₁₁, R₁₂ and R₁₃ are fluoro.

61. (canceled)

62. The compound or a pharmaceutically acceptable salt or solvate thereof according to claim 1 wherein R₅ is 2,3-dimethoxyphenyl, 2-fluoro-3-methoxyphenyl, 5-fluoro-3-methoxyphenyl, 2,6-difluoro-3-methoxyphenyl; or 3,5-difluoro-2-methoxyphenyl.

63. (canceled)

64. (canceled)

65. The compound or a pharmaceutically acceptable salt or solvate thereof according to claim 1 wherein R₆ is hydrido, alkyl, halo or alkoxy.

66. The compound or a pharmaceutically acceptable salt or solvate thereof according to claim 1 wherein R₆ is hydrido, methyl, fluoro or methoxy.

67. The compound or a pharmaceutically acceptable salt or solvate thereof according to claim 1 wherein R₇ is alkoxycarbonyl, haloalkyl or dialkylamino.

68. (canceled)

69. The compound or a pharmaceutically acceptable salt or solvate thereof according to claim 1 wherein R₇ is trifluoromethyl, difluoromethyl or dimethylamino.

70. (canceled)

71. (canceled)

72. (canceled)

73. The compound or a pharmaceutically acceptable salt or solvate thereof according to claim 1 wherein R₂ is hydrido or hydroxyl.

74. (canceled)

75. A hydrochloride salt or methanesulfonate salt of a compound recited in claim 1.

76. A mono-hydrochloride salt, di-hydrochloride salt, mono-methanesulfonate sale or bis-methanesulfonate salt of a compound recited in claim 1.

77. (canceled)

78. (canceled)

79. (canceled)

80. (canceled)

81. A compound as defined in claim 1 for use as a medicament.

82. The use of a compound as defined in claim 1, for the manufacture of a medicament to treat a disease for which an HIV inhibitor is desired.

83. A method of preventing, treating or delaying the onset of HIV in a subject in need thereof which comprises administering to the subject a therapeutically effective amount of a compound as defined in claim 1.

84. (canceled)

85. A pharmaceutical composition which comprises the product prepared by combining an effective amount of (a) a compound according to claim 1, and (b) a pharmaceutically acceptable carrier.
